(19) United States
(12) Patent Application Publication
(10) Pub. No.: US 2004/0048253 A1
(43) Pub. Date: Mar. 11, 2004

(54) Molecules for diagnostics and therapeutics

(76) Inventors: Scott R. Panzer, Sunnyvale, CA (US); Peter A. Spiro, Palo Alto, CA (US); Steven C. Banville, Palo Alto, CA (US); Purvi Shah, San Jose, CA (US); Michael S. Chalup, Sunnyvale, CA (US); Simon C Chang, Mountain View, CA (US); Alice J. Chen, San Jose, CA (US); Steven A. D'Sa, East Palo, CA (US); Stefan Amshey, San Francisco, CA (US); Christopher E. Dahl, Fremont, CA (US); Tam C. Dam, San Jose, CA (US); Susan E. Daniels, Palo Alto, CA (US); Gerard E. Dufour, Castro Valley, CA (US); Vincent Flores, Union City, CA (US); Willy T. Fong, San Francisco, CA (US); Lila B. Greenawalt, San Jose, CA (US); Jennifer L. Jackson, Mountain View, CA (US); Anissa L. Jones, San Jose, CA (US); Tommy F. Liu, Daly City, CA (US); Ann M. Roseberry Lincoln, Redwood City, CA (US); Bruce H. Rosen, Menlo Park, CA (US); Frank D. Russo, Rossette Court Sunnyvale, CA (US); Theresa K. Stockdreher, Sunnyvale, CA (US); Abel Daffo, San Jose, CA (US); Rachel J. Wright, Mountain View, CA (US); Pierre E. Yap, Lafayette, CA (US); Jimmy Y. Yu, Fremont, CA (US); Diana L. Bradley, Soquel, CA (US); Shawn R. Bratcher, Mountain View, CA (US); Wensheng Chen, Mountain View, CA (US); Howard J. Cohen, Palo Alto, CA (US); David M. Hodgson, Ann Arbor, MI (US); Stephen E. Lincoln, Redwood City, CA (US); Stuart E. Jackson, Mountain View, CA (US)

Correspondence Address: Incyte Genomics Inc, Legal Department, 3160 Porter Drive, Palo Alto, CA, 94304, US

(21) Appl. No.: 10/220,120

(22) PCT Filed: Feb. 21, 2001
(86) PCT No.: PCT/US01/06059

(51) Int. Cl. C12Q 1/68; C07H 21/04; C07K 14/47; A61K 38/17

(52) U.S. Cl. 435/6; 435/69.1; 435/320.1; 435/325; 530/350; 536/23.5

(57) ABSTRACT

The present invention provides purified human polynucleotides for diagnostics and therapeutics (dithp). Also encompassed are the polypeptides (DITHP) encoded by dithp. The invention also provides for the use of dithp, or complements, oligonucleotides, or fragments thereof in diagnostic assays. The invention further provides for vectors and host cells containing dithp for the expression of DITHP. The invention additionally provides for the use of isolated and purified DITHP to induce antibodies and to screen libraries of compounds and the use of anti-DITHP antibodies in diagnostic assays. Also provided are microarrays containing dithp and methods of use.

### TECHNICAL FIELD

The present invention relates to human molecules and to the use of these sequences ill the diagnosis, study, prevention, and treatment of diseases associated with, as well as effects of exogenous compounds on, the expression of human molecules.

### BACKGROUND OF THE INVENTION

The human genome is comprised of thousands of genes, many encoding gene products that function in the maintenance and growth of the various cells and tissues in the body. Aberrant expression or mutations in these genes and their products is the cause of, or is associated with, a variety of human diseases such as cancer and other cell proliferative disorders, autoimmune/inflammatory disorders, infections, developmental disorders, endocine disorders, metabolic disorders, neurological disorders, gastrointestinal disorders, transport disorders, and connective tissue disorders. The identification of these genes and their products is the basis of an ever-expanding effort to find markers for early detection of diseases, and targets for their prevention and treatment. Therefore, these genes and their products are useful as diagnostics and therapeutics. These genes may encode, for example, enzyme molecules, molecules associated with growth and development, biochemical pathway molecules, extracellular information transmission molecules, receptor molecules, intracellular signaling molecules, membrane transport molecules, protein modification and maintenance molecules, nucleic acid synthesis and modification molecules, adhesion molecules, antigen recognition molecules, secreted and extracellular matrix molecules, cytoskeletal molecules, ribosomal molecules, electron transfer associated molecules, transcription factor molecules, chromatin molecules, cell membrane molecules, and organelle associated molecules.

For example, cancer represents a type of cell proliferative disorder that affects nearly every tissue in the body. A wide variety of molecules, either aberrantly expressed or mutated, can be the cause of, or involved with, various cancers because tissue growth involves complex and ordered patterns of cell proliferation, cell differentiation, and apoptosis. Cell proliferation must be regulated to maintain both the number of cells and their spatial organization. This regulation depends upon the appropriate expression of proteins which control cell cycle progression in response to extracellular signals such as growth factors and other mitogens, and intracellular cues such as DNA damage or nutrient starvation. Molecules which directly or indirectly modulate cell cycle progression fall into several categories, including growth factors and their receptors, second messenger and signal transduction proteins, oncogene products, tumor-suppressor proteins, and mitosis-promoting factors. Aberrant expression or mutations in any of these gene products can result in cell proliferative disorders such as cancer. Oncogenes are genes generally derived from normal genes that, through abnormal expression or mutation, can effect the transformation of a normal cell to a malignant one (oncogenesis). Oncoproteins, encoded by oncogenes, can affect cell proliferation in a variety of ways and include growth factors, growth factor receptors, intracellular signal transducers, nuclear transcription factors, and cell-cycle control proteins. In contrast, tumor-suppressor genes are involved in inhibiting cell proliferation. Mutations which cause reduced function or loss of function in tumor-suppressor genes result in aberrant cell proliferation and cancer. Although many different genes and their products have been found to be associated with cell proliferative disorders such as cancer, many more may exist that are yet to be discovered.

DNA-based arrays can provide a simple way to explore the expression of a single polymorphic gene or a large number of genes. When the expression of a single gene is explored, DNA-based arrays are employed to detect the expression of specific gene variants. For example, a p53 tumor suppressor gene array is used to determine whether individuals are carrying mutations that predispose them to cancer. A cytocbrome p450 gene array is useful to determine whether individuals have one of a number of specific mutations that could result in increased drug metabolism, drug resistance or drug toxicity.

DNA-based array technology is especially relevant for the rapid screening of expression of a large number of genes. There is a growing awareness that gene expression is affected in a global fashion. A genetic predisposition, disease or therapeutic treatment may affect, directly or indirectly, the expression of a large number of genes. In some cases the interactions may be expected, such as when the genes are part of the same signaling pathway. In other cases, such as when the genes participate in separate signaling pathways, the interactions may be totally unexpected. Therefore, DNA based arrays can be used to investigate how genetic predisposition, disease, or therapeutic treatment affects the expression of a large number of genes.

Enzyme Molecules

The cellular processes of biogenesis and biodegradation involve a number of key enzyme classes including oxidoreductases, transferases, hydrolases, lyases, isomerases, and ligases. These enzyme classes are each comprised of numerous substrate-specific enzymes having precise and well regulated functions. These enzymes function by facilitating metabolic processes such as glycolysis, the tricarboxylic cycle, and fatty acid metabolism; synthesis or degradation of amino acids, steroids, phospholipids, alcohols, etc.; regulation of cell signalling, proliferation, inflamation, apoptosis, etc., and through catalyzing critical steps in DNA replication and repair, and the process of translation.

Oxidoreductases

Many pathways of biogenesis and biodegradation require oxidoreductase (dehydrogenase or reductase) activity, coupled to the reduction or oxidation of a donor or acceptor cofactor. Potential cofactors include cytochromes, oxygen, disulfide, iron-sulfur proteins, flavin adenine dinucleotide (FAD), and the nicotinamide adenine dinucleotides NAD and NADP (Newsholme, E. A and A. R. Leech (1983) Biochemistry for the Medical Sciences, John Wiley and Sons, Chichester, U.K, pp. 779-793). Reductase activity catalyzes the transfer of electrons between substrate(s) and cofactor(s) with concurrent oxidation of the cofactor. The reverse dehydrogenase reaction catalyzes the reduction of a cofactor and consequent oxidation of the substrate. Oxidoreductase enzymes are a broad superfamily of proteins that catalyze numerous reactions in all cells of organisms ranging from bacteria to plants to humans. These reactions include metabolism of sugar, certain detoxification reactions in the liver, and the synthesis or degradation of fatty acids, amino acids, glucocorticoids, estrogens, androgens, and prostaglandins. Different family members are named according to the direction in which their reactions are typically catalyzed; thus they may be referred to as oxidoreductases, oxidases, reductases, or dehydrogenases. In addition, family members often have distinct cellular localizations, including the cytosol, the plasma membrane, mitochondrial inner or outer membrane, and peroxisomes.

Short-chain alcohol dehydrogenases (SCADs) are a family of dehydrogenases that only share 15% to 30% sequence identity, with similarity predominantly in the coenzyme binding domain and the substrate binding domain. In addition to the well-known role in detoxification of ethanol, SCADs are also involved in synthesis and degradation of fatty acids, steroids, and some prostaglandins, and are therefore implicated in a variety of disorders such as lipid storage disease, myopathy, SCAD deficiency, and certain genetic disorders. For example, retinol dehydrogenase is a SCAD-family member (Simon, A. et al. (1995) J. Biol. Chem. 270:1107-1112) that converts retinol to retinal, the precursor of retinoic acid. Retinoic acid, a regulator of differentiation and apoptosis, has been shown to down-regulate genes involved in cell proliferation and inflammation (Chai, X. et al. (1995) J. Biol. Chem. 270:3900-3904). In addition, retinol dehydrogenase has been linked to hereditary eye diseases such as autosomal recessive childhood-onset severe retinal dystrophy (Simon, A et al; (1996) Genomics 36:424-430).

Propagation of nerve impulses, modulation of cell proliferation and differentiation, induction of the immune response, and tissue homeostasis involve neurotransmitter metabolism (Weiss, B. (1991) Neurotoxicology 12:379-386; Collins, S. M. et al. (1992) Ann N.Y. Acad. Sci. 664:415424; Brown, J. K. and H. Imam (1991) J. Inherit. Metab. Dis. 14:436-458). Many pathways of neurotransmitter metabolism require oxidoreductase activity, coupled to reduction or oxidation of a cofactor, such as NAD⁺/NADH (Newsholme, E. A. and A. R. Leech (1983) Biochemistry for the Medical Sciences, John Wiley and Sons, Chichester, U.K pp. 779-793). Degradation of catecholamies (epinephrine or norepinephrine) requires alcohol dehydrogenase (in the brain) or aldehyde dehydrogenase (in peripheral tissue). NAD⁺-dependent aldehyde dehydrogenase oxidizes 5-hydroxyindole-3-acetate (the product of 5-hydroxytryptamine (serotonin) metabolism) in the brain, blood platelets, liver and pulmonary endothelium (Newsholme, supra, p. 786). Other neurotransmitter degradation pathways that utilize NAD⁺/NADH-dependent oxidoreductase activity include those of L-DOPA (precursor of dopamine, a neuronal excitatory compound), glycine (an inhibitory neurotransmitter in the brain and spinal cord), histamine (liberated from mast cells during the inflammatory response), and taurine (an inhibitory neurotransmitter of the brain stem, spinal cord and retina) (Newsholme, supra, pp. 790, 792). Epigenetic or genetic defects in neurotransmitter metabolic pathways can result in a spectrum of disease states in different tissues including Parkinson disease and inherited myoclonus (McCance, K. L. and S. E. Huether (1994) Pathophysiology, Mosby-Year Book, Inc., St Louis Mo., pp. 402-404; Gundlach, AL. (1990) FASEB J. 4:2761-2766).

Tetrahydrofolate is a derivatized glutamate molecule that acts as a carrier, providing activated is, one-carbon units to a wide variety of biosynthetic reactions, including synthesis of purines, pyrimidines, and the amino acid methionine. Tetrahydrofolate is generated by the activity of a holoenzyme complex called tetrahydrofolate synthase, which includes three enzyme activities: tetrahydrofolate dehydrogenase, tetrahydrofolate cyclohydrolase, and tetrahydrofolate synthetase. Thus, tetrahydrofolate dehydrogenase plays an important role in generating building blocks for nucleic and amino acids, crucial to proliferating cells.

3-Hydroxyacyl-CoA dehydrogenase (3HACD) is involved in fatty acid metabolism. It catalyzes the reduction of 3-hydroxyacyl-CoA to 3-oxoacyl-CoA, with concomitant oxidation of NAD to NADH, in the mitochondria and peroxisomes of eukaryotic cells. In peroxisomes, 3HACD and enoyl-CoA hydratase form an enzyme complex called bifunctional enzyme, defects in which are associated with peroxisomal bifunctional enzyme deficiency. This interruption in fatty acid metabolism produces accumulation of very-long chain fatty acids, disrupting development of the brain, bone, and adrenal glands. Infants born with this deficiency typically die within 6 months (Watkins, P. et al. (1989) J. Clin. Invest. 83:771-777; Online Mendelian Inheritance in Man (OMIM) #261-515). The neurodegeneration that is characteristic of Alzheimer's disease involves development of extracellular plaques in certain brain regions. A major protein component of these plaques is the peptide amyloid-β (Aβ), which is one of several cleavage products of amyloid precursor protein (APP). 3HACD has been shown to bind the Aβ peptide, and is overexpressed in neurons affected in Alzheimer's disease. In addition, an antibody against 3HACD can block the toxic effects of Aβ in a cell culture model of Alzheimer's disease (Yan, S. et al. (1997) Nature 389:689-695; OMIM, #602057).

Steroids, such as estrogen, testosterone, corticosterone, and others, are generated from a common precursor, cholesterol, and are interconverted into one another. A wide variety of enzymes act upon cholesterol, including a number of dehydrogenases. Steroid dehydrogenases, such as the hydroxysteroid dehydrogenases, are involved in hypertension, fertility, and cancer (Duax, W. L. and D. Ghosh (1997) Steroids 62:95-100). One such dehydrogenase is 3-oxo-5-α-steroid dehydrogenase (OASD), a microsomal membrane protein highly expressed in prostate and other androgen-responsive tissues. OASD catalyzes the conversion of testosterone into dihydrotestosterone, which is the most potent androgen. Dihydrotestosterone is essential for the formation of the male phenotype during embryogenesis, as well as for proper androgen-mediated growth of tissues such as the prostate and male genitalia. A defect in OASD that prevents the conversion of testosterone into dihydrotestosterone leads to a rare form of male pseudohermaphroditis, characterized by defective formation of the external genitalia (Andersson, S. et al. (1991) Nature 354:159-161; Labrie, F. et al. (1992) Endocrinology 131:1571-1573; OMIM #264600). Thus, OASD plays a central role in sexual differentiation and androgen physiology.

17β-hydroxysteroid dehydrogenase (17βHSD6) plays an important role in the regulation of the male reproductive hormone, dihydrotestosterone (DHTT). 17βHSD6 acts to reduce levels of DHTT by oxidizing a precursor of DHTT, 3α-diol, to androsterone which is readily glucuronidated and removed from tissues. 17βHSD6 is active with both androgen and estrogen substrates when expressed in embryonic kidney 293 cells. At least five other isozymes of 17 βHSD have been identified that catalyze oxidation and/or reduction reactions in various tissues with preferences for different steroid substrates (Biswas, M. G. and D. W. Russell (1997) J. Biol. Chem. 272:15959-15966). For example, 17,HSD1 preferentially reduces estradiol and is abundant in the ovary and placenta 17βHSD2 catalyzes oxidation of androgens and is present in the endometrium and placenta. 17βHSD3 is exclusively a reductive enzyme in the testis (Geissler, W. M. et al. (1994) Nat. Genet. 7:34-39). An excess of androgens such as DHTT can contribute to certain disease states such as benign prostatic hyperplasia and prostate cancer.

Oxidoreductases are components of the fatty acid metabolism pathways in mitochondria and peroxisomes. The main beta-oxidation pathway degrades both saturated and unsaturated fatty acids, while the auxiliary pathway performs additional steps required for the degradation of unsaturated fatty acids. The auxiliary beta-oxidation enzyme 2,4-dienoyl-CoA reductase catalyzes the removal of even-numbered double bonds from unsaturated fatty acids prior to their entry into the main beta-oxidation pathway. The enzyme may also remove odd-numbered double bonds from unsaturated fatty acids (Koivuranta, K. T. et al. (1994) Biochem. J. 304:787-792; Smeland, T. E. et al. (1992) Proc.

Natl. Acad. Sci. USA 89:6673-6677). 2,4-dienoyl-CoA reductase is located in both mitochondria and peroxisomes. Inherited deficiencies in mitochondrial and peroxisomal beta-oxidation enzymes are associated with severe diseases, some of which manifest themselves soon after birth and lead to death within a few years. Defects in beta-oxidation are associated with Reye's syndrome, Zellweger syndrome, neonatal adrenoleukodystrophy, infantile Refsum's disease, acyl-CoA oxidase deficiency, and bifunctional protein deficiency (Suzuki, Y. et al. (1994) Am. J. Hum. Genet. 54:36-43; Hoefler, supra; Cotran, R. S. et al. (1994) Robbins Pathologic Basis of Disease, W. B. Saunders Co., Philadelphia Pa., p.866). Peroxisomal beta-oxidation is impaired in cancerous tissue. Although neoplastic human breast epithelial cells have the same number of peroxisomes as do normal cells, fatty acyl-CoA oxidase activity is lower than in control tissue (el Bouhtoury, F. et al. (1992) J. Pathol. 166:27-35). Human colon carcinomas have fewer peroxisomes than normal colon tissue and have lower fatty-acyl-CoA oxidase and bifunctional enzyme (including enoyl-CoA hydratase) activities than normal tissue (Cable, S. et al. (1992) Virchows Arch. B Cell Pathol. Incl. Mol. Pathol. 62:221-226). Another important oxidoreductase is isocitrate dehydrogenase, which catalyzes the conversion of isocitrate to a-ketoglutarate, a substrate of the citric acid cycle. Isocitrate dehydrogenase can be either NAD or NADP dependent, and is found in the cytosol, mitochondria, and peroxisomes. Activity of isocitrate dehydrogenase is regulated developmentally, and by hormones, neurotransmitters, and growth factors.

Hydroxypyruvate reductase (HPR), a peroxisomal 2-hydroxyacid dehydrogenase in the glycolate pathway, catalyzes the conversion of hydroxypyruvate to glycerate with the oxidation of both NADH and NADPH. The reverse dehydrogenase reaction reduces NAD⁺and NADP⁺. HPR recycles nucleotides and bases back into pathways leading to the synthesis of ATP and GTP. ATP and GTP are used to produce DNA and RNA and to control various aspects of signal transduction and energy metabolism. Inhibitors of purine nucleotide biosynthesis have long been employed as antiproliferative agents to treat cancer and viral diseases. HPR also regulates biochemical synthesis of serine and cellular serine levels available for protein synthesis.

The mitochondrial electron transport (or respiratory) chain is a series of oxidoreductase-type enzyme complexes in the mitochondrial membrane that is responsible for the transport of electrons from NADH through a series of redox centers within these complexes to oxygen, and the coupling of this oxidation to the synthesis of ATP (oxidative phosphorylation). ATP then provides the primary source of energy for driving a cell's many energy-requiring reactions. The key complexes in the respiratory chain are NADH:ubiquinone oxidoreductase (complex I), succinate:ubiquinone oxidoreductase (complex II), cytochrome c₁-b oxidoreductase (complex III), cytochrome c oxidase (complex IV), and ATP synthase (complex V) (Alberts, B. et al. (1994) Molecular Biology of the Cell, Garland Publishing, Inc., New York N.Y., pp. 677-678). All of these complexes are located on the inner matrix side of the mitochondrial membrane except complex II, which is on the cytosolic side. Complex II transports electrons generated in the citric acid cycle to the respiratory chain. The electrons generated by oxidation of succinate to fumarate in the citric acid cycle are transferred through electron carriers in complex II to membrane bound ubiquinone (Q). Transcriptional regulation of these nuclear-encoded genes appears to be the predominant means for controlling the biogenesis of respiratory enzymes. Defects and altered expression of enzymes in the respiratory chain are associated with a variety of disease conditions.

Other dehydrogenase activities using NAD as a cofactor are also important in mitochondrial function. 3-hydroxyisobutyrate dehydrogenase (3HBD), important in valine catabolism, catalyzes the NAD-dependent oxidation of 3-hydroxyisobutyrate to methylmalonate semialdehyde within mitochondria. Elevated levels of 3-hydroxyisobutyrate have been reported in a number of disease states, including ketoacidosis, methylmalonic acidemia, and other disorders associated with deficiencies in methylmalonate semialdehyde dehydrogenase (Rougraff, P. M. et al. (1989) J. Biol. Chem. 264:5899-5903).

Another mitochondrial dehydrogenase important in amino acid metabolism is the enzyme isovaleryl-CoA-dehydrogenase (IVD). IVD is involved in leucine metabolism and catalyzes the oxidation of isovaleryl-CoA to 3-methylcrotonyl-CoA. Human IVD is a tetrameric flavoprotein that is encoded in the nucleus and synthesized in the cytosol as a 45 kDa precursor with a mitochondrial import signal sequence. A genetic deficiency, caused by a mutation in the gene encoding IVD, results in the condition known as isovaleric acidemia. This mutation results in inefficient mitochondrial import and processing of the IVD precursor (Vockley, J. et al. (1992) J. Biol. Chem. 267:2494-2501).

Transferases

Transferases are enzymes that catalyze the transfer of molecular groups. The reaction may involve an oxidation, reduction, or cleavage of covalent bonds, and is often specific to a substrate or to particular sites on a type of substrate. Transferases participate in reactions essential to such functions as synthesis and degradation of cell components, regulation of cell functions including cell signaling, cell proliferation, inflamation, apoptosis, secretion and excretion. Transferases are involved in key steps in disease processes involving these functions. Transferases are frequently classified according to the type of group transferred. For example, methyl transferases transfer one-carbon methyl groups, amino transferases transfer nitrogenous amino groups, and similarly denominated enzymes transfer aldehyde or ketone, acyl, glycosyl, alkyl or aryl, isoprenyl, saccharyl, phosphorous-containing, sulfur-containing, or selenium-containing groups, as well as small enzymatic groups such as Coenzyme A

Acyl transferases include peroxisomal carnitine octanoyl transferase, which is involved in the fatty acid beta-oxidation pathway, and mitochondrial carnitine palmitoyl transferases, involved in fatty acid metabolism and transport. Choline O-acetyl transferase catalyzes the biosynthesis of the neurotransmitter acetylcholine.

Amino transferases play key roles in protein synthesis and degradation, and they contribute to other processes as well. For example, the amino transferase 5-aminolevulinic acid synthase catalyzes the addition of succinyl-CoA to glycine, the first step in heme biosynthesis. Other amino transferases participate in pathways important for neurological function and metabolism. For example, glutamine-phenylpyruvate amino transferase, also known as glutamine transaminase K (GTK), catalyzes several reactions with a pyridoxal phosphate cofactor. GTK catalyzes the reversible conversion of L-glutamine and phenylpyruvate to 2-oxoglutaramate and L-phenylalanine. Other amino acid substrates for GTK include L-methionine, L-histidine, and L-tyrosine. GTK also catalyzes the conversion of kynurenine to kynurenic acid, a tryptophan metabolite that is an antagonist of the N-methyl-D-aspartate (NMDA) receptor in the brain and may exert a neuromodulatory function. Alteration of the kynurenine metabolic pathway may be associated with several neurological disorders. GTK also plays a role in the metabolism of halogenated xenobiotics conjugated to glutathione, leading to nephrotoxicity in rats and neurotoxicity in humans. GTK is expressed in kidney, liver, and brain. Both human and rat GTKs contain a putative pyridoxal phosphate binding site (ExPASy ENZYME: EC 2.6.1.64; Perry, S. J. et al. (1993) Mol. Pharmacol. 43:660-665; Perry, S. et al. (1995) FEBS Lett 360:277-280; and Alberati-Giani, D. et al. (1995) J. Neurochem. 64:1448-1455). A second amino transferase associated with this pathway is kynurenine/α-aminoadipate amino transferase (AadAT. AadAT catalyzes the reversible conversion of α-aminoadipate and α-ketoglutarate to α-ketoadipate and L-glutamate during lysine metabolism. AadAT also catalyzes the transamination of kynurenine to kynurenic acid. A cytosolic AadAT is expressed in rat kidney, liver, and brain (Nakatani, Y. et al. (1970) Biochim Biophys. Acta 198:219-228; Buchli, R. et al. (1995) J. Biol. Chem. 270:29330-29335).

Glycosyl transferases include the mammalian UDP-glucouronosyl transferases, a family of membrane-bound microsomal enzymes catalyzing the transfer of glucouronic acid to lipophilic substrates in reactions that play important roles in detoxification and excretion of drugs, carcinogens, and other foreign substances. Another mammalian glycosyl transferase, mammalian UDP-galactose-ceramide galactosyl transferase, catalyzes the transfer of galactose to ceramide in the synthesis of galactocerebrosides in myelin membranes of the nervous system. The UDP-glycosyl transferases share a conserved signature domain of about 50 amino acid residues (PROSITE: PDOC00359, http://expasyhcuge.ch/sprotfprosite html).

Methyl transferases are involved in a variety of pharmacologically important processes. Nicotinamide N-methyl transferase catalyzes the N-methylation of nicotinamides and other pyridines, an important step in the cellular handling of drugs and other foreign compounds. Phenylethanolamine N-methyl transferase catalyzes the conversion of noradrenalin to adrenalin 6-O-methylguanine-DNA methyl transferase reverses DNA methylation, an important step in carcinogenesis. Uroporphyrin-III C-methyl transferase, which catalyzes the transfer of two methyl groups from S-adenosyl-L-methionine to uroporphyrinogen III, is the first specific enzyme in the biosynthesis of cobalamin, a dietary enzyme whose uptake is deficient in pernicious anemia. Protein-arginine methyl transferases catalyze the posttranslational methylation of arginine residues in proteins, resulting in the mono- and dimethylation of arginine on the guanidino group. Substrates include histones, myelin basic protein, and heterogeneous nuclear ribonucleoproteins involved in mRNA processing, splicing, and transport. Protein-arginine methyl transferase interacts with proteins upregulated by mitogens, with proteins involved in chronic lymphocytic leukemia, and with interferon, suggesting an important role for methylation in cytokine receptor signaling (Lin, W. -J. et is al. (1996) J. Biol. Chem. 271:15034-15044; Abramovich, C. et al. (1997) EMBO J. 16:260-266; and Scott, H. S. et al. (1998) Genomics 48:330-340).

Phosphotransferases catalyze the transfer of high-energy phosphate groups and are important in energy-requiring and-releasing reactions. The metabolic enzyme creatine kinase catalyzes the reversible phosphate transfer between creatine/creatine phosphate and ATP/ADP. Glycocyamine kinase catalyzes phosphate transfer from ATP to guanidoacetate, and arginine kinase catalyzes phosphate transfer from ATP to arginine. A cysteine-containing active site is conserved in this family (PROSITE: PDOC00103).

Prenyl transferases are heterodimers, consisting of an alpha and a beta subunit, that catalyze the transfer of an isoprenyl group. An example of a prenyl transferase is the mammalian protein farnesyl transferase. The alpha subunit of farnesyl transferase consists of 5 repeats of 34 amino acids each, with each repeat containing an invariant tryptophan (PROSITE: PDOC00703).

Saccharyl transferases are glycating enzymes involved in a variety of metabolic processes. Oligosacchryl transferase-48, for example, is a receptor for advanced glycation endproducts. Accumulation of these endproducts is observed in vascular complications of diabetes, macrovascular disease, renal insufficiency, and Alzheimer's disease (Thornalley, P. J. (1998) Cell Mol. Biol. (Noisy-Le-Grand) 44:1013-1023).

Coenzyme A (CoA) transferase catalyzes the transfer of CoA between two carboxylic acids. Succinyl CoA.3-oxoacid CoA transferase, for example, transfers CoA from succinyl-CoA to a recipient such as acetoacetate. Acetoacetate is essential to the metabolism of ketone bodies, which accumulate in tissues affected by metabolic disorders such as diabetes (PROSITE: PDOC00980).

Hydrolases

Hydrolysis is the breaking of a covalent bond in a substrate by introduction of a molecule of water. The reaction involves a nucleophilic attack by the water molecule's oxygen atom on a target bond in the substrate. The water molecule is split across the target bond, breaking the bond and generating two product molecules. Hydrolases participate in reactions essential to such functions as synthesis and degradation of cell components, and for regulation of cell functions including cell signaling, cell proliferation, inflamation, apoptosis, secretion and excretion. Hydrolases are involved in key steps in disease processes involving these functions. Hydrolytic enzymes, or hydrolases, may be grouped by substrate specificity into classes including phosphatases, peptidases, lysophospholipases, phosphodiesterases, glycosidases, and glyoxalases.

Phosphatases hydrolytically remove phosphate groups from proteins, an energy-providing step that regulates many cellular processes, including intracellular signaling pathways that in turn control cell growth and differentiation, cell-cell contact, the cell cycle, and oncogenesis.

Lysophospholipases (LPLs) regulate intracellular lipids by catalyzing the hydrolysis of ester bonds to remove an acyl group, a key step in lipid degradation. Small LPL isoforms, approximately 15-30 kD, function as hydrolases; larger isoforms function both as hydrolases and transacylases. A particular substrate for LPLs, lysophosphatidylcholine, causes lysis of cell membranes. LPL activity is regulated by signaling molecules important in numerous pathways, including the inflammatory response.

Peptidases, also called proteases, cleave peptide bonds that form the backbone of peptide or protein chains. Proteolytic processing is essential to cell growth, differentiation, remodeling, and homeostasis as well as inflammation and immune response. Since typical protein half-lives range from hours to a few days, peptidases are continually cleaving precursor proteins to their active form, removing signal sequences from targeted proteins, and degrading aged or defective proteins. Peptidases function in bacterial, parasitic, and viral invasion and replication within a host. Examples of peptidases include trypsin and chymotrypsin (components of the complement cascade and the blood-clotting cascade) lysosomal cathepsins, calpains, pepsin, renin, and chymosin (Beynon, R. J. and J. S. Bond (1994) Proteolytic Enzymes: A Practical Approach, Oxford University Press, New York N.Y., pp. 1-5).

The phosphodiesterases catalyze the hydrolysis of one of the two ester bonds in a phosphodiester compound. Phosphodiesterases are therefore crucial to a variety of cellular processes. Phosphodiesterases include DNA and RNA endo- and exo-nucleases, which are essential to cell growth and replication as well as protein synthesis. Another phosphodiesterase is acid sphingomyelinase, which hydrolyzes the membrane phosphoilpid sphingomyelin to ceramide and phosphorylcholine. Phosphorylcholine is used in the synthesis of phosphatidylcholine, which is involved in numerous intracellular signaling pathways. Ceramide is an essential precursor for the generation of gangliosides, membrane lipids found in high concentration in neural tissue. Defective acid sphingomyelinase phosphodiesterase leads to a build-up of sphingomyelin molecules in lysosomes, resulting in Niemann-Pick disease.

Glycosidases catalyze the cleavage of hemiacetyl bonds of glycosides, which are compounds that contain one or more sugar. Mammalian lactase-phlorizin hydrolase, for example, is an intestinal enzyme that splits lactose. Mammalian beta-galactosidase removes the terminal galactose from gangliosides, glycoproteins, and glycosaminoglycans, and deficiency of this enzyme is associated with a gangliosidosis known as Morquio disease type B. Vertebrate lysosomal alpha-glucosidase, which hydrolyzes glycogen, maltose, and isomaltose, and vertebrate intestinal sucrase-isomaltase, which hydrolyzes sucrose, maltose, and isomaltose, are widely distributed members of this family with highly conserved sequences at their active sites.

The glyoxylase system is involved in gluconeogenesis, the production of glucose from storage compounds in the body. It consists of glyoxylase I, which catalyzes the formation of S-D-lactoylglutathione from methyglyoxal, a side product of triose-phosphate energy metabolism, and glyoxylase II, which hydrolyzes S-D-lactoylglutathione to D-lactic acid and reduced glutathione. Glyoxylases are involved in hyperglycemia, non-insulin-dependent diabetes mellitus, the detoxification of bacterial toxins, and in the control of cell proliferation and microtubule assembly.

Lyases

Lyases are a class of enzymes that catalyze the cleavage of C—C, C—O, C—N, C—S, C-(halide), P—O or other bonds without hydrolysis or oxidation to form two molecules, at least one of which contains a double bond (Stryer, L. (1995) Biochemistry W. H. Freeman and Co. New York, N.Y. p.620). Lyases are critical components of cellular biochemistry with roles in metabolic energy production including fatty acid metabolism, as well as other diverse enzymatic processes. Further classification of lyases reflects the type of bond cleaved as well as the nature of the cleaved group.

The group of C—C lyases include carboxyl-lyases (decarboxylases), aldehyde-lyases (aldolases), oxo-acid-lyases and others. The C—O lyase group includes hydro-lyases, lyases acting on polysaccharides and other lyases. The C—N lyase group includes ammonia-lyases, amidine-lyases, amine-lyases (deaminases) and other lyases.

Proper regulation of lyases is critical to normal physiology. For example, mutation induced deficiencies in the uroporphyrinogen decarboxylase can lead to photosensitive cutaneous lesions in the genetically-linked disorder familial porphyria cutanea tarda (Mendez, M. et al. (1998) Am. J. Genet. 63:1363-1375). It has also been shown that adenosine deaminase (ADA) deficiency stems from genetic mutations in the ADA gene, resulting in the disorder severe combined immunodeficiency disease (SCID) (Hershfield, M. S. (1998) Semin. Hematol. 35:291-298).

Isomerases

Isomerases are a class of enzymes that catalyze geometric or structural changes within a molecule to form a single product. This class includes racemases and epimerases, cis-trans-isomerases, intramolecular oxidoreductases, intramolecular transferases (mutases) and intramolecular lyases. Isomerases are critical components of cellular biochemistry with roles in metabolic energy production including glycolysis, as well as other diverse enzymatic processes (Stryer, L. (1995) Biochemistry, W. H. Freeman and Co., New York N.Y., pp.483-507).

Racemases are a subset of isomerases that catalyze inversion of a molecules configuration around the asymmetric carbon atom in a substrate having a single center of asymmetry, thereby interconverting two racemers. Epimerases are another subset of isomerases that catalyze inversion of configuration around an asymmetric carbon atom in a substrate with more than one center of symmetry, thereby interconverting two epimers. Racemases and epimerases can act on amino acids and derivatives, hydroxy acids and derivatives, as well as carbohydrates and derivatives. The interconversion of UDP-galactose and UDP-glucose is catalyzed by UDP-galactose4′-epimerase. Proper regulation and function of this epimerase is essential to the synthesis of glycoproteins and glycolipids. Elevated blood galactose levels have been correlated with UDP-galactose4′-epimerase deficiency in screening programs of infants (Gitzelmann, R. (1972) Helv. Paediat. Acta 27:125-130).

Oxidoreductases can be isomerases as well. Oxidoreductases catalyze the reversible transfer of electrons from a substrate that becomes oxidized to a substrate that becomes reduced. This class of enzymes includes dehydrogenases, hydroxylases, oxidases, oxygenases, peroxidases, and reductases. Proper maintenance of oxidoreductase levels is physiologically important. For example, genetically-linked deficiencies in lipoamide dehydrogenase can result in lactic acidosis (Robinson, B. H. et al. (1977) Pediat. Res. 11:1198-1202).

Another subgroup of isomerases are the transferases (or mutases). Transferases transfer a chemical group from one compound (the donor) to another compound (the acceptor). The types of groups transferred by these enzymes include acyl groups, amino groups, phosphate groups (phosphotransferases or phosphomutases), and others. The transferase carnitine palmitoyltransferase is an important component of fatty acid metabolism. Genetically-linked deficiencies in this transferase can lead to myopathy (Scriver, C. R. et al. (1995) The Metabolic and Molecular Basis of Inherited Disease, McGraw-Hill, New York N.Y., pp.1501-1533).

Yet another subgroup of isomerases are the topoisomersases. Topoisomerases are enzymes that affect the topological state of DNA. For example, defects in topoisomerases or their regulation can affect normal physiology. Reduced levels of topoisomerase II have been correlated with some of the DNA processing defects associated with the disorder ataxia-telangiectasia (Singh, S. P. et al. (1988) Nucleic Acids Res. 16:3919-3929).

Ligases

Ligases catalyze the formation of a bond between two substrate molecules. The process involves the hydrolysis of a pyrophosphate bond in ATP or a similar energy donor. Ligases are classified based on the nature of the type of bond they form, which can include carbon-oxygen, carbon-sulfur, carbon-nitrogen, carbon-carbon and phosphoric ester bonds.

Ligases forming carbon-oxygen bonds include the aminoacyl-transfer RNA (tRNA) synthetases which are important RNA-associated enzymes with roles in translation. Protein biosynthesis depends on each amino acid forming a linkage with the appropriate tRNA. The aminoacyl-tRNA synthetases are responsible for the activation and correct attachment of an ammo acid with its cognate tRNA. The 20 aminoacyl-tRNA synthetase enzymes can be divided into two structural classes, and each class is characterized by a distinctive topology of the catalytic domain.

Class I enzymes contain a catalytic domain based on the nucleotide-binding Rossman fold. Class II enzymes contain a central catalytic domain, which consists of a seven-stranded antiparallel β-sheet motif, as well as N- and C-terminal regulatory domains. Class II enzymes are separated into two groups based on the heterodimeric or homodimeric structure of the enzyme; the latter group is further subdivided by the structure of the N- and C-terminal regulatory domains (Hartlein, M. and S. Cusack (1995) J. Mol. Evol. 40:519-530). Autoantibodies against aminoacyl-tRNAs are generated by patients with dermatomyositis and polymyositis, and correlate strongly with complicating interstitial lung disease (ILD). These antibodies appear to be generated in response to viral infection, and coxsackie virus has been used to induce experimental viral myositis in animals.

Ligases forming carbon-sulfur bonds (Acid-thiol ligases) mediate a large number of cellular biosynthetic intermediary metabolism processes involve intermolecular transfer of carbon atom-containing substrates (carbon substrates). Examples of such reactions include the tricarboxylic acid cycle, synthesis of fatty acids and long-chain phospholipids, synthesis of alcohols and aldehydes, synthesis of intermediary metabolites, and reactions involved in the amino acid degradation pathways. Some of these reactions require input of energy, usually in the form of conversion of ATP to either ADP or AMP and pyrophosphate.

In many cases, a carbon substrate is derived from a small molecule containing at least two carbon atoms. The carbon substrate is often covalently bound to a larger molecule which acts as a carbon substrate carrier molecule within the cell. In the biosynthetic mechanisms described above, the carrier molecule is coenzyme A. Coenzyme A (CoA) is structurally related to derivatives of the nucleotide ADP and consists of 4′-phosphopantetheine linked via a phosphodiester bond to the alpha phosphate group of adenosine 3′,5′-bisphosphate. The terminal thiol group of 4′-phosphopantetheine acts as the site for carbon substrate bond formation. The predominant carbon substrates which utilize CoA as a carrier molecule during biosynthesis and intermediary metabolism in the cell are acetyl, succinyl, and propionyl moieties, collectively referred to as acyl groups. Other carbon substrates include enoyl lipid, which acts as a fatty acid oxidation intermediate, and carnitine, which acts as an acetyl-CoA flux regulator/mitochondrial acyl group transfer protein. Acyl-CoA and acetyl-CoA are synthesized in the cell by acyl-CoA synthetase and acetyl-CoA synthetase, respectively.

Activation of fatty acids is mediated by at least three forms of acyl-CoA synthetase activity: i) acetyl-CoA synthetase, which activates acetate and several other low molecular weight carboxylic acids and is found in muscle mitochondria and the cytosol of other tissues; ii) medium-chain acyl-CoA synthetase, which activates fatty acids containing between four and eleven carbon atoms (predominantly from dietary sources), and is present only in liver mitochondria; and iii) acyl CoA synthetase, which is specific for long chain fatty acids with between six and twenty carbon atoms, and is found in microsomes and the mitochondria. Proteins associated with acyl-CoA synthetase activity have been identified from many sources including bacteria, yeast, plants, mouse, and man. The activity of acyl-CoA synthetase may be modulated by phosphorylation of the enzyme by cAMP-dependent protein kinase.

Ligases forming carbon-nitrogen bonds include amide synthases such as glutamine synthetase (glutamate-ammonia ligase) that catalyzes the amination of glutamic acid to glutamine by ammonia using the energy of ATP hydrolysis. Glutamine is the primary source for the amino group in various amide transfer reactions involved in de novo pyrimidine nucleotide synthesis and in purine and pyrimidine ribonucleotide interconversions. Overexpression of glutamine synthetase has been observed in primary liver cancer (Christa, L. et al. (1994) Gastroent. 106:1312-1320).

Acid-amino-acid ligases (peptide synthases) are represented by the ubiquitin proteases which are associated with the ubiquitin conjugation system (UCS), a major pathway for the degradation of cellular proteins in eukaryotic cells and some bacteria. The UCS mediates the elimination of abnormal proteins and regulates the half-lives of important regulatory proteins that control cellular processes such as gene transcription and cell cycle progression. In the UCS pathway, proteins targeted for degradation are conjugated to a ubiquitin (Ub), a small heat stable protein. Ub is first activated by a ubiquitin-activating enzyme (E1), and then transferred to one of several Ub-conjugating enzymes (E2). E2 then links the Ub molecule through its C-terminal glycine to an internal lysine (acceptor lysine) of a target protein. The ubiquitinated protein is then recognized and degraded by proteasome, a large, multisubunit proteolytic enzyme complex, and ubiquitin is released for reutilization by ubiquitin protease. The UCS is implicated in the degradation of mitotic cyclic kinases, oncoproteins, tumor suppressor genes such as p53, viral proteins, cell surface receptors associated with signal transduction, transcriptional regulators, and mutated or damaged proteins (Ciechanover, A (1994) Cell 79:13-21). A murine proto-oncogene, Unp, encodes a nuclear ubiquitin protease whose overexpression leads to oncogenic transformation of NIH3T3 cells, and the human homolog of this gene is consistently elevated in small cell tumors and adenocarcinomas of the lung (Gray, D. A. (1995) Oncogene 10:2179-2183).

Cyclo-ligases and other carbon-nitrogen ligases comprise various enzymes and enzyme complexes that participate in the de novo pathways to purine and pyrimidine biosynthesis. Because these pathways are critical to the synthesis of nucleotides for replication of both RNA and DNA, many of these enzymes have been the targets of clinical agents for the treatment of cell proliferative disorders such as cancer and infectious diseases.

Purine biosynthesis occurs de novo from the amino acids glycine and glutamine, and other small molecules. Three of the key reactions in this process are catalyzed by a trifunctional enzyme composed of glycinamide-ribonucleotide synthetase (GARS), aminoimidazole ribonucleotide synthetase (AIRS), and glycinamide ribonucleotide transformylase (GART). Together these three enzymes combine ribosylamine phosphate with glycine to yield phosphoribosyl aminoimidazole, a precursor to both adenylate and guanylate nucleotides. This trifunctional protein has been implicated in the pathology of Downs syndrome (Aimi, J. et al. (1990) Nucleic Acid Res. 18:6665-6672). Adenylosuccinate synthetase catalyzes a later step in purine biosynthesis that converts inosinic acid to adenylosuccinate, a key step on the path to ATP synthesis. This enzyme is also similar to another carbon-nitrogen ligase, argininosuccinate synthetase, that catalyzes a similar reaction in the urea cycle (Powell, S. M. et al. (1992) FEBS Lett. 303:4-10).

Like the de novo biosynthesis of purines, de novo synthesis of the pyrimidine nucleotides uridylate and cytidylate also arises from a common precursor, in this instance the nucleotide orotidylate derived from orotate and phosphoribosyl pyrophosphate (PPRP). Again a trifunctional enzyme comprising three carbon-nitrogen ligases plays a key role in the process. In this case the enzymes aspartate transcarbamylase (ATCase), carbamyl phosphate synthetase II, and dihydroorotase (DHOase) are encoded by a single gene called CAD. Together these three enzymes combine the initial reactants in pyrimidine biosynthesis, glutamine, CO₂, and ATP to form dihydroorotate, the precursor to orotate and orotidylate (Iwahana, H. et al. (1996) Biochem. Biophys. Res. Commun. 219:249-255). Further steps then lead to the synthesis of uridine nucleotides from orotidylate. Cytidine nucleotides are derived from uridine-5′-triphosphate (UTP) by the amidation of UTP using glutamine as the amino donor and the enzyme CTP synthetase. Regulatory mutations in the human CTP synthetase are believed to confer multi-drug resistance to agents widely used in cancer therapy (Yamauchi, M. et al. (1990) EMBO J. 9:2095-2099).

Ligases forming carbon-carbon bonds include the carboxylases acetyl-CoA carboxylase and pyruvate carboxylase. Acetyl-CoA carboxylase catalyzes the carboxylation of acetyl-CoA from CO₂ and H₂O using the energy of ATP hydrolysis. Acetyl-CoA carboxylase is the rate-limiting step in the biogenesis of long-chain fatty acids. Two isoforms of acetyl-CoA carboxylase, types I and types II, are expressed in human in a tissue-specific manner (Ha, J. et al. (1994) Eur. J. Biochem. 219:297-306): Pyruvate carboxylase is a nuclear-encoded mitochondrial enzyme that catalyzes the conversion of pyruvate to oxaloacetate, a key intermediate in the citric acid cycle.

Ligases forming phosphoric ester bonds include the DNA ligases involved in both DNA replication and repair. DNA ligases seal phosphodiester bonds between two adjacent nucleotides in a DNA chain using the energy from ATP hydrolysis to first activate the free 5′-phosphate of one nucleotide and then react it with the 3′-OH group of the adjacent nucleotide. This resealing reaction is used in both DNA replication to join small DNA fragments called Okazaki fragments that are transiently formed in the process of replicating new DNA, and in DNA repair. DNA repair is the process by which accidental base changes, such as those produced by oxidative damage, hydrolytic attack, or uncontrolled methylation of DNA, are corrected before replication or transcription of the DNA can occur. Bloom's syndrome is an inherited human disease in which individuals are partially deficient in DNA ligation and consequently have an increased incidence of cancer (Alberts, B. et al. (1994) The Molecular Biology of the Cell, Garland Publishing Inc., New York N.Y., p. 247).

Molecules Associated with Growth and Development

Human growth and development requires the spatial and temporal regulation of cell differentiation, cell proliferation, and apoptosis. These processes coordinately control reproduction, aging, embryogenesis, morphogenesis, organogenesis, and tissue repair and maintenance. At the cellular level, growth and development is governed by the cell's decision to enter into or exit from the cell division cycle and by the cell's commitment to a terminally differentiated state. These decisions are made by the cell in response to extracellular signals and other environmental cues it receives. The following discussion focuses on the molecular mechanisms of cell division, reproduction, cell differentiation and proliferation, apoptosis, and aging.

Cell Division

Cell division is the fundamental process by which all living things grow and reproduce. In unicellular organisms such as yeast and bacteria, each cell division doubles the number of organisms, while in multicellular species many rounds of cell division are required to replace cells lost by wear or by programmed cell death, and for cell differentiation to produce a new tissue or organ Details of the cell division cycle may vary, but the basic process consists of three principle events. The first event, interphase, involves preparations for cell division, replication of the DNA, and production of essential proteins. In the second event, mitosis, the nuclear material is divided and separates to opposite sides of the cell. The final event, cytokinesis, is division and fission of the cell cytoplasm. The sequence and timing of cell cycle transitions is under the control of the cell cycle regulation system which controls the process by positive or negative regulatory circuits at various check points.

Regulated progression of the cell cycle depends on the integration of growth control pathways with the basic cell cycle machinery. Cell cycle regulators have been identified by selecting for human and yeast cDNAs that block or activate cell cycle arrest signals in the yeast mating pheromone pathway when they are overexpressed. Known regulators include human CPR (cell cycle progression restoration) genes, such as CPR8 and CPR2, and yeast CDC (cell division control) genes, including CDC91, that block the arrest signals. The CPR genes express a variety of proteins including cyclins, tumor suppressor binding proteins, chaperones, transcription factors, translation factors, and RNA-binding proteins (Edwards, M. C. et al.(1997) Genetics 147:1063-1076).

Several cell cycle transitions, including the entry and exit of a cell from mitosis, are dependent upon the activation and inhibition of cyclin-dependent kinases (Cdks). The Cdks are composed of a kinase subunit, Cdk, and an activating subunit, cyclin, in a complex that is subject to many levels of regulation. There appears to be a single Cdk in Saccharomyces cerevisiae and Saccharomyces pombe whereas mammals have a variety of specialized Cdks. Cyclins act by binding to and activating cyclin-dependent protein kinases which then phosphorylate and activate selected proteins involved in the mitotic process. The Cdk-cyclin complex is both positively and negatively regulated by phosphorylation, and by targeted degradation involving molecules such as CDC4 and CDC53. In addition, Cdks are further regulated by binding to inhibitors and other proteins such as Suc1 that modify their specificity or accessibility to regulators (Patra, D. and W. G. Dunphy (1996) Genes Dev. 10:1503-1515; and Mathias, N. et al. (1996) Mol. Cell Biol. 16:66346643).

Reproduction

The male and female reproductive systems are complex and involve many aspects of growth and development. The anatomy and physiology of the male and female reproductive systems are reviewed in (Guyton, A. C. (1991) Textbook of Medical Physiology, W. B. Saunders Co., Philadelphia Pa., pp. 899-928).

The male reproductive system includes the process of spermatogenesis, in which the sperm are formed, and male reproductive functions are regulated by various hormones and their effects on accessory sexual organs, cellular metabolism, growth, and other bodily functions.

Spermatogenesis begins at puberty as a result of stimulation by gonadotropic hormones released from the anterior pituitary. Immature sperm (spermatogonia) undergo several mitotic cell divisions before undergoing meiosis and full maturation. The testes secrete several male sex hormones, the most abundant being testosterone, that is essential for growth and division of the immature sperm, and for the masculine characteristics of the male body. Three other male sex hormones, gonadotropin-releasing hormone (GnRH), luteinizing hormone (LH), and follicle-stimulating hormone (FSH) control sexual function.

The uterus, ovaries, fallopian tubes, vagina, and breasts comprise the female reproductive system. The ovaries and uterus are the source of ova and the location of fetal development, respectively. The fallopian tubes and vagina are accessory organs attached to the top and bottom of the uterus, respectively. Both the uterus and ovaries have additional roles in the development and loss of reproductive capability during a female's lifetime. The primary role of the breasts is lactation Multiple endocrine signals from the ovaries, uterus, pituitary, hypothalamus, adrenal glands, and other tissues coordinate reproduction and lactation. These signals vary during the monthly menstruation cycle and during the female's lifetime. Similarly, the sensitivity of reproductive organs to these endocrine signals varies during the female's lifetime.

A combination of positive and negative feedback to the ovaries, pituitary and hypothalamus glands controls physiologic changes during the monthly ovulation and endometrial cycles. The anterior pituitary secretes two major gonadotropin hormones, follicle-stimulating hormone (FSH) and luteinizing hormone (LH), regulated by negative feedback of steroids, most notably by ovarian estradiol. If fertilization does not occur, estrogen and progesterone levels decrease. This sudden reduction of the ovarian hormones leads to menstruation, the desquamation of the endometrium.

Hormones further govern an the steps of pregnancy, parturition, lactation, and menopause. During pregnancy large quantities of human chorionic gonadotropin (hCG), estrogens, progesterone, and human chorionic somatomammotropin (hCS) are formed by the placenta. hCG, a glycoprotein similar to luteinizing hormone, stimulates the corpus luteum to continue producing more progesterone and estrogens, rather than to involute as occurs if the ovum is not fertilized hCS is similar to growth hormone and is crucial for fetal nutrition.

The female breast also matures during pregnancy. Large amounts of estrogen secreted by the placenta trigger growth and branching of the breast milk ductal system while lactation is initiated by the secretion of prolactin by the pituitary gland.

Parturition involves several hormonal changes that increase uterine contractility toward the end of pregnancy, as follows. The levels of estrogens increase more than those of progesterone. Oxytocin is secreted by the neurohypophysis. Concomitantly, uterine sensitivity to oxytocin increases. The fetus itself secretes oxytocin, cortisol (from adrenal glands), and prostaglandins.

Menopause occurs when most of the ovarian follicles have degenerated. The ovary then produces less estradiol, reducing the negative feedback on the pituitary and hypothalamus glands. Mean levels of circulating FSH and LH increase, even as ovulatory cycles continue. Therefore, the ovary is less responsive to gonadotropins, and there is an increase in the time between menstrual cycles.

Consequently, menstrual bleeding ceases and reproductive capability ends.

Cell Differentiation and Proliferation

Tissue growth involves complex and ordered patterns of cell proliferation, cell differentiation, and apoptosis. Cell proliferation must be regulated to maintain both the number of cells and their spatial organization. This regulation depends upon the appropriate expression of proteins which control cell cycle progression in response to extracellular signals, such as growth factors and other mitogens, and intracellular cues, such as DNA damage or nutrient starvation. Molecules which directly or indirectly modulate cell cycle progression fall into several categories, including growth factors and their receptors, second messenger and signal transduction proteins, oncogene products, tumor-suppressor proteins, and mitosis-promoting factors.

Growth factors were originally described as serum factors required to promote cell proliferation. Most growth factors are large, secreted polypeptides that act on cells in their local environment. Growth factors bind to and activate specific cell surface receptors and initiate intracellular signal transduction cascades. Many growth factor receptors are classified as receptor tyrosine kinases which undergo autophosphorylation upon ligand binding. Autophosphorylation enables the receptor to interact with signal transduction proteins characterized by the presence of SH2 or SH3 domains (Src homology regions 2 or 3). These proteins then modulate the activity state of small G-proteins, such as Ras, Rab, and Rho, along with GTPase activating proteins (GAPs), guanine nucleotide releasing proteins (GNRPs), and other guanine nucleotide exchange factors. Small G proteins act as molecular switches that activate other downstream events, such as mitogen-activated protein kinase (MAP kinase) cascades. MAP kinases ultimately activate transcription of mitosis-promoting genes.

In addition to growth factors, small signaling peptides and hormones also influence cell proliferation. These molecules bind primarily to another class of receptor, the trimeric G-protein coupled receptor (GPCR), found predominantly on the surface of immune, neuronal and neuroendocrine cells. Upon ligand binding, the GPCR activates a trimeric G protein which in turn triggers increased levels of intracellular second messengers such as phospholipase C, Ca2+, and cyclic AMP. Most GPCR-mediated signaling pathways indirectly promote cell proliferation by causing the secretion or breakdown of other signaling molecules that have direct mitogenic effects. These signaling cascades often involve activation of kinases and phosphatases. Some growth factors, such as some members of the transforming growth factor beta (TGF-β) family, act on some cells to stimulate cell proliferation and on other cells to inhibit it Growth factors may also stimulate a cell at one concentration and inhibit the same cell at another concentration. Most growth factors also have a multitude of other actions besides the regulation of cell growth and division: they can control the proliferation, survival, differentiation, migration, or function of cells depending on the circumstance. For example, the tumor necrosis factor/nerve growth factor (TNF/NGF) family can activate or inhibit cell death, as well as regulate proliferation and differentiation. The cell response depends on the type of cell, its stage of differentiation and transformation status, which surface receptors are stimulated, and the types of stimuli acting on the cell (Smith, A. et al. (1994) Cell 76:959-962; and Nocentini, G. et al. (1997) Proc. Natl. Acad. Sci. USA 94:6216-6221).

Neighboring cells in a tissue compete for growth factors, and when provided with “ited” quantities in a perfused system win grow to even higher cell densities before reaching density-dependent inhibition of cell division. Cells often demonstrate an anchorage dependence of cell division as well. This anchorage dependence may be associated with the formation of focal contacts linking the cytoskeleton with the extracellular matrix (ECM). The expression of ECM components can be stimulated by growth factors. For example, TGF-5 stimulates fibroblasts to produce a variety of ECM proteins, including fibronectin, collagen, and tenascin (Pearson, C. A. et al. (1988) EMBO J. 7:2677-2981). In fact, for some cell types specific ECM molecules, such as laminin or fibronectin, may act as growth factors. Tenascin-C and -R, expressed in developing and lesioned neural tissue, provide stimulatory/anti-adhesive or inhibitory properties, respectively, for axonal growth (Faissner, A (1997) Cell Tissue Res. 290:331-341).

Cancers are associated with the activation of oncogenes which are derived from normal cellular genes. These oncogenes encode oncoproteins which convert normal cells into malignant cells. Some oncoproteins are mutant isoforms of the normal protein, and other oncoproteins are abnormally expressed with respect to location or amount of expression The latter category of oncoprotein causes cancer by altering transcriptional control of cell proliferation. Five classes of oncoproteins are known to affect cell cycle controls. These classes include growth factors, growth factor receptors, intracellular signal transducers, nuclear transcription factors, and cell-cycle control proteins. Viral oncogenes are integrated into the human genome after infection of human cells by certain viruses. Examples of viral oncogenes include v-src, v-abl, and v-fps.

Many oncogenes have been identified and characterized These include v-src, erbA, erbB, her-2, mutated G_{S}, src, abl, ras, crk, jun, fos, myc, and mutated tumor-suppressor genes such as RB, p53, mdm2, Cip1, p16, and cyclin D. Transformation of normal genes to oncogenes may also occur by chromosomal translocation. The Philadelphia chromosome, characteristic of chronic myeloid leukemia and a subset of acute lymphoblastic leukemias, results from a reciprocal translocation between chromosomes 9 and 22 that moves a truncated portion of the proto-oncogene c-abl to the breakpoint cluster region (bcr) on chromosome 22.

Tumor-suppressor genes are involved in regulating cell proliferation. Mutations which cause reduced or loss of function in tumor-suppressor genes result in uncontrolled cell proliferation. For example, the retinoblastoma gene product (RB), in a non-phosphorylated state, binds several early-response genes and suppresses their transcription, thus blocking cell division. Phosphorylation of RB causes it to dissociate from the genes, releasing the suppression, and allowing cell division to proceed.

Apoptosis

Apoptosis is the genetically controlled process by which unneeded or defective cells undergo programmed cell death. Selective elimination of cells is as important for morphogenesis and tissue remodeling as is cell proliferation and differentiation. Lack of apoptosis may result in hyperplasia and other disorders associated with increased cell proliferation. Apoptosis is also a critical component of the immune response. Immune cells such as cytotoxic T-cells and natural killer cells prevent the spread of disease by inducing apoptosis in tumor cells and virus-infected cells. In addition, immune cells that fail to distinguish self molecules from foreign molecules must be eliminated by apoptosis to avoid an autoimmune response.

Apoptotic cells undergo distinct morphological changes. Hallmarks of apoptosis include cell shrinkage, nuclear and cytoplasmic condensation, and alterations in plasma membrane topology. Biochemically, apoptotic cells are characterized by increased intracellular calcium concentration, fragmentation of chromosomal DNA, and expression of novel cell surface components.

The molecular mechanisms of apoptosis are highly conserved, and many of the key protein regulators and effectors of apoptosis have been identified. Apoptosis generally proceeds in response to a signal which is transduced intracellularly and results in altered patterns of gene expression and protein activity. Signaling molecules such as hormones and cytokines are known both to stimulate and to inhibit apoptosis through interactions with cell surface receptors. Transcription factors also play an important role in the onset of apoptosis. A number of downstream effector molecules, particularly proteases such as the cysteine proteases called caspases, have been implicated in the degradation of cellular components and the proteolytic activation of other apoptotic effectors.

Aging and Senescence

Studies of the aging process or senescence have shown a member of characteristic cellular and molecular changes (Fauci et al. (1998) Harrison's Principles of Internal Medicine, McGraw-Hill, New York N.Y., p.37). These characteristics include increases in chromosome structural abnormalities, DNA cross-linking, incidence of single-stranded breaks in DNA, losses in DNA methylation, and degradation of telomere regions. In addition to these DNA changes, post-translational alterations of proteins increase including, deamidation, oxidation, cross-linking, and nonenzymatic glycation. Still further molecular changes occur in the mitochondria of aging cells through deterioration of structure. These changes eventually contribute to decreased function in every organ of the body.

Biochemical Pathway Molecules

Biochemical pathways are responsible for regulating metabolism, growth and development, protein secretion and trafficking, environmental responses, and ecological interactions including immune response and response to parasites.

DNA Replication

Deoxyribonucleic acid (DNA), the genetic material, is found in both the nucleus and mitochondria of human cells. The bulk of human DNA is nuclear, in the form of linear chromosomes, while mitochondrial DNA is circular. DNA replication begins at specific sites called origins of replication. Bidirectional synthesis occurs from the origin via two growing forks that move in opposite directions. Replication is semi-conservative, with each daughter duplex containing one old strand and its newly synthesized complementary partner. Proteins involved in DNA replication include DNA polymerases, DNA primase, telomerase, DNA helicase, topoisomerases, DNA ligases, replication factors, and DNA-binding proteins.

DNA Recombination and Repair

Cells are constantly faced with replication errors and environmental assault (such as ultraviolet irradiation) that can produce DNA damage. Damage to DNA consists of any change that modifies the structure of the molecule. Changes to DNA can be divided into two general classes, single base changes and structural distortions. Any damage to DNA can produce a mutation, and the mutation may produce a disorder, such as cancer.

Changes in DNA are recognized by repair systems within the cell. These repair systems act to correct the damage and thus prevent any deleterious affects of a mutational event Repair systems can be divided into three general types, direct repair, excision repair, and retrieval systems. Proteins involved in DNA repair include DNA polymerase, excision repair proteins, excision and cross link repair proteins, recombination and repair proteins, RAD51 proteins, and BLN and WRN proteins that are homologs of RecQ helicase. When the repair systems are eliminated, cells become exceedingly sensitive to environmental mutagens, such as ultraviolet irradiation Patients with disorders associated with a loss in DNA repair systems often exhibit a high sensitivity to environmental mutagens. Examples of such disorders include xeroderma pigmentosum (XP), Bloom's syndrome (BS), and Werner's syndrome (WS) (Yamagata, K et al. (1998) Proc. Natl. Acad. Sci. USA 95:8733-8738), ataxia telangiectasia, Cockayne's syndrome, and Fanconi's anemia.

Recombination is the process whereby new DNA sequences are generated by the movements of large pieces of DNA. In homologous recombination, which occurs during meiosis and DNA repair, parent DNA duplexes align at regions of sequence similarity, and new DNA molecules form by the breakage and joining of homologous segments. Proteins involved include RAD51 recombinase. In site-specific recombination, two specific but not necessarily homologous DNA sequences are exchanged. In the immune system this process generates a diverse collection of anitibody and T cell receptor genes. Proteins involved in site-specific recombination in the immune system include recombination activating genes 1 and 2 (RAG1 and RAG2). A defect in immune system site-specific recombination causes severe combined immunodeficiency disease in mice.

RNA Metabolism

Ribonucleic acid (RNA) is a linear single-stranded polymer of four nucleotides, ATP, CTP, UTP, and GTP. In most organisms, RNA is transcribed as a copy of DNA, the genetic material of the organism. In retroviruses RNA rather than DNA serves as the genetic material. RNA copies of the genetic material encode proteins or serve various structural, catalytic, or regulatory roles in organisms. RNA is classified according to its cellular localization and function Messenger RNAs (mRNAs) encode polypeptides. Ribosomal RNAs (rRNAs) are assembled, along with ribosomal proteins, into ribosomes, which are cytoplasmic particles that translate mRNA into polypeptides. Transfer RNAs (tRNAs) are cytosolic adaptor molecules that function in mRNA translation by recognizing both an mRNA codon and the amino acid that matches that codon. Heterogeneous nuclear RNAs (hnRNAs) include mRNA precursors and other nuclear RNAs of various sizes. Small nuclear RNAs (snRNAs) are a part of the nuclear spliceosome complex that removes intervening, non-coding sequences (introns) and rejoins exons in pre-mRNAs.

RNA Transcription

The transcription process synthesizes an RNA copy of DNA. Proteins involved include multi-subunit RNA polymerases, transcription factors IIA, IIB, IID, IIE, IIF, IIH, and IIJ. Many transcription factors incorporate DNA-binding structural motifs which comprise either α-helices or β-sheets that bind to the major groove of DNA. Four well-characterized structural motifs are helix-turn-helix, zinc finger, leucine zipper, and helix-loop-helix.

RNA Processing

Various proteins are necessary for processing of transcribed RNAs in the nucleus. Pre-mRNA processing steps include capping at the 5′ end with methylguanosine, polyadenylating the 3′ end, and splicing to remove introns. The spliceosomal complex is comprised of five small nuclear ribonucleoprotein particles (snRNPs) designated U1, U2, U4, U5, and U6. Each snRNP contains a single species of snRNA and about ten proteins. The RNA components of some snRNPs recognize and base-pair with intron consensus sequences. The protein components mediate spliceosome assembly and the splicing reaction. Autoantibodies to snRNP proteins are found in the blood of patients with systemic lupus erythematosus (Stryer, L. (1995) Biochemistry W. H. Freeman and Company, New York N.Y., p. 863).

Heterogeneous nuclear ribonucleoproteins (hnRNPs) have been identified that have roles in splicing, exporting of the mature RNAs to the cytoplasm, and mRNA translation (Biamonti, G. et al. (1998) Clin Exp. Rheumatol. 16:317-326). Some examples of hnRNPs include the yeast proteins Hrplp, involved in cleavage and polyadenylation at the 3′ end of the RNA; Cbp80p, involved in capping the 5′ end of the RNA; and Npl3p, a homolog of mammalian hnRNP A1, involved in export of mRNA from the nucleus (Shen, E. C. et al. (1998) Genes Dev. 12:679-691). HnRNPs have been shown to be important targets of the autoimmune response in rheumatic diseases (Biamonti, supra).

Many snRNP proteins, and alternative splicing factors are characterized by an RNA recognition motif (RRM). (Reviewed in Birney, E. et al. (1993) Nucleic Acids Res. 21:5803-5816.) The RRM is about 80 amino acids in length and forms four β-strands and two α-helices arranged in an a/0 sandwich. The RRM contains a core RNP-1 octapeptide motif along with surrounding conserved sequences.

RNA Stability and Degradation

RNA helicases alter and regulate RNA conformation and secondary structure by using energy derived from ATP hydrolysis to destabilize and unwind RNA duplexes. The most well-characterized and ubiquitous family of RNA helicases is the DEAD-box family, so named for the conserved B-type ATP-binding motif which is diagnostic of proteins in this family. Over 40 DEAD-box helicases have been identified in organisms as diverse as bacteria, insects, yeast, amphibians, mammals, and plants. DEAD-box helicases function in diverse processes such as translation initiation, splicing, ribosome assembly, and RNA editing, transport, and stability. Some DEAD-box helicases play tissue- and stage-specific roles in spermatogenesis and embryogenesis. (Reviewed in Linder, P. et al. (1989) Nature 337:121-122.)

Overexpression of the DEAD-box 1 protein (DDX1) may play a role in the progression of neuroblastoma (Nb) and retinoblastoma (Rb) tumors. Other DEAD-box helicases have been implicated either directly or indirectly in ultraviolet light-induced tumors, B cell lymphoma, and myeloid malignancies. (Reviewed in Godbout, R. et al. (1998) J. Biol. Chem. 273:21161-21168.)

Ribonucleases (RNases) catalyze the hydrolysis of phosphodiester bonds in RNA chains, thus cleaving the RNA. For example, RNase P is a ribonucleoprotein enzyme which cleaves the 5′ end of pre-tRNAs as part of their maturation process. RNase H digests the RNA strand of an RNA/DNA hybrid. Such hybrids occur in cells invaded by retroviruses, and RNase H is an important enzyme in the retroviral replication cycle. RNase H domains are often found as a domain associated with reverse transcriptases. RNase activity in serum and cell extracts is elevated in a variety of cancers and infectious diseases (Schein, C. H. (1997) Nat. Biotechnol. 15:529-536). Regulation of RNase activity is being investigated as a means to control tumor angiogenesis, allergic reactions, viral infection and replication, and fungal infections.

Protein Translation

The eukaryotic ribosome is composed of a 60S (large) subunit and a 40S (small) subunit, which together form the 80S ribosome. In addition to the 18S, 28S, 5S, and 5.8S rRNAs, the ribosome also contains more than fifty proteins. The ribosomal proteins have a prefix which denotes the subunit to which they belong, either L (large) or S (small). Three important sites are identified on the ribosome. The aminoacyl-tRNA site (A site) is where charged tRNAs (with the exception of the initiator-tRNA) bind on arrival at the ribosome. The peptidyl-tRNA site (P site) is where new peptide bonds are formed, as well as where the initiator tRNA binds. The exit site (E site) is where deacylated tRNAs bind prior to their release from the ribosome. (Translation is reviewed in Stryer, L. (1995) Biochemistry, W. H. Freeman and Company, New York N.Y., pp. 875-908; and Lodish, H. et al. (1995) Molecular Cell Biology, Scientific American Books, New York N.Y., pp. 119-138.)

tRNA Charging

Protein biosynthesis depends on each amino acid forming a linkage with the appropriate tRNA The aminoacyl-tRNA synthetases are responsible for the activation and correct attachment of an amino acid with its cognate tRNA The 20 aminoacyl-tRNA synthetase enzymes can be divided into two structural classes, Class I and Class II. Autoantibodies against aminoacyl-tRNAs are generated by patients with dermatomyositis and polymyositis, and correlate strongly with complicating interstitial lung disease (ILD). These antibodies appear to be generated in response to viral infection, and coxsackie virus has been used to induce experimental viral myositis in animals.

Translation Initiation

Initiation of translation can be divided into three stages. The first stage brings an initiator transfer RNA (Met-tRNA) together with the 40S ribosomal subunit to form the 43S preinitiation complex. The second stage binds the 43S preinitiation complex to the mRNA, followed by migration of the complex to the correct AUG initiation codon. The third stage brings the 60S ribosomal subunit to the 40S subunit to generate an 80S ribosome at the initiation codon. Regulation of translation primarily involves the first and second stage in the initiation process (Pain, V. M. (1996) Eur. J. Biochem. 236:747-771).

Several initiation factors, many of which contain multiple subunits, are involved in bringing an initiator tRNA and 40S ribosomal subunit together. eIF2, a guanine nucleotide binding protein, recruits the initiator tRNA to the 40S ribosomal subunit. Only when eIF2 is bound to GTP does it associate with the initiator tRNA eIF2B, a guanine nucleotide exchange protein, is responsible for converting eIF2 from the GDP-bound inactive form to the GTP-bound active form. Two other factors, eIF1A and eIF3 bind and stabilize the 40S subunit by interacting with 18S ribosomal RNA and specific ribosomal structural proteins. eIF3 is also involved in association of the 40S ribosomal subunit with mRNA. The Met-tRNA_{f}, eIF1A, eIF3, and 40S ribosomal subunit together make up the 43S preinitiation complex (Pain, supra).

Additional factors are required for binding of the 43S preinitiation complex to an mRNA molecule, and the process is regulated at several levels. eIF4F is a complex consisting of three proteins: eIF4E, eIF4A, and eIF4G. eIF4E recognizes and binds to the mRNA 5′-terminal m⁷GTP cap, eIF4A is a bidirectional RNA-dependent helicase, and eIF4G is a scaffolding polypeptide. eIF4G has three binding domains. The N-terminal third of eIF4G interacts with eIF4E, the central third interacts with eIF4A, and the C-terminal third interacts with eIF3 bound to the 43S preinitiation complex. Thus, eIF4G acts as a bridge between the 40S ribosomal subunit and the mRNA (Hentze, M. W. (1997) Science 275:500-501).

The ability of eIF4F to initiate binding of the 43S preinitiation complex is regulated by structural features of the mRNA The mRNA molecule has an untranslated region (UM) between the 5, cap and the AUG start codon. In some mRNAs this region forms secondary structures that impede binding of the 43S preinitiation complex. The helicase activity of eIF4A is thought to function in removing this secondary structure to facilitate binding of the 43S preinitiation complex (Pain, supra).

Translation Elongation

Elongation is the process whereby additional amino acids are joined to the initiator methionine to form the complete polypeptide chain. The elongation factors EF1α, EF1βγ, and EF2 are involved in elongating the polypeptide chain following initiation. EF1α is a GTP-binding protein. InEFla's GTP-bound form, it brings an aminoacyl-tRNA to the ribosome's A site. The amino acid attached to the newly arrived aminoacyl-tRNA forms a peptide bond with the initiator methionine. The GTP on EF1α is hydrolyzed to GDP, and EF1α-GDP dissociates from the ribosome. EF1βγ binds EF1α-GDP and induces the dissociation of GDP from EF1α, allowing EF1α to bind GTP and a new cycle to begun

As subsequent aminoacyl-tRNAs are brought to the ribosome, EF-G, another GTP-binding protein, catalyzes the translocation of tRNAs from the A site to he P site and finally to the E site of the ribosome. This allows the processivity of translation.

Translation Termination

The release factor eRF carries out termination of translation. eRF recognizes stop codons in the mRNA, leading to the release of the polypeptide chain from the ribosome.

Post-Translational Pathways

Proteins may be modified after translation by the addition of phosphate, sugar, prenyl, fatty acid, and other chemical groups. These modifications are often required for proper protein activity. Enzymes involved in post-translational modification include kinases, phosphatases, glycosyltransferases, and prenyltransferases. The conformation of proteins may also be modified after translation by the introduction and rearrangement of disulfide bonds (rearrangement catalyzed by protein disulfide isomerase), the isomerization of proline sidechains by prolyl isomerase, and by interactions with molecular chaperone proteins.

Proteins may also be cleaved by proteases. Such cleavage may result in activation, inactivation, or complete degradation of the protein. Proteases include serine proteases, cysteine proteases, aspartic proteases, and metalloproteases. Signal peptidase in the endoplasmic reticulum (ER) lumen cleaves the signal peptide from membrane or secretory proteins that are imported into the ER. Ubiquitin proteases are associated with the ubiquitin conjugation system (UCS), a major pathway for the degradation of cellular proteins in eukaryotic cells and some bacteria. The UCS mediates the elimination of abnormal proteins and regulates the half-lives of important regulatory proteins that control cellular processes such as gene transcription and cell cycle progression. In-the UCS pathway, proteins targeted for degradation are conjugated to a ubiquitin, a small heat stable protein. Proteins involved in the UCS include ubiquitin-activating enzyme, ubiquitin-conjugating enzymes, ubiquitin-ligases, and ubiquitin C-terminal hydrolases. The ubiquitinated protein is then recognized and degraded by the proteasome, a large, multisubunit proteolytic enzyme complex, and ubiquitin is released for reutilization by ubiquitin protease.

Lipid Metabolism

Lipids are water-insoluble, oily or greasy substances that are soluble in nonpolar solvents such as chloroform or ether. Neutral fats (triacylglycerols) serve as major fuels and energy stores. Polar lipids, such as phospholipids, sphingolipids, glycolipids, and cholesterol, are key structural components of cell membranes.

Lipid metabolism is involved in human diseases and disorders. In the arterial disease atherosclerosis, fatty lesions form on the inside of the arterial wall. These lesions promote the loss of arterial flexibility and the formation of blood clots (Guyton, A. C. Textbook of Medical Physiology (1991) W. B. Saunders Company, Philadelphia Pa., pp.760-763). In Tay-Sachs disease, the GM₂ ganglioside (a sphingolipid) accumulates in lysosomes of the central nervous system due to a lack of the enzyme N-acetylhexosaminidase. Patients suffer nervous system degeneration leading to early death (Fauci, AS. et al. (1998) Harrison's Principles of Internal Medicine McGraw-Hill, New York N.Y., p. 2171). The Niemann-Pick diseases are caused by defects in lipid metabolism. Niemann-Pick diseases types A and B are caused by accumulation of sphingomyelin (a sphingolipid) and other lipids in the central nervous system due to a defect in the enzyme sphingomyelinase, leading to neurodegeneration and lung disease. Niemann-Pick disease type C results from a defect in cholesterol transport, leading to the accumulation of sphingomyelin and cholesterol in lysosomes and a secondary reduction in sphingomyelinase activity. Neurological symptoms such as grand mal seizures, ataxia, and loss of previously learned speech, manifest 1-2 years after birth. A mutation in the NPC protein, which contains a putative cholesterol-sensing domain, was found in a mouse model of Niemann-Pick disease type C (Fauci, supra, p. 2175; Loftus, S. K et al. (1997) Science 277:232-235). (Lipid metabolism is reviewed in Stryer, L. (1995) Biochemistry, W. H. Freeman and Company, New York N.Y.; Lehninger, A (1982) Principles of Biochemistry Worth Publishers, Inc., New York N.Y.; and ExPASy “Biochemical Pathways” index of Boehringer Mannheim World Wide Web site.)

Fatty Acid Synthesis

Fatty acids are long-chain organic acids with a single carboxyl group and a long non-polar hydrocarbon tail. Long-chain fatty acids are essential components of glycolipids, phospholipids; and cholesterol, which are building blocks for biological membranes, and of triglycerides, which are biological fuel molecules. Long-chain fatty acids are also substrates for eicosanoid production, and are important in the functional modification of certain complex carbohydrates and proteins. 16-carbon and 18-carbon fatty acids are the most common.

Fatty acid synthesis occurs in the cytoplasm. In the first step, acetyl-Coenzyme A (CoA) carboxylase (ACC) synthesizes malonyl-CoA from acetyl-CoA and bicarbonate. The enzymes which catalyze the remaining reactions are covalently linked into a single polypeptide chain, referred to as the multifunctional enzyme fatty acid synthase (FAS). FAS catalyzes the synthesis of palmitate from acetyl-CoA and malonyl-CoA FAS contains acetyl transferase, malonyl transferase, β-ketoacetyl synthase, acyl carrier protein, β-ketoacyl reductase, dehydratase, enoyl reductase, and thioesterase activities. The final product of the FAS reaction is the 16 carbon fatty acid palmitate. Further elongation, as well as unsaturation, of palmitate by accessory enzymes of the ER produces the variety of long chain fatty acids required by the individual cell. These enzymes include a NADH-cytocbrome b₅ reductase, cytochrome b₅, and a desaturase.

Phospholipid and Triacylglcerol Synthesis

Triacylglycerols, also known as triglycerides and neutral fats, are major energy stores in animals. Triacylglycerols are esters of glycerol with three fatty acid chains. Glycerol-3-phosphate is produced from dihydroxyacetone phosphate by the enzyme glycerol phosphate dehydrogenase or from glycerol by glycerol kinase. Fatty acid-CoA's are produced from fatty acids by fatty acyl-CoA synthetases. Glyercol-3-phosphate is acylated with two fatty acyl-CoA's by the enzyme glycerol phosphate acyltransferase to give phosphatidate. Phosphatidate phosphatase converts phosphatidate to diacylglycerol, which is subsequently acylated to a triacylglyercol by the enzyme diglyceride acyltransferase. Phosphatidate phosphatase and diglyceride acyltransferase form a triacylglyerol synthease complex bound to the ER membrane.

A major class of phospholipids are the phosphoglycerides, which are composed of a glycerol backbone, two fatty acid chains, and a phosphorylated alcohol. Phosphoglycerides are components of cell membranes. Principal phosphoglycerides are phosphatidyl choline, phosphatidyl ethanolamine, phosphatidyl serine, phosphatidyl inositol, and diphosphatidyl glycerol. Many enzymes involved in phosphoglyceride synthesis are associated with membranes (Meyers, R. A. (1995) Molecular Biology and Biotechnology, VCH Publishers Inc., New York N.Y., pp. 494-501). Phosphatidate is converted to CDP-diacylglycerol by the enzyme phosphatidate cytidylyltransferase (ExPASy ENZYME EC 2.7.7.41). Transfer of the diacylglycerol group from CDP-diacylglycerol to serine to yield phosphatidyl serine, or to inositol to yield phosphatidyl inositol, is catalyzed by the enzymes CDP-diacylglycerol-serine O-phosphatidyltransferase and CDP-diacylglycerol-inositol 3-phosphatidyltransferase, respectively (ExPASy ENZYME EC 2.7.8.8; ExPASy ENZYME EC 2.7.8.11). The enzyme phosphatidyl serine decarboxylase catalyzes the conversion of phosphatidyl serine to phosphatidyl ethanolamine, using a pyruvate cofactor (Voelker, D. R. (1997) Biochim. Biophys. Acta 1348:236-244).

Phosphatidyl choline is formed using diet-derived choline by the reaction of CDP-choline with 1,2-diacylglycerol, catalyzed by diacylglycerol cholinephosphotransferase (ExPASy ENZYME 2.7.8.2).

Sterol, Steroid, and Isoprenoid Metabolism

Cholesterol, composed of four fused hydrocarbon rings with an alcohol at one end, moderates the fluidity of membranes in which it is incorporated. In addition, cholesterol is used in the synthesis of steroid hormones such as cortisol, progesterone, estrogen, and testosterone. Bile salts derived from cholesterol facilitate the digestion of lipids. Cholesterol in the skin forms a barrier that prevents excess water evaporation from the body. Farnesyl and geranylgeranyl groups, which are derived from cholesterol biosynthesis intermediates, are post-translationally added to signal transduction proteins such as ras and protein-targeting proteins such as rab. These modifications are important for the activities of these proteins (Guyton, supra; Stryer, supra, pp. 279-280, 691-702, 934).

Mammals obtain cholesterol derived from both de novo biosynthesis and the diet. The liver is the major site of cholesterol biosynthesis in mammals. Two acetyl-CoA molecules initially condense to form acetoacetyl-CoA, catalyzed by a thiolase. Acetoacetyl-CoA condenses with a third acetyl-CoA to form hydroxymethylglutaryl-CoA (HMG-CoA), catalyzed by HMG-CoA synthase. Conversion of HMG-COA to cholesterol is accomplished via a series of enzymatic steps known as the mevalonate pathway. The rate-limiting step is the conversion of HMG-CoA to mevalonate by HMG-CoA reductase. The drug lovastatin, a potent inhibitor of HMG-CoA reductase, is given to patients to reduce their serum cholesterol levels. Other mevalonate pathway enzymes include mevalonate kinase, phosphomevalonate kinase, diphosphomevalonate decarboxylase, isopentenyldiphosphate isomerase, dimethylallyl transferase, geranyl transferase, farnesyl-diphosphate farnesyltransferase, squalene monooxygenase, lanosterol synthase, lathosterol oxidase, and 7-dehydrocholesterol reductase.

Cholesterol is used in the synthesis of steroid hormones such as cortisol, progesterone, aldosterone, estrogen, and testosterone. First, cholesterol is converted to pregnenolone by cholesterol monooxygenases. The other steroid hormones are synthesized from pregnenolone by a series of enzyme-catalyzed reactions including oxidations, isomerizations, hydroxylations, reductions, and demethylations. Examples of these enzymes include steroid Δ-isomerase, 3β-hydroxy-Δ⁵-steroid dehydrogenase, steroid 21-monooxygenase, steroid 19-hydroxylase, and 3β-hydroxysteroid dehydrogenase. Cholesterol is also the precursor to vitamin D.

Numerous compounds contain 5-carbon isoprene units derived from the mevalonate pathway intermediate isopentenyl pyrophosphate. Isoprenoid groups are found in vitamin K, ubiquinone, retinal, dolichol phosphate (a carrier of oligosaccharides needed for N-lined glycosylation), and farnesyl and geranylgeranyl groups that modify proteins. Enzymes involved include farnesyl transferase, polyprenyl transferases, dolichyl phosphatase, and dolichyl kinase.

Sphingolipid Metabolism

Sphingolipids are an important class of membrane lipids that contain sphingosine, a long chain amino alcohol. They are composed of one long-chain fatty acid, one polar head alcohol, and sphingosine or sphingosine derivative. The three classes of sphingolipids are sphingomyelins, cerebrosides, and gangliosides. Sphingomyelins, which contain phosphocholine or phosphoethanolamine as their head group, are abundant in the myelin sheath surrounding nerve cells. Galactocerebrosides, which contain a glucose or galactose head group, are characteristic of the brain. Other cerebrosides are found in nonneural tissues. Gangliosides, whose head groups contain multiple sugar units, are abundant in the brain, but are also found in nonneural tissues.

Sphingolipids are built on a sphingosine backbone. Sphingosine is acylated to ceramide by the enzyme sphingosine acetyltransferase. Ceramide and phosphatidyl choline are converted to sphingomyelin by the enzyme ceramide choline phosphotransferase. Cerebrosides are synthesized by the linkage of glucose or galactose to ceramide by a transferase. Sequential addition of sugar residues to ceramide by transferase enzymes yields gangliosides.

Eicosanoid Metabolism

Eicosanoids, including prostaglandins, prostacyclin, thromboxanes, and leukotrienes, are 20-carbon molecules derived from fatty acids. Eicosanoids are signaling molecules which have roles in pain, fever, and inflammation. The precursor of all eicosanoids is arachidonate, which is generated from phospholipids by phospholipase A₂ and from diacylglycerols by diacylglycerol lipase. Leukotrienes are produced from arachidonate by the action of lipoxygenases. Prostaglandin synthase, reductases, and isomerases are responsible for the synthesis of the prostaglandins. Prostaglandins have roles in inflammation, blood flow, ion transport, synaptic transmission, and sleep. Prostacyclin and the thromboxanes are derived from a precursor prostaglandin by the action of prostacyclin synthase and thromboxane synthases, respectively.

Ketone Body Metabolism

Pairs of acetyl-CoA molecules derived from fatty acid oxidation in the liver can condense to form acetoacetyl-CoA, which subsequently forms acetoacetate, D-3-hydroxybutyrate, and acetone. These three products are known as ketone bodies. Enzymes involved in ketone body metabolism include HMG-COA synthetase, HMG-CoA cleavage enzyme, D-3-hydroxybutyrate dehydrogenase, acetoacetate decarboxylase, and 3-ketoacyl-CoA transferase. Ketone bodies are a normal fuel supply of the heart and renal cortex. Acetoacetate produced by the liver is transported to cells where the acetoacetate is converted back to acetyl-CoA and enters the citric acid cycle. In times of starvation, ketone bodies produced from stored triacylglyerols become an important fuel source, especially for the brain Abnormally high levels of ketone bodies are observed in diabetics. Diabetic coma can result if ketone body levels become too great

Lipid Mobilization

Within cells, fatty acids are transported by cytoplasmic fatty acid binding proteins (Online Mendelian Inheritance in Man (OMIM)*134650 Fatty Acid-Binding Protein 1, Liver; FABP1). Diazepam binding inhibitor (DBI), also known as endozepine and acyl CoA-binding protein, is an endogenous γ-aminobutyric acid (GABA) receptor ligand which is thought to down-regulate the effects of GABA DBI binds medium- and long-chain acyl-CoA esters with very high affinity and may function as an intracellular carrier of acyl-CoA esters (OMIM*125950 Diazepam Binding inhibitor; DBI; PROSITE PDOC00686 Acyl-CoA-binding protein signature).

Fat stored in liver and adipose triglycerides may be released by hydrolysis and transported in the blood Free fatty acids are transported in the blood by albumin Triacylglycerols and cholesterol esters in the blood are transported in lipoprotein particles. The particles consist of a core of hydrophobic lipids surrounded by a shell of polar lipids and apolipoproteins. The protein components serve in the solubilization of hydrophobic lipids and also contain cell-targeting signals. Lipoproteins include chylomicrons, chylomicron remnants, very-low-density lipoproteins (VLDL), intermediate-density lipoproteins (IDL), low-density lipoproteins (LDL), and high-density lipoproteins (DL). There is a strong inverse correlation between the levels of plasma HDL and risk of premature coronary heart disease.

Triacylglycerols in chylomicrons and VLDL are hydrolyzed by lipoprotein lipases that line blood vessels in muscle and other tissues that use fatty acids. Cell surface LDL receptors bind LDL particles which are then internalized by endocytosis. Absence of the LDL receptor, the cause of the disease familial hypercholesterolemia, leads to increased plasma cholesterol levels and ultimately to atherosclerosis. Plasma cholesteryl ester transfer protein mediates the transfer of cholesteryl esters from HDL to apolipoprotein B-containing lipoproteins. Cholesteryl ester transfer protein is important in the reverse cholesterol transport system and may play a role in atherosclerosis (Yamashita, S. et al. (1997) Curr. Opin. Lipidol. 8:101-110). Macrophage scavenger receptors, which bind and internalize modified lipoproteins, play a role in lipid transport and may contribute to atherosclerosis (Greaves, D. R. et al. (1998) Curr. Opin. Lipidol. 9:425-432).

Proteins involved in cholesterol uptake and biosynthesis are tightly regulated in response to cellular cholesterol levels. The sterol regulatory element binding protein (SREBP) is a sterol-responsive transcription factor. Under normal cholesterol conditions, SREBP resides in the ER membrane. When cholesterol levels are low, a regulated cleavage of SREBP occurs which releases the extracellular domain of the protein. This cleaved domain is then transported to the nucleus where it activates the transcription of the LDL receptor gene, and genes encoding enzymes of cholesterol synthesis, by binding the sterol regulatory element (SRE) upstream of the genes (Yang, J. et al. (1995) J. Biol. Chem. 270:12152-12161). Regulation of cholesterol uptake and biosynthesis also occurs via the oxysterol-binding protein (OSBP). OSBP is a high-affinity intracellular receptor for a variety of oxysterols that down-regulate cholesterol synthesis and stimulate cholesterol esterification (Lagace, T. A et al. (1997) Biochem. J. 326:205-213).

Beta-oxidation

Mitochondrial and peroxisomal beta-oxidation enzymes degrade saturated and unsaturated fatty acids by sequential removal of two-carbon units from CoA-activated fatty acids. The main beta-oxidation pathway degrades both saturated and unsaturated fatty acids while the auxiliary pathway performs additional steps required for the degradation of unsaturated fatty acids.

The pathways of mitochondrial and peroxisomal beta-oxidation use similar enzymes, but have different substrate specificities and functions. Mitochondria oxidize short-, medium-, and long-chain fatty acids to produce energy for cells. Mitochondrial beta-oxidation is a major energy source for cardiac and skeletal muscle. In liver, it provides ketone bodies to the peripheral circulation when glucose levels are low as in starvation, endurance exercise, and diabetes (Eaton, S. et al. (1996) Biochem. J. 320:345-357). Peroxisomes oxidize medium-, long-, and very-long-chain fatty acids, dicarboxylic fatty acids, branched fatty acids, prostaglandins, xenobiotics, and bile acid intermediates. The chief roles of peroxisomal beta-oxidation are to shorten toxic lipophilic carboxylic acids to facilitate their excretion and to shorten very-long-chain fatty acids prior to mitochondrial beta-oxidation (Mannaerts, G. P. and P. P. van Veldhoven (1993) Biochimie 75:147-158).

Enzymes involved in beta-oxidation include acyl CoA synthetase, carnitine acyltransferase, acyl CoA dehydrogenases, enoyl CoA hydratases, L-3-hydroxyacyl CoA dehydrogenase, β-ketothiolase, 2,4-dienoyl CoA reductase, and isomerase.

Lipid Cleavage and Degradation

Triglycerides are hydrolyzed to fatty acids and glycerol by lipases. Lysophospholipases (LPLs) are widely distributed enzymes that metabolize intracellular lipids, and occur in numerous isoforms. Small isoforms, approximately 15-30 kD, function as hydrolases; large isoforms, those exceeding 60 kD, function both as hydrolases and transacylases. A particular substrate for LPLS, lysophosphatidylcholine, causes lysis of cell membranes when it is formed or imported into a cell. LPLs are regulated by lipid factors including acylcarnitine, arachidonic acid, and phosphatidic acid. These lipid factors are signaling molecules important in numerous pathways, including the inflammatory response. (Anderson, R. et al. (1994) Toxicol. Appl. Pharmacol. 125:176-183; Selle, H. et al. (1993); Eur. J. Biochem. 212:411-416.)

The secretory phospholipase A₂ (PLA2) superfamily comprises a number of heterogeneous enzymes whose common feature is to hydrolyze the sn-2 fatty acid acyl ester bond of phosphoglycerides. Hydrolysis of the glycerophospholipids releases free fatty acids and lysophospholipids. PLA2 activity generates precursors for the biosynthesis of biologically active lipids, hydroxy fatty acids, and platelet-activating factor. PLA2 hydrolysis of the sn-2 ester bond in phospholipids generates free fatty acids, such as arachidonic acid and lysophospholipids.

Carbon and Carbohydrate Metabolism Carbohydrates, including sugars or saccharides, starch, and cellulose, are aldehyde or ketone compounds with multiple hydroxyl groups. The importance of carbohydrate metabolism is demonstrated by the sensitive regulatory system in place for maintenance of blood glucose levels. Two pancreatic hormones, insulin and glucagon, promote increased glucose uptake and storage by cells, and increased glucose release from cells, respectively. Carbohydrates have three important roles in mammalian cells. First, carbohydrates are used as energy stores, fuels, and metabolic intermediates. Carbohydrates are broken down to form energy in glycolysis and are stored as glycogen for later use. Second, the sugars deoxyribose and ribose form part of the structural support of DNA and RNA, respectively. Third, carbohydrate modifications are added to secreted and membrane proteins and lipids as they traverse the secretory pathway. Cell surface carbohydrate-containing macromolecules, including glycoproteins, glycolipids, and transmembrane proteoglycans, mediate adhesion with other cells and with components of the extracellular matrix. The extracellular matrix is comprised of diverse glycoproteins, glycosaminoglycans (GAGs), and carbohydrate-binding proteins which are secreted from the cell and assembled into an organized meshwork in close association with the cell surface. The interaction of the cell with the surrounding matrix profoundly influences cell shape, strength, flexibility, motility, and adhesion. These dynamic properties are intimately associated with signal transduction pathways controlling cell proliferation and differentiation, tissue construction, and embryonic development.

Carbohydrate metabolism is altered in several disorders including diabetes mellitus, hyperglycemia, hypoglycemia, galactosemia, galactokinase deficiency, and UDP-galactose4-epimerase deficiency (Fauci, A. S. et al. (1998) Harrison's Principles of Internal Medicine, McGraw-Hill New York N.Y., pp. 2208-2209). Altered carbohydrate metabolism is associated with cancer. Reduced GAG and proteoglycan expression is associated with human lung carcinomas (Nackaerts, K. et al. (1997) Int. J. Cancer 74:335-345). The carbohydrate determinants sialyl Lewis A and sialyl Lewis X are frequently expressed on human cancer cells (Kannagi, R. (1997) Glycoconj. J. 14:577-584). Alterations of the N-linked carbohydrate core structure of cell surface glycoproteins are linked to colon and pancreatic cancers (Schwarz, R. E. et al. (1996) Cancer Lett. 107:285-291). Reduced expression of the Sda blood group carbohydrate structure in cell surface glycolipids and glycoproteins is observed in gastrointestinal cancer (Dohi, T. et al. (1996) Int. J. Cancer 67:626-663). (Carbon and carbohydrate metabolism is reviewed in Stryer, L. (1995) Biochemistry W. H. Freeman and Company, New York N.Y.; Lehninger, A. L. (1982) Principles of Biochemistry Worth Publishers Inc., New York N.Y.; and Lodish, H. et al. (1995) Molecular Cell Biology Scientific American Books, New York N.Y.)

Glycolysis

Enzymes of the glycolytic pathway convert the sugar glucose to pyruvate while simultaneously producing ATP. The pathway also provides building blocks for the synthesis of cellular components such as long-chain fatty acids. After glycolysis, pyrvuate is converted to acetyl-Coenzyme A, which, in aerobic organisms, enters the citric acid cycle. Glycolytic enzymes include hexokinase, phosphoglucose isomerase, phosphofructokinase, aldolase, triose phosphate isomerase, glyceraldehyde 3-phosphate dehydrogenase, phosphoglycerate kinase, phosphoglyceromutase, enolase, and pyruvate kinase. Of these, phosphofructokinase, hexokinase, and pyruvate kinase are important in regulating the rate of glycolysis.

Gluconeogenesis

Gluconeogenesis is the synthesis of glucose from noncarbohydrate precursors such as lactate and amino acids. The pathway, which functions mainly in times of starvation and intense exercise, occurs mostly in the liver and kidney. Responsible enzymes include pyruvate carboxylase, phosphoenolpyruvate carboxykinase, fructose 1,6-bisphosphatase, and glucose-6-phosphatase.

Pentose Phosohate Pathway

Pentose phosphate pathway enzymes are responsible for generating the reducing agent NADPH, while at the same time oxidizing glucose-6-phosphate to ribose-5-phosphate. Ribose-5-phosphate and its derivatives become part of important biological molecules such as ATP, Coenzyme A, NAD⁺, FAD, RNA, and DNA The pentose phosphate pathway has both oxidative and non-oxidative branches. The oxidative branch steps, which are catalyzed by the enzymes glucose-6-phosphate dehydrogenase, lactonase, and 6-phosphogluconate dehydrogenase, convert glucose-6-phosphate and NADP⁺ to ribulose-6-phosphate and NADPH. The non-oxidative branch steps, which are catalyzed by the enzymes phosphopentose isomerase, phosphopentose epimerase, transketolase, and transaldolase, allow the interconversion of three-, four-, five-, six-, and seven-carbon sugars.

Glucouronate Metabolism

Glucuronate is a monosaccharide which, in the form of D-glucuronic acid, is found in the GAGs chondroitin and dermatan. D-glucuronic acid is also important in the detoxification and excretion of foreign organic compounds such as phenol. Enzymes involved in glucuronate metabolism include UDP-glucose dehydrogenase and glucuronate reductase.

Disaccharide Metabolism

Disaccharides must be hydrolyzed to monosaccharides to be digested. Lactose, a disaccharide found in mil, is hydrolyzed to galactose and glucose by the enzyme lactase. Maltose is derived from plant starch and is hydrolyzed to glucose by the enzyme maltase. Sucrose is derived from plants and is hydrolyzed to glucose and fructose by the enzyme sucrase. Trehalose, a disaccharide found mainly in insects and mushrooms, is hydrolyzed to glucose by the enzyme trehalase (OMIM*275360 Trehalase; Ruf, J. et al. (1990) J. Biol. Chem. 265:1503415039). Lactase, maltase, sucrase, and trehalase are bound to mucosal cells lining the small intestine, where they participate in the digestion of dietary disaccharides. The enzyme lactose synthetase, composed of the catalytic subunit galactosyltransferase and the modifier subunit α-lactalbumin, converts UDP-galactose and glucose to lactose in the mammary glands.

Glycogen, Starch, and Chitin Metabolism

Glycogen is the storage form of carbohydrates in mammals. Mobilization of glycogen maintains glucose levels between meals and during muscular activity. Glycogen is stored mainly in the liver and in skeletal muscle in the form of cytoplasmic granules. These granules contain enzymes that catalyze the synthesis and degradation of glycogen, as well as enzymes that regulate these processes. Enzymes that catalyze the degradation of glycogen include glycogen phosphorylase, a transferase, α-1,6-glucosidase, and phosphoglucomutase. Enzymes that catalyze the synthesis of glycogen include UDP-glucose pyrophosphorylase, glycogen synthetase, a branching enzyme, and nucleoside diphosphokinase. The enzymes of glycogen synthesis and degradation are tightly regulated by the hormones insulin, glucagon, and epinephrine. Starch, a plant-derived polysaccharide, is hydrolyzed to maltose, maltotriose, and α-dextrin by α-amylase, an enzyme secreted by the salivary glands and pancreas. Chitin is a polysaccharide found in insects and crustacea. A chitotriosidase is secreted by macrophages and may play a role in the degradation of chitin-containing pathogens (Boot, R. G. et al. (1995) J. Biol. Chem. 270:26252-26256).

Peptidoglycans and Glycosaminoglycans

Glycosaminoglycans (GAGs) are anionic linear unbranched polysaccharides composed of repetitive disaccharide units. These repetitive units contain a derivative of an amino sugar, either glucosamine or galactosamine. GAGs exist free or as part of proteoglycans, large molecules composed of a core protein attached to one or more GAGs. GAGs are found on the cell surface, inside cells, and in the extracellular matrix. Changes in GAG levels are associated with several autoimmune diseases including autoimmune thyroid disease, autoimmune diabetes mellitus, and systemic lupus erythematosus (Hansen, C. et al. (1996) Clin. Exp. Rheum 14 (Suppl. 15):S59-S67). GAGs include chondroitin sulfate, keratan sulfate, heparin, heparan sulfate, dermatan sulfate, and hyaluronan

The GAG hyaluronan (HA) is found in the extracellular matrix of many cells, especially in soft connective tissues, and is abundant in synovial fluid (Pitsillides, A. A. et al. (1993) Int. J. Exp. Pathol. 74:27-34). HA seems to play important roles in cell regulation, development, and differentiation (Laurent, T. C. and J. R. Fraser (1992) FASEB J. 6:2397-2404). Hyaluronidase is an enzyme that degrades HA to oligosaccharides. Hyaluronidases may function in cell adhesion, infection, angiogenesis, signal transduction, reproduction, cancer, and inflammation.

Proteoglycans, also known as peptidoglycans, are found in the extracellular matrix of connective tissues such as cartilage and are essential for distributing the load in weight-bearing joints. Cell-surface-attached proteoglycans anchor cells to the extracellular matrix. Both extracellular and cell-surface proteoglycans bind growth factors, facilitating their binding to cell-surface receptors and subsequent triggering of signal transduction pathways.

Amino Acid and Nitrogen Metabolism

NH₄⁺ is assimilated into amino acids by the actions of two enzymes, glutamate dehydrogenase and glutamine synthetase. The carbon skeletons of amino acids come from the intermediates of glycolysis, the pentose phosphate pathway, or the citric acid cycle. Of the twenty amino acids used in proteins, humans can synthesize only thirteen (nonessential amino acids). The remaining nine must come from the diet (essential amino acids). Enzymes involved in nonessential amino acid biosynthesis include glutamate kinase dehydrogenase, pyrroline carboxylate reductase, asparagine synthetase, phenylalanine oxygenase, methionine adenosyltransferase, adenosylhomocysteinase, cystathionine β-synthase, cystathionine γ-lyase, phosphoglycerate dehydrogenase, phosphoserine transaminase, phosphoserine phosphatase, serine hydroxylmethyltransferase, and glycine synthase.

Metabolism of amino acids takes place almost entirely in the liver, where the amino group is removed by aminotransferases (transaminases), for example, alanine aminotransferase. The amino group is transferred to α-ketoglutarate to form glutamate. Glutamate dehydrogenase converts glutamate to NH₄⁺ and α-ketoglutarate. NH₄⁺ is converted to urea by the urea cycle which is catalyzed by the enzymes arginase, ornithine transcarbamoylase, arginosuccinate synthetase, and arginosuccinase. Carbamoyl phosphate synthetase is also involved in urea formation. Enzymes involved in the metabolism of the carbon skeleton of amino acids include serine dehydratase, asparaginase, glutaminase, propionyl CoA carboxylase, methylmalonyl CoA mutase, branched-chain α-keto dehydrogenase complex, isovaleryl CoA dehydrogenase, β-methylcrotonyl CoA carboxylase, phenylalanine hydroxylase, p-hydroxylphenylpyruvate hydroxylase, and homogentisate oxidase.

Polyamines, which include spermidine, putrescine, and spermine, bind tightly to nucleic acids and are abundant in rapidly proliferating cells. Enzymes involved in polyamine synthesis include ornithine decarboxylase.

Diseases involved in amino acid and nitrogen metabolism include hyperammonemia, carbamoyl phosphate synthetase deficiency, urea cycle enzyme deficiencies, methylmalonic aciduria, maple syrup disease, alcaptonuria, and phenylketonuria.

Energy Metabolism

Cells derive energy from metabolism of ingested compounds that may be roughly categorized as carbohydrates, fats, or proteins. Energy is also stored in polymers such as triglycerides (fats) and glycogen (carbohydrates). Metabolism proceeds along separate reaction pathways connected by key intermediates such as acetyl coenzyme A (acetyl-CoA). Metabolic pathways feature anaerobic and aerobic degradation, coupled with the energy-requiring reactions such as phosphorylation of adenosine diphosphate (ADP) to the triphosphate (ATP) or analogous phosphorylations of guanosine (GDP/GTP), uridine (UDP/UTP), or cytidine (CDP/CTP). Subsequent dephosphorylation of the triphosphate drives reactions needed for cell maintenance, growth, and proliferation.

Digestive enzymes convert carbohydrates and sugars to glucose; fructose and galactose are converted in the liver to glucose. Enzymes involved in these conversions include galactose-1-phosphate uridyl transferase and UDP-galactose-4 epimerase. In the cytoplasm, glycolysis converts glucose to pyruvate in a series of reactions coupled to ATP synthesis.

Pyruvate is transported into the mitochondria and converted to acetyl-CoA for oxidation via the citric acid cycle, involving pyruvate dehydrogenase components, dihydrolipoyl transacetylase, and dihydrolipoyl dehydrogenase. Enzymes involved in the citric acid cycle include: citrate synthetase, aconitases, isocitrate dehydrogenase, alpha-ketoglutarate dehydrogenase complex including transsuccinylases, succinyl CoA synthetase, succinate dehydrogenase, fumarases, and malate dehydrogenase. Acetyl CoA is oxidized to CO₂ with concomitant formation of NADH, FADH₂, and GTP. In oxidative phosphorylation, the transport of electrons from NADH and FADH₂ to oxygen by dehydrogenases is coupled to the synthesis of ATP from ADP and Pᵢ by the F₀F₁ ATPase complex in the mitochondrial inner membrane. Enzyme complexes responsible for electron transport and ATP synthesis include the F₀F₁ ATPase complex, ubiquinone(CoQ)-cytochrome c reductase, ubiquinone reductase, cytochrome b, cytochrome c₁, FeS protein, and cytochrome c oxidase.

Triglycerides are hydrolyzed to fatty acids and glycerol by lipases. Glycerol is then is phosphorylated to glycerol-3-phosphate by glycerol kinase and glycerol phosphate dehydrogenase, and degraded by the glycolysis. Fatty acids are transported into the mitochondria as fatty acyl-carnitine esters and undergo oxidative degradation.

In addition to metabolic disorders such as diabetes and obesity, disorders of energy metabolism are associated with cancers (Dorward, A. et al. (1997) J. Bioenerg. Biomembr. 29:385-392), autism (Lombard, J. (1998) Med. Hypotheses 50:497-500), neurodegenerative disorders (Alexi, T. et al. (1998) Neuroreport 9:R57-64), and neuromuscular disorders (DiMauro, S. et al. (1998) Biochim. Biophys. Acta 1366:199-210). The myocardium is heavily dependent on oxidative metabolism, so metabolic dysfunction often leads to heart disease (DiMauro, S. and M. Hirano (1998) Curr. Opin. Cardiol. 13:190-197).

For a review of energy metabolism enzymes and intermediates, see Stryer, L. et al. (1995) Biochemistry, W. H. Freeman and Co., San Francisco Calif., pp.443-652. For a review of energy metabolism regulation, see Lodish, H. et al. (1995) Molecular Cell Biology, Scientific American Books, New York N.Y., pp. 744-770.

Cofactor Metabolism

Cofactors, including coenzymes and prosthetic groups, are small molecular weight inorganic or organic compounds that are required for the action of an enzyme. Many cofactors contain vitamins as a component. Cofactors include thiamine pyrophosphate, flavin adenine dinucleotide, flavin mononucleotide, nicotinamide adenine dinucleotide, pyridoxal phosphate, coenzyme A, tetrahydrofolate, lipoamide, and heme. The vitamins biotin and cobalamin are associated with enzymes as well. Heme, a prosthetic group found in myoglobin and hemoglobin, consists of protoporphyrin group bound to iron. Porphyrin groups contain four substituted pyrroles covalently joined in a ring, often with a bound metal atom. Enzymes involved in porphyrin synthesis include δ-aminolevulinate synthase, δ-aminolevulinate dehydrase, porphobilinogen deaminase, and cosynthase. Deficiencies in heme formation cause porphyrias. Heme is broken down as a part of erythrocyte turnover. Enzymes involved in heme degradation include heme oxygenase and biliverdin reductase.

Iron is a required cofactor for many enzymes. Besides the heme-containing enzymes, iron is found in iron-sulfur clusters in proteins including aconitase, succinate dehydrogenase, and NADH-Q reductase. Iron is transported in the blood by the protein transferrin Binding of transferrin to the transferrin receptor on cell surfaces allows uptake by receptor mediated endocytosis. Cytosolic iron is bound to ferritin protein.

A molybdenum-containing cofactor (molybdopterin) is found in enzymes including sulfite oxidase, xanthine dehydrogenase, and aldehyde oxidase. Molybdopterin biosynthesis is performed by two molybdenum cofactor synthesizing enzymes. Deficiencies in these enzymes cause mental retardation and lens dislocation. Other diseases caused by defects in cofactor metabolism include pernicious anemia and methylmalonic aciduria.

Secretion and Trafficking

Eukaryotic cells are bound by a lipid bilayer membrane and subdivided into functionally distinct, membrane bound compartments. The membranes maintain the essential differences between the cytosol, the extracelluar environment, and the lumenal space of each intracellular organelle. As lipid membranes are highly impermeable to most polar molecules, transport of essential nutrients, metabolic waste products, cell signaling molecules, macromolecules and proteins across lipid membranes and between organelles must be mediated by a variety of transport-associated molecules.

Protein Trafficking

In eukaryotes, some proteins are synthesized on ER-bound ribosomes, co-translationally imported into the ER, delivered from the ER to the Golgi complex for post-translational processing and sorting, and transported from the Golgi to specific intracellular and extracellular destinations. All cells possess a constitutive transport process which maintains homeostasis between the cell and its environment. In many differentiated cell types, the basic machinery is modified to carry out specific transport functions. For example, in endocrine glands, hormones and other secreted proteins are packaged into secretory granules for regulated exocytosis to the cell exterior. In macrophage, foreign extracellular material is engulfed (phagocytosis) and delivered to lysosomes for degradation. In fat and muscle cells, glucose transporters are stored in vesicles which fuse with the plasma membrane only in response to insulin stimulation.

The Secretory Pathway

Synthesis of most integral membrane proteins, secreted proteins, and proteins destined for the lumen of a particular organelle occurs on ER-bound ribosomes. These proteins are co-translationally imported into the ER. The proteins leave the ER via membrane-bound vesicles which bud off the ER at specific sites and fuse with each other (homotypic fusion) to form the ER-Golgi Intermediate Compartment (ERGIC). The ERGIC matures progressively through the cis, medial, and trans cisternal stacks of the Golgi, modifying the enzyme composition by retrograde transport of specific Golgi enzymes. In this way, proteins moving through the Golgi undergo post-translational modification, such as glycosylation. The final Golgi compartment is the Trans-Golgi Network (TGN), where both membrane and lumenal proteins are sorted for their final destination. Transport vesicles destined for intracellular compartments, such as the lysosome, bud off the TGN. What remains is a secretory vesicle which contains proteins destined for the plasma membrane, such as receptors, adhesion molecules, and ion channels, and secretory proteins, such as hormones, neurotransmitters, and digestive enzymes. Secretory vesicles eventually fuse with the plasma membrane (Glick, B. S. and V. Malhotra (1998) Cell 95:883-889).

The secretory process can be constitutive or regulated. Most cells have a constitutive pathway for secretion, whereby vesicles derived from maturation of the TGN require no specific signal to fuse with the plasma membrane. In many cells, such as endocrine cells, digestive cells, and neurons, vesicle pools derived from the TGN collect in the cytoplasm and do not fuse with the plasma membrane until they are directed to'by a specific signal.

Endocytosis

Endocytosis, wherein cells internalize material from the extracellular environment, is essential for transmission of neuronal, metabolic, and proliferative signals; uptake of many essential nutrients; and defense against invading organisms. Most cells exhibit two forms of endocytosis. The first, phagocytosis, is an actin-driven process exemplified in macrophage and neutrophils. Material to be endocytosed contacts numerous cell surface receptors which stimulate the plasma membrane to extend and surround the particle, enclosing it in a membrane-bound phagosome. In the mammalian immune system, IgG-coated particles bind Fc receptors on the surface of phagocytic leukocytes. Activation of the Fc receptors initiates a signal cascade involving src-family cytosolic kinases and the monomeric GTP-binding (G) protein Rho. The resulting actin reorganization leads to phagocytosis of the particle. This process is an important component of the humoral immune response, allowing the processing and presentation of bacterial-derived peptides to antigen-specific T-lymphocytes.

The second form of endocytosis, pinocytosis, is a more generalized uptake of material from the external milieu. Like phagocytosis, pinocytosis is activated by ligand binding to cell surface receptors. Activation of individual receptors stimulates an internal response that includes coalescence of the receptor-ligand complexes and formation of clathrin-coated pits. Invagination of the plasma membrane at clathrin-coated pits produces an endocytic vesicle within the cell cytoplasm. These vesicles undergo homotypic fusion to form an early endosomal (EE) compartment. The tubulovesicular EE serves as a sorting site for incoming material. ATP-driven proton pumps in the EE membrane lowers the pH of the EE lumen (pH 6.3-6.8). The acidic environment causes many ligands to dissociate from their receptors. The receptors, along with membrane and other integral membrane proteins, are recycled back to the plasma membrane by budding off the tubular extensions of the EE in recycling vesicles (RV). This selective removal of recycled components produces a carrier vesicle containing ligand and other material from the external environment. The carrier vesicle fuses with TGN-derived vesicles which contain hydrolytic enzymes. The acidic environment of the resulting late endosome (LE) activates the hydrolytic enzymes which degrade the ligands and other material. As digestion takes place, the LE fuses with the lysosome where digestion is completed (Mellman, I. (1996) Annu. Rev. Cell Dev. Biol. 12:575-625).

Recycling vesicles may return directly to the plasma membrane. Receptors internalized and returned directly to the plasma membrane have a turnover rate of 2-3 minutes. Some RVs undergo microtubule-directed relocation to a perinuclear site, from which they then return to the plasma membrane. Receptors following this route have a turnover rate of 5-10 minutes. Still other RVs are retained within the cell until an appropriate signal is received (Mellman, supra; and James, D. E. et al. (1994) Trends Cell Biol. 4:120-126).

Vesicle Formation

Several steps in the transit of material along the secretory and endocytic pathways require the formation of transport vesicles. Specifically, vesicles form at the transitional endoplasmic reticulum (tER), the rim of Golgi cisternae, the face of the Trans-Golgi Network (TGN), the plasma membrane (PM), and tubular extensions of the endosomes. The process begins with the budding of a vesicle out of the donor membrane. The membrane-bound vesicle contains proteins to be transported and is surrounded by a protective coat made up of protein subunits recruited from the cytosol. The initial budding and coating processes are controlled by a cytosolic ras-like GTP-binding protein, ADP-ribosylating factor (Arf), and adapter proteins (AP). Different isoforms of both Arf and AP are involved at different sites of budding. Another small G-protein, dynamin, forms a ring complex around the neck of the forming vesicle and may provide the mechanochemical force to accomplish the final step of the budding process. The coated vesicle complex is then transported through the cytosol. During the transport process, Arf-bound GTP is hydrolyzed to GDP and the coat dissociates from the transport vesicle (West, M. A. et al. (1997) J. Cell Biol. 138:1239-1254). Two different classes of coat protein have also been identified Clathrin coats form on the TGN and PM surfaces, whereas coatomer or COP coats form on the ER and Golgi. COP coats can further be distinguished as COPI, involved in retrograde traffic through the Golgi and from the Golgi to the ER, and COPII, involved in anterograde traffic from the ER to the Golgi (Mellman, supra). The COP coat consists of two major components, a G-protein (Arf or Sar) and coat protomer (coatomer). Coatomer is an equimolar complex of seven proteins, termed alpha-, beta-, beta'-, gamma-, delta-, epsilon- and zeta-COP. (Harter, C. and F. T. Wieland (1998) Proc. Natl. Acad. Sci. USA 95:11649-11654.)

Membrane Fusion

Transport vesicles undergo homotypic or heterotypic fusion in the secretory and endocytotic pathways. Molecules required for appropriate targeting and fusion of vesicles with their target membrane include proteins incorporated in the vesicle membrane, the target membrane, and proteins recruited from the cytosol. During budding of the vesicle from the donor compartment, an integral membrane protein, VAMP (vesicle-associated membrane protein) is incorporated into the vesicle. Soon after the vesicle uncoats, a cytosolic prenylated GTP-binding protein, Rab (a member of the Ras superfamily), is inserted into the vesicle membrane. GTP-bound Rab proteins are directed into nascent transport vesicles where they interact with VAMP. Following vesicle transport, GTPase activating proteins (GAPs) in the target membrane convert Rab proteins to the GDP-bound form A cytosolic protein, guanine-nucleotide dissociation inhibitor (GDI) helps return GDP-bound Rab proteins to their membrane of origin. Several Rab isoforms have been identified and appear to associate with specific compartments within the cell. Rab proteins appear to play a role in mediating the function of a viral gene, Rev, which is essential for replication of HIV-1, the virus responsible for AIDS (Flavell, R. A. et al. (1996) Proc. Natl. Acad. Sci. USA 93:4421-4424).

Docking of the transport vesicle with the target membrane involves the formation of a complex between the vesicle SNAP receptor (v-SNARE), target membrane (t-) SNAREs, and certain other membrane and cytosolic proteins. Many of these other proteins have been identified although their exact functions in the docking complex remain uncertain (Tellam, J. T. et al. (1995) J. Biol. Chem. 270:5857-5863; and Hata, Y. and T. C. Sudhof (1995) J. Biol. Chem. 270:13022-13028). N-ethylmaleimide sensitive factor (NSF) and soluble NSF-attachment protein (α-SNAP and, β-SNAP) are two such proteins that are conserved from yeast to man and function in most intracellular membrane fusion reactions. Sec1 represents a family of yeast proteins that function at many different stages in the secretory pathway including membrane fusion Recently, mammalian homologs of Sec1, called Munc-18 proteins, have been identified (Katagiri, H. et al. (1995) J. Biol. Chem 270:4963-4966; Hata et al. supra).

The SNARE complex involves three SNARE molecules, one in the vesicular membrane and two in the target membrane. Synaptotagmin is an integral membrane protein in the synaptic vesicle which associates with the t-SNARE syntaxin in the docking complex. Synaptotagmin binds calcium in a complex with negatively charged phospholipids, which allows the cytosolic SNAP protein to displace synaptotagmin from syntaxin and fusion to occur. Thus, synaptotagmin is a negative regulator of fusion in the neuron (Littleton, J. T. et al. (1993) Cell 74:1125-1134). The most abundant membrane protein of synaptic vesicles appears to be the glycoprotein synaptophysin, a 38 kDa protein with four transmembrane domains.

Specificity between a vesicle and its target is derived from the v-SNARE, t-SNAREs, and associated proteins involved. Different isoforms of SNAREs and Rabs show distinct cellular and subcellular distributions. VAMP-1/synaptobrevin, membrane-anchored synaptosome-associated protein of 25 kDa (SNAP-25), syntaxin-1, Rab3A, Rab15, and Rab23 are predominantly expressed in the brain and nervous system. Different syntaxin, VAMP, and Rab proteins are associated with distinct subcellular compartments and their vesicular carriers.

Nuclear Transport

Transport of proteins and RNA between the nucleus and the cytoplasm occurs through nuclear pore complexes (NPCs). NPC-mediated transport occurs in both directions through the nuclear envelope. All nuclear proteins are imported from the cytoplasm, their site of synthesis. tRNA and mRNA are exported from the nucleus, their site of synthesis, to the cytoplasm, their site of function. Processing of small nuclear RNAs involves export into the cytoplasm, assembly with proteins and modifications such as hypermethylation to produce small nuclear ribonuclear proteins (s RNs), and subsequent import of the snRNPs back into the nucleus. The assembly of ribosomes requires the initial import of ribosomal proteins from the cytoplasm, their incorporation with RNA into ribosomal subunits, and export back to the cytoplasm. (Görlich, D. and I. W. Mattaj (1996) Science 271:1513-1518.)

The transport of proteins and mRNAs across the NPC is selective, dependent on nuclear localization signals, and generally requires association with nuclear transport factors. Nuclear localization signals (NLS) consist of short stretches of amino acids enriched in basic residues. NLS are found on proteins that are targeted to the nucleus, such as the glucocorticoid receptor. The NLS is recognized by the NLS receptor, importin, which then interacts with the monomeric GTP-binding protein Ran. This NLS protein/receptor/Ran complex navigates the nuclear pore with the help of the homodimeric protein nuclear transport factor 2 (NTF2). NTF2 binds the GDP-bound form of Ran and to multiple proteins of the nuclear pore complex containing FXFG repeat motifs, such as p62. (Paschal, B. et al. (1997) J. Biol. Chem. 272:21534-21539; and Wong, D. H. et al. (1997) Mol. Cell Biol. 17:3755-3767). Some proteins are dissociated before nuclear mRNAs are transported across the NPC while others are dissociated shortly after nuclear mRNA transport across the NPC and are reimported into the nucleus.

Disease Correlation

The etiology of numerous human diseases and disorders can be attributed to defects in the transport or secretion of proteins. For example, abnormal hormonal secretion is linked to disorders such as diabetes insipidus (vasopressin), hyper- and hypoglycemia (insulin, glucagon), Grave's disease and goiter (thyroid hormone), and Cushing's and Addison's diseases (adrenocorticotropic hormone, ACTH). Moreover, cancer cells secrete excessive amounts of hormones or other biologically active peptides. Disorders related to excessive secretion of biologically active peptides by tumor cells include fasting hypoglycemia due to increased insulin secretion from insulinoma-islet cell tumors; hypertension due to increased epinephrine and norepinephrine secreted from pheochromocytomas of the adrenal medulla and sympathetic paraganglia; and carcinoid syndrome, which is characterized by abdominal cramps, diarrhea, and valvular heart disease caused by excessive amounts of vasoactive substances such as serotonin, bradykinin, histamine, prostaglandins, and polypeptide hormones, secreted from intestinal tumors. Biologically active peptides that are ectopically synthesized in and secreted from tumor cells include ACTH and vasopressin (lung and pancreatic cancers); parathyroid hormone (lung and bladder cancers); calcitonin (lung and breast cancers); and thyroid-stimulating hormone (medullary thyroid carcinoma). Such peptides may be useful as diagnostic markers for tumorigenesis(Schwartz, M. Z. (1997) Semin. Pediatr. Surg. 3:141-146; and Said, S. I. and G. R. Faloona (1975) N. Engl. J. Med. 293:155-160).

Defective nuclear transport may play a role in cancer. The BRCA1 protein contains three potential NLSs which interact with importin alpha, and is transported into the nucleus by the importin/NPC pathway. In breast cancer cells the BRCA1 protein is aberrantly localized in the cytoplasm. The mislocation of the BRCA1 protein in breast cancer cells may be due to a defect in the NPC nuclear import pathway (Chen, C. F. et al. (1996) J. Biol. Chem. 271:32863-32868).

It has been suggested that in some breast cancers, the tumor-suppressing activity of p53 is inactivated by the sequestration of the protein in the cytoplasm, away from its site of action in the cell nucleus. Cytoplasmic wild-type p53 was also found inhuman cervical carcinoma cell lines. (Moll, U. M. et al. (1992) Proc. Natl. Acad. Sci. USA 89:7262-7266; and Liang, X. H. et al. (1993) Oncogene 8:2645-2652.)

Environmental Responses

Organisms respond to the environment by a number of pathways. Heat shock proteins, including hsp 70, hsp60, hsp90, and hsp 40, assist organisms in coping with heat damage to cellular proteins.

Aquaporins (AQP) are channels that transport water and, in some cases, nonionic small solutes such as urea and glycerol. Water movement is important for a number of physiological processes including renal fluid filtration, aqueous humor generation in the eye, cerebrospinal fluid production in the brain, and appropriate hydration of the lung. Aquaporins are members of the major intrinsic protein (MIP) family of membrane transporters (King, L. S. and P. Agre (1996) Annu. Rev. Physiol. 58:619-648; Ishibashi, K. et al. (1997) J. Biol. Chem. 272:20782-20786). The study of aquaporins may have relevance to understanding edema formation and fluid balance in both normal physiology and disease states (King, supra). Mutations in AQP2 cause autosomal recessive nephrogenic diabetes insipidus (OMIM*107777 Aquaporin 2; AQP2). Reduced AQP4 expression in skletal muscle may be associated with Duchenne muscular dystrophy (Frigeri, A. et al. (1998) J. Clin. Invest. 102:695-703). Mutations in AQPO cause autosomal dominant cataracts in the mouse (OMIM *154050 Major Intrinsic Protein of Lens Fiber; MIP).

The metallothioneins (M Is) are a group of small (61 amino acids), cysteine-rich proteins that bind heavy metals such as cadmium, zinc, mercury, lead, and copper and are thought to play a role in metal detoxification or the metabolism and homeostasis of metals. Arsenite-resistance proteins have been identified in hamsters that are resistant to toxic levels of arsenite (Rossman, T. G. et al. (1997) Mutat. Res. 386:307-314).

Humans respond to light and odors by specific protein pathways. Proteins involved in light perception include rhodopsin, transducin, and cGMP phosphodiesterase. Proteins involved in odor perception include multiple olfactory receptors. Other proteins are important inhuman Circadian rhythms and responses to wounds.

Immunity and Host Defense

All vertebrates have developed sophisticated and complex immune systems that provide protection from viral, bacterial, fungal and parasitic infections. Included in these systems are the processes of humoral immunity, the complement cascade and the inflammatory response (Paul, W. E. (1993) Fundamental Immunology, Raven Press, Ltd., New York N.Y., pp.1-20).

The cellular components of the humoral immune system include six different types of leukocytes: monocytes, lymphocytes, polymorphonuclear granulocytes (consisting of neutrophils, eosinophils, and basophils) and plasma cells. Additionally, fragments of megakaryocytes, a seventh type of white blood cell in the bone marrow, occur in large numbers in the blood as platelets.

Leukocytes are formed from two stem cell lineages in bone marrow. The myeloid stem cell line produces granulocytes and monocytes and, the lymphoid stem cell produces lymphocytes. Lymphoid cells travel to the thymus, spleen and lymph nodes, where they mature and differentiate into lymphocytes. Leukocytes are responsible for defending the body against invading pathogens.

Neutrophils and monocytes attack invading bacteria, viruses, and other pathogens and destroy them by phagocytosis. Monocytes enter tissues and differentiate into macrophages which are extremely phagocytic. Lymphocytes and plasma cells are a part of the immune system which recognizes specific foreign molecules and organisms and inactivates them, as well as signals other cells to attack the invaders.

Granulocytes and monocytes are formed and stored in the bone marrow until needed. Megakaryocytes are produced in bone marrow, where they fragment into platelets and are released into the bloodstream. The main function of platelets is to activate the blood clotting mechanism. Lymphocytes and plasma cells are produced in various lymphogenous organs, including the lymph nodes, spleen, thymus, and tonsils.

Both neutrophils and macrophages exhibit chemotaxis towards sites of inflammation. Tissue inflammation in response to pathogen invasion results in production of chemo-attractants for leukocytes, such as endotoxins or other bacterial products, prostaglandins, and products of leukocytes or platelets.

Basophils participate in the release of the chemicals involved in the inflammatory process. The main function of basophils is secretion of these chemicals to such a degree that they have been referred to as “unicellular endocrine glands.” A distinct aspect of basophilic secretion is that the contents of granules go directly into the extracellular environment, not into vacuoles as occurs with neutrophils, eosinophils and monocytes. Basophils have receptors for the Fc fragment of immunoglobulin E (IgE) that are not present on other leukocytes. Crosslinking of membrane IgE with anti-IgE or other ligands triggers degranulation.

Eosinophils are bi- or multi-nucleated white blood cells which contain eosinophilic granules. Their plasma membrane is characterized by Ig receptors, particularly IgG and IgE. Generally, eosinophils are stored in the bone marrow until recruited for use at a site of inflammation or invasion. They have specific functions in parasitic infections and allergic reactions, and are thought to detoxify some of the substances released by mast cells and basophils which cause inflammation. Additionally, they phagocytize antigen-antibody complexes and further help prevent spread of the inflammation.

Macrophages are monocytes that have left the blood stream to settle in tissue. Once monocytes have migrated into tissues, they do not reenter the bloodstream. The mononuclear phagocyte system is comprised of precursor cells in the bone marrow, monocytes in circulation, and macrophages in tissues. The system is capable of very fast and extensive phagocytosis. A macrophage may phagocytize over 100 bacteria, digest them and extrude residues, and then survive for many more months. Macrophages are also capable of ingesting large particles, including red blood cells and malarial parasites. They increase several-fold in size and transform into macrophages that are characteristic of the tissue they have entered, surviving in tissues for several months.

Mononuclear phagocytes are essential in defending the body against invasion by foreign pathogens, particularly intracellular microorganisms such as M. tuberculosis, listeria, leishmania and toxoplasma. Macrophages can also control the growth of tumorous cells, via both phagocytosis and secretion of hydrolytic enzymes. Another important function of macrophages is that of processing antigen and presenting them in a biochemically modified form to lymphocytes.

The immune system responds to invading microorganisms in two major ways: antibody production and cell mediated responses. Antibodies are immunoglobulin proteins produced by B-lymphocytes which bind to specific antigens and cause inactivation or promote destruction of the antigen by other cells. Cell-mediated immune responses involve T-lymphocytes (T cells) that react with foreign antigen on the surface of infected host cells. Depending on the type of T cell, the infected cell is either killed or signals are secreted which activate macrophages and other cells to destroy the infected cell (Paul, supra).

T-lymphocytes originate in the bone marrow or liver in fetuses. Precursor cells migrate via the blood to the thymus, where they are processed to mature into T-lymphocytes. This processing is crucial because of positive and negative selection of T cells that will react with foreign antigen and not with self molecules. After processing, T cells continuously circulate in the blood and secondary lymphoid tissues, such as lymph nodes, spleen, certain epithelium-associated tissues in the gastrointestinal tract, respiratory tract and skin. When T-lymphocytes are presented with the complementary antigen, they are stimulated to proliferate and release large numbers of activated T cells into the lymph system and the blood system. These activated T cells can survive and circulate for several days. At the same time, T memory cells are created, which remain in the lymphoid tissue for months or years. Upon subsequent exposure to that specific antigen, these memory cells will respond more rapidly and with a stronger response than induced by the original antigen. This creates an “immunological memory” that can provide immunity for years.

There are two major types of T cells: cytotoxic T cells destroy infected host cells, and helper T cells activate other white blood cells via chemical signals. One class of helper cell, T_{H}1, activates macrophages to destroy ingested microorganisms, while another, T_{H}2, stimulates the production of antibodies by B cells.

Cytotoxic T cells directly attack the infected target cell. In virus-infected cells, peptides derived from viral proteins are generated by the proteasome. These peptides are transported into the ER by the transporter associated with antigen processing (TAP) (Pamer, E. and P. Cresswell (1998) Annu. Rev. Immunol. 16:323-358). Once inside the ER, the peptides bind MHC I chains, and the peptide/MHC I complex is transported to the cell surface. Receptors on the surface of T cells bind to antigen presented on cell surface MHC molecules. Once activated by binding to antigen, T cells secrete γ-interferon, a signal molecule that induces the expression of genes necessary for presenting viral (or other) antigens to cytotoxic T cells. Cytotoxic T cells kill the infected cell by stimulating programmed cell death.

Helper T cells constitute up to 75% of the total T cell population. They regulate the immune functions by producing a variety of lymphokines that act on other cells in the immune system and on bone marrow. Among these lymphokines are: interleukins-2,3,4,5,6; granulocyte-monocyte colony stimulating factor, and γ-interferon.

Helper T cells are required for most B cells to respond to antigen. When an activated helper cell contacts a B cell its centrosome and Golgi apparatus become oriented toward the B cell, aiding the directing of signal molecules, such as transmembrane-bound protein called CD40 ligand, onto the B cell surface to interact with the CD40 transmembrane protein Secreted signals also help B cells to proliferate and mature and, in some cases, to switch the class of antibody being produced.

B-lymphocytes (B cells) produce antibodies which react with specific antigenic proteins presented by pathogens. Once activated, B cells become filled with extensive rough endoplasmic reticulum and are known as plasma cells. As with T cells, interaction of B cells with antigen stimulates proliferation of only those B cells which produce antibody specific to that antigen. There are five classes of antibodies, known as immunoglobulins, which together comprise about 20% of total plasma protein. Each class mediates a characteristic biological response after antigen binding. Upon activation by specific antigen B cells switch from making membrane-bound antibody to secretion of that antibody.

Antibodies, or immunoglobulins (g), are the founding members of the Ig superfamily and the central components of the humoral immune response. Antibodies are either expressed on the surface of B cells or secreted by B cells into the circulation. Antibodies bind and neutralize blood-borne foreign antigens. The prototypical antibody is a tetramer consisting of two identical heavy polypeptide chains (H-chains) and two identical light polypeptide chains (L-chains) interlinked by disulfide bonds. This arrangement confers the characteristic Y-shape to antibody molecules. Antibodies are classified based on their H-chain composition. The five antibody classes, IgA, IgD, IgE, IgG and IgM, are defined by the α, δ, ε, γ, and μ H-chain types. There are two types of L-chains, κ and λ, either of which may associate as a pair with any H-chain pair. IgG, the most common class of antibody found in the circulation, is tetrameric, while the other classes of antibodies are generally variants or multimers of this basic structure.

H-chains and L-chains each contain an N-terminal variable region and a C-terminal constant region. Both H-chains and L-chains contain repeated Ig domains. For example, a typical H-chain contains four Ig domains, three of which occur within the constant region and one of which occurs within the variable region and contributes to the formation of the antigen recognition site. Likewise, a typical L-chain contains two Ig domains, one of which occurs within the constant region and one of which occurs within the variable region. In addition, H chains such as μ have been shown to associate with other polypeptides during differentiation of the B cell.

Antibodies can be described in terms of their two main functional domains. Antigen recognition is mediated by the Fab (antigen binding fragment) region of the antibody, while effector functions are mediated by the Fc (crystallizable fragment) region. Binding of antibody to an antigen, such as a bacterium, triggers the destruction of the antigen by phagocytic white blood cells such as macrophages and neutrophils. These cells express surface receptors that specifically bind to the antibody Fc region and allow the phagocytic cells to engulf, ingest, and degrade the antibody-bound antigen. The Fc receptors expressed by phagocytic cells are single-pass transmembrane glycoproteins of about 300 to 400 amino acids (Sears, D. W. et al. (1990) J. Immunol. 144:371-378). The extracellular portion of the Fc receptor typically contains two or three Ig domains.

Diseases which cause over- or under-abundance of any one type of leukocyte usually result in the entire immune defense system becoming involved. A well-known autoimmune disease is AIDS (Acquired Immunodeficiency Syndrome) where the number of helper T cells is depleted, leaving the patient susceptible to infection by microorganisms and parasites. Another widespread medical condition attributable to the immune system is that of allergic reactions to certain antigens. Allergic reactions include: hay fever, asthma, anaphylaxis, and urticaria (hives). Leukemias are an excess production of white blood cells, to the point where a major portion of the body's metabolic resources are directed solely at proliferation of white blood cells, leaving other tissues to starve. Leukopenia or agranulocytosis occurs when the bone marrow stops producing white blood cells. This leaves the body unprotected against foreign microorganisms, including those which normally inhabit skin, mucous membranes, and gastrointestinal tract. If all white blood cell production stops completely, infection will occur within two days and death may follow only 1 to 4 days later.

Impaired phagocytosis occurs in several diseases, including monocytic leukemia, systemic lupus, and granulomatous disease. In such a situation, macrophages can phagocytize normally, but the enveloped organism is not killed. A defect in the plasma membrane enzyme which converts oxygen to lethally reactive forms results in abscess formation in liver, lungs, spleen, lymph nodes, and beneath the skin. Eosinophilia is an excess of eosinophils commonly observed in patients with allergies (hay fever, asthma), allergic reactions to drugs, rheumatoid arthritis, and cancers (Hodgkin's disease, lung, and liver cancer) (Isselbacher, K. J. et al. (1994) Harrison's Principles of Internal Medicine, McGraw-Hill, Inc., New York N.Y.).

Host defense is further augmented by the complement system. The complement system serves as an effector system and is involved in infectious agent recognition. It can function as an independent immune network or in conjunction with other humoral immune responses. The complement system is comprised of numerous plasma and membrane proteins that act in a cascade of reaction sequences whereby one component activates the next. The result is a rapid and amplified response to infection through either an inflammatory response or increased phagocytosis.

The complement system has more than 30 protein components which can be divided into functional groupings including modified serine proteases, membrane-binding proteins and regulators of complement activation. Activation occurs through two different pathways the classical and the alternative. Both pathways serve to destroy infectious agents through distinct triggering mechanisms that eventually merge with the involvement of the component C3.

The classical pathway requires antibody binding to infectious agent antigens. The antibodies serve to define the target and initiate the complement system cascade, culminating in the destruction of the infectious agent. In this pathway, since the antibody guides initiation of the process, the complement can be seen as an effector arm of the humoral immune system.

The alternative pathway of the complement system does not require the presence of pre-existing antibodies for targeting infectious agent destruction. Rather, this pathway, through low levels of an activated component, remains constantly primed and provides surveillance in the non-immune host to enable targeting and destruction of infectious agents. In this case foreign material triggers the cascade, thereby facilitating phagocytosis or lysis (Paul, supra, pp.918-919).

Another important component of host defense is the process of inflammation. Inflammatory responses are divided into four categories on the basis of pathology and include allergic inflammation, cytotoxic antibody mediated inflammation, immune complex mediated inflammation and monocyte mediated inflammation. Inflammation manifests as a combination of each of these forms with one predominating.

Allergic acute inflammation is observed in individuals wherein specific antigens stimulate IgE antibody production. Mast cells and basophils are subsequently activated by the attachment of antigen-IgE complexes, resulting in the release of cytoplasmic granule contents such as histamine. The products of activated mast cells can increase vascular permeability and constrict the smooth muscle of breathing passages, resulting in anaphylaxis or asthma. Acute inflammation is also mediated by cytotoxic antibodies and can result in the destruction of tissue through the binding of complement-fixing antibodies to cells. The responsible antibodies are of the IgG or IgM types. Resultant clinical disorders include autoimmune hemolytic anemia and thrombocytopenia as associated with systemic lupus erythematosis.

Immune complex mediated acute inflammation involves the IgG or IgM antibody types which combine with antigen to activate the complement cascade. When such immune complexes bind to neutrophils and macrophages they activate the respiratory burst to form protein- and vessel-damaging agents such as hydrogen peroxide, hydroxyl radical, hypochlorous acid, and chloramines. Clinical manifestations include rheumatoid arthritis and systemic lupus erythematosus.

In chronic inflammation or delayed-type hypersensitivity, macrophages are activated and process antigen for presentation to T cells that subsequently produce lymphokines and monokines. This type of inflammatory response is likely important for defense against intracellular parasites and certain viruses. Clinical associations include, granulomatous disease, tuberculosis, leprosy, and sarcoidosis (Paul, W. E., supra, pp.1017-1018).

Extracellular Information Transmission Molecules

Intercellular communication is essential for the growth and survival of multicellular organisms, and in particular, for the function of the endocrine, nervous, and immune systems. In addition, intercellular communication is critical for developmental processes such as tissue construction and organogenesis, in which cell proliferation, cell differentiation, and morphogenesis must be spatially and temporally regulated in a precise and coordinated manner. Cells communicate with one another through the secretion and uptake of diverse types of signaling molecules such as hormones, growth factors, neuropeptides, and cytokines.

Hormones

Hormones are signaling molecules that coordinately regulate basic physiological processes from embryogenesis throughout adulthood. These processes include metabolism, respiration, reproduction, excretion, fetal tissue differentiation and organogenesis, growth and development, homeostasis, and the stress response. Hormonal secretions and the nervous system are tightly integrated and interdependent. Hormones are secreted by endocrine glands, primarily the hypothalamus and pituitary, the thyroid and parathyroid, the pancreas, the adrenal glands, and the ovaries and testes.

The secretion of hormones into the circulation is tightly controlled Hormones are often secreted in diurnal, pulsatile, and cyclic patterns. Hormone secretion is regulated by perturbations in blood biochemistry, by other upstream-acting hormones, by neural impulses, and by negative feedback loops. Blood hormone concentrations are constantly monitored and adjusted to maintain opal, steady-state levels. Once secreted, hormones act only on those target cells that express specific receptors.

Most disorders of the endocrine system are caused by either hyposecretion or hypersecretion of hormones. Hyposecretion often occurs when a hormone's gland of origin is damaged or otherwise impaired. Hypersecretion often results from the proliferation of tumors derived from hormone-secreting cells. Inappropriate hormone levels may also be caused by defects in regulatory feedback loops or in the processing of hormone precursors. Endocrine malfunction may also occur when the target cell falls to respond to the hormone.

Hormones can be classified biochemically as polypeptides, steroids, eicosanoids, or amines. Polypeptides, which include diverse hormones such as insulin and growth hormone, vary in size and function and are often synthesized as inactive precursors that are processed intracellularly into mature, active forms. Amines, which include epinephrine and dopamine, are ammo acid derivatives that function in neuroendocrine signaling. Steroids, which include the cholesterol-derived hormones estrogen and testosterone, function in sexual development and reproduction. Eicosanoids, which include prostaglandins and prostacyclins, are fatty acid derivatives that function in a variety of processes. Most polypeptides and some amines are soluble in the circulation where they are highly susceptible to proteolytic degradation within seconds after their secretion. Steroids and lipids are insoluble and must be transported in the circulation by carrier proteins. The following discussion will focus primarily on polypeptide hormones.

Hormones secreted by the hypothalamus and pituitary gland play a critical role in endocrine function by coordinately regulating hormonal secretions from other endocrine glands in response to neural signals. Hypothalamic hormones include thyrotropin-releasing hormone, gonadotropin-releasing hormone, somatostatin, growth-hormone releasing factor, corticotropin-releasing hormone, substance P, dopamine, and prolactin-releasing hormone. These hormones directly regulate the secretion of hormones from the anterior lobe of the pituitary. Hormones secreted by the anterior pituitary include adrenocorticotropic hormone (ACTH), melanocyte-stimulating hormone, somatotropic hormones such as growth hormone and prolactin, glycoprotein hormones such as thyroid-stimulating hormone, luteinizing hormone (LH), and follicle-stimulating hormone (FSH), β-lipotropin, and β-endorphins. These hormones regulate hormonal secretions from the thyroid, pancreas, and adrenal glands, and act directly on the reproductive organs to slate ovulation and spermatogenesis. The posterior pituitary synthesizes and secretes antidiuretic hormone (ADH, vasopressin) and oxytocin.

Disorders of the hypothalamus and pituitary often result from lesions such as primary brain tumors, adenomas, infarction associated with pregnancy, hypophysectomy, aneurysms, vascular malformations, thrombosis, infections, immunological disorders, and complications due to head trauma. Such disorders have profound effects on the function of other endocrine glands. Disorders associated with hypopituitarism include hypogonadism, Sheehan syndrome, diabetes insipidus, Kallian's disease, Hand-Schuller-Christian disease, Letterer-Siwe disease, sarcoidosis, empty sella syndrome, and dwarfism Disorders associated with hyperpituitarism include acromegaly, giantism, and syndrome of inappropriate ADH secretion (SIADH), often caused by benign adenomas.

Hormones secreted by the thyroid and parathyroid primarily control metabolic rates and the regulation of serum calcium levels, respectively. Thyroid hormones include calcitonin, somatostatin, and thyroid hormone. The parathyroid secretes parathyroid hormone. Disorders associated with hypothyroidism include goiter, myxedema, acute thyroiditis associated with bacterial infection, subacute thyroiditis associated with viral infection, autoimmune thyroiditis (Hashimoto's disease), and cretinism Disorders associated with hyperthyroidism include thyrotoxicosis and its various forms, Grave's disease, pretibial myxedema, toxic multinodular goiter, thyroid carcinoma, and Plummer's disease. Disorders associated with hyperparathyroidism include Conn disease (chronic hypercalemia) leading to bone resorption and parathyroid hyperplasia.

Hormones secreted by the pancreas regulate blood glucose levels by modulating the rates of carbohydrate, fat, and protein metabolism. Pancreatic hormones include insulin, glucagon, amylin, γ-aminobutyric acid, gastrin, somatostatin, and pancreatic polypeptide. The principal disorder associated with pancreatic dysfunction is diabetes mellitus caused by insufficient insulin activity. Diabetes mellitus is generally classified as either Type I (insulin-dependent, juvenile diabetes) or Type II (non-insulin-dependent, adult diabetes). The treatment of both forms by insulin replacement therapy is well known. Diabetes mellitus often leads to acute complications such as hypoglycemia (insulin shock), coma, diabetic ketoacidosis, lactic acidosis, and chronic complications leading to disorders of the eye, kidney, skin, bone, joint, cardiovascular system, nervous system, and to decreased resistance to infection.

The anatomy, physiology, and diseases related to hormonal function are reviewed in McCance, K. L. and S. E. Huether (1994) Pathophysiology: The Biological Basis for Disease in Adults and Children, Mosby-Year Book, Inc., St Louis Mo.; Greenspan, F. S. and J. D. Baxter (1994) Basic and Clinical Endocrinology, Appleton and Lange, East Norwalk Conn.

Growth Factors

Growth factors are secreted proteins that mediate intercellular communication. Unlike hormones, which travel great distances via the circulatory system, most growth factors are primarily local mediators that act on neighboring cells. Most growth factors contain a hydrophobic N-terminal signal peptide sequence which directs the growth factor into the secretory pathway. Most growth factors also undergo post-translational modifications within the secretory pathway. These modifications can include proteolysis, glycosylation, phosphorylation, and intramolecular disulfide bond formation. Once secreted, growth factors bind to specific receptors on the surfaces of neighboring target cells, and the bound receptors trigger intracellular signal transduction pathways. These signal transduction pathways elicit specific cellular responses in the target cells. These responses can include the modulation of gene expression and the stimulation or inhibition of cell division, cell differentiation, and cell motility.

Growth factors fan into at least two broad and overlapping classes. The broadest class includes the large polypeptide growth factors, which are wide-ranging in their effects. These factors include epidermal growth factor (EGF), fibroblast growth factor (FGF), transforming growth factors (TGF-β), insulin-like growth factor (IGF), nerve growth factor (NGF), and platelet-derived growth factor (PDGF), each defining a family of numerous related factors. The large polypeptide growth factors, with the exception of NGF, act as mitogens on diverse cell types to stimulate wound healing, bone synthesis and remodeling, extracellular matrix synthesis, and proliferation of epithelial, epidermal, and connective tissues. Members of the TGF-β, EGF, and FGF families also function as inductive signals in the differentiation of embryonic tissue. NGF functions specifically as a neurotrophic factor, promoting neuronal growth and differentiation.

Another class of growth factors includes the hematopoietic growth factors, which are narrow in their target specificity. These factors stimulate the proliferation and differentiation of blood cells such as B-lymphocytes, T-lymphocytes, erythrocytes, platelets, eosinophils, basophils, neutrophils, macrophages, and their stem cell precursors. These factors include the colony-stimulating factors (G-CSF, M-CSF, GM-CSF, and CSF1-3), erythropoietin, and the cytokines. The cytokines are specialized hematopoietic factors secreted by cells of the immune system and are discussed in detail below.

Growth factors play critical roles in neoplastic transformation of cells in vitro and in tumor progression in vivo. Overexpression of the large polypeptide growth factors promotes the proliferation and transformation of cells in culture. Inappropriate expression of these growth factors by tumor cells in vivo may contribute to tumor vascularization and metastasis. Inappropriate activity of hematopoietic growth factors can result in anemias, leukemias, and lymphomas. Moreover, growth factors are both structurally and functionally related to oncoproteins, the potentially cancer-causing products of proto-oncogenes. Certain FGF and PDGF family members are themselves homologous to oncoproteins, whereas receptors for some members of the EGF, NGF, and FGF families are encoded by proto-oncogenes. Growth factors also affect the transcriptional regulation of both proto-oncogenes and oncosuppressor genes (Pimentel, E. (1994) Handbook of Growth Factors, CRC Press, Ann Arbor Mich.; McKay, I. and I. Leigh, eds. (1993) Growth Factors: A Practical Approach, Oxford University Press, New York N.Y.; Habenicht, A, ed. (1990) Growth Factors. Differentiation Factor, and Cytokines, Springer-Verlag, New York N.Y.).

In addition, some of the large polypeptide growth factors play crucial roles in the induction of the primordial germ layers in the developing embryo. This induction ultimately results in the formation of the embryonic mesoderm, ectoderm, and endoderm which in turn provide the framework for the entire adult body plan. Disruption of this inductive process would be catastrophic to embryonic development.

Small Peptide Factors—Neuropeptides and Vasomediators

Neuropeptides and vasomediators (NP/VM comprise a family of small peptide factors, typically of 20 amino acids or less. These factors generally function in neuronal excitation and inhibition of vasoconstriction/vasodilation, muscle contraction, and hormonal secretions from the brain and other endocrine tissues. Included in this family are neuropeptides and neuropeptide hormones such as bombesin, neuropeptide Y, neurotensin, neuromedin N, melanocortins, opioids, galanin, somatostatin, tachykinns, urotensin II and related peptides involved in smooth muscle stimulation, vasopressin, vasoactive intestinal peptide, and circulatory system-borne signaling molecules such as angiotensin, complement, calcitonin, endothelins, formyl-methionyl peptides, glucagon, cholecystokinin, gastrin, and many of the peptide hormones discussed above. NP/VMs can transduce signals directly, modulate the activity or release of other neurotransmitters and hormones, and act as catalytic enzymes in signaling cascades. The effects of NP/VMs range from extremely brief to long-lasting. (Reviewed in Martin, C. R. et al. (1985) Endocrine Physiology, Oxford University Press, New York N.Y., pp. 57-62.)

Cytokines

Cytokines comprise a family of signaling molecules that modulate the immune system and the inflammatory response. Cytokines are usually secreted by leukocytes, or white blood cells, in response to injury or infection. Cytokines function as growth and differentiation factors that act primarily on cells of the immune system such as B- and T-lymphocytes, monocytes, macrophages, and granulocytes. Like other signaling molecules, cytokines bind to specific plasma membrane receptors and trigger intracellular signal transduction pathways which alter gene expression patterns. There is considerable potential for the use of cytokines in the treatment of inflammation and immune system disorders.

Cytokine structure and function have been extensively characterized in vitro. Most cytokines are small polypeptides of about 30 kilodaltons or less. Over 50 cytokines have been identified from human and rodent sources. Examples of cytokine subfamilies include the interferons (IFN-α, -β, and -γ), the interleukins (IL1-IL13), the tumor necrosis factors (TNF-αand -β), and the chemokines. Many cytokines have been produced using recombinant DNA techniques, and the activities of individual cytokines have been determined in vitro. These activities include regulation of leukocyte proliferation, differentiation, and motility.

The activity of an individual cytokine in vitro may not reflect the full scope of that cytokine's activity in vivo. Cytokines are not expressed individually in vivo but are instead expressed in combination with a multitude of other cytokines when the organism is challenged with a stimulus. Together, these cytokines collectively modulate the immune response in a manner appropriate for that particular stimulus. Therefore, the physiological activity of a cytokine is determined by the stimulus itself and by complex interactive networks among co expressed cytokines which may demonstrate both synergistic and antagonistic relationships.

Chemokines comprise a cytokine subfamily with over 30 members. (Reviewed in Wells, T. N. C. and M. C. Peitsch (1997) J. Leukoc. Biol. 61:545-550.) Chemokines were initially identified as chemotactic proteins that recruit monocytes and macrophages to sites of inflammation Recent evidence indicates that chemokines may also play key roles in hematopoiesis and HIV-1 infection. Chemokines are small proteins which range from about 6-15 kilodaltons in molecular weight. Chemokines are further classified as C, CC, CXC, or CX₃C based on the number and position of critical cysteine residues. The CC chemokines, for example, each contain a conserved motif consisting of two consecutive cysteines followed by two additional cysteines which occur downstream at 24- and 16-residue intervals, respectively (ExPASy PROSITE database, documents PS00472 and PDOC00434). The presence and spacing of these four cysteine residues are highly conserved, whereas the intervening residues diverge significantly. However, a conserved tyrosine located about 15 residues downstream of the cysteine doublet seems to be important for chemotactic activity. Most of the human genes encoding CC chemokines are clustered on chromosome 17, although there are a few examples of CC chemokine genes that map elsewhere. Other chemokines include lymphotactin (C chemokine); macrophage chemotactic and activating factor (MCAF/MCP-1; CC chemokine); platelet factor 4 and IL-8 (CXC chemokines); and fractalkine and neurotractin (CX₃C chemokines). (Reviewed in Luster, A. D. (1998) N. Engl. J. Med. 338:436445.)

Receptor Molecules

The term receptor describes proteins that specifically recognize other molecules. The category is broad and includes proteins with a variety of functions. The bulk of receptors are cell surface proteins which bind extracellular ligands and produce cellular responses in the areas of growth, differentiation, endocytosis, and immune response. Other receptors facilitate the selective transport of proteins out of the endoplasmic reticulum and localize enzymes to particular locations in the cell. The term may also be applied to proteins which act as receptors for ligands with known or unknown chemical composition and which interact with other cellular components. For example, the steroid hormone receptors bind to and regulate transcription of DNA

Regulation of cell proliferation, differentiation, and migration is important for the formation and function of tissues. Regulatory proteins such as growth factors coordinately control these cellular processes and act as mediators in cell-cell signaling pathways. Growth factors are secreted proteins that bind to specific cell-surface receptors on target cells. The bound receptors trigger intracellular signal transduction pathways which activate various downstream effectors that regulate gene expression, cell division, cell differentiation, cell motility, and other cellular processes.

Cell surface receptors are typically integral plasma membrane proteins. These receptors recognize hormones such as catecholamines; peptide hormones; growth and differentiation factors; small peptide factors such as thyrotropin-releasing hormone; galanin, somatostatin, and tachykinins; and circulatory system-borne signaling molecules. Cell surface receptors on immune system cells recognize antigens, antibodies, and major histocompatibility complex (MHC)-bound peptides. Other cell surface receptors bind ligands to be internalized by the cell. This receptor-mediated edocytosis functions in the uptake of low density lipoproteins (LDL), transferrin, glucose- or mannose-terminal glycoproteins, galactose-terminal glycoproteins, immunoglobulins, phosphovitellogenins, fibrin, proteinase-inhibitor complexes, plasminogen activators, and thrombospondin (Lodish, H. et al. (1995) Molecular Cell Biology, Scientific American Books, New York N.Y., p. 723; Mikhailenko, I. et al. (1997) J. Biol. Chem. 272:67846791).

Receptor Protein Kinases

Many growth factor receptors, including receptors for epidermal growth factor, platelet-derived growth factor, fibroblast growth factor, as well as the growth modulator α-thrombin, contain intrinsic protein kinase activities. When growth factor binds to the receptor, it triggers the autophosphorylation of a serine, threonine, or tyrosine residue on the receptor. These phosphorylated sites are recognition sites for the binding of other cytoplasmic signaling proteins. These proteins participate in signaling pathways that eventually link the initial receptor activation at the cell surface to the activation of a specific intracellular target molecule. In the case of tyrosine residue autophosphorylation, these signaling proteins contain a common domain referred to as a Src homology (SH) domain. SH2 domains and SH3 domains are found in phospholipase C-γ, PI-3-K p85 regulatory subunit, Ras-GTPase activating protein, and pp60^{c-src} (Lowenstein, E. J. et al. (1992) Cell 70:431-442). The cytokine family of receptors share a different common binding domain and include transmembrane receptors for growth hormone (GM), interleukins, erythropoietin, and prolactin.

Other receptors and second messenger-binding proteins have intrinsic serine/threonine protein kinase activity. These include activin/TGF-β/BMP-superfamily receptors, calcium- and diacylglycerol-activated/phospolipid-dependent protein kinase (PK-C), and RNA ant protein kinase (PK-R). In addition, other serine/threonine protein kinases, including nematode Twitchin, have fibronectin-like, immunoglobulin C2-like domains.

G-Protein Coupled Receptors

G-protein coupled receptors (GPCRs) are integral membrane proteins characterized by the presence of seven hydrophobic transmembrane domains which span the plasma membrane and form a bundle of antiparallel alpha (a) helices. These proteins range in size from under 400 to over 1000 amino acids (Strosberg, A. D. (1991) Eur. J. Biochem. 196:1-10; Coughlin, S. R. (1994) Curr. Opin. Cell Biol. 6:191-197). The amino-terminus of the GPCR is extracellular, of variable length and often glycosylated; the carboxy-terminus is cytoplasmic and generally phosphorylated. Extracellular loops of the GPCR alternate with intracellular loops and link the transmembrane domains. The most conserved domains of GPCRs are the transmembrane domains and the first two cytoplasmic loops. The transmembrane domains account for structural and functional features of the receptor. In most cases, the bundle of a helices forms a binding pocket. In addition, the extracellular N-terminal segment or one or more of the three extracellular loops may also participate in ligand binding. Ligand binding activates the receptor by inducing a conformational change in intracellular portions of the receptor. The activated receptor, in turn, interacts with an intracellular heterotrimeric guanine nucleotide binding (G) protein complex which mediates further intracellular signing activities, generally the production of second messengers such as cyclic AMP (cAMP), phospholipase C, inositol triphosphate, or interactions with ion channel proteins (Baldwin, J. M. (1994) Curr. Opin. Cell Biol. 6:180-190).

GPCRs include those for acetylcholine, adenosine, epinephrine and norepinephrine, bombesin, bradykinin, chemokines, dopamine, endothelin, γ-aminobutyric acid (GABA), follicle-stimulating hormone (FSH), glutamate, gonadotropin-releasing hormone (GnRH), hepatocyte growth factor, histamine, leukotrienes, melanocortins, neuropeptide Y, opioid peptides, opsins, prostanoids, serotonin, somatostatin, tachykinins, thrombin, thyrotropin-releasing hormone (TRH), vasoactive intestinal polypeptide family, vasopressin and oxytocin, and orphan receptors.

GPCR mutations, which may cause loss of function or constitutive activation, have been associated with numerous human diseases (Coughlin, supra). For instance, retinitis pigmentosa may arise from mutations in the rhodopsin gene. Rhodopsin is the retinal photoreceptor which is located within the discs of the eye rod cell. Parma, J. et al. (1993, Nature 365:649-651) report that somatic activating mutations in the thyrotropin receptor cause hyperfunctioning thyroid adenomas and suggest that certain GPCRs susceptible to constitutive activation may behave as protooncogenes.

Nuclear Receptors

Nuclear receptors bind small molecules such as hormones or second messengers, leading to increased receptor-binding affinity to specific chromosomal DNA elements. In addition the affinity for other nuclear proteins may also be altered. Such binding and protein-protein interactions may regulate and modulate gene expression. Examples of such receptors include the steroid hormone receptors family, the retinoic acid receptors family, and the thyroid hormone receptors family.

Ligand-Gated Receptor Ion Channels

Ligand-gated receptor ion channels fall into two categories. The first category, exacellular ligand-gated receptor ion channels (ELGs), rapidly transduce neurotransmitter-binding events into electrical signals, such as fast synaptic neurotransmission. ELG function is regulated by post-translational modification. The second category, intracellular ligand-gated receptor ion channels (ILGs), are activated by many intracellular second messengers and do not require post-translational modification(s) to effect a channel-opening response.

ELGs depolarize excitable cells to the threshold of action potential generation. In non-excitable cells, ELGs permit a limited calcium ion-influx during the presence of agonist. ELGs include channels directly gated by neurotransmitters such as acetylcholine, L-glutamate, glycine, ATP, serotonin, GABA, and histamine. ELG genes encode proteins having strong structural and functional similarities. ILGs are encoded by distinct and unrelated gene families and include receptors for cAMP, cGMP, calcium ions, ATP, and metabolites of arachidonic acid

Macrophage Scavenger Receptors

Macrophage scavenger receptors with broad ligand specificity may participate in the binding of low density lipoproteins (LDL) and foreign antigens. Scavenger receptors types I and II are trimeric membrane proteins with each subunit containing a small N-terminal intracellular domain, a transmembrane domain, a large extracellular domain, and a C-terminal cysteine-rich domain. The extracellular domain contains a short spacer domain, an α-helical coiled-coil domain, and a triple helical collagenous domain. These receptors have been shown to bind a spectrum of ligands, including chemically modified lipoproteins and albumin, polyribonucleotides, polysaccharides, phospholipids, and asbestos (Matsumoto, A. et al. (1990) Proc. Natl. Acad. Sci. USA 87:9133-9137; Elomaa, 0. et al. (1995) Cell 80:603-609). The scavenger receptors are thought to play a key role in atherogenesis by mediating uptake of modified LDL in arterial walls, and in host defense by binding bacterial endotoxins, bacteria, and protozoa.

T-Cell Receptors

T cells play a dual role in the immune system as effectors and regulators, coupling antigen recognition with the transmission of signals that induce cell death in infected cells and stimulate proliferation of other immune cells. Although a population of T cells can recognize a wide range of different antigens, an individual T cell can only recognize a single antigen and only when it is presented to the T cell receptor (TCR) as a peptide complexed with a major histocompatibility molecule (MHC) on the surface of an antigen presenting cell. The TCR on most T cells consists of immunoglobulin-like integral membrane glycoproteins containing two polypeptide subunits, a and A, of similar molecular weight. Both TCR subunits have an extracellular domain containing both variable and constant regions, a transmembrane domain that traverses the membrane once, and a short intracellular domain (Saito, H. et al. (1984) Nature 309:757-762). The genes for the TCR subunits are constructed through somatic rearrangement of different gene segments. Interaction of antigen in the proper MHC context with the TCR initiates signaling cascades that induce the proliferation, maturation, and function of cellular components of the immune system (Weiss, A. (1991) Annu. Rev. Gene. 25:487-510). Rearrangements in TCR genes and alterations in TCR expression have been noted in lymphomas, leukemias, autoimmune disorders, and immunodeficiency disorders (Aisenberg, A. C. et al. (1985) N. Engl. J. Med. 313:529-533; Weiss, supra).

Intracellular Signaling Molecules

Intracellular signaling is the general process by which cells respond to extracellular signals (hormones, neurotransmitters, growth and differentiation factors, etc.) through a cascade of biochemical reactions that begins with the binding of a signaling molecule to a cell membrane receptor and ends with the activation of an intracellular target molecule. Intermediate steps in the process involve the activation of various cytoplasmic proteins by phosphorylation via protein kinases, and their deactivation by protein phosphatases, and the eventual translocation of some of these activated proteins to the cell nucleus where the transcription of specific genes is triggered. The intracellular signaling process regulates an types of cell functions including cell proliferation, cell differentiation, and gene transcription, and involves a diversity of molecules including protein kinases and phosphatases, and second messenger molecules, such as cyclic nucleotides, calcium-calmodulin, inositol, and various mitogens, that regulate protein phosphorylation.

Protein Phosphorylation

Protein kinases and phosphatases play a key role in the intracellular signaling process by controlling the phosphorylation and activation of various signaling proteins. The high energy phosphate for this reaction is generally transferred from the adenosine triphosphate molecule (ATP) to a particular protein by a protein kinase and removed from that protein by a protein phosphatase. Protein kinases are roughly divided into two groups: those that phosphorylate tyrosine residues (protein tyrosine kinases, PTK) and those that phosphorylate serine or threonine residues (serine/threonine kinases, STK). A few protein kinases have dual specificity for serine/threonine and tyrosine residues. Almost all kinases contain a conserved 250-300 amino acid catalytic domain containing specific residues and sequence motifs characteristic of the kinase family (Hardie, G. and S. Hanks (1995) The Protein Kinase Facts Books, Vol 1:7-20, Academic Press, San Diego Calif.).

STKs include the second messenger dependent protein kinases such as the cyclic-AMP dependent protein kinases (PKA), involved in mediating hormone-induced cellular responses; calcium-calmodulin (CaM) dependent protein kinases, involved in regulation of smooth muscle contraction, glycogen breakdown, and neurotransmission; and the mitogen-activated protein kinases (MAP) which mediate signal transduction from the cell surface to the nucleus via phosphorylation cascades. Altered PKA expression is implicated in a variety of disorders and diseases including cancer, thyroid disorders, diabetes, atherosclerosis, and cardiovascular disease (Isselbacher, K. J. et al. (1994) Harrison's Principles of Internal Medicine, McGraw-Hill, New York N.Y., pp. 416-431, 1887).

PTKs are divided into transmembrane, receptor PTKs and nontransmembrane, non-receptor PTKs. Transmembrane PTKs are receptors for most growth factors. Non-receptor PTKs lack transmembrane regions and, instead, form complexes with the intracellular regions of cell surface receptors. Receptors that function through non-receptor PTKs include those for cytokines and hormones (growth hormone and prolactin) and antigen-specific receptors on T and B lymphocytes. Many of these PTKs were first identified as the products of mutant oncogenes in cancer cells in which their activation was no longer subject to normal cellular controls. In fact, about one third of the known oncogenes encode PTKs, and it is well known that cellular transformation (oncogenesis) is often accompanied by increased tyrosine phosphorylation activity (Charbonneau, H. and N. K. Tonks (1992) Annu. Rev. Cell Biol. 8:463493).

An additional family of protein kinases previously thought to exist only in procaryotes is the histidine protein kinase family (HPK). BPKs bear little homology with mammalian STKs or PTKs but have distinctive sequence motifs of their own (Davie, J. R. et al. (1995) J. Biol. Chem. 270:19861-19867). A histidine residue in the N-terminal half of the molecule (region I) is an autophosphorylation site. Three additional motifs located in the C-terminal half of the molecule include an invariant asparagine residue in region II and two glycine-rich loops characteristic of nucleotide binding domains in regions III and IV. Recently a branched chain alpha-ketoacid dehydrogenase kinase has been found with characteristics of HPK in rat (Davie, supra).

Protein phosphatases regulate the effects of protein kinases by removing phosphate groups from molecules previously activated by kinases. The two principal categories of protein phosphatases are the protein (serine/threonine) phosphatases (PPs) and the protein tyrosine phosphatases (PTPs). PPs dephosphorylate phosphoserine/threonine residues and are important regulators of many cAMP-mediated hormone responses (Cohen, P. (1989) Annu. Rev. Biochem. 58:453-508). PTPs reverse the effects of protein tyrosine kinases and play a significant role in cell cycle and cell signaling processes (Charbonneau, supra). As previously noted, many PTKs are encoded by oncogenes, and oncogenesis is often accompanied by increased tyrosine phosphorylation activity. It is therefore possible that PTPs may prevent or reverse cell transformation and the growth of various cancers by controlling the levels of tyrosine phosphorylation in cells. This hypothesis is supported by studies showing that overexpression of PTPs can suppress transformation in cells, and that specific inhibition of PTPs can enhance cell transformation (Charbonneau, supra).

Phospholipid and Inositol-Phosohate Signaling

Inositol phospholipids (phosphoinositides) are involved in an intracellular signaling pathway that begins with binding of a signaling molecule to a G-protein linked receptor in the plasma membrane. This leads to the phosphorylation of phosphatidylinositol (PI) residues on the inner side of the plasma membrane to the biphosphate state (PIP₂) by inositol kinases. Simultaneously, the G-protein linked receptor binding stimulates a trimeric G-protein which in turn activates a phosphoinositide-specific phospholipase C-β. Phospholipase C-β then cleaves PIP₂ into two products, inositol triphosphate (IP₃) and diacylglycerol. These two products act as mediators for separate signaling events. IP₃ diffuses through the plasma membrane to induce calcium release from the endoplasmic reticulum (ER), while diacylglycerol remains in the membrane and helps activate protein kinase C, an STK that phosphorylates selected proteins in the target cell. The calcium response initiated by IP₃ is terminated by the dephosphorylation of IP₃ by specific inositol phosphatases. Cellular responses that are mediated by this pathway are glycogen breakdown in the liver in response to vasopressin, smooth muscle contraction in response to acetylcholine, and thrombin-induced platelet aggregation.

Cyclic Nucleotide Signaling

Cyclic nucleotides (cAMP and cGMP) function as intracellular second messengers to transduce a variety of extracellular signals including hormones, light, and neurotransmitters. In particular, cyclic-AMP dependent protein kinases (PKA) are thought to account for all of the effects of cAMP in most mammalian cells, including various hormone-induced cellular responses. Visual excitation and the phototransmission of light signals in the eye is controlled by cyclic-GMP regulated, Ca²⁺-specific channels. Because of the importance of cellular levels of cyclic nucleotides in mediating these various responses, regulating the synthesis and breakdown of cyclic nucleotides is an important matter. Thus adenylyl cyclase, which synthesizes cAMP from AMP, is activated to increase cAMP levels in muscle by binding of adrenaline to β-andrenergic receptors, while activation of guanylate cyclase and increased cGMP levels in photoreceptors leads to reopening of the Ca²⁺-specific channels and recovery of the dark state in the eye. In contrast, hydrolysis of cyclic nucleotides by cAMP and cGMP-specific phosphodiesterases (PDEs) produces the opposite of these and other effects mediated by increased cyclic nucleotide levels. PDEs appear to be particularly important in the regulation of cyclic nucleotides, considering the diversity found in this family of proteins. At least seven families of mammalian PDEs (PDE1-7) have been identified based on substrate specificity and affinity, sensitivity to cofactors, and sensitivity to inhibitory drugs (Beavo, J. A. (1995) Physiological Reviews 75:725-748). PDE inhibitors have been found to be particularly useful in treating various clinical disorders. Rolipram, a specific inhibitor of PDE4, has been used in the treatment of depression, and similar inhibitors are undergoing evaluation as anti-inflammatory agents. Theophylline is a nonspecific PDE inhibitor used in the treatment of bronchial asthma and other respiratory diseases (Banner, K. H. and C. P. Page (1995) Eur. Respir. J. 8:996-1000).

G-Protein Signaling

Guanine nucleotide binding proteins (G-proteins) are critical mediators of signal transduction between a particular class of extracelluar receptors, the G-protein coupled receptors (GPCR), and intracellular second messengers such as cAMP and Ca²⁺. G-proteins are linked to the cytosolic side of a GPCR such that activation of the GPCR by ligand binding stimulates binding of the G-protein to GTP, inducing an “active” state in the G-protein. In the active state, the G-protein acts as a signal to trigger other events in the cell such as the increase of cAMP levels or the release of Ca²⁺ into the cytosol from the ER, which, in turn, regulate phosphorylation and activation of other intracellular proteins. Recycling of the G-protein to the inactive state involves hydrolysis of the bound GTP to GDP by a GTPase activity in the G-protein. (See Alberts, B. et al. (1994) Molecular Biology of the Cell, Garland Publishing, Inc., New York N.Y., pp.734-759.) Two structurally distinct classes of G-proteins are recognized: heterotrimeric G-proteins, consisting of three different subunits, and monomeric, low molecular weight (LMW), G-proteins consisting of a single polypeptide chain.

The three polypeptide subunits of heterotrimeric G-proteins are the α, β, and γ subunits. The α subunit binds and hydrolyzes GTP. The β and γ subunits form a tight complex that anchors the protein to the inner side of the plasma membrane. The β subunits, also known as G-β proteins or β transducins, contain seven tandem repeats of the WD-repeat sequence motif, a motif found in many proteins with regulatory functions. Mutations and variant expression of β transducin proteins are linked with various disorders (Neer, E. J. et al. (1994) Nature 371:297-300; Margottin, F. et al. (1998) Mol. Cell 1:565-574).

LMW GTP-proteins are GTPases which regulate cell growth, cell cycle control, protein secretion, and intracellular vesicle interaction. They consist of single polypeptides which, like the a subunit of the heterotrimeric G-proteins, are able to bind and hydrolyze GTP, thus cycling between an inactive and an active state. At least sixty members of the LMW G-protein superfamily have been identified and are currently grouped into the six subfamilies of ras, rho, arf, sar1, ran, and rab. Activated ras genes were initially found in human cancers, and subsequent studies conformed that ras function is critical in determining whether cells continue to grow or become differentiated. Other members of the LMW G-protein superfamily have roles in signal transduction that vary with the function of the activated genes and the locations of the G-proteins.

Guanine nucleotide exchange factors regulate the activities of LMW G-proteins by determining whether GTP or GDP is bound. GTPase-activating protein (GAP) binds to GTP-ras and induces it to hydrolyze GTP to GDP. In contrast, guanine nucleotide releasing protein (GNRP) binds to GDP-ras and induces the release of GDP and the binding of GTP.

Other regulators of G-protein signaling (RGS) also exist that act primarily by negatively regulating the G-protein pathway by an unknown mechanism (Druey, KM. et al. (1996) Nature 379:742-746). Some 15 members of the RGS family have been identified. RGS family members are related structurally through similarities in an approximately 120 amino acid region termed the RGS domain and functionally by their ability to inhibit the interleukin (cytokine) induction of MAP kinase in cultured mammalian 293T cells (Druey, supra).

Calcium Signaling Molecules

Ca⁺² is another second messenger molecule that is even more widely used as an intracellular mediator than cAMP. Two pathways exist by which Ca⁺² can enter the cytosol in response to extracellular signals: One pathway acts primarily in nerve signal transduction where Ca⁺² enters a nerve terminal through a voltage-gated Ca⁺² channel. The second is a more ubiquitous pathway in which Ca⁺² is released from the ER into the cytosol in response to binding of an extracellular signaling molecule to a receptor. Ca²⁺ directly activates regulatory enzymes, such as protein kinase C, which trigger signal transduction pathways. Ca²⁺ also binds to specific Ca²⁺-binding proteins (CBPs) such as calmodulin (CaM) which then activate multiple target proteins in the cell including enzymes, membrane transport pumps, and ion channels. CaM interactions are involved in a multitude of cellular processes including, but not limited to, gene regulation, DNA synthesis, cell cycle progression, mitosis, cytokinesis, cytoskeletal organization, muscle contraction, signal transduction, ion homeostasis, exocytosis, and metabolic regulation (Celio, M. R. et al. (1996) Guidebook to Calcium-binding Proteins, Oxford University Press, Oxford, UK, pp. 15-20). Some CBPs can serve as a storage depot for Ca²⁺ in an inactive state. Calsequestrin is one such CBP that is expressed in isoforms specific to cardiac muscle and skeletal muscle. It is suggested that calsequestrin binds Ca²⁺ in a rapidly exchangeable state that is released during Ca²⁺-signaling conditions (Celio, M. R. et al. (1996) Guidebook to Calcium-binding Proteins, Oxford University Press, New York N.Y., pp. 222-224).

Cyclins

Cell division is the fundamental process by which all living things grow and reproduce. In most organisms, the cell cycle consists of three principle steps; interphase, mitosis, and cytokinesis. Interphase, involves preparations for cell division, replication of the DNA and production of essential proteins. In mitosis, the nuclear material is divided and separates to opposite sides of the cell. Cytokinesis is the final division and fission of the cell cytoplasm to produce the daughter cells.

The entry and exit of a cell from mitosis is regulated by the synthesis and destruction of a family of activating proteins called cyclins. Cyclins act by binding to and activating a group of cyclin-dependent protein kinases (Cdks) which then phosphorylate and activate selected proteins involved in the mitotic process. Several types of cyclins exist (Ciechanover, A (1994) Cell 79:13-21.) Two principle types are mitotic cyclin, or cyclin B, which controls entry of the cell into mitosis, and G1 cyclin, which controls events that drive the cell out of mitosis.

Signal Complex Scaffolding Proteins

Ceretain proteins in intracellular signaling pathways serve to link or cluster other proteins involved in the signaling cascade. A conserved protein domain called the PDZ domain has been identified in various membrane-associated signaling proteins. This domain has been implicated in receptor and ion channel clustering and in the targeting of multiprotein signaling complexes to specialized functional regions of the cytosolic face of the plasma membrane. (For a review of PDZ domain-containing proteins, see Ponting, C. P. et al. (1997) Bioessays 19:469-479.) A large proportion of PDZ domains are found in the eukaryotic MAGUK (membrane-associated guanylate kinase) protein family, members of which bind to the intracellular domains of receptors and channels. However, PDZ domains are also found in diverse membrane-localized proteins such as protein tyrosine phosphatases, serine/threonine kinases, G-protein cofactors, and synapse-associated proteins such as syntrophins and neuronal nitric oxide synthase (nNOS). Generally, about one to three PDZ domains are found in a given protein, although up to nine PDZ domains have been identified in a single protein.

Membrane Transport Molecules

The plasma membrane acts as a barrier to most molecules. Transport between the cytoplasm and the extracellular environment, and between the cytoplasm and lumenal spaces of cellular organelles requires specific transport proteins. Each transport protein carries a particular class of molecule, such as ions, sugars, or amino acids, and often is specific to a certain molecular species of the class. A variety of human inherited diseases are caused by a mutation in a transport protein. For example, cystinuria is an inherited disease that results from the inability to transport cystine, the disulfide-linked dimer of cysteine, from the urine into the blood Accumulation of cystine in the urine leads to the formation of cystine stones in the kidneys.

Transport proteins are multi-pass transmembrane proteins, which either actively transport molecules across the membrane or passively allow them to cross. Active transport involves directional pumping of a solute across the membrane, usually against an electrochemical gradient Active transport is tightly coupled to a source of metabolic energy, such as ATP hydrolysis or an electrochemically favorable ion gradient. Passive transport involves the movement of a solute down its electrochemical gradient. Transport proteins can be further classified as either carrier proteins or channel proteins. Carrier proteins, which can function in active or passive transport, bind to a specific solute to be transported and undergo a conformational change which transfers the bound solute across the membrane. Channel proteins, which only function in passive transport, form hydrophilic pores across the membrane. When the pores open, specific solutes, such as inorganic ions, pass through the membrane and down the electrochemical gradient of the solute.

Carrier proteins which transport a single solute from one side of the membrane to the other are called uniporters. In contrast, coupled transporters link the transfer of one solute with simultaneous or sequential transfer of a second solute, either in the same direction (symport) or in the opposite direction (antiport). For example, intestinal and kidney epithelium contains a variety of symporter systems driven by the sodium gradient that exists across the plasma membrane. Sodium moves into the cell down its electrochemical gradient and brings the solute into the cell with it. The sodium gradient that provides the driving force for solute uptake is maintained by the ubiquitous Na⁺/K⁺ ATPase. Sodium-coupled transporters include the mammalian glucose transporter (SGLT1), iodide transporter (NIS), and multivitamin transporter (SMVT). All three transporters have twelve putative transmembrane segments, extracellular glycosylation sites, and cytoplasmically-oriented N- and C-termini. NIS plays a crucial role in the evaluation, diagnosis, and treatment of various thyroid pathologies because it is the molecular basis for radioiodide thyroid-imaging techniques and for specific targeting of radioisotopes to the thyroid gland (Levy, O. et al. (1997) Proc. Natl. Acad. Sci. USA 94:5568-5573). SMVT is expressed in the intestinal mucosa, kidney, and placenta, and is implicated in the transport of the water-soluble vitamins, e.g., biotin and pantothenate (Prasad, P. D. et al. (1998) J. Biol. Chem. 273:7501-7506).

Transporters play a major role in the regulation of pH, excretion of drugs, and the cellular K⁺/Na⁺ balance. Monocarboxylate anion transporters are proton-coupled symporters with a broad substrate specificity that includes L-lactate, pyruvate, and the ketone bodies acetate, acetoacetate, and beta-hydroxybutyrate. At least seven isoforms have been identified to date. The isoforms are predicted to have twelve transmembrane (TM) helical domains with a large intracellular loop between TM6 and TM7, and play a critical role in maintaining intracellular pH by removing the protons that are produced stoichiometrically with lactate during glycolysis. The best characterized H(+)-monocarboxylate transporter is that of the erythrocyte membrane, which transports L-lactate and a wide range of other aliphatic monocarboxylates. Other cells possess H(+)-linked monocarboxylate transporters with differing substrate and inhibitor selectivities. In particular, cardiac muscle and tumor cells have transporters that differ in their Kₘ values for certain substrates, including stereoselectivity for L-over D-lactate, and in their sensitivity to inhibitors. There are Na(+)-monocarboxylate cotransporters on the luminal surface of intestinal and kidney epithelia, which allow the uptake of lactate, pyruvate, and ketone bodies in these tissues. In addition, there are specific and selective transporters for organic cations and organic anions in organs including the kidney, intestine and liver. Organic anion transporters are selective for hydrophobic, charged molecules with electron-attracting side groups. Organic cation transporters, such as the ammonium transporter, mediate the secretion of a variety of drugs and endogenous metabolites, and contribute to the maintenance of intercellular pH. (Poole, R. C. and A. P. Halestrap (1993) Am J. Physiol. 264:C761-C782; Price, N. T. et al. (1998) Biochnol. J. 329:321-328; and Martinelle, Ky. and I. Haggstrom (1993) J. Biotechnol. 30: 339-350.)

The largest and most diverse family of transport proteins known is the ATP-binding cassette (ABC) transporters. As a family, ABC transporters can transport substances that differ markedly in chemical structure and size, ranging from small molecules such as ions, sugars, amino acids, peptides, and phospholipids, to lipopeptides, large proteins, and complex hydrophobic drugs. ABC proteins consist of four modules: two nucleotide-binding domains (NBD), which hydrolyze ATP to supply the energy required for transport, and two membrane-spanning domains (MSD), each containing six putative transmembrane segments. These four modules may be encoded by a single gene, as is the case for the cystic fibrosis transmembrane regulator (CFTR), or by separate genes. When encoded by separate genes, each gene product contains a single NBD and MSD. These “half-molecales” form homo- and heterodimers, such as Tap1 and Tap2, the endoplasmic reticulum-based major histocompatibility (MHC) peptide transport system. Several genetic diseases are attributed to defects in ABC transporters, such as the following diseases and their corresponding proteins: cystic fibrosis (CFTR, an ion channel), adrenoleukodystrophy (adrenoleukodystrophy protein, ALDP), Zellweger syndrome (peroxisomal membrane protein-70, PMP70), and hyperinsulinemic hypoglycemia (sulfonylurea receptor, SUR). Overexpression of the multidrug resistance (MDR) protein, another ABC transporter, in human cancer cells makes the cells resistant to a variety of cytotoxic drugs used in chemotherapy (Taglight, D. and S. Michaelis (1998) Meth. Enzymol. 292:131-163).

Transport of fatty acids across the plasma membrane can occur by diffusion, a high capacity, low affinity process. However, under normal physiological conditions a significant fraction of fatty acid transport appears to occur via a high affinity, low capacity protein-mediated transport process. Fatty acid transport protein (FATP), an integral membrane protein with four transmembrane segments, is expressed in tissues exhibiting high levels of plasma membrane fatty acid flux, such as muscle, heart, and adipose. Expression of FATP is upregulated in 3T3-L1 cells during adipose conversion, and expression in COS7 fibroblasts elevates uptake of long-chain fatty acids (Hui, T. Y. et al. (1998) J. Biol. Chem. 273:27420-27429).

Ion Channels

The electrical potential of a cell is generated and maintained by controlling the movement of ions across the plasma membrane. The movement of ions requires ion channels, which form an ion-selective pore within the membrane. There are two basic types of ion channels, ion transporters and gated ion channels. Ion transporters utilize the energy obtained from ATP hydrolysis to actively transport an ion against the ion's concentration gradient Gated ion channels allow passive flow of an ion down the ion's electrochemical gradient under restricted conditions. Together, these types of ion channels generate, maintain, and utilize an electrochemical gradient that is used in 1) electrical impulse conduction down the axon of a nerve cell, 2) transport of molecules into cells against concentration gradients, 3) initiation of muscle contraction, and 4) endocrine cell secretion.

Ion transporters generate and maintain the resting electrical potential of a cell. Utilizing the energy derived from ATP hydrolysis, they transport ions against the ion's concentration gradient. These transmembrane ATPases are divided into three families. The phosphorylated (P) class ion transporters, including Na⁺-K⁺ ATPase, Ca²⁺-ATPase, and H⁺-ATPase, are activated by a phosphorylation event. P-class ion transporters are responsible for maintaining resting potential distributions such that cytosolic concentrations of Na⁺ and Ca⁺ are low and cytosolic concentration of K⁺ is high. The vacuolar (V) class of ion transporters includes H⁺ pumps on intracellular organelles, such as lysosomes and Golgi. V-class ion transporters are responsible for generating the low pH within the lumen of these organelles that is required for function. The coupling factor (F) class consists of H⁺ pumps in the mitochondria. F-class ion transporters utilize a proton gradient to generate ATP from ADP and inorganic phosphate (Pᵢ).

The resting potential of the cell is utilized in many processes involving carrier proteins and gated ion channels. Carrier proteins utilize the resting potential to transport molecules into and out of the cell. Amino acid and glucose transport into many cells is linked to sodium ion co-transport (symport) so that the movement of Na⁺ down an electrochemical gradient drives transport of the other molecule up a concentration gradient. Similarly, cardiac muscle links transfer of Ca²⁺ out of the cell with transport of Nat into the cell (antiport).

Ion channels share common structural and mechanistic themes. The channel consists of four or five subunits or protein monomers that are arranged like a barrel in the plasma membrane. Each subunit typically consists of six potential transmembrane segments (S1, S2, S3, S4, S5, and S6). The center of the barrel forms a pore lined by α-helices or β-strands. The side chains of the amino acid residues comprising the α-helices or β-strands establish the charge (cation or anion) selectivity of the channel. The degree of selectivity, or what specific ions are allowed to pass through the channel, depends on the diameter of the narrowest part of the pore.

Gated ion channels control ion flow by regulating the opening and closing of pores. These channels are categorized according to the manner of regulating the gating function. Mechanically-gated channels open pores in response to mechanical stress, voltage-gated channels open pores in response to changes in membrane potential, and ligand-gated channels open pores in the presence of a specific ion, nucleotide, or neurotransmitter.

Voltage-gated Na⁺ and K⁺ channels are necessary for the function of electrically excitable cells, such as nerve and muscle cells. Action potentials, which lead to neurotransmittter release and muscle contraction, arise from large, transient changes in the permeability of the membrane to Na⁺ and K⁺ ions. Depolarization of the membrane beyond the threshold level opens voltage-gated Na⁺ channels. Sodium ions flow into the cell, further depolarizing the membrane and opening more voltage-gated Na⁺ channels, which propagates the depolarization down the length of the cell. Depolarization also opens voltage-gated potassium channels. Consequently, potassium ions flow outward, which leads to repolarization of the membrane. Voltage-gated channels utilize charged residues in the fourth transmembrane segment (S4) to sense voltage change. The open state lasts only about 1 millisecond, at which tire the channel spontaneously converts into an inactive state that cannot be opened irrespective of the membrane potential. Inactivation is mediated by the channel's N-terminus, which acts as a plug that closes the pore. The transition from an inactive to a closed state requires a return to resting potential.

Voltage-gated Na⁺ channels are heterotrimeric complexes composed of a 260 kDa pore forming α subunit that associates with two smaller auxiliary subunits, β1 and β2. The β2 subunit is an integral membrane glycoprotein that contains an extracellular Ig domain, and its association with a and β1 subunits correlates with increased functional expression of the channel, a change in its gating properties, and an increase in whole cell capacitance due to an increase in membrane surface area. (Isom, L. L. et al. (1995) Cell 83:433442.)

Voltage-gated Ca²⁺ channels are involved in presynaptic neurotransmitter release, and heart and skeletal muscle contraction. The voltage-gated Ca²⁺ channels from skeletal muscle (L-type) and brain (N-type) have been purified, and though their functions differ dramatically, they have similar subunit compositions. The channels are composed of three subunits. The a, subunit forms the membrane pore and voltage sensor, while the α₂δ and β subunits modulate the voltage-dependence, gating properties, and the current amplitude of the channel. These subunits are encoded by at least six α₁, one α₂δ, and four β genes. A fourth subunit, γ, has been identified in skeletal muscle. (Walker, D. et al. (1998) J. Biol. Chem. 273:2361-2367; and Jay, S. D. et al. (1990) Science 248:490-492.)

Chloride channels are necessary in endocrine secretion and in regulation of cytosolic and organelle pH. In secretory epithelial cells, Cl⁻ enters the cell across a basolateral membrane through an Na⁺, K⁺/Cl⁻ cotransporter, accumulating in the cell above its electrochemical equilibrium concentration. Secretion of Cl⁻ from the apical surface, in response to hormonal stimulation, leads to flow of Na⁺ and water into the secretory lumen. The cystic fibrosis transmembrane conductance regulator (CFTR) is a chloride channel encoded by the gene for cystic fibrosis, a common fatal genetic disorder in humans. Loss of CFTR function decreases transepithelial water secretion and, as a result, the layers of mucus that coat the respiratory tree, pancreatic ducts, and intestine are dehydrated and difficult to clear. The resulting blockage of these sites leads to pancreatic insufficiency, “meconium ileus”, and devastating “chronic obstructive pulmonary disease” (AI-Awqati, Q. et al. (1992) J. Exp. Biol. 172:245-266).

Many intracellular organelles contain H⁺-ATPase pumps that generate transmembrane pH and electrochemical differences by moving protons from the cytosol to the organelle lump. If the membrane of the organelle is permeable to other ions, then the electrochemical gradient can be abrogated without affecting the pH differential. In fact, removal of the electrochemical barrier allows more H⁺ to be pumped across the membrane, increasing the pH differential. Cl⁻ is the sole counterion of H⁺ translocation in a number of organelles, including chromaffin granules, Golgi vesicles, lysosomes, and endosomes. Functions that require a low vacuolar pH include uptake of small molecules such as biogenic amines in chromaffin granules, processing of vacuolar constituents such as pro-hormones by proteolytic enzymes, and protein degradation in lysosomes (Al-Awqati, supra).

Ligand-gated channels open their pores when an extracellular or intracellular mediator binds to the channel. Neurotransmitter-gated channels are channels that open when a neurotransmitter binds to their extracellular domain. These channels exist in the postsynaptic membrane of nerve or muscle cells. There are two types of neurotransmitter-gated channels. Sodium channels open in response to excitatory neurotransmitters, such as acetylcholine, glutamate, and serotonin. This opening causes an influx of Na⁺ and produces the initial localized depolarization that activates the voltage-gated channels and starts the action potential. Chloride channels open in response to inhibitory neurotransmitters, such as γ-aminobutyric acid (GABA) and glycine, leading to hyperpolarization of the membrane and the subsequent generation of an action potential.

Ligand-gated channels can be regulated by intracellular second messengers. Calcium-activated K⁺ channels are gated by internal calcium ions. In nerve cells, an influx of calcium during depolarization opens K⁺ channels to modulate the magnitude of the action potential ([shi, T. M. et al. (1997) Proc. Natl. Acad. Sci. USA 94:11651-11656). Cyclic nucleotide-gated (CNG) channels are gated by cytosolic cyclic nucleotides. The best examples of these are the cAMP-gated Na⁺ channels involved in olfaction and the cGMP-gated cation channels involved in vision. Both systems involve ligand-mediated activation of a G-protein coupled receptor which then alters the level of cyclic nucleotide within the cell.

Ion channels are expressed in a number of tissues where they are implicated in a variety of processes. CNG channels, while abundantly expressed in photoreceptor and olfactory sensory cells, are also found in kidney, lung, pineal, retinal ganglion cells, testis, aorta, and brain. Calcium-activated K⁺ channels may be responsible for the vasodilatory effects of bradykinin in the kidney and for shunting excess K⁺ from brain capillary endothelial cells into the blood. They are also implicated in repolarizing granulocytes after agonist-stimulated depolarization (Ishi, supra). Ion channels have been the target for many drug therapies. Neurotransmitter-gated channels have been targeted in therapies for treatment of insomnia, anxiety, depression, and schizophrenia. Voltage-gated channels have been targeted in therapies for arrhythmia, ischemic stroke, head trauma, and neurodegenerative disease (Taylor, C. P. and L. S. Narasimhan (1997) Adv. Pharmacol. 39:47-98).

Disease Correlation

The etiology of numerous human diseases and disorders can be attributed to defects in the transport of molecules across membranes. Defects in the trafficking of membrane-bound transporters and ion channels are associated with several disorders, e.g. cystic fibrosis, glucose-galactose malabsorption syndrome, hypercholesterolemia, von Gierke disease, and certain forms of diabetes mellitus. Single-gene defect diseases resulting in an inability to transport small molecules across membranes include, e.g., cystinuria, iminoglycinuria, Hartup disease, and Fanconi disease (van't Hoff, W. G. (1996) Exp. Nephrol. 4:253-262; Talente, G. M. et al. (1994) Ann. Intern. Med. 120:218-226; and Chillon, M. et al. (1995) New Engl. J. Med. 332:1475-1480).

Protein Modification and Maintenance Molecules

The cellular processes regulating modification and maintenance of protein molecules coordinate their conformation, stabilization, and degradation. Each of these processes is mediated by key enzymes or proteins such as proteases, protease inhibitors, transferases, isomerases, and molecular chaperones.

Proteases

Proteases cleave proteins and peptides at the peptide bond that forms the backbone of the peptide and protein chain Proteolytic processing is essential to cell growth, differentiation, remodeling, and homeostasis as well as inflammation and immune response. Typical protein half-lives range from hours to a few days, so that within all living cells, precursor proteins are being cleaved to their active form, signal sequences proteolytically removed from targeted proteins, and aged or defective proteins degraded by proteolysis. Proteases function in bacterial, parasitic, and viral invasion and replication within a host. Four principal categories of mammalian proteases have been identified based on active site structure, mechanism of action, and overall three-dimensional structure. (Beynon, R. J. and J. S. Bond (1994) Proteolytic Enzymes: A Practical Approach, Oxford University Press, New York N.Y., pp. 1-5).

The serine proteases (SPs) have a serine residue, usually within a conserved sequence, in an active site composed of the serine, an aspartate, and a histidine residue. SPs include the digestive enzymes trypsin and chymotrypsin, components of the complement cascade and the blood-clotting cascade, and enzymes that control extracellular protein degradation. The main SP sub-families are trypases, which cleave after arginine or lysine; aspartases, which cleave after aspartate; chymases, which cleave after phenylalanine or leucine; metases, which cleavage after methionine; and serases which cleave after serine. Enterokinase, the initiator of intestinal digestion, is a serine protease found in the intestinal brush border, where it cleaves the acidic propeptide from trypsinogen to yield active trypsin (Kitamoto, Y. et al. (1994) Proc. Natl. Acad. Sci. USA 91:7588-7592). Prolylcarboxypeptidase, a lysosomal serine peptidase that cleaves peptides such as angiotensin II and III and [des-Arg9] bradykinin, shares sequence homology with members of both the serine carboxypeptidase and prolylendopeptidase families (Tan, F. et al. (1993) J. Biol. Chem. 268:16631-16638).

Cysteine proteases (CPs) have a cysteine as the major catalytic residue at an active site where catalysis proceeds via an intermediate thiol ester and is facilitated by adjacent histidine and aspartic acid residues. CPs are involved in diverse cellular processes ranging from the processing of precursor proteins to intracellular degradation. Mammalian CPs include lysosomal cathepsins and cytosolic calcium activated proteases, calpains. CPs are produced by monocytes, macrophages and other cells of the immune system which migrate to sites of inflammation and secrete molecules involved in tissue repair. Overabundance of these repair molecules plays a role in certain disorders. In autoimmune diseases such as rheumatoid arthritis, secretion of the cysteine peptidase cathepsin C degrades collagen, laminin, elastin and other structural proteins found in the extracellular matrix of bones.

Aspartic proteases are members of the cathepsin family of lysosomal proteases and include pepsin A, gastricsin, chymosin, renin, and cathepsins D and E. Aspartic proteases have a pair of aspartic acid residues in the active site, and are most active in the pH 2-3 range, in which one of the aspartate residues is ionized, the other un-ionized. Aspartic proteases include bacterial penicillopepsin, mammalian pepsin, renin, chymosin, and certain fungal proteases. Abnormal regulation and expression of cathepsins is evident in various inflammatory disease states. In cells isolated from inflamed synovia, the mRNA for stromelysin, cytokines, TEMP-1, cathepsin, gelatinase, and other molecules is preferentially expressed. Expression of cathepsins L and D is elevated in synovial tissues from patients with rheumatoid arthritis and osteoarthritis. Cathepsin L expression may also contribute to the influx of mononuclear cells which exacerbates the, destruction of the rheumatoid synovium (Keyszer, G. M. (1995) Arthritis Rheum. 38:976-984.) The increased expression and differential regulation of the, cathepsins are linked to the metastatic potential of a variety of cancers and as such are of therapeutic and prognostic interest (Chambers, A. F. et al. (1993) Crit. Rev. Oncog. 4:95-114).

Metalloproteaes have active sites that include two glutamic acid residues and one histidine residue that serve as binding sites for Zinc. Carboxypeptidases A and B are the principal mammalian metalloproteases. Both are exoproteases of similar structure and active sites. Carboxypeptidase A, like chymotrypsin prefers C-terminal aromatic and aliphatic side chains of hydrophobic nature, whereas carboxypeptidase B is directed toward basic arginine and lysine residues. Glycoprotease (GCP), or O-sialoglycoprotein endopeptidase, is a metallopeptidase which specifically cleaves O-sialoglycoproteins such as glycophorin A. Another metallopeptidase, placental leucine aminopeptidase (P-LAP) degrades several peptide hormones such as oxytocin and vasopressin, suggesting a role in maintaining homeostasis during pregnancy, and is expressed in several tissues (Rogi, T. et al. (1996) J. Biol. Chem. 271:56-61).

Ubiquitin proteases are associated with the ubiquitin conjugation system (UCS), a major pathway for the degradation of cellular proteins in eukaryotic cells and some bacteria. The UCS mediates the elimination of abnormal proteins and regulates the half-lives of important regulatory proteins that control cellular processes such as gene transcription and cell cycle progression. In the UCS pathway, proteins targeted for degradation are conjugated to a ubiquitin, a small heat stable protein. The ubiquitinated protein is then recognized and degraded by proteasome, a large, multisubunit proteolytic enzyme complex, and ubiquitin is released for reutilization by ubiquitin protease. The UCS is implicated in the degradation of mitotic cyclic kinases, oncoproteins, tumor suppressor genes such as p53, viral proteins, cell surface receptors associate with signal transduction, transcriptional regulators, and mutated or damaged proteins (Ciechanover, k (1994) Cell 79:13-21). A murine proto-oncogene, Unp, encodes a nuclear ubiquitin protease whose overexpression leads to oncogenic transformation of NIH3T3 cells, and the human homolog of this gene is consistently elevated in small cell tumors and adenocarcinomas of the lung (Gray, D. A. (1995) Oncogene 10:2179-2183).

Signal Peptidases

The mechanism for the translocation process into the endoplasmic reticulum (ER) involves the recognition of an N-terminal signal peptide on the elongating protein. The signal peptide direct the protein and attached ribosome to a receptor on the ER membrane. The polypeptide chain passes through a pore in the ER membrane into the lumen while the N-terminal signal peptide remains attached at the membrane surface. The process is completed when signal peptidase located inside the ER cleaves the signal peptide from the protein and releases the protein into the lumen.

Protease Inhibitors

Protease inhibitors and other regulators of protease activity control the activity and effects of proteases. Protease inhibitors have been shown to control pathogenesis in animal models of proteolytic disorders (Murphy, G. (1991) Agents Actions Suppl. 35:69-76). Low levels of the cystatins, low molecular weight inhibitors of the cysteine proteases, correlate with malignant progression of tumors. (Calkins, C. et al (1995) Biol. Biochem. Hoppe Seyler 376:71-80). Serpins are inhibitors of mammalian plasma serine proteases. Many serpins serve to regulate the blood clotting cascade and/or the complement cascade in mammals. Sp32 is a positive regulator of the mammalian acrosomal protease, acrosin, that binds the proenzyme, proacrosin, and thereby aides in packaging the enzyme into the acrosomnal matrix (Baba, T. et al. (1994) 1. Biol. Chem. 269:10133-10140). The Kunitz family of serine protease inhibitors are characterized by one or more “Kunitz domains” containing a series of cysteine residues that are regularly spaced over approximately 50 amino acid residues and form three intrachain disulfide bonds. Members of this family include aprotinin, tissue factor pathway inhibitor (TFPI-1 and TFPI-2), inter-α-trypsin inhibitor, and bikunin. (Marlor, C. W. et al. (1997) J. Biol. Chem. 272:12202-12208.) Members of this family are potent inhibitors (in the nanomolar range) against serine proteases such as kallikrein and plasmin. Aprotinin has clinical utility in reduction of perioperative blood loss.

A major portion of all proteins synthesized in eukaryotic cells are synthesized on the cytosolic surface of the endoplasmic reticulum (ER). Before these immature proteins are distributed to other organelles in the cell or are secreted, they must be transported into the interior lumen of the ER where post-translational modifications are performed. These modifications include protein folding and the formation of disulfide bonds, and N-linked glycosylations.

Protein Isomerases

Protein folding in the ER is aided by two principal types of protein isomerases, protein disulfide isomerase (PDI), and peptidyl-prolyl isomerase (PPI). PDI catalyzes the oxidation of free sulfhydryl groups in cysteine residues to form intramolecular disulfide bonds in proteins. PPI, an enzyme that catalyzes the isomerization of certain proline imidic bonds in oligopeptides and proteins, is considered to govern one of the rate limiting steps in the folding of many proteins to their final functional conformation. The cyclophilins represent a major class of PPI that was originally identified as the major receptor for the immunosuppressive drug cyclosporin A (Handschumacher, R. E. et al. (1984) Science 226: 544-547).

Protein Glycosylation

The glycosylation of most soluble secreted and membrane-bound proteins by oligosaccharides linked to asparagine residues in proteins is also performed in the ER. This reaction is catalyzed by a membrane-bound enzyme, oligosaccharyl transferase. Although the exact purpose of this “N-linked” glycosylation is unknown, the presence of oligosaccharides tends to make a glycoprotein resistant to protease digestion. In addition, oligosaccharides attached to cell-surface proteins called selectins are known to function in cell-cell adhesion processes (Alberts, B. et al. (1994) Molecular Biology of the Cell, Garland Publishing Co., New York N.Y., p.608). “O-linked” glycosylation of proteins also occurs in the ER by the addition of N-acetylgalactosamine to the hydroxyl group of a serine or threonine residue followed by the sequential addition of other sugar residues to the first. This process is catalysed by a series of glycosyltransferases each specific for a particular donor sugar nucleotide and acceptor molecule (Lodish, H. et al. (1995) Molecular Cell Biology, W. H. Freeman and Co., New York N.Y., pp.700-708). In many cases, both N- and O-linked oligosaccharides appear to be required for the secretion of proteins or the movement of plasma membrane glycoproteins to the cell surface.

An additional glycosylation mechanism operates in the ER specifically to target lysosomal enzymes to lysosomes and prevent their secretion. Lysosomal enzymes in the ER receive an N-linked oligosaccharide, like plasma membrane and secreted proteins, but are then phosphorylated on one or two mannose residues. The phosphorylation of mannose residues occurs in two steps, the first step being the addition of an N-acetylglucosamine phosphate residue by N-acetylglucosamine phosphotransferase, and the second the removal of the N-acetylglucosamine group by phosphodiesterase. The phosphorylated mannose residue then targets the lysosomal enzyme to a mannose 6-phosphate receptor which transports it to a lysosome vesicle (Lodish, supra, pp.708-711).

Chaperones

Molecular chaperones are proteins that aid in the proper folding of immature proteins and refolding of improperly folded ones, the assembly of protein subunits, and in the transport of unfolded proteins across membranes. Chaperones are also called heat-shock proteins (hsp) because of their tendency to be expressed in dramatically increased amounts following brief exposure of cells to elevated temperatures. This latter property most likely reflects their need in the refolding of proteins that have become denatured by the high temperatures. Chaperones may be divided into several classes according to their location, function, and molecular weight, and include hsp60, TCP1, hsp70, hsp40 (also called DnaJ), and hsp90. For example, hsp90 binds to steroid hormone receptors, represses transcription in the absence of the ligand, and provides proper folding of the ligand-binding domain of the receptor in the presence of the hormone (Burston, S. G. and A. R. Clarke (1995) Essays Biochem. 29:125-136). Hsp60 and hsp70 chaperones aid in the transport and folding of newly synthesized proteins. Hsp70 acts early in protein folding, binding a newly synthesized protein before it leaves the ribosome and transporting the protein to the mitochondria or ER before releasing the folded protein. Hsp60, along with hsp10, binds misfolded proteins and gives them the opportunity to refold correctly. All chaperones share an affinity for hydrophobic patches on incompletely folded proteins and the ability to hydrolyze ATP. The energy of ATP hydrolysis is used to release the hsp-bound protein in its properly folded state (Alberts, supra, pp 214, 571-572).

Nucleic Acid Synthesis and Modification Molecules

Polymerases

DNA and RNA replication are critical processes for cell replication and function. DNA and RNA replication are mediated by the enzymes DNA and RNA polymerase, respectively, by a “templating” process in which the nucleotide sequence of a DNA or RNA strand is copied by complementary base-pairing into a complementary nucleic acid sequence of either DNA or RNA. However, there are fundamental differences between the two processes.

DNA polymerase catalyzes the stepwise addition of a deoxyribonucleotide to the 3′-OH end of a polynucleotide strand (the primer strand) that is paired to a second (template) strand. The new DNA strand therefore grows in the 5′ to 3′ direction (Alberts, B. et al. (1994)The Molecular Biology of the Cell, Garland Publishing Inc., New York N.Y., pp. 251-254). The substrates for the polymerization reaction are the corresponding deoxynucleotide triphosphates which must base-pair with the correct nucleotide on the template strand in order to be recognized by the polymerase. Because DNA exists as a double-stranded helix, each of the two strands may serve as a template for the formation of a new complementary strand. Each of the two daughter cells of the dividing cell therefore inherits a new DNA double helix containing one old and one new strand. Thus, DNA is said to be replicated “semiconservatively” by DNA polymerase. In addition to the synthesis of new DNA, DNA polymerase is also involved in the repair of damaged DNA as discussed below under “Ligases.”

In contrast to DNA polymerase, RNA polymerase uses a DNA template strand to “transcribe” DNA into RNA using ribonucleotide triphosphates as substrates. Like DNA polymerization, RNA polymerization proceeds in a 5′ to 3′ direction by addition of a ribonucleoside monophosphate to the 3′-OH end of a growing RNA chain DNA transcription generates messenger RNAs (mRNA) that carry information for protein synthesis, as well as the transfer, ribosomal, and other RNAs that have structural or catalytic functions. In eukaryotes, three discrete RNA polymerases synthesize the three different types of RNA (Alberts, supra, pp. 367-368). RNA polymerase I makes the large ribosomal RNAs, RNA polymerase II makes the mRNAs that will be translated into proteins, and RNA polymerase III makes a variety of small, stable RNAs, including 5S ribosomal RNA and the transfer RNAs (tRNA). In all cases, RNA synthesis is initiated by binding of the RNA polymerase to a promoter region on the DNA and synthesis begins at a start site within the promoter. Synthesis is completed at a broad, general stop or termination region in the DNA where both the polymerase and the completed RNA chain are released.

Ligases

DNA repair is the process by which accidental base changes, such as those produced by oxidative damage, hydrolytic attack, or uncontrolled methylation of DNA are corrected before replication or transcription of the DNA can occur. Because of the efficiency of the DNA repair process, fewer than one in one thousand accidental base changes causes a mutation (Alberts, sutra, pp. 245-249). The three steps common to most types of DNA repair are (1) excision of the damaged or altered base or nucleotide by DNA nucleases, leaving a gap; (2) insertion of the correct nucleotide in this gap by DNA polymerase using the complementary strand as the template; and (3) sealing the break left between the inserted nucleotide(s) and the existing DNA strand by DNA ligase. In the last reaction, DNA ligase uses the energy from ATP hydrolysis to activate the 5′ end of the broken phosphodiester bond before forming the new bond with the 3′-OH of the DNA strand. In Bloom's syndrome, an inherited human disease, individuals are partially deficient in DNA ligation and consequently have an increased incidence of cancer (Alberts, supra, p. 247).

Nucleases

Nucleases comprise both enzymes that hydrolyze DNA (DNase) and RNA (RNase). They serve different purposes in nucleic acid metabolism. Nucleases hydrolyze the phosphodiester bonds between adjacent nucleotides either at internal positions (endonucleases) or at the terminal 3′ or, 5′ nucleotide positions (exonucleases). A DNA exonuclease activity in DNA polymerase, for example, serves to remove improperly paired nucleotides attached to the 3′-OH end of the growing DNA strand by the polymerase and thereby serves a “proofreading” function. As mentioned above, DNA endonuclease activity is involved in the excision step of the DNA repair process.

RNases also serve a variety of functions. For example, RNase P is a ribonucleoprotein enzyme which cleaves the 5′ end of pre-tRNAs as part of their maturation process. RNase H digests the RNA strand of an RNA/DNA hybrid Such hybrids occur in cells invaded by retroviruses, and RNase H is an important enzyme in the retroviral replication cycle. Pancreatic RNase secreted by the pancreas into the intestine hydrolyzes RNA present in ingested foods. RNase activity in serum and cell extracts is elevated in a variety of cancers and infectious diseases (Schein, C. H. (1997) Nat Biotechnol. 15:529-536). Regulation of RNase activity is being investigated as a means to control tumor angiogenesis, allergic reactions, viral infection and replication, and fungal infections.

Methylases

Methylation of specific nucleotides occurs in both DNA and RNA, and serves different functions in the two macromolecules. Methylation of cytosine residues to form 5-methyl cytosine in DNA occurs specifically at CG sequences which are base-paired with one another in the DNA double-helix. This pattern of methylation is passed from generation to generation during DNA replication by an enzyme called “maintenance methylase” that acts preferentially on those CG sequences that are base-paired with a CG sequence that is already methylated. Such methylation appears to distinguish active from inactive genes by preventing the binding of regulatory proteins that “turn on”, the gene, but permit the binding of proteins that inactivate the gene (Alberts, supra, pp. 448-451). In RNA metabolism, “tRNA methylase” produces one of several nucleotide modifications in tRNA that affect the conformation and base-pairing of the molecule and facilitate the recognition of the appropriate mRNA codons by specific tRNAs. The primary methylation pattern is the dimethylation of guanine residues to form N,N-dimethyl guanine.

Helicases and Single-Stranded Binding Proteins

Helicases are enzymes that destabilize and unwind double helix structures in both DNA and RNA. Since DNA replication occurs more or less simultaneously on both strands, the two strands must first separate to generate a replication “fork” for DNA polymerase to act on. Two types of replication proteins contribute to this process, DNA helicases and single-stranded binding proteins. DNA helicases hydrolyze ATP and use the energy of hydrolysis to separate the DNA strands. Single-stranded binding proteins (SSBs) then bind to the exposed DNA strands without covering the bases, thereby temporarily stabilizing them for templating by the DNA polymerase (Alberts, supra, pp. 255-256).

RNA helicases also alter and regulate RNA conformation and secondary structure. Like the DNA helicases, RNA helicases utilize energy derived from ATP hydrolysis to destabilize and unwind RNA duplexes. The most well characterized and ubiquitous family of RNA helicases is the DEAD-box family, so named for the conserved B-type ATP-binding motif which is diagnostic of proteins in this family. Over 40 DEAD-box helicases have been identified in organisms as diverse as bacteria, insects, yeast, amphibians, mammals, and plants. DEAD-box helicases function in diverse processes such as translation initiation, splicing, ribosome assembly, and RNA editing, transport, and stability. Some DEAD-box helicases play tissue- and stage-specific roles in spermatogenesis and embryogenesis. Overexpression of the DEAD-box 1 protein (DDX1) may play a role in the progression of neuroblastoma (Nb) and retinoblastoma (Rb) tumors (Godbout, R. et al. (1998) J. Biol. Chem. 273:21161-21168). These observations suggest that DDX1 may promote or enhance tumor progression by altering the normal secondary structure and expression levels of RNA in cancer cells. Other DEAD-box helicases have been implicated either directly or indirectly in tumorigenesis (Discussed in Godbout, supra). For example, murine p68 is mutated in ultraviolet light-induced tumors, and human DDX6 is located at a chromosomal breakpoint associated with B-cell lymphoma Similarly, a chimeric protein comprised of DDX10 and NUP98, a nucleoporin protein, may be involved in the pathogenesis of certain myeloid malignancies.

Topoisomerases

Besides the need to separate DNA strands prior to replication, the two strands must be “unwound” from one another prior to their separation by DNA helicases. This function is performed by proteins known as DNA topoisomerases. DNA topoisomerase effectively acts as a reversible nuclease that hydrolyzes a phosphodiesterase bond in a DNA strand, permitting the two strands to rotate freely about one another to remove the strain of the helix, and then rejoins the original phosphodiester bond between the two strands. Two types of DNA topoisomerase exist, types I and II. DNA Topoisomerase I causes a single-strand break in a DNA helix to allow the rotation of the two strands of the helix about the remaining phosphodiester bond in the opposite strand DNA topoisomerase II causes a transient break in both strands of a DNA helix where two double helices cross over one another. This type of topoisomerase can efficiently separate two interlocked DNA circles (Alberts, supra, pp.260-262). Type II topoisomerases are largely confined to proliferating cells in eukaryotes, such as cancer cells. For this reason they are targets for anticancer drugs. Topoisomerase II has been implicated in multi-drug resistance (MDR) as it appears to aid in the repair of DNA damage inflicted by DNA binding agents such as doxorubicin and vincristine.

Recombinases

Genetic recombination is the process of rearranging DNA sequences within an organism's genome to provide genetic variation for the organism in response to changes in the environment DNA recombination allows variation in the particular combination of genes present in an individual's genome, as well as the timing and level of expression of these genes (see Alberts, supra, pp. 263-273). Two broad classes of genetic recombination are commonly recognized, general recombination and site-specific recombination. General recombination involves genetic exchange between any homologous pair of DNA sequences usually located on two copies of the same chromosome. The process is aided by enzymes called recombinases that “nick” one strand of a DNA duplex more or less randomly and permit exchange with the complementary strand of another duplex. The process does not normally change the arrangement of genes on a chromosome. In site-specific recombination, the recombinase recognizes specific nucleotide sequences present in one or both of the recombining molecules. Base-pairing is not involved in this form of recombination and therefore does not require DNA homology between the recombining molecules. Unlike general recombination, this form of recombination can alter the relative positions of nucleotide sequences in chromosomes.

Splicing Factors

Various proteins are necessary for processing of transcribed RNAs in the nucleus. Pre-mRNA processing steps include capping at the 5′ end with methylguanosine, polyadenylating the 3′ end, and splicing to remove introns. The primary RNA transcript from DNA is a faithful copy of the gene containing both exon and intron sequences, and the latter sequences must be cut out of the RNA transcript to produce an mRNA that codes for a protein. This “splicing” of the mRNA sequence takes place in the nucleus with the aid of a large, multicomponent ribonucleoprotein complex known as a spliceosome. The spliceosomal complex is composed of five small nuclear ribonucleoprotein particles (snRNPs) designated U1, U2, U4, U5, and U6, and a number of additional proteins. Each snRNP contains a single species of snRNA and about ten proteins. The RNA components of some snRNPs recognize and base pair with intron consensus sequences. The protein components mediate spliceosome assembly and the splicing reaction. Autoantibodies to snRNP proteins are found in the blood of patients with systemic lupus erythematosus (Stryer, L. (1995) Biochemistry, W. H. Freeman and Company, New York N.Y., p. 863).

Adhesion Molecules

The surface of a cell is rich in transmembrane proteoglycans, glycoproteins, glycolipids, and receptors. These macromolecules mediate adhesion with other cells and with components of the extracellular matrix (ECM). The interaction of the cell with its surroundings profoundly influences cell shape, strength, flexibility, motility, and adhesion. These dynamic properties are intimately associated with signal transduction pathways controlling cell proliferation and differentiation, tissue construction, and embryonic development.

Cadherins

Cadherins comprise a family of calcium-dependent glycoproteins that function in mediating cell-cell adhesion in virtually all solid tissues of multicellular organisms. These proteins share multiple repeats of a cadherin-specific motif, and the repeats form the folding units of the cadherin extracellular domain. Cadherin molecules cooperate to form focal contacts, or adhesion plaques, between adjacent epithelial cells. The cadherin family includes the classical cadherins and protocadherins. Classical cadherins include the E-cadherin, N-cadherin, and P-cadherin subfamilies. E-cadherin is present on many types of epithelial cells and is especially important for embryonic development. N-cadherin is present on nerve, muscle, and lens cells and is also critical for embryonic development P-cadherin is present on cells of the placenta and epidermis. Recent studies report that protocadherins are involved in a variety of cell-cell interactions (Suzuki, S. T. (1996) J. Cell Sci. 109:2609-2611). The intracellular anchorage of cadherins is regulated by their dynamic association with catenins, a family of cytoplasmic signal transduction proteins associated with the actin cytoskeleton. The anchorage of cadherins to the actin cytoskeleton appears to be regulated by protein tyrosine phosphorylation, and the cadherins are the target of phosphorylation-induced junctional disassembly (Aberle, H. et al. (1996) J. Cell. Biochem. 61:514-523).

Integrins

Integrins are ubiquitous transmembrane adhesion molecules that link the ECM to the internal cytoskeleton. Integrins are composed of two noncovalently associated transmembrane glycoprotein subunits called α and β. Integrins function as receptors that play a role in signal transduction. For example, binding of integrin to its extracellular ligand may stimulate changes in intracellular calcium levels or protein kinase activity (Sjaastad, M. D. and W. J. Nelson (1997) BioEssays 19:47-55). At least ten cell surface receptors of the integrin family recognize the ECM component fibronectin, which is involved in many different biological processes including cell migration and embryogenesis (Johansson, S. et al. (1997) Front. Biosci. 2:D126-D146).

Lectins

Lectins comprise a ubiquitous family of extracellular glycoproteins which bind cell surface carbohydrates specifically and reversibly, resulting in the agglutination of cells (reviewed in Drickamer, K. and M. E. Taylor (1993) Annu. Rev. Cell Biol. 9:237-264). This function is particularly important for activation of the immune response. Lectins mediate the agglutination and mitogenic stimulation of lymphocytes at sites of inflammation (Lasky, L. A. (1991) J. Cell. Biochem. 45:139-146; Paietta, E. et al. (1989) J. Immunol. 143:2850-2857).

Lectins are further classified into subfamilies based on carbohydrate-binding specificity and other criteria. The galectin subfamily, in particular, includes lectins that bind β-galactoside carbohydrate moieties in a thio]-dependent manner (reviewed in Hadari, Y. R. et al. (1998) J. Biol. Chem. 270:3447-3453). Galectins are widely expressed and developmentally regulated. Because all galectins lack an N-terminal signal peptide, it is suggested that galectins are externalized through an a typical secretory mechanism. Two classes of galectins have been defined based on molecular weight and oligomerization properties. Small galectins form homodimers and are about 14 to 16 kilodaltons in mass, while large galectins are monomeric and about 29-37 kilodaltons.

Galectins contain a characteristic carbohydrate recognition domain (CRD). The CRD is about 140 amino acids and contains several stretches of about 1-10 amino acids which are highly conserved among all galectins. A particular 6-amino acid motif within the CRD contains conserved tryptophan and arginine residues which are critical for carbohydrate binding. The CRD of some galectins also contains cysteine residues which may be important for disulfide bond formation. Secondary structure predictions indicate that the CRD forms several β-sheets.

Galectins play a number of roles in diseases and conditions associated with cell-cell and cell-matrix interactions. For example, certain galectins associate with sites of inflammation and bind to cell surface immunoglobulin E molecules. In addition, galectins may play an important role in cancer metastasis. Galectin overexpression is correlated with the metastatic potential of cancers in humans and mice. Moreover, anti-galectin antibodies inhibit processes associated with cell transformation, such as cell aggregation and anchorage-independent growth (See, for example, Su, Z.-Z. et al. (1996) Proc. Natl. Acad. Sci. USA 93:7252-7257).

Selectins

Selectins, or LEC-CAMs, comprise a specialized lectin subfamily involved primarily in inflammation and leukocyte adhesion (Reviewed in Lasky, supra). Selectins mediate the recruitment of leukocytes from the circulation to sites of acute inflammation and are expressed on the surface of vascular endothelial cells in response to cytokine signaling. Selectins bind to specific ligands on the leukocyte cell membrane and enable the leukocyte to adhere to and migrate along the endothelial surface. Binding of selectin to its ligand leads to polarized rearrangement of the actin cytoskeleton and stimulates signal transduction within the leukocyte (Brenner, B. et al. (1997) Biochem. Biophys. Res. Commun 231:802-807;, Hidari, K. I. et al. (1997) J. Biol. Chem. 272:28750-28756). Members of the selectin family possess three characteristic motifs: a lectin or carbohydrate recognition domain; an epidermal growth factor-like domain; and a variable number of short consensus repeats (scr or “sushi” repeats) which are also present in complement regulatory proteins. The selectins include lymphocyte adhesion molecule-1 (Lam-1 or L-selectin), endothelial leukocyte adhesion molecule-1 (ELAM-1 or E-selectin), and granule membrane protein-140 (GMP-140 or P-selectin) (Johnston, G. I. et al. (1989) Cell 56:1033-1044).

Antigen Recognition Molecules

All vertebrates have developed sophisticated and complex immune systems that provide protection from viral, bacterial, fungal, and parasitic infections. A key feature of the immune system is its ability to distinguish foreign molecules, or antigens, from “self” molecules. This ability is mediated primarily by secreted and transmembrane proteins expressed by leukocytes (white blood cells) such as lymphocytes, granulocytes, and monocytes. Most of these proteins belong to the immunoglobulin (1 g) superfamily, members of which contain one or more repeats of a conserved structural domain. This Ig domain is comprised of antiparallel β sheets joined by a disulfide bond in an arrangement called the Ig fold. Members of the Ig superfamily include T-cell receptors, major histocompatibility (MHC) proteins, antibodies, and immune cell-specific surface markers such as CD4, CD8, and CD28.

MHC proteins are cell surface markers that bind to and present foreign antigens to T cells. MHC molecules are classified as either class I or class II. Class I MHC molecules (MHC I) are expressed on the surface of almost all cells and are involved in the presentation of antigen to cytotoxic T cells. For example, a cell infected with virus will degrade intracellular viral proteins and express the protein fragments bound to MHC I molecules on the cell surface. The MHC 1/antigen complex is recognized by cytotoxic T-cells which destroy the infected cell and the virus within Class II MHC molecules are expressed primarily on specialized antigen-presenting cells of the immune system, such as B-cells and macrophages. These cells ingest foreign proteins from the extracellular fluid and express MHC II/antigen complex on the cell surface. This complex activates helper T-cells, which then secrete cytokines and other factors that stimulate the immune response. MHC molecules also play an important role in organ rejection following transplantation. Rejection occurs when the recipient's T-cells respond to foreign MHC molecules on the transplanted organ in the same way as to self MHC molecules bound to foreign antigen. (Reviewed in Alberts, B. et al. (1994) Molecular Biology of the Cell, Garland Publishing, New York N.Y., pp. 1229-1246.)

Antibodies, or immunoglobulins, are either expressed on the surface of B-cells or secreted by B-cells into the circulation. Antibodies bind and neutralize foreign antigens in the blood and other extracellular fluids. The prototypical antibody is a tetramer consisting of two identical heavy polypeptide chains (H-chains) and two identical light polypeptide chains (L-chains) interlinked by disulfide bonds. This arrangement confers the characteristic Y-shape to antibody molecules. Antibodies are classified based on their H-chain composition. The five antibody classes, IgA, IgD, IgE, IgG and IgM, are defined by the α, δ, ε, γ, and μ H-chain types. There are two types of L-chains, κ and λ, either of which may associate as a pair with any H-chain pair. IgG, the most common class of antibody found in the circulation, is tetrameric, while the other classes of antibodies are generally variants or multimers of this basic structure.

H-chains and L-chains each contain an N-terminal variable region and a C-terminal constant region. The constant region consists of about 110 amino acids in L-chains and about 330 or 440 amino acids in H-chains. The amino acid sequence of the constant region is nearly identical among H- or L-chains of a particular class. The variable region consists of about 110 amino acids in both H- and L-chains. However, the amino acid sequence of the variable region differs among H- or L-chains of a particular class. Within each H- or L-chain variable region are three hypervariable regions of extensive sequence diversity, each consisting of about 5 to 10 amino acids. In the antibody molecule, the H- and L-chain hypervariable regions come together to form the antigen recognition site. (Reviewed in Alberts, supra, pp. 1206-1213 and 1216-1217.)

Both H-chains and L-chains contain repeated Ig domains. For example, a typical H-chain contains four Ig domains, three of which occur within the constant region and one of which occurs within the variable region and contributes to the formation of the antigen recognition site. Likewise, a typical L-chain contains two Ig domains, one of which occurs within the constant region and one of which occurs within the variable region.

The immune system is capable of recognizing and responding to any foreign molecule that enters the body. Therefore, the immune system must be armed with a full repertoire of antibodies against all potential antigens. Such antibody diversity is generated by somatic rearrangement of gene segments encoding variable and constant regions. These gene segments are joined together by site-specific recombination which occurs between highly conserved DNA sequences that flank each gene segment. Because there are hundreds of different gene segments, millions of unique genes can be generated combinatorially. In addition, imprecise joining of these segments and an unusually high rate of somatic mutation within these segments further contribute to the generation of a diverse antibody population.

T-cell receptors are both structurally and functionally related to antibodies. (Reviewed in Alberts, supra, pp. 1228-1229.) T-cell receptors are cell surface proteins that bind foreign antigens and mediate diverse aspects of the immune response. A typical T-cell receptor is a heterodimer comprised of two disulfide-linked polypeptide chains called α and β. Each chain is about 280 amino acids in length and contains one variable region and one constant region. Each variable or constant region folds into an Ig domain. The variable regions from the α and β chains come together in the heterodimer to form the antigen recognition site. Tell receptor diversity is generated by somatic rearrangement of gene segments encoding the α and β chains. TV receptors recognize small peptide antigens that are expressed on the surface of antigen-presenting cells and pathogen-infected cells. These peptide antigens are presented on the cell surface in association with major histocompatibility proteins which provide the proper context for antigen recognition.

Secreted and Extracellular Matrix Molecules

Protein secretion is essential for cellular function. Protein secretion is mediated by a signal peptide located at the amino terminus of the protein to be secreted. The signal peptide is comprised of about ten to twenty hydrophobic amino acids which target the nascent protein from the ribosome to the endoplasmic reticulum (ER). Proteins targeted to the ER may either proceed through the secretory pathway or remain in any of the secretory organelles such as the ER, Golgi apparatus, or lysosomes.

Proteins that transit through the secretory pathway are either secreted into the extracellular space or retained in the plasma membrane. Secreted proteins are often synthesized as inactive precursors that are activated by post-translational processing events during transit through the secretory pathway. Such events include glycosylation, proteolysis, and removal of the signal peptide by a signal peptidase. Other events that may occur during protein transport include chaperone-dependent unfolding and folding of the nascent protein and interaction of the protein with a receptor or pore complex. Examples of secreted proteins with amino terminal signal peptides include receptors, extracellular matrix molecules, cytokines, hormones, growth and differentiation factors, neuropeptides, vasomediators, ion channels, transporters/pumps, and proteases. (Reviewed in Alberts, B. et al. (1994) Molecular Biology of The Cell, Garland Publishing, New York N.Y., pp. 557-560, 582-592.)

The extracellular matrix (EC) is a complex network of glycoproteins, polysaccharides, proteoglycans, and other macromolecules that are secreted from the cell into the extracellular space. The ECM remains in close association with the cell surface and provides a supportive meshwork that profoundly influences cell shape, motility, strength, flexibility, and adhesion. In fact, adhesion of a cell to its surrounding matrix is required for cell survival except in the case of metastatic tumor cells, which have overcome the need for cell-ECM anchorage. This phenomenon suggests that the ECM plays a critical role in the molecular mechanisms of growth control and metastasis. (Reviewed in Ruoslahti, E. (1996) Sci. Am 275:72-77.) Furthermore, the ECM determines the structure and physical properties of connective tissue and is particularly important for morphogenesis and other processes associated with embryonic development and pattern formation.

The collagens comprise a family of ECM proteins that provide structure to bone, teeth, skin, ligaments, tendons, cartilage, blood vessels, and basement membranes. Multiple collagen proteins have been identified. Three collagen molecules fold together in a triple helix stabilized by interchain disulfide bonds. Bundles of these triple helices then associate to form fibrils. Collagen primary structure consists of hundreds of (Gly-X-Y) repeats where about a third of the X and Y residues are Pro. Glycines are crucial to helix formation as the bulkier amino acid sidechains cannot fold into the triple helical conformation. Because of these strict sequence requirements, mutations in collagen genes have severe consequences. Osteogenesis imperfecta patients have brittle bones that fracture easily; in severe cases patients die in utero or at birth Ehlers-Danlos syndrome patients have hyperelastic skin, hypermobile joints, and susceptibility to aortic and intestinal rupture. Chondrodysplasia patients have short stature and ocular disorders. Alport syndrome patients have hematuria, sensorineural deafness, and eye lens deformation. (Isselbacher, K. J. et al. (1994) Harrison's Principles of Internal Medicine, McGraw-Hill, Inc., New York N.Y., pp. 2105-2117; and Creighton, T. E. (1984) Proteins. Structures and Molecular Principles, W. H. Freeman and Company, New York N.Y., pp. 191-197.)

Elastin and related proteins confer elasticity to tissues such as skin, blood vessels, and lungs. Elastin is a highly hydrophobic protein of about 750 amino acids that is rich in proline and glycine residues. Elastin molecules are highly cross-linked, forming an extensive extracellular network of fibers and sheets. Elastin fibers are surrounded by a sheath of microfibrils which are composed of a number of glycoproteins, including fibrillin. Mutations in the gene encoding fibrillin are responsible for Marfan's syndrome, a genetic disorder characterized by defects in connective tissue. In severe cases, the aortas of afflicted individuals are prone to rupture. (Reviewed in Alberts, supra, pp. 984-986.)

Fibronectin is a large ECM glycoprotein found in all vertebrates. Fibronectin exists as a dimer of two subunits, each containing about 2,500 amino acids. Each subunit folds into a rod-like structure containing multiple domains. The domains each contain multiple repeated modules, the most common of which is the type III fibronectin repeat. The type III fibronectin repeat is about 90 amino acids in length and is also found in other ECM proteins and in some plasma membrane and cytoplasmic proteins. Furthermore, some type III fibronectin repeats contain a characteristic tripeptide consisting of Arginine-Glycine-Aspartic acid (RGD). The RGD sequence is recognized by the integrin family of cell surface receptors and is also found in other ECM proteins. Disruption of both copies of the gene encoding fibronectin causes early embryonic lethality in mice. The mutant embryos display extensive morphological defects, including defects in the formation of the notochord, somites, heart, blood vessels, neural tube, and extraembryonic structures. (Reviewed in Alberts, supra, pp. 986-987.)

Laminin is a major glycoprotein component of the basal lamina which underlies and supports epithelial cell sheets. Laminin is one of the first ECM proteins synthesized in the developing embryo. Laminin is an 850 kilodalton protein composed of three polypeptide chains joined in the shape of a cross by disulfide bonds. Laminin is especially important for angiogenesis and in particular, for guiding the formation of capillaries. (Reviewed in Alberts, supra, pp. 990-991.)

There are many other types of proteinaceous ECM components, most of which can be classified as proteoglycans. Proteoglycans are composed of unbranched polysaccharide chains (glycosaminoglycans) attached to protein cores. Common proteoglycans include aggrecan, betaglycan, decorin, perlecan, serglycin, and syndecan-1. Some of these molecules not only provide mechanical support, but also bind to extracellular signaling molecules, such as fibroblast growth factor and transforming growth factor β, suggesting a role for proteoglycans in cell-cell communication and cell growth (Reviewed in Alberts, supra, pp. 973-978.) Likewise, the glycoproteins tenascin-C and tenascin-R are expressed in developing and lesioned neural tissue and provide stimulatory and anti-adhesive (inhibitory) properties, respectively, for axonal growth (Faissner, A (1997) Cell Tissue Res. 290:331-341.)

Cytoskeletal Molecules

The cytoskeleton is a cytoplasmic network of protein fibers that mediate cell shape, structure, and movement. The cytoskeleton supports the cell membrane and forms tracks along which organelles and other elements move in the cytosol. The cytoskeleton is a dynamic structure that allows cells to adopt various shapes and to carry out directed movements. Major cytoskeletal fibers include the microtubules, the microfilaments, and the intermediate filaments. Motor proteins, including myosin, dynein, and kinesin, drive movement of or along the fibers. The motor protein dynamin drives the formation of membrane vesicles. Accessory or associated proteins modify the structure or activity of the fibers while cytoskeletal membrane anchors connect the fibers to the cell membrane.

Tubulins

Microtubules, cytoskeletal fibers with a diameter of about 24 nm, have multiple roles in the cell. Bundles of microtubules form cilia and flagella, which are whip-like extensions of the cell membrane that are necessary for sweeping materials across an epithelium and for swimming of sperm, respectively. Marginal bands of microtubules in red blood cells and platelets are important for these cells' pliability. Organelles, membrane vesicles, and proteins are transported in the cell along tracks of microtubules. For example, microtubules run through nerve cell axons, allowing bi-directional transport of materials and membrane vesicles between the cell body and the nerve terminal. Failure to supply the nerve terminal with these vesicles blocks the transmission of neural signals. Microtubules are also critical to chromosomal movement during cell division. Both stable and short-lived populations of microtubules exist in the cell.

Microtubules are polymers of GTP-binding tubulin protein subunits. Each subunit is a heterodimer of α- and β-tubulin, multiple isoforms of which exist The hydrolysis of GTP is linked to the addition of tubulin subunits at the end of a microtubule. The subunits interact head to tail to form protofilaments; the protofilaments interact side to side to form a microtubule. A microtubule is polarized, one end ringed with α-tubulin and the other with β-tubulin, and the two ends differ in their rates of assembly. Generally, each microtubule is composed of 13 protofilaments although 11 or 15 protofilament-microtubules are sometimes found. Cilia and flagella contain doublet microtubules. Microtubules grow from specialized structures known as centrosomes or microtubule-organizing centers (MTOCs). MTOCs may contain one or two centrioles, which are pinwheel arrays of triplet microtubules. The basal body, the organizing center located at the base of a cilium or flagellum, contains one centriole. Gamma tubulin present in the MTOC is important for nucleating the polymerization of α- and β-tubulin heterodimers but does not polymerize into microtubules.

Microtubule-Associated Proteins

Microtubule-associated proteins (MAPs) have roles in the assembly and stabilization of microtubules. One major family of MAPs, assembly MAPs, can be identified in neurons as well as non-neuronal cells. Assembly MAPs are responsible for cross-linking microtubules in the cytosol These MAPs are organized into two domains: a basic microtubule-binding domain and an acidic projection domain. The projection domain is the binding site for membranes, intermediate filaments, or other microtubules. Based on sequence analysis, assembly MAPs can be further grouped into two types: Type I and Type II. Type I MAPs, which include MAP1A and MAP1B, are large, filamentous molecules that co-purify with microtubules and are abundantly expressed in brain and testes. Type I MAPs contain several repeats of a positively-charged amino acid sequence motif that binds and neutralizes negatively charged tubulin, leading to stabilization of microtubules. MAP1A and MAP1B are each derived from a single precursor polypeptide that is subsequently proteolytically processed to generate one heavy chain and one light chain

Another light chain, LC3, is a 16.4 kDa molecule that binds MAP1A, MAP1B, and microtubules. It is suggested that LC3 is synthesized from a source other than the MAP1A or MAP1B transcripts, and that the expression of LC3 may be important in regulating the microtubule binding activity of MAP1A and MAP1B during cell proliferation (Mann, S. S. et al. (1994) J. Biol. Chem. 269:11492-11497).

Type II MAPs, which include MAP2a, MAP2b, MAP2c, MAP4, and Tau, are characterized by three to four copies of an 18-residue sequence in the microtubule-binding domain MAP2a, MAP2b, and MAP2c are found only in dendrites, MAP4 is found in non-neuronal cells, and Tau is found in axons and dendrites of nerve cells. Alternative splicing of the Tau mRNA leads to the existence of multiple forms of Tau protein. Tau phosphorylation is altered in neurodegenerative disorders such as Alzheimer's disease, Pick's disease, progressive supranuclear palsy, corticobasal degeneration, and familial frontotemporal dementia and Parkinsonism linked to chromosome 17. The altered Tau phosphorylation leads to a collapse of the microtubule network and the formation of intraneuronal Tau aggregates (Spillantini, M. G. and M. Goedert (1998) Trends Neurosci. 21:428-433).

The protein pericentrin is found in the MTOC and has a role in microtubule assembly.

Actins

Microfilaments, cytoskeletal filaments with a diameter of about 7-9 nm, are vital to cell locomotion, cell shape, cell adhesion, cell division, and muscle contraction. Assembly and disassembly of the microfilaments allow cells to change their morphology. Microfilaments are the polymerized form of actin, the most abundant intracellular protein in the eukaryotic cell. Human cells contain six isoforms of actin. The three α-actins are found in different kinds of muscle, nonmuscle, β-actin and nonmuscle γ-actin are found in nonmuscle cells, and another y-actin is found in intestinal smooth muscle cells. G-actin, the monomeric form of actin, polymerizes into polarized, helical F-actin filaments, accompanied by the hydrolysis of ATP to ADP. Actin filaments associate to form bundles and networks, providing a framework to support the plasma membrane and determine cell shape. These bundles and networks are connected to the cell membrane. In muscle cells, thin filaments containing actin slide past thick filaments containing the motor protein myosin during contraction. A family of actin-related proteins exist that are not part of the actin cytoskeleton, but rather associate with microtubules and dynein.

Actin-Associated Proteins

Actin-associated proteins have roles in cross-linking, severing, and stabilization of actin filaments and in sequestering actin monomers. Several of the actin-associated proteins have multiple functions. Bundles and networks of actin filaments are held together by actin cross-lining proteins. These proteins have two actin-binding sites, one for each filament. Short cross-lining proteins promote bundle formation while longer, more flexible cross-linking proteins promote network formation. Calmodulin-like calcium-binding domains in actin cross-linking proteins allow calcium regulation of cross-linking. Group I cross-linking proteins have unique actin-binding domains and include the 30 kD protein, EF-1a, fascin, and scruin. Group II cross-lining proteins have a 7,000-MW actin-binding domain and include villin and dematin. Group III cross-lining proteins have pairs of a 26,000-MW actin-binding domain and include fimbrin, spectrin, dystrophin, ABP 120, and filamin

Severing proteins regulate the length of actin filaments by breaking them into short pieces or by blocking their ends. Severing proteins include gCAP39, severin (fragmin), gelsolin, and villin. Capping proteins can cap the ends of actin filaments, but cannot break filaments. Capping proteins include CapZ and tropomodulin. The proteins thymosin and profilin sequester actin monomers in the cytosol, allowing a pool of unpolymerized actin to exist. The actin-associated proteins tropomyosin, troponin, and caldesmon regulate muscle contraction in response to calcium.

Intermediate Filaments and Associated Proteins

Intermediate filaments (IFs) are cytoskeletal fibers with a diameter of about 10 nm, intermediate between that of microfilaments and microtubules. IFs serve structural roles in the cell, reinforcing cells and organizing cells into tissues. IFs are particularly abundant in epidermal cells and in neurons. IFs are extremely stable, and, in contrast to microfilaments and microtubules, do not function in cell motility.

Five types of IF proteins are known in mammals. Type I and Type II proteins are the acidic and basic keratins, respectively. Heterodimers of the acidic and basic keratins are the building blocks of keratin IFs. Keratins are abundant in soft epithelia such as skin and cornea, hard epithelia such as nails and hair, and in epithelia that line internal body cavities. Mutations in keratin genes lead to epithelial diseases including epidermolysis bullosa simplex, bullous congenital ichthyosiform erytroderma (epidermolytic hyperkeratosis), non-epidermolytic and epidermolytic palmoplantar keratodenna, ichthyosis bullosa of Siemens, pachyonychia congenita, and white sponge nevus. Some of these diseases result in severe skin blistering. (See, e.g., Wawersik, M. et al. (1997) J. Biol. Chem. 272:32557-32565; and Corden L. D. and W. H. McLean (1996) Exp. Dermatol. 5:297-307.)

Type III IF proteins include desmin, glial fibrillary acidic protein, vimentin, and peipherin. Desmin filaments in muscle cells link myofibrils into bundles and stabilize sarcomeres in contracting muscle. Glial fibrillary acidic protein filaments are found in the glial cells that surround neurons and astrocytes. Vimentin filaments are found in blood vessel endothelial cells, some epithelial cells, and mesenchymal cells such as fibroblasts, and are commonly associated with microtubules. Vimentin filaments may have roles in keeping the nucleus and other organelles in place in the cell. Type IV IFs include the neurofilaments and nestin. Neurofilaments, composed of three polypeptides NF-L, NF-M, and NF-H, are frequently associated with microtubules in axons. Neurofilaments are responsible for the radial growth and diameter of an axon, and ultimately for the speed of nerve impulse transmission. Changes in phosphorylation and metabolism of neurofilaments are observed in neurodegenerative diseases including amyotrophic lateral sclerosis, Parkinson's disease, and Alzheimer's disease (Julien, J. P. and W. E. Mushynski (1998) Prog. Nucleic Acid Res. Mol. Biol. 61:1-23). Type V IFs, the lamins, are found in the nucleus where they support the nuclear membrane.

IFs have a central α-helical rod region interrupted by short nonhelical linker segments. The rod region is bracketed, in most cases, by non-helical head and tail domains. The rod regions of intermediate filament proteins associate to form a coiled-coil dimer. A highly ordered assembly process leads from the dimers to the IFs. Neither ATP nor GTP is needed for IF assembly, unlike that of microfilaments and microtubules.

IF-associated proteins (IFAPs) mediate the interactions of IFs with one another and with other cell structures. IFAPs cross-link IFs into a bundle, into a network, or to the plasma membrane, and may cross-link IFs to the microfilament and microtubule cytoskeleton. Microtubules and IFs are in particular closely associated. IFAPs include BPAG1, plakoglobin, desmoplakin I, desmoplakin II, plectin, ankyrin, filaggrin, and lamin B receptor.

Cytoskeletal-Membrane Anchors

Cytoskeletal fibers are attached to the plasma membrane by specific proteins. These attachments are important for maintaining cell shape and for muscle contraction. In erythrocytes, the spectrin-actin cytoskeleton is attached to cell membrane by three proteins, band 4.1, ankyrin, and adducin. Defects in this attachment result in abnormally shaped cells which are more rapidly degraded by the spleen, leading to anemia. In platelets, the spectrin-actin cytoskeleton is also linked to the membrane by ankyrin; a second actin network is anchored to the membrane by filamin In muscle cells the protein dystrophin links actin filaments to the plasma membrane; mutations in the dystrophin gene lead to Duchenne muscular dystrophy. In adherens junctions and adhesion plaques the peripheral membrane proteins α-actin and vinculin attach actin filaments to the cell membrane.

IFs are also attached to membranes by cytoskeletal-membrane anchors. The nuclear lamina is attached to the inner surface of the nuclear membrane by the lamin B receptor. Vimentin IFs are attached to the plasma membrane by ankyrin and plectin. Desmosome and hemidesmosome membrane junctions hold together epithelial cells of organs and skin. These membrane junctions allow shear forces to be distributed across the entire epithelial cell layer, thus providing strength and rigidity to the epithelium. IFs in epithelial cells are attached to the desmosome by plakoglobin and desmoplakins. The proteins that link IFs to hemidesmosomes are not known. Desmin IFs surround the sarcomere in muscle and are linked to the plasma membrane by paranemin, synemin, and ankyrin.

Myosin-related Motor Proteins

Myosins are actin-activated ATPases, found in eukaryotic cells, that couple hydrolysis of ATP with motion. Myosin provides the motor function for muscle contraction and intracellular movements such as phagocytosis and rearrangement of cell contents during mitotic cell division (cytokinesis). The contractile unit of skeletal muscle, termed the sarcomere, consists of highly ordered arrays of thin actin-containing filaments and thick myosin-containing filaments. Crossbridges form between the thick and thin filaments, and the ATP-dependent movement of myosin heads within the thick filaments pulls the thin filaments, shortening the sarcomere and thus the muscle fiber.

Myosins are composed of one or two heavy chains and associated light chains. Myosin heavy chains contain an amino-terminal motor or head domain, a neck that is the site of light-chain binding, and a carboxy-terminal tail domain. The tail domains may associate to form an α-helical coiled coil. Conventional myosins, such as those found in muscle tissue, are composed of two myosin heavy-chain subunits, each associated with two light-chain subunits that bind at the neck region and play a regulatory role. Unconventional myosins, believed to function in intracellular motion, may contain either one or two heavy chains and associated light chains. There is evidence for about 25 myosin heavy chain genes in vertebrates, more than half of them unconventional.

Dynein-related Motor Proteins

Dyneins are (−) end-directed motor proteins which act on microtubules. Two classes of dyneins, cytosolic and axonemal, have been identified. Cytosolic dyneins are responsible for translocation of materials along cytoplasmic microtubules, for example, transport from the nerve terminal to the cell body and transport of endocytic vesicles to lysosomes. Cytoplasmic dyneins are also reported to play a role in mitosis. Axonemal dyneins are responsible for the beating of flagella and cilia. Dynein on one microtubule doublet walks along the adjacent microtubule doublet This sliding force produces bending forces that cause the flagellum or cilium to beat Dyneins have a native mass between 1000 and 2000 kDa and contain either two or three force-producing heads driven by the hydrolysis of ATP. The heads are linked via stalks to a basal domain which is composed of a highly variable number of accessory intermediate and light chains.

Kinesin-related Motor Proteins

Kinesins are (+) end-directed motor proteins which act on microtubules. The prototypical kinesin molecule is involved in the transport of membrane-bound vesicles and organelles. This function is particularly important for axonal transport in neurons. Kinesin is also important in all cell types for the transport of vesicles from the Golgi complex to the endoplasmic reticulum. This role is critical for maintaining the identity and functionality of these secretory organelles.

Kinesins define a ubiquitous, conserved family of over 50 proteins that can be classified into at least 8 subfamilies based on primary amino acid sequence, domain structure, velocity of movement, and cellular function. (Reviewed in Moore, J. D. and S. A. Endow (1996) Bioessays 18:207-219; and Hoyt, A. M. (1994) Curr. Opin. Cell Biol. 6:63-68.) The prototypical kinesin molecule is a heterotetramer comprised of two heavy polypeptide chains (KHCs) and two light polypeptide chains (KLCs). The KHC subunits are typically referred to as “kinesin.” KHC is about 1000 amino acids in length, and KLC is about 550 amino acids in length Two KHCs dimerize to form a rod-shaped molecule with three distinct regions of secondary structure. At one end of the molecule is a globular motor domain that functions in ATP hydrolysis and microtubule binding. Kinesin motor domains are highly conserved and share over 70% identity. Beyond the motor domain is an α-helical coiled-coil region which mediates dimerization. At the other end of the molecule is a fan-shaped tail that associates with molecular cargo. The tail is formed by the interaction of the KHC C-termini with the two KLCs.

Members of the more divergent subfamilies of kinesins are called kinesin-related proteins (KRPs), many of which function during mitosis in eukaryotes (Hoyt, supra). Some KRPs are required for assembly of the mitotic spindle. In vivo and in vitro analyses suggest that these KRPs exert force on microtubules that comprise the mitotic spindle, resulting in the separation of spindle poles. Phosphorylation of KRP is required for this activity. Failure to assemble the mitotic spindle results in abortive mitosis and chromosomal aneuploidy, the latter condition being characteristic of cancer cells. In addition, a unique KRP, centromere protein E, localizes to the kinetochore of human mitotic chromosomes and may play a role in their segregation to opposite spindle poles.

Dynamin-related Motor Proteins

Dynamin is a large GTPase motor protein that functions as a “molecular pinchase,” generating a mechanochemical force used to sever membranes. This activity is important in forming clathrin-coated vesicles from coated pits in endocytosis and in the biogenesis of synaptic vesicles in neurons. Binding of dynamin to a membrane leads to dynamin's self-assembly into spirals that may act to constrict a flat membrane surface into a tubule. GTP hydrolysis induces a change in conformation of the dynamin polymer that pinches the membrane tubule, leading to severing of the membrane tubule and formation of a membrane vesicle. Release of GDP and inorganic phosphate leads to dynamin disassembly. Following disassembly the dynamin may either dissociate from the membrane or remain associated to the vesicle and be transported to another region of the cell. Three homologous dynamin genes have been discovered, in addition to several dynamin-related proteins. Conserved dynamin regions are the N-terminal GTP-binding domain, a central pleckstrin homology domain that binds membranes, a central coiled-coil region that may activate dynamin's GTPase activity, and a C-terminal proline-rich domain that contains several motifs that bind SH3 domains on other proteins. Some dynamin-related proteins do not contain the pleckstrin homology domain or the proline-rich domain. (See McNiven, M. A. (1998) Cell 94:151-154; Scaife, R. N. and R. L. Margolis (1997) Cell. Signal. 9:395401.)

The cytoskeleton is reviewed in Lodish, H. et al. (1995) Molecular Cell Biology, Scientific American Books, New York N.Y.

Ribosomal Molecules

Ribosomal RNAs (rRNAs) are assembled, along with ribosomal proteins, into ribosomes, which are cytoplasmic particles that translate messenger RNA into polypeptides. The eukaryotic ribosome is composed of a 60S (large) subunit and a 40S (small) subunit, which together form the 80S ribosome. In addition to the 18S, 28S, 5S, and 5.8S rRNAs, the ribosome also contains more than fifty proteins. The ribosomal proteins have a prefix which denotes the subunit to which they belong, either L (large) or S (small). Ribosomal protein activities include binding rRNA and organizing the conformation of the junctions between rRNA helices (Woodson, S. A. and N. B. Leontis (1998) Curr. Opin. Stuct. Biol. 8:294-300; Ramakrishnan, V. and S. W. White (1998) Trends Biochem. Sci. 23:208-212.) Three important sites are identified on the ribosome. The aminoacyl-tRNA site (A site) is where charged tRNAs (with the exception of the initiator-tRNA) bind on arrival at the ribosome. The peptidyl-tRNA site (P site) is where new peptide bonds are formed, as well as where the initiator tRNA binds. The exit site E site) is where deacylated tRNAs bind prior to their release from the ribosome. (The ribosome is reviewed in Stryer, L. (1995) Biochemistry W. H. Freeman and Company, New York N. Y., pp. 888-908; and Lodish, H. et al. (1995) Molecular Cell Biology Scientific American Books, New York N.Y. pp. 119-138.)

Chromatin Molecules

The nuclear DNA of eukaryotes is organized into chromatin. Two types of chromatin are observed: euchromatin, some of which may be transcribed, and heterochromatin so densely packed that much of it is inaccessible to transcription. Chromatin packing thus serves to regulate protein expression in eukaryotes. Bacteria lack chromatin and the chromatin-packing level of gene regulation. The fundamental unit of chromatin is the nucleosome of 200 DNA base pairs associated with two copies each of histones H2A, H2B, H3, and H4. Adjascent nucleosomes are linked by another class of histones, H1. Low molecular weight non-histone proteins called the high mobility group (ERG), associated with chromatin, may function in the unwinding of DNA and stabilization of single-stranded DNA. Chromodomain proteins function in compaction of chromatin into its transcriptionally silent heterochromatin form.

During mitosis, all DNA is compacted into heterochromatin and transcription ceases. Transcription in interphase begins with the activation of a region of chromatin Active chromatin is decondensed. Decondensation appears to be accompanied by changes in binding coefficient, phosphorylation and acetylation states of chromatin histones. HMG proteins HMG13 and HMG17 selectively bind activated chromatin. Topoisomerases remove superheilcal tension on DNA The activated region decondenses, allowing gene regulatory proteins and transcription factors to assemble on the DNA.

Patterns of chromatin structure can be stably inherited, producing heritable patterns of gene expression. In mammals, one of the two X chromosomes in each female cell is inactivated by condensation to heterochromatin during zygote development. The inactive state of this chromosome is inherited, so that adult females are mosaics of clusters of paternal-X and maternal-X clonal cell groups. The condensed X chromosome is reactivated in meiosis.

Chromatin is associated with disorders of protein expression such as thalassemia, a genetic anemia resulting from the removal of the locus control region (LCR) required for decondensation of the globin gene locus.

For a review of chromatin structure and function see Alberts, B. et al. (1994) Molecular Cell Biology, third edition, Garland Publishing, Inc., New York N.Y., pp. 351-354, 433-439.

Electron Transfer Associated Molecules

Electron carriers such as cytochromes accept electrons from NADH or FADH₂ and donate them to other electron carriers. Most electron-transferring proteins, except ubiquinone, are prosthetic groups such as flavins, heme, FeS clusters, and copper, bound to inner membrane proteins. Adrenodoxin, for example, is an FeS protein that forms a complex with NADPH:adrenodoxin reductase and cytochrome p450. Cytochromes contain a heme prosthetic group, a porphyrin ring containing a tightly bound iron atom. Electron transfer reactions play a crucial role in cellular energy production.

Energy is produced by the oxidation of glucose and fatty acids. Glucose is initially converted to pyruvate in the cytoplasm. Fatty acids and pyruvate are transported to the mitochondria for complete oxidation to CO₂ coupled by enzymes to the transport of electrons from NADH and FADH₂ to oxygen and to the synthesis of ATP (oxidative phosphorylation) from ADP and Pᵢ.

Pyruvate is transported into the mitochondria and converted to acetyl-CoA for oxidation via the citric acid cycle, involving pyruvate dehydrogenase components, dihydrolipoyl transacetylase, and dihydrolipoyl dehydrogenase. Enzymes involved in the citric acid cycle include: citrate synthetase, aconitases, isocitrate dehydrogenase, alpha-ketoglutarate dehydrogenase complex including transsuccinylases, succinyl CoA synthetase, succinate dehydrogenase, fumarases, and malate dehydrogenase. Acetyl CoA is oxidized to CO₂ with concomitant formation of NADH, FADH₂, and GTP. In oxidative phosphorylation, the transfer of electrons from NADH and FADH₂ to oxygen by dehydrogenases is coupled to the synthesis of ATP from ADP and Pᵢ by the F₀F₁ ATPase complex in is the mitochondrial inner membrane. Enzyme complexes responsible for electron transport and ATP synthesis include the F₀F₁ ATPase complex, ubiquinone(CoQ)-cytocbrome c reductase, ubiquinone reductase, cytochrome b, cytocbrome c₁, FeS protein, and cytochrome c oxidase.

ATP synthesis requires membrane transport enzymes including the phosphate transporter and the ATP-ADP antiport protein. The ATP-binding casette (ABC) superfamily has also been suggested as belonging to the mitochondrial transport group (Hogue, D. L. et al. (1999) J. Mol. Biol. 285:379-389). Brown fat uncoupling protein dissipates oxidative energy as heat, and may be involved the fever response to infection and trauma (Cannon, B. et al. (1998) Ann. NY Acad. Sci. 856:171-187).

Mitochondria are oval-shaped organelles comprising an outer membrane, a tightly folded inner membrane, an intermembrane space between the outer and inner membranes, and a matrix inside the inner membrane. The outer membrane contains many porin molecules that allow ions and charged molecules to enter the intermembrane space, while the inner membrane contains a variety of transport proteins that transfer only selected molecules. Mitochondria are the primary sites of energy production in cells.

Mitochondria contain a small amount of DNA. Human mitochondrial DNA encodes 13 proteins, 22 tRNAs, and 2 rRNAs. Mitochondrial-DNA encoded proteins include NADH-Q reductase, a cytochrome reductase subunit, cytochrome oxidase subunits, and ATP synthase subunits.

Electron-transfer reactions also occur outside the mitochondria in locations such as the endoplasmic reticulum, which plays a crucial role in lipid and protein biosynthesis. Cytochrome b5 is a central electron donor for various reductive reactions occurring on the cytoplasmic surface of liver endoplasmic reticulum. Cytochrome b5 has been found in Golgi, plasma, endoplasmic reticulum (ER), and microbody membranes.

For a review of mitochondrial metabolism and regulation, see Lodish, H. et al. (1995) Molecular Cell Biology, Scientific American Books, New York N.Y., pp. 745-797 and Stryer (1995) Biochemistry, W. H. Freeman and Co., San Francisco Calif., pp 529-558, 988-989.

The majority of mitochondrial proteins are encoded by nuclear genes, are synthesized on cytosolic ribosomes, and are imported into the mitochondria. Nuclear-encoded proteins which are destined for the mitochondrial matrix typically contain positively-charged amino terminal signal sequences. Import of these preproteins from the cytoplasm requires a multisubunit protein complex in the outer membrane known as the translocase of outer mitochondrial membrane (TOM; previously designated MOM; Pfanner, N. et al. (1996) Trends Biochem. Sci. 21:51-52) and at least three inner membrane proteins which comprise the translocase of inner mitochondrial membrane (TIM; previously designated MIM; Pfanner, supra). An inside-negative membrane potential across the inner mitochondrial membrane is also required for preprotein import. Preproteins are recognized by surface receptor components of the TOM complex and are translocated through a proteinaceous pore formed by other TOM components. Proteins targeted to the matrix are then recognized by the import machinery of the TIM complex. The import systems of the outer and inner membranes can function independently (Segui-Real, B. et al. (1993) EMBO J. 12:2211-2218).

Once precursor proteins are in the mitochondria, the leader peptide is cleaved by a signal peptidase to generate the mature protein. Most leader peptides are removed in a one step process by a protease termed mitochondrial processing peptidase (MPP) (Paces, V. et al. (1993) Proc. Natl. Acad. Sci. USA 90:5355-5358). In some cases a two-step process occurs in which MPP generates an intermediate precursor form which is cleaved by a second enzyme, mitochondrial intermediate peptidase, to generate the mature protein.

Mitochondrial dysfunction leads to impaired calcium buffering, generation of free radicals that may participate in deleterious intracellular and extracellular processes, changes in mitochondrial permeability and oxidative damage which is observed in several neurodegenerative diseases. Neurodegenerative diseases linked to mitochondrial dysfunction include some forms of Alzheimer's disease, Friedreich's ataxia, familial amyotrophic lateral sclerosis, and Huntington's disease (Beal, M. F. (1998) Biochim. Biophys. Acta 1366:211-213). The myocardium is heavily dependent on oxidative metabolism, so mitochondrial dysfunction often leads to heart disease (DiMauro, S. and M. Hirano (1998) Curr. Opin Cardiol 13:190-197). Mitochondria are implicated in disorders of cell proliferation, since they play an important role in a cell's decision to proliferate or self-destruct through apoptosis. The oncoprotein Bcl-2, for example, promotes cell proliferation by stabilizing mitochondrial membranes so that apoptosis signals are not released (Susin, S. A. (1998) Biochim. Biophys. Acta 1366:151-165).

Transcription Factor Molecules

Multicellular organisms are comprised of diverse cell types that differ dramatically both in structure and function. The identity of a cell is determined by its characteristic pattern of gene expression, and different cell types express overlapping but distinctive sets of genes throughout development. Spatial and temporal regulation of gene expression is critical for the control of cell proliferation, cell differentiation, apoptosis, and other processes that contribute to organismal development. Futhermore, gene expression is regulated in response to extracellular signals that mediate cell-cell communication and coordinate the activities of different cell types. Appropriate gene regulation also ensures that cells function efficiently by expressing only those genes whose functions are required at a given time.

Transcriptional regulatory proteins are essential for the control of gene expression. Some of these proteins function as transcription factors that initiate, activate, repress, or terminate gene transcription. Transcription factors generally bind to the promoter, enhancer, and upstream regulatory regions of a gene in a sequence-specific manner, although some factors bind regulatory elements with or downstream of a gene's coding region. Transcription factors may bind to a specific region of DNA singly or as a complex with other accessory factors. (Reviewed in Lewin, B. (1990) Genes IV, Oxford University Press, New York N.Y., and Cell Press, Cambridge Mass., pp. 554-570.)

The double helix structure and repeated sequences of DNA create topological and chemical features which can be recognized by transcription factors. These features are hydrogen bond donor and acceptor groups, hydrophobic patches, major and minor grooves, and regular, repeated stretches of sequence which induce distinct bends in the helix. Typically, transcription factors recognize specific DNA sequence motifs of about 20 nucleotides in length. Multiple, adjacent transcription factor-binding motifs may be required for gene regulation.

Many transcription factors incorporate DNA-binding structural motifs which comprise either α helices or β sheets that bind to the major groove of DNA. Four well-characterized structural motifs are helix-turn-helix, zinc finger, leucine zipper, and helix-loop-helix. Proteins containing these motifs may act alone as monomers, or they may form homo- or heterodimers that interact with DNA.

The helix-turn-helix motif consists of two a helices connected at a fixed angle by a short chain of amino acids. One of the helices binds to the major groove. Helix-turn-helix motifs are exemplified by the homeobox motif which is present in homeodomain proteins. These proteins are critical for specifying the anterior-posterior body axis during development and are conserved throughout the animal kingdom. The Antennapedia and Ultrabithorax proteins of Drosophila melanogaster are prototypical homeodomain proteins (Pabo, C. O. and R. T. Sauer (1992) Annu. Rev. Biochem. 61:1053-1095).

The zinc finger motif, which binds zinc ions, generally contains tandem repeats of about 30 amino acids consisting of periodically spaced cysteine and histidine residues. Examples of this sequence pattern, designated C2H2 and C3HC4 (“RING” finger), have been described Lewin, supra). Zinc finger proteins each contain an α helix and an antiparallel β sheet whose proximity and conformation are maintained by the zinc ion. Contact with DNA is made by the arginine prece ding the α helix and by the second, third, and sixth residues of the α helix. Variants of the zinc finger motif include poorly defined cysteine-rich motifs which bind zinc or other metal ions. These motifs may not contain histidine residues and are generally nonrepetitive.

The leucine zipper motif comprises a stretch of amino acids rich in leucine which can form an amphipathic α helix. This structure provides the basis for dimerization of two leucine zipper proteins. The region adjacent to the leucine zipper is usually basic, and upon protein dimerization, is optimally positioned for binding to the major groove. Proteins containing such motifs are generally referred to as bZIP transcription factors.

The helix-loop-helix motif (HLH) consists of a short α helix connected by a loop to a longer α helix. The loop is flexible and allows the two helices to fold back against each other and to bind to DNA. The transcription factor Myc contains a prototypical HLH motif.

Most transcription factors contain characteristic DNA binding motifs, and variations on the above motifs and new motifs have been and are currently being characterized (Faisst, S. and S. Meyer (1992) Nucleic Acids Res. 20:3-26).

Many neoplastic disorders in humans can be attributed to inappropriate gene expression. Malignant cell growth may result from either excessive expression of tumor promoting genes or insufficient expression of tumor suppressor genes (Cleary, M. L. (1992) Cancer Surv. 15:89-104). Chromosomal translocations may also produce chimeric loci which fuse the coding sequence of one gene with the regulatory regions of a second unrelated gene. Such an arrangement likely results in inappropriate gene transcription, potentially contributing to malignancy.

In addition, the immune system responds to infection or trauma by activating a cascade of events that coordinate the progressive selection, amplification, and mobilization of cellular defense mechanisms. A complex and balanced program of gene activation and repression is involved in this process. However, hyperactivity of the immune system as a result of improper or insufficient regulation of gene expression may result in considerable tissue or organ damage. This damage is well documented in immunological responses associated with arthritis, allergens, heart attack, stroke, and infections (Isselbacher, K. J. et al. (1996) Harrison's Principles of Internal Medicine, 13/e, McGraw Hill, Inc. and Teton Data Systems Software).

Furthermore, the generation of multicellular organisms is based upon the induction and coordination of cell differentiation at the appropriate stages of development Central to this process is differential gene expression, which confers the distinct identities of cells and tissues throughout the body. Failure to regulate gene expression during development can result in developmental disorders. Human developmental disorders caused by mutations in zinc finger-type transcriptional regulators include: urogenenital developmental abnormalities associated with WT1; Greig cephalopolysyndactyly, Pallister-Hall syndrome, and postaxial polydactyly type A (GLI3); and Townes-Brocks syndrome, characterized by anal, renal, limb, and ear abnormalities (SALL1) (Engelkamp, D. and V. van Heyningen (1996) Curr. Opin. Genet Dev. 6:334342; Kohlhase, J. et al. (1999) Am. J. Hum. Genet 64:435445).

Cell Membrane Molecules

Eukaryotic cells are surrounded by plasma membranes which enclose the cell and maintain an environment inside the cell that is distinct from its surroundings. In addition, eukaryotic organisms are distinct from prokaryotes in possessing many intracellular organelle and vesicle structures. Many of the metabolic reactions which distinguish eukaryotic biochemistry from prokaryotic biochemistry take place within these structures. The plasma membrane and the membranes surrounding organelles and vesicles are composed of phosphoglycerides, fatty acids, cholesterol, phospholipids, glycolipids, proteoglycans, and proteins. These components confer identity and functionality to the membranes with which they associate.

Integral Membrane Proteins

The majority of known integral membrane proteins are transmembrane proteins (TM) which are characterized by an extracellular, a transmembrane, and an intracellular domain. TM domains are typically comprised of 15 to 25 hydrophobic amino acids which are predicted to adopt an α-helical conformation. TM proteins are classified as bitopic (Types I and I) and polytopic (Types III and IV) (Singer, S. J. (1990) Annu. Rev. Cell Biol. 6:247-296). Bitopic proteins span the membrane once while polytopic proteins contain multiple membrane-spanning segments. TM proteins function as cell-surface receptors, receptor-interacting proteins, transporters of ions or metabolites, ion channels, cell anchoring proteins, and cell type-specific surface antigens.

Many membrane proteins (MPs) contain amino acid sequence motifs that target these proteins to specific subcellular sites. Examples of these motifs include PDZ domains, KDEL, ROD, NOR, and GSL sequence motifs, von Willebrand factor A (vWFA) domains, and EGF-like domains. RGD, NGR, and GSL motif-containing peptides have been used as drug delivery agents in targeted cancer treatment of tumor vasculature (Arap, W. et al. (1998) Science 279:377-380). Furthermore, MPs may also contain amino acid sequence motifs, such as the carbohydrate recognition domain (CRD), that mediate interactions with extracellular or intracellular molecules.

G-Protein Coupled Receptors

G-protein coupled receptors (GPCR) are a superfamily of integral membrane proteins which transduce extracellular signals. GPCRs include receptors for biogenic amines, lipid mediators of inflammation, peptide hormones, and sensory signal mediators. The structure of these highly-conserved receptors consists of seven hydrophobic transmembrane regions, an extracellular N-terminus, and a cytoplasmic C-terminus. Three extracellular loops alternate with three intracellular loops to link the seven transmembrane regions. Cysteine disulfide bridges connect the second and third extracellular loops. The most conserved regions of GPCRs are the transmembrane regions and the first two cytoplasmic loops. A conserved, acidic-Arg-aromatic residue triplet present in the second cytoplasmic loop may interact with G proteins. A GPCR consensus pattern is characteristic of most proteins belonging to this superfamily (ExPASy PROSITE document PS00237; and Watson, S. and S. Arkinstall (1994) The G-protein Linked Receptor Facts Book, Academic Press, San Diego Calif., pp. 2-6). Mutations and changes in transcriptional activation of GPCR-encoding genes have been associated with neurological disorders such as schizophrenia, Parkinson's disease, Alzheimer's disease, drug addiction, and feeding disorders.

Scavenger Receptors

Macrophage scavenger receptors with broad ligand specificity may participate in the binding of low density lipoproteins (LDL) and foreign antigens. Scavenger receptors types I and II are trimeric membrane proteins with each subunit containing a small N-terminal intracellular domain, a transmembrane domain, a large extracellular domain, and a C-terminal cysteine-rich domain. The extracellular domain contains a short spacer region, an α-helical coiled-coil region, and a triple helical collagen-like region. These receptors have been shown to bind a spectrum of ligands, including chemically modified lipoproteins and albumin, polyribonucleotides, polysaccharides, phospholipids, and asbestos (Matsumoto, A. et al. (1990) Proc. Natl. Acad. Sci. USA 87:9133-9137; and Elomaa, 0. et al. (1995) Cell 80:603-609). The scavenger receptors are thought to play a key role in atherogenesis by mediating uptake of modified LDL in arterial walls, and in host defense by binding bacterial endotoxins, bacteria, and protozoa

Tetraspan Family Proteins

The transmembrane 4 superfamily (TM4SF) or tetraspan family is a multigene family encoding type III integral membrane proteins (Wright, AD. and M. G. Tomlinson (1994) Immunol.

Today 15:588-594). The TM4SF is comprised of membrane proteins which traverse the cell membrane four times. Members of the TM4SF include platelet and endothelial cell membrane proteins, melanoma-associated antigens, leukocyte surface glycoproteins, colonal carcinoma antigens, tumor-associated antigens, and surface proteins of the schistosome parasites (Jankowski, S. A. (1994) Oncogene 9:1205-1121). Members of the TM4SF share about 25-30% amino acid sequence identity with one another.

A number of TM4SF members have been implicated in signal transduction, control of cell adhesion, regulation of cell growth and proliferation, including development and oncogenesis, and cell motility, including tumor cell metastasis. Expression of TM4SF proteins is associated with a variety of tumors and the level of expression may be altered when cells are growing or activated.

Tumor Antigens

Tumor antigens are cell surface molecules that are differentially expressed in tumor cells relative to normal cells. Tumor antigens distinguish tumor cells immunologically from normal cells and provide diagnostic and therapeutic targets for human cancers (Takagi, S. et al. (1995) Int. J. Cancer 61:706-715; Liu, E. et al. (1992) Oncogene 7:1027-1032).

Leukocyte Antigens

Other types of cell surface antigens include those identified on leukocytic cells of the immune system. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “cluster of differentiation” or “CD” designation. Some of the genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylinositol (GPI). (Reviewed in Barclay, A. N. et al. (1995) The Leucocyte Antigen Facts Book, Academic Press, San Diego Calif., pp. 17-20.)

Ion Channels

Ion channels are found in the plasma membranes of virtually every cell in the body. For example, chloride channels mediate a variety of cellular functions including regulation of membrane potentials and absorption and secretion of ions across epithelial membranes. Chloride channels also regulate the pH of organelles such as the Golgi apparatus and endosomes (see, e.g., Greger, R. (1988)

Annu. Rev. Physiol. 50:111-122). Electrophysiological and pharmacological properties of chloride channels, including ion conductance, current-voltage relationships, and sensitivity to modulators, suggest that different chloride channels exist in muscles, neurons, fibroblasts, epithelial cells, and lymphocytes.

Many ion channels have sites for phosphorylation by one or more protein kinases including protein kinase A, protein kinase C, tyrosine kinase, and casein kinase II, all of which regulate ion channel activity in cells. Inappropriate phosphorylation of proteins in cells has been linked to changes in cell cycle progression and cell differentiation. Changes in the cell cycle have been linked to induction of apoptosis or cancer. Changes in cell differentiation have been linked to diseases and disorders of the reproductive system, immune system, skeletal muscle, and other organ systems.

Proton Pumps

Proton ATPases comprise a large class of membrane proteins that use the energy of ATP hydrolysis to generate an electrochemical proton gradient across a membrane. The resultant gradient may be used to transport other ions across the membrane (Na⁺, K⁺, or Cl⁻) or to maintain organelle pH. Proton ATPases are further subdivided into the mitochondrial F-ATPases, the plasma membrane ATPases, and the vacuolar ATPases. The vacuolar ATPases establish and maintain an acidic pH within various organelles involved in the processes of endocytosis and exocytosis (Mellman, I. et al. (1986) Annu. Rev. Biochem 55:663-700).

Proton-coupled, 12 membrane-spanning domain transporters such as PEPT 1 and PEPT 2 are responsible for gastrointestinal absorption and for renal reabsorption of peptides using an electrochemical H⁺ gradient as the driving force. Another type of peptide transporter, the TAP transporter, is a heterodimer consisting of TAP 1 and TAP 2 and is associated with antigen processing. Peptide antigens are transported across the membrane of the endoplasmic reticulum by TAP so they can be expressed on the cell surface in association with MHC molecules. Each TAP protein consists of multiple hydrophobic membrane spanning segments and a highly conserved ATP-binding cassette (Boll, M. et al. (1996) Proc. Natl. Acad. Sci. USA 93:284-289). Pathogenic microorganisms, such as herpes simplex virus, may encode inhibitors of TAP-mediated peptide transport in order to evade immune surveillance (Marusina, K and J. J. Manaco (1996) Curr. Opin.

Hematol. 3:19-26).

ABC Transporters

The ATP-binding cassette (ABC) transporters, also called the “traffic ATPases”, comprise a superfamily of membrane proteins that mediate transport and channel functions in prokaryotes and eukaryotes (Higgins, C. F. (1992) Annu. Rev. Cell Biol. 8:67-113). ABC proteins share a similar overall structure and significant sequence homology. All ABC proteins contain a conserved domain of approximately two hundred amino acid residues which includes one or more nucleotide binding domains. Mutations in ABC transporter genes are associated with various disorders, such as hyperbilirubinemia II/Dubin-Johnson syndrome, recessive Stargardt's disease, X-linked adrenoleukodystrophy, multidrug resistance, celiac disease, and cystic fibrosis.

Peripheral and Anchored Membrane Proteins

Some membrane proteins are not membrane-spanning but are attached to the plasma membrane via membrane anchors or interactions with integral membrane proteins. Membrane anchors are covalently joined to a protein post-translationally and include such moieties as prenyl, myristyl, and glycosylphosphatidyl inositol groups. Membrane localization of peripheral and anchored proteins is important for their function in processes such as receptor-mediated signal transduction. For example, prenylation of Ras is required for its localization to the plasma membrane and for its normal and oncogenic functions in signal transduction.

Vesicle Coat Proteins

Intercellular communication is essential for the development and survival of multicellular organisms. Cells communicate with one another through the secretion and uptake of protein signaling molecules. The uptake of proteins into the cell is achieved by the endocytic pathway, in which the interaction of extracellular signaling molecules with plasma membrane receptors results in the formation of plasma membrane-derived vesicles that enclose and transport the molecules into the cytosol. These transport vesicles fuse with and mature into endosomal and lysosomal (digestive) compartments. The secretion of proteins from the cell is achieved by exocytosis, in which molecules inside of the cell proceed through the secretory pathway. In this pathway, molecules transit from the ER to the Golgi apparatus and finally to the plasma membrane, where they are secreted from the cell.

Several steps in the transit of material along the secretory and endocytic pathways require the formation of transport vesicles. Specifically, vesicles form at the transitional endoplasmic reticulum (tER), the rim of Golgi cisternae, the face of the Trans-Golgi Network (TGN), the plasma membrane (M), and tubular extensions of the endosomes. Vesicle formation occurs when a region of membrane buds off from the donor organelle. The membrane-bound vesicle contains proteins to be transported and is surrounded by a proteinaceous coat, the components of which are recruited from the cytosol. Two different classes of coat protein have been identified. Clathrin coats form on vesicles derived from the TGN and PM, whereas coatomer (COP) coats form on vesicles derived from the ER and Golgi. COP coats can be further classified as COPI, involved in retrograde traffic through the Golgi and from the Golgi to the ER, and COPII, involved in anterograde traffic from the ER to the Golgi (Mellman supra).

In clathrin-based vesicle formation, adapter proteins bring vesicle cargo and coat proteins together at the surface of the budding membrane. Adapter protein-1 and -2 select cargo from the TGN and plasma membrane, respectively, based on molecular information encoded on the cytoplasmic tail of integral membrane cargo proteins. Adapter proteins also recruit clathrin to the bud site. Clathrin is a protein complex consisting of three large and three small polypeptide chains arranged in a three-legged structure called a triskelion. Multiple triskelions and other coat proteins appear to self-assemble on the membrane to form a coated pit. This assembly process may serve to deform the membrane into a budding vesicle. GTP-bound ADP-ribosylation factor (Arf) is also incorporated into the coated assembly. Another small G-protein, dynamin, forms a ring complex around the neck of the forming vesicle and may provide the mechanochemical force to seal the bud, thereby releasing the vesicle. The coated vesicle complex is then transported through the cytosol. During the transport process, Arf-bound GTP is hydrolyzed to GDP, and the coat dissociates from the transport vesicle (West, M. A. et al. (1997) J. Cell Biol. 138:1239-1254).

Vesicles which bud from the ER and the Golgi are covered with a protein coat similar to the clathrin coat of endocytic and TGN vesicles. The coat protein (COP) is assembled from cytosolic precursor molecules at specific budding regions on the organelle. The COP coat consists of two major components, a G-protein (Arf or Sar) and coat protomer (coatomer). Coatomer is an equimolar complex of seven proteins, termed alpha-, beta-, beta′-, gamma-, delta-, epsilon- and zeta-COP. The coatomer complex binds to dilysine motifs contained on the cytoplasmic tails of integral membrane proteins. These include the KKXX retrieval motif of membrane proteins of the ER and dibasic/diphenylamine motifs of members of the p24 family. The p24 family of type I membrane proteins represent the major membrane proteins of COPI vesicles (Harter, C. and F. T. Wieland (1998) Proc. Natl. Acad. Sci. USA 95:11649-11654).

Organelle Associated Molecules

Eukaryotic cells are organized into various cellular organelles which has the effect of separating specific molecules and their functions from one another and from the cytosol. Within the cell, various membrane structures surround and define these organelles while allowing them to interact with one another and the cell environment through both active and passive transport processes. Important cell organelles include the nucleus, the Golgi apparatus, the endoplasmic reticulum, mitochondria, peroxisomes, lysosomes, endosomes, and secretory vesicles.

Nucleus

The cell nucleus contains all of the genetic information of the cell in the form of DNA, and the components and machinery necessary for replication of DNA and for transcription of DNA into RNA. (See Alberts, B. et al. (1994) Molecular Biology of the Cell, Garland Publishing Inc., New York N.Y., pp. 335-399.) DNA is organized into compact structures in the nucleus by interactions with various DNA-binding proteins such as histones and non-histone chromosomal proteins. DNA-specific nucleases, DNAses, partially degrade these compacted structures prior to DNA replication or transcription. DNA replication takes place with the aid of DNA helicases which unwind the double-stranded DNA helix, and DNA polymerases that duplicate the separated DNA strands.

Transcriptional regulatory proteins are essential for the control of gene expression. Some of these proteins function as transcription factors that initiate, activate, repress, or terminate gene transcription. Transcription factors generally bind to the promoter, enhancer, and upstream regulatory regions of a gene in a sequence-specific manner, although some factors bind regulatory elements within or downstream of a gene's coding region. Transcription factors may bind to a specific region of DNA singly or as a complex with other accessory factors. (Reviewed in Lewin, B. (1990) Genes IV, Oxford University Press, New York N.Y., and Cell Press, Cambridge Mass., pp. 554570.) Many transcription factors incorporate DNA-binding structural motifs which comprise either α helices or β sheets that bind to the major groove of DNA. Four well-characterized structural motifs are helix-turn-helix, zinc finger, leucine zipper, and helix-loop-helix. Proteins containing these motifs may act alone as monomers, or they may form homo- or heterodimers that interact with DNA.

Many neoplastic disorders in humans can be attributed to inappropriate gene expression. Malignant cell growth may result from either excessive expression of tumor promoting genes or insufficient expression of tumor suppressor genes (Cleary, M. L. (1992) Cancer Surv. 15:89-104). Chromosomal translocations may also produce chimeric loci which fuse the coding sequence of one gene with the regulatory regions of a second unrelated gene. Such an arrangement likely results in inappropriate gene transcription, potentially contributing to malignancy.

In addition, the immune system responds to infection or trauma by activating a cascade of events that coordinate the progressive selection, amplification, and mobilization of cellular defense mechanisms. A complex and balanced program of gene activation and repression is involved in this process. However, hyperactivity of the immune system as a result of improper or insufficient regulation of gene expression may result in considerable tissue or organ damage. This damage is well documented in immunological responses associated with arthritis, allergens, heart attack, stroke, and infections (Isselbacher, K. J. et al. (1996) Harrison's Principles of Internal Medicine, 13/e, McGraw Hill, Inc. and Teton Data Systems Software).

Transcription of DNA into RNA also takes place in the nucleus catalyzed by RNA polymerases. Three types of RNA polymerase exist RNA polymerase I makes large ribosomal RNAs, while RNA polymerase m makes a variety of small, stable RNAs including 5S ribosomal RNA and the transfer RNAs (tRNA). RNA polymerase II transcribes genes that will be translated into proteins. The primary transcript of RNA polymerase II is called heterogenous nuclear RNA (hnRNA), and must be further processed by splicing to remove non-coding sequences called introns. RNA splicing is mediated by small nuclear ribonucleoprotein complexes, or snRNPs, producing mature messenger RNA (mRNA) which is then transported out of the nucleus for translation into proteins.

Nucleolus

The nucleolus is a highly organized subcompartment in the nucleus that contains high concentrations of RNA and proteins and functions mainly in ribosomal RNA synthesis and assembly (Alberts, et al. supra, pp. 379-382). Ribosomal RNA (rRNA) is a structural RNA that is complexed with proteins to form ribonucleoprotein structures called ribosomes. Ribosomes provide the platform on which protein synthesis takes place.

Ribosomes are assembled in the nucleolus initially from a large, 45S rRNA combined with a variety of proteins imported from the cytoplasm, as well as smaller, 5S rRNAs. Later processing of the immature ribosome results in formation of smaller ribosomal subunits which are transported from the nucleolus to the cytoplasm where they are assembled into functional ribosomes.

Endoplasmic Reticulum

In eukaryotes, proteins are synthesized within the endoplasmic reticulum (ER), delivered from the ER to the Golgi apparatus for post-translational processing and sorting, and transported from the Golgi to specific intracellular and extracellular destinations. Synthesis of integral membrane proteins, secreted proteins, and proteins destined for the lumen of a particular organelle occurs on the rough endoplasmic reticulum (ER). The rough ER is so named because of the rough appearance in electron micrographs imparted by the attached ribosomes on which protein synthesis proceeds. Synthesis of proteins destined for the ER actually begins in the cytosol with the synthesis of a specific signal peptide which directs the growing polypeptide and its attached ribosome to the ER membrane where the signal peptide is removed and protein synthesis is completed. Soluble proteins destined for the ER lumen, for secretion, or for transport to the lumen of other organelles pass completely into the ER lumen. Transmembrane proteins destined for the ER or for other cell membranes are translocated across the ER membrane but remain anchored in the lipid bilayer of the membrane by one or more membrane-spanning α-helical regions.

Translocated polypeptide chains destined for other organelles or for secretion also fold and assemble in the ER lumen with the aid of certain “resident” ER proteins. Protein folding in the ER is aided by two principal types of protein isomerases, protein disulfide isomerase (PDI), and peptidyl-prolyl isomerase (PPI). PDI catalyzes the oxidation of free sulfhdryl groups in cysteine residues to form intramolecular disulfide bonds in proteins. PPI, an enzyme that catalyzes the isomerization of certain proline imide bonds in oligopeptides and proteins, is considered to govern one of the rate limiting steps in the folding of many proteins to their final functional conformation. The cyclophilins represent a major class of PPI that was originally identified as the major receptor for the immunosuppressive drug cyclosporin A (Handschumacher, R. E. et al. (1984) Science 226:544-547). Molecular “chaperones” such as BiP (binding protein) in the ER recognize incorrectly folded proteins as well as proteins not yet folded into their final form and bind to them, both to prevent improper aggregation between them, and to promote proper folding.

The “N-linked” glycosylation of most soluble secreted and membrane-bound proteins by oligosacchrides linked to asparagine residues in proteins is also performed in the ER. This reaction is catalyzed by a membrane-bound enzyme, oligosaccharyl transferase.

Golgi Apparatus

The Golgi apparatus is a complex structure that lies adjacent to the ER in eukaryotic cells and serves primarily as a sorting and dispatching station for products of the ER (Alberts, et al. supra, pp. 600-610). Additional posttranslational processing, principally additional glycosylation, also occurs in the Golgi. Indeed, the Golgi is a major site of carbohydrate synthesis, including most of the glycosaminoglycans of the extracellular matrix. N-linked oligosaccharides, added to proteins in the ER, are also further modified in the Golgi by the addition of more sugar residues to form complex N-linked oligosaccharides. “O-linked” glycosylation of proteins also occurs in the Golgi by the addition of N-acetylgalactosamine to the hydroxyl group of a serine or threonine residue followed by the sequential addition of other sugar residues to the first. This process is catalyzed by a series of glycosyltransferases each specific for a particular donor sugar nucleotide and acceptor molecule (Lodish, H. et al. (1995) Molecular Cell Biology, W. H. Freeman and Co., New York N.Y., pp.700-708). In many cases, both N- and O-linked oligosaccharides appear to be required for the secretion of proteins or the movement of plasma membrane glycoproteins to the cell surface.

The terminal compartment of the Golgi is the Trans-Golgi Network (TGN), where both membrane and lumenal proteins are sorted for their final destination. Transport (or secretory) vesicles destined for intracellular compartments, such as lysosomes, bud off of the TGN. Other transport vesicles bud off containing proteins destined for the plasma membrane, such as receptors, adhesion molecules, and ion channels, and secretory proteins, such as hormones, neurotransmitters, and digestive enzymes.

Vacuoles

The vacuole system is a collection of membrane bound compartments in eukaryotic cells that functions in the processes of endocytosis and exocytosis. They include phagosomes, lysosomes, endosomes, and secretory vesicles. Endocytosis is the process in cells of internalizing nutrients, solutes or small particles (pinocytosis) or large particles such as internalized receptors, viruses, bacteria, or bacterial toxins (phagocytosis). Exocytosis is the process of transporting molecules to the cell surface. It facilitates placement or localization of membrane-bound receptors or other membrane proteins and secretion of hormones, neurotransmitters, digestive enzymes, wastes, etc.

A common property of all of these vacuoles is an acidic pH environment ranging from approximately pH 4.5-5.0. This acidity is maintained by the presence of a proton ATPase that uses the energy of ATP hydrolysis to generate an electrochemical proton gradient across a membrane (Mellman, I. et al. (1986) Annu. Rev. Biochem. 55:663-700). Eukaryotic vacuolar proton ATPase (vp-ATPase) is a multimeric enzyme composed of 3-10 different subunits. One of these subunits is a highly hydrophobic polypeptide of approximately 16 kDa that is similar to the proteolipid component of vp-ATPases from eubacteria, fungi, and plant vacuoles (Mandel, M. et al. (1988) Proc. Natl. Acad. Sci. USA 85:5521-5524). The 16 kDa proteolipid component is the major subunit of the membrane portion of vp-ATPase and functions in the transport of protons across the membrane.

Lysosomes

Lysosomes are membranous vesicles containing various hydrolytic enzymes used for the controlled intracellular digestion of macromolecules. Lysosomes contain some 40 types of enzymes including proteases, nucleases, glycosidases, lipases, phospholipases, phosphatases, and sulfatases, all of which are acid hydrolases that function at a pH of about 5. Lysosomes are surrounded by a unique membrane containing transport proteins that allow the final products of macromolecule degradation, such as sugars, amino acids, and nucleotides, to be transported to the cytosol where they may be either excreted or reutilized by the cell. A vp-ATPase, such as that described above, maintains the acidic environment necessary for hydrolytic activity (Alberts, supra, pp.610-611).

Endosomes

Endosomes are another type of acidic vacuole that is used to transport substances from the cell surface to the interior of the cell in the process of endocytosis. Like lysosomes, endosomes have an acidic environment provided by a vp-ATPase (Alberts et al. supra pp. 610-618). Two types of endosomes are apparent based on tracer uptake studies that distinguish their time of formation in the cell and their cellular location. Early endosomes are found near the plasma membrane and appear to function primarily in the recycling of internalized receptors back to the cell surface. Late endosomes appear later in the endocytic process close to the Golgi apparatus and the nucleus, and appear to be associated with delivery of endocytosed material to lysosomes or to the TGN where they may be recycled. Specific proteins are associated with particular transport vesicles and their target compartments that may provide selectivity in targeting vesicles to their proper compartments. A cytosolic prenylated GTP-binding protein, Rab, is one such protein. Rabs 4, 5, and 11 are associated with the early endosome, whereas Rabs 7 and 9 associate with the late endosome.

Mitochondria

Mitochondria are oval-shaped organelles comprising an outer membrane, a tightly folded inner membrane, an intermembrane space between the outer and inner membranes, and a matrix inside the inner membrane. The outer membrane contains many porin molecules that allow ions and charged molecules to enter the intermembrane space, while the inner membrane contains a variety of transport proteins that transfer only selected molecules. Mitochondria are the primary sites of energy production in cells.

Energy is produced by the oxidation of glucose and fatty acids. Glucose is initially converted to pyruvate in the cytoplasm. Fatty acids and pyruvate are transported to the mitochondria for complete oxidation to CO₂ coupled by enzymes to the transport of electrons from NADH and FADH₂ to oxygen and to the synthesis of ATP (oxidative phosphorylation) from ADP and Pᵢ, Pyruvate is transported into the mitochondria and converted to acetyl-CoA for oxidation via the citric acid cycle, involving pyruvate dehydrogenase components, dihydrolipoyl transacetylase, and dihydrolipoyl dehydrogenase. Enzymes involved in the citric acid cycle include: citrate synthetase, aconitases, isocitrate dehydrogenase, alpha-ketoglutarate dehydrogenase complex including transsuccinylases, succinyl CoA synthetase, succinate dehydrogenase, fumarases, and malate dehydrogenase. Acetyl CoA is oxidized to CO₂ with concomitant formation of NADH, FADH₂, and GTP. In oxidative phosphorylation, the transfer of electrons from NADH and FADH₂ to oxygen by dehydrogenases is coupled to the synthesis of ATP from ADP and Pᵢ by the F₀F₁ ATPase complex in the mitochondrial inner membrane. Enzyme complexes responsible for electron transport and ATP synthesis include the F₀F₁ ATPase complex, ubiquinone(CoQ)-cytochrome c reductase, ubiquinone reductase, cytochrome b, cytochrome c₁, FeS protein, and cytochrome c oxidase.

Peroxisomes

Peroxisomes, like mitochondria, are a major site of oxygen utilization. They contain one or more enzymes, such as catalase and urate oxidase, that use molecular oxygen to remove hydrogen atoms from specific organic substrates in an oxidative reaction that produces hydrogen peroxide (Alberts, supra, pp. 574577). Catalase oxidizes a variety of substrates including phenols, formic acid, formaldehyde, and alcohol and is important in peroxisomes of liver and kidney cells for detoxifying various toxic molecules that enter the bloodstream. Another major function of oxidative reactions in peroxisomes is the breakdown of fatty acids in a process called β oxidation. β oxidation results in shortening of the alkyl chain of fatty acids by blocks of two carbon atoms that are converted to acetyl CoA and exported to the cytosol for reuse in biosynthetic reactions.

Also like mitochondria, peroxisomes import their proteins from the cytosol using a specific signal sequence located near the C-terminus of the protein. The importance of this import process is evident in the inherited human disease Zellweger syndrome, in which a defect in importing proteins into perixosomes leads to a perixosomal deficiency resulting in severe abnormalities in the brain liver, and kidneys, and death soon after birth. One form of this disease has been shown to be due to a mutation in the gene encoding a perixosomal integral membrane protein called peroxisome assembly factor-1.

The discovery of new human molecules satisfies a need in the art by providing new compositions which are useful in the diagnosis, study, prevention, and treatment of diseases associated with, as well as effects of exogenous compounds on, the expression of human molecules.

### SUMMARY OF THE INVENTION

The present invention relates to nucleic acid sequences comprising human diagnostic and therapeutic polynucleotides (dithp) as presented in the Sequence Listing. The dithp uniquely identify genes encoding human structural, functional, and regulatory molecules.

The invention provides an isolated polynucleotide comprising a polynucleotide sequence selected from the group consisting of a) a polynucleotide sequence selected from the group consisting of SEQ ID NO:1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). In one alternative, the polynucleotide comprises a polynucleotide sequence selected from the group consisting of SEQ ID NO:1-211. In another alternative, the polynucleotide comprises at least 60 contiguous nucleotides of a polynucleotide sequence selected from the group consisting of a) a polynucleotide sequence selected from the group consisting of SEQ ID NO: 1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). The invention further provides a composition for the detection of expression of human diagnostic and therapeutic polynucleotides comprising at least one isolated polynucleotide comprising a polynucleotide sequence selected from the group consisting of a) a polynucleotide sequence selected from the group consisting of SEQ ID NO: 1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d); and a detectable label.

The invention also provides a method for detecting a target polynucleotide in a sample, said target polynucleotide comprising a polynucleotide sequence selected from the group consisting of a) a polynucleotide sequence selected from the group consisting of SEQ ID NO:1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). The method comprises a) amplifying said target polynucleotide or a fragment thereof using polymerase chain reaction amplification, and b) detecting the presence or absence of said amplified target polynucleotide or fragment thereof, and, optionally, if present, the amount thereof.

The invention also provides a method for detecting a target polynucleotide in a sample, said target polynucleotide comprising a polynucleotide sequence selected from the group consisting of a) a polynucleotide sequence selected from the group consisting of SEQ ID NO:1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide, and b) detecting the presence or absence of said hybridization complex, and, optionally, if present, the amount thereof. In one alternative, the probe comprises at least 30 contiguous nucleotides. In another alternative, the probe comprises at least 60 contiguous nucleotides.

The invention further provides a recombinant polynucleotide comprising a promoter sequence operably linked to an isolated polynucleotide comprising a polynucleotide sequence selected from the group consisting of a) a polynucleotide sequence selected from the group consisting of SEQ ID NO:1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). In one alternative, the invention provides a cell transformed with the recombinant polynucleotide. In another alternative, the invention provides a transgenic organism comprising the recombinant polynucleotide. In a further alternative, the invention provides a method for producing a human diagnostic and therapeutic polypeptide, the method comprising a) culturing a cell under conditions suitable for expression of the human diagnostic and therapeutic polypeptide, wherein said cell is transformed with the recombinant polynucleotide, and b) recovering the human diagnostic and therapeutic polypeptide so expressed.

The invention also provides a purified human diagnostic and therapeutic polypeptide (DITHP) encoded by at least one polynucleotide comprising a polynucleotide sequence selected from the group consisting of SEQ ID NO:1-211. Additionally, the invention provides an isolated antibody which specifically binds to the human diagnostic and therapeutic polypeptide. The invention further provides a method of identifying a test compound which specifically binds to the human diagnostic and therapeutic polypeptide, the method comprising the steps of a) providing a test compound; b) combining the human diagnostic and therapeutic polypeptide with the test compound for a sufficient time and under suitable conditions for binding; and c) detecting binding of the human diagnostic and therapeutic polypeptide to the test compound, thereby identifying the test compound which specifically binds the human diagnostic and therapeutic polypeptide.

The invention further provides a microarray wherein at least one element of the microarray is an isolated polynucleotide comprising at least 60 contiguous nucleotides of a polynucleotide comprising a polynucleotide sequence selected from the group consisting of a) a polynucleotide sequence selected from the group consisting of SEQ ID NO:1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). The invention also provides a method for generating a transcript image of a sample which contains polynucleotides. The method comprises a) labeling the polynucleotides of the sample, b) contacting the elements of the microarray with the labeled polynucleotides of the sample under conditions suitable for the formation of a hybridization complex, and c) quantifying the expression of the polynucleotides in the sample.

Additionally, the invention provides a method for screening a compound for effectiveness in altering expression of a target polynucleotide, wherein said target polynucleotide comprises a polynucleotide sequence selected from the group consisting of a) a polynucleotide sequence selected from the group consisting of SEQ ID NO:1-211; b) a naturally occurring polynucleotide sequence having at least 90% sequence identity to a polynucleotide sequence selected from the group consisting of SEQ ID NO:1-211; c) a polynucleotide sequence complementary to a); d) a polynucleotide sequence complementary to b); and e) an RNA equivalent of a) through d). The method comprises a) exposing a sample comprising the target polynucleotide to a compound, and b) detecting altered expression of the target polynucleotide, and c) comparing the expression of the target polynucleotide in the presence of varying amounts of the compound and in the absence of the compound

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide comprising a polynucleotide sequence selected from the group consisting of i) a polynucleotide sequence selected from the group consisting of SEQ ID NO:1-211; ii) a naturally occurring polynucleotide sequence having at least 90% sequence identity to a polynucleotide sequence selected from the group consisting of SEQ ID NO:1-211; iii) a polynucleotide sequence complementary to i), iv) a polynucleotide sequence complementary to ii), and v) an RNA equivalent of i)-iv). Hybridization occurs under conditions whereby a specific hybridization complex is formed between said probe and a target polynucleotide in the biological sample, said target polynucleotide comprising a polynucleotide sequence selected from the group consisting of i) a polynucleotide sequence selected from the group consisting of SEQ ID NO:1-211; ii) a naturally occurring polynucleotide sequence having at least 90% sequence identity to a polynucleotide sequence selected from the group consisting of SEQ ID NO:1-211; iii) a polynucleotide sequence complementary to i), iv) a polynucleotide sequence complementary to ii), and v) an RNA equivalent of i)-iv), and alternatively, the target polynucleotide comprises a fragment of a polynucleotide sequence selected from the group consisting of i-v above; c) quantifying the amount of hybridization complex; and d) comparing the amount of hybridization complex in the treated biological sample with the amount. of hybridization complex in an untreated biological sample, wherein a difference in the amount of hybridization complex in the treated biological sample is indicative of toxicity of the test compound. The invention further provides an isolated polypeptide comprising an amino acid sequence selected from the group consisting of a) an amino acid sequence selected from the group consisting of SEQ ID NO:212-422, b) a naturally occurring amino acid sequence having at least 90% sequence identity to an amino acid sequence selected from the group consisting of SEQ ID NO:212-422, c) a biologically active fragment of an amino acid sequence selected from the group consisting of SEQ ID NO:212-422, and d) an inmmunogenic fragment of an amino acid sequence selected from the group consisting of SEQ ID NO:212-422. In one alternative, the invention provides an isolated polypeptide comprising the amino acid sequence of SEQ ID NO:212-422.

### DESCRIPTION OF THE TABLES

Table 1 shows the sequence identification numbers (SEQ ID NO:s) and template identification numbers (template IDs) corresponding to the polynucleotides of the present invention, along with their GenBank hits (GI Numbers), probability scores, and functional annotations corresponding to the GenBank hits.

Table 2 shows the sequence identification numbers (SEQ ID NO:s) and template identification numbers (template IDs) corresponding to the polynucleotides of the present invention, along with polynucleotide segments of each template sequence as defined by the indicated “start” and “stop” nucleotide positions. The reading frames of the polynucleotide segments and the Pfam bits, Pfam descriptions, and E-values corresponding to the polypeptide domains encoded by the polynucleotide segments are indicated.

Table 3 shows the sequence identification numbers (SEQ ID NO:s) and template identification numbers (template IDs) corresponding to the polynucleotides of the present invention, along with polynucleotide segments of each template sequence as defined by the indicated “start and “stop” nucleotide positions. The reading frames of the polynucleotide segments are shown, and the polypeptides encoded by the polynucleotide segments constitute either signal peptide (SP) or transmembrane (TM) domains, as indicated. The membrane topology of the encoded polypeptide sequence is indicated, the N-terminus (N) listed as being oriented to either the cytosolic (in) or non-cytosolic (out) side of the cell membrane or organelle.

Table 4 shows the sequence identification numbers (SEQ ID NO:s) corresponding to the polynucleotides of the present invention, along with component sequence identification numbers (component IDs) corresponding to each template. The component sequences, which were used to assemble the template sequences, are defined by the indicated “start” and “stop” nucleotide positions along each template.

Table 5 shows the tissue distribution profiles for the templates of the invention Table 6 shows the sequence identification numbers (SEQ ID NO:s) corresponding to the polypeptides of the present invention, along with the reading frames used to obtain the polypeptide segments, the lengths of the polypeptide segments, the “start” and “stop” nucleotide positions of the polynucleotide sequences used to define the encoded polypeptide segments, the GenBank hits (GI Numbers), probability scores, and functional annotations corresponding to the GenBank hits.

Table 7 summarizes the bioinformatics tools which are useful for analysis of the polynucleotides of the present invention. The first column of Table 7 lists analytical tools, programs, and algorithms, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences).

### DETAILED DESCRIPTION OF THE INVENTION

Before the nucleic acid sequences and methods are presented, it is to be understood that this invention is not limited to the particular machines, methods, and materials described. Although particular embodiments are described, machines, methods, and materials similar or equivalent to these embodiments may be used to practice the invention. The preferred machines, methods, and materials set forth are not intended to limit the scope of the invention which is limited only by the appended claims.

The singular forms “a”, “an”, and “the” include plural reference unless the context clearly dictates otherwise. AR technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

As used herein, the lower case “ditmp” refers to a nucleic acid sequence, while the upper case “DrnHp” refers to an amino acid sequence encoded by dithp. A “full-length” dithp refers to a nucleic acid sequence containing the entire coding region of a gene endogenously expressed in human tissue.

“Adjuvants” are materials such as Freund's adjuvant, mineral gels (aluminum hydroxide), and surface active substances (lysolecithin, pluronic polyols, polyanions, peptides, oil emulsions, keyhole limpet hemocyanin, and dinitrophenol) which may be administered to increase a host's immunological response.

“Allele” refers to an alternative form of a nucleic acid sequence. Alleles result from a “mutation,” a change or an alternative reading of the genetic code. Any given gene may have none, one, or many allelic forms. Mutations which give rise to alleles include deletions, additions, or substitutions of nucleotides. Each of these changes may occur alone, or in combination with the others, one or more times in a given nucleic acid sequence. The present invention encompasses allelic dithp.

“Amino acid sequence” refers to a peptide, a polypeptide, or a protein of either natural or synthetic origin. The amino acid sequence is not limited to the complete, endogenous amino acid sequence and may be a fragment, epitope, variant, or derivative of a protein expressed by a nucleic acid sequence.

“Amplification” refers to the production of additional copies of a sequence and is carried out using polymerase chain reaction (PCR) technologies well known in the art.

“Antibody” refers to intact molecules as well as to fragments thereof, such as Fab, F(ab′)₂, and Fv fragments, which are capable of binding the epitopic determine. Antibodies that bind DITHP polypeptides can be prepared using intact polypeptides or using fragments containing small peptides of interest as the immunizing antigen. The polypeptide or peptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

“Antisense sequence” refers to a sequence capable of specifically hybridizing to a target sequence. The antisense sequence may include DNA, RNA, or any nucleic acid mimic or analog such as peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine.

“Antisense sequence” refers to a sequence capable of specifically hybridizing to a target sequence. The antisense sequence can be DNA, RNA, or any nucleic acid mimic or analog.

“Antisense technology” refers to any technology which relies on the specific hybridization of an antisense sequence to a target sequence.

A “bin” is a portion of computer memory space used by a computer program for storage of data, and bounded in such a manner that data stored in a bin may be retrieved by the program.

“Biologically active” refers to an amino acid sequence having a structural, regulatory, or biochemical function of a naturally occurring amino acid sequence.

“Clone joining” is a process for combining gene bins based upon the bins' containing sequence information from the same clone. The sequences may assemble into a primary gene transcript as well as one or more splice variants.

“Complementary” describes the relationship between two single-stranded nucleic acid sequences that anneal by base-pairing (5′-A-G-T-3′ pairs with its complement 3′-T-C-A-5).

A “component sequence” is a nucleic acid sequence selected by a computer program such as PHRED and used to assemble a consensus or template sequence from one or more component sequences.

A “consensus sequence” or “template sequence” is a nucleic acid sequence which has been assembled from overlapping sequences, using a computer program for fragment assembly such as the GEL VIEW fragment assembly system (Genetics Computer Group (GCG), Madison Wis.) or using a relational database management system (RDMS).

“Conservative amino acid substitutions” are those substitutions that, when made, least interfere with the properties of the original protein, i.e., the structure and especially the function of the protein is conserved and not significantly changed by such substitutions. The table below shows amino acids which may be substituted for an original amino acid in a protein and which are regarded as conservative substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys,Thr |
| Thr | Ser, Val |
| Trp | Phe, Tyr |
| Tyr | His, Phe, Trp |
| Val | Ile, Leu, Thr |

Conservative substitutions generally maintain (a) the structure of the polypeptide backbone in the area of the substitution, for example, as a beta sheet or alpha helical conformation, (b) the charge or hydrophobicity of the molecule at the target site, or (c) the bulk of the side chain

“Deletion” refers to a change in either a nucleic or amino acid sequence in which at least one nucleotide or amino acid residue, respectively, is absent

“Derivative” refers to the chemical modification of a nucleic acid sequence, such as by replacement of hydrogen by an alkyl, acyl, amino, hydroxyl, or other group.

The terms “element” and “array element” refer to a polynucleotide, polypeptide, or other chemical compound having a unique and defined position on a microarray.

“E-value” refers to the statistical probability that a match between two sequences occurred by chance.

A “fragment” is a unique portion of dithp or DITHP which is identical in sequence to but shorter in length than the parent sequence. A fragment may comprise up to the entire length of the defined sequence, minus one nucleotide/amino acid residue. For example, a fragment may comprise from 10 to 1000 contiguous amino acid residues or nucleotides. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous amino acid residues or nucleotides in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing and the figures, may be encompassed by the present embodiments.

A fragment of dithp comprises a region of unique polynucleotide sequence that specifically identifies dithp, for example, as distinct from any other sequence in the same genome. A fragment of dithp is useful, for example, in hybridization and amplification technologies and in analogous methods that distinguish dithp from related polynucleotide sequences. The precise length of a fragment of dithp and the region of dithp to which the fragment corresponds are routinely determinable by one of ordinary skill in the art based on the intended purpose for the fragment

A fragment of DITHP is encoded by a fragment of dithp. A fragment of DITHP comprises a region of unique amino acid sequence that specifically identifies DITHP. For example, a fragment of DITHP is useful as an immunogenic peptide for the development of antibodies that specifically recognize DITHP. The precise length of a fragment of DITHP and the region of DITHP to which the fragment corresponds are routinely determinable by one of ordinary skill in the art based on the intended purpose for the fragment.

A “full length” nucleotide sequence is one containing at least a start site for translation to a protein sequence, followed by an open reading frame and a stop site, and encoding a “full length” polypeptide.

“Hit” refers to a sequence whose annotation will be used to describe a given template. Criteria for selecting the top hit are as follows: if the template has one or more exact nucleic acid matches, the top hit is the exact match with highest percent identity. If the template has no exact matches but has significant protein hits, the top hit is the protein hit with the lowest E-value. If the template has no significant protein hits, but does have significant non-exact nucleotide hits, the top hit is the nucleotide hit with the lowest E-value.

“Homology” refers to sequence similarity either between a reference nucleic acid sequence and at least a fragment of a dithp or between a reference amino acid sequence and a fragment of a DITHP.

“Hybridization” refers to the process by which a strand of nucleotides anneals with a complementary strand through base pairing. Specific hybridization is an indication that two nucleic acid sequences share a high degree of identity. Specific hybridization complexes form under defined annealing conditions, and remain hybridized after the “washing” step. The defined hybridization conditions include the annealing conditions and the washing step(s), the latter of which is particularly important in determining the stringency of the hybridization process, with more stringent conditions allowing less non-specific binding, i.e., binding between pairs of nucleic acid probes that are not perfectly matched. Permissive conditions for annealing of nucleic acid sequences are routinely determinable and may be consistent among hybridization experiments, whereas wash conditions may be varied among experiments to achieve the desired stringency.

Generally, stringency of hybridization is expressed with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization is well known and can be found in Sambrook et al., 1989, Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., or 55° C. may be used. SSC concentration may be varied from about 0.2 to 2×SSC, with SDS being present at about 0.1%. Typically, blocking reagents are used to block non-specific hybridization. Such blocking reagents include, for instance, denatured salmon sperm DNA at about 100-200 μg/ml. Useful variations on these conditions will be readily apparent to those skilled in the art. Hybridization, particularly under high stringency conditions, may be suggestive of evolutionary similarity between the nucleotides. Such similarity is strongly indicative of a similar role for the nucleotides and their resultant proteins.

Other parameters, such as temperature, salt concentration; and detergent concentration may be varied to achieve the desired stringency. Denaturants, such as formamide at a concentration of about 35-50% v/v, may also be used under particular circumstanes, such as RNA:DNA hybridizations. Appropriate hybridization conditions are routinely determinable by one of ordinary skill in the art.

“Immunogenic” describes the potential for a natural, recombinant, or synthetic peptide, epitope, polypeptide, or protein to induce antibody production in appropriate animals, cells, or cell lines.

“Insertion” or “addition” refers to a change in either a nucleic or amino acid sequence in which at least one nucleotide or residue, respectively, is added to the sequence.

“Labeling” refers to the covalent or noncovalent joining of a polynucleotide, polypeptide, or antibody with a reporter molecule capable of producing a detectable or measurable signal.

Microarray” is any arrangement of nucleic acids, amino acids, antibodies, etc., on a substrate. The substrate may be a solid support such as beads, glass, paper, nitrocellulose, nylon, or an appropriate membrane.

“Linkers” are short stretches of nucleotide sequence which may be added to a vector or a dithp to create restriction endonuclease sites to facilitate cloning. “Polylinkers” are engineered to incorporate multiple restriction enzyme sites and to provide for the use of enzymes which leave 5′ or 3′ overhangs (e.g., BamHI, EcoRI, and HindIII) and those which provide blunt ends (e.g., EcoRV, SnaBI, and StuI).

“Naturally occurring” refers to an endogenous polynucleotide or polypeptide that may be isolated from viruses or prokaryotic or eukaryotic cells.

“Nucleic acid sequence” refers to the specific order of nucleotides joined by phosphodiester bonds in a linear, polymeric arrangement. Depending on the number of nucleotides, the nucleic acid sequence can be considered an oligomer, oligonucleotide, or polynucleotide. The nucleic acid can be DNA, RNA, or any nucleic acid analog, such as PNA, may be of genomic or synthetic origin, may be either double-stranded or single-stranded, and can represent either the sense or antisense (complementary) strand.

“Oligomer” refers to a nucleic acid sequence of at least about 6 nucleotides and as many as about 60 nucleotides, preferably about 15 to 40 nucleotides, and most preferably between about 20 and 30 nucleotides, that may be used in hybridization or amplification technologies. Oligomers may be used as, e.g., primers for PCR, and are usually chemically synthesized.

“Operably “inked” refers to the situation in which a first nucleic acid sequence is placed in a functional relationship with the second nucleic acid sequence. For instance, a promoter is operably lined to a coding sequence if the promoter affects the transcription or expression of the coding sequence. Generally, operably linked DNA sequences may be in close proximity or contiguous and, where necessary to join two protein coding regions, in the same reading frame.

“Peptide nucleic acid” (PNA) refers to a DNA mimic in which nucleotide bases are attached to a pseudopeptide backbone to increase stability. PNAs, also designated antigene agents, can prevent gene expression by targeting complementary messenger RNA

The phrases “percent identify” and “% identity”, as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences ma” be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and Sharp, P. M. (1989) CABIOS 5:151-153 and in Higgins, D. G. et al. (1992) CABIOS 8:189-191. For pairwise alignments of polynucleotide sequences, the default parameters are set as follows: Ktuple=2, gap penalty-5, window=4, and “diagonals saved”=4. The “weighted” residue weight table is selected as the default Percent identity is reported by CLUSTAL V as the “percent similarity” between aligned polynucleotide sequence pairs.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http:/www.ncbi.nlm.nih.gov/BLAST/. The BLAST software suite includes various sequence analysis programs including “blasta,” that is used to determine alignment between a known polynucleotide sequence and other sequences on a variety of databases. Also available is a tool called “BLAST 2 Sequences” that is used for direct pairwise comparison of two nucleotide sequences. “BLAST 2 Sequences” can be accessed and used interactively at http:/www.ncbi.nlm.nih.gov/gorf/bl2/. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May 7, 1999) set at default parameters. Such default parameters may be, for example:

Matrx: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in figures or Sequence Listings, may be used to describe a length over which percentage identity may be measured

Nucleic acid sequences that do not show a high degree of identity may nevertheless encode similar amino acid sequences due to the degeneracy of the genetic code. It is understood that changes in nucleic acid sequence can be made using this degeneracy to produce multiple nucleic acid sequences that all encode substantially the same protein.

The phrases “percent identity” and “% identity”, as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the hydrophobicity and acidity of the substituted residue, thus preserving the structure (and therefore function) of the folded polypeptide.

Percent identity between polypeptide sequences may be determined using the default parameters of the CLUSTAL V algorithm as incorporated into the MEGALIGN version 3.12e sequence alignment program (descried and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used For example, for a pairwise comparison of two polypeptide sequences, one may use the “BLAST 2 Sequences” tool Version 2.0.9 (May 7, 1999) with blastp set at default parameters. Such default parameters may be, for example.

Matrix: BLOSUM62

Open Gap: 11 and Extension Gap: 1 penalty

Gap x drop-off 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in figures or Sequence Listings, may be used to describe a length over which percentage identity may be measured.

“Post-translational modification” of a DITHP may involve lipidation, glycosylation, phosphorylation, acetylation, racemization, proteolytic cleavage, and other modifications known in the art. These processes may occur synthetically or biochemically. Biochemical modifications win vary by cell type depending on the enzymatic milieu and the DITHP.

“Probe” refers to dithp or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the figures and Sequence Listing, may be used.

Methods for preparing and using probes and primers are described in the references, for example Sambrook et al., 1989, Molecular Cloning: A Laboratory Manual, 2^{nd} ed., vol. 1-3, Cold Spring Harbor Press, Plainview N.Y.; Ausubel et al., 1987, Current Protocols in Molecular Biology, Greene Publ. Assoc. & Wiley-Intersciences, New York N.Y.; Innis et al., 1990, PCR Protocols. A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas T.x.) is capable of choosing specific primers from megabase sequences and is thus useful for designing primers on a genome-wide scope. The Primer3 primer selection program (available to the public from the Whitehead institute/MIT Center for Genome Research, Cambridge Mass.) allows the user to input a “mispriming library,” in which sequences to avoid as primer binding sites are user-specified Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

“Purified” refers to molecules, either polynucleotides or polypeptides that are isolated or separated from their natural environment and are at least 60% free, preferably at least 75% free, and most preferably at least 90% free from other compounds with which they are naturally associated.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

“Regulatory element” refers to a nucleic acid sequence from nontranslated regions of a gene, and includes enhancers, promoters, introns, and 3′ untranslated regions, which interact with host proteins to carry out or regulate transcription or translation

“Reporter” molecules are chemical or biochemical moieties used for labeling a nucleic acid, an amino acid, or an antibody. They include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

“Sample” is used in its broadest sense. Samples may contain nucleic or amino acids, antibodies, or other materials, and may be derived from any source (e.g., bodily fluids including, but not limited to, saliva, blood, and urine; chromosome(s), organelles, or membranes isolated from a cell; genomic DNA, RNA, or cDNA in solution or bound to a substrate; and cleared cells or tissues or blots or imprints from such cells or tissues).

“Specific binding” or “specifically binding” refers to the interaction between a protein or peptide and its agonist, antibody, antagonist, or other binding partner. The interaction is dependent upon the presence of a particular structure of the protein, e.g., the antigenic determinant or epitope, recognized by the binding molecule. For example, if an antibody is specific for epitope “A,” the presence of a polypeptide containing epitope A, or the presence of free unlabeled A, in a reaction containing free labeled A and the antibody will reduce the amount of labeled A that binds to the antibody.

“Substitution” refers to the replacement of at least one nucleotide or amino acid by a different nucleotide or amino acid.

“Substrate” refers to any suitable rigid or semi-rigid support including, e.g., membranes, filters, chips, slides, wafers, fibers, magnetic or nonmagnetic beads, gels, tubing, plates, polymers, microparticles or capillaries. The substrate can have a variety of surface forms, such as wells, trenches, pins, channels and pores, to which polynucleotides or polypeptides are bound.

A “transcript image” refers to the collective pattern of gene expression by a particular tissue or cell type under given conditions at a given time.

“Transformation” refers to a process by which exogenous DNA enters a recipient cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed.

“Transformants” include stably transformed cells in which the inserted DNA is capable of replication either as an autonomously replicating plasmid or as part of the host chromosome, as well as cells which transiently express inserted DNA or RNA.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, and plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 25% sequence identity to the particular nucleic acid sequence over a certain length of one of the is nucleic acid sequences using blastn with the “BLAST 2 Sequences” tool Version 2.0.9 (May 7, 1999) set at default parameters. Such a pair of nucleic acids may show, for example, at least 30%, at least 50%, at least 60%, at least 70%, at least 80%, at least 90%, at least 95% or even at least 98% or greater sequence identity over a certain defined length. The variant may result in “conservative” amino acid changes which do not affect structural and/or chemical properties. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons daring mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

In an alternative, variants of the polynucleotides of the present invention may be generated through recombinant methods. One possible method is a DNA shuffling technique such as MOLECULARBREEDING (Maxygen Inc., Santa Clara Calif.; described in U.S. Pat. No. 5,837,458; Chang, C.-C. et al. (1999) Nat Biotechnol. 17:793-797; Christians, F. C. et al. (1999) Nat Biotechnol. 17:259-264; and Crameri, A et al. (1996) Nat Biotechnol. 14:315-319) to alter or improve the biological properties of DITHP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

A “variant”^{t} of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

### THE INVENTION

In a particular embodiment, cDNA sequences derived from human tissues and cell lines were aligned based on nucleotide sequence identity and assembled into “consensus” or “template” sequences which are designated by the template identification numbers (template IDs) in column 2 of Table 1.

The sequence identification numbers (SEQ ID NO:s) corresponding to the template IDs are shown in column 1. The template sequences have similarity to GenBank sequences, or “hits,” as designated by the GI Numbers in column 3. The statistical probability of each GenBank hit is indicated by a probability score in column 4, and the functional annotation corresponding to each GenBank hit is listed in column 5.

The invention incorporates the nucleic acid sequences of these templates as disclosed in the Sequence Listing and the use of these sequences in the diagnosis and treatment of disease states characterized by defects in human molecules. The invention further utilizes these sequences in hybridization and amplification technologies, and in particular, in technologies which assess gene expression patterns correlated with specific cells or tissues and their responses in vivo or in vitro to pharmaceutical agents, toxins, and other treatments. In this manner, the sequences of the present invention are used to develop a transcript image for a particular cell or tissue.

Derivation of Nucleic Acid Sequences

cDNA was isolated from libraries constructed using RNA derived from normal and diseased human tissues and cell lines. The human tissues and cell lines used for cDNA library construction were selected from a broad range of sources to provide a diverse population of cDNAs representative of gene transcription throughout the human body. Descriptions of the human tissues and cell lines used for cDNA library construction are provided in the LIFESEQ database (Incyte Genomics, Inc. (Incyte), Palo Alto Calif.). Human tissues were broadly selected from, for example, cardiovascular, dermatologic, endocrine, gastrointestinal, hematopoietic/immune system, musculoskeletal, neural, reproductive, and urologic sources.

Cell lines used for cDNA library construction were derived from, for example, leukemic cells, teratocarcinomas, neuroepitheliomas, cervical carcinoma, lung fibroblasts, and endothelial cells. Such cell lines include, for example, THP-1, Jurkat, HUVEC, hNT2, WI38, HeLa, and other cell lines commonly used and available from public depositories (American Type Culture Collection, Manassas Va.). Prior to mRNA isolation, cell lines were untreated, treated with a pharmaceutical agent such as 5′-aza-2′-deoxycytidine, treated with an activating agent such as lipopolysaccharide in the case of leukocytic cell lines, or, in the case of endothelial cell lines, subjected to shear stress.

Sequencing of the cDNAs

Methods for DNA sequencing are well known in the art. Conventional enzymatic methods employ the Klenow fragment of DNA polymerase I, SEQUENASE DNA polymerase (U.S. Biochemical Corporation, Cleveland Ohio), Taq polymerase (Applied Biosystems, Foster City Calif.), thermostable 17 polymerase (Amersham Pharmacia Biotech, Inc. (Amersham Pharmacia Biotech), Piscataway N.J.), or combinations of polymerases and proofreading exonucleases such as those found in the ELONGASE amplification system (Life Technologies Inc. (Life Technologies), Gaithersburg Md.), to extend the nucleic acid sequence from an oligonucleotide primer annealed to the DNA template of interest Methods have been developed for the use of both single-stranded and double-stranded templates. Chain termination reaction products may be electrophoresed on urea-polyacrylamide gels and detected either by autoradiography (for radioisotope-labeled nucleotides) or by fluorescence (for fluorophore-labeled nucleotides). Automated methods for mechanized reaction preparation, sequencing, and analysis using fluorescence detection methods have been developed Machines used to prepare cDNAs for sequencing can include the MICROLAB 2200 liquid transfer system (Hamilton Company (Hamilton), Reno Nev.), Peltier thermal cycler (PTC200; MJ Research, Inc. (MJ Research), Watertown Mass.), and ABI CATALYST 800 thermal cycler (Applied Biosystems). Sequencing can be carried out using, for example, the ABI 373 or 377 (Applied Biosystems) or MEGABACE 1000 (Molecular Dynamics, Inc. (Molecular Dynamics), Sunnyvale Calif.) DNA sequencing systems, or other automated and manual sequencing systems well known in the art.

The nucleotide sequences of the Sequence Listing have been prepared by current, state-of-the-art, automated methods and, as such, may contain occasional sequencing errors or unidentified nucleotides. Such unidentified nucleotides are designated by an N. These infrequent unidentified bases do not represent a hindrance to practicing the invention for those skilled in the art. Several methods employing standard recombinant techniques may be used to correct errors and complete the missing sequence information. (See, e.g., those described in Ausubel, F. M. et al. (1997) Short Protocols in Molecular Bioloy, John Wiley & Sons, New York N.Y.; and Sambrook, J. et al. (1989) Molecular Cloning A Laboratory Manual, Cold Spring Harbor Press, Plainview N.Y.)

Assembly of cDNA Sequences

Human polynucleotide sequences may be assembled using programs or algorithms well known in the art. Sequences to be assembled are related, wholly or in part, and may be derived-from a single or many different transcripts. Assembly of the sequences can be performed using such programs as PHRAP (Phils Revised Assembly Program) and the GELVIEW fragment assembly system (GCG), or other methods known in the art.

Alternatively, cDNA sequences are used as “component” sequences that are assembled into “template” or “consensus” sequences as follows. Sequence chromatograms are processed, verified, and quality scores are obtained using PHRED. Raw sequences are edited using an editing pathway known as Block 1 (See, e.g., the LIFESEQ Assembled User Guide, Incyte Genomics, Palo Alto, Calif.). A series of BLAST comparisons is performed and low-information segments and repetitive elements (e.g., dinucleotide repeats, Alu repeats, etc.) are replaced by “n's”, or masked, to prevent spurious matches. Mitochondrial and ribosomal RNA sequences are also removed. The processed sequences are then loaded into a relational database management system (RDMS) which assigns edited sequences to existing templates, if available. When additional sequences are added into the RDMS, a process is initiated which modifies existing templates or creates new templates from works in progress (i.e., nonfinal assembled sequences) containing queued sequences or the sequences themselves. After the new sequences have been assigned to templates, the templates can be merged into bins. If multiple templates exist in one bin, the bin can be split and the templates reannotated.

Once gene bins have been generated based upon sequence alignments, bins are “clone joined” based upon clone information. Clone joining occurs when the 5′ sequence of one clone is present in one bin and the 3′ sequence from the same clone is present in a different bin, indicating that the two bins should be merged into a single bin. Only bins which share at least two different clones are merged.

A resultant template sequence may contain either a partial or a full length open reading frame, or all or part of a genetic regulatory element. This variation is due in part to the fact that the full length cDNAs of many genes are several hundred, and sometimes several thousand, bases in length. With current technology, cDNAs comprising the coding regions of large genes cannot be cloned because of vector limitations, incomplete reverse transcription of the mRNA, or incomplete “second strand” synthesis. Template sequences may be extended to include additional contiguous sequences derived from the parent RNA transcript using a variety of methods known to those of skill in the art. Extension may thus be used to achieve the full length coding sequence of a gene.

Analysis of the cDNA Sequences

The cDNA sequences are analyzed using a variety of programs and algorithms which are well known in the art. (See, e.g., Ausubel, 1997, sutra, Chapter 7.7; Meyers, R. A. (Ed.) (1995) Molecular Biology and Biotechnology, Wiley VCH, New York N.Y., pp. 856-853; and Table 7.) These analyses comprise both reading frame determinations, e.g., based on triplet codon periodicity for particular organisms (Fickett, J. W. (1982) Nucleic Acids Res. 10:5303-5318); analyses of potential start and stop codons; and homology searches.

Computer programs known to those of skill in the art for performing computer-assisted searches for amino acid and nucleic acid sequence similarity, include, for example, Basic Local Alignment Search Tool (BLAST; Altschul, S. F. (1993) J. Mol. Evol. 36:290-300; Altschul, S. F. et al. (1990) J. Mol. Biol. 215:403410). BLAST is especially useful in determining exact matches and comparing two sequence fragments of arbitrary but equal lengths, whose alignment is locally maximal and for which the alignment score meets or exceeds a threshold or cutoff score set by the user (Karlin, S. et al. (1988) Proc. Natl. Acad. Sci. USA 85:841-845). Using an appropriate search tool (e.g., BLAST or HMM), GenBank, SwissProt, BLOCKS, PFAM and other databases may be searched for sequences containing regions of homology to a query dithp or DITHP of the present invention.

Other approaches to the identification, assembly, storage, and display of nucleotide and polypeptide sequences are provided in “Relational Database for Storing Biomolecule Infomation,” U.S. Ser. No. 08/947,845, filed Oct. 9, 1997; “Project-Based Full-Length Biomolecular Sequence Database,” U.S. Ser. No. 08/811,758, filed Mar. 6, 1997; and “Relational Database and System for Storing Information Relating to Biomolecular Sequences,” U.S. Ser. No. 09/034,807, filed Mar. 4, 1998, all of which are incorporated by reference herein in their entirety.

Protein hierarchies can be assigned to the putative encoded polypeptide based on, e.g., motif, BLAST, or biological analysis. Methods for assigning these hierarchies are described, for example, in “Database System Employing Protein Function Hierarchies for Viewing Biomolecular Sequence Data,” U.S. Ser. No. 08/812,290, filed Mar. 6, 1997, incorporated herein by reference.

Identification of Human Diagnostic and Therapeutic Molecules Encoded by dithp

The identities of the DITHP encoded by the dithp of the present invention were obtained by analysis of the assembled cDNA sequences. SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, and SEQ ID NO:223, encoded by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, respectively, are, for example, human enzyme molecules.

SEQ ID NO:224, SEQ ID NO:225, SEQ ID NO:226, SEQ ID NO:227, and SEQ ID NO:228, encoded by SEQ ID NO:13, SEQ ID NO:14, SEQ ID NO:15, SEQ ID NO:16, and SEQ ID NO:17, respectively, are, for example, receptor molecules.

SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, and SEQ ID NO:241, encoded by SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30, respectively, are, for example, intracellular signaling molecules.

SEQ ID NO:242, SEQ ID NO:243, SEQ ID NO:244, SEQ ID NO:245, SEQ ID NO:246,
SEQ ID NO:247, SEQ ID NO:248, SEQ ID NO:249, SEQ ID NO:250, SEQ ID NO:251, SEQ ID
NO:252, SEQ ID NO:253, SEQ ID NO:254, SEQ ID NO:255, SEQ ID NO:256, SEQ ID NO:257,
SEQ ID NO:258, SEQ ID NO:259, SEQ ID NO:260, SEQ ID NO:261, SEQ ID NO:262, SEQ ID
NO:263, SEQ ID NO:264, SEQ ID NO:265, SEQ ID NO:266, SEQ ID NO:267, SEQ ID NO:268,
SEQ ID NO:269, SEQ ID NO:270, SEQ ID NO:271, SEQ ID NO:272, SEQ ID NO:273, SEQ ID
NO:274, SEQ ID NO:275, SEQ ID NO:276, SEQ ID NO:277, SEQ ID NO:278, SEQ ID NO:279,
SEQ ID NO:280, SEQ ID NO:281, SEQ ID NO:282, SEQ ID NO:283, SEQ ID NO:284, SEQ ID
NO:285, SEQ ID NO:286, SEQ ID NO:287, SEQ ID NO:288, SEQ ID NO:289, SEQ ID NO:290,
SEQ ID NO:291, SEQ ID NO:292, SEQ ID NO:293, SEQ ID NO:294, SEQ ID NO:295, SEQ ID
NO:296, SEQ ID NO:297, SEQ ID NO:298, SEQ ID NO:299, SEQ ID NO:300, SEQ ID NO:301,
SEQ ID NO:302, SEQ ID NO:303, SEQ ID NO:304, SEQ ID NO:305, SEQ ID NO:306, SEQ ID
NO:307, SEQ ID NO:308, and SEQ ID NO:309, encoded by SEQ ID NO:31, SEQ ID NO:32, SEQ
ID NO:33, SEQ ID NO:34, SEQ ID NO:35, SEQ ID NO:36, SEQ ID NO:37, SEQ ID NO:38, SEQ
ID NO:39, SEQ ID NO 40, SEQ ID NO:41, SEQ ID NO:42, SEQ ID NO:43, SEQ ID NO:44, SEQ
ID NO:45, SEQ ID NO:46, SEQ ID NO:47, SEQ ID NO:48, SEQ ID NO:49, SEQ ID NO:50, SEQ
ID NO:51, SEQ ID NO:52, SEQ ID NO:53, SEQ ID NO:54, SEQ ID NO:55, SEQ ID NO:56, SEQ
ID NO:57, SEQ ID NO:58, SEQ ID NO:59, SEQ ID NO:60, SEQ ID NO:61, SEQ ID NO:62, SEQ
ID NO:63, SEQ ID NO:64, SEQ ID NO:65, SEQ ID NO:66, SEQ ID NO:67, SEQ ID NO:68, SEQ
ID NO:69, SEQ ID NO:70, SEQ ID NO:71, SEQ ID NO:72, SEQ ID NO:73, SEQ ID NO:74, SEQ
ID NO:75, SEQ ID NO:76, SEQ ID NO:77, SEQ ID NO:78, SEQ ID NO:79, SEQ ID NO:S0, SEQ
ID NO:81, SEQ ID NO:82, SEQ ID NO:83, SEQ ID NO:84, SEQ ID NO:85, SEQ ID NO:86, SEQ
ID NO:87, SEQ ID NO:88, SEQ ID NO:89, SEQ ID NO:90, SEQ ID NO:91, SEQ ID NO:92, SEQ
ID NO:93, SEQ ID NO:94, SEQ ID NO:95, SEQ ID NO:96, SEQ ID NO:97, and SEQ ID NO:98,

respectively, are, for example, transcription factor molecules.

SEQ ID NO:310, SEQ ID NO:311, SEQ ID NO:312, SEQ ID NO:313, SEQ ID NO:314, SEQ ID NO:315, SEQ ID NO:316, and SEQ ID NO:317, encoded by SEQ ID NO:99, SEQ ID NO:100, SEQ ID NO:101, SEQ ID NO:102, SEQ ID NO:103, SEQ ID NO:104, SEQ ID NO:105, and SEQ ID NO:106, respectively, are, for example, membrane transport molecules.

SEQ ID NO:318, SEQ ID NO:319, SEQ ID NO:320, SEQ ID NO:321, SEQ ID NO:322,

SEQ ID NO:323, SEQ ID NO:324, SEQ ID NO:325, SEQ ID NO:326, and SEQ ID NO:327, encoded by SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, and SEQ ID NO:116, respectively, are, for example, protein modification and maintenance molecules.

SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, and SEQ ID NO:341, encoded by SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, and SEQ ID NO:130, respectively, are, for example, nucleic acid synthesis and modification molecules.

SEQ ID NO:342, encoded by SEQ ID NO:131 is, for example, an adhesion molecule. SEQ ID NO:343, SEQ ID NO:344, SEQ ID NO:345, SEQ ID NO:346, SEQ ID NO:347,

SEQ ID NO:348, and SEQ ID NO:349, encoded by SEQ ID NO:132, SEQ ID NO:133, SEQ ID NO:134, SEQ ID NO:135, SEQ ID NO:136, SEQ ID NO:137, and SEQ ID NO:138, respectively, are, for example, antigen recognition molecules.

SEQ ID NO:350, SEQ ID NO:351, SEQ ID NO:352, and SEQ ID NO:353, encoded by SEQ ID NO:139, SEQ ID NO:140, SEQ ID NO:141, and SEQ ID NO:142, respectively, are, for example, electron transfer associated molecules.

SEQ ID NO:354, SEQ ID NO:355, SEQ ID NO:356, SEQ ID NO:357, SEQ ID NO:358, and SEQ ID NO:359, encoded by SEQ ID NO:143, SEQ ID NO:144, SEQ ID NO:145, SEQ ID NO:146, SEQ ID NO:147, and SEQ ID NO:148, respectively, are, for example, secreted/extracellular matrix molecules.

SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, and SEQ ID NO:369, encoded by SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, and SEQ ID NO:158, respectively, are, for example, cytoskeletal molecules.

SEQ ID NO:370, SEQ ID NO:371, SEQ ID NO:372, and SEQ ID NO:373, encoded by SEQ ID NO:159, SEQ ID NO:160, SEQ ID NO:161, and SEQ ID NO:162, respectively, are, for example, cell membrane molecules.

SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, and SEQ ID NO:392, encoded by SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, and SEQ ID NO:181, respectively, are, for example, ribosomal molecules.

SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, and SEQ ID NO:403, encoded by SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, and SEQ ID NO:192, respectively, are, for example, organelle associated molecules.

SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO-0.412, SEQ ID NO:413, and SEQ ID NO:414, encoded by SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, and SEQ ID NO:203, respectively, are; for example, biochemical pathway molecules.

SEQ ID NO:415, SEQ ID NO:416, SEQ ID NO:417, SEQ ID NO:418, SEQ ID NO:419,

SEQ ID NO:420, SEQ ID NO:421, and SEQ ID NO:422, encoded by SEQ ID NO:204, SEQ ID NO:205, SEQ ID NO:206, SEQ ID NO:207, SEQ ID NO:208, SEQ ID NO:209, SEQ ID NO:210, and SEQ ID NO:211, respectively, are, for example, molecules associated with growth and development

Sequences of Hunan Diagnostic and Therapeutic Molecules

The dithp of the present invention may be used for a variety of diagnostic and therapeutic purposes. For example, a dithp may be used to diagnose a particular condition, disease, or disorder associated with human molecules. Such conditions, diseases, and disorders include, but are not limited to, a cell proliferative disorder, such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder, such as inflammation, actinic keratosis, acquired immunodeficiency syndrome (AIDS), Addison's disease, adult respiratory distress syndrome, allergies, ankylosing spondylitis, amyloidosis, anemia, arteriosclerosis, asthma, atherosclerosis, autoimmune hemolytic anemia, autoimmune thyroiditis, bronchitis, bursitis, cholecystitis, cirrhosis, contact dermatitis, Crohn's disease, atopic dermatitis, dermatomyositis, diabetes mellitus, emphysema, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinuria, hepatitis, hypereosinophilia, irritable bowel syndrome, episodic lymphopenia with lymphocytotoxins, mixed connective tissue disease (MCTD), multiple sclerosis, myasthenia gravis, myocardial or pericardial inflammation, myelofibrosis, osteoarthritis, osteoporosis, pancreatitis, polycythemia vera, polymyositis, psoriasis, Reiter's syndrome, rheumatoid arthritis, scleroderma, Sjögren's syndrome, systemic anaphylaxis, systemic lupus erythematosus, systemic sclerosis, primary thrombocythemia, thrombocytopenic purpura, ulcerative colitis, uveitis, Werner syndrome, complications of cancer, hemodialysis, and extracorporeal circulation, trauma, and hematopoietic cancer including lymphoma, leukemia, and myeloma; an infection caused by a viral agent classified as adenovinis, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fuingal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leisbmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestrode such as tapeworm; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma; a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism; a disorder associated with hyperpituitarism including acromegaly, giantism, and syndrome of inappropriate antidiuretic hormone (ADH) secretion (SIADH) often caused by benign adenoma; a disorder associated with hypothyroidism including goiter, myxedema, acute thyroiditis associated with bacterial infection, subacute thyroiditis associated with viral infection, autoimmune thyroiditis (Hashimoto's disease), and cretinism; a disorder associated with hyperthyroidism including thyrotoxicosis and its various forms, Grave's disease, pretibial myxedema, toxic multinodular goiter, thyroid carcinoma, and Plummer's disease; a disorder associated with hyperparathyroidism including Corn disease (chronic hypercalemia); a pancreatic disorder such as Type I or Type II diabetes mellitus and associated complications; a disorder associated with the adrenals such as hyperplasia, carcinoma, or adenoma of the adrenal cortex, hypertension associated with alkalosis, amyloidosis, hypokalemia, Gushing's disease, Liddle's syndrome, and Arnold-Healy-Gordon syndrome, pheochromocytoma tumors, and Addison's disease; a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia; a metabolic disorder such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, pentosuria phenylketonuria, pseudovitamin D-deficiency rickets; disorders of carbohydrate metabolism such as congenital type II dyseryhropoietic anemia, diabetes, insulin-dependent diabetes mellitus, non-insulin-dependent diabetes mellitus, fructose-1,6diphosphatase deficiency, galactosemia, glucagonoma, hereditary fructose intolerance, hypoglycemia, mannosidosis, neuraminidase deficiency, obesity, galactose epimerase deficiency, glycogen storage diseases, lysosomal storage diseases, fructosuria, pentosuria, and inherited abnormalities of pyruvate metabolism; disorders of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity; and disorders of copper metabolism such as Menke's disease, Wilson's disease, and Ehlers-Danlos syndrome type IX; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, and Tourette's disorder; a gastrointestinal disorder including ulcerative colitis, gastric and duodenal ulcers, cystinuria, dibasicaminoaciduria, hypercystinuria, lysinuria, hartnup disease, tryptophan malabsorption, methionine malabsorption, hissidinuria, iminoglycinuria, dicarboxylicaminoaciduria, cystinosis, renal glycosuria, hypouricemia, familial hypophophatemic rickets, congenital chloridorrhea, distal renal tubular acidosis, Menkes' disease, Wilson's disease, lethal diarrhea, juvenile pernicious anemia, folate malabsorption, adrenoleukodystrophy, hereditary myoglobinuria, and Zellweger syndrome; a transport disorder such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrymia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, and polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, cataracts, infertility, pulmonary artery stenosis, sensorineural autosomal deafness, hyperglycemia, hypoglycemia, Grave's disease, goiter, glucose-galactose malabsorption syndrome, hypercholesterolemia, Cushing's disease, and Addison's disease; and a connective tissue disorder such as osteogenesis imperfecta, Ehlers-Danlos syndrome, chondrodysplasias, Marfan syndrome, Alport syndrome, familial aortic aneurysm, achondroplasia, mucopolysaccharidoses, osteoporosis, osteopetrosis, Paget's disease, rickets, osteomalacia, hyperparathyroidism, renal osteodystrophy, osteonecrosis, osteomyelitis, osteoma, osteoid osteoma, osteoblastoma, osteosarcoma, osteochondroma, chondroma, chondroblastoma, chondromyxoid fibroma, chondrosarcoma, fibrous cortical defect, nonossifying fibroma, fibrous dysplasia, fibrosarcoma, malignant fibrous histiocytoma, Ewing's sarcoma, primitive neuroectodermal tumor, giant cell tumor, osteoarthritis, rheumatoid arthritis, ankylosing spondyloarthritis, Reiter's syndrome, psoriatic arthritis, enteropathic arthritis, infectious arthritis, gout, gouty arthritis, calcium pyrophosphate crystal deposition disease, ganglion, synovial cyst, villonodular synovitis, systemic sclerosis, Dupuytren's contracture, hepatic fibrosis, lupus erythematosus, mixed connective tissue disease, epidermolysis bullosa simplex, bullous congenital ichthyosiform erythroderma (epidermolytic hyperkeratosis), non-epidermolytic and epidermolytic palmoplantar keratoderma, ichthyosis bullosa of Siemens, pachyonycnia congenita, and white sponge nevus. The dithp can be used to detect the presence of, or to quantify the amount of, a dithp-related polynucleotide in a sample. This information is then compared to information obtained from appropriate reference samples, and a diagnosis is established. Alternatively, a polynucleotide complementary to a given dithp can inhibit or inactivate a therapeutically relevant gene related to the dithp.

Analysis of dithp Expression Patterns

The expression of dithp may be routinely assessed by hybridization-based methods to determine, for example, the tissue-specificity, disease-specificity, or developmental stage-specificity of dithp expression. For example, the level of expression of dithp may be compared among different cell types or tissues, among diseased and normal cell types or tissues, among cell types or tissues at different developmental stages, or among cell types or tissues undergoing various treatments. This type of analysis is useful, for example, to assess the relative levels of dithp expression in fully or partially differentiated cells or tissues, to determine if changes in dithp expression levels are correlated with the development or progression of specific disease states, and to assess the response of a cell or tissue to a specific therapy, for example, in pharmacological or toxicological studies. Methods for the analysis of dithp expression are based on hybridization and amplification technologies and include membrane-based procedures such as northern blot analysis, high-throughput procedures that utilize, for example, microarrays, and PCR-based procedures.

Hybridization and Genetic Analysis

The dithp, their fragments, or complementary sequences, may be used to identify the presence of and/or to determine the degree of similarity between two (or more) nucleic acid sequences. The dithp may be hybridized to naturally occurring or recombinant nucleic acid sequences under appropriately selected temperatures and salt concentrations. Hybridization with a probe based on the nucleic acid sequence of at least one of the dithp allows for the detection of nucleic acid sequences, including genomic sequences, which are identical or related to the dithp of the Sequence Listing. Probes may be selected from non-conserved or unique regions of at least one of the polynucleotides of SEQ ID NO:1-211 and tested for their ability to identity or amplify the target nucleic acid sequence using standard protocols.

Polynucleotide sequences that are capable of hybridizing, in particular, to those shown in SEQ ID NO:1-211 and fragments thereof, can be identified using various conditions of stringency. (See, e.g., Wahl, G. M. and S. L. Berger (1987) Methods Enzymol. 152:399-407; Kimmel, AR. (1987) Methods Enzymol. 152:507-511.) Hybridization conditions are discussed in “Definitions.”

A probe for use in Southern or northern hybridization may be derived from a fragment of a dithp sequence, or its complement, that is up to several hundred nucleotides in length and is either single-stranded or double-stranded. Such probes may be hybridized in solution to biological materials such as plasmids, bacterial, yeast, or human artificial chromosomes, cleared or sectioned tissues, or to artificial substrates containing dithp. Microarrays are particularly suitable for identifying the presence of and detecting the level of expression for multiple genes of interest by examining gene expression correlated with, e.g., various stages of development, treatment with a drug or compound, or disease progression. An array analogous to a dot or slot blot may be used to arrange and link polynucleotides to the surface of a substrate using one or more of the following: mechanical (vacuum), chemical, thermal, or UV bonding procedures. Such an array may contain any number of dithp and may be produced by hand or by using available devices, materials, and machines.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

Probes may be labeled by either PCR or enzymatic techniques using a variety of commercially available reporter molecules. For example, commercial kits are available for radioactive and chemiluminescent labeling (Amersham Pharmacia Biotech) and for alkaline phosphatase labeling (Life Technologies). Alternatively, dithp may be cloned into commercially available vectors for the production of RNA probes. Such probes may be transcribed in the presence of at least one labeled nucleotide (e.g., ³²P-ATP, Amersham Pharmacia Biotech).

Additionally the polynucleotides of SEQ ID NO:1-211 or suitable fragments thereof can be used to isolate full length cDNA sequences utilizing hybridization and/or amplification procedures well known in the art, e.g., cDNA library screening, PCR amplification, etc. The molecular cloning of such full length cDNA sequences may employ the method of cDNA library screening with probes using the hybridization, stringency, washing, and probing strategies described above and in Ausubel, supra, Chapters 3, 5, and 6. These procedures may also be employed with genomic libraries to isolate genomic sequences of dithp in order to analyze, e.g., regulatory elements.

Genetic Mapping

Gene identification and mapping are important in the investigation and treatment of almost all conditions, diseases, and disorders. Cancer, cardiovascular disease, Alzheimer's disease, arthritis, diabetes, and mental illnesses are of particular interest. Each of these conditions is more complex than the single gene defects of sickle cell anemia or cystic fibrosis, with select groups of genes being predictive of predisposition for a particular condition, disease, or disorder. For example, cardiovascular disease may result from malfunctioning receptor molecules that fail to clear cholesterol from the bloodstream, and diabetes may result when a particular individual's immune system is activated by an infection and attacks the insulin-producing cells of the pancreas. In some studies, Alzieimer's disease has been linked to a gene on chromosome 21; other studies predict a different gene and location. Mapping of disease genes is a complex and reiterative process and generally proceeds from genetic linkage analysis to physical mapping.

As a condition is noted among members of a family, a genetic linkage map traces parts of chromosomes that are inherited in the same pattern as the condition. Statistics link the inheritance of particular conditions to particular regions of chromosomes, as defined by RFLP or other markers. (See, for example, Lander, E. S. and Botstein, D. (1986) Proc. Natl. Acad. Sci. USA 83:7353-7357.) Occasionally, genetic markers and their locations are known from previous studies. More often, however, the markers are simply stretches of DNA that differ among individuals. Examples of genetic linkage maps can be found in various scientific journals or at the Online Mendelian Inheritance in Man (OMIM) World Wide Web site.

In another embodiment of the invention, dithp sequences may be used to generate hybridization probes useful in chromosomal mapping of naturally occurring genomic sequences. Either coding or noncoding sequences of dithp may be used, and in some instances, noncoding sequences may be preferable over coding sequences. For example, conservation of a dithp coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat Genet. 15:345-355; Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Meyers, supra, pp. 965-968.) Correlation between the location of dithp on a physical chromosomal map and a specific disorder, or a predisposition to a specific disorder, may help define the region of DNA associated with that disorder. The dithp sequences may also be used to detect polymorphisms that are genetically linked to the inheritance of a particular condition, disease, or disorder.

In situ hybridization of chromosomal preparations and genetic mapping techniques, such as linkage analysis using established chromosomal markers, may be used for extending existing genetic maps. Often the placement of a gene on the chromosome of another mammalian species, such as mouse, may reveal associated markers even if the number or arm of the corresponding human chromosome is not known. These new marker sequences can be mapped to human chromosomes and may provide valuable information to investigators searching for disease genes using positional cloning or other gene discovery techniques. Once a disease or syndrome has been crudely correlated by genetic linkage with a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequences of the subject invention may also be used to detect differences in chromosomal architecture due to translocation, inversion, etc., among normal, carrier, or affected individuals.

Once a disease-associated gene is mapped to a chromosomal region, the gene must be cloned in order to identify mutations or other alterations (e.g., translocations or inversions) that may be correlated with disease. This process requires a physical map of the chromosomal region containing the disease-gene of interest along with associated markers. A physical map is necessary for determining the nucleotide sequence of and order of marker genes on a particular chromosomal region. Physical mapping techniques are well known in the art and require the generation of overlapping sets of cloned DNA fragments from a particular organelle, chromosome, or genome. These clones are analyzed to reconstruct and catalog their order. Once the position of a marker is determined, the DNA from that region is obtained by consulting the catalog and selecting clones from that region. The gene of interest is located through positional cloning techniques using hybridization or similar methods.

Diagnostic Uses

The dithp of the present invention may be used to design probes useful in diagnostic assays. Such assays, well known to those skilled in the art, may be used to detect or confirm conditions, disorders, or diseases associated with abnormal levels of dithp expression. Labeled probes developed from dithp sequences are added to a sample under hybridizing conditions of desired stringency. In some instances, dithp, or fragments or oligonucleotides derived from dithp, may be used as primers in amplification steps prior to hybridization. The amount of hybridization complex formed is quantified and compared with standards for that cell or tissue. If dithp expression varies significantly from the standard, the assay indicates the presence of the condition, disorder, or disease. Qualitative or quantitative diagnostic methods may include northern, dot blot, or other membrane or dip-stick based technologies or multiple-sample format technologies such as PCR, enzyme-linked immunosorbent assay (ELISA)-like, pin, or chip-based assays.

The probes described above may also be used to monitor the progress of conditions, disorders, or diseases associated with abnormal levels of dithp expression, or to evaluate the efficacy of a particular therapeutic treatment. The candidate probe may be identified from the dithp that are specific to a given human tissue and have not been observed in GenBank or other genome databases. Such a probe may be used in animal studies, preclinical tests, clinical trials, or in monitoring the treatment of an individual patient. In a typical process, standard expression is established by methods well known in the art for use as a basis of comparison, samples from patients affected by the disorder or disease are combined with the probe to evaluate any deviation from the standard profile, and a therapeutic agent is administered and effects are monitored to generate a treatment profile. Efficacy is evaluated by determining whether the expression progresses toward or returns to the standard normal pattern. Treatment profiles may be generated over a period of several days or several months. Statistical methods well known to those skilled in the art may be use to determine the significance of such therapeutic agents.

The polynucleotides are also useful for identifying individuals from minute biological samples, for example, by matching the RFLP pattern of a sample's DNA to that of an individual's DNA The polynucleotides of the present invention can also be used to determine the actual base-by-base DNA sequence of selected portions of an individual's genome. These sequences can be used to prepare PCR primers for amplifying and isolating such selected DNA, which can ten be sequenced. Using this technique, an individual can be identified through a unique set of DNA sequences. Once a unique ID database is established for an individual, positive identification of that individual can be made from extremely small tissue samples.

In a particular aspect, oligonucleotide primers derived from the dithp of the invention may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from dithp are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequences of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

DNA-based identification techniques are critical in forensic technology. DNA sequences taken from very small biological samples such as tissues, e.g., hair or skin, or body fluids, e.g., blood, saliva, semen, etc., can be amplified using, e.g., PCR, to identify individuals. (See, e.g., Erlich, H. (1992) PCR Technology, Freeman and Co., New York N.Y.). Similarly, polynucleotides of the present invention can be used as polymorphic markers.

There is also a need for reagents capable of identifying the source of a particular tissue. Appropriate reagents can comprise, for example, DNA probes or primers prepared from the sequences of the present invention that are specific for particular tissues. Panels of such reagents can identify tissue by species and/or by organ type. In a similar fashion, these reagents can be used to screen tissue cultures for contamination.

The polynucleotides of the present invention can also be used as molecular weight markers on nucleic acid gels or Southern blots, as diagnostic probes for the presence of a specific mRNA in a particular cell type, in the creation of subtracted cDNA libraries which aid in the discovery of novel polynucleotides, in selection and synthesis of oligomers for attachment to an array or other support, and as an antigen to elicit an immune response.

Disease Model Systems Using dithp

The dithp of the invention or their mammalian homologs may be “knocked out” in an animal model system using homologous recombination in embryonic stem (ES) cells. Such techniques are well known in the art and are useful for the generation of animal models of human disease. (See, e.g., U.S. Pat. Nos. 5,175,383 and 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

The dithp of the invention may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

The dithp of the invention can also be used to create “knockin” humanized animals (pigs) or transgenic animals (mice or rats) to model human disease. With knockin technology, a region of dithp is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress dithp, resulting, e.g., in the secretion of DITHP in its milk, may also serve as a convenient source of that protein (Janne, J. et al. (1998) Biotechnol. Annu. Rev. 4:55-74).

Screening Assays

DITHP encoded by polynucleotides of the present invention may be used to screen for molecules that bind to or are bound by the encoded polypeptides. The binding of the polypeptide and the molecule may activate (agonist), increase, inhibit (antagonist), or decrease activity of the polypeptide or the bound molecule. Examples of such molecules include antibodies, oligonucleotides, proteins (e.g., receptors), or small molecules.

Preferably, the molecule is closely related to the natural ligand of the polypeptide, e.g., a ligand or fragment thereof, a natural substrate, or a structural or functional mimetic. (See, Coligan et al., (1991) Current Protocols in Immunology 1(2): Chapter 5.) Similarly, the molecule can be closely related to the natural receptor to which the polypeptide binds, or to at least a fragment of the receptor, e.g., the active site. In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide or cell membrane fractions which contain the expressed polypeptide are then contacted with a test compound and binding, stimulation, or inhibition of activity of either the polypeptide or the molecule is analyzed.

An assay may simply test binding of a candidate compound to the polypeptide, wherein binding is detected by a fluorophore, radioisotope, enzyme conjugate, or other detectable label. Alternatively, the assay may assess binding in the presence of a labeled competitor.

Additionally, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay using, e.g., a monoclonal or polyclonal antibody, can measure polypeptide level in a sample. The antibody can measure polypeptide level by either binding, directly or indirectly, to the polypeptide or by competing with the polypeptide for a substrate.

All of the above assays can be used in a diagnostic or prognostic context. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues.

Transcript Imaging and Toxicological Testing

Another embodiment relates to the use of dithp to develop a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity pertaining to human molecules for diagnostics and therapeutics.

Transcript images which profile dithp expression may be generated using transcripts isolated from tissues, cell lines, biopsies, or other biological samples. The transcript image may thus reflect dithp expression in vivo, as in the case of a tissue or biopsy sample, or in vitro, as in the case of a cell line.

Transcript images which profile dithp expression may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and Anderson, N. L. (2000) Toxicol. Lett. 112-113:467-71, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalized the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http:/www.niehs.nih.gov/oc./news/toxchip.htm). Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of DITHP encoded by polynucleotides of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for DITHP to quantify the levels of DITHP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-11; Mendoze, L. G. et al. (1999) Biotechniques 27:778-88). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and Seilhamer, J. (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the DITHP encoded by polynucleotides of the present invention.

In another embodiment, the toxicity of a test compound is assessed by treating a biological sample containing proteins with the test compound. Proteins from the biological sample are incubated with antibodies specific to the DITHP encoded by polynucleotides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Transcript images may be used to profile dithp expression in distinct tissue types. This process can be used to determine human molecule activity in a particular tissue type relative to this activity in a different tissue type. Transcript images may be used to generate a profile of dithp expression characteristic of diseased tissue. Transcript images of tissues before and after treatment may be used for diagnostic purposes, to monitor the progression of disease, and to monitor the efficacy of drug treatments for diseases which affect the activity of human molecules.

Transcript images of cell lines can be used to assess human molecule activity and/or to identity cell lines that lack or misregulate this activity. Such cell lines may then be treated with pharmaceutical agents, and a transcript image following treatment may indicate the efficacy of these agents in restoring desired levels of this activity. A similar approach may be used to assess the toxicity of pharmaceutical agents as reflected by undesirable changes in human molecule activity. Candidate pharmaceutical agents may be evaluated by comparing their associated transcript images with those of pharmaceutical agents of known effectiveness.

Antisense Molecules

The polynucleotides of the present invention are useful in antisense technology. Antisense technology or therapy relies on the modulation of expression of a target protein through the specific binding of an antisense sequence to a target sequence encoding the target protein or directing its expression. (See, e.g., Agrawal, S., ed. (1996) Antisense Therapeutics, Humana Press Inc., Totawa N.J.; Alama, A. et al. (1997) Pharmacol. Res. 36(3):171-178; Crooke, S. T. (1997) Adv. Pharmacol. 40:149; Sharma, H. W. and R. Narayanan (1995) Bioessays 17(12):1055-1063; and Lavrosky, Y. et al. (1997) Biochem. Mol. Med. 62(1):11-22.) An antisense sequence is a polynucleotide sequence capable of specifically hybridizing to at least a portion of the target sequence. Antisense sequences bind to cellular mRNA and/or genomic DNA, affecting translation and/or transcription. Antisense sequences can be DNA, RNA, or nucleic acid mimics and analogs. (See, e.g., Rossi, J. J. et al (1991) Antisense Res. Dev. 1(3):285-288; Lee, R. et al. (1998) Biochemistry 37(3):900-1010; Pardridge, W. M. et al. (1995) Proc. Natl. Acad. Sci. USA 92(12):5592-5596; and Nielsen, P. E. and Haaima, G. (1997) Chem Soc. Rev. 96:73-78.) Typically, the binding which results in modulation of expression occurs through hybridization or binding of complementary base pairs. Antisense sequences can also bind to DNA duplexes through specific interactions in the major groove of the double helix.

The polynucleotides of the present invention and fragments thereof can be used as antisense sequences to modify the expression of the polypeptide encoded by dithp. The antisense sequences can be produced ex vivo, such as by using any of the ABI nucleic acid synthesizer series (Applied Biosystems) or other automated systems known in the art. Antisense sequences can also be produced biologically, such as by transforming an appropriate host cell with an expression vector containing the sequence of interest. (See, e.g., Agrawal, supra.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E., et al. (1998) J. Allergy Clin. Immunol. 102(3):469-475; and Scanlon, K. J., et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel F. M. et al. (1995) Current Protocols in Molecular Biology, John Wiley & Sons, New York N.Y.; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci 87(11):1308-1315; and Morris, M. C. et al. (1997) Nucleic Acids Res. 25(14):2730-2736.)

Expression

In order to express a biologically active DITHP, the nucleotide sequences encoding DITHP or fragments thereof may be inserted into an appropriate expression vector, i.e., a vector which contains the necessary elements for transcriptional and translational control of the inserted coding sequence in a suitable host. Methods which are well known to those skilled in the art may be used to construct expression vectors containing sequences encoding DITHP and appropriate transcriptional and translational control elements. These methods include in vitro recombinant DNA techniques, synthetic techniques, and in vivo genetic recombination. (See, e.g., Sambrook, supra, Chapters 4, 8, 16, and 17; and Ausubel, supra, Chapters 9, 10, 13, and 16.)

A variety of expression vector/host systems may be utilized to contain and express sequences encoding DITHP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal (mammalian) cell systems. (See, e.g., Sambrook, supra; Ausubel, 1995, supra, Van Heeke, G. and S. M. Schuster (1989) J. Biol. Chem 264:5503-5509; Bitter, G. A. et al. (1987) Methods Enzymol. 153:516-544; Scorer, C. A et al. (1994) Bio/Technology 12:181-184; Engelhard, E. K. et al. (1994) Proc. Natl. Acad. Sci. USA 91:3224-3227; Sandig, V. et al. (1996) Hume Gene Ther. 7:1937-1945; Takamatsu, N. (1987) EMBO J. 6:307-311; Coruzzi, G. et al. (1984) EMBO J. 3:1671-1680; Broglie, R. et al. (1984) Science 224:838-843; Winter, J. et al. (1991) Results Probl. Cell Differ. 17:85-105; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al., (1993) Proc. Nail. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

For long term production of recombinant proteins in mammalian systems, stable expression of DITHP in cell lines is preferred. For example, sequences encoding DITHP can be transformed into cell lines using expression vectors which may contain viral origins of replication and/or endogenous expression elements and a selectable marker gene on the same or on a separate vector. Any number of selection systems may be used to recover transformed cell lines. (See, e.g., Wigler, M. et al. (1977) Cell 11:223-232; Lowy, I. et al. (1980) Cell 22:817-823.; Wigler, M. et al. (1980) Proc. Natl. Acad. Sci. USA 77:3567-3570; Colbere-Garapin, F. et al. (1981) J. Mol. Biol. 150:1-14; Hartman, S. C. and R. C. Mulligan (1988) Proc. Natl. Acad. Sci. USA 85:8047-8051; Rhodes, C. A. (1995) Methods Mol. Biol. 55:121-131.)

Therapeutic Uses of dithp

The dithp of the invention may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hun Gene Therapy 6:643-666; Crystal, R. G. et al. (1995) Hum. Gene Therapy 6:667-703), thalassemias, familial hypercholesterolemia, and hemophilia resulting from Factor VIII or Factor IX deficiencies (Crystal, R. G. (1995) Science 270:404-410; Verma, I. M. and Somia, N. (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (IV) (Baltimore, D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in dithp expression or regulation causes disease, the expression of dithp from an appropriate population of transduced cells may alleviate the clinical manifestations caused by the genetic deficiency.

In a further embodiment of the invention, diseases or disorders caused by deficiencies in dithp are treated by constructing mammalian expression vectors comprising dithp and introducing these vectors by mechanical means into dithp deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and Anderson, W. F. (1993) Annu. Rev. Biochem 62:191-217; Ivics, Z. (1997) Cell 91:501-510; Boulay, J. -L. and Recipon, H. (1998) Curr. Opin. Biotechnol. 9:445-450).

Expression vectors that may be effective for the expression of dithp include, but are not limited to, the PCDNA 3.1, EPITAG, PRCCMV2, PREP, PVAX vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). The dithp of the invention may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and Bujard, H. (1992) Proc. Natl. Acad. Sci. U.S.A. 89:5547-5551; Gossen, M. et al., (1995) Science 268:1766-1769; Rossi, F. M. V. and Blau, H. N. (1998) Curr. Opin. Biotechnol. 9:451-456), commercially available in the T-REX plasmid (Invitrogen); the ecdysone-inducible promoter (available in the plasmids PVGRXR and PIND; Invitrogen); the FK506/rapamycin inducible promoter; or the RU486/mifepristone inducible promoter (Rossi, F. M. V. and Blau, H. M. supra), or (iii) a tissue-specific promoter or the native promoter of the endogenous gene encoding DITHP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and Eb, A. J. (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to dithp expression are treated by constructing a retrovirus vector consisting of (i) dithp under the control of an independent promoter or the retrovirus long terminal repeat (LTR) promoter, (ii) appropriate RNA packaging signals, and (ii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. U.S.A. 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and Miller, A. D. (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et ,al. (1998) Proc. Natl. Acad. Sci. U.S.A. 95:1201-1206; Su, L. (1997) Blood 89:2283-2290).

In the alternative, an adenovirus-based gene therapy delivery system is used to deliver dithp to cells which have one or more genetic abnormalities with respect to the expression of dithp. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and Somia, N. (1997) Nature 18:389:239-242, both incorporated by reference herein.

In another alternative, a herpes-based, gene therapy delivery system is used to deliver dithp to target cells which have one or more genetic abnormalities with respect to the expression of dithp. The use of herpes simplex virus (HSV)-based vectors may be especially valuable for introducing dithp to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res.169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. 1999 J. Virol. 73:519-532 and Xu, H. et al., (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver dithp to target cells. The biology of the prototypic alphavirus, Semliki Forest Virus (SFV), has been studied extensively and gene transfer vectors have been based on the SFV genome (Garoff, H. and Li, K. -J. (1998) Curr. Opin. Biotech. 9:464-469). During alphavirus RNA replication, a subgenomic RNA is generated that normally encodes the viral capsid proteins. This subgenomic RNA replicates to higher levels than the full-length genomic RNA, resulting in the overproduction of capsid proteins relative to the viral proteins with enzymatic activity (e.g., protease and polymerase). Similarly, inserting dithp into the alphavirus genome in place of the capsid-coding region results in the production of a large number of dithp RNAs and the synthesis of high levels of DITHP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:7483). The wide host range of alphaviruses will allow the introduction of dithp into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skin in the art.

Antibodies

Anti-DITHP antibodies may be used to analyze protein expression levels. Such antibodies include, but are not limited to, polyclonal, monoclonal, chimeric, single chain, and Fab fragments. For descriptions of and protocols of antibody technologies, see, e.g., Pound J. D. (1998) Immunochemical Protocols, Humana Press, Totowa, N.J.

The amino acid sequence encoded by the dithp of the Sequence Listing may be analyzed by appropriate software (e.g., LASERGENE NAVIGATOR software, DNASTAR) to determine regions of high immunogenicity. The optimal sequences for immunization are selected from the C-terminus, the N-terminus, and those intervening, hydrophilic regions of the polypeptide which are likely to be exposed to the external environment when the polypeptide is in its natural conformation. Analysis used to select appropriate epitopes is also described by Ausubel (1997, supra, Chapter 11.7). Peptides used for antibody induction do not need to have biological activity; however, they must be antigenic. Peptides used to induce specific antibodies may have an amino acid sequence consisting of at least five amino acids, preferably at least 10 amino acids, and most preferably at least 15 amino acids. A peptide which mimics an antigenic fragment of the natural polypeptide may be fused with another protein such as keyhole limpet hemocyanin (KLH; Sigma, St. Louis Mo.) for antibody production. A peptide encompassing an antigenic region may be expressed from a dithp, synthesized as described above, or purified from human cells.

Procedures well known in the art may be used for the production of antibodies. Various hosts including mice, goats, and rabbits, may be immunized by injection with a peptide. Depending on the host species, various adjuvants may be used to increase immunological response.

In one procedure, peptides about 15 residues in length may be synthesized using an ABI 431A peptide synthesizer (Applied Biosystems) using fmoc-chemistry and coupled to KLH (Sigma) by reaction with M-maleimidobenzoyl-N-hydroxysuccinimide ester (Ausubel, 1995, supra). Rabbits are immunized with the peptide-KLH complex in complete Freund's adjuvant. The resulting antisera are tested for antipeptide activity by binding the peptide to plastic, blocking with 1% bovine serum albumin (BSA), reacting with rabbit antisera, washing, and reacting with radioiodinated goat anti-rabbit IgG. Antisera with antipeptide activity are tested for anti-DITHP activity using protocols well known in the art, including ELISA, radioimmunoassay (RIA), and immunoblotting.

In another procedure, isolated and purified peptide may be used to immunize mice (about 100 μg of peptide) or rabbits (about 1 mg of peptide). Subsequently, the peptide is radioiodinated and used to screen the immunized animals' B-lymphocytes for production of antipeptide antibodies. Positive cells are then used to produce hybridomas using standard techniques. About 20 mg of peptide is sufficient for labeling and screening several thousand clones. Hybridomas of interest are detected by screening with radioiodinated peptide to identify those fusions producing peptide-specific monoclonal antibody. In a typical protocol, wells of a multi-well plate (FAST, Becton-Dickinson, Palo Alto, Calif.) are coated with affinty-purified, specific rabbit-anti-mouse (or suitable anti-species IgG) antibodies at 10 mg/ml. The coated wells are blocked with 1% BSA and washed and exposed to supernatants from hybridomas. After incubation, the wells are exposed to radiolabeled peptide at 1 mg/ml.

Clones producing antibodies bind a quantity of labeled peptide that is detectable above background. Such clones are expanded and subjected to 2 cycles of cloning. Cloned hybridomas are injected into pristane-treated mice to produce ascites, and monoclonal antibody is purified from the ascitic fluid by affinity chromatography on protein A (Amersham Pharmacia Biotech). Several procedures for the production of monoclonal antibodies, including in vitro production, are described in Pound (supra). Monoclonal antibodies with antipeptide activity are tested for anti-DITHP activity using protocols well known in the art, including ELISA, RIA, and immunoblotting.

Antibody fragments containing specific binding sites for an epitope may also be generated. For example, such fragments include, but are not limited to, the F(ab′)₂ fragments produced by pepsin digestion of the antibody molecule, and the Fab fragments generated by reducing the disulfide bridges of the F(ab′)₂ fragments. Alternatively, construction of Fab expression libraries in filamentous bacteriophage allows rapid and easy identification of monoclonal fragments with desired specificity (Pound, supra, Chaps. 45-47). Antibodies generated against polypeptide encoded by dithp can be used to purify and characterize full-length DITHP protein and its activity, binding partners, etc.

Assays Using Antibodies

Anti-DITHP antibodies may be used in assays to quantify the amount of DRIP found in a particular human cell. Such assays include methods utilizing the antibody and a label to detect expression level under normal or disease conditions. The peptides and antibodies of the invention may be used with or without modification or labeled by joining them, either covalently or noncovalently, with a reporter molecule.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the DITHP and its specific antibody and the measurement of such complexes. These and other assays are described in Pound (supra).

Without ft elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/184,777, U.S. Ser. No. 60/184,797, U.S. Ser. No. 60/184,698, U.S. Ser. No. 60/184,770, U.S. Ser. No. 60/184,774, U.S. Ser. No. 60/184,693, U.S. Ser. No. 60/184,771,U.S. Ser. No. 60/184,813, U.S. Ser. No. 60/184,773, U.S. Ser. No. 60/184,776, U.S. Ser. No. 60/184,769, U.S. Ser. No. 60/184,768, U.S. Ser. No. 60/184,837, U.S. Ser. No. 60/184,697, U.S. Ser. No. 60/184,841, U.S. Ser. No. 60/184,772, U.S. Ser. No. 60/185,213, U.S. Ser. No. 60/185,216, U.S. Ser. No. 60/204,863, U.S. Ser. No. 60/205,221, U.S. Ser. No. 60/204,815, U.S. Ser. No. 60/203,785, U.S. Ser. No. 60/204,821, U.S. Ser. No. 60/204,908, U.S. Ser. No. 60/204,226, U.S. Ser. No. 60/204,525, U.S. Ser. No. 60/205,285, U.S. Ser. No. 60/205,232, U.S. Ser. No. 60/205,323, U.S. Ser. No. 60/205,287, U.S. Ser. No. 60/205,324, and U.S. Ser. No. 60/205,286, are hereby expressly incorporated by reference.

### EXAMPLES

I. Construction of cDNA Libraries

RNA was purchased from CLONTECH Laboratories, Inc. Palo Alto Calif.) or isolated from various tissues. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated with either isopropanol or sodium acetate and ethanol, or by other routine methods.

Phenol extraction and precipitation of RNA were repeated as necessary to increase RNA purity. In most cases, RNA was treated with DNase. For most libraries, poly(A+) RNA was isolated using oligo dm-coupled paramagnetic particles (Promega Corporation (Promega), Madison Wis.), OLIGOTEX latex particles (QIAGEN, Inc. (QIAGEN), Valencia Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Inc., Austin IX).

In some cases, Stratagene was provided with RNA and constructed the corresponding cDNA libraries. Otherwise, cDNA was synthesized and cDNA libraries were constructed with the UNIZAP vector system (Stratagene Cloning Systems, Inc. (Stratagene), La Jolla Calif.) or SUPERSCRIPT plasmid system (Life Technologies), using the recommended procedures or similar methods known in the art. (See, e.g., Ausubel, 1997, supra, Chapters 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHAROSE S1000, SEPHAROSE CL2B, or SEPHAROSE C14B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid nitrogen, Carlsbad Calif.), PBK-CMV plasmid (Stratagene), or pINCY (Incyte Genomics, Palo Alto Calif.), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids were recovered from host cells by in vivo excision using the UNIZAP vector system (Stratagene) or by cell lysis. Plasmids were purified using at least one of the following: the Magic or WIZARD Minipreps DNA purification system (Promega); the AGTC Miniprep purification kit (Edge BioSystems, Gaithersburg Md.); and the QIAWELL 8, QIAWELL 8 Plus, and QIAWELL 8 Ultra plasmid purification systems or the R.E.A.L. PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format. (Rao, V. B. (1994) Anal. Biochem. 216:1-14.) Host cell lysis and thermal cycling steps were carried out in a single reaction mixture. Samples were processed and stored in 384 well plates, and the concentration of amplified plasmid DNA was quantified fluorometrically using PICOGREEN dye (Molecular Probes, Inc. (Molecular Probes), Eugene Oreg.) and a FLUOROSKAN II fluorescence scanner (Labsystems Oy, Helsinki, Finland).

III. Sequencing and Analysis

cDNA sequencing reactions were processed using standard methods or high-throughput instrumentation such as the ABI CATALYST 800 thermal cycler (Applied Biosystems) or the PTC-200 thermal cycler (MJ Research) in conjunction with the HYDRA microdispenser (Robbins Scientific Corp., Sunnyvale Calif.) or the MICROLAB 2200 liquid transfer system (Hamilton). cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art Reading frames within the cDNA sequences were identified using standard methods (reviewed in Ausubel, 1997, supra, Chapter 7.7). Some of the cDNA sequences were selected for extension using the techniques disclosed in Example VIII.

IV. Assembly and Analysis of Sequences

Component sequences from chromatograms were subject to PHRED analysis and assigned a quality score. The sequences having at least a required quality score were subject to various pre-processing editing pathways to eliminate, e.g., low quality 3′ ends, vector and linker sequences, polyA tails, Alu repeats, mitochondrial and ribosomal sequences, bacterial contamination sequences, and sequences smaller than 50 base pairs. In particular, low-information sequences and repetitive elements (e.g., dinucleotide repeats, Alu repeats, etc.) were replaced by “n's”, or masked, to prevent spurious matches.

Processed sequences were then subject to assembly procures in which the sequences were assigned to gene bins (ins). Each sequence could only belong to one bin. Sequences in each gene bin were assembled to produce consensus sequences (templates). Subsequent new sequences were added to existing bins using BLASTn (v. 1.4 WashU) and CROSSMATCH. Candidate pairs were identified as all BLAST hits having a quality score greater than or equal to 150. Alignments of at least 82% local identity were accepted into the bin. The component sequences from each bin were assembled using a version of PHRAP. Bins with several overlapping component sequences were assembled using DEEP PHRAP. The orientation (sense or antisense) of each assembled template was determined based on the number and orientation of its component sequences. Template sequences as disclosed in the sequence listing correspond to sense strand sequences (the “forward” reading frames), to the best determination. The complementary (antisense) strands are inherently disclosed herein The component sequences which were used to assemble each template consensus sequence are listed in Table 4, along with their positions along the template nucleotide sequences.

Bins were compared against each other and those having local similarity of at least 82% were combined and reassembled. Reassembled bins having templates of insufficient overlap (less than 95% local identity) were re-split. Assembled templates were also subject to analysis by STITCHER/EXON MAPPER algorithms which analyze the probabilities of the presence of splice variants, alternatively spliced exons, splice junctions, differential expression of alternative spliced genes across tissue types or disease states, etc. These resulting bins were subject to several rounds of the above assembly procedures.

Once gene bins were generated based upon sequence alignments, bins were clone joined based upon clone information. If the 5′ sequence of one clone was present in one bin and the 3′ sequence from the same clone was present in a different bin, it was likely that the two bins actually belonged together in a single bin. The resulting combined bins underwent assembly procedures to regenerate the consensus sequences.

The final assembled templates were subsequently annotated using the following procedure. Template sequences were analyzed using BLASTn (v2.0, NCBI) versus gbpri (GenBank version 120). “Hits” were defined as an exact match having from 95% local identity over 200 base pairs through 100% local identity over 100 base pairs, or a homolog match having an E-value, i.e. a probability score, of ≦1×10⁻⁸. The hits were subject to frameshift FASTx versus GENPEPT (GenBank version 120). (See Table 7). In this analysis, a homolog match was defined as having an E-value of ≦1×10⁻⁸. The assembly method used above was described in “System and Methods for Analyzing Biomolecular Sequences,” U.S. Ser. No. 09/276,534, filed Mar. 25, 1999, and the LIFESEQ Gold user manual (Incyte) both incorporated by reference herein.

Following assembly, template sequences were subjected to motif, BLAST, and functional analyses, and categorized in protein hierarchies using methods described in, e.g., “Database System Employing Protein Function Hierarchies for Viewing Biomolecular Sequence Data,” U.S. Ser. No. 08/812,290, filed Mar. 6, 1997; “Relational Database for Storing Biomolecule Information,” U.S. Ser. No. 08/947,845, filed Oct. 9, 1997; “Project-Based Full-Length Biomolecular Sequence Database,” U.S. Ser. No. 08/811,758, filed Mar. 6, 1997; and “Relational Database and System for Storing Information Relating to Biomolecular Sequences,” U.S. Ser. No. 09/034,807, filed Mar. 4, 1998, all of which are incorporated by reference herein.

The template sequences were further analyzed by translating each template in all three forward reading frames and searching each translation against the Pfam database of hidden Markov model-based protein families and domains using the HOMER software package (available to the public from Washington University School of Medicine, St. Louis Mo.). Regions of templates which, when translated, contain similarity to Pfam consensus sequences are reported in Table 2, along with descriptions of Pfam protein domains and families. Only those Pfam hits with an E-value of ≦1×10⁻³ are reds (See also World Wide Web site http:/pfam.wustl.edu/for detailed descriptions of Pfam protein domains and families.)

Additionally, the template sequences were translated in all three forward reading frames, and each translation was searched against hidden Markov models for signal peptides using the HMMER software package. Construction of hidden Markov models and their usage in sequence analysis has been described. (See, for example, Eddy, S. R. (1996) Curr. Opin. Str. Biol. 6:361-365.) Oly those signal peptide hits with a cutoff score of 11 bits or greater are reported. A cutoff score of 11 bits or greater corresponds to at least about 91-94% true-positives in signal peptide prediction. Template sequences were also translated in all three forward reading frames, and each translation was searched against TMAP, a program that uses weight matrices to delineate transmembrane segments on protein sequences and determine orientation, with respect to the cell cytosol (Persson, B. and P. Argos (1994) J. Mol. Biol. 237:182-192; Persson, B. and P. Argos (1996) Protein Sci. 5:363-371). Regions of templates which, when translated, contain similarity to signal peptide or transmembrane consensus sequences are reported in Table 3.

The results of HMMER analysis as reported in Tables 2 and 3 may support the results of BLAST analysis as reported in Table 1 or may suggest alternative or additional properties of template-encoded polypeptides not previously uncovered by BLAST or other analyses.

Template sequences are further analyzed using the bioinformatics tools listed in Table 7, or using sequence analysis software known in the art such as MACDNASIS PRO software (Hitachi Software Engineering, South San Francisco Calif.) and LASERGENE software (DNASTAR). Template sequences may be further queried against public databases such as the GenBank rodent, mammalian, vertebrate, prokaryote, and eukaryote databases.

The template sequences were translated to derive the corresponding longest open reading frame as presented by the polypeptide sequences. Alternatively, a polypeptide of the invention may begin at any of the methionine residues within the full length translated polypeptide. Polypeptide sequences were subsequently analyzed by querying against the GenBank protein database (GENPEPT, (GenBank version 121)). Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 6 shows sequences with homology to the polypeptides of the invention as identified by BLAST analysis against the GenBank protein (GENPEPT) database. Column 1 shows the polypeptide sequence identification number (SEQ ID NO:) for the polypeptide segments of the invention. Column 2 shows the reading frame used in the translation of the polynucleotide sequences encoding the polypeptide segments. Column 3 shows the length of the translated polypeptide segments. Columns 4 and 5 show the start and stop nucleotide positions of the polynucleotide sequences encoding the polypeptide segments. Column 6 shows the GenBank identification number (GI Number) of the nearest GenBank homolog. Column 7 shows the probability score for the match between each polypeptide and its GenBank homolog. Column 8 shows the annotation of the GenBank homolog.

V. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalize value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap:

VI. Tissue Distribution Profiling

A tissue distribution profile is determined for each template by compiling the cDNA library tissue classifications of its component cDNA sequences. Each. component sequence, is derived from a cDNA library constructed from a human tissue. Each human tissue is classified into one of the following categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. Template sequences, component sequences, and cDNA library/tissue information are found in the LIFESEQ GOLD database (Incyte Genomics, Palo Alto Calif.).

Table 5 shows the tissue distribution profile for the templates of the invention. For each template, the three most frequently observed tissue categories are shown in column 3, along with the percentage of component sequences belonging to each category. Only tissue categories with percentage values of ≧10% are shown. A tissue distribution of “widely distributed” in column 3 indicates percentage values of <10% in all tissue categories.

VII. Transcript Image Analysis

Transcript images are generated as described in Seilhamer et al., “Comparative Gene Transcript Analysis,” U.S. Pat. No. 5,840,484, incorporated herein by reference.

VIII. Extension of Polynucleotide Sequences and Isolation of a Full-length cDNA

Oligonucleotide primers designed using a dithp of the Sequence Listing are used to extend the nucleic acid sequence. One primer is synthesized to initiate 5′ extension of the template, and the other primer, to initiate 3′ extension of the template. The initial primers may be designed using OLIGO 4.06 software (National Biosciences, Inc. (National Biosciences), Plymouth Minn.), or another appropriate program, to be about 22 to 30 nucleotides in length, to have a GC content of about 50% or more, and to anneal to the target sequence at temperatures of about 68° C. to about 72° C.. Any stretch of nucleotides which would result in hairpin structures and primer-primer dimerzations are avoided. Selected human cDNA libraries are used to extend the sequence. If more than one extension is necessary or desired, additional or nested sets of primers are designed.

High fidelity amplification is obtained by PCR using methods well known in the art PCR is performed in 96-well plates using the PTC-200 thermal cycler (MJ Research). The reaction mix contains DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ are as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well is determined by dispensing 100 μl PICOGREEN quantitation reagent (0.25% (v/v); Molecular Probes) dissolved in 1× Tris-EDTA CE) and 0.5 μl of undiluted PCR product into each well of an opaque fluorimeter plate (Corning Incorporated (Corning), Corning N.Y.), allowing the DNA to bind to the reagent. The plate is scanned in a FLUOROSKAN II Labsystems Oy) to measure the fluorescence of the sample and to quantify the concentration of DNA A 5 μl to 10 μl aliquot of the reaction mixture is analyzed by electrophoresis on a 1% agarose mini-gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides are separated on low concentration (0.6 to 0.8%) agarose gels, fragments are excised, and agar digested with AGAR ACE (Promega). Extended clones are reilgated using T4 ligase (New England Biolabs, Inc., Beverly Mass.) into pUC 18 vector (Amersham Pharmacia Biotech), treated with Pfu DNA polymerase (Stratagene) to fill-in restriction site overhangs, and transfected into competent E. coli cells. Transformed cells are selected on antibiotic-containing media, individual colonies are picked and cultured overnight at 37° C. in 384-well plates in LB/2x carbenicillin liquid media.

The cells are lysed, and DNA is amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2min; Step 5: steps 2, 3, and 4 repeated 29 times; Step 6: 72° C., 5 min; Step 7: storage at 4° C. DNA is quantified by PICOGREEN reagent (Molecular Probes) as described above. Samples with low DNA recoveries are reamplified using the same conditions as described above. Samples are diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, the dithp is used to obtain regulatory sequences (promoters, introns, and enhancers) using the procedure above, oligonucleotides designed for such extension, and an appropriate genomic library.

IX. Labeling of Probes and Southern Hybridization Analyses

Hybridization probes derived from the dithp of the Sequence Listing are employed for screening cDNAs, mRNAs, or genomic DNA. The labeling of probe nucleotides between 100 and 1000 nucleotides in length is specifically described, but essentially the same procedure may be used with larger cDNA fragments. Probe sequences are labeled at room temperature for 30 minutes using a T4 polynucleotide kinase, γ³²P-ATP, and 0.5× One-Phor-All Plus (Amersham Pharmacia Biotech) buffer and purified using a ProbeQuant G-50 Microcolumn (Amersham Pharmacia Biotech). The probe mixture is diluted to 10⁷ dpm/μg/ml hybridization buffer and used in a typical membrane-based hybridization analysis.

The DNA is digested with a restriction endonuclease such as Eco RV and is electrophoresed through a 0.7% agarose gel. The DNA fragments are transferred from the agarose to nylon membrane (NYTRAN Plus, Schleicher & Schuell, Inc., Keene N. H.) using procedures specified by the manufacturer of the membrane. Prehybridization is carried out for three or more hours at 68° C., and hybridization is carried out overnight at 68° C. To remove non-specific signals, blots are sequentially washed at room temperature under increasingly stringent conditions, up to 0.1× saline sodium citrate (SSC) and 0.5% sodium dodecyl sulfate. After the blots are placed in a PHOSPHORIMAGER cassette (Molecular Dynamics) or are exposed to autoradiography film, hybridization patterns of standard and experimental lanes are compared. Essentially the same procedure is employed when screening RNA.

X. Chromosome Mapping of dithp

The cDNA sequences which were used to assemble SEQ ID NO:1-211 are compared with sequences from the Incyte LIFESEQ database and public domain databases using BLAST and other implementations of the Smith-Waterman algorithm. Sequences from these databases that match SEQ ID NO:1-211 are assembled into clusters of contiguous and overlapping sequences using assembly algorithms such as PHRAP (Table 7). Radiation hybrid and genetic mapping data available from public resources such as the Stanford Human Genome Center (SHGC), Whitehead Institute for Genome Research (WIGR), and Généthon are used to determine if any of the clustered sequences have been previously mapped. Inclusion of a mapped sequence in a cluster will result in the assignment of all sequences of that cluster, including its particular SEQ ID NO:, to that map location. The genetic map locations of SEQ ID NO:1-211 are described as ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters.

XI. Microarray Analysis

Probe Preparation from Tissue or Cell Samples

Total RNA is isolated from tissue samples using the guanidinium thiocyanate method and polyA⁺ RNA is purified using the oligo (dT) cellulose method Each polyA⁺ RNA sample is reverse transcribed using MMLV reverse-transcriptase, 0.05 pg/μl oligo-dT primer (21mer), 1×first strand buffer, 0.03 units/μl RNase inhibitor, 500 μM dATP, 500 μM dGTP, 500 μM d=TP, 40 μM dCTP, 40 μM dCTP-Cy3 (BDS) or dCTP-Cy5 (Amersham Pharmacia Biotech). The reverse transcription reaction is performed in a 25 ml volume containing 200 ng polyA⁺ RNA with GEMBRIGHT kits (Incyte). Specific control polyA⁺ RNAs are synthesized by in vitro transcription from non-coding yeast genomic DNA (W. Lei, unpublished). As quantitative controls, the control mRNAs at 0.002 ng, 0.02 ng, 0.2 ng, and 2 ng are diluted into reverse transcription reaction at ratios of 1:100,000, 1:10,000, 1:1000, 1:100 (w/w) to sample mRNA respectively. The control mRNAs are diluted into reverse transcription reaction at ratios of 1:3, 3:1, 1:10, 10:1, 1:25, 25:1 (w/w) to sample mRNA differential expression patterns. After incubation at 370 C for 2 br, each reaction sample (one with Cy3 and another with Cy5 labeling) is treated with 2.5 ml of 0.5M sodium hydroxide and incubated for 20 minutes at 85° C. to the stop the reaction and degrade the RNA Probes are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The probe is then dried to completion using a SpeedVAC (Savant Instruments Inc., HoIbrook N.Y.) and resuspended in 14 μl 5×SSC/0.2%

SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester, Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford, Mass.) for 30 minutes at 60° C. followed by washes in 0.2% SDS and distilled water as before.

Hybridization

Hybridization reactions contain 9 μl of probe mixture consisting of 0.2 μg each of Cy3 and Cy5 labeled cDNA synthesis products in 5×SSC, 0.2% SDS hybridization buffer. The probe mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 6321n for excitation of Cy5. The excitation laser light is focused on the array using a 20×microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the probe mix at a known concentration. A specific location on the array contains a complementary DNA sequence, allowing the intensity of the signal at that location to be correlated with a weight ratio of hybridizing species of 1:100,000. When two probes from different sources (e g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood, Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal win each element is then integrated to obtain a numerical value corresponding to the average intensity of the signal. The software used for signal analysis is the GEMTOOLS gene expression analysis program (Incyte).

XII. Complementary Nucleic Acids

Sequences complementary to the dithp are used to detect, decrease, or inhibit expression of the naturally occurring nucleotide. The use of oligonucleotides comprising from about 15 to 30 base pairs is typical in the art. However, smaller or larger sequence fragments can also be used. Appropriate oligonucleotides are designed from the dithp using OLIGO 4.06 software (National Biosciences) or other appropriate programs and are synthesized using methods standard in the art or ordered from a commercial supplier. To inhibit transcription, a complementary oligonucleotide is designed from the most unique 5′ sequence and used to prevent transcription factor binding to the promoter sequence. To inhibit translation, a complementary oligonucleotide is designed to prevent ribosomal binding and processing of the transcript

XIII. Expression of DITHP

Expression and purification of DITHP is accomplished using bacterial or virus-based expression systems. For expression of DITHP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express DITHP upon induction with isopropyl beta-D-thiogalactopyranoside (IPTG). Expression of DITHP in eukaryotic cells is achieved by infecting insect or mammalian cell lines with recombinant Autosraphica californica nuclear polyhedrosis virus (AcMNPV), commonly known as baculovirus. The nonessential polyhedrin gene of baculovirus is replaced with cDNA encoding DITHP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera fruiperda (Sf9) insect cells in most cases, or human hepatocytes, in some cases. Infection of the latter requires additional genetic modifications to baculovirus. (See e.g., Engelhard, supra; and Sandig, supra.)

In most expression systems, DITHP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from DITHP at specifically engineered sites. FLAG, an 8-amino acid peptide, enables immunoaffnity purification using commercially available monoclonal and polyclonal anti-FLAG antibodies (Eastman Kodak Company, Rochester N.Y.). 6-His, a stretch of six consecutive histidine residues, enables purification on metal chelate resins (QIAGEN). Methods for protein expression and purification are discussed in Ausubel (1995, supra, Chapters 10 and 16). Purified DITh=obtained by these methods can be used directly in the following activity assay.

XIV. Demonstration of DITHP Activity

DITHP activity is demonstrated through a variety of specific assays, some of which are outlined below.

Oxidoreductase activity of DITHP is measured by the increase in extinction coefficient of NAD(P)H coenzyme at 340 nm for the measurement of oxidation activity, or the decrease in extinction coefficient of NAD(P)H coenzyme at 340 nm for the measurement of reduction activity (Dalziel, K. (1963) J. Biol. Chem. 238:2850-2858). One of three substrates may be used: Asn-βGal, biocytidine, or ubiquinone-10. The respective subunits of the enzyme reaction, for example, cytochtome c₁-b oxidoreductase and cytochrome c, are reconstituted. The reaction mixture contains a)1-2 mg/ml DITHP; and b) 15 mM substrate, 2.4 mM NAD(P)⁺in 0.1 M phosphate buffer, pH 7.1 (oxidation reaction), or 2.0 mM NAD(P)H, in 0.1 M Na₂HPO₄ buffer, pH 7.4 (reduction reaction); in a total volume of 0.1 ml. Changes in absorbance at 340 nm (A340) are measured at 23.5° C. using a recording spectrophotometer (Shimadzu Scientific Instruments, Inc., Pleasanton Calif.). The amount of NAD(P)H is stoichiometrically equivalent to the amount of substrate initially present, and the change in A₃₄₀ is a direct measure of the amount of NAD(P)H produced; ΔA₃₄₀=6620[NADH]. Oxidoreductase activity of DITHP activity is proportional to the amount of NAD(P)H present in the assay.

Transferase activity of DITHP is measured through assays such as a methyl transferase assay in which the transfer of radiolabeled methyl groups between a donor substrate and an acceptor substrate is measured (Bokar, J. A. et al. (1994) J. Biol. Chem. 269:17697-17704). Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H]AdoMet (0.375 μM AdoMet) (DuPont-NEN), 0.6 μg DITHP, and acceptor substrate (0.4 μg [³⁵S]RNA or 6-mercaptopurine (6-MP) to 1 mM final concentration). Reaction mixtures are incubated at 30° C. for 30 minutes, then 65° C. for 5 minutes. The products are separated by chromatography or electrophoresis and the level of methyl transferase activity is determined by quantification of methyl-3H recovery.

DITHP hydrolase activity is measured by the hydrolysis of appropriate synthetic peptide substrates conjugated with various chromogenic molecules in which the degree of hydrolysis is quantified by spectrophotometric (or fluorometric) absorption of the released chromophore. (Beynon, R. J. and J. S. Bond (1994) Proteolytic Enzymes: A Practical Approach, Oxford University Press, New York N.Y., pp. 25-55) Peptide substrates are designed according to the category of protease activity as endopeptidase (serine, cysteine, aspartic proteases), animopeptidase (leucine aminopeptidase), or carboxypeptidase (Carboxypeptidase A and B, procollagen C-proteinase).

DITHP isomerase activity such as peptidyl prolyl cis/trans isomerase activity can be assayed by an enzyme assay described by Rahfeld, J. U., et al. (1994) (FEBS Let 352:180-184). The assay is performed at 10° C. in 35 mM HEPES buffer, pH 7.8, containing chymotrypsin (0.5 mg/ml) and DITHP at a variety of concentrations. Under these assay conditions, the substrate, Suc-Ala-Xaa-Pro-Phe-4-NA, is in equilibrium with respect to the prolyl bond, with 80-95% in trans and 5-20% in cis conformation. An aliquot (2 ul) of the substrate dissolved in dimethyl sulfoxide (10 mg/ml) is added to the reaction mixture described above. Only the cis isomer of the substrate is a substrate for cleavage by chymotrypsin. Thus, as the substrate is isomerized by DITHP, the product is cleaved by chymotrypsin to produce 4-nitroanilide, which is detected by it's absorbance at 390 nm. 4 Nitroanilide appears in a time-dependent and a DITHP concentration-dependent manner.

An assay for DITHP activity associated with growth and development measures cell proliferation as the amount of newly initiated DNA synthesis in Swiss mouse 3T3 cells. A plasmid containing polynucleotides encoding DITHP is transfected into quiescent 3T3 cultured cells using methods well known in the art. The transiently transfected cells are then incubated in the presence of [³H]thymidine, a radioactive DNA precursor. Where applicable, varying amounts of DITHP ligand are added to the transfected cells. Incorporation of [³H]thymidine into acid-precipitable DNA is measured over an appropriate time interval, and the amount incorporated is directly proportional to the amount of newly synthesized DNA.

Growth factor activity of DITHP is measured by the stimulation of DNA synthesis in Swiss mouse 3T3 cells (McKay, I. and L. Leigh, eds. (1993) Growth Factors: A Practical Approach, Oxford University Press, New York N.Y.). Initiation of DNA synthesis indicates the cells' entry into the mitotic cycle and their commitment to undergo later division 3T3 cells are competent to respond to most growth factors, not only those that are mitogenic, but also those that are involved in embryonic induction. This competence is possible because the in vivo specificity demonstrated by some growth factors is not necessarily inherent but is determined by the responding tissue. In this assay, varying amounts of DITHP are added to quiescent 3T3 cultured cells in the presence of [³H]thymidine, a radioactive DNA precursor. DITHP for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA A linear dose-response curve over at least a hundred-fold DITHP concentration range is indicative of growth factor activity. One unit of activity per milliliter is defined as the concentration of DITHP producing a 50% response level, where 100% represents maximal incorporation of [³H]thymidine into acid-precipitable DNA.

Alternatively, an assay for cytokine activity of DITHP measures the proliferation of leukocytes. In this assay, the amount of tritiated thymidine incorporated into newly synthesized DNA is used to estimate proliferative activity. Varying amounts of DITHP are added to cultured leukocytes, such as granulocytes, monocytes, or lymphocytes, in the presence of [³H]thymidine, a radioactive DNA precursor. DITHP for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold DITHP concentration range is indicative of DITHP activity. One unit of activity per milliliter is conventionally defined as the concentration of DITHP producing a 50% response level, where 100% represents maximal incorporation of [³H]thymidine into acid-precipitable DNA.

An alternative assay for DITHP cytokine activity utilizes a Boyden micro chamber (Neuroprobe, Cabin John M.D.) to measure leukocyte chemotaxis (Vicari, supra). In this assay, about 10⁵ migratory cells such as macrophages or monocytes are placed in cell culture media in the upper compartment of the chamber. Varying dilutions of DITHP are placed in the lower compartment. The two compartments are separated by a 5 or 8 micron pore polycarbonate filter (Nucleopore, Pleasanton Calif.). After incubation at 37° C. for 80 to 120 minutes, the filters are fixed in methanol and stained with appropriate labeling agents. Cells which migrate to the other side of the filter are counted using standard microscopy. The chemotactic index is calculated by dividing the number of migratory cells counted when DITHP is present in the lower compartment by the number of migratory cells counted when only media is present in the lower compartment. The chemotactic index is proportional to the activity of DITHP.

Alternatively, cell lines or tissues transformed with a vector containing dithp can be assayed for DITHP activity by immunoblotting. Cells are denatured in SDS in the presence of β-mercaptoethanol, nucleic acids removed by ethanol precipitation, and proteins purified by acetone precipitation. Pellets are resuspended in 20 mM tris buffer at pH 7.5 and incubated with Protein G-Sepharose pre-coated with an antibody specific for DITHP. After washing, the Sepharose beads are boiled in electrophoresis sample buffer, and the eluted proteins subjected to SDS-PAGE. The SDS-PAGE is transferred to a nitrocellulose membrane for immunoblotting, and the DITHP activity is assessed by visualizing and quantifying bands on the blot using the antibody specific for DITHP as the primary antibody and ¹²⁵I-labeled IgG specific for the primary antibody as the secondary antibody.

DITHP kinase activity is measured by phosphorylation of a protein substrate using γ-labeled [³²p]-ATP and quantitation of the incorporated radioactivity using a radioisotope counter. DITHP is incubated with the protein substrate, [³²P]-ATP, and an appropriate kinase buffer. The [³²P] incorporated into the product is separated from free [³²]-ATP by electrophoresis and the incorporated [³²P] is counted. The amount of [³²P] recovered is proportional to the kinase activity of DITHP in the assay. A determination of the specific amino acid residue phosphorylated is made by phosphoamino acid analysis of the hydrolyzed protein.

In the alternative, DITHP activity is measured by the increase in cell proliferation resulting from transformation of a mammalian cell line such as COS7, HeLa or CHO with an eukaryotic expression vector encoding DITHP. Eukaryotic expression vectors are commercially available, and the techniques to introduce them into cells are well known to those skilled in the art. The cells are incubated for 48-72 hours after transformation under conditions appropriate for the cell line to allow expression of DITHP. Phase microscopy is then used to compare the mitotic index of transformed versus control cells. An increase in the mitotic index indicates DITHP activity.

In a further alternative, an assay for DITHP signaling activity is based upon the ability of GPCR family proteins to modulate G protein-activated second messenger signal transduction pathways (e.g., cAMP; Gaudin, P. et al. (1998) J. Biol. Chem. 273:4990-4996). A plasmid encoding full length DITHP is transfected into a mammalian cell line (e.g., Chinese hamster ovary (CHO) or human embryonic kidney (HEK-293) cell lines) using methods well-known in the art. Transfected cells are grown in 12-well trays in culture medium for 48 hours, then the culture medium is discarded, and the attached cells are gently washed with PBS. The cells are then incubated in culture medium with or without ligand for 30 minutes, then the medium is removed and cells lysed by treatment with 1 M perchloric acid. The cAMP levels in the lysate are measured by radioimmunoassay using methods well-known in the art. Changes in the levels of cAMP in the lysate from cells exposed to ligand compared to those without ligand are proportional to the amount of DITHP present in the transfected cells.

Alternatively, an assay for DITHP protein phosphatase activity measures the hydrolysis of P-nitrophenyl phosphate (PNPP). DITHP is incubated together with PNPP in HEPES buffer pH 7.5, in the presence of 0.1% β-mercaptoethanol at 37° C. for 60 min. The reaction is stopped by the addition of 6 ml of 10 N NaOH, and the increase in light absorbance of the reaction mixture at 410 nm resulting from the hydrolysis of PNPP is measured using a spectrophotometer. The increase in light absorbance is proportional to the phosphatase activity of DITHP in the assay (Diamond, R. H. et al (1994) Mol Cell Biol 14:3752-3762).

An alternative assay measures DITHP-mediated G-protein signaling activity by monitoring the mobilization of Ca⁺⁺ as an indicator of the signal transduction pathway stimulation. (See, e.g., Grynkievicz, G. et al. (1985) J. Biol. Chem. 260:3440; McColl, S. et al. (1993) J. Immunol. 150:4550-4555; and Aussel, C. et al. (1988) J. Immunol. 140:215-220). The assay requires preloading neutrophils or T cells with a fluorescent dye such as FURA-2 or BCECF (Universal Imaging Corp, Westchester Pa.) whose emission characteristics are altered by Ca⁺⁺ binding. When the cells are exposed to one or more activating stimuli artificially (e.g., anti-CD3 antibody ligation of the T cell receptor) or physiologically (e.g., by allogeneic stimulation), Ca⁺⁺ flux takes place. This flux can be observed and quantified by assaying the cells in a fluorometer or fluorescent activated cell sorter. Measurements of Ca⁺⁺ flux are compared between cells in their normal state and those transfected with DITHP. Increased Ca⁺⁺ mobilization attributable to increased DITHP concentration is proportional to DITHP activity.

DITHP transport activity is assayed by measuring uptake of labeled substrates into Xenopus laevis oocytes. Oocytes at stages V and VI are injected with DITHP mRNA (10 ng per oocyte) and is incubated for 3 days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH, 50 μg/ml gentamycin, pH 7.8) to allow expression of DITHP protein. Oocytes are then transferred to standard uptake medium (100 mM NaCl, 2 mM KCl, 1 mM CaCl₂, 1 mM MgCl₂, 10 mM Hepes/Tris pH 7.5). Uptake of various substrates (e.g., amino acids, sugars, drugs, ions, and neurotransmitters) is initiated by adding labeled substrate (e.g. radiolabeled with ³H, fluorescently labeled with rhodamine, etc.) to the oocytes. After incubating for 30 minutes, uptake is terminated by washing the oocytes three times in Na⁺-free medium, measuring the incorporated label, and comparing with controls. DITHP transport activity is proportional to the level of internalized labeled substrate.

DITHP transferase activity is demonstrated by a test for galactosyltransferase activity. This can be determined by measuring the transfer of radiolabeled galactose from UDP-galactose to a GlcNAc-terminated oligosaccharide chain (Kolbinger, F. et al. (1998) J. Biol. Cheri 273:58-65). The sample is incubated with 14 μl of assay stock solution (180 mM sodium cacodylate, pH 6.5, 1 mg/ml bovine serum albumin, 0.26 mM UDP-galactose, 2 μl of UDP-[³H]galactose), 1 μl of MnCl₂ (500 mM), and 2.5 μl of GlcNAcβO—(CH₂)₈—CO₂Me (37 mg/ml in dimethyl sulfoxide) for 60 minutes at 37° C. The reaction is quenched by the addition of 1 ml of water and loaded on a C18 Sep-Pak cartridge (Waters), and the column is washed twice with 5 ml of water to remove unreacted UDP-[³H]galactose. The [³H]galactosylated GlcNAcβO—(CH₂)₈—CO₂Me remains bound to the column during the water washes and is eluted with 5 ml of methanol. Radioactivity in the eluted material is measured by liquid scintillation counting and is proportional to galactosyltransferase activity in the starting sample.

In the alternative, DITHP induction by heat or toxins may be demonstrated using primary cultures of human fibroblasts or human cell lines such as CCL-13, HEK293, or HEP G2 (ATCC). To heat induce DITHP expression, aliquots of cells are incubated at 42° C. for 15, 30, or 60 minutes. Control aliquots are incubated at 37° C. for the same time periods. To induce DITHP expression by toxins, aliquots of cells are treated with 100 μM arsenite or 20 mM azetidine-2-carboxylic acid for 0, 3, 6, or 12 hours. After exposure to heat, arsenite, or the amino acid analogue, samples of the treated cells are harvested and cell lysates prepared for analysis by western blot. Cells are lysed in lysis buffer containing 1% Nonidet P40, 0.15 M NaCl, 50 mM Tris-HCl, 5 mM EDTA, 2 mM N-ethylmaleimide, 2 mM phenylmethylsulfonyl fluoride, 1 mg/ml leupeptin, and 1 mg/ml pepstatin. Twenty micrograms of the cell lysate is separated on an 8% SDS-PAGE gel and transferred to a membrane. After blocking with 5% nonfat dry milk/phosphate-buffered saline for 1 h, the membrane is incubated overnight at 4° C. or at room temperature for 24 hours with a 1:1000 dilution of anti-DITHP serum in 2% nonfat dry milk/phosphate-buffered saline. The membrane is then washed and incubated with a 1:1000 dilution of horseradish peroxidase-conjugated goat anti-rabbit IgG in 2% dry milk/phosphate-buffered saline. After washing with 0.1% Tween 20 in phosphate-buffered saline, the DITHP protein is detected and compared to controls using chemiluminescence.

Alternatively, DITHP protease activity is measured by the hydrolysis of appropriate synthetic peptide substrates conjugated with various chromogenic molecules in which the degree of hydrolysis is quantified by spectrophotometric (or fluorometric) absorption of the released chromophore (Beynon, R. J. and J. S. Bond (1994) Proteolytic Enzymes: A Practical Approach, Oxford University Press, New York, N.Y., pp.25-55). Peptide substrates are designed according to the category of protease activity as endopeptidase (serine, cysteine, aspartic proteases, or metalloproteases), aminopeptidase (leucine aminopeptidase), or carboxypeptidase (carboxypeptidases A and B, procollagen C-proteinase). Commonly used chromogens are 2-naphthylamine, 4-nitroaniline, and furylacrylic acid. Assays are performed at ambient temperature and contain an aliquot of the enzyme and the appropriate substrate in a suitable buffer. Reactions are carried out in an optical cuvette, and the increase/decrease in absorbance of the chromogen released during hydrolysis of the peptide substrate is measured. the change in absorbance is proportional to the DITHP protease activity in the assay.

In the alternative, an assay for DITHP protease activity takes advantage of fluorescence resonance energy transfer (FRET) that occurs when one donor and one acceptor fluorophore with an appropriate spectral overlap are in close proximity. A flexible peptide linker containing a cleavage site specific for PRTS is fused between a red-shifted variant (RSGFP4) and a blue variant (BFP5) of Green Fluorescent Protein. This fusion protein has spectral properties that suggest energy transfer is occurring from BFP5 to RSGFP4. When the fusion protein is incubated with DITHP, the substrate is cleaved, and the two fluorescent proteins dissociate. This is accompanied by a marked decrease in energy transfer which is quantified by comparing the emission spectra before and after the addition of DITHP (Mitra, R. D. et al (1996) Gene 173:13-17). This assay can also be performed in living cells. In this case the fluorescent substrate protein is expressed constitutively in cells and DITHP is introduced on an inducible vector so that FRET can be monitored in the presence and absence of DITHP (Sagot, I. et al (1999) FEBS Lett. 447:53-57).

A method to determine the nucleic acid binding activity of DITHP involves a polyacrylamide gel mobility-shift assay. In preparation for this assay, DITHP is expressed by transforming a mammalian cell line such as COS7, HeLa or CHO with a eukaryotic expression vector containing DITHP cDNA. The cells are incubated for 48-72 hours after transformation under conditions appropriate for the cell line to allow expression and accumulation of DITHP. Extracts containing solubilized proteins can be prepared from cells expressing DITHP by methods well known in the art Portions of the extract containing DITHP are added to [³²P]-labeled RNA or DNA. Radioactive nucleic acid can be synthesized in vitro by techniques well known in the art The mixtures are, incubated at 25° C. in the presence of RNase- and DNase-inhibitors under buffered conditions for 5-10 minutes. After incubation, the samples are analyzed by polyacrylamide gel electrophoresis followed by autoradiography. The presence of a band on the autoradiogram indicates the formation of a complex between DITHP and the radioactive transcript. A band of similar mobility will not be present in samples prepared using control extracts prepared from untransformed cells.

In the alternative, a method to determine the methylase activity of a DITHP measures transfer of radiolabeled methyl groups between a donor substrate and an acceptor substrate. Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H]AdoMet (0.375 μM AdoMet) (DuPont-NEN), 0.6 μg DITHP, and acceptor substrate (e.g., 0.4 μg [³⁵S]RNA, or 6-mercaptopurine (6-MP) to 1 mM final concentration). Reaction mixtures are incubated at 30° C. for 30 minutes, then 65° C. for 5 minutes. Analysis of [methyl-³H]RNA is as follows: 1) 50 μl of 2×loading buffer (20 mM Tris-HCl, pH 7.6, 1 M LiCl, 1 mM EDTA, 1% sodium dodecyl sulphate (SDS)) and 50 μl oligo d(T)-cellulose (10 mg/ml in 1×loading buffer) are added to the reaction mixture, and incubated at ambient temperature with shaking for 30 minutes. 2) Reaction mixtures are transferred to a 96-well filtration plate attached to a vacuum apparatus. 3) Each sample is washed sequentially with three 2.4 ml aliquots of 1×oligo d(T) loading buffer containing 0.5% SDS, 0.1% SDS, or no SDS. and 4) RNA is eluted with 300 μl of water into a 96-well collection plate, transferred to scintillation vials containing liquid scintillant, and radioactivity determined. Analysis of [methyl-³H]6-MP is as follows: 1) 500 μl 0.5 M borate buffer, pH 10.0, and then 2.5 ml of 20% (v/v) isoamyl alcohol in toluene are added to the reaction mixtures. 2) The samples mixed by vigorous vortexing for ten seconds. 3) After centrifugation at 700 g for 10 minutes, 1.5 ml of the organic phase is transferred to scintillation vials containing 0.5 ml absolute ethanol and liquid scintillant, and radioactivity determined. and 4) Results are corrected for the extraction of 6-MP into the organic phase (approximately 41%).

An assay for adhesion activity of DITHP measures the disruption of cytoskeletal filament networks upon overexpression of DITHP in cultured cell lines (Rezniczek, G. A. et al. (1998) J. Cell Biol. 141:209-225). cDNA encoding DITHP is subcloned into a mammalian expression vector that drives high levels of cDNA expression. This construct is transfected into cultured cells, such as rat kangaroo PtK2 or rat bladder carcinoma 804G cells. Actin filaments and intermediate filaments such as keratin and vimentin are visualized by immunofluorescence microscopy using antibodies and techniques well known in the art. The configuration and abundance of cytoskeletal filaments can be assessed and quantified using confocal imaging techniques. In particular, the bundling and collapse is of cytoskeletal filament networks is indicative of DITHP adhesion activity.

Alternatively, an assay for DITHP activity measures the expression of DITHP on the cell surface. cDNA encoding DITHP is transfected into a non-leukocytic cell line. Cell surface proteins are labeled with biotin (de la Fuente, M. A. et al. (1997) Blood 90:2398-2405). Immunoprecipitations are performed using DITHP-specific antibodies, and immunoprecipitated samples are analyzed using SDS-PAGE and immunoblotting techniques. The ratio of labeled immunoprecipitant to unlabeled immunoprecipitant is proportional to the amount of DITHP expressed on the cell surface.

Alternatively, an assay for DITHP activity measures the amount of cell aggregation induced by overexpression of DITHP. In this assay, cultured cells such as NIH3T3 are transfected with cDNA encoding DITHP contained within a suitable mammalian expression vector under control of a strong promoter. Cotransfection with cDNA encoding a fluorescent marker protein, such as Green Fluorescent Protein (CLONTECH), is useful for identifying stable transfectants. The amount of cell agglutination, or clumping, associated with transfected cells is compared with that associated with untransfected cells. The amount of cell agglutination is a direct measure of DITHP activity.

DITHP may recognize and precipitate antigen from serum This activity can be measured by the quantitative precipitin reaction (Golub, E. S. et al. (1987) Immunology: A Synthesis, Sinauer Associates, Sunderland M A, pages 113-115). DITHP is isotopically labeled using methods known in the art. Various serum concentrations are added to constant amounts of labeled DITHP. DITHP-antigen complexes precipitate out of solution and are collected by centrifugation. The amount of precipitable DITHP-antigen complex is proportional to the amount of radioisotope detected in the precipitate. The amount of precipitable DITHP-antigen complex is plotted against the serum concentration. For various serum concentrations, a characteristic precipitation curve is obtained, in which the amount of precipitable DITHP-antigen complex initially increases proportionately with increasing serum concentration, peaks at the equivalence point, and then decreases proportionately with further increases in serum concentration. Thus, the amount of precipitable DITHP-antigen complex is a measure of DITHP activity which is characterized by sensitivity to both limiting and excess quantities of antigen.

A microtubule motility assay for DITHP measures motor protein activity. In this assay, recombinant DITHP is immobilized onto a glass slide or similar substrate. Taxol-stabilized bovine brain microtubules (commercially available) in a solution containing ATP and cytosolic extract are perfused onto the slide. Movement of microtubules as driven by DITHP motor activity can be visualized and quantified using video-enhanced light microscopy and image analysis techniques. DITHP motor protein activity is directly proportional to the frequency and velocity of microtubule movement.

Alternatively, an assay for DITHP measures the formation of protein filaments in vitro. A solution of DITHP at a concentration greater than the “critical concentration” for polymer assembly is applied to carbon-coated grids. Appropriate nucleation sites may be supplied in the solution. The grids are negative stained with 0.7% (w/v) aqueous uranyl acetate and examined by electron microscopy. The appearance of filaments of approximately 25 nm (microtubules), 8 nm (actin), or 10 nm (intermediate filaments) is a demonstration of protein activity.

DITHP electron transfer activity is demonstrated by oxidation or reduction of NADP. Substrates such as Asn-βGal, biocytidine, or ubiquinone-10 may be used. The reaction mixture contains 1-2 mg/ml HORP, 15 mM substrate, and 2.4 mM NAD(P)⁺in 0.1 M phosphate buffer, pH 7.1 (oxidation reaction), or 2.0 mM NAD(P)H, in 0.1 M Na₂HPO₄ buffer, pH 7.4 (reduction reaction); in a total volume of 0.1 ml. FAD may be included with NAD, according to methods well known in the art. Changes in absorbance are measured using a recording spectrophotometer. The amount of NAD(P)H is stoichiometrically equivalent to the amount of substrate initially present, and the change in A₃₄₀ is a direct measure of the amount of NAD(P)H produced; ΔA³⁴⁰⁼⁶⁶²⁰[NADH]. DITHP activity is proportional to the amount of NAD(P)H present in the assay. The increase in extinction coefficient of NAD(P)H coenzyme at 340 nm is a measure of oxidation activity, or the decrease in extinction coefficient of NAD(P)H coenzyme at 340 nm is a measure of reduction activity (Dalziel, K (1963) J. Biol. Chen 238:2850-2858).

DITHP transcription factor activity is measured by its ability to stimulate transcription of a reporter gene (Liu, H. Y. et al. (1997) EMBO J. 16:5289-5298). The assay entails the use of a well characterized reporter gene construct, LexAₒₚ-LacZ, that consists of LexA DNA transcriptional control elements (LexAₒₚ) fused to sequences encoding the E. coli Lac enzyme. The methods for constructing and expressing fusion genes, introducing them into cells, and measuring LacZ enzyme activity, are well known to those skilled in the art. Sequences encoding DITHP are cloned into a plasmid that directs the synthesis of a fusion protein, LexA-DITHP, consisting of DITHP and a DNA binding domain derived from the LexA transcription factor. The resulting plasmid, encoding a LexA-DITHP fusion protein, is introduced into yeast cells along with a plasmid containing the LexAₒₚ-LacZ reporter gene. The amount of LacZ enzyme activity associated with LexA-DITHP transfected cells, relative to control cells, is proportional to the amount of transcription stimulated by the DITHP.

Chromatin activity of DITHP is demonstrated by measuring sensitivity to DNase I (Dawson, B. A. et al. (1989) J. Biol. Chem. 264:12830-12837). Samples are treated with DNase 1, followed by insertion of a cleavable biotinylated nucleotide analog, 5-[(N-biotinamido)hexanoamido-ethyl-1,3-thiopropionyl-3-aminoallyl]-2′-deoxyuridine 5′-triphosphate using nick-repair techniques well known to those skilled in the art Following purification and digestion with EcoRI restriction endonuclease, biotinylated sequences are affinity isolated by sequential binding to streptavidin and biotincellulose.

Another specific assay demonstrates the ion conductance capacity of DITHP using an electrophysiological assay. DITHP is expressed by transforming a mammalian cell line such as COS7. HeLa or CHO with a eukaryotic expression vector encoding DITH. Eukaryotic expression vectors are commercially available, and the techniques to introduce them into cells are well known to those skilled in the art. A small amount of a second plasmid, which expresses any one of a number of marker genes such as β-galactosidase, is co-transformed into the cells in order to allow rapid identification of those cells which have taken up and expressed the foreign DNA The cells are incubated for 48-72 hours after transformation under conditions appropriate for the cell line to allow expression and accumulation of DITHP and β-galactosidase. Transformed cells expressing β-galactosidase are stained blue when a suitable colorimetric substrate is added to the culture media under conditions that are well known in the art. Stained cells are tested for differences in membrane conductance due to various ions by electrophysiological techniques that are well known in the art. Untransformed cells, and/or cells transformed with either vector sequences alone or β-galactosidase sequences alone, are used as controls and tested in parallel. The contribution of DITHP to cation or anion conductance can be shown by incubating the cells using antibodies specific for either DITHP. The respective antibodies will bind to the extracellular side of DITHP, thereby blocking the pore in the ion channel, and the associated conductance.

XV. Functional Assays

DITHP function is assessed by expressing dithp at physiologically elevated levels in mammalian cell culture systems. cDNA is subcloned into a mammalian expression vector containing a strong promoter that drives high levels of cDNA expression Vectors of choice include pCMV SPORT (Life Technologies) and pCR3.1 (Invitrogen Corporation, Carlsbad Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected.

Expression of a marker protein provides a means to distinguish transfected cells from nontransfected cells and is a reliable predictor of cDNA expression from the recombinant vector. Marker proteins of choice include, e.g., Green Fluorescent Protein (GFP; CLONTECH), CD64, or a CD64GFP fusion protein. Flow cytometry (FCM), an automated laser optics-based technique, is used to identity transfected cells expressing GFP or CD64-GFP and to evaluate the apoptotic state of the cells and other cellular properties.

FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane, composition as measured by the binding of fluorescein-conjugated Annexin V protein to the cell surface. Methods in flow cytometry are discussed in Ormerod, M. G. (1994) Flow Cytometry, Oxford, New York N.Y.

The influence of DITHP on gene expression can be assessed using highly purified populations of cells transfected with sequences encoding DITHP and either CD64 or CD64GFP. CD64 and CD64GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Inc., Lake Success N.Y.). mRNA can be purified from the cells using methods well known by those of skill in the art Expression of mRNA encoding DITHP and other genes of interest can be analyzed by northern analysis or microarray techniques.

XVI. Production of Antibodies

DITHP substantially purified using polyacrylamide gel electophoresis (PAGE; see, e.g., Harrington, M. G. (1990) Methods Enzymol. 182:488-495), or other purification techniques, is used to immunize rabbits and to produce antibodies using standard protocols.

Alternatively, the DITHP amino acid sequence is analyzed using LASERGENE software (DNASTAR) to determine regions of high immunogenicity, and a corresponding peptide is synthesized and used to raise antibodies by means known to those of skill in the art Methods for selection of appropriate epitopes, such as those near the C-terminus or in hydrophilic regions are well described in the art. (See, e.g., Ausubel, 1995, supra, Chapter 11.) Typically, peptides 15 residues in length are synthesized using an ABI 431A peptide synthesizer (Applied Biosystems) using fmoc-chemistry and coupled to KLH (Sigma) by reaction with N-maleimidobenzoyl-N-hydroxysuccinimide ester (MBS) to increase immunogenicity. (See, e.g., Ausubel, supra.) Rabbits are immunized with the peptide-KLH complex in complete Freund's adjuvant Resulting antisera are tested for antipeptide activity by, for example, binding the peptide to plastic, blocking with 1% BSA, reacting with rabbit antisera, washing, and reacting with radio-iodinated goat anti-rabbit IgG. Antisera with antipeptide activity are tested for anti-DITHP activity using protocols well known in the art, including ELISA, RIA, and immunoblotting.

XVII. Purification of Naturally Occurring DITHP Using Specific Antibodies

Naturally occurring or recombinant DITHP is substantially purified by immunoaffinity chromatography using antibodies specific for DITHP. An immunoaffinity column is constructed by covalently coupling anti-DITHP antibody to an activated chromatographic resin, such as CNBr-activated SEPHAROSE (Amersham Pharmacia Biotech). After the coupling, the resin is blocked and washed according to the manufacturer's instructions.

Media containing DITHP are passed over the immunoaffinity column, and the column is washed under conditions that allow the preferential absorbance of DITHP (e.g., high ionic strength buffers in the presence of detergent). The column is eluted under conditions that disrupt antibody/DITHP binding (e.g., a buffer of pH 2 to pH 3, or a high concentration of a chaotrope, such as urea or thiocyanate ion), and DITHP is collected.

XVIII. Identification of Molecules Which Interact with DITHP

DITHP, or biologically active fragments thereof, are labeled with ¹²⁵I Bolton-Hunter reagent (See, e.g., Bolton, A. E. and W. M. Hunter (1973) Biochem. J. 133:529-539.) Candidate molecules previously arrayed in the wells of a multi-well plate are incubated with the labeled DITHP, washed, and any wells with labeled DITHP complex are assayed Data obtained using different concentrations of DITHP are used to calculate values for the number, affinity, and association of DITHP with the candidate molecules.

Alternatively, molecules interacting with DITHP are analyzed using the yeast two-hybrid system as described in Fields, S. and O. Song (1989) Nature 340:245-246, or using commercially available kits based on the two-hybrid system, such as the MATCHMAKER system (CLONTECH).

DITHP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K et al. (2000) U.S. Pat. No. 6,057,101).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1

| SEQ | | GI | | |
|---|---|---|---|---|
| ID NO: | Template ID | Number | Probability Score | Annotation |
| 1 | LG:1040582.1:2000FEB18 | g178480 | 1.00E−92 | Human aldehyde reductase mRNA, complete cds. |
| 2 | LG:453570.1:2000FEB18 | g2909424 | 2.40E−65 | Glyoxalase I |
| 3 | LG:408751.3:2000FEB18 | g3608122 | 4.40E−74 | dihydropyrimidinase |
| 4 | LI:090574.1:2000FEB01 | g29600 | 5.00E−85 | carbonic anhydrase I (AA 1-261) |
| 5 | LI:229932.2:2000FEB01 | g1835116 | 2.00E−63 | acetyl-CoA synthetase |
| 6 | LI:332176.1:2000FEB01 | g2104689 | 0 | alpha glucosidase II, alpha subunit |
| 7 | LI:403248.2:2000FEB01 | g63713 | 4.00E−23 | ornithine decarboxylase |
| 8 | LG:220992.1:2000MAY19 | g10435462 | 0 | unnamed protein product (Homo sapiens) |
| 9 | LG:1094571.1:2000MAY19 | g7023634 | 4.00E−92 | unnamed protein product (Homo sapiens) |
| 10 | LI:350754.4:2000MAY01 | g307504 | 0 | transglutaminase E3 (Homo sapiens) |
| 11 | LI:255828.29:2000MAY01 | g189998 | 9.00E−65 | M2-type pyruvate kinase (Homo sapiens) |
| 12 | LI:1190263.1:2000MAY01 | g2576305 | 1.00E−172 | arylsulphatase (Homo sapiens) |
| 13 | LG:270916.2:2000FEB18 | g2088668 | 1.20E−11 | similar to Achlya amblsexualis antheridiol steroid receptor (NID:g166306) |
| 14 | LG:999414.3:2000FEB18 | g3861482 | 0 | Human chromosome 3, olfactory receptor pseudogene cluster 1, |
| | | | | complete sequence, and myosin light chain kinase (MLCK) |
| | | | | pseudogene, partial sequence. |
| 15 | LG:429446.1:2000FEB18 | g2358042 | 0 | Human T-cell receptor alpha delta locus from bases 501613 to 752736 |
| | | | | (section 3 of 5) of the Complete Nucleotide Sequence. |
| 16 | LI:057229.1:2000FEB01 | g10439739 | 2.00E−19 | unnamed protein product (Homo sapiens) |
| 17 | LI:351965.1:2000FEB01 | g2358042 | 0 | Human T-cell receptor alpha delta locus from bases 501613 to 752736 |
| | | | | (section 3 of 5) of the Complete Nucleotlde Sequence. |
| 18 | LG:068682.1:2000FEB18 | g404634 | 1.10E−31 | serine/threonine kinase |
| 19 | LG:242665.1:2000FEB18 | g2117166 | 1.00E−160 | Ras like GTPase (Homo sapiens) |
| 20 | LG:241743.1:2000FEB18 | g5763838 | 9.70E−49 | dJ593C16.1 (ras GTPase activating protein) |
| 21 | LI:034212.1:2000FEB01 | g1469876 | 0 | The KIAA0147 gene product is related to adenylyl cyclase. |
| 22 | LG:344886.1:2000MAY19 | g7008402 | 2.00E−89 | kappa B-ras 1 (Homo sapiens) |
| 23 | LG:228930.1:2000MAY19 | g206218 | 4.50E−87 | phospholipase C-1 |
| 24 | LG:338927.1:2000MAY19 | g3599940 | 3.00E−57 | faclogenital dysplasia protein 2 (Mus musculus) |
| 25 | LG:898771.1:2000MAY19 | g508528 | 5.00E−58 | myocyte nuclear factor (Mus musculus) |
| 26 | LI:257664.67:2000MAY01 | g183399 | 1.00E−142 | Human guanine nucleotide-binding protein alpha-subunit gene (G-s- |
| | | | | alpha), exon 3. |
| 27 | LI:001496.2:2000MAY01 | g3005085 | 1.00E−177 | hook1 protein (Homo sapiens) |
| 28 | LI:1085273.2:2000MAY01 | g1781037 | 0 | neuronal tyrosine threonine phosphatase 1 (Mus musculus) |
| 29 | LI:333138.2:2000MAY01 | g2077934 | 1.00E−164 | Protein Kinase (Rattus norvegicus) |
| 30 | LI:338927.1:2000MAY01 | g3599940 | 6.00E−45 | faciogenital dysplasia protein 2 (Mus musculus) |
| 31 | LG:335558.1:2000FEB18 | g1181619 | 5.00E−97 | a variant of TSC-22 (Gallus gallus) |
| 32 | LG:998283.7:2000FEB18 | g6683492 | 1.00E−105 | bromodomain PHD finger transcription factor (Homo sapiens) |
| 33 | LI:402739.1:2000FEB01 | g4164151 | 6.00E−34 | AhR repressor |
| 34 | LI:175223.1:2000FEB01 | g2745892 | 2.00E−11 | Y box transcription factor |
| | | | | supported by Genscan and several ESTs: C83049 (NID:g3062006), |
| | | | | AA823760 (NID:g2893628), AA215791 (NID:g1815572), AI095488 |
| 35 | LG:981076.2:2000MAY19 | g3924670 | 1.00E−59 | (NID:a3434464), and AA969095 (NID:a3144275) (Homo sapiens) |
| 36 | LI:1008973.1:2000MAY01 | g6939732 | 2.00E−52 | transcription factor Elongin A2 (Homo sapiens) |
| 37 | LI:1190250.1:2000MAY01 | g3757892 | 4.00E−66 | enhancer of polycomb (Mus musculus) |
| 38 | LG:021371.3:2000FEB18 | g984814 | 1.40E−60 | zinc finger protein |
| 39 | LG:475404.1:2000FEB18 | g487784 | 5.00E−36 | Human zinc finger protein ZNF136. |
| 40 | LG:979406.2:2000FEB18 | g4325310 | 3.60E−1 1 | zinc-finger protein 7 |
| 41 | LG:410726.1:2000FEB1B | g6002480 | 2.60E−39 | BWSCR2 associated zinc-finger protein BAZ2 |
| 42 | LG:200005.1:2000FEB18 | g1504006 | 3.20E−25 | similarto human ZFY protein. |
| 43 | LG:1076828.1:2000FEB18 | g498720 | 2.00E−33 | Human HZF10 mRNA for zinc finger protein. |
| 44 | LG:1076931.1:2000FEB18 | g498151 | 2.00E−52 | Human mRNA for KIAA0065 gene, partial cds. |
| 45 | LG:1078121.1:2000FEB18 | g186773 | 2.00E−47 | Human Kruppel related zinc finger protein (HTF10) mRNA, complete cds. |
| 46 | LG:1079203.1:2000FEB18 | g1017721 | 0 | Human repressor transcriptional factor (ZNF85) mRNA, complete cds. |
| 47 | LG:1082586.1:2000FEB18 | g7959207 | 1.00E−19 | KIAA1473 protein (Homo sapiens) |
| 48 | LG:1082774.1:2000FEB18 | g184451 | 0 | Human Krueppel-related DNA-binding protein (TF9 PF4) mRNA, 5′ cds. |
| 49 | LG:1082775.1:2000FEB18 | g506502 | 3.50E−36 | NK10 |
| 50 | LG:1083120.1:2000FEB18 | g7023216 | 1.00E−14 | unnamed protein product (Homo sapiens) |
| 51 | LG:1087707.1:2000FEB18 | g347905 | 2.00E−40 | Human zinc finger protein (ZNF141) mRNA, complete cds. |
| 52 | LG:1090915.1:2000FEB18 | g347905 | 2.00E−24 | Human zinc finger protein (ZNF141) mRNA, complete cds. |
| 53 | LG:1094230.1:2000FEB18 | g454818 | 1.00E−98 | Human Krueppel-related DNA-binding protein (PF4) mRNA, 5′ end. |
| 54 | LG:474848.3:2000FEB18 | g498152 | 4.00E−16 | ha0946 protein is Kruppel-related. |
| 55 | LI:251656.1:2000FEB01 | g55471 | 0 | Zfp-29 |
| 56 | LI:021371.1:2000FEB01 | g984814 | 2.00E−96 | zinc finger protein |
| 57 | LI:133095.1:2000FEB01 | g453376 | 8.00E−42 | zinc finger protein PZF |
| 58 | LI:236654.2:2000FEB01 | g498721 | 2.00E−22 | zinc finger protein |
| 59 | LI:200009.1:2000FEB01 | g498719 | 4.00E−24 | zinc finger protein |
| 60 | LI:758502.1:2000FEB01 | g200407 | 0 | pMLZ-4 |
| 61 | LI:344772.1:2000FEB01 | g4062983 | 3.00E−67 | Eos protein |
| 62 | LI:789445.1:2000FEB01 | g1049301 | 2.00E−26 | KRAB zinc finger protein; Method:conceptual translation supplied by |
| 63 | LI:789657.1:2000FEB01 | g1020145 | 1.00E−53 | DNA binding protein |
| 64 | LI:789808.1:2000FEB01 | g288424 | 0 | Human ZNF37A mRNA for zinc finger protein. |
| 65 | LI:792919.1:2000FEB01 | g2232012 | 0 | Human zinc finger protein (FDZF2) mRNA, complete cds. |
| 66 | LI:793949.1:2000FEB01 | g1017721 | 3.00E−53 | Human repressor transcriptional factor (ZNF85) mRNA, complete cds. |
| 67 | LI:794389.1:2000FEB01 | g5640017 | 4.00E−45 | zinc finger protein ZFP113 |
| 68 | LI:796010.1:2000FEB01 | g288424 | 0 | Human ZNF37A mRNA for zinc finger protein. |
| 69 | LI:796324.1:2000FEB01 | g288424 | 0 | Human ZNF37A mRNA for zinc finger protein. |
| 70 | LI:796373.1:2000FEB01 | g1020145 | 9.00E−36 | DNA binding protein |
| 71 | LI:796415.1:2000FEB01 | g498151 | 4.00E−28 | Human mRNA for KIAA0065gene, partial cds. |
| 72 | LI:798636.1:2000FEB01 | g2970037 | 0 | Human HKL1 mRNA, complete cds. |
| 73 | LI:800045.1:2000FEB01 | g538413 | 2.00E−55 | zinc finger protein |
| 74 | LI:800680.1:2000FEB01 | g7023216 | 7.00E−18 | unnamed protein product (Homo sapiens) |
| 75 | LI:800894.1:2000FEB01 | g3342001 | 0 | Human hematopoietic cell derived zinc finger protein mRNA, complete |
| 76 | LI:801015.1:2000FEB01 | g487785 | 4.00E−16 | zinc finger protein ZNF136 (Homo sapiens) |
| 77 | LI:801236.1:2000FEB01 | g488555 | 4.00E−48 | zinc finger protein ZNF135 |
| 78 | LI:803335.1:2000FEB01 | g498152 | 1.00E−20 | ha0946 protein is Kruppel-related. |
| 79 | U:803998.1:2000FEB01 | g1017722 | 1.00E−53 | repressor transcriptional factor |
| 80 | LI:478757.1:2000FEB01 | g498151 | 9.00E−27 | Human mRNA for KIAA0065 gene, partial cds. |
| 81 | LI:808532.1:2000FEB01 | g2232012 | 0 | Human zinc finger protein (FDZF2) mRNA, complete cds. |
| 82 | LI:443073.1:2000FEB01 | g4567179 | 3.00E−33 | BC37295_1 (Homo sapiens) |
| 83 | LI:479671.1:2000FEB01 | g487784 | 3.00E−38 | Human zinc finger protein ZNF136. |
| 84 | LI:810078.1:2000FEB01 | g498718 | 0 | Human HZF1 mRNA for zinc finger protein. |
| 85 | LI:810224.1:2000FEB01 | g288424 | 0 | Human ZNF37A mRNA for zinc finger protein. |
| 86 | LI:817052.2:2000FEB01 | g1020145 | 1.00E−51 | DNA binding protein |
| 87 | LG:892274.1:2000MAY19 | g6650686 | 2.00E−95 | Human Y-linked zinc finger protein (ZFY) gene, complete cds. |
| 88 | LG:1080959.1:2000MAY19 | g5262560 | 2.00E−40 | hypothetical protein (Homo sapiens) |
| 89 | LG:1054900.1:2000MAY19 | g5262560 | 3.00E−35 | hypothetical protein (Homo sapiens) |
| 90 | LG:1077357.1:2000MAY19 | g10047297 | 2.00E−23 | KIAA1611 protein (Homo sapiens) |
| 91 | LG:1084051.1:2000MAY19 | g5931821 | 8.00E−79 | dJ228H13.3 (zinc finger protein) (Homo sapiens) |
| 92 | LG:1076853.1:2000MAY19 | g506502 | 1.00E−141 | NK10 (Mus musculus) |
| 93 | LG:481631.10:2000MAY19 | g7023216 | 1.00E−142 | unnamed protein product (Homo sapiens) |
| 94 | LG:1088431.2:2000MAY19 | g7023216 | 7.00E−18 | unnamed protein product (Homo sapiens) |
| 95 | LI:401619.10:2000MAY01 | g7959865 | 1.00E−18 | PRO2032 (Homo sapiens) |
| 96 | LI:1144007.1:2000MAY01 | g5360097 | 0 | putative kruppel-related zinc finger protein NY-REN-23 antigen (Homo |
| | | | | sapiens) |
| 97 | LI:331074.1:2000MAY01 | g2149792 | 0 | Roaz (Rattus norvegicus) |
| 98 | LI:1170349.1:2000MAY01 | g487787 | 1.00E−45 | zinc finger protein ZNF140 (Homo sapiens) |
| 99 | LG:335097.1:2000FEB18 | g7020440 | 6.00E−25 | unnamed protein product (Homo sapiens) |
| 100 | LG:1076451.1:2000FEB18 | g2088550 | 0 | Human hereditary haemochromatosis region, histone 2A-like protein |
| | | | | gene, hereditary haemochromatosis (HLA-H) gene, RoRet gene, and |
| | | | | sodium phosphate transporter (NPT3) gene, complete cds. |
| 101 | LI:805478.1:2000FEB01 | g2088550 | 0 | Human hereditary haemochromatosis region, histone 2A-like protein |
| | | | | gene, hereditary haemochromatosis (HLA-H) gene, RoRet gene, and |
| | | | | sodium phosphate transporter (NPT3) gene. complete cds. |
| 102 | LG:101269.1:2000MAY19 | g3953533 | 2.10E−56 | inwardly rectifying potassium channel Klr5.1 |
| 103 | LI:331087.1:2000MAY01 | g4186073 | 3.00E−41 | calcium channel alpha-2-delta-C subunit (Mus musculus) |
| 104 | LI:410188.1:2000MAY01 | g4836145 | 0 | tetrodotoxin-resistant voltage-gated sodium channel (Homo sapiens) |
| 105 | LI:1188288.1:2000MAY01 | g204220 | 0 | beta-alanine-sensitive neuronal GABA transporter (Rattus norvegicus) |
| 106 | LI:427997.4:2000MAY01 | g6996442 | 1.00E−48 | CTL1 protein (Homo sapiens) |
| 107 | LG:451682.1:2000FEB18 | g5091520 | 3.00E−75 | ESTs AU058081 (E30812), AU058365(E50679), AU030138(E50679) |
| | | | | oleracea mRNA for proteasome 37 kD subunit.(X96974) |
| 108 | LG:1077283.1:2000FEB18 | g2565302 | 0 | Rhesus monkey cyclophilin A mRNA, complete cds. |
| 109 | LG: 481436.5:2000FEB18 | g3873707 | 2.60E−34 | Similarity to B. subtilis DNAJ protein (SW: DNAJ_BACSU); cDNA EST |
| | | | | yk437a1.5 comes from this gene |
| 110 | LI:793701.1:2000FEB01 | g1049231 | 4.00E−33 | Method: conceptual translation supplied by author; putative hybrid |
| | | | | protein similar to HERV-H protease and HERV-E integrase (Human |
| | | | | endogenous retrovirus) |
| 111 | LI:373637.1:2000FEB01 | g2286123 | 3.00E−50 | testis specific DNAj-homolog |
| 112 | LG:239368.2:2000MAY19 | g4981382 | 1.00E−11 | dnaJ protein (Thermotoga maritima) |
| 113 | LI:053826.1:2000MAY01 | g2943716 | 4.00E−67 | 25 kDa trypsin inhibitor (Homo sapiens) |
| 114 | LI:449393.1:2000MAY01 | g6957716 | 1.00E−128 | putative chaperonin (Arabidopsis thaliana) |
| 115 | LI:1071427.96:2000MAY01 | g9956070 | 1.00E−144 | similar to Homo sapiens mRNA for KIAA0723 protein with GenBank |
| | | | | Accession Number AB018266.10 |
| 116 | LI:336338.8:2000MAY01 | g9296929 | 2.00E−16 | protease PC6 isoform A (Homo sapiens) |
| 117 | LG:345527.1:2000FEB18 | g805296 | 3.40E−176 | lymphocyte specific helicase |
| 118 | LG:1089383.1:2000FEB18 | g2104910 | 3.00E−23 | ORF derived from D1 leader region and integrase coding region (Homo |
| | | | | sapiens) |
| 119 | LG:1092522.1:2000FEB18 | g1263080 | 0 | Human mariner1 transposase gene, complete consensus sequence. |
| 120 | LG:1093216.1:2000FEB18 | g2104910 | 1.00E−23 | ORF derived from D1 leader region and integrase coding region (Homo |
| | | | | sapiens) |
| 121 | LI:270318.3:2000FEB01 | g3880433 | 3.00E−12 | similar to mitochondrial RNA splicing MSR4 like protein; cDNA EST |
| | | | | EMBL: C09217 comes from this gene |
| 122 | LI:335671.2:2000FEB01 | g805296 | 1.00E−83 | lymphocyte specific helicase |
| 123 | LI:793758.1:2000FEB01 | g2104910 | 4.00E−26 | ORF derived from D1 leader region and integrase coding region (Homo |
| | | | | sapiens) |
| 124 | LI:803718.1:2000FEB01 | g2104910 | 3.00E−23 | ORF derived from D1 leader region and integrase coding region (Homo |
| | | | | sapiens) |
| 125 | LI:412179.1:2000FEB01 | g1263080 | 4.00E−93 | Human mariner1 transposase gene, complete consensus sequence. |
| 126 | LI:815679.1:2000FEB01 | g7020440 | 3.00E−12 | unnamed protein product (Homo sapiens) |
| 127 | LI:481361.3:2000FEB01 | g3776011 | 5.00E−25 | RNA helicase |
| 128 | LG:247388.1:2000MAY19 | g6016932 | 3.00E−127 | dJ620E11.1a (novel Helicase C-terminal domain and SNF2 N-terminal |
| | | | | domains containing protein, similar to KIAA0308) |
| 129 | LG:255789.10:2000MAY19 | g37542 | 2.00E−57 | Human mRNA for U1 small nuclear RNP-specific C protein. |
| 130 | LI:787618.1:2000MAY01 | g7020440 | 3.00E−12 | unnamed protein product (Homo sapiens) |
| 131 | LI:331610.2:2000MAY01 | g2599502 | 0 | protocadherin 68 (Homo sapiens) |
| 132 | LG:982697.1:2000FEB18 | g10436424 | 1.00E−25 | unnamed protein product (Homo sapiens) |
| 133 | LG:1080896.1:2000FEB18 | g5926696 | 0 | Human genomic DNA, chromosome 6p21.3, HLA Class I region, section |
| | | | | 8/20. |
| 134 | LI:811341.1:2000FEB01 | g5926696 | 0 | Human genomic DNA, chromosome 6p21.3, HLA Class I region, section |
| | | | | 8/20. |
| 135 | LI:903225.1:2000FEB01 | g5926710 | 0 | Human genomic DNA, chromosome 6p21.3, HLA Class I region, section |
| | | | | 20/20. |
| 136 | LI:242079.2:2000FEB01 | g5926703 | 0 | Human genomic DNA, chromosome 6p21.3, HLA Class I region, section |
| | | | | 15/20. |
| 137 | LG:979580.1:2000MAY19 | g9280152 | 7.00E−23 | unnamed portein product (Macaca fascicularis) |
| 138 | LI:1169865.1:2000MAY01 | g673417 | 1.00E−112 | class II antigen (Homo sapiens) |
| 139 | LG:337818.2:2000FEB18 | g404777 | 4.80E−84 | cytochrome P-450 2B-Bx |
| 140 | LI:337818.1:2000FEB01 | g203759 | 4.00E−58 | cytochrome P-450(1) |
| 141 | LG:241577.4:2000MAY19 | g2809498 | 1.50E−29 | cytochrome c oxidase subunit IV |
| 142 | LG:344786.4:2000MAY19 | g164981 | 2.00E−06 | cytochrome P-450p-2 (Oryctolagus cuniculus) |
| 143 | LI:414307.1:2000FEB01 | g30095 | 9.00E−48 | collagen subunit (alpha-1 (X)) 3 |
| 144 | LI:202943.2:2000FEB01 | g391663 | 7.00E−06 | hikaru genki type 1 product |
| 145 | LI:246194.2:2000FEB01 | g1405821 | 1.00E−05 | SULFATED SURFACE GLYCOPROTEIN 185 |
| 146 | LI:815961.1:2000FEB01 | g292045 | 0 | Human mucin mRNA, partial cds. |
| 147 | LG:120744.1:2000MAY19 | g4582324 | 1.00E−168 | dJ708F5.1 (PUTATIVE novel Collagen alpha 1 LIKE protein) (Homo |
| 148 | LI:757520.1:2000MAY01 | g7161771 | 0 | keratin (Homo sapiens) |
| 149 | LG:160570.1:2000FEB18 | g466548 | 1.00E−46 | NBL4 |
| 150 | LI:350398.3:2000FEB01 | g3724141 | 1.00E−06 | myosin I |
| 151 | LI:221285.1:2000FEB01 | g18218 | 2.00E−74 | spoke protein |
| 152 | LI:401605.2:2000FEB01 | g1755049 | 1.00E−15 | myosin X |
| 153 | LI:329017.1:2000FEB01 | g1813638 | 2.00E−51 | PF20 |
| 154 | LI:401322.1:2000FEB01 | g38076 | 2.00E−30 | Macaque mRNA for alpha-tubulin. |
| 155 | LG:403409.1:2000MAY19 | g7303061 | 0 | Khc-73 gene product (Drosophila melanogaster) |
| 156 | LG:233933.5:2000MAY19 | g7385113 | 2.00E−18 | ankyrin 1 (Bos taurus) |
| 157 | LI:290344.1:2000MAY01 | g1353782 | 0 | dystrophin-related protein 2 (Homo sapiens) |
| 158 | LI:410742.1:2000MAY01 | g2290200 | 0 | desmoglein 3 (Mus musculus) |
| 159 | LG:406568.1:2000MAY19 | g28969 | 5.30E−44 | 64 Kd autoantigen |
| 160 | LI:283762.1:2000MAY01 | g1469868 | 0 | The KIAA0143 gene product is related to a putative C. elegans gene |
| | | | | encoded on cosmid C32D5. (Homo sapiens) |
| 161 | LI:347687.113:2000MAY01 | g387514 | 1.00E−123 | DM-20 protein (Mus musculus) |
| 162 | LI:1146510.1:2000MAY01 | g2149291 | 3.00E−24 | defender against death 1 protein (Homo sapiens) |
| 163 | LG:451710.1:2000FEB18 | g5816996 | 2.40E−42 | ribosomal protein L32-like protein |
| 164 | LG:455771.1:2000FEB18 | g643074 | 1.70E−59 | putative 40S ribosomal protein s12 |
| 165 | LG:452089.1:2000FEB18 | g463252 | 1.90E−62 | RL5 ribosomal protein |
| 166 | LG:246415.1:2000FEB18 | g296451 | 0 | Human mRNA for ribosomal protein S26. |
| 167 | LG:414144.10:2000FEB18 | g200785 | 1.80E−16 | ribosomal protein L7 |
| 168 | LG:1101445.1:2000FEB18 | g1800114 | 0 | Human ribosomal protein L7 antisense mRNA gene, partial sequence. |
| 169 | LG:452134.1:2000FEB18 | g550024 | 0 | Human ribosomal protein S10 mRNA, complete cds. |
| 170 | LI:903021.1:2000FEB01 | g36139 | 0 | Human mRNA for ribosomal protein L7. |
| 171 | LI:246422.1:2000FEB01 | g409069 | 0 | Human mRNA for HBp15/L22, complete cds. |
| 172 | LG:449404.1:2000MAY19 | g4886269 | 5.00E−66 | putative ribosomal protein S14 (Arabidopsis thaliana) |
| 173 | LG:449413.1:2000MAY19 | g643074 | 1.00E−70 | putative 40S ribosomal protein s12 (Fragaria x ananassa) |
| 174 | LG:450105.1:2000MAY19 | g643074 | 6.00E−76 | putative 40S ribosomal protein s12 (Fragaria x ananassa) |
| 175 | LG:460809.1:2000MAY19 | g36129 | 4.00E−54 | Human mRNA for ribosomal protein L31. |
| 176 | LG:481781.1:2000MAY19 | g2331301 | 1.00E−130 | ribosomal protein S4 type I (Zea mays) |
| 177 | LG:1101153.1:2000MAY19 | g2668748 | 2.00E−95 | ribosomal protein L17 (Zea mays) |
| 178 | LI:257695.20:2000MAY01 | g57714 | 1.00E−62 | ribosomal protein S16 (AA 1-146) (Rattus rattus) |
| 179 | LI:455771.1:2000MAY01 | g643074 | 6.00E−76 | putative 40S ribosomal protein s12 (Fragaria x ananassa) |
| 180 | LI:274551.1:2000MAY01 | g36145 | 2.00E−59 | Human mRNA for ribosomal protein S12. |
| 181 | LI:035973.1:2000MAY01 | g57121 | 8.00E−29 | ribosomal protein L37 (Rattus norvegicus) |
| 182 | LG:978427.5:2000FEB18 | g545998 | 2.50E−67 | tricarboxylate carrier (rats, liver, Peptide Mitochondrial Partial, 357 aa) |
| 183 | LG:247781.2:2000FEB18 | g2352427 | 9.40E−29 | peroxisomal Ca-dependent solute carrier |
| 184 | LI:034583.1:2000FEB01 | g5815141 | 0 | nuclear body associated kinase 1b |
| 185 | LI:333307.2:2000FEB01 | g295671 | 0.0003 | selected as a weak suppressor of a mutant of the subunit AC40 of DNA |
| | | | | dependant RNA polymerase I and III |
| 186 | LI:814710.2:2000FEB01 | g178281 | 1.00E−46 | AHNAK nucleoprotein |
| 187 | LG:414732.1:2000MAY19 | g183233 | 2.00E−54 | beta-glucuronidase precursor (EC 3.2.1.31) |
| 188 | LG:413910.6:2000MAY19 | g7022046 | 1.00E−109 | unnamed protein product (Homo sapiens) |
| 189 | LI:414732.2:2000MAY01 | g183232 | 0 | Human beta-glucuronidase mRNA, complete cds. |
| 190 | LI:900264.2:2000MAY01 | g414797 | 2.00E−81 | pyruvate dehydrogenase phosphatase (Bos taurus) |
| 191 | LI:335593.1:2000MAY01 | g3851553 | 5.00E−34 | RNA-binding protein Nova-2 (Homo sapiens) |
| 192 | LI:1189543.1:2000MAY01 | g7025507 | 0 | ventral neuron-specific protein 1 NOVA1 (Mus musculus) |
| 193 | LG:455450.1:2000FEB18 | g4105111 | 2.10E−20 | dehydrin 6 |
| 194 | LG:1040978.1:2000FEB18 | g453189 | 3.30E−41 | acyl carrier protein |
| 195 | LG:446649.1:2000FEB18 | g181960 | 2.00E−35 | Human endozepine (putative ligand of benzodiazepine receptor) |
| | | | | mRNA, complete cds. |
| 196 | LG:132147.3:2000FEB18 | g6446606 | 0 | E3 ubiquitin ligase SMURF1 (Homo sapiens) |
| 197 | LI:036034.1:2000FEB01 | g9622856 | 1.00E−33 | sorting nexin 15A (Homo sapiens) |
| 198 | LG:162161.1:2000MAY19 | g5823961 | 3.00E−87 | dJ20B11.1 (ortholog of rat RSEC5 (mammalian exocyst complex subunit)) |
| | | | | (Homo sapiens) |
| 199 | LG:407214.10:2000MAY19 | g9963839 | 4.00E−54 | lipase (Homo sapiens) |
| 200 | LG:204626.1:2000MAY19 | g3243240 | 5.10E−41 | syntaxin 11 |
| 201 | LI:007401.1:2000MAY01 | g4512103 | 3.00E−81 | rab 11 binding protein (Bos taurus) |
| 202 | LI:476342.1:2000MAY01 | g790641 | 2.00E−21 | gamma-thionin (Hordeum vulgare) |
| 203 | LI:1072759.1:2000MAY01 | g2367625 | 4.00E−21 | protein synthesis elongation factor 1-alpha (Rhodotorula mucilaginosa) |
| 204 | LG:998857.1:2000FEB18 | g2731641 | 6.10E−13 | Fas-ligand associated factor 3 |
| 205 | LG:482261.1:2000FEB18 | g4003386 | 0 | Human genomic DNA of 8p21.3-p22 anti-oncogene of hepatocellular |
| | | | | colorectal and non-small cell lung cancer, segment 9/11. |
| 206 | LG:480328.1:2000FEB18 | g246482 | 0 | prohibitin (Human, mRNA, 1043 nt). |
| 207 | LG:311197.1:2000MAY19 | g505033 | 8.00E−62 | mitogen inducible gene mig-2 (Homo sapiens) |
| 208 | LG:1054883.1:2000MAY19 | g325464 | 0 | Human endogenous retrovirus type C oncovirus sequence. |
| 209 | LG:399395.1:2000MAY19 | g1177607 | 8.00E−10 | pva1 (Plasmodium vivax) |
| 210 | LG:380497.2:2000MAY19 | g10504238 | 3.00E−88 | hepatocellular carcinoma-related putative tumor suppressor (Homo |
| 211 | LI:272913.22:2000MAY01 | g4982485 | 3.00E−59 | apoptosis related protein APR-3 (Homo sapiens) |

TABLE 2

| SEQ ID NO: | Template ID | Start | Stop | Frame | Pfam Hit | Pfam Description | E-value |
|---|---|---|---|---|---|---|---|
| 1 | LG:1040582.1:2000FEB18 | 267 | 539 | forward 3 | aldo_ket_red | Aldo/keto reductase family | 2.50E−51 |
| 2 | LG:453570.1:2000FEB18 | 186 | 605 | forward 3 | Glyoxalase | Glyoxalase | 3.80E−72 |
| 3 | LG:408751.3:2000FEB18 | 194 | 1345 | forward 2 | Dihydrooratase | Dihydroorotase-like | 1.40E−19 |
| 4 | LI:090574.1:2000FEB01 | 60 | 776 | forward 3 | carb_anhydrase | Eukaryotic-type carbonic anhydrase | 9.70E−144 |
| 6 | LI:332176.1:2000FEB01 | 2 | 961 | forward 2 | Glyco_hydro_31 | Glycosyl hydrolases family 31 | 4.10E−144 |
| 7 | LI:403248.2:2000FEB01 | 191 | 367 | forward 2 | Orn_DAP_Arg_deC | Pyrldoxal-dependent decarboxylase | 1.40E−12 |
| 8 | LG:220992.1:2000MAY19 | 156 | 1556 | forward 3 | Amidase | Amidase | 1.10E−153 |
| 9 | LG:1094571.1:2000MAY19 | 328 | 720 | forward 1 | FAD_Synth | Riboflavin kinase/FAD synthetase | 2.30E−42 |
| 10 | LI:350754.4:2000MAY01 | 855 | 1121 | forward 3 | Transglut_core | Transglutaminase-like superfamily | 2.90E−47 |
| 10 | LI:350754.4:2000MAY01 | 1455 | 2132 | forward 3 | Transglutamin_C | Transglutaminase family | 3.20E−106 |
| 10 | LI:350754.4:2000MAY01 | 54 | 413 | forward 3 | Transglutamin_N | Transglutaminase family | 2.50E−63 |
| 11 | LI:255828.29:2000MAY01 | 2 | 367 | forward 2 | PK | Pyruvate kinase | 7.00E−71 |
| 11 | LI:255828.29:2000MAY01 | 348 | 512 | forward 3 | PK | Pyruvate kinase | 5.70E−24 |
| 12 | LI:1190263.1:2000MAY01 | 281 | 1750 | forward 2 | Sulfatase | Sulfatase | 8.60E−66 |
| 14 | LG:999414.3:2000FEB18 | 718 | 1038 | forward 1 | 7tm_1 | 7 transmembrane receptor | 3.60E−13 |
| | | | | | | (rhodopsin family) | |
| 14 | LG:999414.3:2000FEB18 | 1115 | 1453 | forward 2 | 7tm_1 | 7 transmembrane receptor | 4.30E−07 |
| | | | | | | (rhodopsin family) | |
| 18 | LG:068682.1:2000FEB18 | 176 | 883 | forward 2 | pkinase | Eukaryotic protein kinase domain | 1.70E−65 |
| 19 | LG:242665.1:2000FEB18 | 190 | 747 | forward 1 | ras | Ras family | 2.30E−34 |
| 20 | LG:241743.1:2000FEB18 | 199 | 345 | forward 1 | PH | PH domain | 8.00E−06 |
| 22 | LG:344886.1:2000MAY19 | 379 | 957 | forward 1 | ras | Ras family | 1.70E−17 |
| 25 | LG:898771.1:2000MAY19 | 525 | 662 | forward 3 | Fork_head | Fork head domain | 1.70E−25 |
| 28 | LI:1085273.2:2000MAY01 | 285 | 1070 | forward 3 | DSPc | Dual specificity phosphatase, | 1.30E−39 |
| | | | | | | catalytic domain | |
| 29 | LI:333138.2:2000MAY01 | 291 | 1016 | forward 3 | pkinase | Eukaryotic protein kinase domain | 2.00E−90 |
| 32 | LG:998283.7:2000FEB18 | 370 | 630 | forward 1 | bromodomain | Bromodomain | 2.60E−29 |
| 32 | LG:998283.7:2000FEB18 | 4 | 153 | forward 1 | PHD | PHD-finger | 1.90E−12 |
| 34 | LI:175223.1:2000FEB01 | 210 | 431 | forward 3 | CSD | ‘Cold-shock’ DNA-binding domair | 1.40E−18 |
| 38 | LG:021371.3:2000FEB18 | 932 | 1000 | forward 2 | zf-C2H2 | Zinc finger, C2H2 type | 2.10E−04 |
| 39 | LG:475404.1:2000FEB18 | 176 | 328 | forward 2 | KRAB | KRAB box | 1.10E−15 |
| 40 | LG:979406.2:2000FEB18 | 85 | 273 | forward 1 | KRAB | KRAB box | 2.40E−34 |
| 41 | LG:410726.1:2000FEB18 | 646 | 834 | forward 1 | KRAB | KRAB box | 2.10E−17 |
| 41 | LG:410726.1:2000FEB18 | 274 | 558 | forward 1 | SCAN | SCAN domain | 8.90E−55 |
| 43 | LG:1076828.1:2000FEB18 | 448 | 516 | forward 1 | zf-C2H2 | Zinc finger, C2H2 type | 2.00E−07 |
| 44 | LG:1076931.1:2000FEB18 | 173 | 310 | forward 2 | KRAB | KRAB box | 3.40E−21 |
| 45 | LG:1078121.1:2000FEB18 | 186 | 374 | forward 3 | KRAB | KRAB box | 2.80E−41 |
| 46 | LG:1079203.1:2000FEB18 | 421 | 489 | forward 1 | zf-C2H2 | Zinc finger, C2H2 type | 6.00E−06 |
| 46 | LG:1079203.1:2000FEB18 | 647 | 715 | forward 2 | zf-C2H2 | Zinc finger, C2H2 type | 9.20E−05 |
| 47 | LG:1082586.1:2000FEB18 | 414 | 536 | forward 3 | KRAB | KRAB box | 6.80E−12 |
| 48 | LG:1082774.1:2000FEB18 | 138 | 326 | forward 3 | KRAB | KRAB box | 1.30E−40 |
| 49 | LG:1082775.1:2000FEB18 | 45 | 230 | forward 3 | KRAB | KRAB box | 7.10E−39 |
| 49 | LG:1082775.1:2000FEB18 | 840 | 908 | forward 3 | zf-C2H2 | Zinc finger, C2H2 type | 4.40E−05 |
| 50 | LG:1083120.1:2000FEB18 | 117 | 266 | forward 3 | KRAB | KRAB box | 5.10E−22 |
| 51 | LG:1087707.1:2000FEB18 | 162 | 350 | forward 3 | KRAB | KRAB box | 2.80E−40 |
| 52 | LG:1090915.1:2000FEB18 | 129 | 251 | forward 3 | KRAB | KRAB box | 7.40E−22 |
| 53 | LG:1094230.1:2000FEB18 | 120 | 308 | forward 3 | KRAB | KRAB box | 3.70E−41 |
| 54 | LG:474848.3:2000FEB18 | 253 | 441 | forward 1 | KRAB | KRAB box | 2.10E−38 |
| 55 | LI:251656.1:2000FEB01 | 242 | 310 | forward 2 | zf-C2H2 | Zinc finger, C2H2 type | 3.90E−08 |
| 56 | LI:021371.1:2000FEB01 | 717 | 785 | forward 3 | zf-C2H2 | Zinc finger, C2H2 type | 2.10E−04 |
| 57 | LI:133095.1:2000FEB01 | 539 | 607 | forward 2 | zf-C2H2 | Zinc finger, C2H2 type | 4.30E−06 |
| 58 | LI:236654.2:2000FEB01 | 805 | 873 | forward 1 | zf-C2H2 | Zinc finger, C2H2 type | 1.40E−04 |
| 59 | LI:200009.1:2000FEB01 | 564 | 632 | forward 3 | zf-C2H2 | Zinc finger, C2H2 type | 1.80E−05 |
| 60 | LI:758502.1:2000FEB01 | 633 | 701 | forward 3 | zf-C2H2 | Zinc finger, C2H2 type | 2.50E−07 |
| 62 | LI:789445.1:2000FEB01 | 71 | 262 | forward 2 | KRAB | KRAB box | 1.60E−27 |
| 63 | LI:789657.1:2000FEB01 | 542 | 610 | forward 2 | zf-C2H2 | Zinc finger, C2H2 type | 2.60E−06 |
| 64 | LI:789808.1:2000FEB01 | 272 | 340 | forward 2 | zf-C2H2 | Zinc finger, C2H2 type | 1.00E−07 |
| 64 | LI:789808.1:2000FEB01 | 426 | 494 | forward 3 | zf-C2H2 | Zinc finger, C2H2 type | 3.40E−04 |
| 65 | LI:792919.1:2000FEB01 | 31 | 99 | forward 1 | zf-C2H2 | Zinc finger, C2H2 type | 5.30E−06 |
| 66 | LI:793949.1:2000FEB01 | 120 | 308 | forward 3 | KRAB | KRAB box | 1.70E−41 |
| 67 | LI:794389.1:2000FEB01 | 75 | 143 | forward 3 | zf-C2H2 | Zinc finger, C2H2 type | 8.70E−06 |
| 68 | LI:796010.1:2000FEB01 | 276 | 344 | forward 3 | zf-C2H2 | Zinc finger, C2H2 type | 1.00E−07 |
| 68 | LI:796010.1:2000FEB01 | 433 | 501 | forward 1 | zf-C2H2 | Zinc finger, C2H2 type | 3.40E−04 |
| 69 | LI:796324.1:2000FEB01 | 290 | 358 | forward 2 | zf-C2H2 | Zinc finger, C2H2 type | 1.00E−07 |
| 69 | LI:796324.1:2000FEB01 | 450 | 518 | forward 3 | zf-C2H2 | Zinc finger, C2H2 type | 3.40E−04 |
| 70 | LI:796373.1:2000FEB01 | 181 | 249 | forward 1 | zf-C2H2 | Zinc finger, C2H2 type | 1.10E−06 |
| 71 | LI:796415.1:2000FEB01 | 45 | 230 | forward 3 | KRAB | KRAB box | 7.10E−39 |
| 72 | LI:798636.1:2000FEB01 | 329 | 397 | forward 2 | zf-C2H2 | Zinc finger, C2H2 type | 2.60E−07 |
| 73 | LI:800045.1:2000FEB01 | 364 | 432 | forward 1 | zf-C2H2 | Zinc finger, C2H2 type | 5.30E−07 |
| 74 | LI:800680.1:2000FEB01 | 155 | 319 | forward 2 | KRAB | KRAB box | 5.00E−21 |
| 75 | LI:800894.1:2000FEB01 | 125 | 313 | forward 2 | KRAB | KRAB box | 6.50E−40 |
| 76 | LI:801015.1:2000FEB01 | 22 | 216 | forward 1 | KRAB | KRAB box | 3.00E−24 |
| 77 | LI:801236.1:2000FEB01 | 225 | 293 | forward 3 | zf-C2H2 | Zinc finger, C2H2 type | 4.40E−07 |
| 78 | LI:803335.1:2000FEB01 | 220 | 408 | forward 1 | KRAB | KRAB box | 2.10E−38 |
| 79 | LI:803998.1:2000FEB01 | 62 | 130 | forward 2 | zf-C2H2 | Zinc finger, C2H2 type | 1.20E−05 |
| 80 | LI:478757.1:2000FEB01 | 467 | 643 | forward 2 | KRAB | KRAB box | 2.40E−21 |
| 81 | LI:808532.1:2000FEB01 | 53 | 121 | forward 2 | zf-C2H2 | Zinc finger, C2H2 type | 5.70E−05 |
| 82 | LI:443073.1:2000FEB01 | 176 | 244 | forward 2 | zf-C2H2 | Zinc finger, C2H2 type | 2.50E−05 |
| 83 | LI:479671.1:2000FEB01 | 160 | 312 | forward 1 | KRAB | KRAB box | 1.70E−19 |
| 84 | LI:810078.1:2000FEB01 | 424 | 492 | forward 1 | zf-C2H2 | Zinc finger, C2H2 type | 1.80E−06 |
| 84 | LI:810078.1:2000FEB01 | 587 | 655 | forward 2 | zf-C2H2 | Zinc finger, C2H2 type | 1.20E−05 |
| 85 | LI:810224.1:2000FEB01 | 171 | 239 | forward 3 | zf-C2H2 | Zinc finger, C2H2 type | 1.00E−07 |
| 86 | LI:817052.2:2000FEB01 | 901 | 969 | forward 1 | zf-C2H2 | Zinc finger, C2H2 type | 8.90E−08 |
| 87 | LG:892274.1:2000MAY19 | 96 | 461 | forward 3 | dUTPase | dUTPase | 9.20E−27 |
| 87 | LG:892274.1:2000MAY19 | 489 | 752 | forward 3 | rvp | Retroviral aspartyl protease | 5.30E−11 |
| 88 | LG:1080959.1:2000MAY19 | 182 | 322 | forward 2 | KRAB | KRAB box | 2.00E−16 |
| 89 | LG:1054900.1:2000MAY19 | 78 | 218 | forward 3 | KRAB | KRAB box | 2.30E−17 |
| 90 | LG:1077357.1:2000MAY19 | 94 | 282 | forward 1 | KRAB | KRAB box | 4.80E−31 |
| 91 | LG:1084051.1:2000MAY19 | 195 | 263 | forward 3 | zf-C2H2 | Zinc finger, C2H2 type | 1.80E−06 |
| 92 | LG:1076853.1:2000MAY19 | 706 | 774 | forward 1 | zf-C2H2 | Zinc finger, C2H2 type | 1.50E−07 |
| 93 | LG:481631.10:2000MAY19 | 96 | 263 | forward 3 | KRAB | KRAB box | 5.70E−25 |
| 93 | LG:481631.10:2000MAY19 | 882 | 950 | forward 3 | zf-C2H2 | Zinc finger, C2H2 type | 1.70E−05 |
| 94 | LG:1088431.2:2000MAY19 | 175 | 339 | forward 1 | KRAB | KRAB box | 5.00E−21 |
| 96 | LI:1144007.1:2000MAY01 | 914 | 1108 | forward 2 | KRAB | KRAB box | 5.90E−05 |
| 96 | LI:1144007.1:2000MAY01 | 323 | 610 | forward 2 | SCAN | SCAN domain | 4.10E−60 |
| 97 | LI:331074.1:2000MAY01 | 194 | 262 | forward 2 | zf-C2H2 | Zinc finger, C2H2 type | 1.00E−03 |
| 98 | LI:1170349.1:2000MAY01 | 185 | 370 | forward 2 | KRAB | KRAB box | 2.50E−29 |
| 98 | LI:1170349.1:2000MAY01 | 740 | 808 | forward 2 | zf-C2H2 | Zinc finger, C2H2 type | 5.80E−05 |
| 102 | LG:101269.1:2000MAY19 | 556 | 831 | forward 1 | IRK | Inward rectifier potassium channel | 3.50E−65 |
| 104 | LI:410188.1:2000MAY01 | 3760 | 4569 | forward 1 | ion_trans | Ion transport protein | 3.70E−97 |
| 104 | LI:410188.1:2000MAY01 | 4586 | 5314 | forward 2 | ion_trans | Ion transport protein | 3.30E−66 |
| 105 | LI:1188288.1:2000MAY01 | 751 | 1215 | forward 1 | SNF | Sodium:neurotransmitter | 8.50E−113 |
| | | | | | | symporter family | |
| 105 | LI:1188288.1:2000MAY01 | 423 | 782 | forward 3 | SNF | Sodium:neurotransmitter | 8.60E−74 |
| | | | | | | symporter family | |
| 105 | LI:1188288.1:2000MAY01 | 1187 | 1438 | forward 2 | SNF | Sodium:neurotransmitter | 5.50E−52 |
| | | | | | | symporter family | |
| 107 | LG:451682.1:2000FEB18 | 117 | 560 | forward 3 | proteasome | Proteasome A-type and B-type | 4.40E−59 |
| 108 | LG:1077283.1:2000FEB18 | 110 | 427 | forward 2 | pro_isomerase | Cyclophilin type peptidyl-prolyl | 1.80E−37 |
| | | | | | | cis-trans isomerase | |
| 108 | LG:1077283.1:2000FEB18 | 177 | 278 | forward 3 | pro_isomerase | Cyclophilin type peptidyl-prolyl | 1.30E−18 |
| | | | | | | cis-trans isomerase | |
| 109 | LG:481436.5:2000FEB18 | 351 | 539 | forward 3 | Dnaj | Dnaj domain | 2.80E−28 |
| 111 | LI:373637.1:2000FEB01 | 17 | 217 | forward 2 | Dnaj | Dnaj domain | 6.30E−39 |
| 113 | LI:053826.1:2000MAY01 | 834 | 1106 | forward 3 | SCP | SCP-like extracellular protein | 1.10E−17 |
| 114 | LI:449393.1:2000MAY01 | 90 | 788 | forward 3 | cpn60_TCP1 | TCP-1/cpn60 chaperonin family | 9.80E−66 |
| 117 | LG:345527.1:2000FEB18 | 667 | 957 | forward 1 | helicase_C | Helicases conserved C-terminal domain | 7.20E−21 |
| 117 | LG:345527.1:2000FEB18 | 8 | 631 | forward 2 | SNF2_N | SNF2 and others N-terminal domain | 7.20E−44 |
| 122 | LI:335671.2:2000FEB01 | 188 | 475 | forward 2 | helicase_C | Heilcases conserved C-terminal domain | 9.10E−13 |
| 122 | LI:335671.2:2000FEB01 | 3 | 95 | forward 3 | SNF2_N | SNF2 and others N-terminal domain | 7.10E−06 |
| 128 | LG:247388.1:2000MAY19 | 346 | 600 | forward 1 | helicase_C | Heilcases conserved C-terminal domain | 2.70E−19 |
| 128 | LG:247388.1:2000MAY19 | 3 | 173 | forward 3 | SNF2_N | SNF2 and others N-terminal domain | 1.60E−14 |
| 131 | LI:331610.2:2000MAY01 | 1415 | 1699 | forward 2 | cadherin | Cadherin domain | 6.00E−20 |
| 135 | LI:903225.1:2000FEB01 | 603 | 764 | forward 3 | Ribosomal_L23 | Ribosomal protein L23 | 4.80E−14 |
| 138 | LI:1169865.1:2000MAY01 | 593 | 790 | forward 2 | ig | Immunoglobulin domain | 2.30E−08 |
| 138 | LI:1169865.1:2000MAY01 | 242 | 547 | forward 2 | MHC_II_alpha | Class II histocompatibility antigen, | 1.80E−65 |
| | | | | | | alpha domain | |
| 139 | LG:337818.2:2000FEB18 | 136 | 1518 | forward 1 | p450 | Cytochrome P450 | 1.50E−173 |
| 140 | LI:337818.1:2000FEB01 | 654 | 998 | forward 3 | p450 | Cytochrome P450 | 3.50E−45 |
| 140 | LI:337818.1:2000FEB01 | 136 | 384 | forward 1 | p450 | Cytochrome P450 | 4.40E−27 |
| 140 | LI:337818.1:2000FEB01 | 359 | 673 | forward 2 | p450 | Cytochrome P450 | 5.40E−27 |
| 143 | LI:414307.1:2000FEB01 | 590 | 964 | forward 2 | C1q | C1q domain | 2.30E−38 |
| 143 | LI:414307.1:2000FEB01 | 365 | 544 | forward 2 | Collagen | Collagen triple helix repeat (20 copies) | 2.50E−10 |
| 144 | LI:202943.2:2000FEB01 | 36 | 209 | forward 3 | sushi | Sushi domain (SCR repeat) | 1.40E−09 |
| 147 | LG:120744.1:2000MAY19 | 301 | 813 | forward 1 | vwa | von Willebrand factor type A domain | 2.00E−51 |
| 148 | LI:757520.1:2000MAY01 | 427 | 1362 | forward 1 | filament | Intermediate filament proteins | 7.10E−157 |
| 149 | LG:160570.1:2000FEB18 | 260 | 562 | forward 2 | Band_41 | FERM domain (Band 4.1 family) | 1.60E−22 |
| 152 | LI:401605.2:2000FEB01 | 1 | 129 | forward 1 | myosin_head | Myosin head (motor domain) | 5.90E−07 |
| 153 | LI:329017.1:2000FEB01 | 226 | 336 | forward 1 | WD40 | WD domain, G-beta repeat | 5.10E−06 |
| 154 | LI:401322.1:2000FEB01 | 156 | 341 | forward 3 | tubulin | Tubulin/FtsZ family | 7.10E−20 |
| 154 | LI:401322.1:2000FEB01 | 371 | 478 | forward 2 | tubulin | Tubulin/FtsZ family | 2.50E−06 |
| 155 | LG:403409.1:2000MAY19 | 1458 | 1652 | forward 3 | FHA | FHA domain | 3.00E−04 |
| 155 | LG:403409.1:2000MAY19 | 78 | 1193 | forward 3 | kinesin | Kinesin motor domain | 6.80E−172 |
| 156 | LG:233933.5:2000MAY19 | 258 | 356 | forward 3 | ank | Ank repeat | 4.90E−06 |
| 157 | LI:290344.1:2000MAY01 | 992 | 1312 | forward 2 | spectrin | Spectrin repeat | 4.10E−07 |
| 157 | LI:290344.1:2000MAY01 | 1361 | 1450 | forward 2 | WW | WW domain | 5.40E−08 |
| 158 | LI:410742.1:2000MAY01 | 599 | 889 | forward 2 | cadherin | Cadherin domain | 1.80E−21 |
| 158 | LI:410742.1:2000MAY01 | 1224 | 1520 | forward 3 | cadherin | Cadherin domain | 9.90E−04 |
| 161 | LI:347687.113:2000MAY01 | 214 | 855 | forward 1 | Myelin_PLP | Myelin proteolipid protein | 7.10E−160 |
| | | | | | | (PLP or lipophilin) | |
| 163 | LG:451710.1:2000FEB18 | 130 | 459 | forward 1 | Ribosomal_L32e | Ribosomal protein L32 | 4.80E−57 |
| 164 | LG:455771.1:2000FEB18 | 69 | 473 | forward 3 | Ribosomal_S12 | Ribosomal protein S12 | 6.60E−78 |
| 165 | LG:452089.1:2000FEB18 | 107 | 268 | forward 2 | Ribosomal_L5 | Ribosomal protein L5 | 2.40E−25 |
| 165 | LG:452089.1:2000FEB18 | 278 | 577 | forward 2 | Ribosomal_L5_C | ribosomal L5P family C-terminus | 2.70E−60 |
| 166 | LG:246415.1:2000FEB18 | 27 | 365 | forward 3 | Ribosomal_S26e | Ribosomal protein S26e | 2.40E−59 |
| 168 | LG:1101445.1:2000FEB18 | 306 | 464 | forward 3 | Ribosomal_L30 | Ribosomal protein L30p/L7e | 4.20E−28 |
| 171 | LI:246422.1:2000FEB01 | 53 | 397 | forward 2 | Ribosomal_L22e | Ribosomal L22e protein family | 4.30E−28 |
| 171 | LI:246422.1:2000FEB01 | 64 | 318 | forward 1 | Ribosomal_L22e | Ribosomal L22e protein family | 7.70E−07 |
| 172 | LG:449404.1:2000MAY19 | 175 | 531 | forward 1 | Ribosomal_S11 | Ribosomal protein S11 | 6.90E−77 |
| 173 | LG:449413.1:2000MAY19 | 99 | 368 | forward 3 | Ribosomal_S12 | Ribosomal protein S12 | 6.10E−47 |
| 173 | LG:449413.1:2000MAY19 | 367 | 504 | forward 1 | Ribosomal_S12 | Ribosomal protein S12 | 3.20E−21 |
| 174 | LG:450105.1:2000MAY19 | 86 | 490 | forward 2 | Ribosomal_S12 | Ribosomal protein S12 | 6.60E−78 |
| 175 | LG:460809.1:2000MAY19 | 3 | 236 | forward 3 | Ribosomal_L31e | Ribosomal protein L31e | 6.00E−17 |
| 176 | LG:481781.1:2000MAY19 | 243 | 671 | forward 3 | Ribosomal_S4e | Ribosomal family S4e | 1.40E−97 |
| 177 | LG:1101153.1:2000MAY19 | 89 | 499 | forward 2 | Ribosomal_L22 | Ribosomal protein L22p/L17e | 5.20E−76 |
| 178 | LI:257695.20:2000MAY01 | 110 | 673 | forward 2 | Ribosomal_S9 | Ribosomal protein S9/S16 | 1.60E−40 |
| 179 | LI:455771.1:2000MAY01 | 69 | 473 | forward 3 | Ribosomal_S12 | Ribosomal protein S12 | 6.60E−78 |
| 181 | LI:035973.1:2000MAY01 | 318 | 479 | forward 3 | Ribosomal_L37e | Ribosomal protein L37e | 1.60E−13 |
| 183 | LG:247781.2:2000FEB18 | 142 | 426 | forward 1 | mito_carr | Mitochondrial carrier proteins | 1.80E−24 |
| 190 | LI:900264.2:2000MAY01 | 1151 | 1555 | forward 2 | PP2C | Protein phosphatase 2C | 2.90E−11 |
| 192 | LI:1189543.1:2000MAY01 | 1292 | 1447 | forward 2 | KH-domain | KH domain | 2.50E−13 |
| 192 | LI:1189543.1:2000MAY01 | 592 | 744 | forward 1 | KH-domain | KH domain | 5.40E−13 |
| 193 | LG:455450.1:2000FEB18 | 1 | 426 | forward 1 | dehydrin | Dehydrins | 4.20E−41 |
| 194 | LG:1040978.1:2000FEB18 | 278 | 481 | forward 2 | pp-binding | Phosphopantetheine attachment site | 3.90E−14 |
| 195 | LG:446649.1:2000FEB18 | 80 | 316 | forward 2 | ACBP | Acyl CoA binding protein | 1.60E−44 |
| 196 | LG:132147.3:2000FEB18 | 1497 | 2414 | forward 3 | HECT | HECT-domain (ubiquitin-transferase). | 9.90E−138 |
| 196 | LG:132147.3:2000FEB18 | 1065 | 1154 | forward 3 | WW | WW domain | 1.50E−12 |
| 198 | LG:162161.1:2000MAY19 | 128 | 385 | forward 2 | TIG | IPT/TIG domain | 5.50E−15 |
| 200 | LG:204626.1:2000MAY19 | 322 | 1212 | forward 1 | Syntaxin | Syntaxin | 8.60E−44 |
| 202 | LI:476342.1:2000MAY01 | 159 | 299 | forward 3 | Gamma-thionin | Gamma-thionins family | 1.70E−19 |
| 205 | LG:482261.1:2000FEB18 | 286 | 552 | forward 1 | Gag_p10 | Retroviral GAG p10 protein | 6.80E−31 |
| 205 | LG:482261.1:2000FEB18 | 1044 | 1229 | forward 3 | gag_p24 | gag gene protein p24 (core nucleocapsid | 2.00E−15 |
| 205 | LG:482261.1:2000FEB18 | 1375 | 1545 | forward 1 | gag_p24 | gag gene protein p24 (core nucleocapsid | 9.50E−15 |
| 206 | LG:480328.1:2000FEB18 | 985 | 1515 | forward 1 | Band_7 | SPFH domain/Band 7 family | 2.60E−39 |
| 206 | LG:480328.1:2000FEB18 | 49 | 117 | forward 1 | zf-C2H2 | Zinc finger, C2H2 type | 1.60E−06 |
| 210 | LG:380497.2:2000MAY19 | 202 | 336 | forward 1 | G-patch | G-patch domain | 7.00E−17 |

TABLE 3

| | | | | | Domain | |
|---|---|---|---|---|---|---|
| SEQ ID NO: | Template ID | Start | Stop | Frame | Type | Topology |
| 1 | LG:1040582.1:2000FEB18 | 31 | 117 | forward 1 | TM | N in |
| 1 | LG:1040582.1:2000FEB18 | 319 | 405 | forward 1 | TM | N in |
| 1 | LG:1040582.1:2000FEB18 | 108 | 155 | forward 3 | TM | N out |
| 2 | LG:453570.1:2000FEB18 | 361 | 447 | forward 1 | TM | N in |
| 3 | LG:408751.3:2000FEB18 | 1318 | 1404 | forward 1 | TM | N in |
| 3 | LG:408751.3:2000FEB18 | 1025 | 1099 | forward 2 | TM | N in |
| 3 | LG:408751.3:2000FEB18 | 1298 | 1360 | forward 2 | TM | N in |
| 3 | LG:408751.3:2000FEB18 | 1379 | 1441 | forward 2 | TM | N in |
| 3 | LG:408751.3:2000FEB18 | 1463 | 1537 | forward 2 | TM | N in |
| 3 | LG:408751.3:2000FEB18 | 1047 | 1133 | forward 3 | TM | N in |
| 3 | LG:408751.3:2000FEB18 | 1266 | 1352 | forward 3 | TM | N in |
| 3 | LG:408751.3:2000FEB18 | 1419 | 1469 | forward 3 | TM | N in |
| 4 | LI:090574.1:2000FEB01 | 79 | 144 | forward 1 | TM | N in |
| 4 | LI:090574.1:2000FEB01 | 607 | 678 | forward 1 | TM | N in |
| 4 | LI:090574.1:2000FEB01 | 1009 | 1080 | forward 1 | TM | N in |
| 4 | LI:090574.1:2000FEB01 | 497 | 583 | forward 2 | TM | N out |
| 4 | LI:090574.1:2000FEB01 | 743 | 829 | forward 2 | TM | N out |
| 4 | LI:090574.1:2000FEB01 | 1026 | 1085 | forward 3 | TM | N out |
| 5 | LI:229932.2:2000FEB01 | 76 | 162 | forward 1 | TM | N out |
| 5 | LI:229932.2:2000FEB01 | 190 | 276 | forward 1 | TM | N out |
| 5 | LI:229932.2:2000FEB01 | 1237 | 1323 | forward 1 | TM | N out |
| 5 | LI:229932.2:2000FEB01 | 68 | 142 | forward 2 | TM | N in |
| 5 | LI:229932.2:2000FEB01 | 335 | 412 | forward 2 | TM | N in |
| 5 | LI:229932.2:2000FEB01 | 758 | 844 | forward 2 | TM | N in |
| 5 | LI:229932.2:2000FEB01 | 1229 | 1288 | forward 2 | TM | N in |
| 5 | LI:229932.2:2000FEB01 | 60 | 146 | forward 3 | TM | N in |
| 5 | LI:229932.2:2000FEB01 | 216 | 302 | forward 3 | TM | N in |
| 5 | LI:229932.2:2000FEB01 | 690 | 752 | forward 3 | TM | N in |
| 5 | LI:229932.2:2000FEB01 | 765 | 827 | forward 3 | TM | N in |
| 5 | LI:229932.2:2000FEB01 | 1209 | 1289 | forward 3 | TM | N in |
| 6 | LI:332176.1:2000FEB01 | 343 | 399 | forward 1 | TM | N in |
| 6 | LI:332176.1:2000FEB01 | 1078 | 1131 | forward 1 | TM | N in |
| 6 | LI:332176.1:2000FEB01 | 1606 | 1692 | forward 1 | TM | N in |
| 6 | LI:332176.1:2000FEB01 | 2218 | 2274 | forward 1 | TM | N in |
| 6 | LI:332176.1:2000FEB01 | 2383 | 2433 | forward 1 | TM | N in |
| 6 | LI:332176.1:2000FEB01 | 110 | 196 | forward 2 | TM | N in |
| 6 | LI:332176.1:2000FEB01 | 1307 | 1378 | forward 2 | TM | N in |
| 6 | LI:332176.1:2000FEB01 | 1640 | 1726 | forward 2 | TM | N in |
| 6 | LI:332176.1:2000FEB01 | 1946 | 2005 | forward 2 | TM | N in |
| 6 | LI:332176.1:2000FEB01 | 135 | 200 | forward 3 | TM | N in |
| 6 | LI:332176.1:2000FEB01 | 693 | 752 | forward 3 | TM | N in |
| 6 | LI:332176.1:2000FEB01 | 777 | 839 | forward 3 | TM | N in |
| 6 | LI:332176.1:2000FEB01 | 867 | 929 | forward 3 | TM | N in |
| 6 | LI:332176.1:2000FEB01 | 1035 | 1118 | forward 3 | TM | N in |
| 6 | LI:332176.1:2000FEB01 | 1173 | 1253 | forward 3 | TM | N in |
| 6 | LI:332176.1:2000FEB01 | 1572 | 1658 | forward 3 | TM | N in |
| 6 | LI:332176.1:2000FEB01 | 2121 | 2180 | forward 3 | TM | N in |
| 6 | LI:332176.1:2000FEB01 | 2277 | 2363 | forward 3 | TM | N in |
| 6 | LI:332176.1:2000FEB01 | 2400 | 2456 | forward 3 | TM | N in |
| 8 | LG:220992.1:2000MAY19 | 343 | 393 | forward 1 | TM | |
| 8 | LG:220992.1:2000MAY19 | 646 | 732 | forward 1 | TM | |
| 8 | LG:220992.1:2000MAY19 | 1639 | 1725 | forward 1 | TM | |
| 8 | LG:220992.1:2000MAY19 | 1879 | 1965 | forward 1 | TM | |
| 8 | LG:220992.1:2000MAY19 | 2005 | 2088 | forward 1 | TM | |
| 8 | LG:220992.1:2000MAY19 | 17 | 76 | forward 2 | TM | N in |
| 8 | LG:220992.1:2000MAY19 | 1646 | 1732 | forward 2 | TM | N in |
| 8 | LG:220992.1:2000MAY19 | 1850 | 1933 | forward 2 | TM | N in |
| 8 | LG:220992.1:2000MAY19 | 1434 | 1484 | forward 3 | TM | N out |
| 8 | LG:220992.1:2000MAY19 | 1734 | 1820 | forward 3 | TM | N out |
| 8 | LG:220992.1:2000MAY19 | 1974 | 2036 | forward 3 | TM | N out |
| 8 | LG:220992.1:2000MAY19 | 2067 | 2129 | forward 3 | TM | N out |
| 8 | LG:220992.1:2000MAY19 | 2151 | 2237 | forward 3 | TM | N out |
| 9 | LG:1094571.1:2000MAY19 | 781 | 867 | forward 1 | TM | N in |
| 9 | LG:1094571.1:2000MAY19 | 419 | 505 | forward 2 | TM | N in |
| 9 | LG:1094571.1:2000MAY19 | 767 | 853 | forward 2 | TM | N in |
| 9 | LG:1094571.1:2000MAY19 | 756 | 842 | forward 3 | TM | N in |
| 10 | LI:350754.4:2000MAY01 | 277 | 348 | forward 1 | TM | N in |
| 10 | LI:350754.4:2000MAY01 | 583 | 651 | forward 1 | TM | N in |
| 10 | LI:350754.4:2000MAY01 | 670 | 747 | forward 1 | TM | N in |
| 10 | LI:350754.4:2000MAY01 | 381 | 467 | forward 3 | TM | N in |
| 10 | LI:350754.4:2000MAY01 | 2469 | 2555 | forward 3 | TM | N in |
| 12 | LI:1190263.1:2000MAY01 | 664 | 735 | forward 1 | TM | N in |
| 12 | LI:1190263.1:2000MAY01 | 787 | 861 | forward 1 | TM | N in |
| 12 | LI:1190263.1:2000MAY01 | 901 | 954 | forward 1 | TM | N in |
| 12 | LI:1190263.1:2000MAY01 | 188 | 274 | forward 2 | TM | N in |
| 12 | LI:1190263.1:2000MAY01 | 455 | 508 | forward 2 | TM | N in |
| 12 | LI:1190263.1:2000MAY01 | 809 | 895 | forward 2 | TM | N in |
| 12 | LI:1190263.1:2000MAY01 | 1616 | 1663 | forward 2 | TM | N in |
| 12 | LI:1190263.1:2000MAY01 | 183 | 251 | forward 3 | TM | N in |
| 12 | LI:1190263.1:2000MAY01 | 648 | 704 | forward 3 | TM | N in |
| 12 | LI:1190263.1:2000MAY01 | 1149 | 1235 | forward 3 | TM | N in |
| 13 | LG:270916.2:2000FEB18 | 173 | 259 | forward 2 | TM | N out |
| 14 | LG:999414.3:2000FEB18 | 109 | 195 | forward 1 | TM | N out |
| 14 | LG:999414.3:2000FEB18 | 358 | 438 | forward 1 | TM | N out |
| 14 | LG:999414.3:2000FEB18 | 520 | 591 | forward 1 | TM | N out |
| 14 | LG:999414.3:2000FEB18 | 661 | 744 | forward 1 | TM | N out |
| 14 | LG:999414.3:2000FEB18 | 883 | 969 | forward 1 | TM | N out |
| 14 | LG:999414.3:2000FEB18 | 976 | 1062 | forward 1 | TM | N out |
| 14 | LG:999414.3:2000FEB18 | 302 | 388 | forward 2 | TM | N in |
| 14 | LG:999414.3:2000FEB18 | 533 | 613 | forward 2 | TM | N in |
| 14 | LG:999414.3:2000FEB18 | 992 | 1048 | forward 2 | TM | N in |
| 14 | LG:999414.3:2000FEB18 | 1169 | 1246 | forward 2 | TM | N in |
| 14 | LG:999414.3:2000FEB18 | 1307 | 1366 | forward 2 | TM | N in |
| 14 | LG:999414.3:2000FEB18 | 207 | 284 | forward 3 | TM | N out |
| 14 | LG:999414.3:2000FEB18 | 324 | 404 | forward 3 | TM | N out |
| 14 | LG:999414.3:2000FEB18 | 540 | 599 | forward 3 | TM | N out |
| 14 | LG:999414.3:2000FEB18 | 1029 | 1115 | forward 3 | TM | N out |
| 14 | LG:999414.3:2000FEB18 | 1167 | 1253 | forward 3 | TM | N out |
| 14 | LG:999414.3:2000FEB18 | 1314 | 1373 | forward 3 | TM | N out |
| 15 | LG:429446.1:2000FEB18 | 628 | 699 | forward 1 | TM | N out |
| 15 | LG:429446.1:2000FEB18 | 629 | 682 | forward 2 | TM | N in |
| 15 | LG:429446.1:2000FEB18 | 627 | 713 | forward 3 | TM | N in |
| 16 | LI:057229.1:2000FEB01 | 10 | 69 | forward 1 | TM | |
| 16 | LI:057229.1:2000FEB01 | 118 | 198 | forward 1 | TM | |
| 16 | LI:057229.1:2000FEB01 | 292 | 360 | forward 1 | TM | |
| 16 | LI:057229.1:2000FEB01 | 11 | 67 | forward 2 | TM | |
| 16 | LI:057229.1:2000FEB01 | 146 | 226 | forward 2 | TM | |
| 16 | LI:057229.1:2000FEB01 | 290 | 355 | forward 2 | TM | |
| 16 | LI:057229.1:2000FEB01 | 12 | 71 | forward 3 | TM | N out |
| 16 | LI:057229.1:2000FEB01 | 114 | 176 | forward 3 | TM | N out |
| 17 | LI:351965.1:2000FEB01 | 487 | 573 | forward 1 | TM | |
| 17 | LI:351965.1:2000FEB01 | 1036 | 1098 | forward 1 | TM | |
| 17 | LI:351965.1:2000FEB01 | 492 | 578 | forward 3 | TM | N in |
| 17 | LI:351965.1:2000FEB01 | 969 | 1055 | forward 3 | TM | N in |
| 17 | LI:351965.1:2000FEB01 | 1098 | 1184 | forward 3 | TM | N in |
| 18 | LG:068682.1:2000FEB18 | 707 | 793 | forward 2 | TM | N out |
| 19 | LG:242665.1:2000FEB18 | 10 | 63 | forward 1 | TM | N out |
| 19 | LG:242665.1:2000FEB18 | 12 | 62 | forward 3 | TM | N out |
| 19 | LG:242665.1:2000FEB18 | 333 | 398 | forward 3 | TM | N out |
| 20 | LG:241743.1:2000FEB18 | 43 | 99 | forward 1 | TM | N out |
| 21 | LI:034212.1:2000FEB01 | 1300 | 1365 | forward 1 | TM | N in |
| 21 | LI:034212.1:2000FEB01 | 1570 | 1647 | forward 1 | TM | N in |
| 21 | LI:034212.1:2000FEB01 | 2386 | 2472 | forward 1 | TM | N in |
| 21 | LI:034212.1:2000FEB01 | 2533 | 2598 | forward 1 | TM | N in |
| 21 | LI:034212.1:2000FEB01 | 2620 | 2706 | forward 1 | TM | N in |
| 21 | LI:034212.1:2000FEB01 | 2740 | 2826 | forward 1 | TM | N in |
| 21 | LI:034212.1:2000FEB01 | 719 | 805 | forward 2 | TM | |
| 21 | LI:034212.1:2000FEB01 | 1205 | 1291 | forward 2 | TM | |
| 21 | LI:034212.1:2000FEB01 | 1460 | 1546 | forward 2 | TM | |
| 21 | LI:034212.1:2000FEB01 | 1685 | 1768 | forward 2 | TM | |
| 21 | LI:034212.1:2000FEB01 | 1814 | 1882 | forward 2 | TM | |
| 21 | LI:034212.1:2000FEB01 | 2066 | 2128 | forward 2 | TM | |
| 21 | LI:034212.1:2000FEB01 | 2156 | 2218 | forward 2 | TM | |
| 21 | LI:034212.1:2000FEB01 | 2540 | 2626 | forward 2 | TM | |
| 21 | LI:034212.1:2000FEB01 | 2657 | 2734 | forward 2 | TM | |
| 21 | LI:034212.1:2000FEB01 | 12 | 62 | forward 3 | TM | N out |
| 21 | LI:034212.1:2000FEB01 | 1236 | 1301 | forward 3 | TM | N out |
| 21 | LI:034212.1:2000FEB01 | 1590 | 1646 | forward 3 | TM | N out |
| 21 | LI:034212.1:2000FEB01 | 1668 | 1721 | forward 3 | TM | N out |
| 21 | LI:034212.1:2000FEB01 | 2130 | 2216 | forward 3 | TM | N out |
| 21 | LI:034212.1:2000FEB01 | 2295 | 2381 | forward 3 | TM | N out |
| 21 | LI:034212.1:2000FEB01 | 2436 | 2513 | forward 3 | TM | N out |
| 21 | LI:034212.1:2000FEB01 | 2538 | 2624 | forward 3 | TM | N out |
| 21 | LI:034212.1:2000FEB01 | 2667 | 2735 | forward 3 | TM | N out |
| 22 | LG:344886.1:2000MAY19 | 937 | 1002 | forward 1 | TM | N in |
| 22 | LG:344886.1:2000MAY19 | 1081 | 1155 | forward 1 | TM | N in |
| 22 | LG:344886.1:2000MAY19 | 1696 | 1782 | forward 1 | TM | N in |
| 22 | LG:344886.1:2000MAY19 | 413 | 463 | forward 2 | TM | N in |
| 22 | LG:344886.1:2000MAY19 | 551 | 637 | forward 2 | TM | N in |
| 22 | LG:344886.1:2000MAY19 | 950 | 1012 | forward 2 | TM | N in |
| 22 | LG:344886.1:2000MAY19 | 1031 | 1093 | forward 2 | TM | N in |
| 22 | LG:344886.1:2000MAY19 | 1112 | 1183 | forward 2 | TM | N in |
| 22 | LG:344886.1:2000MAY19 | 1271 | 1348 | forward 2 | TM | N in |
| 22 | LG:344886.1:2000MAY19 | 1634 | 1720 | forward 2 | TM | N in |
| 22 | LG:344886.1:2000MAY19 | 567 | 626 | forward 3 | TM | N in |
| 22 | LG:344886.1:2000MAY19 | 1011 | 1073 | forward 3 | TM | N in |
| 22 | LG:344886.1:2000MAY19 | 1089 | 1151 | forward 3 | TM | N in |
| 22 | LG:344886.1:2000MAY19 | 1707 | 1757 | forward 3 | TM | N in |
| 23 | LG:228930.1:2000MAY19 | 111 | 167 | forward 3 | TM | N in |
| 24 | LG:338927.1:2000MAY19 | 934 | 1020 | forward 1 | TM | N out |
| 24 | LG:338927.1:2000MAY19 | 1133 | 1219 | forward 2 | TM | N in |
| 24 | LG:338927.1:2000MAY19 | 1170 | 1250 | forward 3 | TM | N in |
| 25 | LG:898771.1:2000MAY19 | 1261 | 1314 | forward 1 | TM | N out |
| 25 | LG:898771.1:2000MAY19 | 1397 | 1450 | forward 2 | TM | N out |
| 26 | LI:257664.67:2000MAY01 | 280 | 366 | forward 1 | TM | N in |
| 26 | LI:257664.67:2000MAY01 | 421 | 498 | forward 1 | TM | N in |
| 26 | LI:257664.67:2000MAY01 | 12 | 71 | forward 3 | TM | N out |
| 27 | LI:001496.2:2000MAY01 | 399 | 473 | forward 3 | TM | |
| 28 | LI:1085273.2:2000MAY01 | 2188 | 2274 | forward 1 | TM | N in |
| 28 | LI:1085273.2:2000MAY01 | 503 | 583 | forward 2 | TM | N out |
| 28 | LI:1085273.2:2000MAY01 | 2126 | 2194 | forward 2 | TM | N out |
| 28 | LI:1085273.2:2000MAY01 | 897 | 968 | forward 3 | TM | N in |
| 29 | LI:333138.2:2000MAY01 | 1930 | 2016 | forward 1 | TM | N out |
| 29 | LI:333138.2:2000MAY01 | 50 | 103 | forward 2 | TM | |
| 29 | LI:333138.2:2000MAY01 | 884 | 940 | forward 2 | TM | |
| 29 | LI:333138.2:2000MAY01 | 114 | 179 | forward 3 | TM | N out |
| 29 | LI:333138.2:2000MAY01 | 273 | 356 | forward 3 | TM | N out |
| 29 | LI:333138.2:2000MAY01 | 819 | 875 | forward 3 | TM | N out |
| 29 | LI:333138.2:2000MAY01 | 1581 | 1667 | forward 3 | TM | N out |
| 30 | LI:338927.1:2000MAY01 | 1069 | 1140 | forward 1 | TM | N in |
| 30 | LI:338927.1:2000MAY01 | 968 | 1051 | forward 2 | TM | N in |
| 30 | LI:338927.1:2000MAY01 | 1056 | 1118 | forward 3 | TM | N out |
| 30 | LI:338927.1:2000MAY01 | 1155 | 1217 | forward 3 | TM | N out |
| 31 | LG:335558.1:2000FEB18 | 518 | 604 | forward 2 | TM | N in |
| 31 | LG:335558.1:2000FEB18 | 614 | 682 | forward 2 | TM | N in |
| 31 | LG:335558.1:2000FEB18 | 761 | 829 | forward 2 | TM | N in |
| 31 | LG:335558.1:2000FEB18 | 798 | 860 | forward 3 | TM | N in |
| 31 | LG:335558.1:2000FEB18 | 882 | 944 | forward 3 | TM | N in |
| 31 | LG:335558.1:2000FEB18 | 966 | 1028 | forward 3 | TM | N in |
| 32 | LG:998283.7:2000FEB18 | 1066 | 1146 | forward 1 | TM | N in |
| 32 | LG:998283.7:2000FEB18 | 23 | 109 | forward 2 | TM | N in |
| 32 | LG:998283.7:2000FEB18 | 194 | 280 | forward 2 | TM | N in |
| 32 | LG:998283.7:2000FEB18 | 392 | 478 | forward 2 | TM | N in |
| 32 | LG:998283.7:2000FEB18 | 527 | 613 | forward 2 | TM | N in |
| 32 | LG:998283.7:2000FEB18 | 776 | 862 | forward 2 | TM | N in |
| 32 | LG:998283.7:2000FEB18 | 1064 | 1141 | forward 2 | TM | N in |
| 32 | LG:998283.7:2000FEB18 | 12 | 65 | forward 3 | TM | N in |
| 32 | LG:998283.7:2000FEB18 | 147 | 227 | forward 3 | TM | N in |
| 32 | LG:998283.7:2000FEB18 | 684 | 770 | forward 3 | TM | N in |
| 32 | LG:998283.7:2000FEB18 | 1011 | 1097 | forward 3 | TM | N in |
| 33 | LI:402739.1:2000FEB01 | 415 | 501 | forward 1 | TM | N in |
| 35 | LG:981076.2:2000MAY19 | 388 | 450 | forward 1 | TM | N in |
| 35 | LG:981076.2:2000MAY19 | 20 | 82 | forward 2 | TM | N out |
| 35 | LG:981076.2:2000MAY19 | 389 | 451 | forward 2 | TM | N out |
| 35 | LG:981076.2:2000MAY19 | 464 | 526 | forward 2 | TM | N out |
| 35 | LG:981076.2:2000MAY19 | 539 | 604 | forward 2 | TM | N out |
| 35 | LG:981076.2:2000MAY19 | 438 | 524 | forward 3 | TM | N in |
| 37 | LI:1190250.1:2000MAY01 | 530 | 613 | forward 2 | TM | |
| 37 | LI:1190250.1:2000MAY01 | 558 | 635 | forward 3 | TM | N out |
| 38 | LG:021371.3:2000FEB18 | 122 | 208 | forward 2 | TM | N in |
| 41 | LG:410726.1:2000FEB18 | 22 | 108 | forward 1 | TM | N in |
| 41 | LG:410726.1:2000FEB18 | 385 | 471 | forward 1 | TM | N in |
| 42 | LG:200005.1:2000FEB18 | 166 | 222 | forward 1 | TM | N out |
| 42 | LG:200005.1:2000FEB18 | 185 | 232 | forward 2 | TM | N out |
| 42 | LG:200005.1:2000FEB18 | 162 | 248 | forward 3 | TM | N out |
| 46 | LG:1079203.1:2000FEB18 | 11 | 70 | forward 2 | TM | N in |
| 46 | LG:1079203.1:2000FEB18 | 125 | 196 | forward 2 | TM | N in |
| 46 | LG:1079203.1:2000FEB18 | 965 | 1051 | forward 2 | TM | N in |
| 47 | LG:1082586.1:2000FEB18 | 256 | 339 | forward 1 | TM | N in |
| 47 | LG:1082586.1:2000FEB18 | 248 | 316 | forward 2 | TM | N out |
| 49 | LG:1082775.1:2000FEB18 | 553 | 606 | forward 1 | TM | N in |
| 50 | LG:1083120.1:2000FEB18 | 214 | 291 | forward 1 | TM | N out |
| 50 | LG:1083120.1:2000FEB18 | 233 | 319 | forward 2 | TM | N out |
| 50 | LG:1083120.1:2000FEB18 | 252 | 320 | forward 3 | TM | N in |
| 51 | LG:1087707.1:2000FEB18 | 367 | 453 | forward 1 | TM | N out |
| 51 | LG:1087707.1:2000FEB18 | 469 | 531 | forward 1 | TM | N out |
| 51 | LG:1087707.1:2000FEB18 | 667 | 729 | forward 1 | TM | N out |
| 51 | LG:1087707.1:2000FEB18 | 742 | 804 | forward 1 | TM | N out |
| 51 | LG:1087707.1:2000FEB18 | 407 | 481 | forward 2 | TM | N in |
| 51 | LG:1087707.1:2000FEB18 | 671 | 739 | forward 2 | TM | N in |
| 51 | LG:1087707.1:2000FEB18 | 743 | 811 | forward 2 | TM | N in |
| 51 | LG:1087707.1:2000FEB18 | 570 | 641 | forward 3 | TM | N out |
| 51 | LG:1087707.1:2000FEB18 | 747 | 833 | forward 3 | TM | N out |
| 52 | LG:1090915.1:2000FEB18 | 11 | 61 | forward 2 | TM | N out |
| 53 | LG:1094230.1:2000FEB18 | 469 | 555 | forward 1 | TM | N out |
| 53 | LG:1094230.1:2000FEB18 | 449 | 535 | forward 2 | TM | N out |
| 54 | LG:474848.3:2000FEB18 | 445 | 531 | forward 1 | TM | N out |
| 54 | LG:474848.3:2000FEB18 | 456 | 518 | forward 3 | TM | N out |
| 58 | LI:236654.2:2000FEB01 | 221 | 307 | forward 2 | TM | N out |
| 59 | LI:200009.1:2000FEB01 | 1045 | 1131 | forward 1 | TM | N out |
| 59 | LI:200009.1:2000FEB01 | 1171 | 1233 | forward 1 | TM | N out |
| 59 | LI:200009.1:2000FEB01 | 1076 | 1162 | forward 2 | TM | N in |
| 59 | LI:200009.1:2000FEB01 | 1044 | 1130 | forward 3 | TM | N in |
| 60 | LI:758502.1:2000FEB01 | 286 | 369 | forward 1 | TM | N out |
| 60 | LI:758502.1:2000FEB01 | 755 | 805 | forward 2 | TM | N in |
| 60 | LI:758502.1:2000FEB01 | 780 | 833 | forward 3 | TM | N in |
| 62 | LI:789445.1:2000FEB01 | 9 | 80 | forward 3 | TM | N out |
| 63 | LI:789657.1:2000FEB01 | 854 | 937 | forward 2 | TM | N in |
| 64 | LI:789808.1:2000FEB01 | 347 | 400 | forward 2 | TM | N in |
| 65 | LI:792919.1:2000FEB01 | 176 | 256 | forward 2 | TM | |
| 65 | LI:792919.1:2000FEB01 | 371 | 427 | forward 2 | TM | |
| 66 | LI:793949.1:2000FEB01 | 208 | 282 | forward 1 | TM | N out |
| 66 | LI:793949.1:2000FEB01 | 472 | 558 | forward 1 | TM | N out |
| 66 | LI:793949.1:2000FEB01 | 455 | 541 | forward 2 | TM | N out |
| 67 | LI:794389.1:2000FEB01 | 265 | 333 | forward 1 | TM | N out |
| 67 | LI:794389.1:2000FEB01 | 424 | 477 | forward 1 | TM | N out |
| 67 | LI:794389.1:2000FEB01 | 384 | 455 | forward 3 | TM | N in |
| 68 | LI:796010.1:2000FEB01 | 351 | 404 | forward 3 | TM | N in |
| 69 | LI:796324.1:2000FEB01 | 365 | 418 | forward 2 | TM | N in |
| 72 | LI:798636.1:2000FEB01 | 490 | 543 | forward 1 | TM | N in |
| 73 | LI:800045.1:2000FEB01 | 627 | 701 | forward 3 | TM | N in |
| 74 | LI:800680.1:2000FEB01 | 334 | 411 | forward 1 | TM | N out |
| 74 | LI:800680.1:2000FEB01 | 359 | 421 | forward 2 | TM | N out |
| 75 | LI:800894.1:2000FEB01 | 536 | 592 | forward 2 | TM | N in |
| 75 | LI:800894.1:2000FEB01 | 300 | 374 | forward 3 | TM | N out |
| 75 | LI:800894.1:2000FEB01 | 396 | 482 | forward 3 | TM | N out |
| 77 | LI:801236.1:2000FEB01 | 262 | 318 | forward 1 | TM | N out |
| 78 | LI:803335.1:2000FEB01 | 412 | 498 | forward 1 | TM | N out |
| 78 | LI:803335.1:2000FEB01 | 423 | 485 | forward 3 | TM | N out |
| 79 | LI:803998.1:2000FEB01 | 221 | 307 | forward 2 | TM | N out |
| 81 | LI:808532.1:2000FEB01 | 472 | 558 | forward 1 | TM | N in |
| 81 | LI:808532.1:2000FEB01 | 117 | 203 | forward 3 | TM | N in |
| 81 | LI:808532.1:2000FEB01 | 363 | 443 | forward 3 | TM | N in |
| 81 | LI:808532.1:2000FEB01 | 558 | 623 | forward 3 | TM | N in |
| 82 | LI:443073.1:2000FEB01 | 293 | 379 | forward 2 | TM | N in |
| 82 | LI:443073.1:2000FEB01 | 81 | 152 | forward 3 | TM | N in |
| 82 | LI:443073.1:2000FEB01 | 189 | 260 | forward 3 | TM | N in |
| 83 | LI:479671.1:2000FEB01 | 523 | 579 | forward 1 | TM | N out |
| 85 | LI:810224.1:2000FEB01 | 246 | 299 | forward 3 | TM | |
| 87 | LG:892274.1:2000MAY19 | 49 | 105 | forward 1 | TM | N out |
| 87 | LG:892274.1:2000MAY19 | 613 | 681 | forward 1 | TM | N out |
| 87 | LG:892274.1:2000MAY19 | 506 | 589 | forward 2 | TM | N in |
| 91 | LG:1084051.1:2000MAY19 | 301 | 363 | forward 1 | TM | N in |
| 92 | LG:1076853.1:2000MAY19 | 964 | 1050 | forward 1 | TM | N in |
| 92 | LG:1076853.1:2000MAY19 | 56 | 130 | forward 2 | TM | N out |
| 92 | LG:1076853.1:2000MAY19 | 741 | 818 | forward 3 | TM | N in |
| 93 | LG:481631.10:2000MAY19 | 298 | 357 | forward 1 | TM | N out |
| 93 | LG:481631.10:2000MAY19 | 598 | 654 | forward 1 | TM | N out |
| 94 | LG:1088431.2:2000MAY19 | 379 | 441 | forward 1 | TM | N out |
| 94 | LG:1088431.2:2000MAY19 | 354 | 431 | forward 3 | TM | N out |
| 95 | LI:401619.10:2000MAY01 | 157 | 219 | forward 1 | TM | N out |
| 95 | LI:401619.10:2000MAY01 | 232 | 294 | forward 1 | TM | N out |
| 95 | LI:401619.10:2000MAY01 | 502 | 576 | forward 1 | TM | N out |
| 95 | LI:401619.10:2000MAY01 | 146 | 232 | forward 2 | TM | N in |
| 95 | LI:401619.10:2000MAY01 | 326 | 412 | forward 2 | TM | N in |
| 95 | LI:401619.10:2000MAY01 | 440 | 490 | forward 2 | TM | N in |
| 95 | LI:401619.10:2000MAY01 | 512 | 580 | forward 2 | TM | N in |
| 95 | LI:401619.10:2000MAY01 | 186 | 257 | forward 3 | TM | N in |
| 95 | LI:401619.10:2000MAY01 | 528 | 599 | forward 3 | TM | N in |
| 96 | LI:1144007.1:2000MAY01 | 2833 | 2910 | forward 1 | TM | N in |
| 96 | LI:1144007.1:2000MAY01 | 3301 | 3378 | forward 1 | TM | N in |
| 96 | LI:1144007.1:2000MAY01 | 3511 | 3597 | forward 1 | TM | N in |
| 96 | LI:1144007.1:2000MAY01 | 3634 | 3696 | forward 1 | TM | N in |
| 96 | LI:1144007.1:2000MAY01 | 3736 | 3801 | forward 1 | TM | N in |
| 96 | LI:1144007.1:2000MAY01 | 2645 | 2725 | forward 2 | TM | N out |
| 96 | LI:1144007.1:2000MAY01 | 2879 | 2965 | forward 2 | TM | N out |
| 96 | LI:1144007.1:2000MAY01 | 3356 | 3433 | forward 2 | TM | N out |
| 96 | LI:1144007.1:2000MAY01 | 3476 | 3523 | forward 2 | TM | N out |
| 96 | LI:1144007.1:2000MAY01 | 2772 | 2858 | forward 3 | TM | N in |
| 96 | LI:1144007.1:2000MAY01 | 3258 | 3332 | forward 3 | TM | N in |
| 96 | LI:1144007.1:2000MAY01 | 4017 | 4097 | forward 3 | TM | N in |
| 97 | LI:331074.1:2000MAY01 | 1264 | 1326 | forward 1 | TM | N in |
| 97 | LI:331074.1:2000MAY01 | 1357 | 1419 | forward 1 | TM | N in |
| 97 | LI:331074.1:2000MAY01 | 1450 | 1512 | forward 1 | TM | N in |
| 97 | LI:331074.1:2000MAY01 | 1540 | 1626 | forward 1 | TM | N in |
| 97 | LI:331074.1:2000MAY01 | 1433 | 1513 | forward 2 | TM | N in |
| 97 | LI:331074.1:2000MAY01 | 1574 | 1660 | forward 2 | TM | N in |
| 97 | LI:331074.1:2000MAY01 | 1461 | 1529 | forward 3 | TM | N in |
| 97 | LI:331074.1:2000MAY01 | 1560 | 1646 | forward 3 | TM | N in |
| 98 | LI:1170349.1:2000MAY01 | 34 | 102 | forward 1 | TM | N in |
| 99 | LG:335097.1:2000FEB18 | 601 | 672 | forward 1 | TM | N out |
| 99 | LG:335097.1:2000FEB18 | 847 | 909 | forward 1 | TM | N out |
| 99 | LG:335097.1:2000FEB18 | 928 | 981 | forward 1 | TM | N out |
| 99 | LG:335097.1:2000FEB18 | 164 | 244 | forward 2 | TM | N out |
| 99 | LG:335097.1:2000FEB18 | 623 | 682 | forward 2 | TM | N out |
| 99 | LG:335097.1:2000FEB18 | 12 | 74 | forward 3 | TM | N in |
| 99 | LG:335097.1:2000FEB18 | 219 | 299 | forward 3 | TM | N in |
| 99 | LG:335097.1:2000FEB18 | 594 | 680 | forward 3 | TM | N in |
| 100 | LG:1076451.1:2000FEB18 | 94 | 156 | forward 1 | TM | N in |
| 100 | LG:1076451.1:2000FEB18 | 101 | 187 | forward 2 | TM | N out |
| 100 | LG:1076451.1:2000FEB18 | 18 | 98 | forward 3 | TM | N out |
| 100 | LG:1076451.1:2000FEB18 | 96 | 164 | forward 3 | TM | N out |
| 100 | LG:1076451.1:2000FEB18 | 216 | 290 | forward 3 | TM | N out |
| 101 | LI:805478.1:2000FEB01 | 83 | 136 | forward 2 | TM | N out |
| 101 | LI:805478.1:2000FEB01 | 212 | 298 | forward 2 | TM | N out |
| 102 | LG:101269.1:2000MAY19 | 655 | 741 | forward 1 | TM | N in |
| 102 | LG:101269.1:2000MAY19 | 650 | 736 | forward 2 | TM | N in |
| 102 | LG:101269.1:2000MAY19 | 96 | 182 | forward 3 | TM | N in |
| 102 | LG:101269.1:2000MAY19 | 249 | 335 | forward 3 | TM | N in |
| 102 | LG:101269.1:2000MAY19 | 663 | 740 | forward 3 | TM | N in |
| 103 | LI:331087.1:2000MAY01 | 251 | 298 | forward 2 | TM | N out |
| 103 | LI:331087.1:2000MAY01 | 237 | 311 | forward 3 | TM | |
| 104 | LI:410188.1:2000MAY01 | 520 | 591 | forward 1 | TM | N in |
| 104 | LI:410188.1:2000MAY01 | 640 | 711 | forward 1 | TM | N in |
| 104 | LI:410188.1:2000MAY01 | 724 | 810 | forward 1 | TM | N in |
| 104 | LI:410188.1:2000MAY01 | 832 | 879 | forward 1 | TM | N in |
| 104 | LI:410188.1:2000MAY01 | 883 | 969 | forward 1 | TM | N in |
| 104 | LI:410188.1:2000MAY01 | 1171 | 1257 | forward 1 | TM | N in |
| 104 | LI:410188.1:2000MAY01 | 1303 | 1389 | forward 1 | TM | N in |
| 104 | LI:410188.1:2000MAY01 | 2290 | 2361 | forward 1 | TM | N in |
| 104 | LI:410188.1:2000MAY01 | 2389 | 2460 | forward 1 | TM | N in |
| 104 | LI:410188.1:2000MAY01 | 2470 | 2556 | forward 1 | TM | N in |
| 104 | LI:410188.1:2000MAY01 | 2635 | 2721 | forward 1 | TM | N in |
| 104 | LI:410188.1:2000MAY01 | 2794 | 2862 | forward 1 | TM | N in |
| 104 | LI:410188.1:2000MAY01 | 2878 | 2964 | forward 1 | TM | N in |
| 104 | LI:410188.1:2000MAY01 | 3757 | 3837 | forward 1 | TM | N in |
| 104 | LI:410188.1:2000MAY01 | 3871 | 3957 | forward 1 | TM | N in |
| 104 | LI:410188.1:2000MAY01 | 3961 | 4047 | forward 1 | TM | N in |
| 104 | LI:410188.1:2000MAY01 | 4111 | 4194 | forward 1 | TM | N in |
| 104 | LI:410188.1:2000MAY01 | 4342 | 4428 | forward 1 | TM | N in |
| 104 | LI:410188.1:2000MAY01 | 4492 | 4578 | forward 1 | TM | N in |
| 104 | LI:410188.1:2000MAY01 | 4714 | 4794 | forward 1 | TM | N in |
| 104 | LI:410188.1:2000MAY01 | 6439 | 6519 | forward 1 | TM | N in |
| 104 | LI:410188.1:2000MAY01 | 7492 | 7575 | forward 1 | TM | N in |
| 104 | LI:410188.1:2000MAY01 | 7783 | 7845 | forward 1 | TM | N in |
| 104 | LI:410188.1:2000MAY01 | 4673 | 4735 | forward 2 | TM | N in |
| 104 | LI:410188.1:2000MAY01 | 4766 | 4828 | forward 2 | TM | N in |
| 104 | LI:410188.1:2000MAY01 | 4928 | 5014 | forward 2 | TM | N in |
| 104 | LI:410188.1:2000MAY01 | 5231 | 5317 | forward 2 | TM | N in |
| 104 | LI:410188.1:2000MAY01 | 6341 | 6409 | forward 2 | TM | N in |
| 104 | LI:410188.1:2000MAY01 | 7655 | 7741 | forward 2 | TM | N in |
| 104 | LI:410188.1:2000MAY01 | 8060 | 8146 | forward 2 | TM | N in |
| 104 | LI:410188.1:2000MAY01 | 4776 | 4859 | forward 3 | TM | N in |
| 104 | LI:410188.1:2000MAY01 | 6309 | 6371 | forward 3 | TM | N in |
| 104 | LI:410188.1:2000MAY01 | 7704 | 7775 | forward 3 | TM | N in |
| 105 | LI:1188288.1:2000MAY01 | 457 | 519 | forward 1 | TM | |
| 105 | LI:1188288.1:2000MAY01 | 841 | 915 | forward 1 | TM | |
| 105 | LI:1188288.1:2000MAY01 | 958 | 1038 | forward 1 | TM | |
| 105 | LI:1188288.1:2000MAY01 | 1072 | 1140 | forward 1 | TM | |
| 105 | LI:1188288.1:2000MAY01 | 1477 | 1539 | forward 1 | TM | |
| 105 | LI:1188288.1:2000MAY01 | 1564 | 1626 | forward 1 | TM | |
| 105 | LI:1188288.1:2000MAY01 | 1810 | 1896 | forward 1 | TM | |
| 105 | LI:1188288.1:2000MAY01 | 2134 | 2220 | forward 1 | TM | |
| 105 | LI:1188288.1:2000MAY01 | 2734 | 2820 | forward 1 | TM | |
| 105 | LI:1188288.1:2000MAY01 | 1067 | 1147 | forward 2 | TM | N out |
| 105 | LI:1188288.1:2000MAY01 | 1157 | 1243 | forward 2 | TM | N out |
| 105 | LI:1188288.1:2000MAY01 | 1313 | 1399 | forward 2 | TM | N out |
| 105 | LI:1188288.1:2000MAY01 | 1556 | 1618 | forward 2 | TM | N out |
| 105 | LI:1188288.1:2000MAY01 | 2294 | 2368 | forward 2 | TM | N out |
| 105 | LI:1188288.1:2000MAY01 | 435 | 521 | forward 3 | TM | N in |
| 105 | LI:1188288.1:2000MAY01 | 597 | 683 | forward 3 | TM | N in |
| 105 | LI:1188288.1:2000MAY01 | 2301 | 2354 | forward 3 | TM | N in |
| 105 | LI:1188288.1:2000MAY01 | 2700 | 2753 | forward 3 | TM | N in |
| 106 | LI:427997.4:2000MAY01 | 148 | 222 | forward 1 | TM | N in |
| 106 | LI:427997.4:2000MAY01 | 745 | 828 | forward 1 | TM | N in |
| 106 | LI:427997.4:2000MAY01 | 1192 | 1278 | forward 1 | TM | N in |
| 106 | LI:427997.4:2000MAY01 | 1351 | 1434 | forward 1 | TM | N in |
| 106 | LI:427997.4:2000MAY01 | 1450 | 1518 | forward 1 | TM | N in |
| 106 | LI:427997.4:2000MAY01 | 1759 | 1845 | forward 1 | TM | N in |
| 106 | LI:427997.4:2000MAY01 | 134 | 220 | forward 2 | TM | N in |
| 106 | LI:427997.4:2000MAY01 | 749 | 832 | forward 2 | TM | N in |
| 106 | LI:427997.4:2000MAY01 | 1031 | 1087 | forward 2 | TM | N in |
| 106 | LI:427997.4:2000MAY01 | 1607 | 1693 | forward 2 | TM | N in |
| 106 | LI:427997.4:2000MAY01 | 1730 | 1816 | forward 2 | TM | N in |
| 106 | LI:427997.4:2000MAY01 | 2111 | 2191 | forward 2 | TM | N in |
| 106 | LI:427997.4:2000MAY01 | 150 | 236 | forward 3 | TM | N in |
| 106 | LI:427997.4:2000MAY01 | 681 | 767 | forward 3 | TM | N in |
| 106 | LI:427997.4:2000MAY01 | 765 | 851 | forward 3 | TM | N in |
| 106 | LI:427997.4:2000MAY01 | 1068 | 1124 | forward 3 | TM | N in |
| 106 | LI:427997.4:2000MAY01 | 1665 | 1751 | forward 3 | TM | N in |
| 106 | LI:427997.4:2000MAY01 | 1782 | 1856 | forward 3 | TM | N in |
| 107 | LG:451682.1:2000FEB18 | 93 | 155 | forward 3 | TM | |
| 109 | LG:481436.5:2000FEB18 | 583 | 669 | forward 1 | TM | N in |
| 109 | LG:481436.5:2000FEB18 | 769 | 834 | forward 1 | TM | N in |
| 109 | LG:481436.5:2000FEB18 | 1111 | 1176 | forward 1 | TM | N in |
| 109 | LG:481436.5:2000FEB18 | 575 | 655 | forward 2 | TM | N out |
| 109 | LG:481436.5:2000FEB18 | 764 | 826 | forward 2 | TM | N out |
| 109 | LG:481436.5:2000FEB18 | 1091 | 1153 | forward 2 | TM | N out |
| 109 | LG:481436.5:2000FEB18 | 1187 | 1249 | forward 2 | TM | N out |
| 109 | LG:481436.5:2000FEB18 | 84 | 170 | forward 3 | TM | N in |
| 109 | LG:481436.5:2000FEB18 | 753 | 833 | forward 3 | TM | N in |
| 109 | LG:481436.5:2000FEB18 | 1164 | 1241 | forward 3 | TM | N in |
| 110 | LI:793701.1:2000FEB01 | 352 | 405 | forward 1 | TM | N in |
| 110 | LI:793701.1:2000FEB01 | 389 | 475 | forward 2 | TM | N in |
| 111 | LI:373637.1:2000FEB01 | 412 | 498 | forward 1 | TM | |
| 111 | LI:373637.1:2000FEB01 | 434 | 520 | forward 2 | TM | N out |
| 111 | LI:373637.1:2000FEB01 | 866 | 919 | forward 2 | TM | N out |
| 111 | LI:373637.1:2000FEB01 | 423 | 473 | forward 3 | TM | N in |
| 111 | LI:373637.1:2000FEB01 | 867 | 920 | forward 3 | TM | N in |
| 112 | LG:239368.2:2000MAY19 | 241 | 327 | forward 1 | TM | N out |
| 113 | LI:053825.1:2000MAY01 | 31 | 117 | forward 1 | TM | N out |
| 113 | LI:053826.1:2000MAY01 | 1102 | 1188 | forward 1 | TM | N out |
| 113 | LI:053826.1:2000MAY01 | 1282 | 1350 | forward 1 | TM | N out |
| 113 | LI:053826.1:2000MAY01 | 41 | 112 | forward 2 | TM | N out |
| 113 | LI:053826.1:2000MAY01 | 164 | 238 | forward 2 | TM | N out |
| 113 | LI:053826.1:2000MAY01 | 461 | 538 | forward 2 | TM | N out |
| 113 | LI:053826.1:2000MAY01 | 1130 | 1192 | forward 2 | TM | N out |
| 113 | LI:053826.1:2000MAY01 | 1214 | 1276 | forward 2 | TM | N out |
| 113 | LI:053826.1:2000MAY01 | 1307 | 1378 | forward 2 | TM | N out |
| 113 | LI:053826.1:2000MAY01 | 126 | 200 | forward 3 | TM | N in |
| 113 | LI:053826.1:2000MAY01 | 348 | 416 | forward 3 | TM | N in |
| 113 | LI:053826.1:2000MAY01 | 624 | 683 | forward 3 | TM | N in |
| 113 | LI:053826.1:2000MAY01 | 1215 | 1277 | forward 3 | TM | N in |
| 113 | LI:053826.1:2000MAY01 | 1290 | 1352 | forward 3 | TM | N in |
| 115 | LI:1071427.96:2000MAY01 | 1072 | 1140 | forward 1 | TM | |
| 115 | LI:1071427.96:2000MAY01 | 1297 | 1383 | forward 1 | TM | |
| 115 | LI:1071427.96:2000MAY01 | 1459 | 1536 | forward 1 | TM | |
| 115 | LI:1071427.96:2000MAY01 | 1765 | 1851 | forward 1 | TM | |
| 115 | LI:1071427.96:2000MAY01 | 1909 | 1971 | forward 1 | TM | |
| 115 | LI:1071427.96:2000MAY01 | 2002 | 2064 | forward 1 | TM | |
| 115 | LI:1071427.96:2000MAY01 | 1562 | 1648 | forward 2 | TM | N out |
| 115 | LI:1071427.96:2000MAY01 | 1706 | 1792 | forward 2 | TM | N out |
| 115 | LI:1071427.96:2000MAY01 | 1823 | 1885 | forward 2 | TM | N out |
| 115 | LI:1071427.96:2000MAY01 | 1913 | 1975 | forward 2 | TM | N out |
| 115 | LI:1071427.96:2000MAY01 | 2045 | 2098 | forward 2 | TM | N out |
| 115 | LI:1071427.96:2000MAY01 | 384 | 470 | forward 3 | TM | N out |
| 115 | LI:1071427.96:2000MAY01 | 840 | 926 | forward 3 | TM | N out |
| 115 | LI:1071427.96:2000MAY01 | 987 | 1049 | forward 3 | TM | N out |
| 115 | LI:1071427.96:2000MAY01 | 1092 | 1154 | forward 3 | TM | N out |
| 115 | LI:1071427.96:2000MAY01 | 1383 | 1454 | forward 3 | TM | N out |
| 115 | LI:1071427.96:2000MAY01 | 1599 | 1655 | forward 3 | TM | N out |
| 115 | LI:1071427.96:2000MAY01 | 1767 | 1844 | forward 3 | TM | N out |
| 115 | LI:1071427.96:2000MAY01 | 1884 | 1952 | forward 3 | TM | N out |
| 115 | LI:1071427.96:2000MAY01 | 2013 | 2099 | forward 3 | TM | N out |
| 115 | LI:1071427.96:2000MAY01 | 2127 | 2189 | forward 3 | TM | N out |
| 116 | LI:336338.8:2000MAY01 | 100 | 186 | forward 1 | TM | N out |
| 116 | LI:336338.8:2000MAY01 | 427 | 513 | forward 1 | TM | N out |
| 116 | LI:336338.8:2000MAY01 | 110 | 196 | forward 2 | TM | |
| 116 | LI:336338.8:2000MAY01 | 281 | 367 | forward 2 | TM | |
| 116 | LI:336338.8:2000MAY01 | 422 | 508 | forward 2 | TM | |
| 116 | LI:336338.8:2000MAY01 | 354 | 416 | forward 3 | TM | N out |
| 116 | LI:336338.8:2000MAY01 | 432 | 494 | forward 3 | TM | N out |
| 117 | LG:345527.1:2000FEB18 | 46 | 120 | forward 1 | TM | N out |
| 117 | LG:345527.1:2000FEB18 | 917 | 979 | forward 2 | TM | N out |
| 117 | LG:345527.1:2000FEB18 | 1010 | 1072 | forward 2 | TM | N out |
| 117 | LG:345527.1:2000FEB18 | 1112 | 1198 | forward 2 | TM | N out |
| 117 | LG:345527.1:2000FEB18 | 96 | 182 | forward 3 | TM | N out |
| 117 | LG:345527.1:2000FEB18 | 474 | 536 | forward 3 | TM | N out |
| 117 | LG:345527.1:2000FEB18 | 552 | 614 | forward 3 | TM | N out |
| 118 | LG:1089383.1:2000FEB18 | 43 | 126 | forward 1 | TM | N out |
| 118 | LG:1089383.1:2000FEB18 | 14 | 100 | forward 2 | TM | |
| 118 | LG:1089383.1:2000FEB18 | 140 | 205 | forward 2 | TM | |
| 118 | LG:1089383.1:2000FEB18 | 12 | 59 | forward 3 | TM | N out |
| 120 | LG:1093216.1:2000FEB18 | 31 | 117 | forward 1 | TM | N out |
| 120 | LG:1093216.1:2000FEB18 | 151 | 234 | forward 1 | TM | N out |
| 120 | LG:1093216.1:2000FEB18 | 283 | 348 | forward 1 | TM | N out |
| 120 | LG:1093216.1:2000FEB18 | 23 | 109 | forward 2 | TM | N in |
| 120 | LG:1093216.1:2000FEB18 | 143 | 193 | forward 2 | TM | N in |
| 120 | LG:1093216.1:2000FEB18 | 48 | 122 | forward 3 | TM | N out |
| 120 | LG:1093216.1:2000FEB18 | 180 | 263 | forward 3 | TM | N out |
| 122 | LI:335671.2:2000FEB01 | 22 | 108 | forward 1 | TM | N out |
| 122 | LI:335671.2:2000FEB01 | 1048 | 1134 | forward 1 | TM | N out |
| 122 | LI:335671.2:2000FEB01 | 854 | 916 | forward 2 | TM | N in |
| 122 | LI:335671.2:2000FEB01 | 926 | 988 | forward 2 | TM | N in |
| 122 | LI:335671.2:2000FEB01 | 998 | 1072 | forward 2 | TM | N in |
| 122 | LI:335671.2:2000FEB01 | 399 | 461 | forward 3 | TM | N out |
| 122 | LI:335671.2:2000FEB01 | 480 | 542 | forward 3 | TM | N out |
| 122 | LI:335671.2:2000FEB01 | 576 | 662 | forward 3 | TM | N out |
| 122 | LI:335671.2:2000FEB01 | 1023 | 1085 | forward 3 | TM | N out |
| 122 | LI:335671.2:2000FEB01 | 1098 | 1160 | forward 3 | TM | N out |
| 122 | LI:335671.2:2000FEB01 | 1173 | 1235 | forward 3 | TM | N out |
| 123 | LI:793758.1:2000FEB01 | 31 | 117 | forward 1 | TM | N out |
| 123 | LI:793758.1:2000FEB01 | 151 | 234 | forward 1 | TM | N out |
| 123 | LI:793758.1:2000FEB01 | 283 | 348 | forward 1 | TM | N out |
| 123 | LI:793758.1:2000FEB01 | 23 | 109 | forward 2 | TM | N in |
| 123 | LI:793758.1:2000FEB01 | 143 | 193 | forward 2 | TM | N in |
| 123 | LI:793758.1:2000FEB01 | 48 | 122 | forward 3 | TM | N out |
| 123 | LI:793758.1:2000FEB01 | 180 | 263 | forward 3 | TM | N out |
| 124 | LI:803718.1:2000FEB01 | 43 | 126 | forward 1 | TM | N out |
| 124 | LI:803718.1:2000FEB01 | 14 | 100 | forward 2 | TM | |
| 124 | LI:803718.1:2000FEB01 | 140 | 205 | forward 2 | TM | |
| 124 | LI:803718.1:2000FEB01 | 12 | 59 | forward 3 | TM | N out |
| 125 | LI:412179.1:2000FEB01 | 328 | 414 | forward 1 | TM | |
| 125 | LI:412179.1:2000FEB01 | 436 | 504 | forward 1 | TM | |
| 125 | LI:412179.1:2000FEB01 | 56 | 115 | forward 2 | TM | N out |
| 125 | LI:412179.1:2000FEB01 | 413 | 475 | forward 2 | TM | N out |
| 125 | LI:412179.1:2000FEB01 | 512 | 574 | forward 2 | TM | N out |
| 125 | LI:412179.1:2000FEB01 | 96 | 176 | forward 3 | TM | N out |
| 125 | LI:412179.1:2000FEB01 | 384 | 446 | forward 3 | TM | N out |
| 125 | LI:412179.1:2000FEB01 | 462 | 524 | forward 3 | TM | N out |
| 126 | LI:815679.1:2000FEB01 | 10 | 84 | forward 1 | TM | N out |
| 126 | LI:815679.1:2000FEB01 | 313 | 399 | forward 1 | TM | N out |
| 126 | LI:815679.1:2000FEB01 | 946 | 1032 | forward 1 | TM | N out |
| 126 | LI:815679.1:2000FEB01 | 1171 | 1248 | forward 1 | TM | N out |
| 126 | LI:815679.1:2000FEB01 | 323 | 409 | forward 2 | TM | N in |
| 126 | LI:815679.1:2000FEB01 | 500 | 568 | forward 2 | TM | N in |
| 126 | LI:815679.1:2000FEB01 | 971 | 1021 | forward 2 | TM | N in |
| 126 | LI:815679.1:2000FEB01 | 1493 | 1561 | forward 2 | TM | N in |
| 126 | LI:815679.1:2000FEB01 | 15 | 92 | forward 3 | TM | N in |
| 126 | LI:815679.1:2000FEB01 | 285 | 356 | forward 3 | TM | N in |
| 126 | LI:815679.1:2000FEB01 | 690 | 764 | forward 3 | TM | N in |
| 126 | LI:815679.1:2000FEB01 | 993 | 1076 | forward 3 | TM | N in |
| 126 | LI:815679.1:2000FEB01 | 1626 | 1712 | forward 3 | TM | N in |
| 127 | LI:481361.3:2000FEB01 | 199 | 252 | forward 1 | TM | N out |
| 128 | LG:247388.1:2000MAY19 | 190 | 240 | forward 1 | TM | N out |
| 128 | LG:247388.1:2000MAY19 | 233 | 319 | forward 2 | TM | N out |
| 128 | LG:247388.1:2000MAY19 | 446 | 532 | forward 2 | TM | N out |
| 130 | LI:787618.1:2000MAY01 | 10 | 84 | forward 1 | TM | N in |
| 130 | LI:787618.1:2000MAY01 | 313 | 399 | forward 1 | TM | N in |
| 130 | LI:787618.1:2000MAY01 | 679 | 750 | forward 1 | TM | N in |
| 130 | LI:787618.1:2000MAY01 | 1018 | 1098 | forward 1 | TM | N in |
| 130 | LI:787618.1:2000MAY01 | 1189 | 1266 | forward 1 | TM | N in |
| 130 | LI:787618.1:2000MAY01 | 323 | 409 | forward 2 | TM | N out |
| 130 | LI:787618.1:2000MAY01 | 500 | 568 | forward 2 | TM | N out |
| 130 | LI:787618.1:2000MAY01 | 944 | 1030 | forward 2 | TM | N out |
| 130 | LI:787618.1:2000MAY01 | 1508 | 1582 | forward 2 | TM | N out |
| 130 | LI:787618.1:2000MAY01 | 1616 | 1702 | forward 2 | TM | N out |
| 130 | LI:787618.1:2000MAY01 | 15 | 92 | forward 3 | TM | N out |
| 130 | LI:787618.1:2000MAY01 | 285 | 356 | forward 3 | TM | N out |
| 131 | LI:331610.2:2000MAY01 | 91 | 156 | forward 1 | TM | |
| 131 | LI:331610.2:2000MAY01 | 277 | 363 | forward 1 | TM | |
| 131 | LI:331610.2:2000MAY01 | 682 | 744 | forward 1 | TM | |
| 131 | LI:331610.2:2000MAY01 | 4126 | 4212 | forward 1 | TM | |
| 131 | LI:331610.2:2000MAY01 | 4951 | 5001 | forward 1 | TM | |
| 131 | LI:331610.2:2000MAY01 | 5023 | 5109 | forward 1 | TM | |
| 131 | LI:331610.2:2000MAY01 | 5128 | 5190 | forward 1 | TM | |
| 131 | LI:331610.2:2000MAY01 | 5407 | 5469 | forward 1 | TM | |
| 131 | LI:331610.2:2000MAY01 | 5485 | 5547 | forward 1 | TM | |
| 131 | LI:331610.2:2000MAY01 | 5563 | 5625 | forward 1 | TM | |
| 131 | LI:331610.2:2000MAY01 | 5728 | 5805 | forward 1 | TM | |
| 131 | LI:331610.2:2000MAY01 | 5896 | 5949 | forward 1 | RTM | |
| 131 | LI:331610.2:2000MAY01 | 6268 | 6327 | forward 1 | TM | |
| 131 | LI:331610.2:2000MAY01 | 6454 | 6522 | forward 1 | TM | |
| 131 | LI:331610.2:2000MAY01 | 6559 | 6645 | forward 1 | TM | |
| 131 | LI:331610.2:2000MAY01 | 7477 | 7539 | forward 1 | TM | |
| 131 | LI:331610.2:2000MAY01 | 7552 | 7614 | forward 1 | TM | |
| 131 | LI:331610.2:2000MAY01 | 671 | 724 | forward 2 | TM | N out |
| 131 | LI:331610.2:2000MAY01 | 4127 | 4213 | forward 2 | TM | N out |
| 131 | LI:331610.2:2000MAY01 | 4928 | 5011 | forward 2 | TM | N out |
| 131 | LI:331610.2:2000MAY01 | 5051 | 5113 | forward 2 | TM | N out |
| 131 | LI:331610.2:2000MAY01 | 5135 | 5197 | forward 2 | TM | N out |
| 131 | LI:331610.2:2000MAY01 | 5207 | 5269 | forward 2 | TM | N out |
| 131 | LI:331610.2:2000MAY01 | 5537 | 5611 | forward 2 | TM | N out |
| 131 | LI:331610.2:2000MAY01 | 5726 | 5797 | forward 2 | TM | N out |
| 131 | LI:331610.2:2000MAY01 | 5903 | 5989 | forward 2 | TM | N out |
| 131 | LI:331610.2:2000MAY01 | 6392 | 6478 | forward 2 | TM | N out |
| 131 | LI:331610.2:2000MAY01 | 6746 | 6814 | forward 2 | TM | N out |
| 131 | LI:331610.2:2000MAY01 | 7295 | 7381 | forward 2 | TM | N out |
| 131 | LI:331610.2:2000MAY01 | 7586 | 7633 | forward 2 | TM | N out |
| 131 | LI:331610.2:2000MAY01 | 2763 | 2849 | forward 3 | TM | |
| 131 | LI:331610.2:2000MAY01 | 4527 | 4595 | forward 3 | TM | |
| 131 | LI:331610.2:2000MAY01 | 5079 | 5165 | forward 3 | TM | |
| 131 | LI:331610.2:2000MAY01 | 5445 | 5516 | forward 3 | TM | |
| 131 | LI:331610.2:2000MAY01 | 5676 | 5759 | forward 3 | TM | |
| 131 | LI:331610.2:2000MAY01 | 6255 | 6341 | forward 3 | TM | |
| 131 | LI:331610.2:2000MAY01 | 6378 | 6464 | forward 3 | TM | |
| 131 | LI:331610.2:2000MAY01 | 6624 | 6692 | forward 3 | TM | |
| 131 | LI:331610.2:2000MAY01 | 6705 | 6779 | forward 3 | TM | |
| 131 | LI:331610.2:2000MAY01 | 6810 | 6884 | forward 3 | TM | |
| 131 | LI:331610.2:2000MAY01 | 7062 | 7133 | forward 3 | TM | |
| 131 | LI:331610.2:2000MAY01 | 7677 | 7748 | forward 3 | TM | |
| 131 | LI:331610.2:2000MAY01 | 7833 | 7919 | forward 3 | TM | |
| 132 | LG:982697.1:2000FEB18 | 355 | 441 | forward 1 | TM | N in |
| 132 | LG:982697.1:2000FEB18 | 946 | 993 | forward 1 | TM | N in |
| 132 | LG:982697.1:2000FEB18 | 897 | 983 | forward 3 | TM | N in |
| 132 | LG:982697.1:2000FEB18 | 1215 | 1301 | forward 3 | TM | N in |
| 133 | LG:1080896.1:2000FEB18 | 367 | 426 | forward 1 | TM | N in |
| 133 | LG:1080896.1:2000FEB18 | 476 | 562 | forward 2 | TM | N in |
| 133 | LG:1080896.1:2000FEB18 | 815 | 901 | forward 2 | TM | N in |
| 133 | LG:1080896.1:2000FEB18 | 342 | 395 | forward 3 | TM | N in |
| 134 | LI:811341.1:2000FEB01 | 562 | 615 | forward 1 | TM | N out |
| 134 | LI:811341.1:2000FEB01 | 691 | 777 | forward 1 | TM | N out |
| 135 | LI:903225.1:2000FEB01 | 20 | 100 | forward 2 | TM | N out |
| 135 | LI:903225.1:2000FEB01 | 12 | 83 | forward 3 | TM | N out |
| 135 | LI:903225.1:2000FEB01 | 768 | 827 | forward 3 | TM | N out |
| 137 | LG:979580.1:2000MAY19 | 298 | 354 | forward 1 | TM | N in |
| 137 | LG:979580.1:2000MAY19 | 826 | 909 | forward 1 | TM | N in |
| 137 | LG:979580.1:2000MAY19 | 934 | 1020 | forward 1 | TM | N in |
| 137 | LG:979580.1:2000MAY19 | 233 | 289 | forward 2 | TM | N out |
| 137 | LG:979580.1:2000MAY19 | 338 | 418 | forward 2 | TM | N out |
| 137 | LG:979580.1:2000MAY19 | 201 | 272 | forward 3 | TM | N in |
| 138 | LI:1169865.1:2000MAY01 | 197 | 283 | forward 2 | TM | N in |
| 138 | LI:1169865.1:2000MAY01 | 863 | 949 | forward 2 | TM | N in |
| 139 | LG:337818.2:2000FEB18 | 40 | 117 | forward 1 | TM | N out |
| 139 | LG:337818.2:2000FEB18 | 532 | 618 | forward 1 | TM | N out |
| 139 | LG:337818.2:2000FEB18 | 907 | 993 | forward 1 | TM | N out |
| 139 | LG:337818.2:2000FEB18 | 1372 | 1425 | forward 1 | TM | N out |
| 140 | LI:337818.1:2000FEB01 | 40 | 114 | forward 1 | TM | N in |
| 140 | LI:337818.1:2000FEB01 | 401 | 466 | forward 2 | TM | N in |
| 140 | LI:337818.1:2000FEB01 | 852 | 905 | forward 3 | TM | N in |
| 141 | LG:241577.4:2000MAY19 | 496 | 582 | forward 1 | TM | N in |
| 142 | LG:344786.4:2000MAY19 | 19 | 105 | forward 1 | TM | N out |
| 142 | LG:344786.4:2000MAY19 | 14 | 88 | forward 2 | TM | N in |
| 142 | LG:344786.4:2000MAY19 | 173 | 247 | forward 2 | TM | N in |
| 142 | LG:344786.4:2000MAY19 | 21 | 107 | forward 3 | TM | |
| 143 | LI:414307.1:2000FEB01 | 116 | 202 | forward 2 | TM | N in |
| 144 | LI:202943.2:2000FEB01 | 166 | 237 | forward 1 | TM | N in |
| 144 | LI:202943.2:2000FEB01 | 263 | 313 | forward 2 | TM | N out |
| 144 | LI:202943.2:2000FEB01 | 276 | 326 | forward 3 | TM | N in |
| 146 | LI:815961.1:2000FEB01 | 232 | 291 | forward 1 | TM | N out |
| 146 | LI:815961.1:2000FEB01 | 81 | 167 | forward 3 | TM | N out |
| 146 | LI:815961.1:2000FEB01 | 243 | 329 | forward 3 | TM | N out |
| 146 | LI:815961.1:2000FEB01 | 354 | 422 | forward 3 | TM | N out |
| 146 | LI:815961.1:2000FEB01 | 573 | 659 | forward 3 | TM | N out |
| 146 | LI:815961.1:2000FEB01 | 741 | 803 | forward 3 | TM | N out |
| 147 | LG:120744.1:2000MAY19 | 181 | 249 | forward 1 | TM | N out |
| 147 | LG:120744.1:2000MAY19 | 188 | 256 | forward 2 | TM | |
| 147 | LG:120744.1:2000MAY19 | 275 | 328 | forward 2 | TM | |
| 148 | LI:757520.1:2000MAY01 | 2140 | 2220 | forward 1 | TM | N in |
| 148 | LI:757520.1:2000MAY01 | 2293 | 2379 | forward 1 | TM | N in |
| 148 | LI:757520.1:2000MAY01 | 1988 | 2059 | forward 2 | TM | N in |
| 148 | LI:757520.1:2000MAY01 | 2285 | 2359 | forward 2 | TM | N in |
| 148 | LI:757520.1:2000MAY01 | 1677 | 1763 | forward 3 | TM | |
| 148 | LI:757520.1:2000MAY01 | 1995 | 2066 | forward 3 | TM | |
| 149 | LG:160570.1:2000FEB18 | 345 | 413 | forward 3 | TM | N out |
| 149 | LG:160570.1:2000FEB18 | 462 | 518 | forward 3 | TM | N out |
| 151 | LI:221285.1:2000FEB01 | 1375 | 1452 | forward 1 | TM | N out |
| 152 | LI:401605.2:2000FEB01 | 235 | 321 | forward 1 | TM | N in |
| 152 | LI:401605.2:2000FEB01 | 192 | 263 | forward 3 | TM | N in |
| 152 | LI:401605.2:2000FEB01 | 489 | 563 | forward 3 | TM | N in |
| 153 | LI:329017.1:2000FEB01 | 179 | 235 | forward 2 | TM | N in |
| 153 | LI:329017.1:2000FEB01 | 359 | 433 | forward 2 | TM | N in |
| 153 | LI:329017.1:2000FEB01 | 449 | 526 | forward 2 | TM | N in |
| 153 | LI:329017.1:2000FEB01 | 617 | 703 | forward 2 | TM | N in |
| 153 | LI:329017.1:2000FEB01 | 920 | 973 | forward 2 | TM | N in |
| 155 | LG:403409.1:2000MAY19 | 136 | 222 | forward 1 | TM | N out |
| 155 | LG:403409.1:2000MAY19 | 973 | 1029 | forward 1 | TM | N out |
| 155 | LG:403409.1:2000MAY19 | 1285 | 1371 | forward 1 | TM | N out |
| 155 | LG:403409.1:2000MAY19 | 182 | 268 | forward 2 | TM | N in |
| 156 | LG:233933.5:2000MAY19 | 148 | 234 | forward 1 | TM | N out |
| 156 | LG:233933.5:2000MAY19 | 39 | 125 | forward 3 | TM | N out |
| 157 | LI:290344.1:2000MAY01 | 232 | 312 | forward 1 | TM | N out |
| 157 | LI:290344.1:2000MAY01 | 1258 | 1311 | forward 1 | TM | N out |
| 157 | LI:290344.1:2000MAY01 | 3640 | 3714 | forward 1 | TM | N out |
| 157 | LI:290344.1:2000MAY01 | 4366 | 4449 | forward 1 | TM | N out |
| 157 | LI:290344.1:2000MAY01 | 4468 | 4548 | forward 1 | TM | N out |
| 157 | LI:290344.1:2000MAY01 | 146 | 226 | forward 2 | TM | N out |
| 157 | LI:290344.1:2000MAY01 | 3122 | 3196 | forward 2 | TM | N out |
| 157 | LI:290344.1:2000MAY01 | 3833 | 3919 | forward 2 | TM | N out |
| 157 | LI:290344.1:2000MAY01 | 4457 | 4537 | forward 2 | TM | N out |
| 157 | LI:290344.1:2000MAY01 | 4760 | 4846 | forward 2 | TM | N out |
| 157 | LI:290344.1:2000MAY01 | 432 | 503 | forward 3 | TM | N out |
| 157 | LI:290344.1:2000MAY01 | 1647 | 1733 | forward 3 | TM | N out |
| 157 | LI:290344.1:2000MAY01 | 3177 | 3248 | forward 3 | TM | N out |
| 157 | LI:290344.1:2000MAY01 | 3594 | 3680 | forward 3 | TM | N out |
| 157 | LI:290344.1:2000MAY01 | 3753 | 3815 | forward 3 | TM | N out |
| 157 | LI:290344.1:2000MAY01 | 3864 | 3926 | forward 3 | TM | N out |
| 157 | LI:290344.1:2000MAY01 | 4443 | 4526 | forward 3 | TM | N out |
| 158 | LI:410742.1:2000MAY01 | 136 | 210 | forward 1 | TM | N out |
| 158 | LI:410742.1:2000MAY01 | 2200 | 2286 | forward 1 | TM | N out |
| 158 | LI:410742.1:2000MAY01 | 2437 | 2514 | forward 1 | TM | N out |
| 158 | LI:410742.1:2000MAY01 | 3149 | 3229 | forward 2 | TM | N in |
| 158 | LI:410742.1:2000MAY01 | 3437 | 3505 | forward 2 | TM | N in |
| 158 | LI:410742.1:2000MAY01 | 510 | 578 | forward 3 | TM | N in |
| 158 | LI:410742.1:2000MAY01 | 1905 | 1991 | forward 3 | TM | N in |
| 158 | LI:410742.1:2000MAY01 | 2811 | 2897 | forward 3 | TM | N in |
| 158 | LI:410742.1:2000MAY01 | 3168 | 3254 | forward 3 | TM | N in |
| 159 | LG:406568.1:2000MAY19 | 490 | 549 | forward 1 | TM | N in |
| 159 | LG:406568.1:2000MAY19 | 1732 | 1818 | forward 1 | TM | N in |
| 159 | LG:406568.1:2000MAY19 | 1825 | 1899 | forward 1 | TM | N in |
| 159 | LG:406568.1:2000MAY19 | 1918 | 2004 | forward 1 | TM | N in |
| 159 | LG:406568.1:2000MAY19 | 12 | 59 | forward 3 | TM | N in |
| 159 | LG:406568.1:2000MAY19 | 1935 | 2018 | forward 3 | TM | N in |
| 159 | LG:406568.1:2000MAY19 | 2094 | 2174 | forward 3 | TM | N in |
| 160 | LI:283762.1:2000MAY01 | 1675 | 1746 | forward 1 | TM | |
| 160 | LI:283762.1:2000MAY01 | 2095 | 2181 | forward 1 | TM | |
| 160 | LI:283762.1:2000MAY01 | 2632 | 2718 | forward 1 | TM | |
| 160 | LI:283762.1:2000MAY01 | 2830 | 2916 | forward 1 | TM | |
| 160 | LI:283762.1:2000MAY01 | 2941 | 3027 | forward 1 | TM | |
| 160 | LI:283762.1:2000MAY01 | 3235 | 3321 | forward 1 | TM | |
| 160 | LI:283762.1:2000MAY01 | 3328 | 3414 | forward 1 | TM | |
| 160 | LI:283762.1:2000MAY01 | 3592 | 3666 | forward 1 | TM | |
| 160 | LI:283762.1:2000MAY01 | 3682 | 3768 | forward 1 | TM | |
| 160 | LI:283762.1:2000MAY01 | 4153 | 4224 | forward 1 | TM | |
| 160 | LI:283762.1:2000MAY01 | 4360 | 4434 | forward 1 | TM | |
| 160 | LI:283762.1:2000MAY01 | 4594 | 4656 | forward 1 | TM | |
| 160 | LI:283762.1:2000MAY01 | 4681 | 4743 | forward 1 | TM | |
| 160 | LI:283762.1:2000MAY01 | 4885 | 4962 | forward 1 | TM | |
| 160 | LI:283762.1:2000MAY01 | 5011 | 5061 | forward 1 | TM | |
| 160 | LI:283762.1:2000MAY01 | 92 | 178 | forward 2 | TM | N in |
| 160 | LI:283762.1:2000MAY01 | 278 | 364 | forward 2 | TM | N in |
| 160 | LI:283762.1:2000MAY01 | 995 | 1075 | forward 2 | TM | N in |
| 160 | LI:283762.1:2000MAY01 | 1523 | 1597 | forward 2 | TM | N in |
| 160 | LI:283762.1:2000MAY01 | 1817 | 1903 | forward 2 | TM | N in |
| 160 | LI:283762.1:2000MAY01 | 2522 | 2599 | forward 2 | TM | N in |
| 160 | LI:283762.1:2000MAY01 | 2666 | 2752 | forward 2 | TM | N in |
| 160 | LI:283762.1:2000MAY01 | 2837 | 2887 | forward 2 | TM | N in |
| 160 | LI:283762.1:2000MAY01 | 3038 | 3097 | forward 2 | TM | N in |
| 160 | LI:283762.1:2000MAY01 | 3563 | 3625 | forward 2 | TM | N in |
| 160 | LI:283762.1:2000MAY01 | 3638 | 3700 | forward 2 | TM | N in |
| 160 | LI:283762.1:2000MAY01 | 4067 | 4144 | forward 2 | TM | N in |
| 160 | LI:283762.1:2000MAY01 | 4439 | 4522 | forward 2 | TM | N in |
| 160 | LI:283762.1:2000MAY01 | 4685 | 4765 | forward 2 | TM | N in |
| 160 | LI:283762.1:2000MAY01 | 4784 | 4843 | forward 2 | TM | N in |
| 160 | LI:283762.1:2000MAY01 | 4973 | 5050 | forward 2 | TM | N in |
| 160 | LI:283762.1:2000MAY01 | 5072 | 5125 | forward 2 | TM | N in |
| 160 | LI:283762.1:2000MAY01 | 693 | 755 | forward 3 | TM | N out |
| 160 | LI:283762.1:2000MAY01 | 765 | 827 | forward 3 | TM | N out |
| 160 | LI:283762.1:2000MAY01 | 840 | 902 | forward 3 | TM | N out |
| 160 | LI:283762.1:2000MAY01 | 1623 | 1694 | forward 3 | TM | N out |
| 160 | LI:283762.1:2000MAY01 | 1800 | 1880 | forward 3 | TM | N out |
| 160 | LI:283762.1:2000MAY01 | 2622 | 2708 | forward 3 | TM | N out |
| 160 | LI:283762.1:2000MAY01 | 2778 | 2861 | forward 3 | TM | N out |
| 160 | LI:283762.1:2000MAY01 | 3144 | 3230 | forward 3 | TM | N out |
| 160 | LI:283762.1:2000MAY01 | 3276 | 3362 | forward 3 | TM | N out |
| 160 | LI:283762.1:2000MAY01 | 3441 | 3527 | forward 3 | TM | N out |
| 160 | LI:283762.1:2000MAY01 | 3666 | 3752 | forward 3 | TM | N out |
| 160 | LI:283762.1:2000MAY01 | 4077 | 4163 | forward 3 | TM | N out |
| 160 | LI:283762.1:2000MAY01 | 4245 | 4331 | forward 3 | TM | N out |
| 160 | LI:283762.1:2000MAY01 | 4395 | 4481 | forward 3 | TM | N out |
| 160 | LI:283762.1:2000MAY01 | 4584 | 4646 | forward 3 | TM | N out |
| 160 | LI:283762.1:2000MAY01 | 4662 | 4724 | forward 3 | TM | N out |
| 160 | LI:283762.1:2000MAY01 | 4845 | 4892 | forward 3 | TM | N out |
| 161 | LI:347687.113:2000MAY01 | 319 | 405 | forward 1 | TM | N out |
| 161 | LI:347687.113:2000MAY01 | 463 | 549 | forward 1 | TM | N out |
| 161 | LI:347687.113:2000MAY01 | 733 | 819 | forward 1 | TM | N out |
| 161 | LI:347687.113:2000MAY01 | 1240 | 1293 | forward 1 | TM | N out |
| 161 | LI:347687.113:2000MAY01 | 1720 | 1797 | forward 1 | TM | N out |
| 161 | LI:347687.113:2000MAY01 | 1861 | 1908 | forward 1 | TM | N out |
| 161 | LI:347687.113:2000MAY01 | 1972 | 2034 | forward 1 | TM | N out |
| 161 | LI:347687.113:2000MAY01 | 2050 | 2112 | forward 1 | TM | N out |
| 161 | LI:347687.113:2000MAY01 | 2308 | 2394 | forward 1 | TM | N out |
| 161 | LI:347687.113:2000MAY01 | 977 | 1057 | forward 2 | TM | N in |
| 161 | LI:347687.113:2000MAY01 | 1250 | 1309 | forward 2 | TM | N in |
| 161 | LI:347687.113:2000MAY01 | 1730 | 1792 | forward 2 | TM | N in |
| 161 | LI:347687.113:2000MAY01 | 1808 | 1870 | forward 2 | TM | N in |
| 161 | LI:347687.113:2000MAY01 | 1886 | 1948 | forward 2 | TM | N in |
| 161 | LI:347687.113:2000MAY01 | 324 | 398 | forward 3 | TM | N in |
| 161 | LI:347687.113:2000MAY01 | 948 | 1034 | forward 3 | TM | N in |
| 161 | LI:347687.113:2000MAY01 | 1686 | 1763 | forward 3 | TM | N in |
| 161 | LI:347687.113:2000MAY01 | 1791 | 1874 | forward 3 | TM | N in |
| 161 | LI:347687.113:2000MAY01 | 2025 | 2108 | forward 3 | TM | N in |
| 163 | LG:451710.1:2000FEB18 | 502 | 588 | forward 1 | TM | N in |
| 163 | LG:451710.1:2000FEB18 | 453 | 515 | forward 3 | TM | N in |
| 164 | LG:455771.1:2000FEB18 | 199 | 285 | forward 1 | TM | N out |
| 165 | LG:452089.1:2000FEB18 | 695 | 772 | forward 2 | TM | N out |
| 165 | LG:452089.1:2000FEB18 | 708 | 764 | forward 3 | TM | N out |
| 166 | LG:246415.1:2000FEB18 | 196 | 246 | forward 1 | TM | N in |
| 167 | LG:414144.10:2000FEB18 | 589 | 672 | forward 1 | TM | N in |
| 167 | LG:414144.10:2000FEB18 | 615 | 692 | forward 3 | TM | N out |
| 168 | LG:1101445.1:2000FEB18 | 787 | 858 | forward 1 | TM | N out |
| 168 | LG:1101445.1:2000FEB18 | 506 | 592 | forward 2 | TM | N out |
| 169 | LG:452134.1:2000FEB18 | 276 | 326 | forward 3 | TM | N out |
| 170 | LI:903021.1:2000FEB01 | 109 | 162 | forward 1 | TM | N out |
| 172 | LG:449404.1:2000MAY19 | 163 | 219 | forward 1 | TM | N out |
| 172 | LG:449404.1:2000MAY19 | 200 | 280 | forward 2 | TM | N out |
| 173 | LG:449413.1:2000MAY19 | 353 | 439 | forward 2 | TM | N out |
| 177 | LG:1101153.1:2000MAY19 | 520 | 600 | forward 1 | TM | N in |
| 177 | LG:1101153.1:2000MAY19 | 585 | 671 | forward 3 | TM | N in |
| 178 | LI:257695.20:2000MAY01 | 433 | 516 | forward 1 | TM | N in |
| 179 | LI:455771.1:2000MAY01 | 199 | 285 | forward 1 | TM | N out |
| 180 | LI:274551.1:2000MAY01 | 81 | 152 | forward 3 | TM | N out |
| 180 | LI:274551.1:2000MAY01 | 216 | 269 | forward 3 | TM | N out |
| 181 | LI:035973.1:2000MAY01 | 622 | 708 | forward 1 | TM | N out |
| 181 | LI:035973.1:2000MAY01 | 596 | 682 | forward 2 | TM | N out |
| 181 | LI:035973.1:2000MAY01 | 588 | 674 | forward 3 | TM | N out |
| 182 | LG:978427.5:2000FEB18 | 221 | 295 | forward 2 | TM | N out |
| 182 | LG:978427.5:2000FEB18 | 365 | 433 | forward 2 | TM | N out |
| 182 | LG:978427.5:2000FEB18 | 198 | 284 | forward 3 | TM | N out |
| 183 | LG:247781.2:2000FEB18 | 22 | 108 | forward 1 | TM | N in |
| 183 | LG:247781.2:2000FEB18 | 1114 | 1200 | forward 1 | TM | N in |
| 183 | LG:247781.2:2000FEB18 | 1149 | 1235 | forward 3 | TM | N in |
| 185 | LI:333307.2:2000FEB01 | 24 | 98 | forward 3 | TM | N out |
| 187 | LG:414732.1:2000MAY19 | 40 | 93 | forward 1 | TM | N out |
| 187 | LG:414732.1:2000MAY19 | 156 | 233 | forward 3 | TM | N out |
| 188 | LG:413910.6:2000MAY19 | 385 | 441 | forward 1 | TM | N out |
| 188 | LG:413910.6:2000MAY19 | 886 | 948 | forward 1 | TM | N out |
| 188 | LG:413910.6.2000MAY19 | 104 | 190 | forward 2 | TM | N out |
| 188 | LG:413910.6:2000MAY19 | 387 | 461 | forward 3 | TM | N out |
| 188 | LG:413910.6:2000MAY19 | 921 | 1007 | forward 3 | TM | N out |
| 189 | LI:414732.2:2000MAY01 | 34 | 93 | forward 1 | TM | N out |
| 189 | LI:414732.2:2000MAY01 | 24 | 110 | forward 3 | TM | N out |
| 189 | LI:414732.2:2000MAY01 | 159 | 236 | forward 3 | TM | N out |
| 190 | LI:900264.2:2000MAY01 | 730 | 807 | forward 1 | TM | N in |
| 190 | LI:900264.2:2000MAY01 | 1018 | 1092 | forward 1 | TM | N in |
| 190 | LI:900264.2:2000MAY01 | 1294 | 1350 | forward 1 | TM | N in |
| 190 | LI:900264.2:2000MAY01 | 1519 | 1578 | forward 1 | TM | N in |
| 190 | LI:900264.2:2000MAY01 | 2311 | 2397 | forward 1 | TM | N in |
| 190 | LI:900264.2:2000MAY01 | 2509 | 2562 | forward 1 | TM | N in |
| 190 | LI:900264.2:2000MAY01 | 2752 | 2808 | forward 1 | TM | N in |
| 190 | LI:900264.2:2000MAY01 | 3103 | 3165 | forward 1 | TM | N in |
| 190 | LI:900264.2:2000MAY01 | 3178 | 3240 | forward 1 | TM | N in |
| 190 | LI:900264.2:2000MAY01 | 3253 | 3315 | forward 1 | TM | N in |
| 190 | LI:900264.2:2000MAY01 | 3424 | 3510 | forward 1 | TM | N in |
| 190 | LI:900264.2:2000MAY01 | 3520 | 3603 | forward 1 | TM | N in |
| 190 | LI:900264.2:2000MAY01 | 3883 | 3945 | forward 1 | TM | N in |
| 190 | LI:900264.2:2000MAY01 | 3982 | 4044 | forward 1 | TM | N in |
| 190 | LI:900264.2:2000MAY01 | 68 | 154 | forward 2 | TM | |
| 190 | LI:900264.2:2000MAY01 | 188 | 274 | forward 2 | TM | |
| 190 | LI:900264.2:2000MAY01 | 1079 | 1165 | forward 2 | TM | |
| 190 | LI:900264.2:2000MAY01 | 2285 | 2359 | forward 2 | TM | |
| 190 | LI:900264.2:2000MAY01 | 2732 | 2812 | forward 2 | TM | |
| 190 | LI:900264.2:2000MAY01 | 3095 | 3172 | forward 2 | TM | |
| 190 | LI:900264.2:2000MAY01 | 3260 | 3319 | forward 2 | TM | |
| 190 | LI:900264.2:2000MAY01 | 3434 | 3505 | forward 2 | TM | |
| 190 | LI:900264.2:2000MAY01 | 3515 | 3601 | forward 2 | TM | |
| 190 | LI:900264.2:2000MAY01 | 3662 | 3748 | forward 2 | TM | |
| 190 | LI:900264.2:2000MAY01 | 3842 | 3913 | forward 2 | TM | |
| 190 | LI:900264.2:2000MAY01 | 3992 | 4063 | forward 2 | TM | |
| 190 | LI:900264.2:2000MAY01 | 198 | 248 | forward 3 | TM | N in |
| 190 | LI:900264.2:2000MAY01 | 1080 | 1133 | forward 3 | TM | N in |
| 190 | LI:900264.2:2000MAY01 | 1431 | 1517 | forward 3 | TM | N in |
| 190 | LI:900264.2:2000MAY01 | 1518 | 1571 | forward 3 | TM | N in |
| 190 | LI:900264.2:2000MAY01 | 1740 | 1814 | forward 3 | TM | N in |
| 190 | LI:900264.2:2000MAY01 | 2409 | 2480 | forward 3 | TM | N in |
| 190 | LI:900264.2:2000MAY01 | 2928 | 2993 | forward 3 | TM | N in |
| 190 | LI:900264.2:2000MAY01 | 3096 | 3161 | forward 3 | TM | N in |
| 190 | LI:900264.2:2000MAY01 | 3342 | 3404 | forward 3 | TM | N in |
| 190 | LI:900264.2:2000MAY01 | 3447 | 3509 | forward 3 | TM | N in |
| 190 | LI:900264.2:2000MAY01 | 3531 | 3614 | forward 3 | TM | N in |
| 190 | LI:900264.2:2000MAY01 | 3987 | 4064 | forward 3 | TM | N in |
| 191 | LI:335593.1:2000MAY01 | 685 | 771 | forward 1 | TM | N in |
| 191 | LI:335593.1:2000MAY01 | 1273 | 1335 | forward 1 | TM | N in |
| 191 | LI:335593.1:2000MAY01 | 1366 | 1428 | forward 1 | TM | N in |
| 191 | LI:335593.1:2000MAY01 | 710 | 757 | forward 2 | TM | N in |
| 191 | LI:335593.1:2000MAY01 | 1250 | 1336 | forward 2 | TM | N in |
| 191 | LI:335593.1:2000MAY01 | 1358 | 1408 | forward 2 | TM | N in |
| 191 | LI:335593.1:2000MAY01 | 1448 | 1525 | forward 2 | TM | N in |
| 191 | LI:335593.1:2000MAY01 | 1604 | 1690 | forward 2 | TM | N in |
| 191 | LI:335593.1:2000MAY01 | 81 | 128 | forward 3 | TM | N in |
| 191 | LI:335593.1:2000MAY01 | 246 | 296 | forward 3 | TM | N in |
| 191 | LI:335593.1:2000MAY01 | 807 | 866 | forward 3 | TM | N in |
| 191 | LI:335593.1:2000MAY01 | 876 | 947 | forward 3 | TM | N in |
| 191 | LI:335593.1:2000MAY01 | 1155 | 1217 | forward 3 | TM | N in |
| 191 | LI:335593.1:2000MAY01 | 1233 | 1295 | forward 3 | TM | N in |
| 191 | LI:335593.1:2000MAY01 | 1359 | 1445 | forward 3 | TM | N in |
| 192 | LI:1189543.1:2000MAY01 | 1765 | 1842 | forward 1 | TM | |
| 192 | LI:1189543.1:2000MAY01 | 1861 | 1935 | forward 1 | TM | |
| 192 | LI:1189543.1:2000MAY01 | 2236 | 2307 | forward 1 | TM | |
| 192 | LI:1189543.1:2000MAY01 | 2356 | 2442 | forward 1 | TM | |
| 192 | LI:1189543.1:2000MAY01 | 2476 | 2544 | forward 1 | TM | |
| 192 | LI:1189543.1:2000MAY01 | 2659 | 2712 | forward 1 | TM | |
| 192 | LI:1189543.1:2000MAY01 | 3097 | 3174 | forward 1 | TM | |
| 192 | LI:1189543.1:2000MAY01 | 3217 | 3288 | forward 1 | TM | |
| 192 | LI:1189543.1:2000MAY01 | 3439 | 3492 | forward 1 | TM | |
| 192 | LI:1189543.1:2000MAY01 | 860 | 946 | forward 2 | TM | |
| 192 | LI:1189543.1:2000MAY01 | 1016 | 1099 | forward 2 | TM | |
| 192 | LI:1189543.1:2000MAY01 | 1145 | 1216 | forward 2 | TM | |
| 192 | LI:1189543.1:2000MAY01 | 1601 | 1672 | forward 2 | TM | |
| 192 | LI:1189543.1:2000MAY01 | 1691 | 1768 | forward 2 | TM | |
| 192 | LI:1189543.1:2000MAY01 | 2411 | 2485 | forward 2 | TM | |
| 192 | LI:1189543.1:2000MAY01 | 2831 | 2917 | forward 2 | TM | |
| 192 | LI:1189543.1:2000MAY01 | 3080 | 3166 | forward 2 | TM | |
| 192 | LI:1189543.1:2000MAY01 | 3227 | 3310 | forward 2 | TM | |
| 192 | LI:1189543.1:2000MAY01 | 1155 | 1229 | forward 3 | TM | N out |
| 192 | LI:1189543.1:2000MAY01 | 1683 | 1766 | forward 3 | TM | N out |
| 192 | LI:1189543.1:2000MAY01 | 1770 | 1838 | forward 3 | TM | N out |
| 192 | LI:1189543.1:2000MAY01 | 2019 | 2069 | forward 3 | TM | N out |
| 192 | LI:1189543.1:2000MAY01 | 2352 | 2438 | forward 3 | TM | N out |
| 192 | LI:1189543.1:2000MAY01 | 2508 | 2594 | forward 3 | TM | N out |
| 192 | LI:1189543.1:2000MAY01 | 3030 | 3101 | forward 3 | TM | N out |
| 192 | LI:1189543.1:2000MAY01 | 3183 | 3263 | forward 3 | TM | N out |
| 192 | LI:1189543.1:2000MAY01 | 3360 | 3446 | forward 3 | TM | N out |
| 193 | LG:455450.1:2000FEB18 | 422 | 490 | forward 2 | TM | N out |
| 194 | LG:1040978.1:2000FEB18 | 500 | 586 | forward 2 | TM | N out |
| 194 | LG:1040978.1:2000FEB18 | 276 | 332 | forward 3 | TM | N out |
| 196 | LG:132147.3:2000FEB18 | 259 | 345 | forward 1 | TM | N out |
| 196 | LG:132147.3:2000FEB18 | 418 | 504 | forward 1 | TM | N out |
| 196 | LG:132147.3:2000FEB18 | 718 | 780 | forward 1 | TM | N out |
| 196 | LG:132147.3:2000FEB18 | 1477 | 1548 | forward 1 | TM | N out |
| 196 | LG:132147.3:2000FEB18 | 1585 | 1647 | forward 1 | TM | N out |
| 196 | LG:132147.3:2000FEB18 | 1690 | 1752 | forward 1 | TM | N out |
| 196 | LG:132147.3:2000FEB18 | 2560 | 2637 | forward 1 | TM | N out |
| 196 | LG:132147.3:2000FEB18 | 2731 | 2790 | forward 1 | TM | N out |
| 196 | LG:132147.3:2000FEB18 | 2908 | 2976 | forward 1 | TM | N out |
| 196 | LG:132147.3:2000FEB18 | 3082 | 3168 | forward 1 | TM | N out |
| 196 | LG:132147.3:2000FEB18 | 3184 | 3243 | forward 1 | TM | N out |
| 196 | LG:132147.3:2000FEB18 | 3376 | 3462 | forward 1 | TM | N out |
| 196 | LG:132147.3:2000FEB18 | 1451 | 1531 | forward 2 | TM | N out |
| 196 | LG:132147.3:2000FEB18 | 1538 | 1615 | forward 2 | TM | N out |
| 196 | LG:132147.3:2000FEB18 | 2741 | 2827 | forward 2 | TM | N out |
| 196 | LG:132147.3:2000FEB18 | 2960 | 3031 | forward 2 | TM | N out |
| 196 | LG:132147.3:2000FEB18 | 3050 | 3112 | forward 2 | TM | N out |
| 196 | LG:132147.3:2000FEB18 | 1626 | 1703 | forward 3 | TM | N in |
| 196 | LG:132147.3:2000FEB18 | 2508 | 2594 | forward 3 | TM | N in |
| 196 | LG:132147.3:2000FEB18 | 2919 | 2987 | forward 3 | TM | N in |
| 196 | LG:132147.3:2000FEB18 | 3177 | 3263 | forward 3 | TM | N in |
| 196 | LG:132147.3:2000FEB18 | 3372 | 3422 | forward 3 | TM | N in |
| 197 | LI:036034.1:2000FEB01 | 157 | 219 | forward 1 | TM | N out |
| 197 | LI:036034.1:2000FEB01 | 395 | 457 | forward 2 | TM | N in |
| 197 | LI:036034.1:2000FEB01 | 479 | 541 | forward 2 | TM | N in |
| 197 | LI:036034.1:2000FEB01 | 563 | 625 | forward 2 | TM | N in |
| 197 | LI:036034.1:2000FEB01 | 647 | 709 | forward 2 | TM | N in |
| 198 | LG:162161.1:2000MAY19 | 372 | 458 | forward 3 | TM | N in |
| 199 | LG:407214.10:2000MAY19 | 34 | 120 | forward 1 | TM | N out |
| 199 | LG:407214.10:2000MAY19 | 44 | 124 | forward 2 | TM | N out |
| 199 | LG:407214.10:2000MAY19 | 203 | 289 | forward 2 | TM | N out |
| 200 | LG:204626.1:2000MAY19 | 19 | 99 | forward 1 | TM | N out |
| 202 | LI:476342.1:2000MAY01 | 39 | 122 | forward 3 | TM | N out |
| 203 | LI:1072759.1:2000MAY01 | 409 | 495 | forward 1 | TM | N in |
| 203 | LI:1072759.1:2000MAY01 | 889 | 951 | forward 1 | TM | N in |
| 203 | LI:1072759.1:2000MAY01 | 1387 | 1458 | forward 1 | TM | N in |
| 203 | LI:1072759.1:2000MAY01 | 1687 | 1770 | forward 1 | TM | N in |
| 203 | LI:1072759.1:2000MAY01 | 392 | 478 | forward 2 | TM | N out |
| 203 | LI:1072759.1:2000MAY01 | 1055 | 1132 | forward 2 | TM | N out |
| 203 | LI:1072759.1:2000MAY01 | 1424 | 1507 | forward 2 | TM | N out |
| 203 | LI:1072759.1:2000MAY01 | 1694 | 1768 | forward 2 | TM | N out |
| 203 | LI:1072759.1:2000MAY01 | 1191 | 1277 | forward 3 | TM | N out |
| 203 | LI:1072759.1:2000MAY01 | 1677 | 1760 | forward 3 | TM | N out |
| 204 | LG:998857.1:2000FEB18 | 1195 | 1281 | forward 1 | TM | N in |
| 204 | LG:998857.1:2000FEB18 | 164 | 226 | forward 2 | TM | N out |
| 204 | LG:998857.1:2000FEB18 | 344 | 400 | forward 2 | TM | N out |
| 204 | LG:998857.1:2000FEB18 | 398 | 460 | forward 2 | TM | N out |
| 204 | LG:998857.1:2000FEB18 | 1478 | 1561 | forward 2 | TM | N out |
| 205 | LG:482261.1:2000FEB18 | 19 | 93 | forward 1 | TM | N out |
| 205 | LG:482261.1:2000FEB18 | 890 | 961 | forward 2 | TM | N out |
| 205 | LG:482261.1:2000FEB18 | 1070 | 1123 | forward 2 | TM | N out |
| 205 | LG:482261.1:2000FEB18 | 21 | 89 | forward 3 | TM | N out |
| 205 | LG:482261.1:2000FEB18 | 1242 | 1292 | forward 3 | TM | N out |
| 206 | LG:480328.1:2000FEB18 | 436 | 522 | forward 1 | TM | N out |
| 206 | LG:480328.1:2000FEB18 | 568 | 642 | forward 1 | TM | N out |
| 206 | LG:480328.1:2000FEB18 | 769 | 849 | forward 1 | TM | N out |
| 206 | LG:480328.1:2000FEB18 | 967 | 1029 | forward 1 | TM | N out |
| 206 | LG:480328.1:2000FEB18 | 56 | 130 | forward 2 | TM | N in |
| 206 | LG:480328.1:2000FEB18 | 194 | 280 | forward 2 | TM | N in |
| 206 | LG:480328.1:2000FEB18 | 396 | 482 | forward 3 | TM | N out |
| 206 | LG:480328.1:2000FEB18 | 747 | 818 | forward 3 | TM | N out |
| 207 | LG:311197.1:2000MAY19 | 241 | 315 | forward 1 | TM | N in |
| 207 | LG:311197.1:2000MAY19 | 527 | 613 | forward 2 | TM | N out |
| 208 | LG:1054883.1:2000MAY19 | 76 | 129 | forward 1 | TM | N out |
| 208 | LG:1054883.1:2000MAY19 | 83 | 145 | forward 2 | TM | N out |
| 209 | LG:399395.1:2000MAY19 | 163 | 216 | forward 1 | TM | N out |
| 211 | LI:272913.22:2000MAY01 | 37 | 123 | forward 1 | TM | N in |

TABLE 4

| SEQ ID NO: | Component ID | Start | Stop |
|---|---|---|---|
| 1 | g1260446 | 2 | 316 |
| 1 | 6791379H1 | 1 | 397 |
| 1 | g1614819 | 215 | 655 |
| 1 | g1647514 | 244 | 543 |
| 2 | 5911492F8 | 1 | 467 |
| 2 | 5911492H1 | 1 | 271 |
| 2 | 5911492T8 | 303 | 633 |
| 3 | 5311056H1 | 591 | 753 |
| 3 | 6866213H1 | 784 | 1388 |
| 3 | 5659105H1 | 1261 | 1340 |
| 3 | 5498383R6 | 1291 | 1674 |
| 3 | g5553287 | 1 | 315 |
| 3 | 6989857H1 | 1 | 436 |
| 3 | 6955370H1 | 22 | 540 |
| 3 | g4534562 | 24 | 504 |
| 3 | g4390046 | 24 | 500 |
| 3 | g1192915 | 25 | 170 |
| 3 | g2003054 | 31 | 344 |
| 3 | 6818987J1 | 33 | 250 |
| 3 | 6818431J1 | 33 | 570 |
| 3 | g2003419 | 45 | 421 |
| 3 | g1551472 | 61 | 213 |
| 3 | 6147606H1 | 71 | 625 |
| 3 | 6990907H1 | 383 | 885 |
| 3 | 6866026H1 | 381 | 973 |
| 3 | 7067123H1 | 525 | 1069 |
| 3 | 5498383F6 | 573 | 1055 |
| 3 | 5498383H1 | 573 | 811 |
| 4 | 1709025H1 | 198 | 445 |
| 4 | 70319971D1 | 397 | 784 |
| 4 | 70320592D1 | 412 | 733 |
| 4 | 70320769D1 | 462 | 809 |
| 4 | 70317687D1 | 464 | 962 |
| 4 | 6483492H1 | 184 | 713 |
| 4 | 5006925H1 | 88 | 362 |
| 4 | 70513533D1 | 496 | 1069 |
| 4 | 70317606D1 | 522 | 961 |
| 4 | 1296898H1 | 262 | 495 |
| 4 | 6338333H1 | 640 | 1159 |
| 4 | g4332126 | 1 | 454 |
| 4 | 2659966F6 | 1 | 192 |
| 4 | 2659966T6 | 1 | 194 |
| 4 | 70513545V1 | 31 | 585 |
| 4 | 70514030D1 | 31 | 577 |
| 4 | 70514030V1 | 31 | 589 |
| 4 | 70513041V1 | 31 | 366 |
| 4 | 2659966H1 | 1 | 247 |
| 4 | 70512591V1 | 31 | 171 |
| 4 | 70512591D1 | 31 | 169 |
| 4 | 70318172D1 | 57 | 447 |
| 4 | 70513426V1 | 61 | 680 |
| 4 | 70320498D1 | 247 | 638 |
| 4 | 034207H1 | 1 | 290 |
| 4 | 70515532V1 | 24 | 612 |
| 4 | 70317785D1 | 295 | 738 |
| 4 | 70516168V1 | 24 | 391 |
| 4 | 70321122D1 | 370 | 783 |
| 4 | 70514482D1 | 24 | 278 |
| 4 | 70514482V1 | 24 | 278 |
| 4 | 70318245D1 | 25 | 395 |
| 4 | 70318040D1 | 29 | 409 |
| 4 | 70514959V1 | 1 | 300 |
| 5 | g2958900 | 175 | 294 |
| 5 | 167606H1 | 1 | 173 |
| 5 | 60123139D2 | 1 | 307 |
| 5 | 60123152D1 | 1 | 368 |
| 5 | 60123139D1 | 11 | 156 |
| 5 | 6127857H1 | 1 | 475 |
| 5 | g706800 | 1 | 340 |
| 5 | g690740 | 11 | 385 |
| 5 | 4454268H1 | 377 | 645 |
| 5 | 60123152B1 | 714 | 1379 |
| 5 | 1602161H1 | 603 | 798 |
| 5 | 6217784H1 | 857 | 1351 |
| 5 | 6217935H1 | 859 | 1356 |
| 5 | 1473544R6 | 214 | 653 |
| 5 | 1473544T1 | 249 | 653 |
| 5 | 1473544H1 | 408 | 653 |
| 5 | 60123139B1 | 948 | 1362 |
| 5 | 60124858B2 | 982 | 1362 |
| 5 | g4457589 | 222 | 601 |
| 5 | 1473544T6 | 247 | 569 |
| 5 | g715576 | 191 | 567 |
| 5 | g993694 | 255 | 560 |
| 5 | 2866203H1 | 459 | 557 |
| 5 | g682774 | 196 | 472 |
| 5 | g3213293 | 1 | 454 |
| 5 | g3148379 | 141 | 356 |
| 6 | 4835189H1 | 679 | 932 |
| 6 | g1920265 | 798 | 1061 |
| 6 | 4541025H1 | 804 | 1057 |
| 6 | 2330975H1 | 952 | 1172 |
| 6 | 2330975R6 | 952 | 1311 |
| 6 | 2430651T6 | 1141 | 1667 |
| 6 | 2571536T6 | 1144 | 1680 |
| 6 | 3878850T6 | 1244 | 1662 |
| 6 | g2930491 | 1302 | 1714 |
| 6 | g3844521 | 1455 | 1711 |
| 6 | 6623593H1 | 104 | 689 |
| 6 | 6623593J1 | 634 | 1236 |
| 6 | 4724029H1 | 1 | 133 |
| 6 | 5577111H1 | 14 | 200 |
| 6 | g2183982 | 84 | 445 |
| 6 | 3878850F6 | 119 | 524 |
| 6 | 3878850H1 | 120 | 399 |
| 6 | g2783388 | 239 | 721 |
| 6 | g2785249 | 239 | 710 |
| 6 | g1920471 | 284 | 439 |
| 6 | g1784782 | 388 | 828 |
| 6 | 6551102H1 | 429 | 951 |
| 6 | 6550902H1 | 429 | 1042 |
| 6 | 6551002H1 | 429 | 957 |
| 6 | g3645387 | 447 | 909 |
| 6 | 2430651H1 | 448 | 703 |
| 6 | 2430651R6 | 448 | 680 |
| 6 | 3712318H1 | 471 | 759 |
| 6 | g981405 | 485 | 787 |
| 6 | g1975879 | 1 | 219 |
| 6 | g1784993 | 585 | 909 |
| 6 | 1467511T7 | 1 | 438 |
| 6 | 6326238H1 | 8 | 306 |
| 6 | 526662H1 | 25 | 208 |
| 6 | 2856544H1 | 34 | 146 |
| 7 | 1980062R6 | 2 | 491 |
| 7 | 1980059H1 | 1 | 280 |
| 7 | 5573392H1 | 30 | 283 |
| 8 | 2642346H1 | 1 | 239 |
| 8 | 2662640H1 | 2 | 95 |
| 8 | 2523793H1 | 11 | 212 |
| 8 | 3155877H1 | 12 | 278 |
| 8 | 3539049H1 | 13 | 237 |
| 8 | 3179390H1 | 13 | 320 |
| 8 | 5543678H1 | 15 | 210 |
| 8 | 6834974H1 | 15 | 623 |
| 8 | 257055R6 | 16 | 423 |
| 8 | 133343H1 | 16 | 157 |
| 8 | 484198H1 | 16 | 163 |
| 8 | 257055H1 | 17 | 224 |
| 8 | 2346879H1 | 18 | 247 |
| 8 | 2346879F6 | 18 | 442 |
| 8 | g1985882 | 19 | 366 |
| 8 | 3156918H1 | 21 | 155 |
| 8 | 4934879H1 | 22 | 297 |
| 8 | g1751206 | 41 | 388 |
| 8 | 4024568H1 | 42 | 189 |
| 8 | 258140H1 | 58 | 433 |
| 8 | 2346879T6 | 167 | 442 |
| 8 | 2313878H1 | 229 | 495 |
| 8 | 6081177H1 | 264 | 720 |
| 8 | 3773491H1 | 306 | 476 |
| 8 | 6413047H1 | 401 | 935 |
| 8 | 4212712H1 | 407 | 675 |
| 8 | 6918705H1 | 413 | 837 |
| 8 | 3014942H1 | 416 | 684 |
| 8 | 1807684F6 | 483 | 905 |
| 8 | 1803962F6 | 483 | 841 |
| 8 | 1803962H1 | 483 | 760 |
| 8 | 1807684H1 | 483 | 746 |
| 8 | 3876768H1 | 529 | 819 |
| 8 | 6024789H1 | 553 | 695 |
| 8 | 4062033H1 | 555 | 779 |
| 8 | 4634711H1 | 615 | 877 |
| 8 | 3014303H1 | 667 | 964 |
| 8 | 3014303F6 | 667 | 1096 |
| 8 | 1633402H1 | 672 | 885 |
| 8 | 5865873H1 | 725 | 997 |
| 8 | 6552378H1 | 881 | 1430 |
| 8 | 2892794H1 | 907 | 1089 |
| 8 | 2892794F6 | 907 | 1389 |
| 8 | 6406936H1 | 935 | 1476 |
| 8 | 4902732H1 | 962 | 1225 |
| 8 | 1492959H1 | 970 | 1171 |
| 8 | g1686447 | 974 | 1299 |
| 8 | 5929258H1 | 974 | 1266 |
| 8 | 3509940H1 | 974 | 1230 |
| 8 | 4588644H1 | 983 | 1230 |
| 8 | 6979634H1 | 991 | 1412 |
| 8 | 946914H1 | 1010 | 1310 |
| 8 | g5854943 | 1083 | 1460 |
| 8 | 5544986H1 | 1095 | 1303 |
| 8 | 3860122H1 | 1133 | 1400 |
| 8 | g4260489 | 1228 | 1701 |
| 8 | 1724521H1 | 1385 | 1493 |
| 8 | 1724521F6 | 1385 | 1714 |
| 8 | 3026692H1 | 1429 | 1687 |
| 8 | 3881903H1 | 1460 | 1700 |
| 8 | 3014303T6 | 1504 | 1714 |
| 8 | 490059H1 | 1515 | 1763 |
| 8 | 1803962T6 | 1575 | 1714 |
| 8 | 692274H1 | 1583 | 1765 |
| 8 | 2313878T6 | 1587 | 1714 |
| 8 | g2436491 | 1623 | 1714 |
| 8 | g3277091 | 1654 | 1714 |
| 8 | 3319247H1 | 1718 | 1969 |
| 8 | 4917483H1 | 1916 | 2145 |
| 8 | 6826179J1 | 1971 | 2309 |
| 8 | 6826179H1 | 1971 | 2309 |
| 9 | 3088820H1 | 537 | 807 |
| 9 | 2012535H1 | 561 | 787 |
| 9 | 3232447H1 | 618 | 863 |
| 9 | 2536817H2 | 670 | 926 |
| 9 | g2209764 | 832 | 1178 |
| 9 | 7260050H1 | 1 | 537 |
| 9 | 2457623F6 | 101 | 601 |
| 9 | 6987326H1 | 110 | 654 |
| 9 | 7032756H1 | 151 | 720 |
| 9 | 6457158H1 | 350 | 878 |
| 9 | 2890632F6 | 351 | 531 |
| 9 | 2893095F6 | 351 | 909 |
| 9 | 2893095H1 | 351 | 614 |
| 9 | 7166124H1 | 401 | 930 |
| 9 | 4760777H1 | 427 | 714 |
| 9 | 3160745H1 | 437 | 710 |
| 9 | g2006716 | 479 | 776 |
| 9 | g2209659 | 481 | 940 |
| 9 | 3362481H1 | 514 | 763 |
| 9 | 1751755H1 | 520 | 735 |
| 9 | 6141843H1 | 536 | 793 |
| 10 | 6584158H1 | 14 | 592 |
| 10 | 3401851H1 | 4 | 241 |
| 10 | 3403550H1 | 1 | 259 |
| 10 | 3404251H1 | 3 | 258 |
| 10 | g1137215 | 2430 | 2639 |
| 10 | 3378612H1 | 2451 | 2638 |
| 10 | 6584049H1 | 6 | 562 |
| 10 | 3402034H1 | 10 | 229 |
| 10 | g307503 | 16 | 2638 |
| 10 | 70779637V1 | 2077 | 2632 |
| 10 | 7258175H1 | 2121 | 2603 |
| 10 | 3961980H1 | 2130 | 2252 |
| 10 | 6584189T1 | 2152 | 2553 |
| 10 | 6584158T1 | 2159 | 2659 |
| 10 | g3785069 | 2394 | 2641 |
| 10 | 981592T6 | 2170 | 2701 |
| 10 | 3385383T6 | 2171 | 2601 |
| 10 | 1221889T6 | 2202 | 2700 |
| 10 | 70779908V1 | 2348 | 2654 |
| 10 | 70776665V1 | 1884 | 2562 |
| 10 | 70775642V1 | 1981 | 2519 |
| 10 | 70776673V1 | 2022 | 2640 |
| 10 | 6584049T1 | 2035 | 2569 |
| 10 | 3401765H1 | 2050 | 2287 |
| 10 | 3401379H1 | 15 | 260 |
| 10 | 3404170H1 | 18 | 267 |
| 11 | 6905943H1 | 1 | 534 |
| 12 | 70562343V1 | 1 | 628 |
| 12 | 70560334V1 | 1 | 538 |
| 12 | 70559056V1 | 2 | 275 |
| 12 | 70560532V1 | 12 | 269 |
| 12 | 70559641V1 | 12 | 383 |
| 12 | 70559213V1 | 12 | 511 |
| 12 | 70561550V1 | 12 | 575 |
| 12 | 70561738V1 | 12 | 582 |
| 12 | 70559422V1 | 13 | 631 |
| 12 | 70559480V1 | 12 | 687 |
| 12 | 758040H1 | 12 | 272 |
| 12 | 70559415V1 | 13 | 605 |
| 12 | 758040R6 | 12 | 310 |
| 12 | 70561876V1 | 13 | 585 |
| 12 | 70559130V1 | 35 | 384 |
| 12 | g2576304 | 119 | 2175 |
| 12 | 70561182V1 | 150 | 812 |
| 12 | 70560479V1 | 217 | 834 |
| 12 | 70557043V1 | 221 | 595 |
| 12 | 70562198V1 | 227 | 819 |
| 12 | 70562312V1 | 245 | 894 |
| 12 | 70561005V1 | 249 | 895 |
| 12 | 70559925V1 | 254 | 840 |
| 12 | 70556220V1 | 274 | 841 |
| 12 | 70561793V1 | 282 | 939 |
| 12 | 70559002V1 | 293 | 888 |
| 12 | 6127888H1 | 351 | 824 |
| 12 | 70557039V1 | 359 | 611 |
| 12 | 70559636V1 | 391 | 1033 |
| 12 | 70561656V1 | 409 | 852 |
| 12 | 70560742V1 | 507 | 990 |
| 12 | 70561641V1 | 594 | 1066 |
| 12 | g873095 | 657 | 1032 |
| 12 | 70449484V1 | 674 | 894 |
| 12 | 6848717H1 | 732 | 1253 |
| 12 | 70558944V1 | 1495 | 2173 |
| 12 | 70450051V1 | 1550 | 1731 |
| 12 | 70561063V1 | 1607 | 2025 |
| 12 | g5361507 | 1719 | 2172 |
| 12 | g4735148 | 1778 | 2172 |
| 12 | g4875543 | 1779 | 2195 |
| 12 | 758040T6 | 1784 | 2147 |
| 12 | g872996 | 1824 | 2188 |
| 12 | g3840560 | 1830 | 2175 |
| 12 | g4333943 | 2114 | 2199 |
| 13 | 2734453F6 | 1 | 435 |
| 13 | 2734453H1 | 1 | 252 |
| 14 | 4860612T7 | 1184 | 1574 |
| 14 | g902319 | 1020 | 1492 |
| 14 | g778351 | 1137 | 1386 |
| 14 | g1638619 | 1140 | 1268 |
| 14 | 6929341H1 | 724 | 1222 |
| 14 | 1675892T6 | 754 | 1203 |
| 14 | 1720010F6 | 696 | 1079 |
| 14 | 1720010H1 | 696 | 922 |
| 14 | 4711951H1 | 675 | 833 |
| 14 | 5496406H1 | 546 | 795 |
| 14 | 6420632H1 | 79 | 637 |
| 14 | 2195346F6 | 1 | 450 |
| 14 | 2195346H1 | 344 | 450 |
| 14 | 3933140F6 | 11 | 351 |
| 14 | 3933140H1 | 96 | 351 |
| 14 | 6020638H1 | 1 | 263 |
| 14 | g5880310 | 52 | 255 |
| 14 | g2156573 | 11 | 254 |
| 14 | g2264198 | 30 | 253 |
| 14 | 6936789H1 | 11 | 173 |
| 14 | 5043956H1 | 11 | 159 |
| 15 | 1897573H1 | 1 | 259 |
| 15 | 4337619T6 | 137 | 645 |
| 15 | 5436383H1 | 598 | 849 |
| 16 | 1671029F6 | 1 | 443 |
| 16 | 1671029H1 | 72 | 144 |
| 16 | 1671029T6 | 388 | 443 |
| 16 | g2238932 | 388 | 443 |
| 16 | g2243675 | 388 | 443 |
| 17 | g2219785 | 236 | 531 |
| 17 | 5099781H1 | 378 | 604 |
| 17 | 3254347R6 | 527 | 1132 |
| 17 | 5668261H1 | 661 | 889 |
| 17 | 4289028H1 | 781 | 1061 |
| 17 | g3931955 | 822 | 1267 |
| 17 | g3401348 | 823 | 1271 |
| 17 | g2825326 | 825 | 1144 |
| 17 | 2750359R6 | 824 | 1242 |
| 17 | 2750359T6 | 847 | 1241 |
| 17 | 2351445H1 | 151 | 367 |
| 17 | 2750359H1 | 968 | 1241 |
| 17 | 2203345H1 | 1018 | 1169 |
| 17 | 3254347H1 | 979 | 1089 |
| 17 | 4822216H1 | 1202 | 1393 |
| 17 | 3184753F6 | 1 | 472 |
| 17 | 5108214T6 | 60 | 258 |
| 17 | 3184753T6 | 60 | 312 |
| 17 | g4287070 | 152 | 617 |
| 17 | g2524411 | 160 | 493 |
| 17 | 3184753H1 | 228 | 472 |
| 17 | g2788109 | 236 | 513 |
| 18 | 6829315H1 | 314 | 884 |
| 18 | g3109791 | 492 | 811 |
| 18 | g5452473 | 492 | 650 |
| 18 | g4564783 | 26 | 423 |
| 18 | g5438746 | 1 | 423 |
| 18 | g3805312 | 34 | 423 |
| 18 | g4372490 | 78 | 423 |
| 18 | g2954208 | 76 | 423 |
| 18 | g2954218 | 142 | 422 |
| 18 | g3307490 | 142 | 344 |
| 18 | 2011384H1 | 190 | 263 |
| 19 | 3106785H1 | 747 | 1023 |
| 19 | g565430 | 783 | 1150 |
| 19 | g1505888 | 812 | 1132 |
| 19 | g892901 | 859 | 1159 |
| 19 | g892900 | 1 | 179 |
| 19 | 3436153H1 | 1 | 269 |
| 19 | g4737696 | 1 | 452 |
| 19 | 839090H1 | 73 | 301 |
| 19 | 6938671H1 | 206 | 685 |
| 19 | 6939071H1 | 207 | 731 |
| 19 | g646973 | 427 | 675 |
| 19 | g1277657 | 484 | 934 |
| 19 | 2755039H1 | 596 | 767 |
| 19 | 838332T6 | 615 | 1116 |
| 19 | 4800144H1 | 651 | 916 |
| 19 | 2084151H1 | 664 | 945 |
| 20 | 6199407H1 | 1 | 558 |
| 20 | 2519336F6 | 37 | 448 |
| 20 | 2519336H1 | 37 | 198 |
| 20 | 6793417H1 | 61 | 331 |
| 20 | 1639314H1 | 71 | 248 |
| 20 | 6818283H1 | 75 | 652 |
| 20 | 1984661R6 | 94 | 490 |
| 20 | 1984662H1 | 94 | 368 |
| 20 | 1984661T6 | 127 | 543 |
| 20 | 4021069H1 | 177 | 388 |
| 20 | 3456613H1 | 177 | 427 |
| 20 | 4923878H1 | 211 | 494 |
| 20 | 6847871H1 | 274 | 451 |
| 20 | g5151844 | 373 | 585 |
| 21 | g4281322 | 2017 | 2463 |
| 21 | 3934096H1 | 2040 | 2211 |
| 21 | 3934274H1 | 2042 | 2187 |
| 21 | 1648485T6 | 2045 | 2159 |
| 21 | 1648242H1 | 2052 | 2198 |
| 21 | 1648485H1 | 2052 | 2198 |
| 21 | 1648485F6 | 2052 | 2198 |
| 21 | 4676772H1 | 2056 | 2317 |
| 21 | 1004843T6 | 2092 | 2693 |
| 21 | g4533314 | 2101 | 2207 |
| 21 | 3416826H1 | 2134 | 2365 |
| 21 | 5304471H1 | 2184 | 2431 |
| 21 | 1310089F6 | 2250 | 2718 |
| 21 | 1310089H1 | 2250 | 2448 |
| 21 | 2461918H1 | 2266 | 2461 |
| 21 | g4330296 | 2658 | 2953 |
| 21 | g3178479 | 2713 | 2950 |
| 21 | g856243 | 2748 | 3020 |
| 21 | 3951063F6 | 1 | 417 |
| 21 | 3951063H1 | 1 | 285 |
| 21 | g2114724 | 215 | 609 |
| 21 | 1804812F6 | 311 | 871 |
| 21 | 1804812H1 | 311 | 582 |
| 21 | 1804772H1 | 311 | 530 |
| 21 | 2662164H1 | 346 | 588 |
| 21 | 2011927H1 | 365 | 608 |
| 21 | 4863613H1 | 380 | 645 |
| 21 | 3934325H1 | 386 | 672 |
| 21 | 3763239H1 | 402 | 671 |
| 21 | 1004843R6 | 502 | 864 |
| 21 | 1004843H1 | 502 | 706 |
| 21 | g846375 | 547 | 889 |
| 21 | 5810205H1 | 585 | 885 |
| 21 | 384851H1 | 652 | 926 |
| 21 | g1861137 | 688 | 1180 |
| 21 | 2670050H1 | 722 | 974 |
| 21 | 4118973H1 | 762 | 1013 |
| 21 | 2744119H1 | 784 | 968 |
| 21 | g1081212 | 787 | 1107 |
| 21 | 5576610H1 | 879 | 1134 |
| 21 | 6418368H1 | 926 | 1226 |
| 21 | 5864111H1 | 950 | 1233 |
| 21 | 5033538H1 | 997 | 1264 |
| 21 | 3511167H1 | 1010 | 1288 |
| 21 | 5805062H1 | 1021 | 1225 |
| 21 | 4873652H1 | 1038 | 1293 |
| 21 | 6614566H1 | 1064 | 1565 |
| 21 | 2645969F6 | 1085 | 1588 |
| 21 | 2645969H1 | 1085 | 1330 |
| 21 | g2112831 | 1112 | 1336 |
| 21 | 3742279H1 | 1144 | 1422 |
| 21 | 6334985H1 | 1249 | 1754 |
| 21 | 5292787H2 | 1287 | 1357 |
| 21 | g853299 | 1363 | 1679 |
| 21 | 5742830H1 | 1399 | 1713 |
| 21 | 777713H1 | 1400 | 1625 |
| 21 | 777713R6 | 1401 | 1702 |
| 21 | 2291319H1 | 1424 | 1651 |
| 21 | 3241291H1 | 1436 | 1673 |
| 21 | 1832630H1 | 1481 | 1750 |
| 21 | 1804812T6 | 1571 | 2172 |
| 21 | 1481768H1 | 1572 | 1793 |
| 21 | 3730896H1 | 1583 | 1874 |
| 21 | 4442950H1 | 1631 | 1773 |
| 21 | 2794894H1 | 1648 | 1896 |
| 21 | 4161422H1 | 1664 | 1913 |
| 21 | 4163675H1 | 1665 | 1779 |
| 21 | g2787436 | 1672 | 1875 |
| 21 | 1898917T6 | 1693 | 2159 |
| 21 | 5026603H1 | 1695 | 1945 |
| 21 | 3726809H1 | 1753 | 2047 |
| 21 | 1891483T6 | 1803 | 2160 |
| 21 | 2293309H1 | 1836 | 2077 |
| 21 | 1214225H1 | 1850 | 2020 |
| 21 | g856337 | 1855 | 2128 |
| 21 | g3733909 | 1913 | 2198 |
| 21 | g3890798 | 1917 | 2386 |
| 21 | 5696574H1 | 1925 | 2048 |
| 21 | g1081162 | 1942 | 2201 |
| 21 | 2645969T6 | 1964 | 2222 |
| 21 | g1860963 | 1970 | 2387 |
| 21 | 777713T6 | 1983 | 2420 |
| 21 | g4149122 | 1984 | 2203 |
| 21 | 4875545H1 | 1994 | 2292 |
| 22 | 487652H1 | 1 | 242 |
| 22 | 487652R6 | 1 | 232 |
| 22 | g2003010 | 47 | 328 |
| 22 | 6442754H1 | 49 | 581 |
| 22 | 6152037H1 | 50 | 234 |
| 22 | 7072795H1 | 75 | 624 |
| 22 | 3126080H1 | 75 | 234 |
| 22 | 491193H1 | 83 | 234 |
| 22 | 4751976H1 | 90 | 232 |
| 22 | 7100707H1 | 100 | 609 |
| 22 | 2292643H1 | 102 | 232 |
| 22 | 6140970H1 | 105 | 232 |
| 22 | 4066069H1 | 110 | 234 |
| 22 | 5007390H1 | 113 | 231 |
| 22 | 4773161H1 | 130 | 415 |
| 22 | 6993149H1 | 145 | 680 |
| 22 | 572575H1 | 150 | 232 |
| 22 | 5756677H1 | 180 | 483 |
| 22 | 6064882H1 | 346 | 644 |
| 22 | 5372689H1 | 367 | 514 |
| 22 | 3123257H1 | 367 | 493 |
| 22 | g2022792 | 381 | 648 |
| 22 | 5068149H1 | 503 | 782 |
| 22 | 5894157H1 | 560 | 759 |
| 22 | 5898812H1 | 560 | 853 |
| 22 | 2621801H1 | 561 | 824 |
| 22 | 6401738H1 | 574 | 844 |
| 22 | 2122453H1 | 622 | 902 |
| 22 | 2122453F6 | 622 | 764 |
| 22 | 5681165H1 | 629 | 889 |
| 22 | 5277977H1 | 637 | 892 |
| 22 | 4645753H1 | 659 | 921 |
| 22 | 6193452H1 | 705 | 1000 |
| 22 | 6193441H1 | 705 | 963 |
| 22 | 746066H1 | 713 | 961 |
| 22 | 4771093H1 | 754 | 1022 |
| 22 | g2054100 | 771 | 1123 |
| 22 | g4762720 | 796 | 1252 |
| 22 | g2321843 | 801 | 1217 |
| 22 | g3017276 | 804 | 1256 |
| 22 | g483457 | 850 | 1222 |
| 22 | g4762705 | 865 | 1252 |
| 22 | 4338534H1 | 870 | 1155 |
| 22 | 628211H1 | 887 | 1051 |
| 22 | 5563275H1 | 922 | 1144 |
| 22 | 5286207H1 | 952 | 1210 |
| 22 | 6160626H1 | 953 | 1244 |
| 22 | 6838871H1 | 1013 | 1491 |
| 22 | 2400686H1 | 1068 | 1153 |
| 22 | g866503 | 1107 | 1440 |
| 22 | 6747627H1 | 1122 | 1593 |
| 22 | g920433 | 1143 | 1481 |
| 22 | 2294209R6 | 1179 | 1688 |
| 22 | 2294209H1 | 1179 | 1444 |
| 22 | g1471741 | 1213 | 1600 |
| 22 | g1332314 | 1213 | 1712 |
| 22 | 6021757H1 | 1214 | 1822 |
| 22 | 2189963H1 | 1229 | 1505 |
| 22 | g5766946 | 1310 | 1760 |
| 22 | g2524669 | 1309 | 1776 |
| 22 | g2178309 | 1317 | 1448 |
| 22 | g4762067 | 1326 | 1779 |
| 22 | g5361693 | 1328 | 1778 |
| 22 | g4074013 | 1332 | 1773 |
| 22 | g5849550 | 1331 | 1762 |
| 22 | 6736883H1 | 1355 | 1730 |
| 22 | g2183535 | 1356 | 1766 |
| 22 | g4332913 | 1357 | 1772 |
| 22 | g3147585 | 1357 | 1758 |
| 22 | g2053942 | 1361 | 1759 |
| 22 | g1332315 | 1371 | 1839 |
| 22 | 3918780H1 | 1373 | 1665 |
| 22 | g3884754 | 1379 | 1765 |
| 22 | 5207656H1 | 1382 | 1616 |
| 22 | 2292948H1 | 1388 | 1552 |
| 22 | 1373715H1 | 1388 | 1603 |
| 22 | 4742391H1 | 1388 | 1647 |
| 22 | 2749392H1 | 1400 | 1627 |
| 22 | g1241849 | 1408 | 1559 |
| 22 | g6046888 | 1408 | 1785 |
| 22 | 2294209T6 | 1428 | 1945 |
| 22 | g1471742 | 1441 | 1766 |
| 22 | g4332659 | 1471 | 1772 |
| 22 | g3842415 | 1473 | 1785 |
| 22 | g2841585 | 1474 | 1756 |
| 22 | g2003011 | 1479 | 1772 |
| 22 | 2501320H1 | 1513 | 1738 |
| 22 | g3961438 | 1525 | 1988 |
| 22 | g2022791 | 1559 | 1757 |
| 22 | g6132921 | 1559 | 1977 |
| 22 | 5690247H1 | 1568 | 1843 |
| 22 | g1496515 | 1580 | 1977 |
| 22 | g2252260 | 1620 | 1984 |
| 22 | g3739177 | 1650 | 1831 |
| 22 | 5338636H1 | 1671 | 1915 |
| 22 | 5710854H2 | 1686 | 2004 |
| 22 | 3223930H1 | 1686 | 1772 |
| 22 | 4255692H1 | 1701 | 1756 |
| 22 | g1210253 | 1725 | 1980 |
| 22 | g920434 | 1730 | 1946 |
| 22 | g1210208 | 1763 | 1980 |
| 22 | 4320678H1 | 1841 | 1973 |
| 22 | g884068 | 1924 | 2233 |
| 23 | 6991066H1 | 1 | 190 |
| 23 | 4753733H1 | 121 | 279 |
| 23 | 4753733F8 | 122 | 613 |
| 23 | 7065882H1 | 183 | 679 |
| 23 | g5676063 | 251 | 722 |
| 23 | 1576986F6 | 270 | 661 |
| 23 | 1576986H1 | 270 | 349 |
| 23 | 1576986T6 | 275 | 683 |
| 23 | g3843718 | 364 | 724 |
| 23 | g3750491 | 408 | 722 |
| 23 | 3604635H1 | 409 | 584 |
| 23 | g793096 | 600 | 881 |
| 24 | 6756043H1 | 101 | 504 |
| 24 | 3845553H1 | 368 | 574 |
| 24 | 5057288F9 | 382 | 1021 |
| 24 | 5057288H1 | 382 | 660 |
| 24 | 5065461F8 | 404 | 839 |
| 24 | 2359950H1 | 625 | 748 |
| 24 | 6112349H1 | 792 | 1081 |
| 24 | 5444375H1 | 912 | 1131 |
| 24 | g4264503 | 989 | 1390 |
| 24 | 3385663T6 | 1011 | 1248 |
| 24 | 3845553T6 | 1009 | 1248 |
| 24 | 3385663F6 | 1 | 315 |
| 24 | 3385663H1 | 1 | 257 |
| 24 | 5468404H1 | 3 | 271 |
| 24 | 5468404F8 | 3 | 587 |
| 24 | 5072501H1 | 11 | 292 |
| 24 | 6917202H1 | 10 | 259 |
| 24 | 4148204T6 | 1176 | 1391 |
| 24 | 4148204H1 | 1183 | 1435 |
| 24 | 4148204F6 | 1183 | 1390 |
| 24 | g4532148 | 1256 | 1361 |
| 24 | 213841R6 | 1325 | 1390 |
| 24 | 213841T6 | 1325 | 1393 |
| 24 | 213841H1 | 1325 | 1390 |
| 24 | 213833H1 | 1325 | 1390 |
| 25 | 7204172H1 | 1 | 536 |
| 25 | 6819816H1 | 368 | 566 |
| 25 | 6836688H1 | 383 | 960 |
| 25 | 6349781H2 | 434 | 616 |
| 25 | 7280863H1 | 477 | 880 |
| 25 | g4081510 | 513 | 860 |
| 25 | 6862186H1 | 519 | 806 |
| 25 | g5543132 | 541 | 939 |
| 25 | g4196555 | 541 | 821 |
| 25 | g5636120 | 541 | 982 |
| 25 | g3246528 | 541 | 923 |
| 25 | g5636142 | 541 | 1018 |
| 25 | g2568732 | 577 | 1024 |
| 25 | 3747228H1 | 589 | 887 |
| 25 | 6600395H1 | 587 | 999 |
| 25 | 6978931H1 | 605 | 954 |
| 25 | 2240520H1 | 629 | 730 |
| 25 | 2240520F6 | 641 | 967 |
| 25 | 5626829H1 | 663 | 931 |
| 25 | g2162199 | 663 | 863 |
| 25 | 6862047H1 | 699 | 978 |
| 25 | 7281807H1 | 809 | 1041 |
| 25 | g2209650 | 815 | 1308 |
| 25 | 6216076H1 | 841 | 1322 |
| 25 | 7238641H1 | 862 | 978 |
| 25 | 6210267H1 | 868 | 970 |
| 25 | 7039186H1 | 940 | 1508 |
| 25 | g2209760 | 1092 | 1569 |
| 26 | 7281748H1 | 1 | 439 |
| 26 | 7281705H1 | 1 | 574 |
| 27 | 925628R6 | 1574 | 1961 |
| 27 | 6466550H1 | 228 | 746 |
| 27 | 035057H1 | 483 | 638 |
| 27 | 2173285F6 | 571 | 968 |
| 27 | 2173285H1 | 571 | 762 |
| 27 | 1743077R6 | 577 | 1069 |
| 27 | 1743077H1 | 577 | 892 |
| 27 | 3344276H1 | 598 | 856 |
| 27 | 6579246H1 | 697 | 1025 |
| 27 | 5637939F8 | 825 | 1101 |
| 27 | 5637939H1 | 825 | 1082 |
| 27 | 5624530R8 | 836 | 1190 |
| 27 | 5637939R8 | 1016 | 1380 |
| 27 | 3483518H1 | 1163 | 1470 |
| 27 | 6817649J1 | 1402 | 1969 |
| 27 | 2966432H1 | 1421 | 1627 |
| 27 | g3419012 | 1500 | 1961 |
| 27 | g2178116 | 1505 | 1630 |
| 27 | g2715860 | 1512 | 1962 |
| 27 | 3891858T6 | 1559 | 1901 |
| 27 | 3891858F6 | 1566 | 1961 |
| 27 | 3891858H1 | 1566 | 1796 |
| 27 | 925628T6 | 1574 | 1943 |
| 27 | 925628H1 | 1574 | 1797 |
| 27 | g5113339 | 1766 | 2184 |
| 27 | 7286802H1 | 1 | 500 |
| 27 | 3490274H1 | 26 | 319 |
| 27 | 6817649H1 | 55 | 308 |
| 27 | 6779822H1 | 68 | 617 |
| 27 | 1343838H1 | 166 | 433 |
| 28 | 6827746J1 | 1 | 328 |
| 28 | g4372436 | 2 | 487 |
| 28 | 7277307H1 | 1 | 320 |
| 28 | g1109781 | 68 | 2457 |
| 28 | g2987914 | 238 | 414 |
| 28 | 3242379H1 | 460 | 594 |
| 28 | 5843682H1 | 675 | 814 |
| 28 | 6244385H1 | 750 | 960 |
| 28 | 3560794H1 | 878 | 978 |
| 28 | 6146228H1 | 1389 | 1559 |
| 28 | g5454470 | 1759 | 2219 |
| 28 | g5634013 | 1818 | 2245 |
| 28 | g3933775 | 1835 | 2188 |
| 28 | 6824121H1 | 1864 | 2239 |
| 28 | 6824121J1 | 1864 | 2239 |
| 28 | 6789580H1 | 1898 | 2361 |
| 28 | g5596185 | 1915 | 2221 |
| 28 | g1137662 | 1994 | 2365 |
| 28 | g3917233 | 2020 | 2409 |
| 28 | 3222719H1 | 2019 | 2299 |
| 28 | g4487317 | 2029 | 2459 |
| 28 | g2878126 | 2045 | 2386 |
| 28 | 925319R6 | 2061 | 2458 |
| 28 | 925319T6 | 2061 | 2419 |
| 28 | 925319H1 | 2061 | 2370 |
| 28 | g6463796 | 2103 | 2467 |
| 28 | 2175458H1 | 2139 | 2397 |
| 28 | g1153903 | 2165 | 2458 |
| 28 | g1114183 | 2165 | 2444 |
| 28 | g3844190 | 2203 | 2458 |
| 28 | 6193937H1 | 2265 | 2534 |
| 28 | g5364673 | 2276 | 2476 |
| 29 | 6450124H1 | 992 | 1374 |
| 29 | 70254601V1 | 1036 | 1539 |
| 29 | 70250522V1 | 1050 | 1299 |
| 29 | 70255407V1 | 1073 | 1570 |
| 29 | g2004277 | 1088 | 1409 |
| 29 | 70247748V1 | 1116 | 1378 |
| 29 | 70249044V1 | 1117 | 1269 |
| 29 | 70256246V1 | 1130 | 1541 |
| 29 | 70248757V1 | 1132 | 1311 |
| 29 | 7331424H1 | 1149 | 1638 |
| 29 | 70248822V1 | 1157 | 1333 |
| 29 | 70656623V1 | 1723 | 2219 |
| 29 | 661089H1 | 1723 | 1986 |
| 29 | 661089R6 | 1723 | 2224 |
| 29 | 6444789H1 | 1742 | 2329 |
| 29 | 70248827V1 | 1769 | 2103 |
| 29 | 6551207H1 | 1771 | 2379 |
| 29 | 6745795H1 | 1775 | 1933 |
| 29 | 6977014H1 | 1787 | 2347 |
| 29 | 70258294V1 | 1791 | 2007 |
| 29 | 70254633V1 | 1805 | 2279 |
| 29 | 70255157V1 | 1838 | 2268 |
| 29 | 6746363H1 | 1854 | 2506 |
| 29 | 70655542V1 | 1865 | 1948 |
| 29 | 6561163H1 | 1872 | 2335 |
| 29 | 7239061H1 | 1879 | 2417 |
| 29 | 70247905V1 | 1879 | 2117 |
| 29 | 70255827V1 | 764 | 1239 |
| 29 | 70255250V1 | 842 | 1401 |
| 29 | 70248754V1 | 866 | 1233 |
| 29 | 70256314V1 | 917 | 1512 |
| 29 | 70254664V1 | 444 | 930 |
| 29 | 70254648V1 | 463 | 958 |
| 29 | 70257273V1 | 531 | 636 |
| 29 | g3778732 | 559 | 1002 |
| 29 | 4023624H1 | 1 | 286 |
| 29 | 70249613V1 | 1 | 198 |
| 29 | 3269702H1 | 8 | 254 |
| 29 | 70249634V1 | 142 | 374 |
| 29 | 1286494H1 | 165 | 402 |
| 29 | 7191139H2 | 175 | 755 |
| 29 | 5841654H1 | 175 | 446 |
| 29 | 7120789H1 | 398 | 702 |
| 29 | 70255670V1 | 413 | 949 |
| 29 | 70255767V1 | 1672 | 2119 |
| 29 | 3272240F6 | 1165 | 1617 |
| 29 | 3272240H1 | 1166 | 1413 |
| 29 | 70257558V1 | 1171 | 1601 |
| 29 | 70248599V1 | 1202 | 1420 |
| 29 | 70250795V1 | 1247 | 1524 |
| 29 | 70249059V1 | 1304 | 1679 |
| 29 | 70250085V1 | 1310 | 1485 |
| 29 | 70251290V1 | 1314 | 1573 |
| 29 | 70255060V1 | 1326 | 1720 |
| 29 | 70254762V1 | 1330 | 1860 |
| 29 | 70257824V1 | 1339 | 1525 |
| 29 | 70255199V1 | 1341 | 1753 |
| 29 | 70250692V1 | 1345 | 1576 |
| 29 | 70257468V1 | 1347 | 1462 |
| 29 | 70251556V1 | 1351 | 1435 |
| 29 | 70255729V1 | 1361 | 1869 |
| 29 | 70255867V1 | 1392 | 1903 |
| 29 | 70255422V1 | 1366 | 1870 |
| 29 | 70254725V1 | 1690 | 2218 |
| 29 | 70254417V1 | 1671 | 1916 |
| 29 | 7033280H1 | 1691 | 2365 |
| 29 | 1741694R6 | 1410 | 1866 |
| 29 | 1741694H1 | 1410 | 1587 |
| 29 | 70256209V1 | 1414 | 1915 |
| 29 | 7079231H1 | 1418 | 1892 |
| 29 | 70248162V1 | 1486 | 1604 |
| 29 | 70254647V1 | 612 | 1111 |
| 29 | 7077379H1 | 627 | 1163 |
| 29 | 7237267H1 | 1907 | 2197 |
| 29 | 70660270V1 | 1926 | 2390 |
| 29 | 70255193V1 | 1976 | 2586 |
| 29 | 70656301V1 | 1979 | 2231 |
| 29 | 71273576V1 | 1979 | 2197 |
| 29 | 6620779H1 | 2006 | 2603 |
| 29 | 70655675V1 | 2023 | 2430 |
| 29 | 70255204V1 | 2038 | 2632 |
| 29 | 1741694T6 | 2052 | 2625 |
| 29 | 6121206F8 | 2130 | 2669 |
| 29 | 5267406H1 | 2070 | 2344 |
| 29 | 6120592H1 | 2110 | 2682 |
| 29 | 6128080H1 | 2110 | 2689 |
| 29 | 6120292H1 | 2110 | 2671 |
| 29 | 6127177H1 | 2110 | 2671 |
| 29 | 3269702T6 | 2111 | 2623 |
| 29 | 70254909V1 | 2152 | 2688 |
| 29 | 4001246R6 | 2158 | 2517 |
| 29 | 4001246H1 | 2158 | 2458 |
| 29 | 70659041V1 | 2163 | 2394 |
| 29 | g5864796 | 2171 | 2685 |
| 29 | g2913171 | 2184 | 2622 |
| 29 | 6752303H1 | 2193 | 2671 |
| 29 | 70658326V1 | 2194 | 2559 |
| 29 | 70256172V1 | 2209 | 2671 |
| 29 | 2287809R6 | 2232 | 2681 |
| 29 | 2287809T6 | 2232 | 2629 |
| 29 | 2287809H1 | 2232 | 2458 |
| 29 | 6834934H1 | 2233 | 2671 |
| 29 | 2255790H1 | 2260 | 2543 |
| 29 | 70657495V1 | 2264 | 2391 |
| 29 | 6835034H1 | 2288 | 2570 |
| 29 | 70254456V1 | 2316 | 2671 |
| 29 | 661089T6 | 2355 | 2639 |
| 29 | 70249168V1 | 2452 | 2682 |
| 29 | 6059187H1 | 2608 | 2671 |
| 29 | 6059196H1 | 2606 | 2682 |
| 29 | 70255727V1 | 977 | 1493 |
| 29 | 70257455V1 | 1692 | 1951 |
| 29 | 70257816V1 | 1397 | 1604 |
| 29 | 70257088V2 | 1659 | 1980 |
| 29 | 70255058V1 | 1663 | 2172 |
| 29 | 5779331H1 | 1663 | 1928 |
| 29 | 70248161V1 | 1487 | 1604 |
| 29 | 6536779H1 | 1490 | 2108 |
| 29 | 70254435V1 | 1509 | 2113 |
| 29 | 70254449V1 | 1538 | 1906 |
| 29 | 70257853V1 | 1570 | 1900 |
| 29 | 70254760V1 | 1571 | 1970 |
| 29 | 70255434V1 | 1577 | 2080 |
| 29 | 5191169H1 | 1609 | 1875 |
| 29 | 70257088V1 | 1659 | 1980 |
| 29 | 70255260V1 | 1694 | 2245 |
| 29 | 70255174V1 | 1695 | 2113 |
| 29 | g756281 | 1698 | 2066 |
| 29 | 70247381V1 | 1704 | 2145 |
| 29 | 70659718V1 | 1723 | 2028 |
| 29 | 70254418V1 | 675 | 1188 |
| 29 | 70258031V1 | 699 | 905 |
| 29 | 7178470H1 | 651 | 1108 |
| 29 | 70256308V1 | 672 | 1122 |
| 30 | 70780965V1 | 4 | 464 |
| 30 | 2359950H1 | 634 | 758 |
| 30 | 70777278V1 | 648 | 1196 |
| 30 | 70780989V1 | 653 | 1111 |
| 30 | 70779952V1 | 697 | 1217 |
| 30 | 70777355V1 | 715 | 1195 |
| 30 | 70771142V1 | 716 | 1207 |
| 30 | 70775784V1 | 751 | 1132 |
| 30 | 70769785V1 | 764 | 1127 |
| 30 | 6112349H1 | 809 | 1108 |
| 30 | 70776223V1 | 839 | 1239 |
| 30 | 5444375H1 | 933 | 1164 |
| 30 | 5057288T8 | 997 | 1251 |
| 30 | g4264503 | 1011 | 1388 |
| 30 | 3385663T6 | 1033 | 1284 |
| 30 | 3845553T6 | 1031 | 1284 |
| 30 | 70776533V1 | 1086 | 1284 |
| 30 | 70776877V1 | 1087 | 1284 |
| 30 | 4148204T6 | 1174 | 1389 |
| 30 | 4148204F6 | 1181 | 1388 |
| 30 | 4148204H1 | 1181 | 1433 |
| 30 | g4532148 | 1254 | 1359 |
| 30 | 5072501H1 | 11 | 296 |
| 30 | 70770035V1 | 207 | 641 |
| 30 | 70769523V1 | 245 | 581 |
| 30 | 70779544V1 | 337 | 548 |
| 30 | 70776851V1 | 339 | 867 |
| 30 | 70779695V1 | 4 | 418 |
| 30 | 5468404H1 | 3 | 275 |
| 30 | 6756043H1 | 102 | 509 |
| 30 | 7190732H1 | 163 | 722 |
| 30 | 71087882V1 | 4 | 560 |
| 30 | 70777729V1 | 4 | 492 |
| 30 | 70781241V1 | 4 | 467 |
| 30 | 70780840V1 | 4 | 442 |
| 30 | 70779497V1 | 342 | 547 |
| 30 | 3845553H1 | 372 | 583 |
| 30 | 5057288F9 | 386 | 1043 |
| 30 | 5057288H1 | 386 | 669 |
| 30 | 70776801V1 | 399 | 933 |
| 30 | 5065461F8 | 408 | 858 |
| 30 | 70778496V1 | 429 | 737 |
| 30 | 70777098V1 | 456 | 752 |
| 30 | 70780976V1 | 476 | 1081 |
| 30 | 70777266V1 | 503 | 1128 |
| 30 | 70776726V1 | 513 | 1093 |
| 30 | 70772943V1 | 541 | 740 |
| 30 | 70777725V1 | 576 | 1166 |
| 30 | 70779028V1 | 613 | 766 |
| 30 | 213833H1 | 1323 | 1388 |
| 30 | 213841T6 | 1323 | 1391 |
| 30 | 213841R6 | 1323 | 1388 |
| 30 | 213841H1 | 1323 | 1388 |
| 30 | 6917202H1 | 10 | 263 |
| 30 | 3385663H1 | 1 | 261 |
| 30 | 5468404F8 | 3 | 596 |
| 30 | 3385663F6 | 1 | 319 |
| 30 | 70775648V1 | 4 | 416 |
| 30 | 70778292V1 | 4 | 577 |
| 30 | 70779496V1 | 4 | 568 |
| 30 | 70775549V1 | 4 | 538 |
| 30 | 70776773V1 | 4 | 576 |
| 31 | g3254719 | 1 | 445 |
| 31 | g5745388 | 1 | 462 |
| 31 | g3959110 | 1 | 353 |
| 31 | g897505 | 1 | 215 |
| 31 | g5233046 | 1 | 295 |
| 31 | g2929868 | 1 | 406 |
| 31 | g3016800 | 1 | 358 |
| 31 | 6202689H1 | 1 | 548 |
| 31 | g2435161 | 9 | 456 |
| 31 | 4707801H1 | 1197 | 1296 |
| 31 | 806768H1 | 1211 | 1421 |
| 31 | 6915008H1 | 1320 | 1761 |
| 31 | 3036834H1 | 1578 | 1846 |
| 31 | 3036834F6 | 1578 | 2016 |
| 31 | 5387714H1 | 1632 | 1886 |
| 31 | 7122625H1 | 443 | 835 |
| 31 | 6915116J1 | 622 | 1129 |
| 31 | 5092947H1 | 899 | 1177 |
| 31 | 5370334H1 | 939 | 1050 |
| 31 | 5092947F6 | 899 | 1330 |
| 31 | 5017752H1 | 959 | 1229 |
| 31 | 4707801F6 | 1197 | 1714 |
| 31 | 5505129H1 | 17 | 282 |
| 31 | 5514010H1 | 17 | 254 |
| 31 | 5505122H1 | 17 | 228 |
| 31 | 1225071H1 | 52 | 262 |
| 31 | g2929769 | 56 | 178 |
| 31 | 6314660H1 | 60 | 613 |
| 31 | 2682720F6 | 173 | 606 |
| 31 | 6516656H1 | 275 | 736 |
| 31 | 6201718H1 | 312 | 599 |
| 31 | 2682720H1 | 324 | 606 |
| 32 | 3665294H1 | 706 | 823 |
| 32 | g1139437 | 903 | 977 |
| 32 | 3663680H1 | 706 | 950 |
| 32 | 5040414H1 | 731 | 947 |
| 32 | g1139431 | 904 | 977 |
| 32 | 1772915R6 | 911 | 1308 |
| 32 | 1772915H1 | 911 | 966 |
| 32 | 4849209H1 | 1000 | 1259 |
| 32 | 1772915T6 | 1024 | 1540 |
| 32 | g4691031 | 1318 | 1588 |
| 32 | 6298418H1 | 1 | 314 |
| 32 | 3403590H1 | 1 | 232 |
| 32 | 6327716H1 | 98 | 415 |
| 32 | g1068995 | 238 | 588 |
| 32 | 6921019H1 | 255 | 457 |
| 32 | 4307678H1 | 254 | 563 |
| 32 | g1858272 | 267 | 664 |
| 32 | 2812311H1 | 503 | 816 |
| 32 | 2812294H1 | 505 | 826 |
| 32 | 4976432H1 | 536 | 792 |
| 32 | g5037963 | 518 | 976 |
| 32 | 4976496H1 | 536 | 801 |
| 32 | g3736768 | 525 | 978 |
| 32 | g4986041 | 540 | 979 |
| 32 | 4124067H1 | 552 | 792 |
| 32 | g4073888 | 527 | 978 |
| 32 | g5526438 | 564 | 978 |
| 32 | 5209523H1 | 413 | 705 |
| 32 | 4109819H1 | 416 | 696 |
| 32 | 3726063H1 | 426 | 688 |
| 32 | 4671035H1 | 421 | 693 |
| 32 | 2311003H1 | 454 | 685 |
| 32 | 5206768H1 | 478 | 723 |
| 32 | 679406H1 | 480 | 737 |
| 32 | 829499H1 | 273 | 519 |
| 32 | 2655709T6 | 279 | 932 |
| 32 | 1494347H1 | 285 | 504 |
| 32 | 042027H1 | 310 | 561 |
| 32 | g1291521 | 316 | 662 |
| 32 | 030668H1 | 319 | 524 |
| 32 | g897225 | 340 | 705 |
| 32 | g921054 | 340 | 660 |
| 32 | g897232 | 340 | 676 |
| 32 | 5018641T6 | 368 | 996 |
| 32 | g2933440 | 374 | 696 |
| 32 | 1876772T6 | 379 | 940 |
| 32 | g2703395 | 564 | 979 |
| 32 | 2652012T6 | 566 | 930 |
| 32 | g1858336 | 575 | 1000 |
| 32 | 3124683H1 | 622 | 881 |
| 32 | g1313829 | 631 | 867 |
| 32 | 4200739H1 | 671 | 956 |
| 32 | 2244549H1 | 671 | 882 |
| 32 | 2684460H1 | 696 | 909 |
| 33 | g3232386 | 1 | 362 |
| 33 | g3181700 | 7 | 419 |
| 33 | 6109493H1 | 322 | 613 |
| 34 | 3081155F6 | 1 | 417 |
| 34 | 3081155H1 | 2 | 315 |
| 34 | 3081155T6 | 31 | 463 |
| 34 | g1648354 | 58 | 455 |
| 34 | 5800009H1 | 103 | 522 |
| 35 | 3517533R6 | 332 | 653 |
| 35 | 3517533H1 | 332 | 621 |
| 35 | g5178468 | 190 | 667 |
| 35 | 3504115H1 | 162 | 477 |
| 35 | 3082646H1 | 123 | 415 |
| 35 | 259700H1 | 1 | 278 |
| 35 | 7169509H1 | 1 | 477 |
| 35 | 6768733H1 | 58 | 292 |
| 35 | 3176620H1 | 66 | 320 |
| 35 | 3176620F6 | 66 | 551 |
| 35 | 4613367H1 | 72 | 326 |
| 36 | 6753724J1 | 1 | 550 |
| 36 | 6769683J1 | 1 | 595 |
| 36 | 6772986J1 | 3 | 579 |
| 36 | 6769153J1 | 1 | 529 |
| 36 | 6764824J1 | 3 | 589 |
| 36 | g6048166 | 3 | 423 |
| 37 | 653157H1 | 82 | 310 |
| 37 | 509350H1 | 107 | 307 |
| 37 | 3386816H1 | 1 | 183 |
| 37 | 3888124H1 | 4 | 268 |
| 37 | 6779840H1 | 9 | 549 |
| 37 | 3159561H1 | 58 | 220 |
| 37 | 6250084H1 | 67 | 593 |
| 37 | 2809974H1 | 67 | 324 |
| 37 | 3386816F6 | 1 | 622 |
| 37 | 3672749H1 | 81 | 364 |
| 37 | 4005442H1 | 2 | 285 |
| 37 | 70821167V1 | 627 | 1066 |
| 37 | 70822737V1 | 643 | 949 |
| 37 | 70832705V1 | 715 | 823 |
| 37 | 412478T6 | 835 | 1013 |
| 37 | 6779840J1 | 894 | 1315 |
| 37 | g2631213 | 986 | 1049 |
| 37 | 5501816H1 | 1053 | 1261 |
| 37 | 5501816F6 | 1053 | 1293 |
| 37 | 4005442T6 | 487 | 1008 |
| 37 | 70818940V1 | 506 | 1068 |
| 37 | 059074H1 | 601 | 765 |
| 37 | 2862291H1 | 375 | 652 |
| 37 | g3934153 | 376 | 841 |
| 37 | g3900180 | 404 | 840 |
| 37 | 391612F1 | 432 | 1051 |
| 37 | g895347 | 1 | 84 |
| 37 | 4005442F6 | 1 | 326 |
| 37 | 391612R1 | 353 | 898 |
| 37 | 412478R6 | 373 | 699 |
| 37 | 412478H1 | 373 | 600 |
| 37 | 3386816T6 | 337 | 818 |
| 37 | 1951349T6 | 296 | 802 |
| 37 | 6947424H1 | 302 | 813 |
| 38 | 6980110H1 | 1 | 501 |
| 38 | g1897936 | 277 | 634 |
| 38 | 6264019H1 | 282 | 417 |
| 38 | 3099905H1 | 300 | 614 |
| 38 | g2329399 | 438 | 689 |
| 38 | 2821248F6 | 452 | 766 |
| 38 | 2821248H1 | 452 | 755 |
| 38 | g954493 | 494 | 556 |
| 38 | 6291249H1 | 507 | 718 |
| 38 | 5636134H1 | 640 | 912 |
| 38 | 6758395H1 | 658 | 1132 |
| 38 | 1903638H1 | 701 | 954 |
| 38 | 4991026H1 | 847 | 1075 |
| 38 | 4991026F6 | 847 | 1335 |
| 38 | 2821248T6 | 894 | 1434 |
| 38 | 6208066H1 | 893 | 1387 |
| 38 | 4453533H2 | 942 | 1149 |
| 38 | 5621950H1 | 1017 | 1297 |
| 38 | 6827666H1 | 1089 | 1614 |
| 38 | 3165907H1 | 1298 | 1569 |
| 38 | 4982360H1 | 1350 | 1616 |
| 39 | 5998240H1 | 1 | 484 |
| 39 | 458061H1 | 20 | 259 |
| 39 | 455162H1 | 20 | 272 |
| 39 | 460544R6 | 20 | 326 |
| 39 | 455966H1 | 20 | 255 |
| 39 | 455162R6 | 20 | 521 |
| 39 | 460544H1 | 20 | 245 |
| 39 | 3206542H1 | 25 | 202 |
| 39 | 4663964H1 | 37 | 289 |
| 39 | g1807165 | 81 | 278 |
| 39 | g2401959 | 129 | 486 |
| 39 | 3614680H1 | 155 | 272 |
| 39 | g5101146 | 180 | 486 |
| 40 | 6927079H1 | 1 | 400 |
| 40 | g922215 | 84 | 386 |
| 40 | g922268 | 84 | 327 |
| 40 | g991321 | 109 | 483 |
| 40 | 4991883T6 | 268 | 799 |
| 40 | 1958936H1 | 672 | 848 |
| 41 | 6033171H1 | 1 | 434 |
| 41 | g1648352 | 23 | 428 |
| 41 | 3023715H1 | 184 | 448 |
| 41 | 2071327F6 | 285 | 584 |
| 41 | 2071327H1 | 285 | 475 |
| 41 | 6912958J1 | 351 | 944 |
| 41 | 4583753H1 | 378 | 666 |
| 41 | 2071327T6 | 676 | 1111 |
| 41 | g4176194 | 713 | 1156 |
| 41 | g3178308 | 731 | 1151 |
| 41 | g4149782 | 824 | 1142 |
| 41 | 6912958H1 | 837 | 1307 |
| 41 | g3797002 | 974 | 1088 |
| 41 | g3180193 | 1135 | 1214 |
| 42 | 6937854H1 | 1927 | 2414 |
| 42 | 7032292H1 | 1837 | 2263 |
| 42 | 7002267H1 | 1385 | 1996 |
| 42 | 7153478H1 | 1986 | 2456 |
| 42 | 7100968H1 | 1404 | 1874 |
| 42 | 7035269H1 | 1372 | 1791 |
| 42 | 7166089H1 | 1147 | 1690 |
| 42 | 5637088H1 | 1339 | 1516 |
| 42 | 6778731H1 | 949 | 1515 |
| 42 | 2689778H1 | 1187 | 1439 |
| 42 | 4293860F6 | 818 | 1413 |
| 42 | 5975002H1 | 2124 | 2431 |
| 42 | g1423371 | 869 | 1290 |
| 42 | g3424813 | 874 | 1290 |
| 42 | g1859062 | 1009 | 1290 |
| 42 | 1809216F6 | 809 | 1271 |
| 42 | 1809216H1 | 1109 | 1271 |
| 42 | 4290553H1 | 911 | 1161 |
| 42 | 6932930H1 | 733 | 1101 |
| 42 | g1858731 | 690 | 1070 |
| 42 | 4293860H1 | 818 | 1065 |
| 42 | 4042230H1 | 673 | 849 |
| 42 | g1423374 | 236 | 722 |
| 42 | 6132451H1 | 275 | 568 |
| 42 | 6781622H1 | 1 | 541 |
| 42 | 5966128H1 | 192 | 357 |
| 43 | 117874R6 | 1 | 438 |
| 43 | 4229003F6 | 171 | 674 |
| 43 | 4229003T6 | 271 | 738 |
| 43 | g3895952 | 404 | 823 |
| 44 | g3803941 | 113 | 583 |
| 44 | g3805589 | 75 | 524 |
| 44 | g3917967 | 73 | 520 |
| 44 | 3817246H1 | 28 | 320 |
| 44 | 2895718H1 | 1 | 283 |
| 45 | 4295967F8 | 1 | 404 |
| 45 | g5811741 | 166 | 582 |
| 46 | 6708077H1 | 547 | 1179 |
| 46 | 5352502H1 | 578 | 664 |
| 46 | 6819988J1 | 1 | 605 |
| 46 | 6292367H1 | 493 | 609 |
| 46 | g4900516 | 542 | 664 |
| 47 | 2607525T6 | 1 | 557 |
| 48 | 5120882F6 | 1 | 353 |
| 48 | g4072524 | 256 | 619 |
| 49 | 4936033R6 | 591 | 1027 |
| 49 | 5387945T6 | 1 | 633 |
| 50 | 5401502T6 | 1 | 364 |
| 51 | 2205615T6 | 377 | 901 |
| 51 | 4150193F6 | 1 | 509 |
| 52 | g2005559 | 1 | 239 |
| 52 | 1399832H1 | 1 | 227 |
| 52 | 1398471H1 | 1 | 238 |
| 52 | g2027783 | 1 | 329 |
| 52 | 1322674H1 | 2 | 254 |
| 52 | g2278640 | 13 | 442 |
| 52 | g2159473 | 86 | 551 |
| 52 | g2162158 | 123 | 594 |
| 52 | g2156015 | 131 | 366 |
| 52 | g2156002 | 208 | 366 |
| 52 | g1954430 | 228 | 425 |
| 53 | 2737435F6 | 1 | 479 |
| 53 | 2737435T6 | 398 | 597 |
| 54 | 4654776T6 | 126 | 637 |
| 54 | 3796761F6 | 1 | 403 |
| 54 | 3796761H1 | 1 | 306 |
| 54 | 4654776H1 | 34 | 285 |
| 54 | 4654776F6 | 34 | 415 |
| 54 | 1729735H1 | 52 | 280 |
| 54 | g895564 | 59 | 388 |
| 55 | 2920075R6 | 1 | 427 |
| 55 | 784499T6 | 20 | 470 |
| 55 | 2920075H1 | 169 | 427 |
| 55 | 269219H1 | 211 | 414 |
| 55 | 269219R1 | 212 | 754 |
| 55 | g1978467 | 356 | 665 |
| 56 | 6183766H1 | 1 | 267 |
| 56 | g1897936 | 1 | 354 |
| 56 | 3099905H1 | 19 | 334 |
| 56 | g2329399 | 158 | 409 |
| 56 | 2821248F6 | 172 | 486 |
| 56 | 2821248H1 | 172 | 475 |
| 56 | g954493 | 214 | 276 |
| 56 | 6291249H1 | 227 | 438 |
| 56 | 5636134H1 | 360 | 632 |
| 56 | 1903638H1 | 421 | 674 |
| 56 | 4991026F6 | 567 | 1055 |
| 56 | 4991026H1 | 567 | 795 |
| 56 | 6208066H1 | 659 | 1163 |
| 56 | 2821248T6 | 614 | 1154 |
| 56 | 4453533H2 | 662 | 869 |
| 56 | 5621950H1 | 737 | 1017 |
| 56 | 3165907H1 | 1018 | 1289 |
| 56 | 4982360H1 | 1070 | 1335 |
| 56 | 6417723H1 | 1325 | 1610 |
| 57 | 6061626H1 | 753 | 1059 |
| 57 | 5425067H1 | 798 | 1041 |
| 57 | 6331039H1 | 50 | 618 |
| 57 | 6132218H1 | 689 | 951 |
| 57 | 3499278F6 | 482 | 891 |
| 57 | 3499278H1 | 645 | 891 |
| 57 | 4657265H1 | 310 | 411 |
| 57 | 6309037H1 | 233 | 724 |
| 57 | 6538713H1 | 284 | 682 |
| 57 | g1962414 | 304 | 564 |
| 57 | g1376321 | 264 | 526 |
| 57 | g4110945 | 3 | 417 |
| 57 | g4310623 | 1 | 416 |
| 57 | g2733078 | 2 | 384 |
| 57 | g2835164 | 3 | 383 |
| 57 | g3241486 | 1 | 382 |
| 57 | g1376322 | 2 | 296 |
| 57 | 3351668F6 | 1 | 267 |
| 57 | 3351668H1 | 1 | 264 |
| 57 | 1785191H1 | 10 | 194 |
| 57 | g2198029 | 1 | 129 |
| 57 | 4050289H1 | 28 | 127 |
| 57 | 4144289H1 | 20 | 125 |
| 58 | g1979821 | 1 | 188 |
| 58 | g3835204 | 1 | 325 |
| 58 | g4072180 | 19 | 468 |
| 58 | 3519808H1 | 81 | 367 |
| 58 | g843784 | 232 | 499 |
| 58 | 1614746F6 | 288 | 732 |
| 58 | 1614746H1 | 288 | 505 |
| 58 | 1575232H1 | 365 | 577 |
| 58 | g2209653 | 403 | 912 |
| 58 | 6322462H1 | 520 | 718 |
| 58 | 2892611T6 | 645 | 1136 |
| 58 | 1614746T6 | 733 | 1148 |
| 58 | g3644249 | 766 | 1189 |
| 58 | 670448R6 | 800 | 1186 |
| 58 | 677184R6 | 800 | 1152 |
| 58 | 670448T6 | 800 | 1149 |
| 58 | 677184H1 | 800 | 1071 |
| 58 | 670448H1 | 800 | 1055 |
| 58 | g2810503 | 840 | 1186 |
| 58 | g2336018 | 907 | 1187 |
| 58 | 2892611H1 | 945 | 1178 |
| 58 | 1289991H1 | 1081 | 1187 |
| 59 | g3838936 | 83 | 488 |
| 59 | g4085429 | 126 | 588 |
| 59 | g1148294 | 133 | 524 |
| 59 | g3057535 | 162 | 618 |
| 59 | 6429507H1 | 305 | 589 |
| 59 | 2865771F6 | 460 | 851 |
| 59 | 2865771H1 | 460 | 746 |
| 59 | 3639153H1 | 501 | 824 |
| 59 | g1258980 | 584 | 839 |
| 59 | 3569914F6 | 601 | 1111 |
| 59 | 3569914H1 | 601 | 894 |
| 59 | 3569914T6 | 962 | 1569 |
| 59 | 5327014H1 | 1048 | 1291 |
| 59 | 1320957H1 | 1120 | 1353 |
| 59 | 2971828F6 | 1216 | 1745 |
| 59 | g3049125 | 1226 | 1757 |
| 59 | 3077923H1 | 1284 | 1572 |
| 59 | g4069747 | 1301 | 1758 |
| 59 | g3417824 | 1307 | 1758 |
| 59 | 5732379H1 | 1398 | 1646 |
| 59 | 5732479H1 | 1399 | 1592 |
| 59 | 2971828H2 | 1452 | 1745 |
| 59 | g1201083 | 1580 | 1748 |
| 59 | 4459657H1 | 1655 | 1775 |
| 60 | 60206922U1 | 296 | 954 |
| 60 | 368741T6 | 399 | 902 |
| 60 | 368741R6 | 1 | 397 |
| 60 | 368741H1 | 1 | 308 |
| 60 | g4187853 | 375 | 750 |
| 60 | 368741R1 | 1 | 490 |
| 60 | g3838805 | 409 | 751 |
| 61 | 5963989H1 | 1 | 502 |
| 61 | 3593809H1 | 1 | 298 |
| 61 | g1059739 | 1 | 137 |
| 61 | 1850713F6 | 1 | 298 |
| 61 | 1850713H1 | 1 | 241 |
| 62 | 2872408F6 | 1 | 397 |
| 62 | 2872408H1 | 367 | 653 |
| 62 | 2872408T6 | 33 | 629 |
| 63 | 2862618T6 | 1 | 569 |
| 63 | 2838402H2 | 1 | 252 |
| 63 | 287364H1 | 1723 | 2056 |
| 63 | 2867867H1 | 198 | 481 |
| 63 | 2828380H1 | 522 | 792 |
| 63 | 2881835T6 | 606 | 1167 |
| 64 | 2882345T6 | 1809 | 2320 |
| 64 | 2820130T6 | 1421 | 1994 |
| 64 | 2837371H1 | 450 | 548 |
| 65 | 2222227T6 | 809 | 1284 |
| 66 | 270908H1 | 1 | 102 |
| 66 | 2698411H1 | 102 | 402 |
| 66 | 269967H1 | 55 | 354 |
| 66 | 2778926H1 | 24 | 260 |
| 66 | 2737435H1 | 1 | 207 |
| 66 | 2737435F6 | 19 | 501 |
| 66 | 2753160H1 | 1 | 119 |
| 66 | 2718845H1 | 1 | 245 |
| 66 | 2718845F6 | 1 | 395 |
| 66 | 2729146H1 | 107 | 344 |
| 66 | 2690266H1 | 126 | 385 |
| 66 | 2782984H1 | 438 | 571 |
| 66 | 2737435T6 | 420 | 617 |
| 67 | 2666231T6 | 1 | 523 |
| 68 | 1729868T6 | 1414 | 1975 |
| 69 | 3256037T6 | 1397 | 1940 |
| 70 | 3394349T6 | 33 | 600 |
| 71 | 5387945T6 | 685 | 1317 |
| 72 | 394892F1 | 793 | 1443 |
| 72 | 402386T6 | 930 | 1401 |
| 73 | 3318809F6 | 1 | 438 |
| 73 | 3318809H1 | 3 | 276 |
| 73 | 3318809T6 | 227 | 754 |
| 73 | 3255863H1 | 438 | 688 |
| 74 | 3685359H1 | 1 | 303 |
| 74 | 3685359F6 | 42 | 501 |
| 74 | 3685359T6 | 1 | 279 |
| 75 | 3637162H1 | 1 | 292 |
| 75 | 3637162F6 | 38 | 490 |
| 75 | 3699637H1 | 22 | 182 |
| 75 | 3637162T6 | 192 | 740 |
| 76 | 2007652H1 | 225 | 411 |
| 76 | 2007652T6 | 225 | 449 |
| 76 | 2007652R6 | 225 | 480 |
| 77 | 3325402T7 | 1764 | 2221 |
| 78 | 4654776F6 | 34 | 415 |
| 78 | 4654776H1 | 34 | 285 |
| 78 | 4654776T6 | 126 | 637 |
| 79 | 1476477F6 | 1 | 548 |
| 79 | 1476477H1 | 1 | 205 |
| 79 | 1476477H6 | 1 | 207 |
| 79 | 1476485H1 | 1 | 85 |
| 79 | 1476477T6 | 120 | 639 |
| 79 | 1500139H1 | 86 | 292 |
| 79 | 1496323H1 | 591 | 830 |
| 80 | 5200862H1 | 1 | 220 |
| 80 | 5200862F6 | 1 | 508 |
| 80 | 5200862T6 | 371 | 989 |
| 81 | 450278R6 | 619 | 1125 |
| 81 | 448904R6 | 1 | 390 |
| 81 | 450278R7 | 619 | 1046 |
| 81 | 448904H1 | 1 | 216 |
| 81 | 446282H1 | 1 | 256 |
| 81 | 450278H1 | 1 | 198 |
| 81 | 448904T6 | 721 | 1298 |
| 81 | 450278T7 | 788 | 1297 |
| 82 | 807409T6 | 210 | 590 |
| 82 | 807409R6 | 210 | 629 |
| 82 | 807409H1 | 1 | 78 |
| 83 | 3844814F6 | 147 | 721 |
| 83 | 3844814H1 | 433 | 721 |
| 83 | 3779417H1 | 1 | 204 |
| 83 | 3769201H1 | 63 | 208 |
| 83 | 3844814T6 | 63 | 317 |
| 84 | 1224910R6 | 1 | 543 |
| 84 | 1224910H1 | 881 | 1024 |
| 84 | 1224910T6 | 98 | 446 |
| 85 | 5296190T6 | 1523 | 1959 |
| 86 | 70375800D1 | 365 | 741 |
| 86 | 5884784H1 | 1617 | 1830 |
| 86 | 60203053V1 | 286 | 730 |
| 86 | 60203050V1 | 304 | 692 |
| 86 | 70374630D1 | 304 | 668 |
| 86 | 60209436U1 | 1 | 656 |
| 86 | 70373679D1 | 378 | 626 |
| 86 | 70373094D1 | 400 | 786 |
| 86 | 70375597D1 | 303 | 605 |
| 86 | 70373898D1 | 304 | 437 |
| 86 | 70373514D1 | 304 | 437 |
| 86 | 819073H1 | 1256 | 1510 |
| 86 | 2831185H1 | 1233 | 1479 |
| 86 | 70375782D1 | 380 | 772 |
| 86 | 70374947D1 | 304 | 772 |
| 86 | g824801 | 1579 | 1852 |
| 86 | 70376281D1 | 472 | 766 |
| 86 | 60209435U1 | 102 | 747 |
| 86 | g2780043 | 1733 | 2066 |
| 86 | 2751856R6 | 1651 | 2039 |
| 86 | 70374895D1 | 404 | 947 |
| 86 | 70376064D1 | 393 | 947 |
| 86 | 70374077D1 | 472 | 947 |
| 86 | 70373091D1 | 669 | 947 |
| 86 | 70373401D1 | 782 | 947 |
| 86 | 70376523D1 | 789 | 947 |
| 86 | 70375900D1 | 868 | 946 |
| 86 | 3999158H1 | 1844 | 1992 |
| 86 | 70376383D1 | 606 | 867 |
| 86 | 2751856H1 | 1651 | 1923 |
| 86 | 70374800D1 | 304 | 837 |
| 86 | 2751062H1 | 1651 | 1919 |
| 86 | 70373252D1 | 300 | 800 |
| 86 | 70375702D1 | 340 | 800 |
| 86 | 60203054V1 | 353 | 791 |
| 86 | 70373230D1 | 359 | 787 |
| 86 | 5822141H1 | 1871 | 2175 |
| 86 | 6332621H1 | 626 | 1098 |
| 86 | 1689922F6 | 1942 | 2175 |
| 86 | 5820119H1 | 1871 | 2166 |
| 86 | g4222747 | 1715 | 2166 |
| 86 | g3594875 | 1710 | 2160 |
| 86 | g3751911 | 1700 | 2160 |
| 86 | g3847457 | 1754 | 2160 |
| 86 | g2675135 | 1863 | 2160 |
| 86 | g4524280 | 1891 | 2160 |
| 86 | g3422353 | 1857 | 2153 |
| 86 | 1689922H1 | 1942 | 2151 |
| 86 | g1501747 | 1770 | 2146 |
| 86 | g2342389 | 1928 | 2145 |
| 86 | g846897 | 1832 | 2132 |
| 86 | g4264802 | 1694 | 2117 |
| 86 | g3693061 | 1788 | 2117 |
| 86 | g4269694 | 1799 | 2117 |
| 86 | g2269848 | 1756 | 2117 |
| 86 | g824800 | 1834 | 2117 |
| 86 | g3069508 | 1651 | 2117 |
| 86 | g2055928 | 1783 | 2117 |
| 86 | g2539104 | 1673 | 2117 |
| 86 | g2901266 | 2005 | 2113 |
| 86 | 70375731D1 | 590 | 1018 |
| 86 | g4390710 | 1751 | 2184 |
| 87 | 5059054H1 | 1 | 257 |
| 87 | 5059022F9 | 1 | 581 |
| 87 | 179544R6 | 84 | 529 |
| 87 | 1227952T6 | 125 | 595 |
| 87 | 064418H1 | 286 | 457 |
| 87 | 179544H1 | 329 | 529 |
| 87 | g1011391 | 482 | 807 |
| 87 | 4667922H1 | 539 | 759 |
| 88 | g4189831 | 12 | 63 |
| 88 | 4721701H1 | 360 | 439 |
| 88 | 3123901H1 | 1 | 66 |
| 88 | 6785315H1 | 12 | 481 |
| 88 | 7201126H2 | 51 | 570 |
| 89 | 2327449H1 | 15 | 248 |
| 89 | 2327457T6 | 1 | 364 |
| 89 | 2327449T6 | 1 | 288 |
| 89 | 2327449R6 | 13 | 408 |
| 89 | 2327457R6 | 13 | 402 |
| 90 | g3694145 | 26 | 408 |
| 90 | 6779728J1 | 1 | 370 |
| 90 | g3250058 | 1 | 408 |
| 90 | 6245903H1 | 120 | 532 |
| 90 | g4333202 | 140 | 407 |
| 91 | 1895443H1 | 19 | 272 |
| 91 | 4729485H1 | 86 | 235 |
| 91 | 5217490H1 | 1 | 198 |
| 91 | 1542833H1 | 1 | 137 |
| 91 | 3836068F6 | 7 | 546 |
| 91 | 1891884H1 | 19 | 264 |
| 91 | 3052342H1 | 46 | 335 |
| 91 | 5735738H1 | 78 | 348 |
| 91 | g5661058 | 155 | 347 |
| 91 | 3525414H1 | 171 | 494 |
| 91 | 3836068H1 | 7 | 289 |
| 92 | 1335071H1 | 515 | 737 |
| 92 | 1353732H1 | 535 | 775 |
| 92 | 6166536H1 | 736 | 1266 |
| 92 | 6483617F9 | 808 | 1320 |
| 92 | 6483617F8 | 828 | 1386 |
| 92 | 4326477F6 | 1 | 381 |
| 92 | 4326477H1 | 2 | 160 |
| 92 | 4140846T9 | 26 | 550 |
| 92 | 3888314H1 | 52 | 314 |
| 92 | 7073510H1 | 119 | 689 |
| 92 | 7081835H1 | 125 | 678 |
| 92 | 2079383H1 | 301 | 563 |
| 92 | 6405368H1 | 315 | 599 |
| 92 | 7254119H1 | 491 | 935 |
| 92 | 6420281H1 | 653 | 1159 |
| 92 | 6759433J1 | 668 | 1118 |
| 92 | 6483617H1 | 808 | 1319 |
| 93 | 257941R6 | 1 | 252 |
| 93 | 2764170H1 | 19 | 259 |
| 93 | 7040554H1 | 149 | 692 |
| 93 | 6477351H1 | 541 | 1039 |
| 93 | g3163349 | 590 | 1022 |
| 93 | 3209591H1 | 1 | 149 |
| 93 | 5511002H1 | 628 | 880 |
| 93 | 5767313H1 | 1 | 246 |
| 93 | 000413H1 | 1 | 241 |
| 93 | 257941H1 | 1 | 237 |
| 93 | 2727658H1 | 1 | 173 |
| 93 | 5511002F6 | 628 | 905 |
| 93 | g1977359 | 11 | 218 |
| 94 | 894136H1 | 1 | 167 |
| 94 | 893591H1 | 1 | 280 |
| 94 | 3685359F6 | 21 | 480 |
| 94 | 3685359H1 | 21 | 323 |
| 94 | 3685359T6 | 243 | 521 |
| 95 | g1925745 | 1 | 143 |
| 95 | g2752333 | 16 | 387 |
| 95 | g2881366 | 16 | 370 |
| 95 | 3257022H1 | 28 | 287 |
| 95 | 2380565F6 | 186 | 681 |
| 95 | 2380565H1 | 186 | 401 |
| 95 | 6354028H1 | 192 | 471 |
| 95 | 2809746H1 | 248 | 487 |
| 95 | 5110009H1 | 249 | 500 |
| 95 | 5110002H1 | 249 | 441 |
| 95 | 6354128H1 | 326 | 480 |
| 95 | 1546477H1 | 378 | 584 |
| 95 | 5055767H1 | 565 | 845 |
| 95 | 3413877H1 | 572 | 817 |
| 95 | 2360256R6 | 719 | 1123 |
| 95 | 2360256H1 | 719 | 976 |
| 95 | g1688388 | 781 | 1110 |
| 95 | 1330559H1 | 784 | 1027 |
| 95 | g844802 | 968 | 1283 |
| 95 | g1640803 | 1007 | 1243 |
| 96 | 4895030H1 | 2484 | 2784 |
| 96 | g1941651 | 2486 | 2951 |
| 96 | 70657184V1 | 2503 | 3092 |
| 96 | g658182 | 2512 | 2776 |
| 96 | g2718978 | 2525 | 2989 |
| 96 | g715365 | 2538 | 2875 |
| 96 | 5298571H1 | 2539 | 2819 |
| 96 | 4749511H1 | 2540 | 2838 |
| 96 | 5298771H1 | 2539 | 2804 |
| 96 | 5298612H1 | 2540 | 2664 |
| 96 | 6041370H1 | 2550 | 3199 |
| 96 | g1471133 | 2556 | 2990 |
| 96 | 6555238H1 | 2565 | 3127 |
| 96 | 6556278H1 | 2565 | 3092 |
| 96 | 2913641H1 | 2604 | 2878 |
| 96 | 5825362H1 | 2609 | 3122 |
| 96 | 6400248H1 | 2624 | 2806 |
| 96 | g1719353 | 2634 | 3091 |
| 96 | g1960091 | 2671 | 3185 |
| 96 | 6829782H1 | 2683 | 3131 |
| 96 | 6829782J1 | 2683 | 3131 |
| 96 | g6139947 | 2710 | 2989 |
| 96 | 70839657V1 | 2744 | 3193 |
| 96 | 70657566V1 | 2815 | 3435 |
| 96 | 2367903F6 | 2842 | 3294 |
| 96 | 2367903H1 | 2842 | 3079 |
| 96 | 2598088T6 | 2849 | 3428 |
| 96 | 2370164T6 | 2863 | 3400 |
| 96 | 7339911H1 | 2891 | 3434 |
| 96 | 494011F1 | 2890 | 3445 |
| 96 | 70658592V1 | 2911 | 3534 |
| 96 | 1398685H1 | 2935 | 3187 |
| 96 | 70656953V1 | 2938 | 3555 |
| 96 | 1380634H1 | 2939 | 3189 |
| 96 | 5138789H1 | 2949 | 3229 |
| 96 | 70655570V1 | 2957 | 3552 |
| 96 | 5067594H1 | 2958 | 3147 |
| 96 | 494011T6 | 2971 | 3405 |
| 96 | g2051891 | 2978 | 3448 |
| 96 | 6850945H1 | 2986 | 3519 |
| 96 | g4738083 | 2987 | 3447 |
| 96 | 6489656H1 | 2997 | 3108 |
| 96 | g3419253 | 3004 | 3445 |
| 96 | g5656805 | 3025 | 3445 |
| 96 | g1444847 | 3030 | 3446 |
| 96 | g3416160 | 3047 | 3449 |
| 96 | g1719354 | 3058 | 3454 |
| 96 | g1780421 | 3068 | 3450 |
| 96 | 6937801H1 | 3118 | 3682 |
| 96 | 6500625H1 | 3113 | 3727 |
| 96 | g654349 | 3148 | 3441 |
| 96 | g5747781 | 3156 | 3445 |
| 96 | g758975 | 3157 | 3485 |
| 96 | g564984 | 3156 | 3445 |
| 96 | g1357788 | 3163 | 3465 |
| 96 | g2931035 | 3178 | 3445 |
| 96 | 155875T6 | 3180 | 3849 |
| 96 | 3874011H1 | 3185 | 3455 |
| 96 | g1190099 | 3218 | 3440 |
| 96 | 3117949H1 | 3251 | 3435 |
| 96 | 2756752H1 | 3308 | 3580 |
| 96 | 70837859V1 | 3306 | 3896 |
| 96 | 495759F1 | 3326 | 3893 |
| 96 | 1332986T6 | 3341 | 3399 |
| 96 | 3297117H1 | 3428 | 3687 |
| 96 | g2959267 | 3429 | 3904 |
| 96 | g3836196 | 3462 | 3898 |
| 96 | 154612T6 | 3493 | 3858 |
| 96 | g2877501 | 3517 | 3898 |
| 96 | g3838447 | 3541 | 3898 |
| 96 | g1187596 | 3595 | 3893 |
| 96 | 7004590H1 | 3601 | 3894 |
| 96 | 6192804H1 | 3620 | 3893 |
| 96 | 6194735H1 | 3620 | 3893 |
| 96 | 6194703H1 | 3619 | 3877 |
| 96 | 495759T6 | 3642 | 3849 |
| 96 | g4737879 | 3648 | 3893 |
| 96 | g758939 | 3648 | 3873 |
| 96 | g3895731 | 3648 | 3898 |
| 96 | g1079906 | 3666 | 3944 |
| 96 | 767355H1 | 3801 | 4056 |
| 96 | g1115031 | 3911 | 4343 |
| 96 | 7337250H1 | 1 | 574 |
| 96 | 2485552H1 | 46 | 257 |
| 96 | 7235980H1 | 61 | 574 |
| 96 | g3869258 | 202 | 4338 |
| 96 | g3869256 | 202 | 4338 |
| 96 | 2693067H1 | 559 | 813 |
| 96 | 6945783H1 | 613 | 1122 |
| 96 | 2535607H1 | 838 | 1099 |
| 96 | 70837439V1 | 956 | 1519 |
| 96 | 3502278H1 | 1050 | 1359 |
| 96 | g1779649 | 1215 | 1673 |
| 96 | 60209482U1 | 1264 | 1763 |
| 96 | 495759R1 | 1304 | 1782 |
| 96 | 495759R6 | 1305 | 1682 |
| 96 | 495759H1 | 1305 | 1559 |
| 96 | 70375665D1 | 1438 | 2033 |
| 96 | 60207458U1 | 1487 | 2000 |
| 96 | 60209492U1 | 1489 | 2005 |
| 96 | 60209480U1 | 1489 | 1845 |
| 96 | 7006928H1 | 1535 | 1890 |
| 96 | 70375676D1 | 1644 | 2122 |
| 96 | 70375660D1 | 1684 | 1921 |
| 96 | 494011R1 | 1689 | 2115 |
| 96 | 494011R6 | 1689 | 2062 |
| 96 | 494011H1 | 1689 | 1921 |
| 96 | g3896433 | 1698 | 2078 |
| 96 | 994895R1 | 1733 | 2255 |
| 96 | 994895H1 | 1733 | 2006 |
| 96 | 680754H1 | 1778 | 2047 |
| 96 | 70374292D1 | 1783 | 2008 |
| 96 | 70375422D1 | 1783 | 2241 |
| 96 | 70375842D1 | 1789 | 2222 |
| 96 | 3488220H1 | 1791 | 2075 |
| 96 | 71220762V1 | 1851 | 2480 |
| 96 | 1332986F6 | 1863 | 2182 |
| 96 | 71221685V1 | 1859 | 2529 |
| 96 | 1332986H1 | 1863 | 2097 |
| 96 | 70838337V1 | 1962 | 2525 |
| 96 | 1964279H1 | 1965 | 2251 |
| 96 | 71221831V1 | 2034 | 2530 |
| 96 | 70375269D1 | 2043 | 2527 |
| 96 | g1357787 | 2095 | 2714 |
| 96 | 5874572H1 | 2106 | 2365 |
| 96 | 71221729V1 | 2117 | 2580 |
| 96 | 70838208V1 | 2146 | 2490 |
| 96 | 2598088F6 | 2168 | 2790 |
| 96 | 2598088H1 | 2168 | 2286 |
| 96 | g573567 | 2168 | 2536 |
| 96 | 6409839H1 | 2218 | 2783 |
| 96 | 5035552H1 | 2282 | 2539 |
| 96 | 70658011V1 | 2283 | 2794 |
| 96 | 70656808V1 | 2299 | 2887 |
| 96 | 1997937R6 | 2308 | 2826 |
| 96 | 1997937H1 | 2308 | 2587 |
| 96 | g3887783 | 2344 | 2793 |
| 96 | 1997937T6 | 2397 | 2962 |
| 96 | g1280977 | 2404 | 2950 |
| 96 | 155875H1 | 2404 | 2627 |
| 96 | 155875R6 | 2404 | 2906 |
| 96 | 70839485V1 | 2403 | 2962 |
| 96 | 6096636H1 | 2466 | 2679 |
| 96 | 7126727H1 | 2484 | 3045 |
| 96 | 5876310H1 | 2484 | 2804 |
| 97 | 4719538H1 | 925 | 1185 |
| 97 | 4911350H1 | 964 | 1239 |
| 97 | 1903472H1 | 984 | 1228 |
| 97 | 3639862H1 | 1027 | 1306 |
| 97 | 4541862F6 | 1034 | 1422 |
| 97 | 6907459H1 | 1061 | 1564 |
| 97 | g5657760 | 1076 | 1506 |
| 97 | 6908516J1 | 1077 | 1642 |
| 97 | 4697720H1 | 1125 | 1372 |
| 97 | 4238385H1 | 1 | 283 |
| 97 | 3403365H1 | 73 | 315 |
| 97 | 3403365F6 | 74 | 334 |
| 97 | 6907459J1 | 151 | 705 |
| 97 | 4551324H1 | 169 | 418 |
| 97 | 3374712H1 | 211 | 466 |
| 97 | 60264809D1 | 228 | 424 |
| 97 | 1954023H1 | 257 | 493 |
| 97 | g792057 | 641 | 899 |
| 97 | 60264821D1 | 680 | 1235 |
| 97 | 60264804D1 | 680 | 1220 |
| 97 | 60264819D1 | 680 | 1192 |
| 97 | g2011093 | 754 | 943 |
| 97 | 069791H1 | 807 | 940 |
| 97 | 4916612H1 | 819 | 1108 |
| 97 | 4916612F7 | 828 | 1360 |
| 97 | 4719538F6 | 925 | 1431 |
| 97 | 6337692H1 | 300 | 461 |
| 97 | 3371311H1 | 544 | 811 |
| 97 | 3091243H1 | 1164 | 1445 |
| 97 | 3915431H1 | 1199 | 1503 |
| 97 | 1954644H1 | 1145 | 1429 |
| 97 | 2596935H1 | 1269 | 1503 |
| 97 | 4719538T6 | 1597 | 1835 |
| 97 | g812717 | 1598 | 1774 |
| 97 | 60100951B1 | 1279 | 1689 |
| 97 | 2410012H1 | 1308 | 1528 |
| 97 | 5781437H1 | 1353 | 1643 |
| 97 | 1499912H1 | 1387 | 1582 |
| 97 | 2777329H1 | 1412 | 1639 |
| 97 | 60100952B1 | 1415 | 1827 |
| 97 | g2577280 | 1497 | 1671 |
| 97 | g3648252 | 1574 | 1704 |
| 97 | g1128730 | 1628 | 1854 |
| 97 | g2837364 | 1650 | 1832 |
| 97 | 60100950D1 | 860 | 1177 |
| 97 | 60100952D1 | 860 | 1116 |
| 97 | 1264982H1 | 895 | 1131 |
| 97 | 6120149H1 | 908 | 1402 |
| 97 | 6119056H1 | 908 | 1530 |
| 97 | 60264817D1 | 625 | 1184 |
| 97 | 60100951D1 | 637 | 929 |
| 98 | 6197337H1 | 1 | 377 |
| 98 | 6198216T8 | 1 | 273 |
| 98 | 6197337F8 | 18 | 368 |
| 98 | 6331250H1 | 213 | 825 |
| 99 | 3016435F6 | 1 | 256 |
| 99 | 3016435H1 | 1 | 138 |
| 99 | 5964071H1 | 28 | 318 |
| 99 | 3016435T6 | 183 | 759 |
| 99 | 2094922H1 | 268 | 318 |
| 99 | 4587201H1 | 585 | 849 |
| 99 | 4762091H1 | 613 | 811 |
| 99 | 1495146R6 | 616 | 1100 |
| 99 | 1495146H1 | 616 | 775 |
| 99 | 5326946H1 | 671 | 928 |
| 99 | 1495146T6 | 681 | 1055 |
| 99 | 3703757H1 | 702 | 1004 |
| 99 | 2948181T6 | 719 | 1062 |
| 99 | 2948181F6 | 726 | 1100 |
| 99 | 2948181H1 | 726 | 983 |
| 99 | g4620028 | 745 | 1100 |
| 99 | g928615 | 748 | 1102 |
| 99 | 677653H1 | 1004 | 1100 |
| 100 | 5901489T6 | 1 | 584 |
| 101 | 2025051T6 | 1192 | 1583 |
| 102 | g2932743 | 1 | 465 |
| 102 | 7165328H1 | 224 | 722 |
| 102 | g5664446 | 237 | 597 |
| 102 | 2863343H1 | 346 | 643 |
| 102 | 4030732H1 | 346 | 455 |
| 102 | 2863343F6 | 346 | 769 |
| 102 | 3323244H1 | 597 | 856 |
| 103 | 4987160F6 | 1 | 601 |
| 103 | 4986076H1 | 251 | 398 |
| 103 | 2753343R6 | 273 | 678 |
| 103 | 2753343H1 | 273 | 523 |
| 103 | 4793432H1 | 351 | 482 |
| 103 | 4793224H1 | 353 | 635 |
| 103 | 2753343T6 | 394 | 811 |
| 103 | 4987160H1 | 1 | 271 |
| 103 | 3343341T6 | 430 | 814 |
| 103 | 4228058H1 | 434 | 708 |
| 103 | 864677H1 | 614 | 856 |
| 103 | 2755913T6 | 618 | 813 |
| 103 | 2755913H1 | 618 | 848 |
| 103 | 2755913R6 | 618 | 847 |
| 103 | 1971855T6 | 627 | 812 |
| 103 | 6161690H1 | 661 | 856 |
| 104 | 1006355H1 | 6597 | 6859 |
| 104 | 3957372H2 | 6610 | 6875 |
| 104 | 186558R6 | 7598 | 8102 |
| 104 | g4838144 | 307 | 6193 |
| 104 | g3847721 | 8369 | 8524 |
| 104 | g3861906 | 8369 | 8524 |
| 104 | 185105H1 | 7598 | 7794 |
| 104 | 186558H1 | 7598 | 7745 |
| 104 | 186558T6 | 7859 | 8487 |
| 104 | g1635936 | 7988 | 8197 |
| 104 | 4619766H1 | 8062 | 8337 |
| 104 | 7356046H1 | 8121 | 8524 |
| 104 | g3797365 | 8183 | 8524 |
| 104 | 6432853H1 | 8263 | 8530 |
| 104 | 6434079H1 | 8263 | 8530 |
| 104 | 756968R6 | 8287 | 8524 |
| 104 | 756968H1 | 8287 | 8518 |
| 104 | g184038 | 1 | 8521 |
| 104 | 7000401H1 | 343 | 428 |
| 104 | 1608059F6 | 445 | 836 |
| 104 | 1608059H1 | 445 | 664 |
| 104 | 2169635F6 | 1141 | 1544 |
| 104 | 2169635H1 | 1141 | 1373 |
| 104 | 2169635T6 | 1716 | 2134 |
| 104 | 7229203H1 | 2112 | 2612 |
| 104 | g314259 | 4291 | 4813 |
| 104 | 3960183H1 | 6436 | 6705 |
| 105 | 70524885V1 | 1630 | 2395 |
| 105 | 70524843V1 | 1639 | 2253 |
| 105 | 6643169V1 | 1689 | 1909 |
| 105 | 70646269V1 | 1689 | 1909 |
| 105 | 70525307V1 | 1716 | 2188 |
| 105 | g880693 | 1746 | 2259 |
| 105 | 70533384V1 | 2383 | 2603 |
| 105 | 2707682T6 | 2410 | 2903 |
| 105 | 70523481V1 | 2420 | 2965 |
| 105 | 70525826V1 | 2426 | 2939 |
| 105 | 70525835V1 | 2426 | 2939 |
| 105 | 70523321V1 | 2540 | 2944 |
| 105 | g875595 | 2611 | 2954 |
| 105 | 70523241V1 | 2671 | 2938 |
| 105 | g561027 | 2772 | 2938 |
| 105 | g875594 | 1746 | 2106 |
| 105 | g669502 | 1745 | 1996 |
| 105 | 70525213V1 | 1780 | 2434 |
| 105 | 70522841V1 | 1864 | 2410 |
| 105 | 70524066V1 | 1885 | 2366 |
| 105 | 70524543V1 | 1916 | 2434 |
| 105 | 7238311H1 | 1931 | 2473 |
| 105 | 70522425V1 | 1952 | 2725 |
| 105 | 70525244V1 | 2005 | 2625 |
| 105 | 70526939V1 | 2013 | 2610 |
| 105 | 70526388V1 | 2192 | 2409 |
| 105 | 70524889V1 | 2198 | 2767 |
| 105 | 70526612V1 | 2200 | 2358 |
| 105 | 70522907V1 | 2239 | 2973 |
| 105 | 70522785V1 | 2248 | 2927 |
| 105 | 70526373V1 | 2260 | 2416 |
| 105 | 7086069H1 | 2358 | 2898 |
| 105 | 7071063H1 | 1 | 547 |
| 105 | g913241 | 159 | 2181 |
| 105 | g6299529 | 164 | 599 |
| 105 | 7091369H1 | 245 | 847 |
| 105 | 7347105H1 | 266 | 460 |
| 105 | 5312260H1 | 357 | 578 |
| 105 | 7090770H1 | 1143 | 1683 |
| 105 | 70525682V1 | 1204 | 1896 |
| 105 | 70526748V1 | 1205 | 1699 |
| 105 | 2707682H1 | 1213 | 1477 |
| 105 | 2707682F6 | 1213 | 1439 |
| 105 | 70522990V1 | 1214 | 1837 |
| 105 | 1569986H1 | 1252 | 1374 |
| 105 | 70529862V1 | 1437 | 1903 |
| 105 | 70522200V1 | 1481 | 2029 |
| 105 | 70525480V1 | 1546 | 2185 |
| 105 | 6449560H1 | 1564 | 2157 |
| 106 | 3408296H1 | 1562 | 1812 |
| 106 | 3173959T6 | 1584 | 2175 |
| 106 | 2298374R6 | 1623 | 2077 |
| 106 | 2298374H1 | 1623 | 1895 |
| 106 | 1361272F1 | 1657 | 2216 |
| 106 | 1361366H1 | 1657 | 1834 |
| 106 | 1683557F6 | 1671 | 2227 |
| 106 | 1683573F6 | 1671 | 2086 |
| 106 | 1683573H1 | 1671 | 1907 |
| 106 | 1336611H1 | 1698 | 1941 |
| 106 | 71265365V1 | 1447 | 1892 |
| 106 | 70062844V1 | 1469 | 1865 |
| 106 | 2273538H1 | 1494 | 1763 |
| 106 | 3022474H1 | 1540 | 1833 |
| 106 | 71120676V1 | 1546 | 1932 |
| 106 | 70524882V1 | 1559 | 1725 |
| 106 | 70528634V1 | 558 | 1205 |
| 106 | 2408847H1 | 1882 | 2132 |
| 106 | 70059242V1 | 1888 | 2222 |
| 106 | g5855887 | 1901 | 2223 |
| 106 | g5858285 | 1901 | 2224 |
| 106 | 3409241T6 | 1911 | 2367 |
| 106 | 5669576H1 | 1931 | 2121 |
| 106 | 70062956V1 | 1793 | 2090 |
| 106 | 70061871V1 | 1800 | 2151 |
| 106 | 4778510H1 | 1802 | 2080 |
| 106 | 70061698V1 | 1811 | 2222 |
| 106 | g4901915 | 1816 | 2222 |
| 106 | 70060889V1 | 1815 | 2268 |
| 106 | 1623591T6 | 1819 | 2177 |
| 106 | 1915736T6 | 1818 | 2176 |
| 106 | g3155476 | 1821 | 2208 |
| 106 | g5392637 | 1826 | 2222 |
| 106 | 70061837V1 | 1835 | 2222 |
| 106 | 1623591F6 | 1853 | 2222 |
| 106 | 1623591H1 | 1853 | 2074 |
| 106 | g2198272 | 1858 | 2220 |
| 106 | 960516R1 | 1859 | 2222 |
| 106 | 70062540V1 | 1860 | 2222 |
| 106 | 960516H1 | 1859 | 2160 |
| 106 | 960387T1 | 1859 | 2185 |
| 106 | 2086141H1 | 1864 | 2127 |
| 106 | 4239442T8 | 1884 | 2294 |
| 106 | 5376913H1 | 683 | 950 |
| 106 | 2557738H1 | 650 | 892 |
| 106 | 2560627H1 | 650 | 911 |
| 106 | 1853191F6 | 1706 | 2218 |
| 106 | 1853191H1 | 1706 | 1960 |
| 106 | 1632340H1 | 1705 | 1922 |
| 106 | 614199H1 | 1706 | 1933 |
| 106 | 70059326V1 | 1719 | 2222 |
| 106 | 1683573T6 | 1725 | 2175 |
| 106 | 3751266T6 | 1725 | 2187 |
| 106 | 3321818T6 | 1730 | 2185 |
| 106 | 70062398V1 | 1341 | 1862 |
| 106 | 71118139V1 | 1346 | 1909 |
| 106 | 1865168H1 | 1348 | 1605 |
| 106 | 71294927V1 | 1403 | 1816 |
| 106 | 71120506V1 | 1404 | 1857 |
| 106 | 71118652V1 | 1408 | 2023 |
| 106 | 71266623V1 | 1448 | 1751 |
| 106 | 71265047V1 | 731 | 1261 |
| 106 | 71119759V1 | 731 | 1234 |
| 106 | 71266124V1 | 732 | 1366 |
| 106 | 71118641V1 | 731 | 1311 |
| 106 | 71118731V1 | 731 | 1292 |
| 106 | 71265792V1 | 731 | 1260 |
| 106 | 1915736R6 | 731 | 1137 |
| 106 | 2681128H1 | 696 | 984 |
| 106 | 70531848V1 | 706 | 1260 |
| 106 | 71264971V1 | 725 | 1481 |
| 106 | 71266413V1 | 922 | 1608 |
| 106 | 71117117V1 | 921 | 1580 |
| 106 | 3672409H1 | 937 | 1168 |
| 106 | 71118560V1 | 968 | 1537 |
| 106 | 71119741V1 | 973 | 1452 |
| 106 | 3964084H1 | 1006 | 1294 |
| 106 | 3628624H1 | 1936 | 2251 |
| 106 | g2969311 | 1938 | 2222 |
| 106 | g3151378 | 1960 | 2226 |
| 106 | g1980460 | 1995 | 2313 |
| 106 | 223027R1 | 2064 | 2222 |
| 106 | 223027H1 | 2063 | 2222 |
| 106 | 223027F1 | 2064 | 2222 |
| 106 | 70529734V1 | 461 | 579 |
| 106 | g850466 | 493 | 824 |
| 106 | 70530256V1 | 467 | 1041 |
| 106 | 3751266F6 | 1 | 358 |
| 106 | 3751266H1 | 1 | 296 |
| 106 | 3321818F6 | 103 | 497 |
| 106 | 3321818H1 | 104 | 350 |
| 106 | 6351045H2 | 329 | 660 |
| 106 | 71265775V1 | 801 | 1368 |
| 106 | 4177726H1 | 808 | 1103 |
| 106 | 5296714H1 | 766 | 1037 |
| 106 | 7059995H1 | 799 | 1271 |
| 106 | 096309H1 | 1101 | 1323 |
| 106 | 71118507V1 | 1118 | 1700 |
| 106 | 71120385V1 | 1121 | 1448 |
| 106 | g2198304 | 1133 | 1220 |
| 106 | 71119740V1 | 1134 | 1424 |
| 106 | 4174878H1 | 1137 | 1420 |
| 106 | 71117317V1 | 1153 | 1724 |
| 106 | 71119085V1 | 1197 | 1825 |
| 106 | 3792653H1 | 1224 | 1433 |
| 106 | 6329373H1 | 1235 | 1780 |
| 106 | 71119545V1 | 1267 | 1954 |
| 106 | 71120838V1 | 1275 | 1741 |
| 106 | 4717156H1 | 1277 | 1501 |
| 106 | 5391806H1 | 1282 | 1481 |
| 106 | 71118747V1 | 1295 | 1960 |
| 106 | 70527304V1 | 1301 | 1969 |
| 106 | 71120713V1 | 1306 | 1885 |
| 106 | 71118117V1 | 1311 | 1871 |
| 106 | 5290693H1 | 1314 | 1610 |
| 106 | 5287845H1 | 1314 | 1441 |
| 106 | 70530468V1 | 1320 | 1851 |
| 106 | 3173959F6 | 1322 | 1886 |
| 106 | 70061612V1 | 1322 | 1882 |
| 106 | 70059581V1 | 1322 | 1815 |
| 106 | 70058920V1 | 1322 | 1762 |
| 106 | 70060883V1 | 1322 | 1729 |
| 106 | 2298374T6 | 1735 | 2182 |
| 106 | 70061519V1 | 1747 | 2333 |
| 106 | 3021149H1 | 1758 | 2047 |
| 106 | g2879046 | 1775 | 2225 |
| 106 | 1853191T6 | 1781 | 2376 |
| 106 | g2444573 | 1794 | 2221 |
| 106 | 3934758F6 | 895 | 1411 |
| 106 | 70527491V1 | 846 | 1499 |
| 106 | 71119165V1 | 884 | 1492 |
| 106 | 71265279V1 | 889 | 1575 |
| 106 | 71264808V1 | 753 | 1263 |
| 106 | 71265724V1 | 731 | 1013 |
| 106 | 1915736H1 | 731 | 969 |
| 106 | 71265549V1 | 731 | 1202 |
| 106 | 71266530V1 | 732 | 1246 |
| 106 | 2681128F6 | 695 | 1180 |
| 106 | g2541182 | 2106 | 2424 |
| 106 | g3039868 | 2147 | 2225 |
| 106 | 3931985H1 | 896 | 1182 |
| 106 | 3934538H1 | 895 | 1200 |
| 106 | 3934758H1 | 895 | 1195 |
| 106 | 3934357H1 | 895 | 1200 |
| 106 | 3662984H1 | 1006 | 1254 |
| 106 | 70529227V1 | 1009 | 1601 |
| 106 | 71265619V1 | 1031 | 1689 |
| 106 | 5946829H1 | 1044 | 1325 |
| 106 | 71266345V1 | 1097 | 1749 |
| 106 | 71120487V1 | 1102 | 1594 |
| 106 | 71266010V1 | 1097 | 1765 |
| 106 | 5947885H1 | 1100 | 1363 |
| 106 | 3173959H1 | 1322 | 1589 |
| 106 | 70059533V1 | 1322 | 1585 |
| 106 | g1981676 | 1322 | 1654 |
| 106 | 70062034V1 | 1326 | 1794 |
| 107 | 5913661H1 | 1 | 283 |
| 107 | 5913661F8 | 1 | 569 |
| 107 | 5913661F6 | 1 | 575 |
| 107 | 5913661T6 | 213 | 821 |
| 108 | 6796436H1 | 1 | 435 |
| 108 | g5837313 | 111 | 559 |
| 109 | 2536867F6 | 918 | 1430 |
| 109 | 2536867H2 | 918 | 1187 |
| 109 | g1926046 | 932 | 1379 |
| 109 | g1925836 | 933 | 1201 |
| 109 | 3244923F6 | 993 | 1220 |
| 109 | 3244923H1 | 993 | 1213 |
| 109 | 2878889H1 | 1010 | 1304 |
| 109 | 2878889F6 | 1010 | 1349 |
| 109 | g858504 | 1080 | 1411 |
| 109 | 5567006H1 | 1081 | 1257 |
| 109 | 1896278H1 | 1106 | 1341 |
| 109 | g784668 | 1209 | 1286 |
| 109 | 3364252F6 | 1 | 432 |
| 109 | 3364252H1 | 1 | 226 |
| 109 | 096666H1 | 66 | 233 |
| 109 | 096675H1 | 67 | 240 |
| 109 | 7132087H1 | 255 | 624 |
| 109 | 4030547F8 | 461 | 1001 |
| 109 | 4030547H1 | 462 | 715 |
| 109 | 5077091H1 | 800 | 1072 |
| 110 | 2717953H1 | 1 | 259 |
| 110 | 2806157F6 | 27 | 606 |
| 110 | 2806157H1 | 26 | 323 |
| 110 | 2724233T6 | 367 | 954 |
| 111 | 166942F1 | 1 | 624 |
| 111 | g3755789 | 134 | 505 |
| 111 | g3109437 | 134 | 208 |
| 111 | g3037830 | 135 | 509 |
| 111 | g3180013 | 139 | 583 |
| 111 | g4270829 | 142 | 429 |
| 111 | g3594985 | 142 | 562 |
| 111 | 5282615T6 | 168 | 751 |
| 111 | g2942533 | 248 | 563 |
| 111 | g2953832 | 248 | 509 |
| 111 | g3040122 | 248 | 503 |
| 111 | g3051904 | 251 | 501 |
| 111 | g3804542 | 368 | 750 |
| 111 | 5282615F6 | 635 | 1106 |
| 111 | 5282615H1 | 884 | 1106 |
| 112 | g2563121 | 1 | 166 |
| 112 | 2792728F6 | 1 | 440 |
| 112 | 2792728T6 | 1 | 417 |
| 112 | g3040744 | 1 | 338 |
| 112 | g2714186 | 1 | 401 |
| 112 | 7157205H1 | 1 | 461 |
| 112 | g4371924 | 71 | 205 |
| 112 | 1394888T6 | 90 | 304 |
| 112 | 1624877H1 | 94 | 288 |
| 112 | 2792728H1 | 147 | 439 |
| 113 | g2943715 | 1 | 1450 |
| 113 | 6487571H1 | 657 | 1161 |
| 113 | 6487571F9 | 657 | 1207 |
| 113 | 70681361V1 | 692 | 1244 |
| 113 | 70681601V1 | 692 | 1178 |
| 113 | 1544823R6 | 692 | 1181 |
| 113 | 70681277V1 | 692 | 1164 |
| 113 | 1544823H1 | 692 | 898 |
| 113 | g4686743 | 879 | 1327 |
| 113 | 70680264V1 | 950 | 1079 |
| 113 | 6476403H1 | 998 | 1525 |
| 114 | 6272292H2 | 1 | 507 |
| 114 | 5910821T8 | 365 | 636 |
| 114 | 5910821T9 | 365 | 662 |
| 114 | 5910821F8 | 365 | 793 |
| 114 | 5910821H1 | 365 | 676 |
| 115 | 1551035H1 | 1625 | 1845 |
| 115 | 2716914H1 | 1625 | 1873 |
| 115 | 4876737H1 | 1631 | 1915 |
| 115 | 7091270H1 | 1631 | 1955 |
| 115 | 674945H1 | 1636 | 1903 |
| 115 | 670546H1 | 1636 | 1756 |
| 115 | 2119143H1 | 1637 | 1891 |
| 115 | 2815231H1 | 1643 | 1910 |
| 115 | g990246 | 1645 | 1912 |
| 115 | 957795H1 | 1653 | 1903 |
| 115 | 472488H1 | 1653 | 1882 |
| 115 | 472488R1 | 1654 | 2125 |
| 115 | 302216H1 | 1655 | 1877 |
| 115 | 1876833H1 | 1654 | 1913 |
| 115 | 2697195H1 | 1656 | 1898 |
| 115 | 5377444H1 | 1666 | 1918 |
| 115 | 2431879H1 | 1668 | 1914 |
| 115 | 777024H1 | 1669 | 1901 |
| 115 | 3236114H1 | 1675 | 1928 |
| 115 | 1641892H1 | 1677 | 1880 |
| 115 | 5569350H1 | 1678 | 1920 |
| 115 | 3857806H1 | 1686 | 1976 |
| 115 | g2751499 | 1694 | 2029 |
| 115 | g876527 | 1697 | 2020 |
| 115 | 4069649H1 | 1708 | 1834 |
| 115 | 4307481H1 | 1724 | 1900 |
| 115 | 031299H1 | 1750 | 1920 |
| 115 | 3623362H1 | 1779 | 2032 |
| 115 | g5036497 | 1858 | 2136 |
| 115 | 3624213H1 | 1872 | 2081 |
| 115 | g2411009 | 1907 | 2079 |
| 115 | 4721623H1 | 1918 | 2011 |
| 115 | 2839772H2 | 1922 | 2209 |
| 115 | 6056374H1 | 1933 | 2132 |
| 115 | 6056674H1 | 1933 | 2136 |
| 115 | 3802178H1 | 1933 | 2135 |
| 115 | 4371036H1 | 1940 | 2136 |
| 115 | 412662H1 | 1939 | 2136 |
| 115 | 1907478H1 | 1950 | 2136 |
| 115 | 5050475H1 | 1966 | 2136 |
| 115 | 6447704H1 | 1966 | 2106 |
| 115 | g3888474 | 1988 | 2321 |
| 115 | 5379172H1 | 1989 | 2238 |
| 115 | 5015647H1 | 1994 | 2136 |
| 115 | 3327673H1 | 1995 | 2251 |
| 115 | 3567634H1 | 2021 | 2137 |
| 115 | 1739210H1 | 2025 | 2215 |
| 115 | 880124H1 | 2032 | 2132 |
| 115 | 2354054H1 | 2037 | 2132 |
| 115 | 1235532H1 | 2060 | 2132 |
| 115 | 2350867H1 | 2062 | 2132 |
| 115 | g983285 | 2072 | 2421 |
| 115 | 4190021H1 | 2084 | 2132 |
| 115 | g2017399 | 2116 | 2329 |
| 115 | 6550613H1 | 2153 | 2494 |
| 115 | 5195955H1 | 2155 | 2420 |
| 115 | 3907635H1 | 2185 | 2464 |
| 115 | g3166884 | 2193 | 2356 |
| 115 | g994523 | 2232 | 2476 |
| 115 | g1099949 | 2244 | 2491 |
| 115 | g757333 | 2245 | 2513 |
| 115 | 4534796H1 | 2255 | 2463 |
| 115 | 1543657H1 | 2262 | 2468 |
| 115 | 4754806H1 | 2295 | 2496 |
| 115 | 1947635H1 | 2336 | 2483 |
| 115 | 3930538H1 | 2360 | 2496 |
| 115 | 2760689H1 | 2377 | 2496 |
| 115 | 5563236H1 | 2404 | 2496 |
| 115 | 4310883H1 | 2408 | 2496 |
| 115 | 2778679H1 | 2439 | 2496 |
| 115 | 725638H1 | 1 | 243 |
| 115 | 6733284H1 | 96 | 640 |
| 115 | 3096672H1 | 98 | 410 |
| 115 | 7034622H1 | 100 | 606 |
| 115 | g777461 | 120 | 197 |
| 115 | 5198834H1 | 146 | 385 |
| 115 | 2394482H2 | 157 | 372 |
| 115 | 4970513H1 | 161 | 431 |
| 115 | 4969579H1 | 160 | 361 |
| 115 | 377654H1 | 163 | 374 |
| 115 | 305879H1 | 187 | 444 |
| 115 | 307158H1 | 189 | 433 |
| 115 | 3319063H1 | 215 | 496 |
| 115 | 5504343H1 | 219 | 449 |
| 115 | 3513751H1 | 230 | 473 |
| 115 | 4775727H1 | 230 | 504 |
| 115 | 4696530H1 | 245 | 433 |
| 115 | 928643H1 | 289 | 554 |
| 115 | 4529248H1 | 370 | 618 |
| 115 | 3942806H1 | 418 | 694 |
| 115 | 7280921H1 | 466 | 677 |
| 115 | g2013891 | 480 | 698 |
| 115 | g2013455 | 480 | 709 |
| 115 | 5422141H1 | 481 | 726 |
| 115 | 3110608H1 | 525 | 776 |
| 115 | 5659913H1 | 620 | 896 |
| 115 | 5545664F8 | 647 | 982 |
| 115 | 5545664F6 | 647 | 1087 |
| 115 | 3163564H1 | 751 | 1011 |
| 115 | 761584H1 | 768 | 982 |
| 115 | 6887167J1 | 837 | 1454 |
| 115 | 1852303H1 | 1011 | 1084 |
| 115 | 6948478H1 | 1087 | 1504 |
| 115 | 4158605H1 | 1206 | 1469 |
| 115 | 5270074H1 | 1238 | 1440 |
| 115 | 2365846H1 | 1239 | 1469 |
| 115 | 2444605H1 | 1244 | 1464 |
| 115 | 3115706H1 | 1255 | 1482 |
| 115 | 6728257H1 | 1349 | 1938 |
| 115 | 5717548H1 | 1401 | 1922 |
| 115 | 5796003H1 | 1427 | 1849 |
| 115 | 779492H1 | 1436 | 1699 |
| 115 | 2279382H1 | 1436 | 1702 |
| 115 | 5370473H1 | 1439 | 1656 |
| 115 | 4152676H1 | 1450 | 1712 |
| 115 | 60123909B1 | 1458 | 2086 |
| 115 | 6960268H1 | 1466 | 1902 |
| 115 | 3449619H1 | 1461 | 1706 |
| 115 | 7039654H1 | 1461 | 1970 |
| 115 | 3890456H1 | 1461 | 1758 |
| 115 | 6966324H1 | 1466 | 2051 |
| 115 | 765511H1 | 1469 | 1812 |
| 115 | 4940482T9 | 1481 | 2031 |
| 115 | 6409578H1 | 1486 | 1978 |
| 115 | 6734637H1 | 1495 | 1912 |
| 115 | 2886241H1 | 1493 | 1744 |
| 115 | 1222871H1 | 1495 | 1739 |
| 115 | 4193226H1 | 1498 | 1707 |
| 115 | 7065251H1 | 1500 | 2097 |
| 115 | 2152140H1 | 1501 | 1772 |
| 115 | 3763167H1 | 1502 | 1559 |
| 115 | 3571390H1 | 1504 | 1790 |
| 115 | 942026H1 | 1509 | 1756 |
| 115 | 4533306T1 | 1513 | 2067 |
| 115 | 2124615H1 | 1517 | 1816 |
| 115 | 3684042H1 | 1522 | 1808 |
| 115 | 6715567H1 | 1523 | 2096 |
| 115 | 3322645H1 | 1523 | 1782 |
| 115 | g1873672 | 1524 | 2006 |
| 115 | 3590863H1 | 1530 | 1809 |
| 115 | 60123902B1 | 1538 | 2104 |
| 115 | 1002539H1 | 1538 | 1639 |
| 115 | 6077425H1 | 1541 | 1858 |
| 115 | 6513731H1 | 1548 | 2079 |
| 115 | 3785763H1 | 1548 | 1837 |
| 115 | 538349H1 | 1550 | 1775 |
| 115 | 1832140H1 | 1551 | 1753 |
| 115 | 1669989H1 | 1553 | 1767 |
| 115 | 450252H1 | 1553 | 1773 |
| 115 | 737082H1 | 1556 | 1778 |
| 115 | 7065802H1 | 1560 | 2096 |
| 115 | 6741001H1 | 1563 | 2068 |
| 115 | 835332H1 | 1566 | 1869 |
| 115 | 1599938T6 | 1569 | 2099 |
| 115 | 4226362H1 | 1573 | 1848 |
| 115 | 529930H1 | 1578 | 1721 |
| 115 | 4771124H1 | 1577 | 1846 |
| 115 | 4715169H1 | 1583 | 1859 |
| 115 | 4533768T1 | 1584 | 2101 |
| 115 | 4895425H1 | 1591 | 1861 |
| 115 | 4348930H1 | 1593 | 1852 |
| 115 | 4348733H1 | 1594 | 1854 |
| 115 | g2616416 | 1596 | 1833 |
| 115 | 5698825H1 | 1596 | 1847 |
| 115 | 2288814H1 | 1597 | 1844 |
| 115 | 5762293H1 | 1598 | 2136 |
| 115 | 3082229T6 | 1601 | 1987 |
| 115 | 3737781H1 | 1598 | 1897 |
| 115 | 883098H1 | 1600 | 1835 |
| 115 | 5336847H1 | 1601 | 1837 |
| 115 | 2741220H1 | 1601 | 1860 |
| 115 | 880546H1 | 1600 | 1841 |
| 115 | 6398288H1 | 1601 | 1744 |
| 115 | 3252954H1 | 1608 | 1863 |
| 115 | 3779658H1 | 1618 | 1921 |
| 116 | g1891130 | 669 | 957 |
| 116 | 7333578H1 | 1 | 523 |
| 116 | 6545439H1 | 141 | 676 |
| 116 | g3805536 | 534 | 966 |
| 116 | g3322110 | 534 | 772 |
| 116 | 5610773H1 | 537 | 788 |
| 117 | 6929774H1 | 1 | 513 |
| 117 | 6052078J1 | 72 | 520 |
| 117 | 6052078H1 | 72 | 520 |
| 117 | 4970577H1 | 120 | 381 |
| 117 | 4970577F6 | 120 | 483 |
| 117 | 6292129H1 | 423 | 637 |
| 117 | 6294687H1 | 423 | 647 |
| 117 | 2807905H1 | 555 | 863 |
| 117 | g2540618 | 597 | 871 |
| 117 | 4401727H1 | 650 | 916 |
| 117 | 5729803H1 | 731 | 1236 |
| 117 | g1301257 | 787 | 1245 |
| 117 | 026879H1 | 838 | 1092 |
| 117 | g1303063 | 897 | 1111 |
| 117 | 522135H1 | 1025 | 1160 |
| 117 | 522228H1 | 1025 | 1269 |
| 118 | 587588R6 | 1 | 336 |
| 118 | 587588T6 | 1 | 512 |
| 118 | g1069975 | 229 | 539 |
| 119 | g2809760 | 1 | 443 |
| 119 | g2934256 | 68 | 387 |
| 119 | 2785236H2 | 220 | 481 |
| 119 | 3437984H1 | 274 | 530 |
| 119 | 2544176H1 | 332 | 520 |
| 120 | 2807456F6 | 1 | 508 |
| 120 | 2807456H1 | 1 | 249 |
| 120 | 2807456T6 | 122 | 671 |
| 121 | g3595066 | 1 | 357 |
| 121 | 4665764H1 | 1 | 257 |
| 122 | 70151773V1 | 587 | 917 |
| 122 | 60203477U1 | 266 | 822 |
| 122 | 522228H1 | 955 | 1199 |
| 122 | 522135H1 | 955 | 1090 |
| 122 | 026879H1 | 768 | 1022 |
| 122 | 60203621U1 | 1 | 548 |
| 122 | 60203622U1 | 118 | 507 |
| 122 | 4401727H1 | 579 | 846 |
| 122 | g2540618 | 526 | 801 |
| 122 | 2807905H1 | 484 | 793 |
| 122 | 70152547V1 | 797 | 1219 |
| 122 | 3812508H1 | 140 | 438 |
| 122 | g1265991 | 198 | 338 |
| 122 | 3860472H1 | 256 | 546 |
| 122 | 3520754H1 | 334 | 621 |
| 122 | 70152228V1 | 391 | 1018 |
| 122 | 4350225H1 | 364 | 638 |
| 122 | 70155823V1 | 419 | 986 |
| 122 | g3919706 | 1 | 424 |
| 122 | 2512390F6 | 1 | 316 |
| 122 | g1792877 | 1 | 365 |
| 122 | g4187765 | 4 | 446 |
| 122 | g2905531 | 4 | 102 |
| 122 | 70156040V1 | 960 | 1356 |
| 122 | 70151954V1 | 828 | 1353 |
| 122 | 2512390H1 | 14 | 316 |
| 122 | 60202389B1 | 858 | 1335 |
| 122 | 60202388B1 | 870 | 1329 |
| 122 | 60202388B2 | 924 | 1329 |
| 122 | 999391H1 | 37 | 270 |
| 122 | 2512390T6 | 38 | 315 |
| 122 | 4970577T6 | 45 | 618 |
| 122 | 60110854B2 | 1164 | 1287 |
| 122 | g2884782 | 1 | 452 |
| 122 | g2220423 | 1 | 398 |
| 122 | g1267721 | 1 | 282 |
| 122 | g3918260 | 1 | 411 |
| 123 | 2807456F6 | 194 | 701 |
| 123 | 2807456H1 | 1 | 249 |
| 123 | 2807456T6 | 31 | 580 |
| 123 | 270567H1 | 78 | 161 |
| 123 | 269931H1 | 374 | 499 |
| 123 | 269626H1 | 1824 | 2053 |
| 123 | 269080H1 | 128 | 352 |
| 123 | 270403H1 | 85 | 349 |
| 123 | 270910R1 | 1824 | 2022 |
| 124 | 587588R6 | 220 | 555 |
| 124 | 587588T6 | 44 | 555 |
| 124 | 587588H1 | 1 | 165 |
| 125 | 3321035F6 | 317 | 786 |
| 125 | 3321035H1 | 330 | 595 |
| 125 | g1319620 | 414 | 927 |
| 125 | g2741801 | 414 | 556 |
| 125 | g2841030 | 1354 | 1422 |
| 125 | g2933104 | 421 | 906 |
| 126 | 5259815H1 | 1 | 206 |
| 126 | 3568526H1 | 71 | 366 |
| 126 | 1289824F6 | 181 | 734 |
| 126 | g928730 | 693 | 889 |
| 126 | 764159H1 | 693 | 849 |
| 126 | 6620992H1 | 718 | 1297 |
| 126 | 839936R1 | 825 | 1369 |
| 126 | 1289824H1 | 181 | 349 |
| 126 | 839936H1 | 825 | 1066 |
| 126 | 3869224H1 | 996 | 1286 |
| 126 | 1685280F6 | 522 | 955 |
| 126 | 3223525H1 | 1137 | 1457 |
| 126 | 3843717H1 | 1 | 293 |
| 126 | g1395923 | 1198 | 1531 |
| 126 | 1685280H1 | 522 | 754 |
| 126 | 5028090H1 | 1333 | 1598 |
| 126 | 4216695H1 | 1404 | 1656 |
| 126 | 1289824T6 | 249 | 852 |
| 126 | g2540596 | 600 | 897 |
| 126 | 5724304H1 | 664 | 1233 |
| 126 | 1947742T6 | 688 | 858 |
| 126 | g1225270 | 688 | 889 |
| 126 | 3438058F6 | 269 | 562 |
| 126 | 3438058H1 | 318 | 562 |
| 126 | 3438058T6 | 45 | 516 |
| 126 | g3804916 | 6 | 436 |
| 126 | g3254781 | 1 | 379 |
| 127 | 3504571H1 | 910 | 1216 |
| 127 | g2055741 | 1038 | 1358 |
| 127 | g1270278 | 1025 | 1374 |
| 127 | 2733544H1 | 1137 | 1405 |
| 127 | g1162686 | 1144 | 1490 |
| 127 | g1109059 | 1197 | 1484 |
| 127 | g1774715 | 1212 | 1519 |
| 127 | g3897241 | 1316 | 1718 |
| 127 | 5854467H1 | 1401 | 1557 |
| 127 | g1898243 | 1492 | 1691 |
| 128 | g2358498 | 616 | 997 |
| 128 | 183176H1 | 747 | 971 |
| 128 | 183176R6 | 507 | 971 |
| 128 | 183176R1 | 359 | 971 |
| 128 | 2733388H1 | 659 | 888 |
| 128 | 5616358H1 | 602 | 878 |
| 128 | g2824012 | 433 | 792 |
| 128 | g4762579 | 167 | 600 |
| 128 | 7104793H1 | 1 | 520 |
| 128 | 4004284H1 | 207 | 474 |
| 129 | 3928775H1 | 1 | 192 |
| 129 | 2562126T6 | 40 | 182 |
| 130 | g4902006 | 709 | 895 |
| 130 | 2562126T6 | 708 | 852 |
| 130 | 839936R1 | 828 | 1385 |
| 130 | 839936H1 | 828 | 1073 |
| 130 | 1685280F6 | 1040 | 1477 |
| 130 | 3869224H1 | 1001 | 1301 |
| 130 | 4032140H1 | 1443 | 1700 |
| 130 | 1597096H1 | 1442 | 1628 |
| 130 | 3438058T6 | 1483 | 1957 |
| 130 | 4032240T9 | 1491 | 1900 |
| 130 | g3804916 | 1564 | 1997 |
| 130 | g3432508 | 1574 | 2002 |
| 130 | g3254781 | 1621 | 2002 |
| 130 | 1947742T6 | 689 | 861 |
| 130 | g928730 | 693 | 892 |
| 130 | 3223525H1 | 1152 | 1474 |
| 130 | 5259815H1 | 1 | 206 |
| 130 | 3568526H1 | 71 | 366 |
| 130 | 1289824F6 | 181 | 737 |
| 130 | 1289824H1 | 181 | 349 |
| 130 | g2540596 | 600 | 900 |
| 130 | 4216695H1 | 1421 | 1676 |
| 130 | 3438058F6 | 1437 | 1733 |
| 130 | 3438058H1 | 1437 | 1682 |
| 130 | 1597096F6 | 1442 | 2036 |
| 130 | 1685280H1 | 1243 | 1477 |
| 130 | 5028090H1 | 1348 | 1616 |
| 130 | 7213258H1 | 1225 | 1803 |
| 130 | g1395923 | 1213 | 1549 |
| 130 | 3928775H1 | 696 | 891 |
| 130 | 1289824T6 | 249 | 855 |
| 130 | 3843717H1 | 1153 | 1448 |
| 130 | 764159H1 | 693 | 852 |
| 130 | 5724304H1 | 664 | 1213 |
| 130 | 6040888H1 | 665 | 891 |
| 130 | g1225270 | 689 | 892 |
| 130 | 7153412H1 | 656 | 1189 |
| 130 | 3640283T9 | 1701 | 1932 |
| 130 | 3640283T8 | 1701 | 1908 |
| 130 | g3538751 | 1690 | 2005 |
| 130 | 3640283F8 | 1701 | 1949 |
| 130 | 6620992H1 | 717 | 1277 |
| 131 | 3404480H1 | 1921 | 2103 |
| 131 | 5665320H1 | 2177 | 2358 |
| 131 | 872922H1 | 7624 | 7881 |
| 131 | 6881955J1 | 2681 | 3266 |
| 131 | 2825680F6 | 7992 | 8265 |
| 131 | g5036098 | 8059 | 8265 |
| 131 | 4027974T6 | 6903 | 7418 |
| 131 | 5717756H1 | 6914 | 7382 |
| 131 | 2717848T6 | 6942 | 7421 |
| 131 | 4722802H1 | 6965 | 7069 |
| 131 | g3594812 | 7015 | 7468 |
| 131 | 6403421H1 | 7053 | 7317 |
| 131 | g3163456 | 7067 | 7469 |
| 131 | 2649733F6 | 7123 | 7465 |
| 131 | 2649733H1 | 7123 | 7369 |
| 131 | 7003170H1 | 7124 | 7465 |
| 131 | g683322 | 7127 | 7465 |
| 131 | 2649733T6 | 7128 | 7424 |
| 131 | 2484677H1 | 7142 | 7369 |
| 131 | g6075627 | 7147 | 7469 |
| 131 | g3048962 | 7149 | 7468 |
| 131 | 212391H1 | 7200 | 7437 |
| 131 | g6506945 | 7080 | 7465 |
| 131 | g6073337 | 7082 | 7469 |
| 131 | g2630574 | 7111 | 7470 |
| 131 | 5207126H2 | 2813 | 2981 |
| 131 | 2825680H1 | 7994 | 8265 |
| 131 | g560960 | 8006 | 8268 |
| 131 | g796025 | 8011 | 8277 |
| 131 | 1415181H1 | 8034 | 8265 |
| 131 | 3336518H1 | 3434 | 3669 |
| 131 | 4723427H1 | 3458 | 3606 |
| 131 | 70390598D1 | 3509 | 4085 |
| 131 | 264846H1 | 2524 | 2856 |
| 131 | 6357277H1 | 2531 | 2839 |
| 131 | 5544294H1 | 2708 | 2828 |
| 131 | 2101112T6 | 7755 | 8220 |
| 131 | 7162079H1 | 218 | 671 |
| 131 | g2599501 | 1 | 4447 |
| 131 | 6044517J1 | 7709 | 8237 |
| 131 | 810518H1 | 8140 | 8262 |
| 131 | 2101112H1 | 7763 | 8008 |
| 131 | 2101112R6 | 7764 | 8137 |
| 131 | 6559424H1 | 7774 | 8284 |
| 131 | g2140984 | 7774 | 8165 |
| 131 | 3781662H1 | 7815 | 8126 |
| 131 | 744157H1 | 7818 | 8046 |
| 131 | 4774224T9 | 7821 | 8181 |
| 131 | g2324704 | 7831 | 8266 |
| 131 | g3796940 | 7834 | 8265 |
| 131 | g5839127 | 7843 | 8265 |
| 131 | g3932489 | 7870 | 8265 |
| 131 | g683058 | 7964 | 8268 |
| 131 | g1225252 | 7984 | 8268 |
| 131 | 2825680T6 | 7985 | 8226 |
| 131 | 3278368H1 | 7719 | 7959 |
| 131 | 4726587H1 | 7619 | 7844 |
| 131 | 2108075H1 | 7405 | 7654 |
| 131 | g1241115 | 7445 | 7723 |
| 131 | 4632120H1 | 7488 | 7765 |
| 131 | 4632247H1 | 7488 | 7765 |
| 131 | 7154510H1 | 7499 | 7643 |
| 131 | 6162751H1 | 7497 | 8049 |
| 131 | 1593082F6 | 3583 | 4007 |
| 131 | 1593043H1 | 3583 | 3803 |
| 131 | 1593082H1 | 3583 | 3803 |
| 131 | 7236349H1 | 3664 | 4161 |
| 131 | 4004354H1 | 3665 | 3798 |
| 131 | 439020H1 | 3680 | 3902 |
| 131 | 3480603H1 | 3698 | 3865 |
| 131 | g1678362 | 3734 | 3890 |
| 131 | g4691014 | 3746 | 4204 |
| 131 | g6132554 | 3779 | 4207 |
| 131 | g3756265 | 3780 | 4206 |
| 131 | g1199039 | 3835 | 4151 |
| 131 | g819518 | 3859 | 4212 |
| 131 | 2155889F6 | 3867 | 4265 |
| 131 | 2155889H1 | 3867 | 4106 |
| 131 | 3513325H1 | 3880 | 4124 |
| 131 | 70391721D1 | 3896 | 4321 |
| 131 | 2155889T6 | 3964 | 4405 |
| 131 | g5675561 | 3974 | 4452 |
| 131 | 6618788H1 | 3991 | 4472 |
| 131 | g3144228 | 4048 | 4452 |
| 131 | g2751511 | 4080 | 4441 |
| 131 | 1863153F6 | 4273 | 4666 |
| 131 | 1863161F6 | 4273 | 4526 |
| 131 | 1863153H1 | 4273 | 4520 |
| 131 | 385245H1 | 4275 | 4498 |
| 131 | g4970402 | 4364 | 4819 |
| 131 | 5857654H1 | 4368 | 4636 |
| 131 | g1624652 | 4395 | 4466 |
| 131 | g1023422 | 4479 | 4773 |
| 131 | g1023318 | 4511 | 4801 |
| 131 | 3341807F6 | 4527 | 4888 |
| 131 | 3341807H1 | 4527 | 4766 |
| 131 | 4027974F6 | 4592 | 4881 |
| 131 | 4027926H1 | 4592 | 4822 |
| 131 | 6024929H1 | 4601 | 4923 |
| 131 | 2203255H1 | 4755 | 5006 |
| 131 | 4184071H1 | 4839 | 5084 |
| 131 | 3322055F6 | 4994 | 5518 |
| 131 | 3322055H1 | 4994 | 5271 |
| 131 | 1863161T6 | 5057 | 5459 |
| 131 | 811828H1 | 5105 | 5352 |
| 131 | 789137H1 | 5167 | 5226 |
| 131 | g3077295 | 5179 | 5635 |
| 131 | 3242918H1 | 5346 | 5610 |
| 131 | 4578433H1 | 5545 | 5787 |
| 131 | 5694130H1 | 5702 | 5886 |
| 131 | 4255728H1 | 5783 | 6052 |
| 131 | 5673677H1 | 5815 | 5974 |
| 131 | g1880292 | 5855 | 6068 |
| 131 | 5768386H1 | 5903 | 6448 |
| 131 | g1442274 | 5974 | 6178 |
| 131 | g1678263 | 6029 | 6205 |
| 131 | 3607889H1 | 6044 | 6335 |
| 131 | g709099 | 6063 | 6392 |
| 131 | g769480 | 6063 | 6274 |
| 131 | g692094 | 6065 | 6419 |
| 131 | 5036970H1 | 7696 | 7961 |
| 131 | g2212423 | 7200 | 7465 |
| 131 | 3282024T6 | 7255 | 7409 |
| 131 | 3323350H1 | 7271 | 7524 |
| 131 | g2768029 | 7301 | 7465 |
| 131 | 3322677H1 | 7313 | 7591 |
| 131 | g1515911 | 7317 | 7464 |
| 131 | 3865622H1 | 7330 | 7526 |
| 131 | g317850 | 7332 | 7592 |
| 131 | g3174898 | 7357 | 7469 |
| 131 | 4253321H1 | 7391 | 7470 |
| 131 | 4244366H1 | 7390 | 7465 |
| 131 | 3334656H1 | 7394 | 7606 |
| 131 | 6355568H1 | 3040 | 3236 |
| 131 | g1881143 | 3040 | 3347 |
| 131 | 1372008H1 | 7499 | 7667 |
| 131 | 2292865H1 | 7499 | 7721 |
| 131 | g915845 | 7561 | 7784 |
| 131 | 2289132H1 | 7571 | 7802 |
| 131 | 5832896H1 | 7593 | 7874 |
| 131 | 4774272H1 | 7612 | 7881 |
| 131 | 1393295H1 | 7499 | 7692 |
| 131 | 1772773H1 | 7522 | 7784 |
| 131 | 6269862H1 | 6136 | 6658 |
| 131 | g1975380 | 6238 | 6568 |
| 131 | 3389337H1 | 6336 | 6619 |
| 131 | 6706684H1 | 6370 | 6913 |
| 131 | 3282024F6 | 6416 | 6946 |
| 131 | 3282024H1 | 6416 | 6662 |
| 131 | 3854583H1 | 6428 | 6705 |
| 131 | 2509302H1 | 6442 | 6670 |
| 131 | 7225908H1 | 6547 | 7110 |
| 131 | 3147875H1 | 6581 | 6852 |
| 131 | 3797563H1 | 6583 | 6873 |
| 131 | g2398342 | 1058 | 1351 |
| 131 | 6881955H1 | 1921 | 2185 |
| 131 | 2717848F6 | 6584 | 7049 |
| 131 | 2717848H1 | 6584 | 6832 |
| 131 | 4058694H1 | 6629 | 6895 |
| 131 | 025240H1 | 6665 | 6932 |
| 131 | 4270965H1 | 6782 | 6955 |
| 131 | 3341807T6 | 6814 | 7417 |
| 131 | 3322055T6 | 6820 | 7418 |
| 131 | 4027974T9 | 6822 | 7361 |
| 131 | g691854 | 6874 | 7235 |
| 131 | 4029613H1 | 6873 | 7102 |
| 131 | g708808 | 6874 | 7171 |
| 131 | g565754 | 6874 | 7098 |
| 131 | 124772H1 | 6888 | 7030 |
| 132 | 1630826T6 | 1136 | 1410 |
| 132 | g5813217 | 1172 | 1255 |
| 132 | 1631555T6 | 1194 | 1410 |
| 132 | 3878121H1 | 1210 | 1271 |
| 132 | 1986029H1 | 1210 | 1281 |
| 132 | 2956806H1 | 1210 | 1284 |
| 132 | 1372584H1 | 1214 | 1417 |
| 132 | 854216H1 | 1 | 247 |
| 132 | 854312R6 | 1 | 445 |
| 132 | 854312H1 | 1 | 151 |
| 132 | 2465049H1 | 150 | 358 |
| 132 | 2465049F6 | 150 | 697 |
| 132 | 2271395H1 | 192 | 455 |
| 132 | 2271395R6 | 192 | 704 |
| 132 | g2184123 | 196 | 582 |
| 132 | g1692736 | 348 | 740 |
| 132 | 2465049T6 | 519 | 1047 |
| 132 | 1601994H1 | 535 | 677 |
| 132 | 5274102T6 | 569 | 1060 |
| 132 | g3413112 | 652 | 1088 |
| 132 | g1692707 | 687 | 1094 |
| 132 | g3428202 | 689 | 1088 |
| 132 | g1686359 | 836 | 967 |
| 132 | g2714128 | 860 | 1089 |
| 132 | g3843958 | 950 | 1089 |
| 132 | g4394372 | 950 | 1088 |
| 132 | g3844127 | 950 | 1089 |
| 132 | g661412 | 957 | 1252 |
| 133 | g5438299 | 717 | 1143 |
| 133 | g5109774 | 443 | 863 |
| 133 | g5526440 | 420 | 841 |
| 133 | g4599202 | 303 | 725 |
| 133 | 6306770H1 | 1 | 450 |
| 134 | 4186114H1 | 683 | 1024 |
| 134 | 4186114F6 | 683 | 1091 |
| 134 | 4186114T6 | 921 | 1587 |
| 134 | 4165023H1 | 674 | 955 |
| 135 | 2268189H1 | 1 | 231 |
| 135 | 2268189R6 | 1 | 379 |
| 135 | 1470335H1 | 37 | 230 |
| 135 | g2505442 | 142 | 353 |
| 135 | g2505398 | 157 | 521 |
| 135 | g2458763 | 157 | 579 |
| 135 | 1445774H1 | 262 | 507 |
| 135 | 2268189T6 | 334 | 885 |
| 135 | 368103H1 | 520 | 763 |
| 135 | g2328398 | 608 | 929 |
| 135 | 2322571H1 | 690 | 923 |
| 136 | 6114201H1 | 1 | 292 |
| 136 | 3287273H1 | 260 | 505 |
| 136 | g779441 | 375 | 522 |
| 136 | g879517 | 78 | 440 |
| 136 | g870148 | 135 | 450 |
| 136 | g883117 | 77 | 465 |
| 136 | 2120815H1 | 450 | 565 |
| 136 | 1631555H1 | 450 | 565 |
| 136 | 1631555F6 | 450 | 565 |
| 136 | g705961 | 450 | 565 |
| 136 | 1547031H1 | 452 | 565 |
| 136 | 4796579H1 | 455 | 563 |
| 136 | 5081193H1 | 457 | 508 |
| 136 | 1630826F6 | 450 | 565 |
| 136 | 1630826H1 | 398 | 546 |
| 136 | g2023471 | 491 | 696 |
| 137 | 6864812H1 | 1 | 502 |
| 137 | 292419H1 | 194 | 469 |
| 137 | 292419R6 | 196 | 486 |
| 137 | 3960046H2 | 333 | 473 |
| 137 | 3960046F8 | 333 | 926 |
| 137 | g4114677 | 609 | 1055 |
| 137 | g3674688 | 744 | 1053 |
| 137 | 2705604T6 | 744 | 1011 |
| 137 | g3429159 | 744 | 1052 |
| 137 | 1871586H1 | 744 | 869 |
| 137 | 1998855H1 | 744 | 926 |
| 137 | 2135293H1 | 763 | 1036 |
| 137 | 2135293F6 | 763 | 1227 |
| 137 | 2100630H1 | 865 | 1055 |
| 137 | 292419T6 | 874 | 1001 |
| 137 | 3960046T8 | 887 | 1009 |
| 138 | 6100950H1 | 1 | 213 |
| 138 | 6302544H1 | 151 | 468 |
| 138 | 6144369H1 | 166 | 755 |
| 138 | 6144369F8 | 166 | 749 |
| 138 | 6099148H1 | 208 | 488 |
| 138 | 6281896H1 | 383 | 528 |
| 138 | 6111359H1 | 570 | 864 |
| 138 | 6028709H1 | 650 | 932 |
| 138 | 6144369T8 | 846 | 1259 |
| 139 | 5056523H1 | 2466 | 2604 |
| 139 | 2271032H1 | 893 | 1152 |
| 139 | 4243388H1 | 919 | 1251 |
| 139 | 4552488H1 | 958 | 1120 |
| 139 | g1953334 | 885 | 1086 |
| 139 | g1953371 | 885 | 986 |
| 139 | g713204 | 1029 | 1191 |
| 139 | g1967839 | 1091 | 1429 |
| 139 | 3737077H1 | 1295 | 1453 |
| 139 | 2271032R6 | 893 | 1370 |
| 139 | 6326476H1 | 1313 | 1610 |
| 139 | 1845337T6 | 2212 | 2566 |
| 139 | 1845337R6 | 2212 | 2562 |
| 139 | 1845337H1 | 2212 | 2433 |
| 139 | g4288591 | 2215 | 2599 |
| 139 | g5659132 | 2219 | 2551 |
| 139 | 913114H1 | 2228 | 2509 |
| 139 | g1960980 | 2253 | 2604 |
| 139 | g5887689 | 2244 | 2601 |
| 139 | 911966H1 | 2249 | 2503 |
| 139 | g5838009 | 2250 | 2611 |
| 139 | g5913083 | 2251 | 2604 |
| 139 | g3741346 | 2254 | 2611 |
| 139 | g3785022 | 2297 | 2602 |
| 139 | g2463906 | 2312 | 2607 |
| 139 | g4312734 | 2347 | 2599 |
| 139 | g2100983 | 2358 | 2591 |
| 139 | 4519262H1 | 2360 | 2599 |
| 139 | 1272843T6 | 2368 | 2562 |
| 139 | 6104909H1 | 2413 | 2604 |
| 139 | 4180584H1 | 1872 | 2122 |
| 139 | g1968950 | 1886 | 2368 |
| 139 | 5038396H1 | 1888 | 2146 |
| 139 | 6576957H1 | 1907 | 2455 |
| 139 | 3254138H1 | 1908 | 2115 |
| 139 | 5946104H1 | 1910 | 2145 |
| 139 | 2700155F6 | 1922 | 2433 |
| 139 | 2700155H1 | 1922 | 2121 |
| 139 | 2061567T6 | 1951 | 2554 |
| 139 | 3365965H1 | 1983 | 2108 |
| 139 | g5591869 | 2006 | 2220 |
| 139 | 1749882T6 | 2039 | 2571 |
| 139 | 1752020H1 | 2049 | 2258 |
| 139 | 2700155T6 | 2063 | 2563 |
| 139 | 1889107T6 | 2099 | 2562 |
| 139 | 3236592H2 | 2125 | 2340 |
| 139 | 5291302H1 | 2152 | 2398 |
| 139 | g5038163 | 2168 | 2608 |
| 139 | g2355286 | 2200 | 2589 |
| 139 | g3146568 | 2200 | 2604 |
| 139 | 5307668H1 | 1567 | 1691 |
| 139 | g1968949 | 1345 | 1812 |
| 139 | 5840503H2 | 1578 | 1835 |
| 139 | g2229641 | 1360 | 1792 |
| 139 | 6725031H1 | 1675 | 2299 |
| 139 | 476160H1 | 1389 | 1655 |
| 139 | 2061567R6 | 1698 | 2177 |
| 139 | 2061567H1 | 1698 | 1970 |
| 139 | 6158833H1 | 1433 | 1523 |
| 139 | 794690H1 | 1496 | 1698 |
| 139 | 6576416H1 | 1513 | 1850 |
| 139 | 1889107H1 | 1542 | 1812 |
| 139 | 2250985H1 | 1734 | 1964 |
| 139 | 1889107F6 | 1542 | 1892 |
| 139 | 4399135H1 | 1734 | 1991 |
| 139 | 4399331H1 | 1734 | 2000 |
| 139 | 1501428H1 | 1737 | 1930 |
| 139 | 6823952J1 | 1549 | 2162 |
| 139 | 6018675H1 | 1812 | 2378 |
| 139 | 4516041H1 | 1817 | 2066 |
| 139 | 1272843H1 | 1 | 245 |
| 139 | 1272843F6 | 1 | 593 |
| 139 | 1272843F1 | 1 | 331 |
| 139 | 1274319H1 | 21 | 218 |
| 139 | 2598183H1 | 24 | 263 |
| 139 | 3256051H1 | 25 | 268 |
| 139 | 6826541J1 | 33 | 663 |
| 139 | 6823903H1 | 34 | 188 |
| 139 | 6820526H1 | 422 | 747 |
| 139 | 6820526J1 | 422 | 747 |
| 139 | 6826541H1 | 672 | 1240 |
| 139 | 3857549H1 | 797 | 1106 |
| 139 | 1749882F6 | 811 | 1171 |
| 139 | 1749882H1 | 811 | 1085 |
| 139 | 6829715H1 | 860 | 1357 |
| 140 | 1272843F1 | 1 | 331 |
| 140 | 1272843F6 | 1 | 587 |
| 140 | 1272843H1 | 1 | 245 |
| 140 | 1274319H1 | 21 | 218 |
| 140 | 2598183H1 | 24 | 263 |
| 140 | 3256051H1 | 25 | 268 |
| 140 | 3857549H1 | 306 | 615 |
| 140 | 1749882F6 | 320 | 680 |
| 140 | 1749882H1 | 320 | 594 |
| 140 | g1953334 | 394 | 595 |
| 140 | g1953371 | 394 | 495 |
| 140 | 2271032R6 | 402 | 880 |
| 140 | 2271032H1 | 402 | 661 |
| 140 | 4243388H1 | 428 | 761 |
| 140 | 4552488H1 | 467 | 629 |
| 140 | g713204 | 538 | 701 |
| 140 | g1967839 | 600 | 939 |
| 140 | 3737077H1 | 805 | 963 |
| 140 | 6326476H1 | 823 | 1120 |
| 140 | g1968949 | 855 | 1322 |
| 140 | g2229641 | 870 | 1302 |
| 140 | 476160H1 | 899 | 1165 |
| 140 | 6158833H1 | 943 | 1033 |
| 140 | 794690H1 | 1006 | 1208 |
| 140 | 1889107F6 | 1052 | 1402 |
| 140 | 1889107H1 | 1052 | 1322 |
| 140 | 5840503H2 | 1088 | 1345 |
| 140 | 2061567H1 | 1208 | 1480 |
| 140 | 2061567R6 | 1208 | 1687 |
| 140 | 4399331H1 | 1244 | 1510 |
| 140 | 4399135H1 | 1244 | 1501 |
| 140 | 2250985H1 | 1244 | 1474 |
| 140 | 1501428H1 | 1247 | 1440 |
| 140 | 4516041H1 | 1327 | 1576 |
| 140 | 4180584H1 | 1382 | 1632 |
| 140 | g1968950 | 1396 | 1878 |
| 140 | 5038396H1 | 1398 | 1656 |
| 140 | 3254138H1 | 1418 | 1625 |
| 140 | 5946104H1 | 1420 | 1655 |
| 140 | 2700155F6 | 1432 | 1943 |
| 140 | 2700155H1 | 1432 | 1631 |
| 140 | 2061567T6 | 1461 | 2064 |
| 140 | 3365965H1 | 1493 | 1618 |
| 140 | 1749882T6 | 1549 | 2081 |
| 140 | 70366789D1 | 1569 | 1883 |
| 140 | 1752020H1 | 1559 | 1768 |
| 140 | 2700155T6 | 1573 | 2073 |
| 140 | 1889107T6 | 1609 | 2072 |
| 140 | 3236592H2 | 1635 | 1850 |
| 140 | 5291302H1 | 1662 | 1908 |
| 140 | g2355286 | 1710 | 2099 |
| 140 | g3146568 | 1710 | 2114 |
| 140 | 1845337R6 | 1722 | 2072 |
| 140 | 1845337T6 | 1722 | 2076 |
| 140 | 1845337H1 | 1722 | 1943 |
| 140 | g4288591 | 1725 | 2109 |
| 140 | 913114H1 | 1738 | 2019 |
| 140 | g1960980 | 1763 | 2114 |
| 140 | 911966H1 | 1759 | 2013 |
| 140 | g3741346 | 1764 | 2121 |
| 140 | g3785022 | 1807 | 2112 |
| 140 | g2463906 | 1822 | 2117 |
| 140 | g4312734 | 1857 | 2109 |
| 140 | g2100983 | 1868 | 2101 |
| 140 | 4519262H1 | 1870 | 2109 |
| 140 | 1272843T6 | 1878 | 2072 |
| 140 | 6104909H1 | 1923 | 2114 |
| 140 | 5056523H1 | 1976 | 2114 |
| 141 | 2211487F6 | 1 | 511 |
| 141 | 2211487T6 | 195 | 778 |
| 141 | 2412148H1 | 263 | 497 |
| 141 | 2564905H1 | 540 | 809 |
| 141 | 125647H1 | 559 | 768 |
| 141 | 2242295T6 | 625 | 773 |
| 141 | 2211487H1 | 1 | 241 |
| 142 | 3404890H1 | 1 | 218 |
| 142 | 3387263H1 | 44 | 343 |
| 142 | g1225131 | 45 | 275 |
| 142 | 2745513H1 | 52 | 296 |
| 143 | 3330364H1 | 1 | 264 |
| 143 | 2532555H1 | 1 | 245 |
| 143 | 2532555F6 | 1 | 202 |
| 143 | 3452732H1 | 7 | 223 |
| 143 | 6206478H1 | 8 | 551 |
| 143 | 2240790H1 | 7 | 246 |
| 143 | 2240790F6 | 7 | 455 |
| 143 | 4052530H1 | 9 | 302 |
| 143 | 2211943H1 | 38 | 294 |
| 143 | 2207141H1 | 1 | 161 |
| 143 | 6338721H1 | 60 | 530 |
| 143 | 3722062H1 | 63 | 355 |
| 143 | 3564688H1 | 78 | 370 |
| 143 | g1390898 | 122 | 415 |
| 143 | 5154455F6 | 261 | 738 |
| 143 | 5154455H1 | 489 | 738 |
| 143 | 5155056H1 | 492 | 738 |
| 143 | 5440206H1 | 376 | 645 |
| 143 | 6206972H1 | 638 | 1158 |
| 143 | 2240790T6 | 39 | 495 |
| 143 | g2155919 | 45 | 468 |
| 143 | 3219860T6 | 39 | 451 |
| 143 | 3219860H1 | 135 | 451 |
| 143 | 3219860R6 | 1 | 451 |
| 143 | 3221716H1 | 149 | 451 |
| 143 | 2412827H1 | 233 | 450 |
| 143 | g3932128 | 5 | 420 |
| 143 | g2155881 | 1 | 335 |
| 143 | g2775288 | 1 | 329 |
| 143 | g3277838 | 1 | 325 |
| 143 | g1390787 | 5 | 313 |
| 143 | 2532555T6 | 50 | 283 |
| 143 | g1422648 | 1 | 217 |
| 143 | g1783871 | 2 | 194 |
| 144 | 60210582U1 | 1 | 374 |
| 144 | g857093 | 1 | 226 |
| 144 | 60124509B1 | 198 | 336 |
| 145 | g2324899 | 1 | 329 |
| 145 | g4525985 | 12 | 329 |
| 145 | 3516332H1 | 36 | 278 |
| 145 | 3012696F6 | 162 | 329 |
| 145 | 3012696H1 | 162 | 337 |
| 145 | 3012696T6 | 169 | 288 |
| 145 | 561748H1 | 231 | 329 |
| 146 | 6623203J1 | 1 | 573 |
| 146 | 3538176T6 | 590 | 927 |
| 147 | 5643301H1 | 1 | 266 |
| 147 | 5643301F6 | 1 | 412 |
| 147 | 7007819H1 | 12 | 557 |
| 147 | 7008530H1 | 19 | 463 |
| 147 | 7161666H1 | 34 | 435 |
| 147 | 7017571H1 | 42 | 211 |
| 147 | 3277305H1 | 128 | 393 |
| 147 | 3565182H1 | 492 | 782 |
| 147 | g4293857 | 672 | 946 |
| 147 | 2992317H1 | 816 | 1097 |
| 147 | 5643301R6 | 823 | 1114 |
| 147 | 5643301R8 | 939 | 1115 |
| 148 | g3742856 | 2099 | 2440 |
| 148 | g1903217 | 1 | 2439 |
| 148 | 7183853H1 | 1 | 319 |
| 148 | g3665358 | 2048 | 2440 |
| 148 | 6282319H1 | 2330 | 2439 |
| 148 | g3931095 | 2387 | 2439 |
| 148 | g1487041 | 2284 | 2448 |
| 148 | g1365146 | 1897 | 2439 |
| 148 | g1487088 | 1978 | 2188 |
| 148 | 7183071H1 | 2 | 532 |
| 148 | 7184162H1 | 6 | 440 |
| 148 | 7182312H1 | 265 | 747 |
| 148 | g1365200 | 1875 | 2451 |
| 149 | 6807937J1 | 1 | 617 |
| 149 | 934453R6 | 169 | 650 |
| 149 | 934453T6 | 169 | 578 |
| 149 | 934453H1 | 169 | 430 |
| 150 | g2356671 | 1 | 436 |
| 150 | 3878737H1 | 255 | 511 |
| 151 | 3613937H1 | 1 | 286 |
| 151 | 3613937F6 | 1 | 551 |
| 151 | 3356375H1 | 14 | 272 |
| 151 | 4365653H1 | 26 | 279 |
| 151 | 3133682F6 | 168 | 468 |
| 151 | 3133682H1 | 168 | 446 |
| 151 | 2784431H2 | 322 | 410 |
| 151 | 4760428H1 | 378 | 661 |
| 151 | g1987179 | 426 | 713 |
| 151 | 3985930H1 | 427 | 714 |
| 151 | g3840682 | 469 | 880 |
| 151 | 6619463H1 | 506 | 1081 |
| 151 | g3594561 | 524 | 876 |
| 151 | 3613937T6 | 562 | 835 |
| 151 | 6148958H1 | 732 | 1299 |
| 151 | 5067091H1 | 1 | 243 |
| 151 | 2583002H1 | 33 | 298 |
| 151 | 2583002F6 | 33 | 533 |
| 151 | g273554 | 104 | 290 |
| 152 | 70255820V1 | 1 | 485 |
| 152 | 70249242V1 | 70 | 324 |
| 152 | 70254597V1 | 114 | 627 |
| 152 | 70254754V1 | 114 | 607 |
| 152 | 70254688V1 | 137 | 517 |
| 152 | 70255790V1 | 244 | 565 |
| 152 | 70255539V1 | 507 | 565 |
| 152 | 70255201V1 | 514 | 565 |
| 153 | g4194301 | 1 | 458 |
| 153 | g3244448 | 3 | 471 |
| 153 | g3804087 | 5 | 421 |
| 153 | 2383032T6 | 46 | 518 |
| 153 | 70046632V1 | 217 | 736 |
| 153 | 70046986V1 | 102 | 652 |
| 153 | 70047134V1 | 98 | 553 |
| 153 | 6208108H1 | 1 | 540 |
| 153 | 70046575V1 | 96 | 533 |
| 153 | 70047390V1 | 97 | 515 |
| 153 | 2383032F6 | 1 | 492 |
| 153 | 4938245H1 | 119 | 406 |
| 153 | 70047509V1 | 11 | 414 |
| 153 | 2383032H1 | 1 | 228 |
| 153 | 1952540H1 | 1 | 157 |
| 153 | 70046695V1 | 11 | 94 |
| 154 | 750543H1 | 1 | 183 |
| 154 | g1474315 | 42 | 323 |
| 154 | 3722596H1 | 61 | 333 |
| 154 | 4875149F6 | 70 | 195 |
| 154 | 4875149H1 | 70 | 257 |
| 154 | 6561179H1 | 71 | 633 |
| 154 | 2029094H1 | 121 | 372 |
| 154 | 5950630H1 | 125 | 405 |
| 154 | 5950410H1 | 125 | 401 |
| 154 | 4933727H1 | 239 | 384 |
| 154 | 3533254H1 | 273 | 564 |
| 154 | 4875149T6 | 344 | 737 |
| 154 | g3202357 | 457 | 784 |
| 154 | 748032H1 | 574 | 776 |
| 155 | 3751233H1 | 577 | 867 |
| 155 | 5812189H1 | 644 | 840 |
| 155 | 5812190H1 | 644 | 845 |
| 155 | 311752H1 | 657 | 744 |
| 155 | 373638H1 | 663 | 880 |
| 155 | 6333709H1 | 705 | 1240 |
| 155 | 6329688H1 | 705 | 1325 |
| 155 | 7080559H1 | 760 | 1170 |
| 155 | 6532643H1 | 803 | 1384 |
| 155 | 6141236T8 | 989 | 1579 |
| 155 | 4068325T6 | 1018 | 1641 |
| 155 | 7068876H1 | 1069 | 1483 |
| 155 | 3256362H1 | 1308 | 1554 |
| 155 | g1400213 | 1337 | 1682 |
| 155 | g3254782 | 1346 | 1682 |
| 155 | g1383466 | 1395 | 1696 |
| 155 | 6412487H1 | 1494 | 1931 |
| 155 | 5534143H1 | 1578 | 1820 |
| 155 | 6869946H1 | 1717 | 2275 |
| 155 | g4510725 | 1831 | 2272 |
| 155 | 6298042H1 | 1871 | 2146 |
| 155 | g1998847 | 1973 | 2275 |
| 155 | 3541104H1 | 1991 | 2272 |
| 155 | 5499626H1 | 1 | 255 |
| 155 | 5500309H1 | 1 | 227 |
| 155 | 5500026H1 | 1 | 161 |
| 155 | 5499909H1 | 1 | 206 |
| 155 | 2723940H1 | 1 | 243 |
| 155 | 2723940F6 | 1 | 333 |
| 155 | 5643224H1 | 22 | 276 |
| 155 | 3126704H1 | 22 | 298 |
| 155 | g1998848 | 22 | 296 |
| 155 | 3256088H1 | 24 | 259 |
| 155 | 1728945H1 | 26 | 246 |
| 155 | 6141236F8 | 28 | 633 |
| 155 | 6141236H1 | 28 | 370 |
| 155 | 6328822H1 | 47 | 592 |
| 155 | 3388023H1 | 89 | 361 |
| 155 | 6134014H1 | 110 | 401 |
| 155 | 3323733H1 | 192 | 461 |
| 155 | 3409854H1 | 357 | 618 |
| 155 | 4068325F6 | 515 | 1108 |
| 155 | 4068325H1 | 517 | 804 |
| 155 | 3629455H1 | 560 | 835 |
| 156 | 7006557H1 | 1 | 488 |
| 157 | 6459173H2 | 4960 | 5300 |
| 157 | 71034189V1 | 3224 | 3885 |
| 157 | 71033929V1 | 3308 | 3912 |
| 157 | g2166710 | 3325 | 3492 |
| 157 | 71239210V1 | 3365 | 4028 |
| 157 | 71035047V1 | 3406 | 3932 |
| 157 | 71035847V1 | 3407 | 3932 |
| 157 | 71239003V1 | 3407 | 3934 |
| 157 | 7252515J2 | 3410 | 3964 |
| 157 | 617587H1 | 3462 | 3716 |
| 157 | 617587R6 | 3464 | 3954 |
| 157 | 71035452V1 | 3487 | 4142 |
| 157 | 71239589V1 | 3548 | 4177 |
| 157 | 71034494V1 | 3565 | 4148 |
| 157 | 71036467V1 | 3574 | 4249 |
| 157 | 71019079V1 | 3590 | 4079 |
| 157 | 71034628V1 | 3600 | 4175 |
| 157 | 71033566V1 | 3603 | 3776 |
| 157 | 71033563V1 | 3605 | 4306 |
| 157 | 71033740V1 | 3642 | 4142 |
| 157 | 71034052V1 | 3692 | 4322 |
| 157 | 2852347H1 | 3699 | 3792 |
| 157 | 71034523V1 | 3759 | 4400 |
| 157 | 71021626V1 | 3780 | 4091 |
| 157 | 71033276V1 | 3784 | 4394 |
| 157 | 71035439V1 | 3832 | 4224 |
| 157 | 71239794V1 | 3841 | 4296 |
| 157 | 71036851V1 | 3933 | 4532 |
| 157 | 2902436H1 | 4020 | 4325 |
| 157 | 71035620V1 | 4052 | 4596 |
| 157 | 617587T6 | 4094 | 4694 |
| 157 | 71240672V1 | 4243 | 4499 |
| 157 | g2839119 | 4251 | 4718 |
| 157 | 71239718V1 | 4264 | 4729 |
| 157 | 71240638V1 | 4263 | 4415 |
| 157 | g2716554 | 4274 | 4723 |
| 157 | 2839872F6 | 4473 | 4873 |
| 157 | 2839872H2 | 4473 | 4724 |
| 157 | 6701187H1 | 4517 | 5071 |
| 157 | 71033989V1 | 4552 | 4713 |
| 157 | 6700916H1 | 4758 | 5106 |
| 157 | g1353781 | 1 | 3523 |
| 157 | 7183461H1 | 8 | 510 |
| 157 | 5878868F8 | 38 | 589 |
| 157 | 5878868T9 | 38 | 467 |
| 157 | 5878868H1 | 40 | 300 |
| 157 | 6621194J1 | 465 | 1063 |
| 157 | 7044151H1 | 1537 | 2130 |
| 157 | 7115836H2 | 1721 | 2207 |
| 157 | 6891967H1 | 1744 | 2304 |
| 157 | 7112010H2 | 2069 | 2273 |
| 157 | 7115993H2 | 2217 | 2833 |
| 157 | 71036402V1 | 2382 | 2971 |
| 157 | 71036528V1 | 2382 | 2844 |
| 157 | 71036302V1 | 2382 | 2836 |
| 157 | 5682881F6 | 2382 | 2755 |
| 157 | 5682881H1 | 2382 | 2556 |
| 157 | 7121747H1 | 2421 | 3009 |
| 157 | 6554467H1 | 2459 | 2877 |
| 157 | 71035730V1 | 2859 | 3427 |
| 157 | 71034930V1 | 2861 | 3427 |
| 157 | 5693974H1 | 2871 | 3141 |
| 157 | 71033786V1 | 2880 | 3524 |
| 157 | 71239770V1 | 2904 | 3495 |
| 157 | 71036410V1 | 2922 | 3457 |
| 157 | 71036110V1 | 2922 | 3457 |
| 157 | 71036841V1 | 2925 | 3050 |
| 157 | 71238907V1 | 3000 | 3542 |
| 157 | 71033235V1 | 3023 | 3512 |
| 157 | 71239855V1 | 3037 | 3682 |
| 157 | 71035104V1 | 3060 | 3655 |
| 157 | 71240132V1 | 3064 | 3600 |
| 157 | 71022089V1 | 3064 | 3174 |
| 157 | 71020694V1 | 3064 | 3173 |
| 157 | 71033750V1 | 3068 | 3576 |
| 157 | 71036780V1 | 3070 | 3575 |
| 157 | 71240343V1 | 3074 | 3590 |
| 157 | 71035904V1 | 3096 | 3656 |
| 157 | 71239106V1 | 3098 | 3535 |
| 157 | 71239593V1 | 3107 | 3663 |
| 157 | 71036268V1 | 3116 | 3650 |
| 157 | 71033766V1 | 3130 | 3794 |
| 157 | 71036727V1 | 3136 | 3763 |
| 157 | 71036255V1 | 3150 | 3690 |
| 157 | 3246035H1 | 3163 | 3414 |
| 157 | 71236782V1 | 3177 | 3581 |
| 157 | 71034117V1 | 3225 | 3899 |
| 158 | 2702437H1 | 3382 | 3692 |
| 158 | 2028546H1 | 3382 | 3674 |
| 158 | 70469621V1 | 3384 | 3974 |
| 158 | 70477624V1 | 3362 | 3551 |
| 158 | 70464393V1 | 3358 | 3664 |
| 158 | 2028546R6 | 3382 | 3697 |
| 158 | 897912H1 | 3532 | 3695 |
| 158 | 2657383H1 | 3542 | 3682 |
| 158 | 70466432V1 | 3600 | 4154 |
| 158 | 3405032H1 | 2336 | 2592 |
| 158 | 3099417H1 | 2407 | 2687 |
| 158 | 70467835V1 | 2535 | 3117 |
| 158 | 3936618H1 | 2535 | 2799 |
| 158 | 70468637V1 | 2538 | 3232 |
| 158 | 70465732V1 | 3029 | 3708 |
| 158 | g4568142 | 3029 | 3550 |
| 158 | 70477055V1 | 3050 | 3634 |
| 158 | 70479313V1 | 3061 | 3562 |
| 158 | 70480477V1 | 3075 | 3701 |
| 158 | 70480157V1 | 3098 | 3662 |
| 158 | 4564168H1 | 3087 | 3365 |
| 158 | 70466004V1 | 3018 | 3732 |
| 158 | 70478380V1 | 3019 | 3638 |
| 158 | 4557976H1 | 3015 | 3240 |
| 158 | 3618747H1 | 1905 | 2177 |
| 158 | 6510691H1 | 1907 | 2199 |
| 158 | 6510634H1 | 1907 | 2192 |
| 158 | 6510739H1 | 1907 | 2183 |
| 158 | 4554621H1 | 2044 | 2312 |
| 158 | 70464785V1 | 2168 | 2732 |
| 158 | 4554554F6 | 2168 | 2649 |
| 158 | 4554554H1 | 2168 | 2409 |
| 158 | 70466681V1 | 2298 | 2736 |
| 158 | 70467583V1 | 2823 | 3476 |
| 158 | 70480720V1 | 2857 | 3511 |
| 158 | 70479363V1 | 2850 | 3206 |
| 158 | 70476260V1 | 2859 | 3447 |
| 158 | 70481657V1 | 2790 | 3146 |
| 158 | 70466450V1 | 2775 | 3513 |
| 158 | 70476879V1 | 2765 | 3450 |
| 158 | 70477920V1 | 2806 | 3426 |
| 158 | 70466934V1 | 2704 | 3432 |
| 158 | 70465560V1 | 3293 | 3609 |
| 158 | 70476467V1 | 3297 | 3594 |
| 158 | 70479516V1 | 3309 | 3697 |
| 158 | 4792962H1 | 3311 | 3605 |
| 158 | 70467134V1 | 3311 | 3697 |
| 158 | 4554470H1 | 3317 | 3600 |
| 158 | 70466403V1 | 3271 | 3697 |
| 158 | 70480199V1 | 3270 | 3697 |
| 158 | 70469816V1 | 3293 | 3654 |
| 158 | 70468353V1 | 3282 | 3697 |
| 158 | 70481137V1 | 3205 | 3697 |
| 158 | 70466975V1 | 3210 | 3697 |
| 158 | 70466049V1 | 3236 | 3697 |
| 158 | 70478359V1 | 3235 | 3666 |
| 158 | 6515587H1 | 3242 | 3671 |
| 158 | 70478211V1 | 3217 | 3682 |
| 158 | 4558722H1 | 1289 | 1534 |
| 158 | 3618747F6 | 1904 | 2401 |
| 158 | g190751 | 50 | 3497 |
| 158 | g1736195 | 979 | 1234 |
| 158 | 2624295R6 | 1016 | 1412 |
| 158 | 2624295H1 | 1016 | 1225 |
| 158 | 70466944V1 | 3104 | 3692 |
| 158 | 70480113V1 | 3129 | 3556 |
| 158 | 70468718V1 | 3142 | 3695 |
| 158 | 70477732V1 | 3142 | 3772 |
| 158 | 70469007V1 | 3149 | 3697 |
| 158 | 70469207V1 | 3322 | 3657 |
| 158 | 70480324V1 | 3326 | 3682 |
| 158 | 70466503V1 | 3328 | 4068 |
| 158 | 70479148V1 | 3328 | 3697 |
| 158 | 70464268V1 | 3329 | 3697 |
| 158 | 70466743V1 | 3331 | 3487 |
| 158 | 70467082V1 | 3389 | 3682 |
| 158 | 70481575V1 | 3408 | 3682 |
| 158 | 3381962H1 | 3429 | 3697 |
| 158 | 70468987V1 | 3435 | 3682 |
| 158 | 70465226V1 | 3440 | 3697 |
| 158 | 70466253V1 | 2697 | 3264 |
| 158 | 70469171V1 | 2699 | 3374 |
| 158 | 70469220V1 | 2765 | 3324 |
| 158 | 70481022V1 | 2759 | 3486 |
| 158 | 2624295T6 | 2762 | 3351 |
| 158 | 70469934V1 | 2769 | 3329 |
| 158 | 70480048V1 | 2644 | 3023 |
| 158 | 70478224V1 | 2650 | 3136 |
| 158 | 70467878V1 | 2660 | 3107 |
| 158 | 70481284V1 | 2587 | 3175 |
| 158 | 70467582V1 | 2607 | 3072 |
| 158 | 70465805V1 | 2630 | 3318 |
| 158 | 70481125V1 | 2624 | 3464 |
| 158 | 70469427V1 | 2642 | 3286 |
| 158 | 6287762H2 | 1 | 475 |
| 158 | 70478180V1 | 2901 | 3570 |
| 158 | 70465864V1 | 2900 | 3534 |
| 158 | 70479279V1 | 2923 | 3570 |
| 158 | 70476782V1 | 2927 | 3466 |
| 158 | 70469166V1 | 2935 | 3628 |
| 158 | 70476816V1 | 2992 | 3529 |
| 158 | 70480392V1 | 2746 | 3208 |
| 158 | 70478255V1 | 2751 | 3368 |
| 158 | 70478182V1 | 3467 | 4041 |
| 158 | 70498607V1 | 3496 | 3697 |
| 158 | 70465130V1 | 3440 | 3697 |
| 158 | 2301520H1 | 3457 | 3705 |
| 158 | 70467663V1 | 3171 | 3800 |
| 158 | 70479112V1 | 3208 | 3697 |
| 158 | 70467111V1 | 2890 | 3430 |
| 158 | 4562810H1 | 2883 | 3182 |
| 158 | 70473226V1 | 2882 | 3252 |
| 158 | 2620793H1 | 2886 | 3178 |
| 158 | 70464811V1 | 2708 | 3359 |
| 158 | 70481798V1 | 2721 | 3378 |
| 158 | 70478794V1 | 2722 | 3354 |
| 158 | 70467700V1 | 2732 | 3434 |
| 158 | 70481589V1 | 2742 | 3428 |
| 158 | 70466101V1 | 3337 | 3682 |
| 158 | 70468914V1 | 3348 | 3697 |
| 158 | 70479545V1 | 3359 | 3667 |
| 159 | 3874759H1 | 1 | 296 |
| 159 | 3685395H1 | 3 | 271 |
| 159 | 3692640H1 | 4 | 270 |
| 159 | 3692640F6 | 4 | 342 |
| 159 | g3116688 | 4 | 443 |
| 159 | 3445829H2 | 6 | 264 |
| 159 | 306745H1 | 13 | 386 |
| 159 | 945423H1 | 11 | 293 |
| 159 | 988248R1 | 179 | 383 |
| 159 | 988248H1 | 179 | 291 |
| 159 | 4454887H1 | 250 | 519 |
| 159 | 3877090H1 | 350 | 619 |
| 159 | 6901148H1 | 368 | 825 |
| 159 | 3028782F6 | 505 | 841 |
| 159 | 3028782H1 | 505 | 807 |
| 159 | 1005592H1 | 599 | 889 |
| 159 | 4646393H1 | 801 | 1061 |
| 159 | 5278286H1 | 912 | 1125 |
| 159 | 2521969F6 | 950 | 1354 |
| 159 | 983403H1 | 1063 | 1335 |
| 159 | 983403R6 | 1064 | 1315 |
| 159 | 188949H1 | 1106 | 1306 |
| 159 | 983403T6 | 1123 | 1347 |
| 159 | 988925H1 | 1126 | 1349 |
| 159 | 2521969H1 | 1129 | 1354 |
| 159 | 2639257H1 | 1177 | 1339 |
| 159 | 983273H1 | 1241 | 1544 |
| 159 | 3692640T6 | 1247 | 1832 |
| 159 | 4013213H1 | 1308 | 1598 |
| 159 | 3877453H1 | 1331 | 1614 |
| 159 | 4010336H1 | 1328 | 1611 |
| 159 | 2324266H1 | 1337 | 1579 |
| 159 | 3011865F6 | 1340 | 1761 |
| 159 | 3576519H1 | 1339 | 1495 |
| 159 | 5176829H1 | 1348 | 1600 |
| 159 | 3873606H1 | 1351 | 1661 |
| 159 | 4852614H1 | 1363 | 1587 |
| 159 | 3016113F6 | 1376 | 1647 |
| 159 | 3011865H1 | 1376 | 1459 |
| 159 | 058529H1 | 1376 | 1522 |
| 159 | 3958442H1 | 1377 | 1474 |
| 159 | 5276870H1 | 1376 | 1535 |
| 159 | 3578346H1 | 1382 | 1634 |
| 159 | 3016113H1 | 1383 | 1669 |
| 159 | 6337413H1 | 1390 | 1507 |
| 159 | 6338013H1 | 1390 | 1881 |
| 159 | 6335720H1 | 1390 | 1883 |
| 159 | 3688195H1 | 1398 | 1695 |
| 159 | 4151882H1 | 1401 | 1641 |
| 159 | 3874704H1 | 1439 | 1714 |
| 159 | 5169119H1 | 1476 | 1610 |
| 159 | 5278359H1 | 1475 | 1705 |
| 159 | 3685458H1 | 1486 | 1783 |
| 159 | 6332978H1 | 1487 | 1982 |
| 159 | 5531678H1 | 1495 | 1624 |
| 159 | g5395484 | 1513 | 1854 |
| 159 | 3693626H1 | 1512 | 1804 |
| 159 | 4152946H1 | 1520 | 1792 |
| 159 | 3875504H1 | 1549 | 1851 |
| 159 | 3890704H1 | 1548 | 1842 |
| 159 | 4466716H1 | 1582 | 1839 |
| 159 | g395449 | 1591 | 1923 |
| 159 | 5167337H1 | 1592 | 1800 |
| 159 | 920392H1 | 1611 | 1923 |
| 159 | 3011865T6 | 1618 | 1972 |
| 159 | 3016113T6 | 1674 | 2155 |
| 159 | 5277911H1 | 1697 | 1935 |
| 159 | 2636153H1 | 1700 | 1929 |
| 159 | 1567405H1 | 1706 | 1889 |
| 159 | 462513H1 | 1706 | 1931 |
| 159 | 462513R6 | 1706 | 2044 |
| 159 | 3877471H1 | 1721 | 1990 |
| 159 | 5277625H1 | 1723 | 1973 |
| 159 | 3016674H1 | 1739 | 2024 |
| 159 | 3045188H1 | 1739 | 2025 |
| 159 | 977605T1 | 1764 | 2179 |
| 159 | 5299216H1 | 1761 | 2001 |
| 159 | 977605H1 | 1764 | 2075 |
| 159 | 977605R1 | 1764 | 2143 |
| 159 | 3028782T6 | 1767 | 2194 |
| 159 | 3487465H1 | 1765 | 2054 |
| 159 | 462261H1 | 1817 | 2014 |
| 159 | 5170457H1 | 1823 | 2011 |
| 159 | 3683935H1 | 1823 | 2109 |
| 159 | 5803204H1 | 1826 | 2024 |
| 159 | 979691H1 | 1836 | 2120 |
| 159 | g434193 | 1852 | 2026 |
| 159 | 3013979H1 | 1877 | 2169 |
| 159 | 921802H1 | 1888 | 2214 |
| 159 | 920885H1 | 1888 | 2204 |
| 159 | 5278931H1 | 1996 | 2214 |
| 159 | 058768H1 | 1998 | 2201 |
| 159 | 3045908H1 | 2013 | 2281 |
| 159 | g2787073 | 2048 | 2207 |
| 160 | g6086623 | 4494 | 4929 |
| 160 | g2569620 | 4498 | 4936 |
| 160 | g2754538 | 4500 | 4930 |
| 160 | 3022465H1 | 4515 | 4743 |
| 160 | 2448511H1 | 4519 | 4765 |
| 160 | 3760535H1 | 4524 | 4835 |
| 160 | 2640174H1 | 4524 | 4780 |
| 160 | 517085T6 | 4532 | 4894 |
| 160 | 5338680H1 | 4533 | 4719 |
| 160 | 735966R1 | 4536 | 4961 |
| 160 | 735966H1 | 4536 | 4780 |
| 160 | 1592447H1 | 4536 | 4749 |
| 160 | 1591916H1 | 4536 | 4740 |
| 160 | 5022872H1 | 4538 | 4830 |
| 160 | g3253612 | 4546 | 4951 |
| 160 | g3843392 | 4553 | 4929 |
| 160 | g3843398 | 4554 | 4929 |
| 160 | g4080952 | 4558 | 4936 |
| 160 | 3814494H1 | 4561 | 4861 |
| 160 | 2009809H1 | 4568 | 4749 |
| 160 | g1271129 | 4575 | 4962 |
| 160 | g1068131 | 4586 | 4910 |
| 160 | 2096906H1 | 4584 | 4854 |
| 160 | 2603246H1 | 4589 | 4881 |
| 160 | g829798 | 4598 | 4943 |
| 160 | g1068126 | 4598 | 4905 |
| 160 | 2006525H1 | 4597 | 4764 |
| 160 | 628342H1 | 4598 | 4863 |
| 160 | 1943588H1 | 4599 | 4852 |
| 160 | g1044543 | 4602 | 4916 |
| 160 | g777642 | 4610 | 4930 |
| 160 | 2685217H1 | 4607 | 4864 |
| 160 | 1365826H1 | 4615 | 4876 |
| 160 | 1365826R1 | 4615 | 4941 |
| 160 | 1365826T1 | 4615 | 4910 |
| 160 | 6371841H1 | 4621 | 4912 |
| 160 | g1525497 | 4622 | 4933 |
| 160 | 5704503H1 | 4643 | 4918 |
| 160 | 4060183H1 | 4644 | 4927 |
| 160 | 1416579H1 | 4644 | 4915 |
| 160 | 1416547H1 | 4644 | 4908 |
| 160 | 4569760H2 | 4658 | 4921 |
| 160 | 3721940H1 | 4651 | 4962 |
| 160 | g852574 | 4663 | 4906 |
| 160 | g1023637 | 4669 | 4890 |
| 160 | 3712504H1 | 4671 | 4971 |
| 160 | 4825195H1 | 4672 | 4924 |
| 160 | g985536 | 4678 | 4902 |
| 160 | 3568055H1 | 4680 | 4986 |
| 160 | g2819448 | 4690 | 4932 |
| 160 | g1024915 | 4694 | 4924 |
| 160 | g4125247 | 4702 | 4939 |
| 160 | 4254485T8 | 4705 | 4940 |
| 160 | 5714026H1 | 4707 | 4991 |
| 160 | 770816R1 | 4713 | 5202 |
| 160 | 770816H1 | 4713 | 4968 |
| 160 | 1901241H1 | 4719 | 4976 |
| 160 | 1898941H1 | 4719 | 4974 |
| 160 | g2903316 | 4724 | 4936 |
| 160 | 6715734H1 | 4726 | 4923 |
| 160 | 2897118H1 | 4729 | 4947 |
| 160 | 986309R1 | 4747 | 5145 |
| 160 | 986309H1 | 4747 | 5067 |
| 160 | 986309T1 | 4747 | 5241 |
| 160 | g2837150 | 4762 | 5278 |
| 160 | 2027901H1 | 4764 | 5060 |
| 160 | 2654529H1 | 4773 | 4942 |
| 160 | g3057209 | 4773 | 5234 |
| 160 | 5871918H1 | 4775 | 5080 |
| 160 | g3735780 | 4799 | 5287 |
| 160 | g5424599 | 4804 | 5282 |
| 160 | g4598235 | 4806 | 5285 |
| 160 | g4684860 | 4807 | 5285 |
| 160 | g5367615 | 4807 | 5287 |
| 160 | g4437224 | 4810 | 5285 |
| 160 | 4790610H1 | 4810 | 4940 |
| 160 | g3412956 | 4812 | 5285 |
| 160 | 1212364H1 | 4813 | 5002 |
| 160 | g4153588 | 4815 | 5285 |
| 160 | g3050313 | 4816 | 5285 |
| 160 | 4340133H1 | 4817 | 5098 |
| 160 | g4071897 | 4821 | 5290 |
| 160 | g4762931 | 4823 | 5287 |
| 160 | g4393968 | 4823 | 5287 |
| 160 | 6872074H1 | 4828 | 5313 |
| 160 | g3308744 | 4829 | 5293 |
| 160 | g3887256 | 4843 | 5287 |
| 160 | g5811461 | 4843 | 5285 |
| 160 | g4372712 | 4842 | 5285 |
| 160 | g3597624 | 4846 | 5291 |
| 160 | g3869879 | 4848 | 5286 |
| 160 | 4201581H1 | 4848 | 4946 |
| 160 | g4081955 | 4853 | 5290 |
| 160 | g3840562 | 4854 | 5286 |
| 160 | g5809990 | 4854 | 5270 |
| 160 | 4129277H2 | 4854 | 5127 |
| 160 | g5904237 | 4855 | 5278 |
| 160 | g4969558 | 4856 | 5276 |
| 160 | g4292330 | 4857 | 5288 |
| 160 | g5849107 | 4857 | 5291 |
| 160 | 3621721H1 | 4856 | 5149 |
| 160 | g4987769 | 4863 | 4936 |
| 160 | 347302H1 | 4867 | 5086 |
| 160 | 5408358H1 | 4872 | 5129 |
| 160 | g3896761 | 4879 | 5287 |
| 160 | g3279962 | 4892 | 5285 |
| 160 | g4244340 | 4895 | 5285 |
| 160 | g3658851 | 4903 | 5285 |
| 160 | g4983854 | 4906 | 5285 |
| 160 | 5388769H1 | 4911 | 5186 |
| 160 | 6215533H1 | 4912 | 5285 |
| 160 | 5388768H1 | 4911 | 5186 |
| 160 | g2816194 | 4920 | 5285 |
| 160 | g5547017 | 4930 | 5287 |
| 160 | g4089434 | 4932 | 5285 |
| 160 | g1484540 | 4934 | 5285 |
| 160 | 1739863R6 | 4940 | 5237 |
| 160 | 1739863H1 | 4940 | 5170 |
| 160 | 4207604H1 | 4940 | 5116 |
| 160 | 5578672H1 | 4950 | 5211 |
| 160 | 1689363H1 | 4952 | 5138 |
| 160 | 2606160H1 | 4957 | 5207 |
| 160 | 2007185H1 | 4999 | 5202 |
| 160 | g795782 | 5014 | 5285 |
| 160 | 6613821H1 | 5033 | 5165 |
| 160 | 4772611H1 | 5036 | 5285 |
| 160 | 4923819H1 | 5037 | 5285 |
| 160 | g1188096 | 5040 | 5232 |
| 160 | g2540711 | 5047 | 5403 |
| 160 | g2022546 | 5046 | 5285 |
| 160 | 3445038H1 | 5046 | 5257 |
| 160 | g5447532 | 5058 | 5345 |
| 160 | 3486818H1 | 5071 | 5285 |
| 160 | g1479409 | 5084 | 5403 |
| 160 | g1064422 | 5084 | 5269 |
| 160 | g2821180 | 5087 | 5406 |
| 160 | g1543936 | 5098 | 5408 |
| 160 | 2769102H1 | 5102 | 5271 |
| 160 | g815309 | 5131 | 5403 |
| 160 | g4893445 | 5128 | 5293 |
| 160 | 1923809H1 | 5134 | 5289 |
| 160 | g29237 | 5139 | 5403 |
| 160 | g1548590 | 5143 | 5406 |
| 160 | g1202222 | 5146 | 5319 |
| 160 | g3202374 | 5145 | 5288 |
| 160 | g1665338 | 5149 | 5271 |
| 160 | g1228664 | 5151 | 5285 |
| 160 | 7336808H1 | 5161 | 5339 |
| 160 | 2807655H1 | 5176 | 5279 |
| 160 | 1741769R6 | 5216 | 5354 |
| 160 | 1741769H1 | 5216 | 5339 |
| 160 | 1741195H1 | 5216 | 5339 |
| 160 | 1741769T6 | 5216 | 5302 |
| 160 | g810674 | 5258 | 5404 |
| 160 | g944127 | 5261 | 5394 |
| 160 | 4125568H1 | 4959 | 5163 |
| 160 | g6464630 | 4967 | 5403 |
| 160 | g1501886 | 4969 | 5294 |
| 160 | 4537948H1 | 4971 | 5252 |
| 160 | 4699937H1 | 4974 | 5159 |
| 160 | g3038275 | 4981 | 5352 |
| 160 | g1664143 | 4988 | 5339 |
| 160 | 1283369T6 | 4991 | 5295 |
| 160 | 1283369F6 | 4997 | 5350 |
| 160 | 1283369H1 | 4997 | 5277 |
| 160 | 056712H1 | 3755 | 4005 |
| 160 | g829797 | 3765 | 4195 |
| 160 | g852673 | 3767 | 4118 |
| 160 | g775984 | 3767 | 4023 |
| 160 | 2967706H1 | 3805 | 4117 |
| 160 | 2539208H1 | 3812 | 4049 |
| 160 | 2443012H1 | 3817 | 4068 |
| 160 | 988892H1 | 3821 | 4063 |
| 160 | 2291992H1 | 3821 | 4048 |
| 160 | 4178688H1 | 3822 | 4086 |
| 160 | 1833818H1 | 3831 | 4088 |
| 160 | 6368013H1 | 3838 | 4152 |
| 160 | 2134837H1 | 3858 | 4104 |
| 160 | 5657126H1 | 3862 | 4145 |
| 160 | g1665244 | 3871 | 4196 |
| 160 | 2288056H1 | 3873 | 4122 |
| 160 | 3740644H1 | 3903 | 4101 |
| 160 | 4714119H1 | 3904 | 4202 |
| 160 | 5352624H1 | 3913 | 4159 |
| 160 | g1272802 | 3939 | 4460 |
| 160 | 6388078H1 | 3935 | 4238 |
| 160 | 3056718H1 | 3936 | 4205 |
| 160 | g1044542 | 3941 | 4227 |
| 160 | g1525554 | 3942 | 4231 |
| 160 | g1625857 | 3970 | 4363 |
| 160 | g1977647 | 3972 | 4318 |
| 160 | 056202H1 | 3982 | 4165 |
| 160 | 1259638H1 | 4000 | 4255 |
| 160 | 1259479F1 | 4000 | 4527 |
| 160 | 1259638F1 | 4000 | 4492 |
| 160 | 4383783H1 | 4010 | 4274 |
| 160 | g1440113 | 4032 | 4192 |
| 160 | 6856341H1 | 4048 | 4572 |
| 160 | g944630 | 4051 | 4388 |
| 160 | 5950009H1 | 4064 | 4381 |
| 160 | g1240400 | 4066 | 4202 |
| 160 | 5387777H1 | 4076 | 4340 |
| 160 | 4342232H1 | 4088 | 4243 |
| 160 | 3022534H1 | 4093 | 4335 |
| 160 | 293402R6 | 4110 | 4551 |
| 160 | 293402H1 | 4110 | 4227 |
| 160 | 2772843H1 | 4110 | 4346 |
| 160 | 2411009H1 | 4125 | 4343 |
| 160 | 2819785H1 | 4126 | 4424 |
| 160 | 4512962H1 | 4131 | 4407 |
| 160 | g1978183 | 4133 | 4478 |
| 160 | g4152729 | 4145 | 4491 |
| 160 | 1690358H1 | 4144 | 4422 |
| 160 | 6334629H1 | 4149 | 4624 |
| 160 | 5387664H1 | 4149 | 4413 |
| 160 | 6334630H1 | 4149 | 4622 |
| 160 | 4600162H1 | 4153 | 4447 |
| 160 | g5673211 | 4161 | 4640 |
| 160 | 055138H1 | 4167 | 4394 |
| 160 | g4648135 | 4168 | 4628 |
| 160 | 986584H1 | 4169 | 4465 |
| 160 | 986584R1 | 4169 | 4584 |
| 160 | 2290964H1 | 4171 | 4423 |
| 160 | 3056037H1 | 4188 | 4464 |
| 160 | 2784447H2 | 4197 | 4434 |
| 160 | 3567327H1 | 4199 | 4491 |
| 160 | 4135683H1 | 4235 | 4471 |
| 160 | 3789022H1 | 4276 | 4550 |
| 160 | 4833507H1 | 4282 | 4552 |
| 160 | 2285967H1 | 4282 | 4510 |
| 160 | 3469203H1 | 4288 | 4433 |
| 160 | 4573834H1 | 4295 | 4553 |
| 160 | g2006388 | 4295 | 4536 |
| 160 | g810580 | 4327 | 4574 |
| 160 | 4140391H1 | 4331 | 4620 |
| 160 | g2825270 | 4341 | 4709 |
| 160 | g815902 | 4342 | 4600 |
| 160 | g2898761 | 4342 | 4764 |
| 160 | 6601487H1 | 4347 | 4413 |
| 160 | 3812509H1 | 4361 | 4570 |
| 160 | 5605594H1 | 4370 | 4614 |
| 160 | 5401012H1 | 4376 | 4636 |
| 160 | g1023636 | 4377 | 4603 |
| 160 | 056184H1 | 4376 | 4598 |
| 160 | g1060832 | 4378 | 4490 |
| 160 | 3253684H1 | 4392 | 4482 |
| 160 | g1501988 | 4392 | 4873 |
| 160 | 293402T6 | 4396 | 4872 |
| 160 | g2825431 | 4397 | 4710 |
| 160 | 4421168H1 | 4431 | 4671 |
| 160 | 2436027H1 | 4437 | 4685 |
| 160 | g5177561 | 4447 | 4930 |
| 160 | g797106 | 4448 | 4687 |
| 160 | g2874401 | 4448 | 4939 |
| 160 | g3597789 | 4460 | 4933 |
| 160 | 4649987H1 | 4465 | 4747 |
| 160 | 2920060H1 | 4466 | 4748 |
| 160 | g3675776 | 4469 | 4933 |
| 160 | 986389H1 | 4480 | 4623 |
| 160 | 6291011H1 | 4484 | 4747 |
| 160 | g5527815 | 4488 | 4941 |
| 160 | g6086615 | 4491 | 4929 |
| 160 | g1469867 | 1 | 5285 |
| 160 | 7086887H1 | 1 | 516 |
| 160 | 3287254H1 | 8 | 248 |
| 160 | g273451 | 81 | 456 |
| 160 | g1057697 | 186 | 523 |
| 160 | 3903676H1 | 189 | 440 |
| 160 | g434410 | 279 | 603 |
| 160 | 3698379H1 | 455 | 747 |
| 160 | 5643561H1 | 480 | 749 |
| 160 | 4960333H1 | 502 | 761 |
| 160 | g2277376 | 522 | 931 |
| 160 | 5090209H1 | 584 | 831 |
| 160 | 525014H1 | 602 | 841 |
| 160 | 525014R6 | 602 | 845 |
| 160 | 162678H1 | 741 | 981 |
| 160 | 6332784H1 | 1028 | 1600 |
| 160 | 3872857H1 | 1053 | 1308 |
| 160 | 525014T6 | 1109 | 1587 |
| 160 | g395681 | 1123 | 1460 |
| 160 | g6086016 | 1169 | 1629 |
| 160 | g2779861 | 1232 | 1536 |
| 160 | g1057696 | 1305 | 1626 |
| 160 | 4806634H1 | 1367 | 1619 |
| 160 | 5864502H1 | 1415 | 1701 |
| 160 | 156350R6 | 1439 | 1717 |
| 160 | 156350H1 | 1439 | 1616 |
| 160 | 5502785H1 | 1447 | 1672 |
| 160 | 4696595H1 | 1488 | 1665 |
| 160 | g3701554 | 1534 | 1625 |
| 160 | 3825811H1 | 1546 | 1852 |
| 160 | 3825859H1 | 1546 | 1803 |
| 160 | g1880691 | 1643 | 1799 |
| 160 | g691032 | 1722 | 2124 |
| 160 | g573544 | 1722 | 2062 |
| 160 | g708679 | 1722 | 2043 |
| 160 | 6610185H1 | 1732 | 2259 |
| 160 | 1257342H1 | 1775 | 2018 |
| 160 | 2532881H1 | 1859 | 2182 |
| 160 | g3416512 | 1887 | 2323 |
| 160 | 2722041H1 | 1915 | 2155 |
| 160 | 7191660H2 | 2044 | 2659 |
| 160 | 1804357H1 | 2065 | 2340 |
| 160 | 1806920H1 | 2065 | 2330 |
| 160 | 5602907H1 | 2082 | 2351 |
| 160 | 2830493H1 | 2090 | 2352 |
| 160 | 5542070H1 | 2153 | 2364 |
| 160 | 7171003H1 | 2174 | 2627 |
| 160 | 3109807H1 | 2202 | 2486 |
| 160 | 1439519H1 | 2327 | 2604 |
| 160 | 086846H1 | 2351 | 2417 |
| 160 | 6743069H1 | 2379 | 2880 |
| 160 | 5092027H1 | 2401 | 2674 |
| 160 | 3595466H1 | 2452 | 2678 |
| 160 | 3120055H1 | 2600 | 2875 |
| 160 | 4028493H1 | 2623 | 2861 |
| 160 | 6918376H1 | 2649 | 3021 |
| 160 | 833106R1 | 2689 | 3287 |
| 160 | 833106H1 | 2689 | 2941 |
| 160 | g1068214 | 2708 | 3062 |
| 160 | g1068219 | 2719 | 3068 |
| 160 | 386166H1 | 2777 | 3061 |
| 160 | 3163260H1 | 2798 | 3086 |
| 160 | 6490409H1 | 2830 | 3338 |
| 160 | 2122144H1 | 2851 | 3109 |
| 160 | g1484539 | 2902 | 3404 |
| 160 | 3120466H1 | 2924 | 3191 |
| 160 | g1062289 | 2966 | 3116 |
| 160 | 4178517H1 | 3003 | 3265 |
| 160 | 2505067H1 | 3016 | 3259 |
| 160 | 2505367H1 | 3016 | 3245 |
| 160 | 7034903H1 | 3023 | 3630 |
| 160 | g1259693 | 3042 | 3222 |
| 160 | 7315867H1 | 3070 | 3471 |
| 160 | 5370265H1 | 3126 | 3359 |
| 160 | g2022547 | 3176 | 3463 |
| 160 | 2707316H1 | 3197 | 3502 |
| 160 | 2894887H1 | 3197 | 3446 |
| 160 | g796865 | 3229 | 3473 |
| 160 | 3523324H1 | 3238 | 3589 |
| 160 | g1479499 | 3257 | 3560 |
| 160 | g1548639 | 3257 | 3626 |
| 160 | g943258 | 3257 | 3559 |
| 160 | g1067965 | 3257 | 3571 |
| 160 | 5371320H1 | 3264 | 3396 |
| 160 | 4441134H1 | 3378 | 3656 |
| 160 | g682946 | 3380 | 3626 |
| 160 | 2029016H1 | 3406 | 3676 |
| 160 | 5665710H1 | 3409 | 3680 |
| 160 | 5544787H1 | 3408 | 3621 |
| 160 | g3405453 | 3410 | 3838 |
| 160 | g1225697 | 3410 | 3783 |
| 160 | g985312 | 3414 | 3790 |
| 160 | g944631 | 3420 | 3511 |
| 160 | 2471283H1 | 3421 | 3645 |
| 160 | 3561909H1 | 3421 | 3714 |
| 160 | g965314 | 3442 | 3674 |
| 160 | 2748973H1 | 3446 | 3719 |
| 160 | 5621772H1 | 3458 | 3598 |
| 160 | g1064141 | 3477 | 3785 |
| 160 | 7033911H1 | 3483 | 4011 |
| 160 | 517085H1 | 3502 | 3727 |
| 160 | 517085R6 | 3503 | 3982 |
| 160 | 4655472H1 | 3524 | 3710 |
| 160 | 3521721H1 | 3525 | 3865 |
| 160 | 2668464H1 | 3540 | 3800 |
| 160 | 2285765H1 | 3578 | 3795 |
| 160 | 2796870H1 | 3597 | 3846 |
| 160 | 2761118H1 | 3598 | 3859 |
| 160 | 5544423H1 | 3615 | 3699 |
| 160 | 338142H1 | 3622 | 3850 |
| 160 | 4545187H1 | 3627 | 3871 |
| 160 | 2243019H1 | 3628 | 3885 |
| 160 | 2772059H1 | 3629 | 3889 |
| 160 | 2960117H1 | 3636 | 3937 |
| 160 | g1969950 | 3641 | 4014 |
| 160 | g1947603 | 3654 | 3817 |
| 160 | 5002748H1 | 3687 | 3844 |
| 160 | 4062722H1 | 3721 | 4010 |
| 160 | 7107841H1 | 3740 | 4101 |
| 160 | g1024914 | 3749 | 4082 |
| 160 | 5546516H1 | 3750 | 3928 |
| 161 | g1219848 | 2061 | 2389 |
| 161 | 5406570H1 | 2061 | 2269 |
| 161 | 5451646H1 | 2063 | 2243 |
| 161 | g2000931 | 2063 | 2316 |
| 161 | 6517527H1 | 2068 | 2140 |
| 161 | 4426132H1 | 2214 | 2389 |
| 161 | 3468616H1 | 2068 | 2331 |
| 161 | 5451867H1 | 2073 | 2323 |
| 161 | 6441810H1 | 2085 | 2530 |
| 161 | 7076574H1 | 2068 | 2555 |
| 161 | 70346502D1 | 2070 | 2384 |
| 161 | 4772876H1 | 2073 | 2352 |
| 161 | 805234H1 | 2073 | 2269 |
| 161 | 6763871H1 | 2078 | 2569 |
| 161 | 7079082H1 | 2081 | 2565 |
| 161 | 6996593H1 | 2081 | 2575 |
| 161 | 5693746H1 | 2096 | 2319 |
| 161 | 4760746H1 | 2081 | 2371 |
| 161 | 4760754H1 | 2082 | 2371 |
| 161 | 3466673H1 | 2085 | 2180 |
| 161 | 3955495H1 | 2106 | 2385 |
| 161 | 3945275H1 | 2213 | 2490 |
| 161 | 6891449H1 | 2214 | 2316 |
| 161 | 4195061H1 | 2215 | 2518 |
| 161 | 3781801H1 | 2096 | 2379 |
| 161 | 2570710H1 | 2219 | 2467 |
| 161 | 4690839H1 | 2097 | 2352 |
| 161 | g683009 | 2100 | 2389 |
| 161 | 5682533H1 | 2099 | 2378 |
| 161 | 5575001H1 | 2106 | 2253 |
| 161 | 1505114H1 | 2127 | 2344 |
| 161 | 4091605H1 | 2114 | 2422 |
| 161 | 5832027H1 | 2123 | 2377 |
| 161 | 5401121H1 | 2219 | 2429 |
| 161 | 5388353H1 | 2219 | 2494 |
| 161 | 2704568H1 | 2127 | 2350 |
| 161 | 5834524H1 | 2132 | 2405 |
| 161 | 7103141H1 | 2155 | 2265 |
| 161 | 4315855H1 | 2139 | 2389 |
| 161 | 5834443H1 | 2139 | 2343 |
| 161 | 664952H1 | 2142 | 2367 |
| 161 | 1992383H1 | 2143 | 2344 |
| 161 | 3468549H1 | 2219 | 2487 |
| 161 | 4256959H1 | 2161 | 2426 |
| 161 | 6993692H1 | 2148 | 2573 |
| 161 | 534218H1 | 2156 | 2414 |
| 161 | 664384H1 | 2156 | 2421 |
| 161 | 4713170H1 | 2177 | 2420 |
| 161 | 663504H1 | 2219 | 2347 |
| 161 | 2247226H1 | 2184 | 2445 |
| 161 | 2245218H1 | 2184 | 2452 |
| 161 | 531779H1 | 2184 | 2426 |
| 161 | 2042975H1 | 2184 | 2450 |
| 161 | 6741472H1 | 2204 | 2750 |
| 161 | 4490622H1 | 2204 | 2747 |
| 161 | 4063891H1 | 2241 | 2390 |
| 161 | 5960583H1 | 2242 | 2800 |
| 161 | 3781135H1 | 2190 | 2495 |
| 161 | 7071563H1 | 2208 | 2569 |
| 161 | 6891449J1 | 2214 | 2315 |
| 161 | 554022H1 | 2202 | 2438 |
| 161 | 4532533H1 | 2273 | 2435 |
| 161 | 6739828H1 | 2219 | 2805 |
| 161 | 4691246H1 | 2208 | 2424 |
| 161 | 3470773H1 | 2209 | 2454 |
| 161 | 5967249H1 | 2224 | 2744 |
| 161 | 6765073J1 | 2275 | 2692 |
| 161 | 666644H1 | 2212 | 2384 |
| 161 | 5579450H1 | 2212 | 2365 |
| 161 | 1618034H1 | 2212 | 2418 |
| 161 | 7088449H1 | 1 | 552 |
| 161 | 7073146H1 | 170 | 726 |
| 161 | 6784888H1 | 439 | 960 |
| 161 | 6882093H1 | 479 | 990 |
| 161 | 6882161H1 | 479 | 992 |
| 161 | 6766193H1 | 480 | 961 |
| 161 | 6977268H1 | 504 | 925 |
| 161 | 7230035H1 | 585 | 1180 |
| 161 | 7075220H1 | 629 | 1180 |
| 161 | 1485846F6 | 646 | 910 |
| 161 | g1975476 | 664 | 884 |
| 161 | 1486578H1 | 676 | 912 |
| 161 | 5968971H1 | 685 | 1143 |
| 161 | 1476202H1 | 762 | 924 |
| 161 | 865333H1 | 780 | 910 |
| 161 | 7193726H2 | 786 | 1230 |
| 161 | 6891594J1 | 877 | 1507 |
| 161 | 4031012H1 | 981 | 1202 |
| 161 | 6446990H1 | 988 | 1498 |
| 161 | g6133077 | 1024 | 1478 |
| 161 | 3941692H1 | 1056 | 1337 |
| 161 | 4772703H1 | 1105 | 1361 |
| 161 | g668477 | 1117 | 1416 |
| 161 | g870488 | 1118 | 1438 |
| 161 | g698735 | 1117 | 1456 |
| 161 | g669863 | 1117 | 1378 |
| 161 | g768138 | 1143 | 1468 |
| 161 | g1062711 | 1172 | 1485 |
| 161 | g875401 | 1174 | 1475 |
| 161 | 1617044H1 | 1178 | 1392 |
| 161 | 5781411H1 | 1182 | 1459 |
| 161 | 532473H1 | 1193 | 1433 |
| 161 | 3945042H1 | 1194 | 1480 |
| 161 | 4758039H1 | 1193 | 1440 |
| 161 | 3466710H1 | 1203 | 1464 |
| 161 | 5860513H1 | 1221 | 1490 |
| 161 | 6770719H1 | 1238 | 1355 |
| 161 | 5656957H1 | 1238 | 1487 |
| 161 | 5204013H1 | 1238 | 1505 |
| 161 | 4254309H1 | 1257 | 1459 |
| 161 | 5578708H1 | 1259 | 1489 |
| 161 | 5013751H1 | 1264 | 1491 |
| 161 | 4019484H1 | 1278 | 1543 |
| 161 | 6063870H1 | 1281 | 1564 |
| 161 | 3471004H1 | 1298 | 1570 |
| 161 | 5206590H1 | 1304 | 1546 |
| 161 | 6746950H1 | 1304 | 1831 |
| 161 | 5857309H1 | 1303 | 1576 |
| 161 | 5968610H1 | 1308 | 1851 |
| 161 | g2012398 | 1308 | 1663 |
| 161 | 3469688H1 | 1317 | 1580 |
| 161 | 5857943H1 | 1354 | 1638 |
| 161 | 7070664H1 | 1508 | 1855 |
| 161 | 6416952H1 | 1519 | 1822 |
| 161 | 6894069H1 | 1526 | 2069 |
| 161 | 5013816H1 | 1533 | 1783 |
| 161 | 533363H1 | 1538 | 1775 |
| 161 | 4761822H1 | 1540 | 1812 |
| 161 | 2904083H1 | 1554 | 1850 |
| 161 | 4306790H1 | 1577 | 1687 |
| 161 | 1991775H1 | 1578 | 1797 |
| 161 | 4337965H1 | 1580 | 1827 |
| 161 | 5833237H1 | 1585 | 1750 |
| 161 | 3469095H1 | 1597 | 1855 |
| 161 | 4316943H1 | 1635 | 1803 |
| 161 | 5876761H1 | 1647 | 1931 |
| 161 | 5015603H1 | 1648 | 1813 |
| 161 | 2963466H1 | 1724 | 2028 |
| 161 | 4316745H1 | 1724 | 2020 |
| 161 | 4315891H1 | 1744 | 2022 |
| 161 | 4091142H1 | 1749 | 2023 |
| 161 | 4753020H1 | 1751 | 2007 |
| 161 | 6518022H1 | 1757 | 1838 |
| 161 | 4316639H1 | 1772 | 2046 |
| 161 | 2705655H1 | 1800 | 2083 |
| 161 | 5961924H1 | 1790 | 2298 |
| 161 | 4695643H1 | 1820 | 2036 |
| 161 | 2707689H1 | 1896 | 2192 |
| 161 | 4342587H1 | 1979 | 2088 |
| 161 | 6770516H1 | 1988 | 2569 |
| 161 | 4342620H1 | 1992 | 2246 |
| 161 | 6882093J1 | 1996 | 2568 |
| 161 | 6886296J1 | 2001 | 2481 |
| 161 | 7253724H1 | 2024 | 2531 |
| 161 | 2423349H1 | 2027 | 2272 |
| 161 | 4316734H1 | 2032 | 2342 |
| 161 | 4256579H1 | 2036 | 2265 |
| 161 | 6763709H1 | 2023 | 2568 |
| 161 | 6771870J1 | 2031 | 2578 |
| 161 | 4772777H1 | 2046 | 2149 |
| 161 | 6740527H1 | 2041 | 2522 |
| 161 | 4691683H1 | 2058 | 2293 |
| 161 | 5205527H1 | 2058 | 2293 |
| 161 | 5575884H1 | 2060 | 2251 |
| 161 | 3466389H1 | 2061 | 2306 |
| 161 | 5955966H1 | 2315 | 2492 |
| 161 | 5205250H2 | 2316 | 2479 |
| 161 | 4776444H1 | 2311 | 2566 |
| 161 | 6517485H1 | 2347 | 2825 |
| 161 | 2292420H1 | 2408 | 2647 |
| 161 | 6552809H1 | 2494 | 2887 |
| 161 | 6552988H1 | 2494 | 2887 |
| 161 | 6552963H1 | 2494 | 2887 |
| 161 | 4619412H1 | 2571 | 2791 |
| 161 | 4775811H1 | 2566 | 2801 |
| 161 | g1194667 | 2595 | 2887 |
| 161 | 5405506H1 | 2604 | 2840 |
| 161 | 4314575H1 | 2614 | 2895 |
| 161 | 1289406H1 | 2619 | 2877 |
| 161 | g1190424 | 2629 | 2887 |
| 161 | g1071637 | 2650 | 2858 |
| 161 | 4773458H1 | 2681 | 2887 |
| 161 | 4756793H1 | 2681 | 2826 |
| 161 | 4341024H1 | 2694 | 2874 |
| 161 | 4298642H1 | 2694 | 2887 |
| 161 | 1596024H1 | 2694 | 2887 |
| 161 | 552947H1 | 2699 | 2887 |
| 161 | 5371715H1 | 2713 | 2886 |
| 161 | 5447990H2 | 2737 | 2906 |
| 161 | 667160H1 | 2739 | 2887 |
| 162 | 668331H1 | 2789 | 2883 |
| 161 | 5657894H1 | 2793 | 2887 |
| 162 | 6127609T8 | 1 | 306 |
| 162 | 6959915H1 | 108 | 561 |
| 163 | 5914133H1 | 1 | 282 |
| 163 | 5914133F6 | 1 | 630 |
| 163 | 5321108F9 | 1 | 537 |
| 163 | 5914133F8 | 1 | 391 |
| 163 | 5914133T6 | 52 | 609 |
| 164 | 5911540F8 | 1 | 460 |
| 164 | 5911540H1 | 1 | 250 |
| 164 | 5911540T8 | 78 | 570 |
| 165 | 5905252F8 | 1 | 497 |
| 165 | 5905252F6 | 35 | 576 |
| 165 | 5905252H1 | 35 | 313 |
| 165 | 5905252T6 | 376 | 821 |
| 166 | 4020439F8 | 1 | 391 |
| 166 | 2773907F6 | 1 | 173 |
| 166 | 2773907H1 | 1 | 146 |
| 166 | 4020439H1 | 1 | 115 |
| 166 | 4020439T8 | 6 | 503 |
| 166 | 2773907T6 | 129 | 435 |
| 166 | g820143 | 313 | 435 |
| 167 | 6919815H1 | 1 | 89 |
| 167 | 6100456H1 | 12 | 272 |
| 167 | 5840353H1 | 19 | 294 |
| 167 | 3055393H1 | 1 | 77 |
| 167 | 4528621H1 | 3 | 250 |
| 167 | 482608H1 | 28 | 255 |
| 167 | 3477717H1 | 3 | 119 |
| 167 | 2472906H1 | 3 | 240 |
| 167 | 2444808H1 | 4 | 234 |
| 167 | 3167552H1 | 6 | 74 |
| 167 | 2615139H1 | 9 | 226 |
| 167 | 3199504H1 | 13 | 106 |
| 167 | 2455307H1 | 10 | 197 |
| 167 | 2428111H1 | 11 | 248 |
| 167 | 3458104H1 | 12 | 270 |
| 167 | 3596416H1 | 12 | 327 |
| 167 | 2453021H1 | 12 | 213 |
| 167 | 4298232H1 | 12 | 271 |
| 167 | g1956274 | 35 | 272 |
| 167 | g1880655 | 12 | 256 |
| 167 | 3458004H1 | 12 | 279 |
| 167 | 5694361H1 | 12 | 286 |
| 167 | g2026069 | 15 | 306 |
| 167 | 477069H1 | 15 | 278 |
| 167 | 1918196H1 | 15 | 293 |
| 167 | 5587272H1 | 18 | 282 |
| 167 | 4997069H1 | 18 | 284 |
| 167 | 1551008H1 | 18 | 211 |
| 167 | 5347226H1 | 19 | 275 |
| 167 | 6819761H1 | 19 | 616 |
| 167 | 5117110H1 | 20 | 289 |
| 167 | 3447366H2 | 19 | 275 |
| 167 | 2445594H1 | 19 | 249 |
| 167 | 4622780H1 | 19 | 288 |
| 167 | 2111981H1 | 20 | 282 |
| 167 | 552401H1 | 20 | 258 |
| 167 | 2461616H1 | 19 | 197 |
| 167 | 5379341H1 | 19 | 275 |
| 167 | 3820792H1 | 20 | 310 |
| 167 | 4347282H1 | 21 | 264 |
| 167 | 1322148H1 | 22 | 257 |
| 167 | 4222393H1 | 23 | 304 |
| 167 | 4223817H1 | 22 | 336 |
| 167 | 5843163H1 | 20 | 289 |
| 167 | 3657588H1 | 23 | 218 |
| 167 | 4721051H1 | 25 | 271 |
| 167 | 4790883H1 | 21 | 278 |
| 167 | 5377733H1 | 24 | 280 |
| 167 | g1638522 | 24 | 353 |
| 167 | 3596245H1 | 25 | 318 |
| 167 | 781633H1 | 25 | 270 |
| 167 | 5374490H1 | 26 | 286 |
| 167 | 2161371H1 | 31 | 277 |
| 167 | 3597248H1 | 32 | 267 |
| 167 | 2934534H1 | 36 | 172 |
| 167 | 4386768H1 | 37 | 326 |
| 167 | 3399780H1 | 126 | 191 |
| 167 | 6399947H1 | 126 | 272 |
| 167 | 6897251H1 | 166 | 634 |
| 167 | 4431669H2 | 173 | 312 |
| 167 | g1779781 | 330 | 692 |
| 167 | g826488 | 330 | 717 |
| 167 | 4794163H1 | 346 | 595 |
| 167 | 4880181H1 | 349 | 609 |
| 167 | g889083 | 366 | 638 |
| 167 | 6819761J1 | 396 | 1013 |
| 167 | g916666 | 408 | 513 |
| 167 | 4249123H1 | 452 | 539 |
| 167 | 6481276H1 | 584 | 800 |
| 167 | 4191747H1 | 624 | 709 |
| 167 | 638004H1 | 648 | 753 |
| 167 | 1452178H1 | 676 | 753 |
| 167 | g2615681 | 692 | 753 |
| 167 | g1665347 | 697 | 753 |
| 167 | g2558364 | 697 | 753 |
| 168 | 6795278H1 | 184 | 698 |
| 168 | 6796542H1 | 1 | 546 |
| 168 | 6796380H1 | 8 | 563 |
| 168 | 6795463H1 | 8 | 523 |
| 168 | 3941984H1 | 26 | 315 |
| 168 | g1260435 | 112 | 287 |
| 168 | 6798249H1 | 184 | 635 |
| 168 | 6791366H1 | 238 | 804 |
| 168 | 6790685H1 | 239 | 699 |
| 168 | 1242854H1 | 698 | 885 |
| 169 | 2904954T6 | 1 | 522 |
| 169 | 4739603H1 | 11 | 303 |
| 169 | 5614905H1 | 213 | 492 |
| 169 | g2006850 | 277 | 557 |
| 169 | 2008385H1 | 464 | 562 |
| 169 | 2014576T6 | 464 | 523 |
| 169 | 2014576H1 | 464 | 586 |
| 169 | 3294227T6 | 223 | 557 |
| 169 | 2014576R6 | 464 | 553 |
| 169 | 1716729H1 | 501 | 556 |
| 170 | 3966795F6 | 1 | 365 |
| 170 | 3966795H1 | 1 | 267 |
| 170 | 3274864H1 | 14 | 268 |
| 170 | 3966795T6 | 23 | 659 |
| 171 | 3033193F6 | 1 | 272 |
| 171 | 3033193H1 | 1 | 216 |
| 171 | 3033193T6 | 129 | 445 |
| 172 | 5908301F8 | 1 | 519 |
| 172 | 5908301H1 | 1 | 311 |
| 172 | 6271267H2 | 24 | 492 |
| 172 | 5908301T9 | 248 | 586 |
| 173 | 5907939T9 | 1 | 517 |
| 173 | 5907939F8 | 1 | 551 |
| 173 | 5907939H1 | 1 | 310 |
| 173 | 6271008H2 | 10 | 483 |
| 174 | 5912415F8 | 1 | 376 |
| 174 | 5912415H1 | 1 | 299 |
| 174 | 5912415F6 | 12 | 565 |
| 174 | 5912415T9 | 66 | 535 |
| 175 | 4119207F6 | 1 | 336 |
| 175 | 4119207T6 | 1 | 336 |
| 175 | 4119207H1 | 1 | 175 |
| 176 | 5905477F6 | 1 | 564 |
| 176 | 5905477H1 | 1 | 271 |
| 176 | 5905477T9 | 411 | 932 |
| 177 | 5907791F8 | 1 | 360 |
| 177 | 5907791H1 | 1 | 280 |
| 177 | 5907791F6 | 1 | 305 |
| 177 | 5907791T9 | 155 | 682 |
| 177 | 5907791T6 | 248 | 733 |
| 178 | 4770137H1 | 1 | 144 |
| 178 | 5564253H1 | 2 | 235 |
| 178 | 606101H1 | 1 | 169 |
| 178 | 4650476H1 | 1 | 271 |
| 178 | 592893H1 | 2 | 130 |
| 178 | 781453H1 | 17 | 276 |
| 178 | 2793564H1 | 28 | 317 |
| 178 | 888943H1 | 151 | 287 |
| 178 | 6589872H1 | 194 | 698 |
| 178 | 2190828T6 | 207 | 699 |
| 178 | 1503654H1 | 225 | 491 |
| 178 | 4200384H1 | 388 | 662 |
| 179 | 5911540F8 | 1 | 460 |
| 179 | 5911540H1 | 1 | 250 |
| 179 | 5911540T9 | 27 | 568 |
| 179 | 5911540T8 | 78 | 569 |
| 180 | g4325750 | 1 | 103 |
| 180 | 4290049F6 | 1 | 353 |
| 180 | 4290049H1 | 1 | 124 |
| 180 | 5493752H1 | 172 | 444 |
| 181 | 6729842H1 | 1 | 412 |
| 181 | 5401350H1 | 1 | 105 |
| 181 | 6057617H1 | 56 | 643 |
| 181 | 5401350T9 | 82 | 666 |
| 181 | g3214092 | 406 | 782 |
| 181 | 3524102H1 | 479 | 779 |
| 182 | 7030475H1 | 1 | 524 |
| 182 | 7030327H1 | 3 | 376 |
| 183 | 5271230H1 | 1485 | 1748 |
| 183 | 240641H1 | 1485 | 1657 |
| 183 | 2948213H1 | 1486 | 1768 |
| 183 | g1291432 | 1492 | 1896 |
| 183 | 353018H1 | 1496 | 1708 |
| 183 | 349497H1 | 1496 | 1680 |
| 183 | g2276981 | 1533 | 1859 |
| 183 | 6804414J1 | 1537 | 2080 |
| 183 | 3499213H1 | 1537 | 1841 |
| 183 | g2537462 | 1542 | 1981 |
| 183 | 1551014H1 | 1549 | 1763 |
| 183 | 808076H1 | 1554 | 1769 |
| 183 | 2307944H1 | 1557 | 1762 |
| 183 | g2222952 | 1559 | 1851 |
| 183 | 2804178H1 | 1565 | 1852 |
| 183 | 6801505J1 | 1566 | 1881 |
| 183 | g888315 | 1569 | 1927 |
| 183 | g2056786 | 1570 | 2035 |
| 183 | 6901949H1 | 1 | 518 |
| 183 | 4183549H1 | 91 | 259 |
| 183 | 6980581H1 | 104 | 452 |
| 183 | 5839778H1 | 107 | 359 |
| 183 | 808102H1 | 172 | 406 |
| 183 | 4323252H1 | 224 | 469 |
| 183 | g2003585 | 258 | 517 |
| 183 | g900863 | 361 | 447 |
| 183 | 6979927H1 | 437 | 785 |
| 183 | 582110H1 | 487 | 739 |
| 183 | 3323870H1 | 499 | 778 |
| 183 | 2115479R6 | 515 | 913 |
| 183 | 4779981H1 | 538 | 793 |
| 183 | 1818066H1 | 552 | 808 |
| 183 | 1818066F6 | 552 | 893 |
| 183 | g4438745 | 584 | 797 |
| 183 | 5450643H1 | 614 | 851 |
| 183 | 7066292H1 | 636 | 1078 |
| 183 | 3538459H1 | 648 | 855 |
| 183 | 2115479H1 | 655 | 913 |
| 183 | 4090518H1 | 669 | 864 |
| 183 | 6077621H1 | 686 | 797 |
| 183 | 4947628H1 | 702 | 832 |
| 183 | 4759655H1 | 713 | 993 |
| 183 | 1699317H1 | 715 | 931 |
| 183 | 4303659H1 | 740 | 991 |
| 183 | 591324R6 | 752 | 1180 |
| 183 | 591324H1 | 752 | 975 |
| 183 | 591174H1 | 752 | 955 |
| 183 | 5998787H1 | 772 | 1179 |
| 183 | 1672160H1 | 778 | 991 |
| 183 | 1672152H1 | 778 | 991 |
| 183 | 4694794H1 | 784 | 1045 |
| 183 | 2972664H2 | 782 | 1078 |
| 183 | 1718469H1 | 795 | 1006 |
| 183 | 1718480H1 | 795 | 998 |
| 183 | 3270027H1 | 802 | 1059 |
| 183 | 927251R1 | 806 | 1419 |
| 183 | 927251H1 | 806 | 1058 |
| 183 | g1986696 | 809 | 992 |
| 183 | 5102583H1 | 821 | 1068 |
| 183 | 7094153H1 | 822 | 1103 |
| 183 | 5948962H1 | 843 | 1137 |
| 183 | 1571253H1 | 845 | 1013 |
| 183 | 5371617H1 | 860 | 1101 |
| 183 | 3784982H1 | 895 | 1195 |
| 183 | 6980693H1 | 897 | 1257 |
| 183 | 4770011H1 | 901 | 1166 |
| 183 | 1695218H1 | 903 | 1119 |
| 183 | 3697260H1 | 962 | 1179 |
| 183 | 1331860H1 | 981 | 1179 |
| 183 | g1644929 | 1018 | 1179 |
| 183 | g1880762 | 1046 | 1163 |
| 183 | 6804414H1 | 1065 | 1601 |
| 183 | 1722938H1 | 1074 | 1327 |
| 183 | 5451478H1 | 1104 | 1348 |
| 183 | 1500938H1 | 1108 | 1304 |
| 183 | 5950551H1 | 1120 | 1413 |
| 183 | 3376840H1 | 1121 | 1367 |
| 183 | 5950519H1 | 1121 | 1320 |
| 183 | 4193013H1 | 1121 | 1438 |
| 183 | 4556920H1 | 1124 | 1261 |
| 183 | g1046489 | 1143 | 1470 |
| 183 | g1046497 | 1143 | 1455 |
| 183 | 1283213H1 | 1202 | 1390 |
| 183 | 1283213F6 | 1202 | 1658 |
| 183 | 4225341H1 | 1206 | 1487 |
| 183 | 6714888H1 | 1214 | 1778 |
| 183 | 1346996H1 | 1259 | 1485 |
| 183 | 1348304H1 | 1259 | 1481 |
| 183 | 1348303H1 | 1259 | 1481 |
| 183 | 6954001H1 | 1261 | 1764 |
| 183 | g2107139 | 1290 | 1761 |
| 183 | 4720048H1 | 1308 | 1583 |
| 183 | 1720725H1 | 1328 | 1538 |
| 183 | 1722139H1 | 1328 | 1553 |
| 183 | 5970873H1 | 1334 | 1887 |
| 183 | 4693221H1 | 1344 | 1601 |
| 183 | 2571078H1 | 1343 | 1591 |
| 183 | 6121921H1 | 1351 | 1904 |
| 183 | 4215895H1 | 1363 | 1625 |
| 183 | g2188904 | 1372 | 1857 |
| 183 | g1646485 | 1374 | 1468 |
| 183 | 004844H1 | 1386 | 1669 |
| 183 | 6313521H1 | 1386 | 1950 |
| 183 | 2427675H1 | 1392 | 1622 |
| 183 | 5478565H1 | 1401 | 1608 |
| 183 | 5480821H1 | 1402 | 1635 |
| 183 | 5483319H1 | 1402 | 1501 |
| 183 | 5322481H1 | 1403 | 1641 |
| 183 | 5948083H1 | 1409 | 1714 |
| 183 | 1996549H1 | 1415 | 1554 |
| 183 | 1996549R6 | 1415 | 1751 |
| 183 | 5405753H1 | 1434 | 1586 |
| 183 | 5293687H2 | 1434 | 1680 |
| 183 | 935623R1 | 1465 | 1989 |
| 183 | 936611H1 | 1465 | 1767 |
| 183 | 935623H1 | 1465 | 1708 |
| 183 | 3409340H1 | 1473 | 1629 |
| 183 | 1819126H1 | 1480 | 1748 |
| 183 | 1819126F6 | 1480 | 1979 |
| 183 | g2053565 | 1480 | 1888 |
| 183 | g2209615 | 1481 | 1929 |
| 183 | 5879274H1 | 1482 | 1754 |
| 183 | 4570795H1 | 2032 | 2292 |
| 183 | g1046490 | 2044 | 2349 |
| 183 | 1416514H1 | 2046 | 2291 |
| 183 | 4193606H1 | 2048 | 2324 |
| 183 | g1858344 | 2051 | 2338 |
| 183 | 5021880T1 | 2055 | 2300 |
| 183 | 1281333H1 | 2058 | 2294 |
| 183 | 2222539H1 | 2060 | 2298 |
| 183 | 2222539F6 | 2060 | 2338 |
| 183 | 2222539T6 | 2061 | 2300 |
| 183 | 5341229H1 | 2066 | 2265 |
| 183 | 2659133H1 | 2067 | 2280 |
| 183 | g3871000 | 2069 | 2338 |
| 183 | g4524292 | 2077 | 2340 |
| 183 | g1858279 | 2081 | 2342 |
| 183 | 1301182H1 | 2082 | 2343 |
| 183 | 2256984H1 | 2105 | 2338 |
| 183 | 6498361H1 | 2106 | 2338 |
| 183 | 2574115H1 | 2106 | 2337 |
| 183 | 2273027H1 | 2107 | 2310 |
| 183 | g4899838 | 2119 | 2346 |
| 183 | g3051313 | 2128 | 2338 |
| 183 | 3142955H1 | 2158 | 2340 |
| 183 | 2878176H1 | 2164 | 2339 |
| 183 | 3271618H1 | 2181 | 2337 |
| 183 | g2958193 | 2190 | 2340 |
| 183 | 2247168H1 | 2197 | 2344 |
| 183 | 2246357H1 | 2225 | 2339 |
| 183 | 2247256H1 | 2254 | 2339 |
| 183 | 3812939H1 | 2274 | 2338 |
| 183 | 5103254H1 | 2283 | 2339 |
| 183 | 7064872H1 | 1575 | 2105 |
| 183 | 6818666H1 | 1579 | 1827 |
| 183 | 4726069H1 | 1581 | 1854 |
| 183 | g4662834 | 1586 | 2036 |
| 183 | g1182379 | 1589 | 1804 |
| 183 | g4874675 | 1588 | 2033 |
| 183 | 5661656H1 | 1589 | 1849 |
| 183 | 6868292H1 | 1591 | 2060 |
| 183 | 3706916H1 | 1605 | 1904 |
| 183 | g2208265 | 1633 | 2033 |
| 183 | 2424009H1 | 1649 | 1907 |
| 183 | 1470490H1 | 1663 | 1849 |
| 183 | 797359H1 | 1664 | 1903 |
| 183 | 3245252H1 | 1669 | 1936 |
| 183 | 4781187H1 | 1671 | 1939 |
| 183 | g1688385 | 1690 | 2024 |
| 183 | 1413737H1 | 1712 | 1978 |
| 183 | g2279273 | 1737 | 2033 |
| 183 | g1146486 | 1741 | 1926 |
| 183 | g1644873 | 1745 | 2038 |
| 183 | g888316 | 1749 | 2033 |
| 183 | 099594H1 | 1754 | 1972 |
| 183 | 3996904H1 | 1754 | 1913 |
| 183 | 3916961H1 | 1759 | 2048 |
| 183 | 3917411H1 | 1759 | 2036 |
| 183 | 6801505H1 | 1759 | 2125 |
| 183 | 910557H1 | 1785 | 1850 |
| 183 | 2921975H1 | 1788 | 2061 |
| 183 | 5003193H1 | 1790 | 2060 |
| 183 | g1442965 | 1791 | 2033 |
| 183 | 5018209H1 | 1797 | 1957 |
| 183 | 3246162H1 | 1797 | 2036 |
| 183 | 1344364H1 | 1798 | 2013 |
| 183 | 1344388H1 | 1798 | 2036 |
| 183 | 5928305H1 | 1801 | 2111 |
| 183 | 683816H1 | 1805 | 2040 |
| 183 | 5451469H1 | 1808 | 2048 |
| 183 | 2402750H1 | 1811 | 1919 |
| 183 | 1851976H1 | 1816 | 2034 |
| 183 | 3702341H1 | 1825 | 2066 |
| 183 | 2963989H1 | 1819 | 2096 |
| 183 | g1441785 | 1826 | 2091 |
| 183 | 5777701H1 | 1828 | 2105 |
| 183 | g3898324 | 1831 | 2042 |
| 183 | 1819126T6 | 1843 | 2298 |
| 183 | 1484988H1 | 1847 | 2095 |
| 183 | 2106229T6 | 1851 | 2298 |
| 183 | g4762523 | 1859 | 2033 |
| 183 | g4620507 | 1860 | 2039 |
| 183 | 1964264H1 | 1872 | 2033 |
| 183 | 1964264R6 | 1872 | 2040 |
| 183 | 1964264T6 | 1872 | 2002 |
| 183 | g2945963 | 1876 | 2340 |
| 183 | 4696529H1 | 1885 | 2095 |
| 183 | g4564438 | 1886 | 2339 |
| 183 | g3741810 | 1886 | 2341 |
| 183 | 2913361H1 | 1889 | 1977 |
| 183 | 6862628H1 | 1894 | 2033 |
| 183 | g3742173 | 1905 | 2340 |
| 183 | g4083420 | 1910 | 2338 |
| 183 | g3678703 | 1912 | 2342 |
| 183 | 6327536H1 | 1912 | 2340 |
| 183 | g2910125 | 1916 | 2341 |
| 183 | g3308355 | 1919 | 2342 |
| 183 | 2556534H1 | 1933 | 2178 |
| 183 | g3922126 | 1935 | 2338 |
| 183 | g4083267 | 1936 | 2338 |
| 183 | g4083582 | 1951 | 2338 |
| 183 | g1646486 | 1958 | 2340 |
| 183 | g2902884 | 1998 | 2339 |
| 183 | 953800T1 | 2000 | 2301 |
| 183 | g895530 | 2006 | 2338 |
| 183 | 953800H1 | 2012 | 2296 |
| 183 | 953800R1 | 2012 | 2338 |
| 183 | 4977128H1 | 2022 | 2281 |
| 183 | g1046498 | 2026 | 2338 |
| 184 | 3497339T6 | 1 | 492 |
| 184 | g1060678 | 211 | 436 |
| 184 | 6258709H1 | 213 | 444 |
| 184 | 2570554H1 | 239 | 484 |
| 184 | 2570554R6 | 239 | 634 |
| 184 | 6200064H1 | 459 | 921 |
| 184 | g734035 | 473 | 792 |
| 184 | 1400373F6 | 474 | 1007 |
| 184 | 1400373H1 | 474 | 733 |
| 184 | g2167556 | 488 | 897 |
| 184 | 6389638H1 | 542 | 823 |
| 184 | 6344656H1 | 598 | 885 |
| 184 | 6344688H1 | 606 | 888 |
| 184 | 6328470H1 | 664 | 1018 |
| 184 | g4148622 | 723 | 1072 |
| 184 | 6338470H1 | 793 | 1018 |
| 184 | 3784456H1 | 876 | 1200 |
| 184 | 3781165H1 | 943 | 1049 |
| 184 | g733951 | 1031 | 1403 |
| 184 | g734304 | 1149 | 1333 |
| 184 | 3554877H1 | 1 | 294 |
| 185 | 4111213H1 | 1 | 243 |
| 185 | 5624259R8 | 1 | 350 |
| 185 | 3139315H1 | 126 | 427 |
| 185 | 5545642H1 | 130 | 363 |
| 185 | 3141553H1 | 139 | 412 |
| 185 | g564381 | 139 | 369 |
| 185 | 987690H1 | 154 | 424 |
| 185 | 4833664H1 | 169 | 319 |
| 185 | 116132H1 | 214 | 403 |
| 185 | 3691576H1 | 244 | 399 |
| 185 | 1667502F6 | 265 | 644 |
| 185 | 3283023H1 | 267 | 528 |
| 185 | 3959293H2 | 290 | 564 |
| 185 | 2675859H1 | 347 | 596 |
| 185 | 2676108H1 | 347 | 594 |
| 185 | 2676122H1 | 347 | 591 |
| 185 | 5301231H1 | 387 | 569 |
| 185 | 1598950H1 | 387 | 567 |
| 185 | 2352332H1 | 456 | 626 |
| 185 | 5038795H1 | 459 | 689 |
| 185 | 2534205H1 | 459 | 705 |
| 185 | 3252993H1 | 464 | 711 |
| 185 | 1742040H1 | 490 | 721 |
| 185 | 4592168H1 | 498 | 648 |
| 185 | g29244 | 1 | 255 |
| 185 | 2635196H1 | 1 | 249 |
| 186 | 6428735H1 | 867 | 1184 |
| 187 | 2525961F6 | 1 | 449 |
| 187 | 2525961H1 | 2 | 243 |
| 187 | 6560586H1 | 64 | 613 |
| 187 | g1357976 | 235 | 523 |
| 187 | 2525961T6 | 253 | 677 |
| 187 | g3245391 | 532 | 894 |
| 188 | g1277774 | 1 | 322 |
| 188 | g2186366 | 1 | 442 |
| 188 | 5533987H1 | 1 | 268 |
| 188 | 5320656H1 | 23 | 287 |
| 188 | 985175H1 | 22 | 276 |
| 188 | 5478163H1 | 23 | 303 |
| 188 | 5476045H1 | 23 | 301 |
| 188 | 5325512H1 | 23 | 291 |
| 188 | 5479637H1 | 23 | 159 |
| 188 | 7289635H1 | 196 | 762 |
| 188 | 6559813H1 | 603 | 1192 |
| 188 | 6858544H1 | 732 | 1210 |
| 188 | 5929387H1 | 845 | 1124 |
| 188 | 5778630H1 | 845 | 1114 |
| 188 | 4017917H1 | 851 | 1115 |
| 188 | 5206602H1 | 905 | 1185 |
| 188 | 4776841H1 | 1078 | 1295 |
| 188 | 6314480H1 | 1093 | 1213 |
| 189 | 70395867D1 | 1 | 542 |
| 189 | 2525961F6 | 4 | 452 |
| 189 | 2525961H1 | 5 | 246 |
| 189 | 70395557D1 | 40 | 642 |
| 189 | 70397658D1 | 59 | 589 |
| 189 | 6560586H1 | 67 | 616 |
| 189 | 70396453D1 | 72 | 478 |
| 189 | 70396167D1 | 119 | 654 |
| 189 | 70396459D1 | 118 | 585 |
| 189 | 70396121D1 | 192 | 481 |
| 189 | g1357976 | 238 | 526 |
| 189 | 2525961T6 | 256 | 680 |
| 189 | 70396053D1 | 260 | 679 |
| 189 | 70396654D1 | 278 | 768 |
| 189 | 70395799D1 | 279 | 680 |
| 189 | 70395826D1 | 279 | 680 |
| 189 | 70397446D1 | 279 | 676 |
| 189 | 70397464D1 | 292 | 676 |
| 189 | 70394880D1 | 293 | 746 |
| 189 | 70397538D1 | 293 | 768 |
| 189 | 70396886D1 | 292 | 564 |
| 189 | 70395257D1 | 293 | 751 |
| 189 | 70395475D1 | 298 | 676 |
| 189 | 70395110D1 | 307 | 676 |
| 189 | 70395982D1 | 312 | 680 |
| 189 | 70397165D1 | 352 | 676 |
| 189 | 70396476D1 | 483 | 676 |
| 189 | g3245391 | 535 | 897 |
| 190 | 2402420H1 | 4083 | 4349 |
| 190 | 1340154H1 | 4091 | 4319 |
| 190 | 2791792R6 | 4108 | 4475 |
| 190 | 2079255T6 | 4053 | 4414 |
| 190 | g2884989 | 4111 | 4451 |
| 190 | g1189027 | 4060 | 4458 |
| 190 | 2412005H1 | 4120 | 4352 |
| 190 | g2354871 | 4123 | 4450 |
| 190 | g2350487 | 4133 | 4457 |
| 190 | g5662773 | 4156 | 4466 |
| 190 | g5639034 | 4061 | 4453 |
| 190 | g816766 | 4165 | 4460 |
| 190 | 3041690H1 | 4157 | 4451 |
| 190 | 977648R1 | 4063 | 4431 |
| 190 | g1193159 | 4182 | 4458 |
| 190 | 977648T1 | 4063 | 4415 |
| 190 | 977648H1 | 4063 | 4386 |
| 190 | 978306H1 | 4063 | 4319 |
| 190 | g2051455 | 4184 | 4460 |
| 190 | 5280171H1 | 4187 | 4442 |
| 190 | g3886427 | 4204 | 4347 |
| 190 | 1381539H1 | 4210 | 4457 |
| 190 | 552969H1 | 4210 | 4458 |
| 190 | 2760603H1 | 4210 | 4453 |
| 190 | g3647431 | 4212 | 4454 |
| 190 | 1917113H1 | 4245 | 4457 |
| 190 | 4787118H1 | 4063 | 4343 |
| 190 | g892648 | 4259 | 4458 |
| 190 | g2229339 | 4272 | 4456 |
| 190 | 1942308H1 | 4292 | 4461 |
| 190 | 1942315H1 | 4292 | 4461 |
| 190 | 1941519H1 | 4327 | 4457 |
| 190 | 5007945H1 | 4389 | 4457 |
| 190 | 1979718T6 | 4069 | 4419 |
| 190 | 240941H1 | 4066 | 4182 |
| 190 | 1979718R6 | 4069 | 4452 |
| 190 | 1979718H1 | 4069 | 4174 |
| 190 | g682911 | 4083 | 4457 |
| 190 | 4465894H1 | 3788 | 4048 |
| 190 | 2819748T6 | 3815 | 4417 |
| 190 | 3445036H1 | 3816 | 4085 |
| 190 | 6735779H1 | 3822 | 4433 |
| 190 | 2279717T6 | 3824 | 4418 |
| 190 | 3720714H1 | 3832 | 4020 |
| 190 | 4828820H1 | 3844 | 4119 |
| 190 | 1369089H1 | 3847 | 4077 |
| 190 | 1833781H1 | 3850 | 4130 |
| 190 | 1965941T6 | 3865 | 4422 |
| 190 | 2559627H1 | 3868 | 3984 |
| 190 | 2285422H1 | 3881 | 4083 |
| 190 | 5483595H1 | 3904 | 4084 |
| 190 | 5322590H1 | 3904 | 4157 |
| 190 | g892647 | 3925 | 4226 |
| 190 | 4030718T6 | 3927 | 4433 |
| 190 | 6476518H1 | 3939 | 4460 |
| 190 | 5013604H1 | 3940 | 4221 |
| 190 | 2245864H1 | 3949 | 4223 |
| 190 | 2301055T6 | 3955 | 4416 |
| 190 | 2292517T6 | 3967 | 4419 |
| 190 | g5444044 | 3982 | 4452 |
| 190 | g5436138 | 3984 | 4465 |
| 190 | g3427279 | 3990 | 4459 |
| 190 | 6612393H1 | 4008 | 4444 |
| 190 | g6044228 | 4017 | 4457 |
| 190 | g4650722 | 4020 | 4460 |
| 190 | g5109970 | 4020 | 4459 |
| 190 | g2656315 | 4021 | 4456 |
| 190 | g3277136 | 4026 | 4453 |
| 190 | 615565H1 | 4040 | 4314 |
| 190 | g1192291 | 4042 | 4126 |
| 190 | g4002109 | 4047 | 4457 |
| 190 | 933913R1 | 3411 | 3813 |
| 190 | 933913H1 | 3411 | 3683 |
| 190 | 3056157H1 | 3442 | 3663 |
| 190 | 6853558H1 | 3446 | 3911 |
| 190 | 1267149F1 | 3446 | 3913 |
| 190 | 1267149H1 | 3446 | 3659 |
| 190 | 1413302H1 | 3453 | 3663 |
| 190 | 4092170H1 | 3473 | 3749 |
| 190 | 1961533H1 | 3491 | 3760 |
| 190 | 1961533R6 | 3491 | 3663 |
| 190 | 464481H1 | 3491 | 3663 |
| 190 | g2141846 | 3498 | 3817 |
| 190 | 1373705H1 | 3514 | 3748 |
| 190 | 3961925H1 | 3584 | 3641 |
| 190 | 5285788H1 | 3610 | 3739 |
| 190 | 2301055R6 | 3663 | 3918 |
| 190 | 1965941R6 | 3692 | 4177 |
| 190 | 3991277H1 | 3692 | 3981 |
| 190 | 1923669H1 | 3692 | 3923 |
| 190 | 2301055H1 | 3692 | 3919 |
| 190 | 1965941H1 | 3692 | 3916 |
| 190 | 1833543H1 | 3692 | 3890 |
| 190 | 927374H1 | 3696 | 3966 |
| 190 | 4511590H1 | 3696 | 3931 |
| 190 | 6517421H1 | 3703 | 4233 |
| 190 | 1551235T6 | 3716 | 4417 |
| 190 | 2674692H1 | 3731 | 3965 |
| 190 | 2288745H1 | 3734 | 3969 |
| 190 | 4311971H1 | 3740 | 4037 |
| 190 | 6118751H1 | 3753 | 4264 |
| 190 | 6119992H1 | 3753 | 4329 |
| 190 | 6126908H1 | 3753 | 4255 |
| 190 | g4629955 | 3755 | 3976 |
| 190 | 2157206H1 | 3772 | 4029 |
| 190 | 71226132V1 | 2110 | 2748 |
| 190 | g3092783 | 2129 | 2371 |
| 190 | 70863741V1 | 2130 | 2716 |
| 190 | 2079255F6 | 2130 | 2513 |
| 190 | 2079255H1 | 2130 | 2367 |
| 190 | 6167128H1 | 2173 | 2701 |
| 190 | 70856205V1 | 2176 | 2782 |
| 190 | 6482958H1 | 2180 | 2277 |
| 190 | 70149143V1 | 2191 | 2726 |
| 190 | 5372491H1 | 2218 | 2355 |
| 190 | 71226268V1 | 2225 | 2707 |
| 190 | g5234288 | 2245 | 2718 |
| 190 | g389953 | 2249 | 2637 |
| 190 | g769298 | 2249 | 2567 |
| 190 | 579131H1 | 2257 | 2504 |
| 190 | 579198H1 | 2257 | 2503 |
| 190 | g3308511 | 2277 | 2721 |
| 190 | 083837H1 | 2276 | 2489 |
| 190 | g4534587 | 2287 | 2674 |
| 190 | g4188225 | 2296 | 2712 |
| 190 | 3380281H1 | 2315 | 2563 |
| 190 | g3870352 | 2316 | 2713 |
| 190 | 3086136H1 | 2326 | 2531 |
| 190 | g3228885 | 2328 | 2714 |
| 190 | g2541163 | 2344 | 2721 |
| 190 | g3884137 | 2348 | 2721 |
| 190 | 1819282F6 | 2363 | 2713 |
| 190 | 1819282H1 | 2363 | 2618 |
| 190 | 6492522H1 | 2369 | 2978 |
| 190 | g875856 | 2373 | 2721 |
| 190 | 4176829H1 | 2371 | 2638 |
| 190 | 4176860H1 | 2371 | 2634 |
| 190 | 70864219V1 | 2391 | 2984 |
| 190 | 1819282T6 | 2405 | 2666 |
| 190 | g819854 | 2421 | 2722 |
| 190 | g561301 | 2418 | 2717 |
| 190 | 70145373V1 | 2426 | 2719 |
| 190 | 4670647H1 | 2442 | 2720 |
| 190 | 70855556V1 | 2465 | 3045 |
| 190 | 6844792H1 | 2498 | 2713 |
| 190 | 3724780H1 | 2530 | 2833 |
| 190 | 4822074H1 | 2542 | 2658 |
| 190 | g794631 | 2547 | 2721 |
| 190 | g708639 | 2550 | 2858 |
| 190 | 70146025V1 | 2559 | 3033 |
| 190 | g691143 | 2573 | 2932 |
| 190 | g3096354 | 2577 | 2716 |
| 190 | 70855455V1 | 2586 | 3160 |
| 190 | 4259987H1 | 2619 | 2702 |
| 190 | 6545104H1 | 2633 | 2713 |
| 190 | g874560 | 2650 | 2722 |
| 190 | 4612918H1 | 2655 | 2837 |
| 190 | 4144852H1 | 2661 | 2942 |
| 190 | 985756R1 | 2688 | 3124 |
| 190 | 985756H1 | 2688 | 2979 |
| 190 | 5570469H1 | 2726 | 2925 |
| 190 | 6323423H1 | 2731 | 2988 |
| 190 | 985756R6 | 2731 | 2808 |
| 190 | 6835880H1 | 2740 | 3320 |
| 190 | 6742687H1 | 2750 | 3268 |
| 190 | 5350646H1 | 2785 | 3035 |
| 190 | 71227966V1 | 2797 | 3000 |
| 190 | 3091359H1 | 2803 | 3085 |
| 190 | 985756T6 | 2829 | 3266 |
| 190 | g1198696 | 2825 | 3127 |
| 190 | g3416884 | 2871 | 3306 |
| 190 | 3695945H1 | 2881 | 3163 |
| 190 | 5377540H1 | 2893 | 3153 |
| 190 | g5363556 | 2895 | 3301 |
| 190 | g618368 | 2909 | 3252 |
| 190 | 1305496T6 | 2927 | 3263 |
| 190 | 1551235R6 | 2945 | 3460 |
| 190 | g530325 | 2945 | 3279 |
| 190 | 1551235H1 | 2945 | 3153 |
| 190 | 4311884H1 | 2976 | 3299 |
| 190 | 3659340H1 | 2985 | 3090 |
| 190 | 6125263H1 | 2989 | 3523 |
| 190 | 1984710H1 | 2986 | 3241 |
| 190 | 6124418H1 | 2989 | 3449 |
| 190 | 1008121H1 | 2998 | 3264 |
| 190 | 4402539H1 | 3002 | 3244 |
| 190 | 3518554H1 | 3018 | 3268 |
| 190 | 3345944H1 | 3032 | 3288 |
| 190 | 2819748F6 | 3075 | 3394 |
| 190 | 2819748H1 | 3077 | 3231 |
| 190 | 5660765H1 | 3096 | 3330 |
| 190 | 71225736V1 | 3132 | 3768 |
| 190 | 70857262V1 | 3133 | 3301 |
| 190 | 3785435H1 | 3154 | 3448 |
| 190 | 2279717H1 | 3183 | 3424 |
| 190 | 2279717R6 | 3183 | 3373 |
| 190 | 3469571H1 | 3207 | 3464 |
| 190 | 3356238H1 | 3209 | 3470 |
| 190 | 1805536H1 | 3212 | 3479 |
| 190 | 4160048H1 | 3214 | 3272 |
| 190 | 4942785H1 | 3234 | 3464 |
| 190 | 5585444H1 | 3235 | 3448 |
| 190 | 6269078H1 | 3252 | 3752 |
| 190 | 127072H1 | 3265 | 3456 |
| 190 | 7290084H1 | 3269 | 3683 |
| 190 | 7011256H1 | 3273 | 3657 |
| 190 | g1893973 | 3278 | 3669 |
| 190 | 3992283H1 | 3291 | 3405 |
| 190 | 3793161H1 | 3292 | 3493 |
| 190 | 3325588H1 | 3301 | 3515 |
| 190 | 1008331H1 | 3310 | 3628 |
| 190 | 2637310F6 | 3313 | 3559 |
| 190 | 2637310H1 | 3313 | 3583 |
| 190 | 4623479H1 | 3314 | 3577 |
| 190 | 3769624H1 | 3325 | 3624 |
| 190 | 3725948H1 | 3328 | 3615 |
| 190 | 3433892H1 | 3345 | 3591 |
| 190 | 3919666H1 | 3349 | 3447 |
| 190 | 5732787H1 | 3359 | 3579 |
| 190 | 3741626H1 | 3360 | 3641 |
| 190 | 5874821H1 | 3380 | 3628 |
| 190 | 2292517H1 | 3382 | 3639 |
| 190 | 3559065H1 | 3393 | 3574 |
| 190 | 3778532H1 | 3404 | 3653 |
| 190 | 6520495H1 | 3409 | 3864 |
| 190 | 5510632F6 | 1 | 402 |
| 190 | 5510632H1 | 1 | 201 |
| 190 | 6780915H1 | 41 | 512 |
| 190 | 4782751H1 | 127 | 371 |
| 190 | 4782751F6 | 127 | 187 |
| 190 | 2987413H1 | 192 | 477 |
| 190 | 6064208H1 | 203 | 464 |
| 190 | 5336504H1 | 219 | 435 |
| 190 | 5337416H1 | 219 | 473 |
| 190 | g1984325 | 271 | 504 |
| 190 | 7245264H1 | 286 | 854 |
| 190 | g3239921 | 293 | 648 |
| 190 | g2026718 | 299 | 586 |
| 190 | 7245164H1 | 302 | 823 |
| 190 | g2816869 | 320 | 565 |
| 190 | 5510632R6 | 364 | 855 |
| 190 | 4760384H1 | 491 | 575 |
| 190 | 4760384F6 | 491 | 961 |
| 190 | 1305496F6 | 798 | 1267 |
| 190 | 70145686V1 | 798 | 1046 |
| 190 | 1305496H1 | 798 | 941 |
| 190 | 3617505H1 | 819 | 1021 |
| 190 | 70861843V1 | 870 | 1297 |
| 190 | g669800 | 873 | 1196 |
| 190 | g875546 | 874 | 1129 |
| 190 | g874649 | 874 | 1176 |
| 190 | 70862573V1 | 891 | 1352 |
| 190 | 70856733V1 | 964 | 1627 |
| 190 | g771213 | 1008 | 1060 |
| 190 | 7048824H1 | 1036 | 1629 |
| 190 | 71225244V1 | 1056 | 1612 |
| 190 | 3211592H1 | 1054 | 1289 |
| 190 | 093259H1 | 1079 | 1319 |
| 190 | 926880H1 | 1120 | 1361 |
| 190 | 6163034H1 | 1127 | 1456 |
| 190 | 70860860V1 | 1134 | 1758 |
| 190 | 1366443R6 | 1134 | 1468 |
| 190 | 1366443H1 | 1134 | 1383 |
| 190 | 70145966V1 | 1141 | 1461 |
| 190 | 6448764H1 | 1151 | 1743 |
| 190 | 4960778H1 | 1157 | 1430 |
| 190 | 71226715V1 | 1203 | 1824 |
| 190 | 71228090V1 | 1215 | 1654 |
| 190 | 70857707V1 | 1220 | 1774 |
| 190 | 4625867H1 | 1221 | 1490 |
| 190 | 71225380V1 | 1274 | 1804 |
| 190 | 70858188V1 | 1290 | 1935 |
| 190 | 70858768V1 | 1303 | 1903 |
| 190 | 70864189V1 | 1307 | 1926 |
| 190 | 6911209H1 | 1323 | 1505 |
| 190 | 70858579V1 | 1330 | 1996 |
| 190 | 70858582V1 | 1366 | 2008 |
| 190 | 71224853V1 | 1396 | 1993 |
| 190 | 5640688H1 | 1416 | 1664 |
| 190 | 71226033V1 | 1421 | 2078 |
| 190 | 7359924H1 | 1437 | 2064 |
| 190 | 70861230V1 | 1450 | 2100 |
| 190 | 71226687V1 | 1457 | 2020 |
| 190 | 70864162V1 | 1463 | 2044 |
| 190 | 70855640V1 | 1491 | 2187 |
| 190 | 71225393V1 | 1507 | 2076 |
| 190 | 70858387V1 | 1539 | 2062 |
| 190 | 70855984V1 | 1520 | 1993 |
| 190 | 70857482V1 | 1520 | 2169 |
| 190 | 70855452V1 | 1526 | 2133 |
| 190 | 70861672V1 | 1541 | 2227 |
| 190 | 70861119V1 | 1548 | 2117 |
| 190 | 71225746V1 | 1558 | 2147 |
| 190 | 70856590V1 | 1568 | 2109 |
| 190 | 4030718F6 | 1573 | 1963 |
| 190 | 4030718H1 | 1573 | 1845 |
| 190 | 70856815V1 | 1611 | 2119 |
| 190 | 2742284H1 | 1626 | 1887 |
| 190 | 71228652V1 | 1651 | 2284 |
| 190 | 70855093V1 | 1689 | 2167 |
| 190 | 70863689V1 | 1722 | 2354 |
| 190 | 6412316H1 | 1730 | 1956 |
| 190 | 70857818V1 | 1740 | 2409 |
| 190 | 70860827V1 | 1745 | 2302 |
| 190 | 70864783V1 | 1755 | 2382 |
| 190 | 70863459V1 | 1752 | 2053 |
| 190 | 70858650V1 | 1822 | 2413 |
| 190 | 6911209J1 | 1830 | 2415 |
| 190 | 70146804V1 | 1869 | 2439 |
| 190 | 70146516V1 | 1869 | 2409 |
| 190 | 70863645V1 | 1877 | 2491 |
| 190 | 71225144V1 | 1889 | 2474 |
| 190 | 71224913V1 | 1929 | 2422 |
| 190 | 70146682V1 | 1936 | 2394 |
| 190 | 060994H1 | 1936 | 2131 |
| 190 | 70861917V1 | 1973 | 2563 |
| 190 | 2791792F6 | 2000 | 2315 |
| 190 | 2791792H1 | 2000 | 2297 |
| 190 | 70857602V1 | 2001 | 2402 |
| 190 | 71225364V1 | 2013 | 2660 |
| 190 | 70858585V1 | 2015 | 2639 |
| 190 | 70854964V1 | 2015 | 2644 |
| 190 | 70860984V1 | 2016 | 2573 |
| 190 | 70855603V1 | 2033 | 2704 |
| 190 | 70858486V1 | 2050 | 2280 |
| 190 | 70858723V1 | 2050 | 2280 |
| 190 | 70861733V1 | 2048 | 2785 |
| 190 | 70855437V1 | 2059 | 2683 |
| 190 | 70858374V1 | 2060 | 2596 |
| 190 | 449622H1 | 2069 | 2240 |
| 190 | 71228093V1 | 2070 | 2326 |
| 190 | 71227893V1 | 2070 | 2351 |
| 190 | 71227986V1 | 2072 | 2687 |
| 190 | 71225556V1 | 2086 | 2744 |
| 190 | 70864357V1 | 2095 | 2685 |
| 190 | 70855127V1 | 2110 | 2729 |
| 191 | 2106710R6 | 1204 | 1492 |
| 191 | 71032896V1 | 417 | 928 |
| 191 | 2004771H1 | 381 | 441 |
| 191 | 71134281V1 | 928 | 1489 |
| 191 | 71130579V1 | 1003 | 1490 |
| 191 | 71129556V1 | 1016 | 1488 |
| 191 | 71133640V1 | 1088 | 1509 |
| 191 | g3427925 | 591 | 1011 |
| 191 | 71133255V1 | 605 | 931 |
| 191 | 70643569V1 | 613 | 810 |
| 191 | 70639731V1 | 613 | 829 |
| 191 | 71133577V1 | 653 | 1365 |
| 191 | 71134103V1 | 657 | 1276 |
| 191 | 968289H1 | 143 | 416 |
| 191 | 7212584H1 | 176 | 467 |
| 191 | g4735242 | 212 | 473 |
| 191 | g4533052 | 255 | 473 |
| 191 | 2705278H1 | 1 | 210 |
| 191 | 2427258H1 | 8 | 163 |
| 191 | 991952H1 | 127 | 401 |
| 191 | 3070166H1 | 399 | 700 |
| 191 | 71131960V1 | 399 | 833 |
| 191 | 71130961V1 | 406 | 930 |
| 191 | 71133228V1 | 779 | 1472 |
| 191 | 2301872R6 | 503 | 813 |
| 191 | g1263124 | 505 | 671 |
| 191 | 1965957R6 | 512 | 843 |
| 191 | 6742448H1 | 398 | 474 |
| 191 | 6843692H1 | 413 | 692 |
| 191 | g3433649 | 563 | 1009 |
| 191 | 6883312H1 | 565 | 1039 |
| 191 | 70747238V1 | 585 | 712 |
| 191 | 1965957H1 | 512 | 685 |
| 191 | 2301872H1 | 512 | 686 |
| 191 | 6202014H1 | 534 | 931 |
| 191 | g4113683 | 562 | 1004 |
| 191 | 70749633V1 | 562 | 749 |
| 191 | 71129118V1 | 690 | 1233 |
| 191 | 5769401H1 | 682 | 1308 |
| 191 | g611097 | 808 | 1182 |
| 191 | 70641000V1 | 873 | 1297 |
| 191 | 2106710H1 | 1204 | 1488 |
| 191 | 2106710T6 | 1204 | 1448 |
| 191 | 861904H1 | 1343 | 1488 |
| 191 | 2709045F6 | 1441 | 1798 |
| 191 | 2709045H1 | 1442 | 1757 |
| 192 | 7345973H1 | 862 | 1457 |
| 192 | 7322101H1 | 921 | 1460 |
| 192 | 6850665H1 | 1366 | 1818 |
| 192 | 2654475F6 | 826 | 1310 |
| 192 | 2654475H1 | 826 | 1121 |
| 192 | g831331 | 1984 | 2353 |
| 192 | 1787542H1 | 2005 | 2223 |
| 192 | 6930205H1 | 2259 | 2725 |
| 192 | 4020229H1 | 1952 | 2228 |
| 192 | 6894957J1 | 1752 | 2259 |
| 192 | 4152694H1 | 1653 | 1911 |
| 192 | 3246645H1 | 1698 | 1866 |
| 192 | g4901556 | 1740 | 2205 |
| 192 | 2740754F6 | 2311 | 2784 |
| 192 | 2740754H1 | 2311 | 2564 |
| 192 | 1834220H1 | 2425 | 2687 |
| 192 | 4941620H1 | 2432 | 2695 |
| 192 | 4941983H1 | 2432 | 2711 |
| 192 | 2072075H1 | 2522 | 2772 |
| 192 | 5373319H1 | 2564 | 2779 |
| 192 | 3619907H1 | 2644 | 2921 |
| 192 | 4543335H1 | 2703 | 2966 |
| 192 | 6894957H1 | 2731 | 3286 |
| 192 | 4694271H1 | 2766 | 3009 |
| 192 | 4852731H1 | 2880 | 3138 |
| 192 | 2154473H1 | 2918 | 3190 |
| 192 | 2284114H1 | 2944 | 3172 |
| 192 | 4878579H1 | 2942 | 3086 |
| 192 | 4775770H1 | 2975 | 3235 |
| 192 | 2548439H1 | 3011 | 3245 |
| 192 | 2740754T6 | 3136 | 3642 |
| 192 | 7214289H1 | 3146 | 3371 |
| 192 | 3732858F6 | 3172 | 3639 |
| 192 | 3732858H1 | 3173 | 3398 |
| 192 | 4136158H1 | 3193 | 3478 |
| 192 | 2654475T6 | 3195 | 3653 |
| 192 | 3732858T6 | 3217 | 3639 |
| 192 | g4189007 | 3240 | 3680 |
| 192 | 856846H1 | 3327 | 3543 |
| 192 | g4076116 | 3329 | 3683 |
| 192 | g440877 | 1 | 3690 |
| 192 | g5544790 | 141 | 538 |
| 192 | 7106835H1 | 489 | 657 |
| 192 | 2313806H1 | 3487 | 3683 |
| 192 | 2841392H1 | 3499 | 3686 |
| 192 | 2097796H1 | 3545 | 3686 |
| 192 | 666777H1 | 3347 | 3550 |
| 192 | 815360H1 | 3387 | 3620 |
| 192 | 2104253H1 | 3439 | 3675 |
| 192 | g831278 | 3464 | 3690 |
| 193 | 5911845T6 | 1 | 432 |
| 193 | 5911845F8 | 1 | 588 |
| 193 | 5911845F6 | 1 | 484 |
| 193 | 5911845H1 | 1 | 254 |
| 193 | 5911845T8 | 22 | 443 |
| 194 | 5910555F8 | 1 | 587 |
| 194 | 5910555T8 | 85 | 489 |
| 194 | 5910555F6 | 1 | 640 |
| 194 | 5910555T6 | 1 | 575 |
| 194 | 5910555H1 | 1 | 194 |
| 195 | 6790675H1 | 1 | 380 |
| 195 | g1688992 | 67 | 154 |
| 195 | g4326739 | 68 | 512 |
| 196 | g274447 | 2556 | 2884 |
| 196 | g2051352 | 2558 | 2924 |
| 196 | g1859536 | 2558 | 2909 |
| 196 | g2817917 | 2567 | 2931 |
| 196 | g1783714 | 2570 | 2878 |
| 196 | g823824 | 2587 | 2923 |
| 196 | g2782889 | 2604 | 2887 |
| 196 | 4840554T6 | 2612 | 2903 |
| 196 | g565986 | 2639 | 2909 |
| 196 | g3277818 | 2645 | 2926 |
| 196 | g4149369 | 2649 | 2925 |
| 196 | 1575363H1 | 2664 | 2888 |
| 196 | 1575363F6 | 2664 | 2917 |
| 196 | 1575363T6 | 2666 | 2879 |
| 196 | g788188 | 2690 | 2909 |
| 196 | g1482370 | 2691 | 2910 |
| 196 | g864606 | 2696 | 2879 |
| 196 | 2006905H1 | 2712 | 2899 |
| 196 | 806183H1 | 2716 | 2867 |
| 196 | g842117 | 2730 | 2907 |
| 196 | g3742246 | 2738 | 2909 |
| 196 | 4882821H1 | 2771 | 3047 |
| 196 | 4884359H1 | 2771 | 3033 |
| 196 | 5090054H1 | 2781 | 3025 |
| 196 | 2277831T6 | 2784 | 2878 |
| 196 | 2343515H1 | 2792 | 2906 |
| 196 | 2343515F6 | 2792 | 2906 |
| 196 | g788214 | 2821 | 2899 |
| 196 | g1995403 | 2857 | 3182 |
| 196 | 5119623H1 | 2995 | 3277 |
| 196 | 3369954H1 | 3012 | 3294 |
| 196 | 5346406H1 | 3028 | 3177 |
| 196 | 2675533H1 | 3208 | 3394 |
| 196 | 3077845H1 | 3217 | 3451 |
| 196 | 5592052H1 | 3236 | 3495 |
| 196 | 996009H1 | 3251 | 3493 |
| 196 | 5041507H1 | 3296 | 3548 |
| 196 | 7032868H1 | 3301 | 3854 |
| 196 | 1665916H1 | 3358 | 3611 |
| 196 | 1665916F6 | 3358 | 3807 |
| 196 | 5513048H1 | 3393 | 3621 |
| 196 | 2969909H2 | 3439 | 3736 |
| 196 | 3978484H1 | 3473 | 3755 |
| 196 | g831015 | 3530 | 3895 |
| 196 | g821280 | 3561 | 3895 |
| 196 | g1885508 | 1 | 289 |
| 196 | g1110043 | 1 | 305 |
| 196 | 3349547F6 | 1 | 411 |
| 196 | 3349547H1 | 1 | 271 |
| 196 | 2741917H1 | 5 | 251 |
| 196 | 6991627H1 | 203 | 727 |
| 196 | 1434004H1 | 241 | 528 |
| 196 | 5587446H1 | 272 | 506 |
| 196 | g2880882 | 276 | 409 |
| 196 | 349280H1 | 375 | 614 |
| 196 | g713456 | 380 | 729 |
| 196 | g698683 | 403 | 727 |
| 196 | 6916554H1 | 603 | 1106 |
| 196 | 6719552H1 | 659 | 1266 |
| 196 | 4384945H1 | 704 | 965 |
| 196 | 4384979H1 | 704 | 882 |
| 196 | g1885551 | 910 | 1284 |
| 196 | 5019503H1 | 953 | 1210 |
| 196 | 4876805H1 | 996 | 1272 |
| 196 | 2479328H1 | 1055 | 1293 |
| 196 | 037774H1 | 1083 | 1306 |
| 196 | g1717439 | 1121 | 1557 |
| 196 | 4840554H1 | 1189 | 1453 |
| 196 | 4840554F6 | 1189 | 1633 |
| 196 | 3279387H1 | 1202 | 1456 |
| 196 | 5373713H1 | 1216 | 1468 |
| 196 | 4145650H1 | 1246 | 1331 |
| 196 | 4916687H1 | 1293 | 1450 |
| 196 | 4581558H1 | 1328 | 1543 |
| 196 | g2307061 | 1330 | 1763 |
| 196 | 4386045H1 | 1371 | 1651 |
| 196 | 4700744H1 | 1373 | 1635 |
| 196 | 6812975J1 | 1426 | 2021 |
| 196 | 6484322H1 | 1438 | 1623 |
| 196 | 1912429H1 | 1473 | 1693 |
| 196 | g1440342 | 1498 | 1770 |
| 196 | g4524899 | 1511 | 1896 |
| 196 | g768901 | 1513 | 1820 |
| 196 | g571029 | 1512 | 1840 |
| 196 | g880779 | 1513 | 1841 |
| 196 | g832097 | 1513 | 1884 |
| 196 | 4623266H1 | 1566 | 1825 |
| 196 | 3519427H1 | 1590 | 1955 |
| 196 | 3680478H1 | 1608 | 1840 |
| 196 | 6332293H1 | 1668 | 2034 |
| 196 | 4832174H1 | 1735 | 1965 |
| 196 | g1329683 | 1739 | 2321 |
| 196 | g787502 | 1779 | 2009 |
| 196 | g864605 | 1779 | 2053 |
| 196 | g787457 | 1779 | 2024 |
| 196 | g841915 | 1778 | 2047 |
| 196 | 5589418H1 | 1784 | 2030 |
| 196 | 5071250H1 | 1803 | 2062 |
| 196 | 3865563H1 | 1843 | 2217 |
| 196 | 1955962H1 | 1894 | 2151 |
| 196 | 4750673H2 | 1945 | 2009 |
| 196 | 442154H1 | 1966 | 2272 |
| 196 | 261197H1 | 1965 | 2281 |
| 196 | 444335H1 | 1966 | 2225 |
| 196 | 4515301H1 | 2028 | 2284 |
| 196 | 3780534H1 | 2055 | 2353 |
| 196 | 2191782F6 | 2063 | 2373 |
| 196 | 2191782H1 | 2063 | 2304 |
| 196 | 3735668H1 | 2072 | 2331 |
| 196 | 3038357H1 | 2117 | 2391 |
| 196 | 6812975H1 | 2124 | 2462 |
| 196 | 4633908H1 | 2138 | 2408 |
| 196 | 1339250F6 | 2140 | 2483 |
| 196 | 1339250H1 | 2140 | 2404 |
| 196 | 1339250T6 | 2142 | 2469 |
| 196 | 4324610H1 | 2150 | 2401 |
| 196 | 4266784H2 | 2156 | 2439 |
| 196 | 4834124H1 | 2174 | 2358 |
| 196 | g1783896 | 2175 | 2516 |
| 196 | g2207390 | 2175 | 2518 |
| 196 | 908344R2 | 2180 | 2516 |
| 196 | 908344H1 | 2180 | 2285 |
| 196 | 4748325H1 | 2199 | 2467 |
| 196 | 4746557H1 | 2200 | 2439 |
| 196 | g1859648 | 2231 | 2520 |
| 196 | 5565332H1 | 2244 | 2501 |
| 196 | g778446 | 2320 | 2632 |
| 196 | g831074 | 2319 | 2636 |
| 196 | g2053046 | 2330 | 2794 |
| 196 | 3031124H1 | 2353 | 2644 |
| 196 | 2277831H1 | 2391 | 2660 |
| 196 | 2277831R6 | 2391 | 2516 |
| 196 | g2552923 | 2437 | 2906 |
| 196 | g832055 | 2523 | 2923 |
| 196 | g1329627 | 2545 | 2922 |
| 196 | g4990052 | 2547 | 2834 |
| 196 | g4311136 | 2547 | 2993 |
| 196 | g4988520 | 2547 | 3007 |
| 196 | g4740301 | 2547 | 2833 |
| 196 | g3422370 | 2548 | 2923 |
| 196 | g4289096 | 2548 | 2904 |
| 196 | g3431513 | 2549 | 2876 |
| 196 | 000140H1 | 2555 | 2911 |
| 196 | 2543809H1 | 2555 | 2770 |
| 196 | 2435619H1 | 2555 | 2713 |
| 196 | g5113593 | 2555 | 2917 |
| 196 | g4189560 | 2555 | 2877 |
| 196 | g3108551 | 2555 | 2916 |
| 196 | g1476749 | 2557 | 2878 |
| 196 | g5112875 | 2555 | 2909 |
| 196 | g4565709 | 2555 | 2921 |
| 196 | g3280248 | 2555 | 2910 |
| 196 | g2208127 | 2555 | 2878 |
| 197 | 5965475H1 | 1 | 564 |
| 197 | g1162029 | 1 | 146 |
| 197 | g1648409 | 1 | 322 |
| 197 | 5833130H1 | 1 | 266 |
| 197 | 6151063H1 | 53 | 353 |
| 198 | 6764201J1 | 1 | 585 |
| 198 | 986476R6 | 5 | 479 |
| 198 | 986476H1 | 5 | 303 |
| 198 | 4180212H1 | 8 | 260 |
| 198 | g3933038 | 75 | 566 |
| 198 | 4029248H1 | 122 | 377 |
| 198 | 4029227H1 | 122 | 370 |
| 199 | g4196260 | 1 | 320 |
| 199 | g2218495 | 1 | 313 |
| 199 | 3685061H1 | 1 | 293 |
| 199 | 6821713J1 | 1 | 518 |
| 199 | 5372078H1 | 25 | 226 |
| 199 | 4205860H1 | 169 | 437 |
| 199 | 4205654H1 | 169 | 416 |
| 200 | 6868907H1 | 1 | 542 |
| 200 | 1543453R6 | 24 | 440 |
| 200 | 1543453H1 | 24 | 217 |
| 200 | 5055563H1 | 38 | 307 |
| 200 | 5055563F9 | 38 | 607 |
| 200 | 4778273F8 | 126 | 752 |
| 200 | 4778273H1 | 126 | 397 |
| 200 | 2724784H1 | 137 | 372 |
| 200 | 2925738H1 | 339 | 605 |
| 200 | 4778273T9 | 559 | 1136 |
| 200 | 1543453T6 | 610 | 1221 |
| 200 | 1921258H1 | 832 | 1046 |
| 200 | 1921258F6 | 833 | 1233 |
| 201 | g4664146 | 1 | 478 |
| 201 | g6140998 | 2 | 458 |
| 201 | g6144190 | 2 | 422 |
| 201 | g6139959 | 2 | 400 |
| 201 | 6764092J1 | 10 | 589 |
| 201 | g2910438 | 59 | 537 |
| 201 | 4570842H1 | 67 | 315 |
| 201 | 4921180H1 | 69 | 331 |
| 201 | 5955592H1 | 86 | 596 |
| 201 | g2904695 | 89 | 474 |
| 201 | g2841122 | 108 | 375 |
| 201 | 6982639H1 | 384 | 913 |
| 201 | 1570643T6 | 422 | 944 |
| 201 | g5232046 | 430 | 870 |
| 201 | 7377338H1 | 537 | 1127 |
| 201 | 1570643F6 | 661 | 972 |
| 201 | 3119826H1 | 732 | 1002 |
| 201 | 6097252H1 | 753 | 954 |
| 201 | 1570316H1 | 810 | 972 |
| 201 | 1570643H1 | 814 | 972 |
| 202 | 5913683H1 | 1 | 281 |
| 202 | 5320619F9 | 1 | 372 |
| 202 | 5913683F6 | 4 | 358 |
| 202 | 5321234F9 | 6 | 484 |
| 202 | 5914061H1 | 6 | 264 |
| 202 | 5914061F8 | 6 | 444 |
| 202 | 5913683T6 | 28 | 431 |
| 202 | 5913683F8 | 27 | 200 |
| 202 | 6269343H1 | 36 | 444 |
| 202 | 6269670H1 | 59 | 444 |
| 203 | 71035493V1 | 961 | 1616 |
| 203 | 5771773H1 | 971 | 1635 |
| 203 | 5952931H1 | 971 | 1230 |
| 203 | 5989634H1 | 968 | 1168 |
| 203 | 5800985H1 | 971 | 1270 |
| 203 | 5800543H1 | 976 | 1477 |
| 203 | 5805534H1 | 976 | 1299 |
| 203 | 5831382H2 | 976 | 1193 |
| 203 | 6166604H1 | 984 | 1567 |
| 203 | 6153585H1 | 982 | 1221 |
| 203 | 5804655H1 | 1003 | 1323 |
| 203 | 7075719H1 | 1022 | 1600 |
| 203 | 5798865H1 | 1027 | 1604 |
| 203 | 6964153H1 | 1031 | 1629 |
| 203 | 6183568H1 | 1031 | 1322 |
| 203 | 6183681H1 | 1031 | 1308 |
| 203 | 71001091V1 | 1050 | 1610 |
| 203 | 6217476H1 | 1061 | 1617 |
| 203 | 6148902H1 | 1068 | 1615 |
| 203 | 6114310H1 | 1078 | 1353 |
| 203 | 5806522H1 | 1090 | 1418 |
| 203 | 6146547H1 | 1093 | 1723 |
| 203 | 7220801H1 | 1094 | 1676 |
| 203 | 6132328H1 | 1094 | 1386 |
| 203 | 5995785H1 | 1095 | 1395 |
| 203 | 7322507H1 | 1099 | 1470 |
| 203 | 6113383H1 | 1100 | 1461 |
| 203 | 5975102H1 | 1102 | 1685 |
| 203 | 6148050H1 | 1102 | 1633 |
| 203 | 5977862H1 | 1102 | 1618 |
| 203 | 6038505H1 | 1104 | 1743 |
| 203 | 7080569H1 | 1103 | 1454 |
| 203 | 6116490H1 | 1102 | 1394 |
| 203 | 71360704V1 | 1105 | 1709 |
| 203 | 5892681H1 | 1104 | 1351 |
| 203 | 6164075H1 | 1107 | 1725 |
| 203 | 6181324H1 | 1107 | 1428 |
| 203 | 5770720H1 | 1114 | 1752 |
| 203 | 5797766H1 | 1114 | 1712 |
| 203 | 5826374H1 | 1118 | 1687 |
| 203 | 5796423H1 | 1114 | 1401 |
| 203 | 6036824H1 | 1117 | 1719 |
| 203 | 6034194H1 | 1117 | 1678 |
| 203 | 5769794H1 | 1118 | 1656 |
| 203 | 6134213H1 | 1117 | 1460 |
| 203 | 5770890H1 | 1122 | 1760 |
| 203 | 5976811H1 | 1134 | 1770 |
| 203 | 6178910H1 | 1139 | 1429 |
| 203 | 5975668H1 | 1145 | 1778 |
| 203 | 5975687H1 | 1145 | 1729 |
| 203 | 6214924H1 | 1146 | 1720 |
| 203 | 6183602H1 | 1162 | 1505 |
| 203 | 6183689H1 | 1162 | 1461 |
| 203 | 5803566H1 | 1165 | 1465 |
| 203 | 6040276H1 | 1169 | 1782 |
| 203 | 5975156H1 | 1169 | 1687 |
| 203 | 5973594H1 | 1169 | 1675 |
| 203 | 5995226H1 | 1169 | 1499 |
| 203 | 5993875H1 | 1170 | 1487 |
| 203 | 6078894H1 | 1169 | 1465 |
| 203 | 5825616H1 | 1173 | 1541 |
| 203 | 5921387T8 | 1177 | 1760 |
| 203 | 6042666H1 | 1187 | 1479 |
| 203 | 7185125H1 | 1194 | 1778 |
| 203 | 5993778H1 | 1197 | 1463 |
| 203 | 5993775H1 | 1193 | 1513 |
| 203 | 6168554H1 | 1198 | 1502 |
| 203 | 60047572D4 | 1199 | 1757 |
| 203 | 5824125H1 | 1200 | 1655 |
| 203 | 5994991H1 | 1200 | 1515 |
| 203 | 6112929H1 | 1204 | 1502 |
| 203 | 7330358H1 | 1219 | 1909 |
| 203 | 5768301H1 | 1220 | 1806 |
| 203 | 5769260H1 | 1218 | 1756 |
| 203 | 7330481H1 | 1221 | 1868 |
| 203 | 5995163H1 | 1233 | 1569 |
| 203 | 6959960H1 | 1236 | 1747 |
| 203 | 60206936U1 | 1239 | 1912 |
| 203 | 6034683H1 | 1243 | 1803 |
| 203 | 6169613H1 | 1244 | 1582 |
| 203 | 4294314T9 | 1258 | 1713 |
| 203 | 6041063H1 | 1248 | 1905 |
| 203 | 71035949V1 | 1252 | 1885 |
| 203 | 5952448H1 | 1256 | 1571 |
| 203 | 5974564H1 | 1254 | 1859 |
| 203 | 5991486H1 | 1255 | 1599 |
| 203 | 7264840H1 | 1257 | 1915 |
| 203 | 5991328H1 | 1255 | 1611 |
| 203 | 6112291H1 | 1256 | 1594 |
| 203 | 7080084H1 | 1261 | 1885 |
| 203 | 5771870H1 | 1263 | 1827 |
| 203 | 6170079H1 | 1262 | 1582 |
| 203 | 6080872H1 | 1264 | 1862 |
| 203 | 5824738H1 | 1269 | 1837 |
| 203 | 3390106T6 | 1274 | 1662 |
| 203 | 60221621V1 | 1274 | 1677 |
| 203 | 7004345H1 | 1284 | 1909 |
| 203 | 60109238B1 | 1286 | 1912 |
| 203 | 7186303H1 | 1290 | 1881 |
| 203 | 7025213H1 | 1297 | 1900 |
| 203 | 7182261H1 | 1299 | 1807 |
| 203 | 7193486H2 | 1306 | 1929 |
| 203 | 6736012H1 | 1319 | 1907 |
| 203 | 5970654H1 | 1322 | 1912 |
| 203 | 5770017H1 | 1327 | 1919 |
| 203 | 5802957H1 | 1327 | 1561 |
| 203 | 5800770H1 | 1328 | 1601 |
| 203 | 5804521H1 | 1331 | 1550 |
| 203 | 5995788H1 | 1330 | 1639 |
| 203 | 7331927H1 | 1332 | 1935 |
| 203 | 5769517H1 | 1333 | 1918 |
| 203 | 5967562H1 | 1333 | 1856 |
| 203 | 6163438H1 | 1337 | 1896 |
| 203 | 5953896H1 | 1338 | 1640 |
| 203 | 5789044H1 | 1338 | 1633 |
| 203 | 5975734H1 | 1339 | 1916 |
| 203 | 6078714H1 | 1338 | 1668 |
| 203 | 5786342H1 | 1338 | 1646 |
| 203 | 6175953H1 | 1340 | 1636 |
| 203 | 5805592H1 | 1340 | 1639 |
| 203 | 5952736H1 | 1351 | 1684 |
| 203 | 3693154F6 | 1354 | 1935 |
| 203 | 6217933H1 | 1356 | 1756 |
| 203 | 7160128H1 | 628 | 1197 |
| 203 | 6117709H1 | 630 | 956 |
| 203 | 6180232H1 | 632 | 859 |
| 203 | 6173095H1 | 645 | 974 |
| 203 | 6132472H1 | 645 | 956 |
| 203 | 6150854H1 | 647 | 996 |
| 203 | 5804474H1 | 647 | 944 |
| 203 | 6043158H1 | 647 | 922 |
| 203 | 6150959H1 | 647 | 898 |
| 203 | 6162572H1 | 648 | 1211 |
| 203 | 5803707H1 | 648 | 950 |
| 203 | 6111936H1 | 653 | 955 |
| 203 | 6111235H1 | 656 | 969 |
| 203 | 5953419H1 | 659 | 1020 |
| 203 | 5995735H1 | 659 | 974 |
| 203 | 6002288H1 | 659 | 917 |
| 203 | 6144013H1 | 664 | 1176 |
| 203 | 6166779H1 | 664 | 1176 |
| 203 | 6052294H1 | 666 | 889 |
| 203 | 6053478J1 | 664 | 1250 |
| 203 | 6052294J1 | 666 | 1098 |
| 203 | 5995222H1 | 671 | 1001 |
| 203 | 5990146H1 | 671 | 994 |
| 203 | 6079085H1 | 671 | 956 |
| 203 | 5798802H1 | 672 | 1277 |
| 203 | 5975323H1 | 681 | 1238 |
| 203 | 7327981H2 | 684 | 1173 |
| 203 | 6155086H1 | 691 | 1046 |
| 203 | 6183751H1 | 699 | 1001 |
| 203 | 6183830H1 | 699 | 983 |
| 203 | 6215696H1 | 701 | 1298 |
| 203 | 6109481H1 | 701 | 1023 |
| 203 | 6030242H2 | 702 | 1001 |
| 203 | 6163760H1 | 706 | 1074 |
| 203 | 6153438H1 | 710 | 1236 |
| 203 | 6111179H1 | 710 | 982 |
| 203 | 6116630H1 | 723 | 1044 |
| 203 | 6151576H1 | 726 | 998 |
| 203 | 6111753H1 | 732 | 1001 |
| 203 | 5824855H1 | 736 | 1199 |
| 203 | 5991422H1 | 740 | 1085 |
| 203 | 6180759H1 | 740 | 1069 |
| 203 | 6135676H1 | 741 | 1031 |
| 203 | 5954296H1 | 741 | 983 |
| 203 | 6154930H1 | 741 | 1095 |
| 203 | 6145090H1 | 747 | 1336 |
| 203 | 7215869H1 | 750 | 1359 |
| 203 | 6110012H1 | 754 | 1069 |
| 203 | 6135068H1 | 757 | 1061 |
| 203 | 6035941H1 | 759 | 1259 |
| 203 | 6163478H1 | 764 | 1336 |
| 203 | 5968127H1 | 760 | 1255 |
| 203 | 6178490H1 | 763 | 1018 |
| 203 | 60204862U1 | 771 | 1320 |
| 203 | 6108986H1 | 773 | 1083 |
| 203 | 6097994H1 | 787 | 1118 |
| 203 | 6165174H1 | 793 | 1081 |
| 203 | 6115201H1 | 792 | 1129 |
| 203 | 5798369H1 | 797 | 1419 |
| 203 | 5801102H1 | 797 | 1105 |
| 203 | 6112951H1 | 797 | 997 |
| 203 | 6109295H1 | 803 | 1129 |
| 203 | 6110881H1 | 805 | 1081 |
| 203 | 6179893H1 | 804 | 1105 |
| 203 | 5955376H1 | 806 | 1170 |
| 203 | 7359669H1 | 815 | 1090 |
| 203 | 6176408H1 | 813 | 1095 |
| 203 | 7004683H1 | 820 | 1427 |
| 203 | 7004582H1 | 820 | 1396 |
| 203 | 71033163V1 | 820 | 1364 |
| 203 | 5974746H1 | 827 | 1459 |
| 203 | 7325468H1 | 829 | 1480 |
| 203 | 6035163H1 | 839 | 1457 |
| 203 | 6112093H1 | 835 | 1148 |
| 203 | 6177785H1 | 836 | 1124 |
| 203 | 5798782H1 | 836 | 917 |
| 203 | 6150235H1 | 848 | 1459 |
| 203 | 5952964H1 | 849 | 1130 |
| 203 | 6218804H1 | 851 | 1196 |
| 203 | 5804744H1 | 853 | 1162 |
| 203 | 5771556H1 | 858 | 1477 |
| 203 | 5994894H1 | 856 | 1166 |
| 203 | 71036246V1 | 861 | 1368 |
| 203 | 5915476H1 | 858 | 1096 |
| 203 | 6163363H1 | 863 | 1499 |
| 203 | 5796628H1 | 870 | 1046 |
| 203 | 6115353H1 | 872 | 1144 |
| 203 | 6115444H1 | 876 | 1097 |
| 203 | 6033285H1 | 884 | 1516 |
| 203 | 6145417H1 | 890 | 1443 |
| 203 | 6172878H1 | 890 | 1197 |
| 203 | 6172790H1 | 890 | 1176 |
| 203 | 5801236H1 | 902 | 1168 |
| 203 | 6113354H1 | 906 | 1275 |
| 203 | 6032732H1 | 906 | 1459 |
| 203 | 6130142H1 | 908 | 1245 |
| 203 | 6168522H1 | 913 | 1264 |
| 203 | 6037518H1 | 915 | 1525 |
| 203 | 6032546H1 | 914 | 1542 |
| 203 | 60104786D4 | 912 | 1222 |
| 203 | 6176164H1 | 924 | 1214 |
| 203 | 6133149H1 | 913 | 1026 |
| 203 | 7320972H1 | 920 | 1515 |
| 203 | 6033149H1 | 923 | 1523 |
| 203 | 5970892H1 | 924 | 1548 |
| 203 | 6036731H1 | 925 | 1529 |
| 203 | 5970884H1 | 924 | 1510 |
| 203 | 6113366H1 | 923 | 1026 |
| 203 | 7185073H1 | 932 | 1524 |
| 203 | 5827058H1 | 935 | 988 |
| 203 | 5770524H1 | 940 | 1589 |
| 203 | 6114346H1 | 936 | 1210 |
| 203 | 5990721H1 | 942 | 1263 |
| 203 | 5975145H1 | 947 | 1489 |
| 203 | 6044290H1 | 941 | 1226 |
| 203 | 5768644H1 | 957 | 1532 |
| 203 | 5971326H1 | 948 | 1554 |
| 203 | 6110988H1 | 946 | 1173 |
| 203 | 6099321H1 | 948 | 1236 |
| 203 | 6173091H1 | 62 | 390 |
| 203 | 6036155H1 | 65 | 613 |
| 203 | 6180780H1 | 65 | 387 |
| 203 | 5770305H1 | 66 | 615 |
| 203 | 7215805H1 | 67 | 610 |
| 203 | 7024381H1 | 67 | 430 |
| 203 | 6107796H1 | 67 | 255 |
| 203 | 7221886H1 | 69 | 646 |
| 203 | 5769589H1 | 70 | 647 |
| 203 | 7328380H1 | 73 | 643 |
| 203 | 4571391F8 | 75 | 628 |
| 203 | 5771842H1 | 79 | 676 |
| 203 | 6108158H1 | 79 | 418 |
| 203 | 6176812H1 | 79 | 395 |
| 203 | 6177958H1 | 79 | 393 |
| 203 | 6179512H1 | 79 | 393 |
| 203 | 6181431H1 | 79 | 384 |
| 203 | 7008218H1 | 79 | 694 |
| 203 | 6215727H1 | 79 | 590 |
| 203 | 6151732H1 | 84 | 317 |
| 203 | 5973312H1 | 84 | 615 |
| 203 | 60126922U1 | 86 | 662 |
| 203 | 5974449H1 | 88 | 675 |
| 203 | 6111184H1 | 90 | 170 |
| 203 | 6113115H1 | 90 | 429 |
| 203 | 6000095H1 | 91 | 474 |
| 203 | 6182051H1 | 91 | 394 |
| 203 | 7272693H1 | 91 | 677 |
| 203 | 7385648H1 | 96 | 287 |
| 203 | 6135054H1 | 99 | 403 |
| 203 | 6182449H1 | 104 | 467 |
| 203 | 5775693H1 | 104 | 719 |
| 203 | 6175825H1 | 106 | 381 |
| 203 | 5968167H1 | 118 | 728 |
| 203 | 60120598D1 | 108 | 278 |
| 203 | 6147691H1 | 122 | 616 |
| 203 | 6115311H1 | 133 | 425 |
| 203 | 5975411H1 | 137 | 630 |
| 203 | 5915392H1 | 144 | 416 |
| 203 | 6130193H1 | 148 | 465 |
| 203 | 7328558H1 | 147 | 830 |
| 203 | 5996192H1 | 166 | 467 |
| 203 | 5977403H1 | 168 | 658 |
| 203 | 5989334H1 | 177 | 480 |
| 203 | 6109928H1 | 179 | 509 |
| 203 | 6172501H1 | 186 | 392 |
| 203 | 6172633H1 | 186 | 479 |
| 203 | 7183712H1 | 187 | 769 |
| 203 | 7025064H1 | 194 | 618 |
| 203 | 7325279H1 | 201 | 812 |
| 203 | 6116494H1 | 204 | 493 |
| 203 | 5953527H1 | 210 | 543 |
| 203 | 5892842H1 | 214 | 481 |
| 203 | 5993811H1 | 218 | 546 |
| 203 | 6109818H1 | 226 | 448 |
| 203 | 6044864H1 | 228 | 751 |
| 203 | 6182368H1 | 242 | 578 |
| 203 | 7326560H2 | 250 | 893 |
| 203 | 6033805H1 | 253 | 837 |
| 203 | 6101869H1 | 257 | 545 |
| 203 | 6170505H1 | 265 | 596 |
| 203 | 6152125H1 | 265 | 596 |
| 203 | 6137458H1 | 265 | 568 |
| 203 | 5958352H1 | 265 | 856 |
| 203 | 5975525H1 | 294 | 899 |
| 203 | 5838053H1 | 296 | 585 |
| 203 | 5796171H1 | 304 | 858 |
| 203 | 6112187H1 | 310 | 639 |
| 203 | 6165284H1 | 313 | 858 |
| 203 | 5826529H1 | 320 | 764 |
| 203 | 6172048H1 | 336 | 644 |
| 203 | 6035764H1 | 326 | 932 |
| 203 | 6966778H1 | 329 | 425 |
| 203 | 6172080H1 | 328 | 576 |
| 203 | 7182401H1 | 354 | 954 |
| 203 | 7269240H1 | 357 | 994 |
| 203 | 6029089H1 | 360 | 667 |
| 203 | 6100553H1 | 367 | 674 |
| 203 | 5825841H1 | 387 | 1002 |
| 203 | 7214023H1 | 396 | 923 |
| 203 | 6109604H1 | 393 | 729 |
| 203 | 6143986H1 | 411 | 1031 |
| 203 | 6117765H1 | 421 | 543 |
| 203 | 6136677H1 | 423 | 744 |
| 203 | 6041789H1 | 430 | 1047 |
| 203 | 5989594H1 | 448 | 730 |
| 203 | 6053478H1 | 452 | 1086 |
| 203 | 6109628H1 | 453 | 711 |
| 203 | 6044864J1 | 455 | 1098 |
| 203 | 7180879H1 | 457 | 1063 |
| 203 | 5797850H1 | 461 | 930 |
| 203 | 6115841H1 | 461 | 756 |
| 203 | 6137438H1 | 468 | 772 |
| 203 | 6731970H1 | 474 | 1055 |
| 203 | 6108268H1 | 478 | 793 |
| 203 | 6115462H1 | 491 | 718 |
| 203 | 5891369H1 | 517 | 775 |
| 203 | 6001442H1 | 524 | 1096 |
| 203 | 7360854H1 | 525 | 694 |
| 203 | 5974453H1 | 539 | 1087 |
| 203 | 6109032H1 | 534 | 897 |
| 203 | 5892176H1 | 534 | 868 |
| 203 | 5953990H1 | 534 | 804 |
| 203 | 5831262H1 | 534 | 726 |
| 203 | 5955474H1 | 543 | 886 |
| 203 | 5955458H1 | 543 | 888 |
| 203 | 6111439H1 | 544 | 769 |
| 203 | 6111316H1 | 546 | 881 |
| 203 | 5797137H1 | 577 | 1094 |
| 203 | 6133207H1 | 586 | 897 |
| 203 | 7328606H1 | 589 | 1133 |
| 203 | 7261854H1 | 590 | 1225 |
| 203 | 6100514H1 | 596 | 898 |
| 203 | 6040074H1 | 599 | 1199 |
| 203 | 5824550H1 | 606 | 1060 |
| 203 | 2814920F7 | 612 | 1092 |
| 203 | 6181437H1 | 617 | 905 |
| 203 | 5771704H1 | 626 | 1260 |
| 203 | 6131361H1 | 624 | 937 |
| 203 | 71216557V1 | 628 | 1188 |
| 203 | 6141011H1 | 1 | 156 |
| 203 | 6108212H1 | 1 | 324 |
| 203 | 6177861H1 | 2 | 301 |
| 203 | 5976933H1 | 12 | 479 |
| 203 | 7024610H1 | 39 | 249 |
| 203 | 5973147H1 | 35 | 622 |
| 203 | 6735535H1 | 39 | 221 |
| 203 | 7279008H1 | 39 | 531 |
| 203 | 60264017D1 | 32 | 569 |
| 203 | 7079629H2 | 44 | 358 |
| 203 | 5972573H1 | 41 | 686 |
| 203 | 7160586H1 | 34 | 619 |
| 203 | 6784013H1 | 35 | 645 |
| 203 | 7083178H1 | 36 | 637 |
| 203 | 7182216H1 | 36 | 592 |
| 203 | 7184701H1 | 36 | 578 |
| 203 | 6170753H1 | 36 | 341 |
| 203 | 7261837H1 | 36 | 644 |
| 203 | 6180976H1 | 37 | 372 |
| 203 | 6170740H1 | 37 | 340 |
| 203 | 7262607H1 | 39 | 648 |
| 203 | 7220667H1 | 38 | 636 |
| 203 | 7182859H1 | 39 | 616 |
| 203 | 6099372H1 | 39 | 324 |
| 203 | 7260783H1 | 39 | 675 |
| 203 | 7316234H1 | 39 | 709 |
| 203 | 4625563F9 | 39 | 673 |
| 203 | 7160372H1 | 39 | 643 |
| 203 | 7160758H1 | 39 | 615 |
| 203 | 7216608H1 | 39 | 623 |
| 203 | 7219614H1 | 39 | 613 |
| 203 | 7213493H1 | 39 | 643 |
| 203 | 7160235H1 | 39 | 555 |
| 203 | 7317368H1 | 39 | 420 |
| 203 | 6097675H1 | 39 | 380 |
| 203 | 6098127H1 | 39 | 375 |
| 203 | 6171834H1 | 39 | 358 |
| 203 | 6169216H1 | 39 | 372 |
| 203 | 6131659H1 | 39 | 358 |
| 203 | 6177913H1 | 39 | 350 |
| 203 | 6109871H1 | 39 | 347 |
| 203 | 6132882H1 | 39 | 346 |
| 203 | 6136114H1 | 39 | 340 |
| 203 | 6132559H1 | 39 | 328 |
| 203 | 5989784H1 | 39 | 328 |
| 203 | 6135227H1 | 39 | 339 |
| 203 | 6028575H1 | 39 | 267 |
| 203 | 5914782H1 | 57 | 278 |
| 203 | 7359725H1 | 39 | 685 |
| 203 | 6084122H1 | 40 | 673 |
| 203 | 7083662H1 | 39 | 685 |
| 203 | 7358734H1 | 39 | 682 |
| 203 | 7181284H1 | 39 | 557 |
| 203 | 60264004D1 | 39 | 552 |
| 203 | 6080769H1 | 50 | 645 |
| 203 | 7212965H1 | 39 | 444 |
| 203 | 6174304H1 | 39 | 387 |
| 203 | 6117564H1 | 40 | 363 |
| 203 | 6132067H1 | 39 | 350 |
| 203 | 6099630H1 | 39 | 322 |
| 203 | 6134055H1 | 62 | 379 |
| 203 | 5994380H1 | 42 | 384 |
| 203 | 6098224H1 | 42 | 362 |
| 203 | 6132993H1 | 42 | 337 |
| 203 | 7213881H1 | 43 | 644 |
| 203 | 6081567H1 | 43 | 636 |
| 203 | 6132164H1 | 43 | 385 |
| 203 | 6101109H1 | 43 | 377 |
| 203 | 5992072H1 | 43 | 372 |
| 203 | 6078760H1 | 43 | 365 |
| 203 | 5995191H1 | 43 | 374 |
| 203 | 6171974H1 | 43 | 359 |
| 203 | 5989890H1 | 43 | 350 |
| 203 | 6181359H1 | 44 | 353 |
| 203 | 6110331H1 | 43 | 347 |
| 203 | 6111434H1 | 43 | 339 |
| 203 | 6168435H1 | 43 | 284 |
| 203 | 7268992H1 | 45 | 675 |
| 203 | 6081557H1 | 44 | 614 |
| 203 | 6110567H1 | 43 | 283 |
| 203 | 7272829H1 | 45 | 685 |
| 203 | 5992910H1 | 45 | 388 |
| 203 | 6131653H1 | 47 | 373 |
| 203 | 5796567H1 | 45 | 109 |
| 203 | 7263260H1 | 46 | 647 |
| 203 | 7158311H1 | 46 | 588 |
| 203 | 6097721H1 | 47 | 393 |
| 203 | 6136962H1 | 46 | 404 |
| 203 | 6170754H1 | 47 | 353 |
| 203 | 6107830H1 | 47 | 235 |
| 203 | 7275183H1 | 48 | 644 |
| 203 | 7182041H1 | 49 | 616 |
| 203 | 7182971H1 | 49 | 616 |
| 203 | 5991415H1 | 48 | 388 |
| 203 | 6112705H1 | 48 | 373 |
| 203 | 6098861H1 | 48 | 347 |
| 203 | 6099983H1 | 48 | 243 |
| 203 | 7283631H1 | 49 | 718 |
| 203 | 6039521H1 | 49 | 650 |
| 203 | 5992224H1 | 49 | 375 |
| 203 | 6170022H1 | 49 | 387 |
| 203 | 5992273H1 | 49 | 363 |
| 203 | 6114538H1 | 48 | 348 |
| 203 | 7272102H1 | 50 | 635 |
| 203 | 6136863H1 | 50 | 389 |
| 203 | 6085163H1 | 68 | 331 |
| 203 | 6115314H1 | 50 | 306 |
| 203 | 7270954H1 | 51 | 687 |
| 203 | 7190289H2 | 51 | 619 |
| 203 | 4624253F8 | 54 | 733 |
| 203 | 6037863H1 | 54 | 590 |
| 203 | 5973394H1 | 54 | 630 |
| 203 | 6168342H1 | 54 | 412 |
| 203 | 6733426H1 | 73 | 419 |
| 203 | 5770643H1 | 55 | 644 |
| 203 | 6733166H1 | 54 | 647 |
| 203 | 7321688H1 | 58 | 593 |
| 203 | 6038935H1 | 58 | 635 |
| 203 | 6134077H1 | 56 | 399 |
| 203 | 7185189H1 | 56 | 643 |
| 203 | 6034778H1 | 59 | 683 |
| 203 | 7330333H1 | 58 | 524 |
| 203 | 7184720H1 | 62 | 640 |
| 203 | 60104786B2 | 1354 | 1910 |
| 203 | 6177422H1 | 1353 | 1657 |
| 203 | 5824565H1 | 1360 | 1894 |
| 203 | 6110675H1 | 1364 | 1676 |
| 203 | 6042367H1 | 1364 | 1661 |
| 203 | 6035204H1 | 1366 | 1929 |
| 203 | 6170972H1 | 1365 | 1682 |
| 203 | 6181601H1 | 1365 | 1667 |
| 203 | 5802968H1 | 1367 | 1676 |
| 203 | 4362784F8 | 1372 | 1927 |
| 203 | 5996082H1 | 1377 | 1699 |
| 203 | 5996003H1 | 1375 | 1666 |
| 203 | 5995203H1 | 1382 | 1721 |
| 203 | 6133437H1 | 1383 | 1700 |
| 203 | 6178414H1 | 1393 | 1699 |
| 203 | 6179001H1 | 1401 | 1548 |
| 203 | 5769429H1 | 1403 | 1918 |
| 203 | 5785086H1 | 1404 | 1722 |
| 203 | 5801902H1 | 1405 | 1725 |
| 203 | 5792833H1 | 1404 | 1477 |
| 203 | 5949501H1 | 1415 | 1784 |
| 203 | g6228962 | 1428 | 1937 |
| 203 | g6228709 | 1432 | 1938 |
| 203 | g6300732 | 1431 | 1938 |
| 203 | 60126922B1 | 1434 | 1887 |
| 203 | 6173439H1 | 1434 | 1747 |
| 203 | 5769804H1 | 1434 | 1920 |
| 203 | g6301637 | 1436 | 1938 |
| 203 | g6302015 | 1437 | 1936 |
| 203 | 5953730H1 | 1440 | 1530 |
| 203 | 5770049H1 | 1440 | 1927 |
| 203 | 7082145H1 | 1440 | 1907 |
| 203 | g6301063 | 1443 | 1936 |
| 203 | 6133907H1 | 1444 | 1778 |
| 203 | g6039672 | 1445 | 1932 |
| 203 | g6301561 | 1453 | 1938 |
| 203 | 5990839H1 | 1456 | 1799 |
| 203 | 5768633H1 | 1459 | 1920 |
| 203 | g6229211 | 1555 | 1933 |
| 203 | g6132479 | 1555 | 1933 |
| 203 | g6030521 | 1555 | 1935 |
| 203 | g6197062 | 1561 | 1937 |
| 203 | 6152884H1 | 1564 | 1869 |
| 203 | g6471957 | 1565 | 1928 |
| 203 | 5955189H1 | 1566 | 1854 |
| 203 | 6030104H2 | 1566 | 1861 |
| 203 | 5805947H1 | 1565 | 1840 |
| 203 | 5771690H1 | 1566 | 1928 |
| 203 | g6030549 | 1568 | 1928 |
| 203 | g6198562 | 1567 | 1928 |
| 203 | 6097965H1 | 1568 | 1889 |
| 203 | 6151677H1 | 1571 | 1866 |
| 203 | 5974518H1 | 1571 | 1926 |
| 203 | g6229210 | 1574 | 1933 |
| 203 | 6028702H1 | 1579 | 1813 |
| 203 | g6470581 | 1580 | 1920 |
| 203 | g6198908 | 1587 | 1928 |
| 203 | g6044672 | 1588 | 1928 |
| 203 | g6470803 | 1511 | 1930 |
| 203 | g6027906 | 1513 | 1934 |
| 203 | g6074740 | 1524 | 1928 |
| 203 | g6026164 | 1523 | 1928 |
| 203 | g6133768 | 1528 | 1932 |
| 203 | 6179968H1 | 1526 | 1784 |
| 203 | 5991468H1 | 1528 | 1863 |
| 203 | 5786634H1 | 1530 | 1832 |
| 203 | 5786701H1 | 1530 | 1715 |
| 203 | 5786734H1 | 1532 | 1652 |
| 203 | g6047869 | 1534 | 1921 |
| 203 | 6176458H1 | 1547 | 1825 |
| 203 | 5816770H1 | 1550 | 1883 |
| 203 | 5817968H1 | 1550 | 1746 |
| 203 | g6471065 | 1550 | 1928 |
| 203 | 5817174H1 | 1550 | 1832 |
| 203 | g6471718 | 1552 | 1937 |
| 203 | g6464005 | 1552 | 1937 |
| 203 | 6108818H1 | 1554 | 1787 |
| 203 | 7181625H1 | 1553 | 1920 |
| 203 | g6043984 | 1488 | 1938 |
| 203 | g6476041 | 1488 | 1930 |
| 203 | g6075390 | 1489 | 1937 |
| 203 | g6028910 | 1490 | 1937 |
| 203 | g6073495 | 1490 | 1930 |
| 203 | g6401184 | 1490 | 1930 |
| 203 | g6073672 | 1490 | 1937 |
| 203 | g6036791 | 1489 | 1928 |
| 203 | g6026147 | 1492 | 1935 |
| 203 | g6036640 | 1492 | 1920 |
| 203 | 6157091H1 | 1492 | 1813 |
| 203 | g6033899 | 1493 | 1928 |
| 203 | g6198865 | 1493 | 1938 |
| 203 | 6219812H2 | 1500 | 1886 |
| 203 | 6192668H1 | 1501 | 1814 |
| 203 | 5768793H1 | 1505 | 1920 |
| 203 | 6191666H1 | 1501 | 1804 |
| 203 | g6473184 | 1502 | 1934 |
| 203 | g6476087 | 1502 | 1928 |
| 203 | 5993364H1 | 1503 | 1813 |
| 203 | g6438645 | 1503 | 1928 |
| 203 | g6076043 | 1504 | 1928 |
| 203 | 6043152H1 | 1519 | 1787 |
| 203 | g6472040 | 1509 | 1937 |
| 203 | 6179110H1 | 1511 | 1806 |
| 203 | g6400409 | 1476 | 1929 |
| 203 | g6036079 | 1476 | 1928 |
| 203 | g6086332 | 1477 | 1934 |
| 203 | g6199417 | 1477 | 1933 |
| 203 | g6044232 | 1478 | 1936 |
| 203 | g6029466 | 1478 | 1935 |
| 203 | g6439518 | 1479 | 1934 |
| 203 | g6462956 | 1479 | 1934 |
| 203 | g6439604 | 1480 | 1937 |
| 203 | g6196631 | 1480 | 1938 |
| 203 | g6439647 | 1479 | 1930 |
| 203 | g6131743 | 1480 | 1935 |
| 203 | g6044247 | 1480 | 1928 |
| 203 | 6034847H1 | 1482 | 1911 |
| 203 | g6117455 | 1481 | 1933 |
| 203 | g6464482 | 1481 | 1928 |
| 203 | g6036141 | 1481 | 1928 |
| 203 | g6047637 | 1481 | 1928 |
| 203 | g6117632 | 1482 | 1930 |
| 203 | g6463078 | 1482 | 1936 |
| 203 | g6035703 | 1480 | 1936 |
| 203 | g6076131 | 1482 | 1932 |
| 203 | g6196780 | 1482 | 1932 |
| 203 | g6031028 | 1483 | 1930 |
| 203 | g6464375 | 1484 | 1928 |
| 203 | g6043231 | 1484 | 1929 |
| 203 | g6117300 | 1485 | 1933 |
| 203 | g6200216 | 1484 | 1938 |
| 203 | g6040103 | 1485 | 1932 |
| 203 | g6399002 | 1485 | 1926 |
| 203 | g6029144 | 1486 | 1934 |
| 203 | g6043655 | 1487 | 1937 |
| 203 | g6196472 | 1489 | 1934 |
| 203 | g6034277 | 1815 | 1920 |
| 203 | g6117025 | 1815 | 1920 |
| 203 | g6199037 | 1815 | 1920 |
| 203 | g6198912 | 1823 | 1928 |
| 203 | g6473327 | 1833 | 1928 |
| 203 | g6463164 | 1841 | 1930 |
| 203 | 5920960H1 | 1844 | 1925 |
| 203 | g6198784 | 1856 | 1926 |
| 203 | 5787770H1 | 1722 | 1981 |
| 203 | 5789227H1 | 1722 | 1927 |
| 203 | g6074506 | 1728 | 1938 |
| 203 | 6166065H1 | 1741 | 1956 |
| 203 | g6029762 | 1746 | 1876 |
| 203 | 5786021H1 | 1754 | 1926 |
| 203 | g6438899 | 1763 | 1928 |
| 203 | 5992585H1 | 1769 | 1920 |
| 203 | 6158235H1 | 1865 | 1940 |
| 203 | 5946071H1 | 1668 | 1917 |
| 203 | 5950118H1 | 1668 | 1905 |
| 203 | 6157445H1 | 1668 | 1850 |
| 203 | 5946973H1 | 1668 | 1920 |
| 203 | 5767935H1 | 1670 | 1920 |
| 203 | 5767973H1 | 1670 | 1912 |
| 203 | 5947508H1 | 1672 | 1934 |
| 203 | 5790266H1 | 1674 | 1920 |
| 203 | g6400408 | 1676 | 1938 |
| 203 | 7338126H1 | 1680 | 1932 |
| 203 | 7350525H1 | 1686 | 1936 |
| 203 | g6398110 | 1686 | 1938 |
| 203 | g6402278 | 1686 | 1938 |
| 203 | 6157246H1 | 1686 | 1827 |
| 203 | g6197883 | 1687 | 1928 |
| 203 | g6046641 | 1691 | 1928 |
| 203 | g6400764 | 1692 | 1928 |
| 203 | g6076220 | 1693 | 1928 |
| 203 | 5794559H1 | 1695 | 1926 |
| 203 | g6033633 | 1695 | 1926 |
| 203 | 5787563H1 | 1695 | 1925 |
| 203 | g6117328 | 1708 | 1937 |
| 203 | g6044253 | 1710 | 1929 |
| 203 | g6398246 | 1721 | 1928 |
| 203 | g6131945 | 1465 | 1929 |
| 203 | 6181714H1 | 1466 | 1806 |
| 203 | g6048081 | 1466 | 1928 |
| 203 | g6047077 | 1466 | 1932 |
| 203 | g6132102 | 1468 | 1929 |
| 203 | g6398647 | 1468 | 1929 |
| 203 | g6400444 | 1468 | 1929 |
| 203 | g6086266 | 1468 | 1928 |
| 203 | g6132321 | 1469 | 1928 |
| 203 | g6132239 | 1470 | 1930 |
| 203 | g6074116 | 1468 | 1928 |
| 203 | 5990574H1 | 1470 | 1804 |
| 203 | g6400620 | 1470 | 1930 |
| 203 | g6074889 | 1470 | 1928 |
| 203 | g6473566 | 1469 | 1928 |
| 203 | g6400824 | 1472 | 1936 |
| 203 | g6132812 | 1472 | 1930 |
| 203 | g6400805 | 1472 | 1938 |
| 203 | g6400717 | 1472 | 1928 |
| 203 | g6030907 | 1473 | 1928 |
| 203 | g6047234 | 1474 | 1930 |
| 203 | 7180844H1 | 1459 | 1928 |
| 203 | g6035938 | 1464 | 1916 |
| 203 | g6439666 | 1475 | 1928 |
| 203 | g6036991 | 1475 | 1928 |
| 203 | g6047968 | 1475 | 1930 |
| 203 | g6036801 | 1476 | 1937 |
| 203 | g6196212 | 1600 | 1928 |
| 203 | 6177877H1 | 1603 | 1888 |
| 203 | g6039199 | 1605 | 1928 |
| 203 | g6438679 | 1604 | 1920 |
| 203 | g6400557 | 1611 | 1928 |
| 203 | 5817262H1 | 1615 | 1904 |
| 203 | 5817362H1 | 1615 | 1906 |
| 203 | 5820474H1 | 1615 | 1915 |
| 203 | 6102129H1 | 1619 | 1928 |
| 203 | g6074067 | 1653 | 1932 |
| 203 | g6132060 | 1656 | 1935 |
| 203 | 3775108F9 | 1656 | 1912 |
| 203 | g6198405 | 1665 | 1929 |
| 203 | 6178153H1 | 1665 | 1928 |
| 203 | g6045038 | 1666 | 1911 |
| 203 | 5770026H1 | 1668 | 1946 |
| 203 | 5792284H1 | 1668 | 1920 |
| 203 | g6028253 | 1588 | 1930 |
| 203 | g6475978 | 1593 | 1932 |
| 203 | 6175948H1 | 1593 | 1891 |
| 203 | g6132639 | 1594 | 1928 |
| 203 | g6463340 | 1597 | 1928 |
| 203 | g6046756 | 1596 | 1928 |
| 204 | 6844571H1 | 664 | 1221 |
| 204 | 1299074T6 | 715 | 1264 |
| 204 | 1751671T6 | 860 | 1258 |
| 204 | 6941680H1 | 769 | 1245 |
| 204 | 1426502T6 | 979 | 1248 |
| 204 | 2825904H1 | 984 | 1241 |
| 204 | 6825511H1 | 594 | 1115 |
| 204 | 6827717H1 | 676 | 1155 |
| 204 | 6828967J1 | 813 | 1145 |
| 204 | 6825030H1 | 823 | 1116 |
| 204 | 6825030J1 | 822 | 1115 |
| 204 | 1470306H1 | 921 | 1114 |
| 204 | 6827717J1 | 434 | 1076 |
| 204 | 1945447H1 | 808 | 1051 |
| 204 | 5870352H1 | 729 | 1007 |
| 204 | 6826536J1 | 1157 | 1756 |
| 204 | 6822128J1 | 829 | 984 |
| 204 | 6826536H1 | 1143 | 1742 |
| 204 | g3056033 | 1065 | 1305 |
| 204 | g5837846 | 816 | 1289 |
| 204 | g2986193 | 1027 | 1297 |
| 204 | 6831310H1 | 845 | 1278 |
| 204 | 1631532H1 | 1227 | 1297 |
| 204 | g3150672 | 914 | 1287 |
| 204 | g2236974 | 899 | 1287 |
| 204 | g4889379 | 1021 | 1302 |
| 204 | 2255054R6 | 393 | 777 |
| 204 | 6498130H1 | 312 | 777 |
| 204 | 2255054H1 | 549 | 777 |
| 204 | 6826447H1 | 357 | 752 |
| 204 | 6826447J1 | 357 | 752 |
| 204 | 1579339H1 | 502 | 713 |
| 204 | 4443559H1 | 474 | 613 |
| 204 | 2042892H1 | 315 | 568 |
| 204 | 3358238H1 | 329 | 566 |
| 204 | 4750087H1 | 185 | 453 |
| 204 | 6824269H1 | 386 | 880 |
| 204 | 6824349H1 | 350 | 851 |
| 204 | 2184748H1 | 179 | 463 |
| 204 | 6756704J1 | 235 | 808 |
| 204 | 2184748F6 | 1 | 463 |
| 204 | 6756496H1 | 234 | 432 |
| 204 | 6756704H1 | 255 | 432 |
| 204 | 1299074H1 | 161 | 401 |
| 204 | 6824091H1 | 156 | 422 |
| 204 | 3698810H1 | 13 | 304 |
| 204 | 1751671H1 | 621 | 843 |
| 204 | g1616031 | 448 | 783 |
| 204 | 6824349J1 | 147 | 796 |
| 204 | 4516155H1 | 541 | 775 |
| 204 | 4672305H1 | 94 | 298 |
| 204 | 6829925H1 | 1303 | 1619 |
| 204 | 1426502H1 | 1438 | 1679 |
| 204 | 6829925J1 | 1303 | 1618 |
| 204 | 6824269J1 | 875 | 1484 |
| 204 | 6824288J1 | 805 | 1385 |
| 204 | 6825511J1 | 786 | 1392 |
| 204 | g5856181 | 886 | 1309 |
| 204 | g824615 | 1112 | 1275 |
| 204 | 6802929J1 | 833 | 1289 |
| 204 | g5127144 | 929 | 1299 |
| 204 | g2344128 | 1075 | 1315 |
| 204 | 6458046H1 | 929 | 1285 |
| 204 | g2397910 | 1064 | 1302 |
| 204 | g2278427 | 1157 | 1301 |
| 204 | g3058045 | 823 | 1284 |
| 204 | 6820991H1 | 747 | 1284 |
| 204 | 2255054T6 | 705 | 1267 |
| 204 | 6822128H1 | 514 | 951 |
| 204 | 6802929H1 | 535 | 920 |
| 204 | 6825384H1 | 424 | 928 |
| 204 | 6824288H1 | 498 | 991 |
| 204 | 6824091J1 | 335 | 879 |
| 204 | 3512547H1 | 1455 | 1695 |
| 204 | 6804257H1 | 1157 | 1691 |
| 204 | 3700061H1 | 1404 | 1683 |
| 204 | 1426502F6 | 1335 | 1679 |
| 204 | 755545H1 | 1439 | 1679 |
| 204 | 6498381H1 | 1158 | 1726 |
| 205 | 1858538T6 | 1354 | 1583 |
| 205 | 1810993T6 | 1360 | 1586 |
| 205 | 1810792H1 | 1281 | 1506 |
| 205 | 2625825H1 | 1367 | 1595 |
| 205 | 2228636T6 | 1368 | 1586 |
| 205 | 1724417T6 | 1386 | 1586 |
| 205 | 2689550T6 | 1333 | 1586 |
| 205 | 2689550F6 | 1 | 511 |
| 205 | 2689550H1 | 1 | 265 |
| 205 | 6476804H1 | 3 | 563 |
| 205 | 1545750H1 | 17 | 163 |
| 205 | g2244465 | 68 | 341 |
| 205 | 1494653H1 | 351 | 402 |
| 205 | 1858538H1 | 400 | 682 |
| 205 | 2218749F6 | 474 | 916 |
| 205 | 2228636F6 | 474 | 908 |
| 205 | 2228636H1 | 474 | 714 |
| 205 | 1724288H1 | 642 | 852 |
| 205 | 1724417H1 | 642 | 852 |
| 205 | 1724417F6 | 642 | 998 |
| 205 | 1724064H1 | 642 | 841 |
| 205 | 1724064F6 | 642 | 1096 |
| 205 | 6549336H1 | 1002 | 1419 |
| 205 | 4822845H1 | 1024 | 1261 |
| 205 | 1810993F6 | 1281 | 1558 |
| 205 | 1810993H1 | 1281 | 1518 |
| 206 | 6045641H1 | 1 | 364 |
| 206 | 6045641J1 | 1 | 364 |
| 206 | 3179825H1 | 120 | 424 |
| 206 | 3179825F6 | 120 | 672 |
| 206 | 4758644H1 | 141 | 409 |
| 206 | 2966833H1 | 222 | 484 |
| 206 | 5372448H1 | 327 | 481 |
| 206 | 2686267H1 | 359 | 619 |
| 206 | 3722865H1 | 425 | 558 |
| 206 | 5036810H1 | 463 | 731 |
| 206 | 4164428H1 | 519 | 787 |
| 206 | 3799675H1 | 563 | 731 |
| 206 | 4708839H1 | 568 | 851 |
| 206 | 2952970H1 | 602 | 865 |
| 206 | 3561988H1 | 633 | 928 |
| 206 | 856538H1 | 642 | 846 |
| 206 | 2487001F6 | 672 | 1061 |
| 206 | 2487001H1 | 672 | 829 |
| 206 | 4980831H1 | 862 | 1147 |
| 206 | 2620607H1 | 875 | 1129 |
| 206 | 3873170H1 | 876 | 1028 |
| 206 | 4575663H1 | 912 | 1190 |
| 206 | 6155913H1 | 953 | 1276 |
| 206 | 4543658H1 | 1005 | 1249 |
| 206 | 5064775H1 | 1104 | 1345 |
| 206 | g1014062 | 1144 | 1418 |
| 206 | 6249620H1 | 1173 | 1668 |
| 206 | 2881132F6 | 1223 | 1489 |
| 206 | 2881132H1 | 1223 | 1496 |
| 206 | g1949025 | 1228 | 1546 |
| 206 | 4552262H1 | 1238 | 1500 |
| 206 | 950155H1 | 1278 | 1523 |
| 206 | 1705026H1 | 1316 | 1527 |
| 206 | 4398180H1 | 1331 | 1571 |
| 206 | 3179825T6 | 1418 | 1909 |
| 206 | 2487001T6 | 1441 | 2006 |
| 206 | 6097383H1 | 1482 | 1798 |
| 206 | g2251371 | 1596 | 2047 |
| 206 | g4072810 | 1631 | 2050 |
| 206 | g3889774 | 1700 | 2050 |
| 206 | g4087488 | 1749 | 2056 |
| 206 | g1014063 | 1798 | 2030 |
| 206 | 2881132T6 | 1809 | 2005 |
| 206 | g1195443 | 1885 | 2047 |
| 206 | g2155148 | 1942 | 2053 |
| 207 | 6803514H1 | 503 | 984 |
| 207 | 6803514J1 | 242 | 823 |
| 207 | 6286909H2 | 316 | 799 |
| 207 | g4568721 | 260 | 685 |
| 207 | g3742689 | 209 | 684 |
| 207 | g5365250 | 586 | 679 |
| 207 | g5676607 | 451 | 679 |
| 207 | g4111591 | 263 | 679 |
| 207 | g2539496 | 299 | 678 |
| 207 | g5769156 | 224 | 677 |
| 207 | g5232991 | 277 | 675 |
| 207 | g3090059 | 202 | 676 |
| 207 | g3751723 | 261 | 676 |
| 207 | g4299176 | 248 | 676 |
| 207 | g5630519 | 273 | 675 |
| 207 | 5743674T7 | 354 | 545 |
| 207 | 5743674H1 | 1 | 297 |
| 207 | 4127943H1 | 1 | 246 |
| 207 | 5743674R7 | 1 | 183 |
| 208 | 1952854T6 | 1 | 565 |
| 209 | 2917244T6 | 1 | 567 |
| 209 | 2917244H1 | 1 | 156 |
| 209 | 2917244F6 | 1 | 567 |
| 210 | 5507875H1 | 1 | 219 |
| 210 | 5507875F6 | 1 | 393 |
| 210 | 3510289H1 | 1 | 276 |
| 210 | 6985373H1 | 20 | 382 |
| 210 | 6985355H1 | 20 | 540 |
| 210 | 4715879H1 | 20 | 238 |
| 210 | 645341H1 | 21 | 220 |
| 210 | 645341R6 | 21 | 432 |
| 210 | 4551228H1 | 20 | 212 |
| 210 | 3182009H1 | 24 | 326 |
| 210 | 3182022H1 | 24 | 321 |
| 210 | 5156573H1 | 27 | 272 |
| 210 | 4800844H1 | 28 | 277 |
| 210 | 5864355H1 | 31 | 324 |
| 210 | 6219781H1 | 32 | 251 |
| 210 | 3375196H1 | 35 | 280 |
| 210 | 6306192H1 | 38 | 512 |
| 210 | g575091 | 38 | 316 |
| 210 | 2924014F6 | 38 | 385 |
| 210 | 2924014H1 | 38 | 247 |
| 210 | 2825826H1 | 56 | 355 |
| 210 | 3695750H1 | 62 | 182 |
| 210 | 941644R6 | 66 | 275 |
| 210 | 5310424H1 | 66 | 282 |
| 210 | 3580982H1 | 36 | 340 |
| 210 | 941644R1 | 66 | 365 |
| 210 | 5967756H1 | 70 | 570 |
| 210 | g831059 | 85 | 445 |
| 210 | 5727850H1 | 131 | 666 |
| 210 | 2053435H1 | 218 | 446 |
| 210 | g4682505 | 234 | 685 |
| 210 | 5507875R6 | 308 | 769 |
| 210 | 6874493H1 | 354 | 971 |
| 210 | g5109730 | 543 | 998 |
| 210 | g1391905 | 560 | 858 |
| 211 | 6409981H1 | 1 | 486 |

TABLE 5

| SEQ ID NO: | Template ID | Tissue Distribution |
|---|---|---|
| 1 | LG:1040582.1:2000FEB18 | Liver - 41%, Pancreas - 34%, Cardiovascular System - |
| | | 14% |
| 2 | LG:453570.1:2000FEB18 | Nervous System - 100% |
| 3 | LG:408751.3:2000FEB18 | Sense Organs - 63%, Nervous System - 22% |
| 4 | LI:090574.1:2000FEB01 | Nervous System - 46%, Unclassified/Mixed - 36% |
| 5 | LI:229932.2:2000FEB01 | Musculoskeletal System - 80% |
| 6 | LI:332176.1:2000FEB01 | Urinary Tract - 95% |
| 7 | LI:403248.2:2000FEB01 | Respiratory System - 60%, Hemic and Immune |
| | | System - 40% |
| 8 | LG:220992.1:2000MAY19 | Embryonic Structures - 17%, Male Genitalia - 12% |
| 9 | LG:1094571.1:2000MAY19 | Liver - 19%, Embryonic Structures - 16%, |
| | | Cardiovascular System - 14% |
| 10 | LI:350754.4:2000MAY01 | Skin - 47%, Stomatognathic System - 27%, Sense |
| | | Organs - 14% |
| 11 | LI:255828.29:2000MAY01 | Musculoskeletal System - 100% |
| 12 | LI:1190263.1:2000MAY01 | Urinary Tract - 80%, Urinary Tract - 15% |
| 13 | LG:270916.2:2000FEB18 | Female Genitalia - 100% |
| 14 | LG:999414.3:2000FEB18 | Embryonic Structures - 30%, Urinary Tract - 13%, |
| | | Digestive System - 11%, Musculoskeletal System - |
| | | 11% |
| 15 | LG:429446.1:2000FEB18 | Urinary Tract - 80%, Hemic and Immune System - |
| | | 20% |
| 16 | LI:057229.1:2000FEB01 | Male Genitalia - 71%, Hemic and Immune System - |
| | | 29% |
| 17 | LI:351965.1:2000FEB01 | Unclassified/Mixed - 53%, Male Genitalia - 12% |
| 18 | LG:068682.1:2000FEB18 | Unclassified/Mixed - 49%, Male Genitalia - 27% |
| 19 | LG:242665.1:2000FEB18 | Germ Cells - 47%, Female Genitalia - 13%, Male |
| | | Genitalia - 12% |
| 20 | LG:241743.1:2000FEB18 | Liver - 27%, Urinary Tract - 27%, Respiratory System - |
| | | 14% |
| 21 | LI:034212.1:2000FEB01 | Digestive System - 24%, Musculoskeletal System - |
| | | 22%, Nervous System - 11% |
| 22 | LG:344886.1:2000MAY19 | Germ Cells - 24%, Nervous System - 12% |
| 23 | LG:228930.1:2000MAY19 | Embryonic Structures - 43%, Nervous System - 29%, |
| | | Respiratory System - 14%, Hemic and Immune |
| | | System - 14% |
| 24 | LG:338927.1:2000MAY19 | Digestive System - 23%, Unclassified/Mixed - 21%, |
| | | Embryonic Structures - 19%, Hemic and Immune |
| | | System - 19% |
| 25 | LG:898771.1:2000MAY19 | Pancreas - 13%, Embryonic Structures - 11%, |
| | | Female Genitalia - 10%, Urinary Tract - 10%, Hemic |
| | | and Immune System - 10%, Cardiovascular System - |
| | | 10% |
| 26 | LI:257664.67:2000MAY01 | Hemic and Immune System - 100% |
| 27 | LI:001496.2:2000MAY01 | Endocrine System - 27%, Female Genitalia - 25%, |
| | | Embryonic Structures - 25% |
| 28 | LI:1085273.2:2000MAY01 | Digestive System - 29%, Skin - 24%, Endocrine |
| | | System - 16% |
| 29 | LI:333138.2:2000MAY01 | Exocrine Glands - 61%, Nervous System - 13%, |
| | | Nervous System - 11% |
| 30 | LI:338927.1:2000MAY01 | Embryonic Structures - 51%, Digestive System - 17% |
| 31 | LG:335558.1:2000FEB18 | Endocrine System - 45%, Nervous System - 18%, |
| | | Exocrine Glands - 11% |
| 32 | LG:998283.7:2000FEB18 | Sense Organs - 33%, Germ Cells - 18% |
| 33 | LI:402739.1:2000FEB01 | Unclassified/Mixed - 78%, Mate Genitalia - 11%, |
| | | Hemic and Immune System - 11% |
| 34 | LI:175223.1:2000FEB01 | Embryonic Structures - 99% |
| 35 | LG:981076.2:2000MAY19 | Endocrine System - 28%, Nervous System - 22%, |
| | | Respiratory System - 17%, Female Genitalia - 17%, |
| | | Hemic and Immune System - 17% |
| 36 | LI:1008973.1:2000MAY01 | Nervous System - 57%, Digestive System - 41% |
| 37 | LI:1190250.1:2000MAY01 | Female Genitalia - 48%, Respiratory System - 25% |
| 38 | LG:021371.3:2000FEB18 | Liver - 23%, Endocrine System - 17%, Hemic and |
| | | Immune System - 17% |
| 39 | LG:475404.1:2000FEB18 | Skin - 82% |
| 40 | LG:979406.2:2000FEB18 | Liver - 46%, Connective Tissue - 31%, Nervous |
| | | System - 15% |
| 41 | LG:410726.1:2000FEB18 | Embryonic Structures - 52%, Endocrine System - |
| | | 26% |
| 42 | LG:200005.1:2000FEB18 | Unclassified/Mixed - 26%, Cardiovascular System - |
| | | 14%, Female Genitalia - 13% |
| 43 | LG:1076828.1:2000FEB18 | Unclassified/Mixed - 69%, Urinary Tract - 25% |
| 44 | LG:1076931.1:2000FEB18 | Unclassified/Mixed - 63%, Musculoskeletal System - |
| | | 20%, Urinary Tract - 11% |
| 45 | LG:1078121.1:2000FEB18 | Female Genitalia - 75%, Nervous System - 25% |
| 46 | LG:1079203.1:2000FEB18 | Female Genitalia - 42%, Cardiovascular System - |
| | | 33%, Hemic and Immune System - 17% |
| 47 | LG:1082586.1:2000FEB18 | Respiratory System - 100% |
| 48 | LG:1082774.1:2000FEB18 | Respiratory System - 50%, Female Genitalia - 50% |
| 49 | LG:1082775.1:2000FEB18 | Female Genitalia - 75%, Nervous System - 25% |
| 50 | LG:1083120.1:2000FEB18 | Nervous System - 100% |
| 51 | LG:1087707.1:2000FEB18 | Stomatognathic System - 98% |
| 52 | LG:1090915.1:2000FEB18 | Embryonic Structures - 44%, Connective tissue - |
| | | 19% |
| 53 | LG:1094230.1:2000FEB18 | Female Genitalia - 100% |
| 54 | LG:474848.3:2000FEB18 | Connective Tissue - 44%, Exocrine Glands - 44%, |
| | | Hemic and Immune System - 11% |
| 55 | LI:251656.1:2000FEB01 | Nervous System - 38%, Digestive System - 38%, Male |
| | | Genitalia - 25% |
| 56 | LI:021371.1:2000FEB01 | Hemic and Immune System - 69%, Endocrine |
| | | System - 14% |
| 57 | LI:133095.1:2000FEB01 | Respiratory System - 67%, Nervous System - 13% |
| 58 | LI:236654.2:2000FEB01 | Unclassified/Mixed - 30%, Respiratory System - 19%, |
| | | Nervous System - 13%, Digestive System - 13% |
| 59 | LI:200009.1:2000FEB01 | Unclassified/Mixed - 37%, Urinary Tract - 16%, |
| | | Cardiovascular System - 15% |
| 60 | LI:758502.1:2000FEB01 | Unclassified/Mixed - 78%, Musculoskeletal System - |
| | | 22% |
| 61 | LI:344772.1:2000FEB01 | Nervous System - 56%, Skin - 27%, Connective Tissue - |
| | | 13% |
| 62 | LI:789445.1:2000FEB01 | Endocrine System - 100% |
| 63 | LI:789657.1:2000FEB01 | Urinary Tract - 31%, Female Genitalia - 19%, |
| | | Digestive System - 19%, Hemic and Immune System - |
| | | 19% |
| 64 | LI:789808.1:2000FEB01 | Exocrine Glands - 44%, Female Genitalia - 33%, |
| | | Nervous System - 22% |
| 65 | LI:792919.1:2000FEB01 | Respiratory System - 100% |
| 66 | LI:793949.1:2000FEB01 | Female Genitalia - 42%, Endocrine System - 19%, |
| | | Exocrine Glands - 13% |
| 67 | LI:794389.1:2000FEB01 | Endocrine System - 100% |
| 68 | LI:796010.1:2000FEB01 | Exocrine Glands - 100% |
| 69 | LI:796324.1:2000FEB01 | Female Genitalia - 100% |
| 70 | LI:796373.1:2000FEB01 | Respiratory System - 100% |
| 71 | LI:796415.1:2000FEB01 | Nervous System - 100% |
| 72 | LI:798636.1:2000FEB01 | Hemic and Immune System - 100% |
| 73 | LI:800045.1:2000FEB01 | Female Genitalia - 60%, Male Genitalia - 40% |
| 74 | LI:800680.1:2000FEB01 | Cardiovascular System - 100% |
| 75 | LI:800894.1:2000FEB01 | Respiratory System - 50%, Digestive System - 50% |
| 76 | LI:801015.1:2000FEB01 | Male Genitalia - 100% |
| 77 | LI:801236.1:2000FEB01 | Endocrine System - 100% |
| 78 | LI:803335.1:2000FEB01 | Connective Tissue - 100% |
| 79 | LI:803998.1:2000FEB01 | Nervous System - 38%, Digestive System - 38%, Male |
| | | Genitalia - 25% |
| 80 | LI:478757.1:2000FEB01 | Digestive System - 100% |
| 81 | LI:808532.1:2000FEB01 | Hemic and Immune System - 100% |
| 82 | LI:443073.1:2000FEB01 | Digestive System - 100% |
| 83 | LI:479671.1:2000FEB01 | Exocrine Glands - 80%, Hemic and Immune System - |
| | | 20% |
| 84 | LI:810078.1:2000FEB01 | Digestive System - 100% |
| 85 | LI:810224.1:2000FEB01 | Digestive System - 100% |
| 86 | LI:817052.2:2000FEB01 | Nervous System - 24%, Unclassified/Mixed - 18%, |
| | | Exocrine Glands - 14% |
| 87 | LG:892274.1:2000MAY19 | Embryonic Structures - 63%, Digestive System - 30% |
| 88 | LG:1080959.1:2000MAY19 | Digestive System - 40%, Respiratory System - 30%, |
| | | Hemic and Immune System - 30% |
| 89 | LG:1054900.1:2000MAY19 | Digestive System - 100% |
| 90 | LG:1077357.1:2000MAY19 | Nervous System - 38%, Female Genitalia - 38%, |
| | | Male Genitalia - 25% |
| 91 | LG:1084051.1:2000MAY19 | Pancreas - 31%, Digestive System - 22%, Hemic and |
| | | Immune System - 16% |
| 92 | LG:1076853.1:2000MAY19 | Female Genitalia - 23%, Unclassified/Mixed - 23%, |
| | | Cardiovascular System - 18%, Exocrine Glands - |
| | | 18% |
| 93 | LG:481631.10:2000MAY19 | Female Genitalia - 22%, Nervous System - 17%, |
| | | Exocrine Glands - 17%, Urinary Tract - 17% |
| 94 | LG:1088431.2:2000MAY19 | Exocrine Glands - 67%, Cardiovascular System - |
| | | 33% |
| 95 | LI:401619.10:2000MAY01 | Endocrine System - 18%, Embryonic Structures - |
| | | 16%, Pancreas - 15% |
| 96 | LI:1144007.1:2000MAY01 | Hemic and Immune System - 27%, Female |
| | | Genitalia - 13% |
| 97 | LI:331074.1:2000MAY01 | Endocrine System - 28%, Sense Organs - 22%, |
| | | Connective Tissue - 10% |
| 98 | LI:1170349.1:2000MAY01 | Endocrine System - 91% |
| 99 | LG:335097.1:2000FEB18 | Embryonic Structures - 24%, Musculoskeletal System - |
| | | 19%, Nervous System - 16% |
| 100 | LG:1076451.1:2000FEB18 | Nervous System - 100% |
| 101 | LI:805478.1:2000FEB01 | Skin - 100% |
| 102 | LG:101269.1:2000MAY19 | Endocrine System - 33%, Embryonic Structures - |
| | | 33%, Urinary Tract - 30% |
| 103 | LI:331087.1:2000MAY01 | Liver - 82%, Hemic and Immune System - 13% |
| 104 | LI:410188.1:2000MAY01 | Cardiovascular System - 81%, Cardiovascular |
| | | System - 12% |
| 105 | LI:1188288.1:2000MAY01 | Nervous System - 73% |
| 106 | LI:427997.4:2000MAY01 | Liver - 16%, Male Genitalia - 13%, Embryonic |
| | | Structures - 11% |
| 107 | LG:451682.1:2000FEB18 | Nervous System - 100% |
| 108 | LG:1077283.1:2000FEB18 | Liver - 86%, Hemic and Immune System - 14% |
| 109 | LG:481436.5:2000FEB18 | Embryonic Structures - 41%, Endocrine System - |
| | | 20%, Hemic and Immune System - 13% |
| 110 | LI:793701.1:2000FEB01 | Endocrine System - 43%, Urinary Tract - 36%, |
| | | Respiratory System - 21% |
| 111 | LI:373637.1:2000FEB01 | Germ Cells - 74%, Unclassified/Mixed - 16% |
| 112 | LG:239368.2:2000MAY19 | Digestive System - 43%, Male Genitalia - 24%, |
| | | Endocrine System - 24% |
| 113 | LI:053826.1:2000MAY01 | Germ Cells - 66%, Unclassified/Mixed - 22%, Male |
| | | Genitalia - 12% |
| 114 | LI:449393.1:2000MAY01 | Nervous System - 100% |
| 115 | LI:1071427.96:2000MAY01 | Stomatognathic System - 13% |
| 116 | LI:336338.8:2000MAY01 | Unclassified/Mixed - 55%, Connective Tissue - 26% |
| 117 | LG:345527.1:2000FEB18 | Urinary Tract - 24%, Hemic and Immune System - |
| | | 24%, Respiratory System - 18% |
| 118 | LG:1089383.1:2000FEB18 | Connective Tissue - 73%, Female Genitalia - 27% |
| 119 | LG:1092522.1:2000FEB18 | Female Genitalia - 38%, Exocrine Glands - 31%, |
| | | Male Genitalia - 15%, Hemic and Immune System - |
| | | 15% |
| 120 | LG:1093216.1:2000FEB18 | Urinary Tract - 100% |
| 121 | LI:270318.3:2000FEB01 | Embryonic Structures - 86%, Hemic and Immune |
| | | System - 14% |
| 122 | LI:335671.2:2000FEB01 | Unclassified/Mixed - 34%, Hemic and Immune |
| | | System - 20%, Urinary Tract - 17% |
| 123 | LI:793758.1:2000FEB01 | Nervous System - 62%, Urinary Tract - 38% |
| 124 | LI:803718.1:2000FEB01 | Female Genitalia - 100% |
| 125 | LI:412179.1:2000FEB01 | Endocrine System - 100% |
| 126 | LI:815679.1:2000FEB01 | Digestive System - 75% |
| 127 | LI:481361.3:2000FEB01 | Embryonic Structures - 28%, Skin - 20%, |
| | | Unclassified/Mixed - 16% |
| 128 | LG:247388.1:2000MAY19 | Cardiovascular System - 33%, Endocrine System - |
| | | 21%, Male Genitalia - 21% |
| 129 | LG:255789.10:2000MAY19 | Endocrine System - 56%, Urinary Tract - 44% |
| 130 | LI:787618.1:2000MAY01 | Endocrine System - 22%, Digestive System - 13%, |
| | | Endocrine System - 12% |
| 139 | LG:337818.2:2000FEB18 | Sense Organs - 18%, Nervous System - 11%, |
| | | Digestive System - 34%, Liver - 17%, Female |
| | | Genitalia - 11% |
| 140 | LI:337818.1:2000FEB01 | Digestive System - 27%, Liver - 19%, Female |
| | | Genitalia - 15% |
| 141 | LG:241577.4:2000MAY19 | Pancreas - 48%, Endocrine System - 24%, |
| | | Respiratory System - 14% |
| 142 | LG:344786.4:2000MAY19 | Respiratory System - 67%, Digestive System - 22%, |
| | | Nervous System - 11% |
| 143 | LI:414307.1:2000FEB01 | Endocrine System - 44%, Unclassified/Mixed - 17%, |
| | | Nervous System - 11% |
| 144 | LI:202943.2:2000FEB01 | Embryonic Structures - 100% |
| 145 | LI:246194.2:2000FEB01 | Germ Cells - 75%, Pancreas - 13% |
| 146 | LI:815961.1:2000FEB01 | Digestive System - 99% |
| 147 | LG:120744.1:2000MAY19 | Skin - 33%, Embryonic Structures - 21%, Digestive |
| | | System - 21% |
| 148 | LI:757520.1:2000MAY01 | Musculoskeletal System - 45%, Cardiovascular |
| | | System - 26%, Skin - 24% |
| 149 | LG:160570.1:2000FEB18 | Skin - 84%, Female Genitalia - 16% |
| 150 | LI:350398.3:2000FEB01 | Male Genitalia - 50%, Hemic and Immune System - |
| | | 50% |
| 151 | LI:221285.1:2000FEB01 | Endocrine System - 42%, Nervous System - 21% |
| 153 | LI:329017.1:2000FEB01 | Endocrine System - 62%, Unclassified/Mixed - 24% |
| 154 | LI:401322.1:2000FEB01 | Sense Organs - 44%, Liver - 22%, Skin - 14% |
| 155 | LG:403409.1:2000MAY19 | Respiratory System - 18%, Female Genitalia - 16%, |
| | | Cardiovascular System - 13% |
| 156 | LG:233933.5:2000MAY19 | Digestive System - 100% |
| 157 | LI:290344.1:2000MAY01 | Connective Tissue - 40%, Nervous System - 19%, |
| | | Embryonic Structures - 12% |
| 158 | LI:410742.1:2000MAY01 | Respiratory System - 47%, Skin - 42% |
| 159 | LG:406568.1:2000MAY19 | Stomatognathic System - 57%, Musculoskeletal |
| | | System - 21%, Cardiovascular System - 16% |
| 160 | LI:283762.1:2000MAY01 | Sense Organs - 25% |
| 161 | LI:347687.113:2000MAY01 | Nervous System - 45%, Nervous System - 38% |
| 162 | LI:1146510.1:2000MAY01 | Skin - 94% |
| 163 | LG:451710.1:2000FEB18 | Connective Tissue - 89%, Nervous System - 11% |
| 164 | LG:455771.1:2000FEB18 | Nervous System - 100% |
| 165 | LG:452089.1:2000FEB18 | Nervous System - 100% |
| 166 | LG:246415.1:2000FEB18 | Pancreas - 83%, Nervous System - 17% |
| 167 | LG:414144.10:2000FEB18 | Cardiovascular System - 17%, Connective Tissue - |
| | | 12% |
| 168 | LG:1101445.1:2000FEB18 | Liver - 91% |
| 169 | LG:452134.1:2000FEB18 | Hemic and Immune System - 64%, Male Genitalia - |
| | | 36% |
| 170 | LI:903021.1:2000FEB01 | Male Genitalia - 100% |
| 171 | LI:246422.1:2000FEB01 | Hemic and Immune System - 100% |
| 172 | LG:449404.1:2000MAY19 | Nervous System - 100% |
| 173 | LG:449413.1:2000MAY19 | Nervous System - 100% |
| 174 | LG:450105.1:2000MAY19 | Nervous System - 100% |
| 175 | LG:460809.1:2000MAY19 | Exocrine Glands - 100% |
| 176 | LG:481781.1:2000MAY19 | Nervous System - 100% |
| 177 | LG:1101153.1:2000MAY19 | Nervous System - 100% |
| 178 | LI:257695.20:2000MAY01 | Exocrine Glands - 28%, Endocrine System - 19%, |
| | | Nervous System - 16%, Digestive System - 16% |
| 179 | LI:455771.1:2000MAY01 | Nervous System - 100% |
| 180 | LI:274551.1:2000MAY01 | Nervous System - 60%, Hemic and Immune System - |
| | | 40% |
| 181 | LI:035973.1:2000MAY01 | Embryonic Structures - 58%, Digestive System - 26%, |
| | | Nervous System - 16% |
| 182 | LG:978427.5:2000FEB18 | Nervous System - 100% |
| 183 | LG:247781.2:2000FEB18 | Nervous System - 11% |
| 184 | LI:034583.1:2000FEB01 | Nervous System - 35%, Endocrine System - 35% |
| 185 | LI:333307.2:2000FEB01 | Cardiovascular System - 28%, Urinary Tract - 27%, |
| | | Musculoskeletal System - 17% |
| 186 | LI:814710.2:2000FEB01 | Respiratory System - 100% |
| 187 | LG:414732.1:2000MAY19 | Endocrine System - 82%, Nervous System - 18% |
| 188 | LG:413910.6:2000MAY19 | Connective Tissue - 55%, Nervous System - 15%, |
| | | Embryonic Structures - 13% |
| 189 | LI:414732.2:2000MAY01 | Endocrine System - 80%, Nervous System - 20% |
| 190 | LI:900264.2:2000MAY01 | Urinary Tract - 15%, Male Genitalia - 12% |
| 191 | LI:335593.1:2000MAY01 | Urinary Tract - 46%, Endocrine System - 17%, Germ |
| | | Cells - 14% |
| 192 | LI:1189543.1:2000MAY01 | Stomatognathic System - 35%, Digestive System - |
| | | 14% |
| 193 | LG:455450.1:2000FEB18 | Nervous System - 100% |
| 194 | LG:1040978.1:2000FEB18 | Nervous System - 100% |
| 195 | LG:446649.1:2000FEB18 | Liver - 80%, Hemic and Immune System - 13% |
| 196 | LG:132147.3:2000FEB18 | Unclassified/Mixed - 17%, Sense Organs - 16%, |
| | | Embryonic Structures - 10% |
| 197 | LI:036034.1:2000FEB01 | Nervous System - 80% |
| 198 | LG:162161.1:2000MAY19 | Unclassified/Mixed - 53%, Cardiovascular System - |
| | | 21%, Nervous System - 16% |
| 199 | LG:407214.10:2000MAY19 | Unclassified/Mixed - 40%, Respiratory System - 24%, |
| | | Cardiovascular System - 16% |
| 200 | LG:204626.1:2000MAY19 | Digestive System - 41%, Exocrine Glands - 24%, |
| | | Female Genitalia - 18% |
| 201 | LI:007401.1:2000MAY01 | Unclassified/Mixed - 31%, Nervous System - 25%, |
| | | Urinary Tract - 11% |
| 202 | LI:476342.1:2000MAY01 | Connective Tissue - 77%, Nervous System - 23% |
| 203 | LI:1072759.1:2000MAY01 | Hemic and Immune System - 27%, Musculoskeletal |
| | | System - 19%, Endocrine System - 11% |
| 204 | LG:998857.1:2000FEB18 | Digestive System - 58%, Pancreas - 12% |
| 205 | LG:482261.1:2000FEB18 | Male Genitalia - 85%, Respiratory System - 15% |
| 206 | LG:480328.1:2000FEB18 | Skin - 20%, Germ Cells - 18%, Female Genitalia - |
| | | 10% |
| 207 | LG:311197.1:2000MAY19 | Germ Cells - 44%, Digestive System - 15%, Male |
| | | Genitalia - 11% |
| 208 | LG:1054883.1:2000MAY19 | Endocrine System - 100% |
| 209 | LG:399395.1:2000MAY19 | Hemic and Immune System - 100% |
| 210 | LG:380497.2:2000MAY19 | Germ Cells - 23%, Exocrine Glands - 14%, |
| | | Connective Tissue - 13% |
| 211 | LI:272913.22:2000MAY01 | Female Genitalia - 100% |

TABLE 6

| SEQ | | | | | | | |
|---|---|---|---|---|---|---|---|
| ID | | | | | | Probability | |
| NO: | Frame | Length | Start | Stop | GI Number | Score | Annotation |
| 212 | 3 | 115 | 198 | 542 | g399660 | 3.00E−51 | aldehyde reductase [Rattus norvegicus] |
| 212 | 3 | 115 | 198 | 542 | g7677318 | 7.00E−51 | aldehyde reductase [Mus musculus] |
| 212 | 3 | 115 | 198 | 542 | g6013149 | 2.00E−48 | aldehyde reductase [Homo sapiens] |
| 213 | 3 | 161 | 3 | 485 | g2909424 | 2.00E−60 | Glyoxalase I [Cicer arietinum] |
| 213 | 3 | 161 | 3 | 485 | g2113825 | 2.00E−58 | Glyoxalase I [Brassica juncea] |
| 213 | 3 | 161 | 3 | 485 | g1177314 | 4.00E−57 | glyoxalase-I [Lycopersicon esculentum] |
| 214 | 2 | 332 | 2 | 997 | g8671168 | 0 | hypothetical protein [Homo sapiens] |
| 214 | 2 | 332 | 2 | 997 | g8886025 | 0 | collapsin response mediator protein-5 [Homo sapiens] |
| 214 | 2 | 332 | 2 | 997 | g8671360 | 1.00E−179 | Ulip-like protein [Rattus norvegicus] |
| 215 | 3 | 274 | 12 | 833 | g29600 | 2.00E−86 | carbonic anhydrase I (AA 1-261) [Homo sapiens] |
| 215 | 3 | 274 | 12 | 833 | g179793 | 2.00E−86 | carbonic anhydrase I (EC 4.2.1.1) [Homo sapiens] |
| 215 | 3 | 274 | 12 | 833 | g29587 | 4.00E−84 | carbonic anhydrase II (AA 1-260) [Homo sapiens] |
| 216 | 1 | 182 | 742 | 1287 | g10438188 | 1.00E−102 | unnamed protein product [Homo sapiens] |
| 216 | 1 | 182 | 742 | 1287 | g9949721 | 3.00E−49 | probable acetyl-coa synthetase [Pseudomonas aeruginosa] |
| 216 | 1 | 182 | 742 | 1287 | g9655831 | 7.00E−46 | prpE protein [Vibrio cholerae] |
| 217 | 2 | 359 | 2 | 1078 | g2104689 | 1.00E−111 | alpha glucosidase II, alpha subunit [Mus musculus] |
| 217 | 2 | 359 | 2 | 1078 | g7672977 | 1.00E−111 | glucosidase II alpha subunit [Homo sapiens] |
| 217 | 2 | 359 | 2 | 1078 | g577295 | 1.00E−110 | The ha1225 gene product is related to human alpha-glucosidase. [Homo |
| | | | | | | | sapiens] |
| 218 | 2 | 110 | 161 | 490 | g9653274 | 1.00E−26 | ornithine decarboxylase-2 [Xenopus laevis] |
| 218 | 2 | 110 | 161 | 490 | g200124 | 5.00E−18 | ornithine decarboxylase [Mus pahari] |
| 218 | 2 | 110 | 161 | 490 | g53518 | 1.00E−17 | ornithine decarboxylase [Mus musculus] |
| 219 | 3 | 549 | 36 | 1682 | g10435462 | 0 | unnamed protein product [Homo sapiens] |
| 219 | 3 | 549 | 36 | 1682 | g7023375 | 0 | unnamed protein product [Homo sapiens] |
| 219 | 3 | 549 | 36 | 1682 | g10433608 | 1.00E−164 | unnamed protein product [Homo sapiens] |
| 220 | 1 | 264 | 1 | 792 | g7023634 | 3.00E−92 | unnamed protein product [Homo sapiens] |
| 220 | 1 | 264 | 1 | 792 | g3213202 | 3.00E−49 | similarto C. elegans R10H10.6 and S. cerevisiae YD8419.03c [Drosophila |
| | | | | | | | melanogaster] |
| 220 | 1 | 264 | 1 | 792 | g7298960 | 3.00E−49 | CG2846 gene product [Drosophila melanogaster] |
| 221 | 3 | 701 | 33 | 2135 | g307504 | 0 | transglutaminase E3 [Homo sapiens] |
| 221 | 3 | 701 | 33 | 2135 | g4467804 | 0 | TGM3 (PROTEIN-GLUTAMINE GLUTAMYLTRANSFERASE E3 |
| | | | | | | | PRECURSOR (EC 2.3.2.13) (TGASE E3) (TRANSGLUTAMINASE 3).) [Homo |
| | | | | | | | sapiens] |
| 221 | 3 | 701 | 33 | 2135 | g309521 | 0 | transglutaminase E3 [Mus musculus] |
| 222 | 2 | 150 | 2 | 451 | g35505 | 7.00E−65 | pyruvate kinase [Homo sapiens] |
| 222 | 2 | 150 | 2 | 451 | g189998 | 7.00E−65 | M2-type pyruvate kinase [Homo sapiens] |
| 222 | 2 | 150 | 2 | 451 | g2623945 | 3.00E−64 | pyruvate kinase; ATP: pyruvate 2-o-phosphotransferase [Oryctolagus |
| | | | | | | | cuniculus] |
| 223 | 2 | 234 | 866 | 1567 | g2576305 | 1.00E−128 | arylsulphatase [Homo sapiens] |
| 223 | 2 | 234 | 866 | 1567 | g791002 | 3.00E−82 | ARSD [Homo sapiens] |
| 223 | 2 | 234 | 866 | 1567 | g791004 | 4.00E−75 | ARSE [Homo sapiens] |
| 224 | 2 | 86 | 2 | 259 | | | |
| 225 | 2 | 173 | 1049 | 1567 | g4092820 | 8.00E−62 | BC319430_7 [Homo sapiens] |
| 225 | 2 | 173 | 1049 | 1567 | g2792016 | 2.00E−54 | olfactory receptor [Homo sapiens] |
| 225 | 2 | 173 | 1049 | 1567 | g4092819 | 2.00E−54 | BC319430_5 [Homo sapiens] |
| 226 | 2 | 68 | 86 | 289 | g8272468 | 4.00E−15 | envelope protein [Homo sapiens] |
| 226 | 2 | 68 | 86 | 289 | g4773880 | 4.00E−15 | envelope protein precursor [Homo sapiens] |
| 226 | 2 | 68 | 86 | 289 | g4262296 | 4.00E−15 | envelope protein [Homo sapiens] |
| 227 | 1 | 70 | 79 | 288 | g11231093 | 1.00E−11 | hypothetical protein [Macaca fascicularis] |
| 227 | 1 | 70 | 79 | 288 | g10435559 | 3.00E−10 | unnamed protein product [Homo sapiens] |
| 227 | 1 | 70 | 79 | 288 | g7020625 | 2.00E−09 | unnamed protein product [Homo sapiens] |
| 228 | 2 | 117 | 836 | 1186 | g5726235 | 3.00E−18 | unknown protein U5/2 [multiple sclerosis associated retrovirus element] |
| 229 | 2 | 294 | 2 | 883 | g404634 | 1.00E−59 | serine/threonine kinase [Mus musculus] |
| 229 | 2 | 294 | 2 | 883 | g2738898 | 3.00E−59 | protein kinase [Mus musculus] |
| 229 | 2 | 294 | 2 | 883 | g8101585 | 2.00E−54 | testis specific serine kinase-3 [Mus musculus] |
| 230 | 1 | 326 | 1 | 978 | g2117166 | 1.00E−160 | Ras like GTPase [Homo sapiens] |
| 230 | 1 | 326 | 1 | 978 | g466271 | 1.00E−140 | Rar protein [Homo sapiens] |
| 230 | 1 | 326 | 1 | 978 | g3036779 | 1.00E−102 | match: multiple proteins; RAR (RAS like GTPASE) like [Homo sapiens] |
| 231 | 1 | 182 | 40 | 585 | g5763838 | 1.00E−66 | dJ593C16.1 (ras GTPase activating protein) [Homo sapiens] |
| 231 | 1 | 182 | 40 | 585 | g4417207 | 1.00E−66 | synGAP-d [Rattus norvegicus] |
| 231 | 1 | 182 | 40 | 585 | g4105589 | 1.00E−66 | nGAP [Homo sapiens] |
| 232 | 1 | 358 | 58 | 1131 | g1469876 | 1.00E−103 | The KIAA0147 gene product is related to adenylyl cyclase. [Homo sapiens] |
| 232 | 1 | 358 | 58 | 1131 | g6850952 | 1.00E−86 | vartul-2 protein [Drosophila melanogaster] |
| 232 | 1 | 358 | 58 | 1131 | g6782322 | 1.00E−86 | Vartul-1 protein [Drosophila melanogaster] |
| 233 | 1 | 194 | 370 | 951 | g7008402 | 1.00E−107 | kappa B-ras 1 [Homo sapiens] |
| 233 | 1 | 194 | 370 | 951 | g7239257 | 1.00E−103 | kappaB-Ras 1 [Mus musculus] |
| 233 | 1 | 194 | 370 | 951 | g7008404 | 8.00E−75 | kappa B-ras 2 [Homo sapiens] |
| 234 | 2 | 222 | 17 | 682 | g9368448 | 1.00E−111 | phospholipase C-beta-1a [Homo sapiens] |
| 234 | 2 | 222 | 17 | 682 | g9368450 | 1.00E−111 | phospholipase C-beta-1b [Homo sapiens] |
| 234 | 2 | 222 | 17 | 682 | g206218 | 1.00E−110 | phospholipase C-1 [Rattus sp.] |
| 235 | 3 | 185 | 126 | 680 | g3599940 | 1.00E−57 | faciogenital dysplasia protein 2 [Mus musculus] |
| 235 | 3 | 185 | 126 | 680 | g10440426 | 8.00E−42 | FLJ00048 protein [Homo sapiens] |
| 235 | 3 | 185 | 126 | 680 | g595425 | 4.00E−20 | FGD1 [Homo sapiens] |
| 236 | 2 | 192 | 707 | 1282 | | | |
| 237 | 3 | 61 | 204 | 386 | | | |
| 238 | 2 | 335 | 17 | 1021 | g3005085 | 2.00E−92 | hook1 protein [Homo sapiens] |
| 238 | 2 | 335 | 17 | 1021 | g5706448 | 2.00E−92 | dJ782L23.1 (HOOK1) [Homo sapiens] |
| 238 | 2 | 335 | 17 | 1021 | g3005087 | 2.00E−70 | hook2 protein [Homo sapiens] |
| 239 | 1 | 346 | 1261 | 2298 | g1109782 | 1.00E−105 | protein-tyrosine phosphatase [Homo sapiens] |
| 239 | 1 | 346 | 1261 | 2298 | g1781037 | 1.00E−76 | neuronal tyrosine threonine phosphatase 1 [Mus musculus] |
| 239 | 1 | 346 | 1261 | 2298 | g10241798 | 5.00E−11 | hypothetical protein SCE41.24c [Streptomyces coelicolor] |
| 240 | 3 | 298 | 147 | 1040 | g4678722 | 1.00E−156 | hypothetical protein [Homo sapiens] |
| 240 | 3 | 298 | 147 | 1040 | g4007153 | 1.00E−153 | dJ272L16.1 (Rat Ca2+/Calmodulin dependent Protein Kinase LIKE protein) |
| | | | | | | | [Homo sapiens] |
| 240 | 3 | 298 | 147 | 1040 | g2077934 | 1.00E−152 | Protein Kinase [Rattus norvegicus] |
| 241 | 1 | 133 | 133 | 531 | g10440426 | 1.00E−34 | FLJ00048 protein [Homo sapiens] |
| 241 | 1 | 133 | 133 | 531 | g3599940 | 2.00E−16 | faciogenital dysplasia protein 2 [Mus musculus] |
| 242 | 2 | 354 | 821 | 1882 | g11907572 | 1.00E−143 | TSC22-related inducible leucine zipper 1b [Mus musculus] |
| 242 | 2 | 354 | 821 | 1882 | g1181619 | 1.00E−106 | a variant of TSC-22 [Gallus gallus] |
| 242 | 2 | 354 | 821 | 1882 | g3327152 | 9.00E−16 | KIAA0669 protein [Homo sapiens] |
| 243 | 1 | 237 | 1 | 711 | g6683492 | 1.00E−105 | bromodomain PHD finger transcription factor [Homo sapiens] |
| 243 | 1 | 237 | 1 | 711 | g3876452 | 9.00E−53 | contains similarity to Pfam domain: PF00439 (Bromodomain), Score = 125.5, E- |
| | | | | | | | value = 1.5e−35, N = 1; PF00628 (PHD-finger), Score = 102.0, |
| | | | | | | | E-value =3.8e−27, N = 2 [Caenorhabditis elegans] |
| 243 | 1 | 237 | 1 | 711 | g3876449 | 9.00E−53 | predicted using Genefinder˜contains similarity to Pfam domain: PF00439 |
| | | | | | | | (Bromodomain), Score = 125.5, E-value = 1.5e−35, N = 1; PF00628 |
| | | | | | | | (PHD-finger), Score = 102.0, E-value = 3.8e−27, N = 2 [Caenorhabditis elegans] |
| 244 | 1 | 161 | 1 | 483 | g6330736 | 1.00E−42 | KIAA1234 protein [Homo sapiens] |
| 244 | 1 | 161 | 1 | 483 | g11244871 | 1.00E−40 | dioxin receptor repressor [Homo sapiens] |
| 244 | 1 | 161 | 1 | 483 | g4164151 | 4.00E−35 | AhR repressor [Mus musculus] |
| 245 | 3 | 151 | 54 | 506 | g10433955 | 9.00E−44 | unnamed protein product [Homo sapiens] |
| 245 | 3 | 151 | 54 | 506 | g7295442 | 1.00E−16 | CG17334 gene product [Drosophila melanogaster] |
| 245 | 3 | 151 | 54 | 506 | g2745892 | 1.00E−12 | Y box transcription factor [Mus musculus] |
| 246 | 2 | 160 | 173 | 652 | g3924670 | 4.00E−68 | supported by Genscan and several ESTs: C83049 (NID: g3062006), |
| | | | | | | | AA823760 (NID: g2893628), AA215791 (NID: g1815572), AI095488 |
| | | | | | | | (NID: g3434464), and AA969095 (NID: g3144275) [Homo sapiens] |
| 246 | 2 | 160 | 173 | 652 | g5640105 | 2.00E−59 | homeobox protein LSX [Homo sapiens] |
| 246 | 2 | 160 | 173 | 652 | g6523391 | 6.00E−59 | phtf protein [Mus musculus] |
| 247 | 3 | 160 | 108 | 587 | g6939732 | 1.00E−52 | transcription factor Elongin A2 [Homo sapiens] |
| 247 | 3 | 160 | 108 | 587 | g4581412 | 1.00E−29 | dJ886K2.1 (elongin A; RNA polymerase; RNA polymerase II; RNA polymerase |
| | | | | | | | II elongation factor.) [Homo sapiens] |
| 247 | 3 | 160 | 108 | 587 | g992563 | 1.00E−29 | elongin A [Homo sapiens] |
| 248 | 1 | 171 | 25 | 537 | g11907923 | 4.00E−29 | enhancer of polycomb [Homo sapiens] |
| 248 | 1 | 171 | 25 | 537 | g3757890 | 3.00E−18 | enhancer of polycomb [Drosophila melanogaster] |
| 248 | 1 | 171 | 25 | 537 | g7303589 | 3.00E−18 | E(Pc) gene product [Drosophila melanogaster] |
| 249 | 2 | 449 | 266 | 1612 | g10443047 | 0 | bA465L10.2 (novel C2H2 type zinc finger protein similar to chicken FZF-1) |
| | | | | | | | [Homo sapiens] |
| 249 | 2 | 449 | 266 | 1612 | g10438918 | 0 | unnamed protein product [Homo sapiens] |
| 249 | 2 | 449 | 266 | 1612 | g984814 | 8.00E−98 | zinc finger protein [Gallus gallus] |
| 250 | 2 | 127 | 140 | 520 | g10434195 | 2.00E−64 | unnamed protein product [Homo sapiens] |
| 250 | 2 | 127 | 140 | 520 | g6467206 | 3.00E−36 | gonadotropin inducible transcription repressor-4 [Homo sapiens] |
| 250 | 2 | 127 | 140 | 520 | g6330394 | 4.00E−34 | KIAA1198 protein [Homo sapiens] |
| 251 | 1 | 157 | 1 | 471 | g340446 | 2.00E−17 | zinc finger protein 7 (ZFP7) [Homo sapiens] |
| 251 | 1 | 157 | 1 | 471 | g4325310 | 2.00E−17 | zinc-finger protein 7 [Homo sapiens] |
| 251 | 1 | 157 | 1 | 471 | g6007771 | 5.00E−17 | KID2 [Mus musculus] |
| 252 | 1 | 305 | 145 | 1059 | g6002480 | 3.00E−49 | BWSCR2 associated zinc-finger protein BAZ2 [Homo sapiens] |
| 252 | 1 | 305 | 145 | 1059 | g9963806 | 3.00E−47 | zinc finger protein ZNF287 [Homo sapiens] |
| 252 | 1 | 305 | 145 | 1059 | g11527849 | 8.00E−43 | zinc finger protein SKAT2 [Mus musculus] |
| 253 | 2 | 717 | 305 | 2455 | g10047335 | 0 | KIAA1629 protein [Homo sapiens] |
| 253 | 2 | 717 | 305 | 2455 | g1504006 | 1.00E−96 | similar to human ZFY protein. [Homo sapiens] |
| 253 | 2 | 717 | 305 | 2455 | g7243280 | 4.00E−66 | KIAA1441 protein [Homo sapiens] |
| 254 | 1 | 211 | 1 | 633 | g10047183 | 3.00E−49 | KIAA1559 protein [Homo sapiens] |
| 254 | 1 | 211 | 1 | 633 | g5080758 | 2.00E−45 | BC331191_1 [Homo sapiens] |
| 254 | 1 | 211 | 1 | 633 | g498721 | 3.00E−44 | zinc finger protein [Homo sapiens] |
| 255 | 2 | 103 | 2 | 310 | g498152 | 2.00E−20 | ha0946 protein is Kruppel-related. [Homo sapiens] |
| 255 | 2 | 103 | 2 | 310 | g7576272 | 2.00E−20 | bA393J16.1 (zinc finger protein 33a (KOX 31)) [Homo sapiens] |
| 255 | 2 | 103 | 2 | 310 | g10440081 | 2.00E−19 | unnamed protein product [Homo sapiens] |
| 256 | 3 | 84 | 135 | 386 | g347906 | 2.00E−26 | zinc finger protein [Homo sapiens] |
| 256 | 3 | 84 | 135 | 386 | g3342002 | 1.00E−25 | hematopoietic cell derived zinc finger protein [Homo sapiens] |
| 256 | 3 | 84 | 135 | 386 | g8163824 | 5.00E−25 | krueppel-like zinc finger protein HZF2 [Homo sapiens] |
| 257 | 1 | 194 | 103 | 684 | g10435738 | 4.00E−74 | unnamed protein product [Homo sapiens] |
| 257 | 1 | 194 | 103 | 684 | g1017722 | 8.00E−73 | repressor transcriptional factor [Homo sapiens] |
| 257 | 1 | 194 | 103 | 684 | g7959207 | 3.00E−71 | KIAA1473 protein [Homo sapiens] |
| 258 | 1 | 129 | 28 | 414 | g2072955 | 6.00E−07 | p40 [Homo sapiens] |
| 258 | 1 | 129 | 28 | 414 | g483915 | 8.00E−07 | ORF1, encodes a 40 kDa product [Homo sapiens] |
| 258 | 1 | 129 | 28 | 414 | g339776 | 8.00E−07 | ORF1 codes for a 40 kDa product [Homo sapiens] |
| 259 | 3 | 93 | 75 | 353 | g3329372 | 4.00E−36 | DNA-binding protein [Homo sapiens] |
| 259 | 3 | 93 | 75 | 353 | g7959207 | 1.00E−33 | KIAA1473 protein [Homo sapiens] |
| 259 | 3 | 93 | 75 | 353 | g184452 | 3.00E−33 | Krueppel-related DNA-binding protein [Homo sapiens] |
| 260 | 3 | 193 | 369 | 947 | g8099348 | 1.00E−38 | zinc finger protein [Homo sapiens] |
| 260 | 3 | 193 | 369 | 947 | g5730196 | 2.00E−38 | Kruppel-type zinc finger [Homo sapiens] |
| 260 | 3 | 193 | 369 | 947 | g8050899 | 4.00E−38 | ZNF180 [Homo sapiens] |
| 261 | 3 | 111 | 3 | 335 | g7023216 | 1.00E−14 | unnamed protein product [Homo sapiens] |
| 261 | 3 | 111 | 3 | 335 | g3406676 | 6.00E−14 | zinc finger protein 54 [Mus musculus] |
| 261 | 3 | 111 | 3 | 335 | g9802037 | 3.00E−13 | zinc finger protein SBZF3 [Homo sapiens] |
| 262 | 3 | 137 | 75 | 485 | g186774 | 1.00E−26 | zinc finger protein [Homo sapiens] |
| 262 | 3 | 137 | 75 | 485 | g2384653 | 6.00E−26 | Krueppel family zinc finger protein [Homo sapiens] |
| 262 | 3 | 137 | 75 | 485 | g8163824 | 6.00E−26 | krueppel-like zinc finger protein HZF2 [Homo sapiens] |
| 263 | 3 | 68 | 51 | 254 | g7239109 | 2.00E−15 | HSPC059 [Homo sapiens] |
| 263 | 3 | 68 | 51 | 254 | g347906 | 4.00E−15 | zinc finger protein [Homo sapiens] |
| 263 | 3 | 68 | 51 | 254 | g7023216 | 2.00E−14 | unnamed protein product [Homo sapiens] |
| 264 | 3 | 101 | 90 | 392 | g3329372 | 8.00E−35 | DNA-binding protein [Homo sapiens] |
| 264 | 3 | 101 | 90 | 392 | g4559318 | 7.00E−32 | BC273239_1 [Homo sapiens] |
| 264 | 3 | 101 | 90 | 392 | g184452 | 9.00E−32 | Krueppel-related DNA-binding protein [Homo sapiens] |
| 265 | 1 | 96 | 184 | 471 | g4589588 | 5.00E−22 | KIAA0972 protein [Homo sapiens] |
| 265 | 1 | 96 | 184 | 471 | g4514561 | 6.00E−22 | KRAB-containing zinc-finger protein KRAZ2 [Mus musculus] |
| 265 | 1 | 96 | 184 | 471 | g7576272 | 2.00E−21 | bA393J16.1 (zinc finger protein 33a (KOX 31)) [Homo sapiens] |
| 266 | 2 | 251 | 2 | 754 | g55471 | 1.00E−134 | Zfp-29 [Mus musculus] |
| 266 | 2 | 251 | 2 | 754 | g1020145 | 3.00E−73 | DNA binding protein [Homo sapiens] |
| 266 | 2 | 251 | 2 | 754 | g6002478 | 3.00E−72 | BWSCR2 associated zinc-finger protein BAZ1 [Homo sapiens] |
| 267 | 3 | 522 | 36 | 1601 | g10443047 | 0 | bA465L10.2 (novel C2H2 type zinc finger protein similar to chicken FZF-1) |
| | | | | | | | [Homo sapiens] |
| 267 | 3 | 522 | 36 | 1601 | g10438918 | 0 | unnamed protein product [Homo sapiens] |
| 267 | 3 | 522 | 36 | 1601 | g984814 | 2.00E−97 | zinc finger protein [Gallus gallus] |
| 268 | 2 | 267 | 2 | 802 | g9886891 | 4.00E−45 | zinc finger protein 276 C2H2 type [Mus musculus] |
| 268 | 2 | 267 | 2 | 802 | g11611571 | 3.00E−43 | hypothetical protein [Macaca fascicularis] |
| 268 | 2 | 267 | 2 | 802 | g453376 | 4.00E−43 | zinc finger protein PZF [Mus musculus] |
| 269 | 2 | 286 | 2 | 859 | g2754696 | 9.00E−08 | high molecular mass nuclear antigen [Gallus gallus] |
| 269 | 2 | 286 | 2 | 859 | g2078483 | 9.00E−06 | antifreeze glycopeptide AFGP polyprotein precursor [Boreogadus saida] |
| 270 | 3 | 194 | 270 | 851 | g8575782 | 1.00E−112 | PR-domain zinc finger protein 6 isoform A; PR-domain family protein 3 isoform |
| | | | | | | | A; PRDM6A; PFM3A [Homo sapiens] |
| 270 | 3 | 194 | 270 | 851 | g10437767 | 1.00E−26 | unnamed protein product [Homo sapiens] |
| 270 | 3 | 194 | 270 | 851 | g7295698 | 9.00E−26 | CG15436 gene product [Drosophila melanogaster] |
| 271 | 3 | 263 | 3 | 791 | g6409345 | 1.00E−107 | zinc finger protein ZNF180 [Homo sapiens] |
| 271 | 3 | 263 | 3 | 791 | g8050899 | 1.00E−107 | ZNF180 [Homo sapiens] |
| 271 | 3 | 263 | 3 | 791 | g200407 | 1.00E−101 | pMLZ-4 [Mus musculus] |
| 272 | 2 | 142 | 290 | 715 | g4062983 | 5.00E−65 | Eos protein [Mus musculus] |
| 272 | 2 | 142 | 290 | 715 | g9408382 | 4.00E−46 | eos [Raja eglanteria] |
| 272 | 2 | 142 | 290 | 715 | g11612390 | 3.00E−42 | zinc finger transcription factor Eos [Homo sapiens] |
| 273 | 2 | 164 | 2 | 493 | g1049301 | 3.00E−25 | KRAB zinc finger protein; Method: conceptual translation supplied by author |
| | | | | | | | [Homo sapiens] |
| 273 | 2 | 164 | 2 | 493 | g10047251 | 9.00E−25 | KIAA1588 protein [Homo sapiens] |
| 273 | 2 | 164 | 2 | 493 | g8809810 | 1.00E−19 | KRAB zinc finger protein [Mus musculus] |
| 274 | 2 | 107 | 509 | 829 | g1237278 | 2.00E−36 | zinc finger protein [Cavia porcellus] |
| 274 | 2 | 107 | 509 | 829 | g7023417 | 4.00E−36 | unnamed protein product [Homo sapiens] |
| 274 | 2 | 107 | 509 | 829 | g11917507 | 5.00E−36 | HPF1 protein [Homo sapiens] |
| 275 | 3 | 105 | 336 | 650 | g9801232 | 2.00E−51 | bA508N22.2 (zinc finger protein 37a (KOX 21)) [Homo sapiens] |
| 275 | 3 | 105 | 336 | 650 | g829151 | 2.00E−51 | ZNF37A [Homo sapiens] |
| 275 | 3 | 105 | 336 | 650 | g5730196 | 4.00E−36 | Kruppel-type zinc finger [Homo sapiens] |
| 276 | 1 | 149 | 1 | 447 | g7656698 | 3.00E−91 | Zinc finger protein 222 [Homo sapiens] |
| 276 | 1 | 149 | 1 | 447 | g6118381 | 3.00E−91 | zinc finger protein ZNF222 [Homo sapiens] |
| 276 | 1 | 149 | 1 | 447 | g6118383 | 1.00E−81 | zinc finger protein ZNF223 [Homo sapiens] |
| 277 | 3 | 101 | 90 | 392 | g3329372 | 1.00E−30 | DNA-binding protein [Homo sapiens] |
| 277 | 3 | 101 | 90 | 392 | g4559318 | 3.00E−29 | BC273239_1 [Homo sapiens] |
| 277 | 3 | 101 | 90 | 392 | g1124876 | 5.00E−29 | Krueppel-related DNA-binding protein [Homo sapiens] |
| 278 | 3 | 137 | 6 | 416 | g11062533 | 2.00E−46 | bA245E14.1 (novel zinc finger protein similar to ZFP47) [Homo sapiens] |
| 278 | 3 | 137 | 6 | 416 | g5640017 | 2.00E−46 | zinc finger protein ZFP113 [Mus musculus] |
| 278 | 3 | 137 | 6 | 416 | g186774 | 5.00E−46 | zinc finger protein [Homo sapiens] |
| 279 | 3 | 97 | 165 | 455 | g829151 | 2.00E−27 | ZNF37A [Homo sapiens] |
| 279 | 3 | 97 | 165 | 455 | g9801232 | 2.00E−27 | bA508N22.2 (zinc finger protein 37a (KOX 21)) [Homo sapiens] |
| 279 | 3 | 97 | 165 | 455 | g3702137 | 9.00E−20 | dJ733D15.1 (Zinc-finger protein) [Homo sapiens] |
| 280 | 2 | 97 | 182 | 472 | g9801232 | 4.00E−29 | bA508N22.2 (zinc finger protein 37a (KOX 21)) [Homo sapiens] |
| 280 | 2 | 97 | 182 | 472 | g829151 | 4.00E−29 | ZNF37A [Homo sapiens] |
| 280 | 2 | 97 | 182 | 472 | g200407 | 4.00E−21 | pMLZ-4 [Mus musculus] |
| 281 | 1 | 179 | 31 | 567 | g10442700 | 3.00E−61 | zinc-finger protein ZBRK1 [Homo sapiens] |
| 281 | 1 | 179 | 31 | 567 | g10435411 | 3.00E−61 | unnamed protein product [Homo sapiens] |
| 281 | 1 | 179 | 31 | 567 | g10954044 | 3.00E−61 | KRAB zinc finger protein ZFQR [Homo sapiens] |
| 282 | 3 | 87 | 369 | 629 | g8099348 | 2.00E−14 | zinc finger protein [Homo sapiens] |
| 282 | 3 | 87 | 369 | 629 | g498725 | 2.00E−14 | zinc finger protein [Homo sapiens] |
| 282 | 3 | 87 | 369 | 629 | g495568 | 2.00E−13 | zinc finger protein [Homo sapiens] |
| 283 | 2 | 172 | 2 | 517 | g6007771 | 4.00E−97 | KID2 [Mus musculus] |
| 283 | 2 | 172 | 2 | 517 | g2970038 | 2.00E−93 | HKL1 [Homo sapiens] |
| 283 | 2 | 172 | 2 | 517 | g205067 | 2.00E−93 | zinc finger protein [Rattus norvegicus] |
| 284 | 1 | 151 | 1 | 453 | g1806134 | 5.00E−57 | zinc finger protein [Mus musculus] |
| 284 | 1 | 151 | 1 | 453 | g538413 | 5.00E−57 | zinc finger protein [Mus musculus] |
| 284 | 1 | 151 | 1 | 453 | g186774 | 3.00E−55 | zinc finger protein [Homo sapiens] |
| 285 | 2 | 89 | 83 | 349 | g7023216 | 2.00E−18 | unnamed protein product [Homo sapiens] |
| 285 | 2 | 89 | 83 | 349 | g9802037 | 4.00E−16 | zinc finger protein SBZF3 [Homo sapiens] |
| 285 | 2 | 89 | 83 | 349 | g7239109 | 7.00E−15 | HSPC059 [Homo sapiens] |
| 286 | 2 | 146 | 62 | 499 | g2739353 | 7.00E−56 | ZNF91L [Homo sapiens] |
| 286 | 2 | 146 | 62 | 499 | g7959207 | 5.00E−50 | KIAA1473 protein [Homo sapiens] |
| 286 | 2 | 146 | 62 | 499 | g3342002 | 7.00E−50 | hematopoietic cell derived zinc finger protein [Homo sapiens] |
| 287 | 1 | 78 | 1 | 234 | g487785 | 4.00E−16 | zinc finger protein ZNF136 [Homo sapiens] |
| 287 | 1 | 78 | 1 | 234 | g5262560 | 7.00E−15 | hypothetical protein [Homo sapiens] |
| 287 | 1 | 78 | 1 | 234 | g10434856 | 9.00E−15 | unnamed protein product [Homo sapiens] |
| 288 | 3 | 126 | 78 | 455 | g9963804 | 4.00E−47 | zinc finger protein ZNF286 [Homo sapiens] |
| 288 | 3 | 126 | 78 | 455 | g5640017 | 2.00E−46 | zinc finger protein ZFP113 [Mus musculus] |
| 288 | 3 | 126 | 78 | 455 | g7020166 | 4.00E−46 | unnamed protein product [Homo sapiens] |
| 289 | 1 | 96 | 151 | 438 | g4589588 | 5.00E−22 | KIAA0972 protein [Homo sapiens] |
| 289 | 1 | 96 | 151 | 438 | g4514561 | 6.00E−22 | KRAB-containing zinc-finger protein KRAZ2 [Mus musculus] |
| 289 | 1 | 96 | 151 | 438 | g7576272 | 2.00E−21 | bA393J16.1 (zinc finger protein 33a (KOX 31)) [Homo sapiens] |
| 290 | 1 | 149 | 118 | 564 | g7959207 | 1.00E−26 | KIAA1473 protein [Homo sapiens] |
| 290 | 1 | 149 | 118 | 564 | g498736 | 3.00E−26 | zinc finger protein [Homo sapiens] |
| 290 | 1 | 149 | 118 | 564 | g4454678 | 4.00E−23 | zinc finger protein 4 [Homo sapiens] |
| 291 | 2 | 134 | 152 | 553 | g498152 | 1.00E−06 | ha0946 protein is Kruppel-related. [Homo sapiens] |
| 291 | 2 | 134 | 152 | 553 | g10440081 | 1.00E−06 | unnamed protein product [Homo sapiens] |
| 291 | 2 | 134 | 152 | 553 | g7576272 | 1.00E−06 | bA393J16.1 (zinc finger protein 33a (KOX 31)) [Homo sapiens] |
| 292 | 2 | 212 | 2 | 637 | g7656698 | 1.00E−133 | Zinc finger protein 222 [Homo sapiens] |
| 292 | 2 | 212 | 2 | 637 | g6118381 | 1.00E−133 | zinc finger protein ZNF222 [Homo sapiens] |
| 292 | 2 | 212 | 2 | 637 | g6118383 | 1.00E−122 | zinc finger protein ZNF223 [Homo sapiens] |
| 293 | 2 | 108 | 2 | 325 | g4567179 | 2.00E−33 | BC37295_1 [Homo sapiens] |
| 293 | 2 | 108 | 2 | 325 | g10434142 | 9.00E−31 | unnamed protein product [Homo sapiens] |
| 293 | 2 | 108 | 2 | 325 | g5817149 | 9.00E−31 | hypothetical protein [Homo sapiens] |
| 294 | 1 | 83 | 97 | 345 | g930123 | 9.00E−24 | zinc finger protein (583 AA) [Homo sapiens] |
| 294 | 1 | 83 | 97 | 345 | g487785 | 8.00E−23 | zinc finger protein ZNF136 [Homo sapiens] |
| 294 | 1 | 83 | 97 | 345 | g5262560 | 1.00E−22 | hypothetical protein [Homo sapiens] |
| 295 | 1 | 180 | 1 | 540 | g498719 | 2.00E−83 | zinc finger protein [Homo sapiens] |
| 295 | 1 | 180 | 1 | 540 | g3953593 | 3.00E−69 | Zinc finger protein s11-6 [Mus musculus] |
| 295 | 1 | 180 | 1 | 540 | g6467206 | 4.00E−68 | gonadotropin inducible transcription repressor-4 [Homo sapiens] |
| 296 | 3 | 97 | 57 | 347 | g9801232 | 3.00E−28 | bA508N22.2 (zinc finger protein 37a (KOX 21)) [Homo sapiens] |
| 296 | 3 | 97 | 57 | 347 | g829151 | 3.00E−28 | ZNF37A [Homo sapiens] |
| 296 | 3 | 97 | 57 | 347 | g881564 | 4.00E−20 | ZNF157 [Homo sapiens] |
| 297 | 1 | 217 | 421 | 1071 | g6331377 | 1.00E−131 | KIAA1285 protein [Homo sapiens] |
| 297 | 1 | 217 | 421 | 1071 | g1020145 | 6.00E−53 | DNA binding protein [Homo sapiens] |
| 297 | 1 | 217 | 421 | 1071 | g2224593 | 1.00E−52 | KIAA0326 [Homo sapiens] |
| 298 | 3 | 137 | 15 | 425 | g4456989 | 4.00E−20 | protease [Homo sapiens] |
| 298 | 3 | 137 | 15 | 425 | g9558703 | 4.00E−20 | protease [Homo sapiens] |
| 298 | 3 | 137 | 15 | 425 | g1780976 | 5.00E−20 | protease [Human endogenous retrovirus K] |
| 299 | 2 | 169 | 59 | 565 | g10434856 | 2.00E−40 | unnamed protein product [Homo sapiens] |
| 299 | 2 | 169 | 59 | 565 | g5262560 | 2.00E−40 | hypothetical protein [Homo sapiens] |
| 299 | 2 | 169 | 59 | 565 | g930123 | 1.00E−31 | zinc finger protein (583 AA) [Homo sapiens] |
| 300 | 3 | 135 | 3 | 407 | g10434856 | 3.00E−35 | unnamed protein product [Homo sapiens] |
| 300 | 3 | 135 | 3 | 407 | g5262560 | 3.00E−35 | hypothetical protein [Homo sapiens] |
| 300 | 3 | 135 | 3 | 407 | g10434195 | 2.00E−27 | unnamed protein product [Homo sapiens] |
| 301 | 1 | 170 | 22 | 531 | g10047297 | 2.00E−23 | KIAA1611 protein [Homo sapiens] |
| 301 | 1 | 170 | 22 | 531 | g7023216 | 2.00E−22 | unnamed protein product [Homo sapiens] |
| 301 | 1 | 170 | 22 | 531 | g347906 | 5.00E−16 | zinc finger protein [Homo sapiens] |
| 302 | 3 | 181 | 3 | 545 | g5931821 | 8.00E−79 | dJ228H13.3 (zinc finger protein) [Homo sapiens] |
| 302 | 3 | 181 | 3 | 545 | g6807587 | 8.00E−79 | hypothetical protein [Homo sapiens] |
| 302 | 3 | 181 | 3 | 545 | g488555 | 2.00E−63 | zinc finger protein ZNF135 [Homo sapiens] |
| 303 | 1 | 263 | 1 | 789 | g506502 | 1.00E−141 | NK10 [Mus musculus] |
| 303 | 1 | 263 | 1 | 789 | g488555 | 1.00E−92 | zinc finger protein ZNF135 [Homo sapiens] |
| 303 | 1 | 263 | 1 | 789 | g8453103 | 7.00E−88 | zinc finger protein [Homo sapiens] |
| 304 | 3 | 340 | 18 | 1037 | g7023216 | 1.00E−142 | unnamed protein product [Homo sapiens] |
| 304 | 3 | 340 | 18 | 1037 | g7023703 | 2.00E−89 | unnamed protein product [Homo sapiens] |
| 304 | 3 | 340 | 18 | 1037 | g10436789 | 7.00E−54 | unnamed protein product [Homo sapiens] |
| 305 | 1 | 89 | 103 | 369 | g7023216 | 2.00E−18 | unnamed protein product [Homo sapiens] |
| 305 | 1 | 89 | 103 | 369 | g9802037 | 4.00E−16 | zinc finger protein SBZF3 [Homo sapiens] |
| 305 | 1 | 89 | 103 | 369 | g7239109 | 7.00E−15 | HSPC059 [Homo sapiens] |
| 306 | 1 | 80 | 1 | 240 | g7959865 | 9.00E−20 | PRO2032 [Homo sapiens] |
| 306 | 1 | 80 | 1 | 240 | g8099520 | 6.00E−11 | muscleblind [Mus musculus] |
| 306 | 1 | 80 | 1 | 240 | g8515711 | 2.00E−10 | EXP35 [Homo sapiens] |
| 307 | 2 | 386 | 176 | 1333 | g3869259 | 0 | ZNF202 beta [Homo sapiens] |
| 307 | 2 | 386 | 176 | 1333 | g7328045 | 0 | hypothetical protein [Homo sapiens] |
| 307 | 2 | 386 | 176 | 1333 | g5360097 | 1.00E−123 | putative kruppel-related zinc finger protein NY-REN-23 antigen [Homo |
| | | | | | | | sapiens] |
| 308 | 2 | 368 | 71 | 1174 | g3882241 | 0 | KIAA0760 protein [Homo sapiens] |
| 308 | 2 | 368 | 71 | 1174 | g6760445 | 0 | Smad-and Olf-interacting zinc finger protein [Homo sapiens] |
| 308 | 2 | 368 | 71 | 1174 | g2149792 | 0 | Roaz [Rattus norvegicus] |
| 309 | 2 | 175 | 191 | 715 | g487787 | 8.00E−15 | zinc finger protein ZNF140 [Homo sapiens] |
| 309 | 2 | 175 | 191 | 715 | g10047183 | 9.00E−31 | KIAA1559 protein [Homo sapiens] |
| 309 | 2 | 175 | 191 | 715 | g4567179 | 2.00E−29 | BC37295_1 [Homo sapiens] |
| 310 | 2 | 78 | 521 | 754 | | | |
| 311 | 1 | 61 | 394 | 576 | | | |
| 312 | 1 | 73 | 172 | 390 | g2587027 | 4.00E−13 | HERV-E envelope glycoprotein [Homo sapiens] |
| 312 | 1 | 73 | 172 | 390 | g2587024 | 4.00E−13 | HERV-E envelope glycoprotein [Homo sapiens] |
| 312 | 1 | 73 | 172 | 390 | g1049232 | 2.00E−10 | HERV-E envelope protein [Human endogenous retrovirus] |
| 313 | 1 | 184 | 304 | 855 | g8132311 | 2.00E−74 | inwardly-rectifying potassium channel Kir5.1 [Homo sapiens] |
| 313 | 1 | 184 | 304 | 855 | g8132295 | 2.00E−74 | inwardly-rectifying potassium channel Kir5.1 [Homo sapiens] |
| 313 | 1 | 184 | 304 | 855 | g8132293 | 2.00E−74 | inwardly-rectifying potassium channel Kir5.1 [Homo sapiens] |
| 314 | 2 | 219 | 164 | 820 | g7105926 | 2.00E−22 | calcium channel alpha2-delta3 subunit [Homo sapiens] |
| 314 | 2 | 219 | 164 | 820 | g4186073 | 2.00E−22 | calcium channel alpha-2-delta-C subunit [Mus musculus] |
| 314 | 2 | 219 | 164 | 820 | g9929977 | 2.00E−22 | hypothetical protein [Macaca fascicularis] |
| 315 | 1 | 1603 | 1 | 4809 | g184039 | 0 | sodium channel alpha subunit [Homo sapiens] |
| 315 | 1 | 1603 | 1 | 4809 | g6782382 | 0 | voltage-gated sodium channel [Mus musculus] |
| 315 | 1 | 1603 | 1 | 4809 | g206858 | 0 | sodium channel alpha-subunit [Rattus norvegicus] |
| 316 | 3 | 200 | 240 | 839 | g913242 | 5.00E−71 | gamma-aminobutyric acid transporter type 3, GABA transporter type 3, GAT-3 |
| | | | | | | | [human, fetal brain, Peptide, 632 aa] [Homo sapiens] |
| 316 | 3 | 200 | 240 | 839 | g204220 | 2.00E−69 | beta-alanine-sensitive neuronal GABA transporter [Rattus norvegicus] |
| 316 | 3 | 200 | 240 | 839 | g202535 | 2.00E−69 | GABA transporter [Rattus norvegicus] |
| 317 | 3 | 329 | 3 | 989 | g6996442 | 4.00E−61 | CTL1 protein [Homo sapiens] |
| 317 | 3 | 329 | 3 | 989 | g6996589 | 1.00E−59 | CTL1 protein [Rattus norvegicus] |
| 317 | 3 | 329 | 3 | 989 | g6996587 | 2.00E−51 | CTL1 protein [Torpedo marmorata] |
| 318 | 3 | 256 | 3 | 770 | g5091520 | 1.00E−134 | ESTs AU058081(E30812),AU058365(E50679), AU030138(E50679) |
| | | | | | | | correspond to a region of the predicted gene.; Similar to Spinacia oleracea |
| | | | | | | | mRNA for proteasome 37 kD subunit.(X96974) [Oryza sativa] |
| 318 | 3 | 256 | 3 | 770 | g8096329 | 1.00E−134 | ESTs AU058081(E3082),AU075427(E30384) correspond to a region of the |
| | | | | | | | predicted gene.˜Similar to Spinacia oleracea proteasome 27 kD subunit |
| | | | | | | | (P52427) [Oryza sativa] |
| 318 | 3 | 256 | 3 | 770 | g8096319 | 1.00E−134 | ESTs AU058081(E3082),AU075427(E30384) correspond to a region of the |
| | | | | | | | predicted gene. ˜Similar to Spinacia oleracea proteasome 27 kD subunit |
| | | | | | | | (P52427) [Oryza sativa] |
| 319 | 2 | 76 | 2 | 229 | g951425 | 2.00E−07 | housekeeping protein [Rattus norvegicus] |
| 319 | 2 | 76 | 2 | 229 | g5759144 | 2.00E−07 | cyclophilin A [Mus musculus] |
| 319 | 2 | 76 | 2 | 229 | g50621 | 2.00E−07 | cyclophilin (AA 1-164) [Mus musculus] |
| 320 | 3 | 276 | 354 | 1181 | g7019854 | 1.00E−84 | unnamed protein product [Homo sapiens] |
| 320 | 3 | 276 | 354 | 1181 | g6567172 | 7.00E−84 | mDj10 [Mus musculus] |
| 320 | 3 | 276 | 354 | 1181 | g10436329 | 5.00E−81 | unnamed protein product [Homo sapiens] |
| 321 | 1 | 115 | 328 | 672 | g1049232 | 3.00E−24 | HERV-E envelope protein [Human endogenous retrovirus] |
| 321 | 1 | 115 | 328 | 672 | g2587024 | 2.00E−23 | HERV-E envelope glycoprotein [Homo sapiens] |
| 321 | 1 | 115 | 328 | 672 | g2587027 | 2.00E−23 | HERV-E envelope glycoprotein [Homo sapiens] |
| 322 | 3 | 227 | 3 | 683 | g2286123 | 6.00E−33 | testis specific DNAj-homolog [Mus musculus] |
| 322 | 3 | 227 | 3 | 683 | g6681592 | 1.00E−32 | DnaJ homolog [Homo sapiens] |
| 322 | 3 | 227 | 3 | 683 | g6648623 | 1.00E−32 | DNAJ homolog [Homo sapiens] |
| 323 | 3 | 100 | 153 | 452 | | | |
| 324 | 3 | 142 | 840 | 1265 | g2943716 | 5.00E−81 | 25 kDa trypsin inhibitor [Homo sapiens] |
| 324 | 3 | 142 | 840 | 1265 | g9885193 | 5.00E−54 | dJ881L22.3 (novel protein similar to a trypsin inhibitor) [Homo sapiens] |
| 324 | 3 | 142 | 840 | 1265 | g4324682 | 2.00E−52 | late gestation lung protein 1 [Rattus norvegicus] |
| 325 | 3 | 263 | 3 | 791 | g6957716 | 1.00E−135 | putative chaperonin [Arabidopsis thaliana] |
| 325 | 3 | 263 | 3 | 791 | g9755653 | 1.00E−132 | TCP-1 chaperonin-like protein [Arabidopsis thallana] |
| 325 | 3 | 263 | 3 | 791 | g5295933 | 2.00E−93 | chaperonin containing TCP-1 zeta-1 subunit [Mus musculus] |
| 326 | 2 | 357 | 23 | 1093 | g3882167 | 1.00E−171 | KIAA0723 protein [Homo sapiens] |
| 326 | 2 | 357 | 23 | 1093 | g9956070 | 1.00E−171 | similar to Homo sapiens mRNA for KIAA0723 protein with GenBank |
| | | | | | | | Accession Number AB018266.1 [] |
| 326 | 2 | 357 | 23 | 1093 | g6563246 | 1.00E−170 | matrin 3 [Homo sapiens] |
| 327 | 2 | 100 | 656 | 955 | | | |
| 328 | 2 | 303 | 2 | 910 | g8980660 | 1.00E−158 | proliferation-associated SNF2-like protein [Homo sapiens] |
| 328 | 2 | 303 | 2 | 910 | g805296 | 1.00E−149 | lymphocyte specific helicase [Mus musculus] |
| 328 | 2 | 303 | 2 | 910 | g9956001 | 8.00E−86 | similar to Mus musculus lymphocyte specific helicase mRNA with GenBank |
| | | | | | | | Accession Number U25691.1 [Homo sapiens] |
| 329 | 2 | 72 | 167 | 382 | | | |
| 330 | 2 | 76 | 80 | 307 | | | |
| 331 | 2 | 74 | 446 | 667 | g2104910 | 1.00E−29 | ORF derived from D1 leader region and integrase coding region [Homo |
| | | | | | | | sapiens] |
| 331 | 2 | 74 | 446 | 667 | g2104914 | 5.00E−21 | ORF derived from protease and integrase coding regions [Homo sapiens] |
| 331 | 2 | 74 | 446 | 667 | g4959374 | 5.00E−21 | pol protein [Homo sapiens] |
| 332 | 3 | 67 | 57 | 257 | | | |
| 333 | 2 | 192 | 302 | 877 | g8980660 | 8.00E−92 | proliferation-associated SNF2-like protein [Homo sapiens] |
| 333 | 2 | 192 | 302 | 877 | g9956001 | 8.00E−92 | similar to Mus musculus lymphocyte specific helicase mRNA with GenBank |
| | | | | | | | Accession Number U25691.1 [Homo sapiens] |
| 333 | 2 | 192 | 302 | 877 | g7022306 | 1.00E−89 | unnamed protein product [Homo sapiens] |
| 334 | 2 | 74 | 446 | 667 | g2104910 | 1.00E−30 | ORF derived from D1 leader region and integrase coding region [Homo |
| | | | | | | | sapiens] |
| 334 | 2 | 74 | 446 | 667 | g2104914 | 5.00E−21 | ORF derived from protease and integrase coding regions [Homo sapiens] |
| 334 | 2 | 74 | 446 | 667 | g4959374 | 5.00E−21 | pol protein [Homo sapiens] |
| 335 | 2 | 72 | 167 | 382 | | | |
| 336 | 2 | 55 | 557 | 721 | g2231380 | 8.00E−12 | orf; encodes putative chimeric protein with SET domain in N-terminus with |
| | | | | | | | similarity to several other human, Drosophlla, nematode and yeast proteins |
| | | | | | | | [Homo sapiens] |
| 336 | 2 | 55 | 557 | 721 | g3005702 | 8.00E−12 | unknown [Homo sapiens] |
| 336 | 2 | 55 | 557 | 721 | g1263081 | 1.00E−11 | mariner transposase [Homo sapiens] |
| 337 | 3 | 107 | 1614 | 1934 | | | |
| 338 | 3 | 147 | 63 | 503 | g10047265 | 7.00E−81 | KIAA1595 protein [Homo sapiens] |
| 338 | 3 | 147 | 63 | 503 | g10176757 | 3.00E−26 | ATP-dependent RNA helicase-like protein [Arabidopsis thaliana] |
| 338 | 3 | 147 | 63 | 503 | g3776011 | 3.00E−26 | RNA helicase [Arabidopsis thaliana] |
| 339 | 1 | 257 | 199 | 969 | g10434055 | 1.00E−128 | unnamed protein product [Homo sapiens] |
| 339 | 1 | 257 | 199 | 969 | g7243213 | 1.00E−126 | KIAA1416 protein [Homo sapiens] |
| 339 | 1 | 257 | 199 | 969 | g11345539 | 1.00E−120 | dJ620E11.1 (novel Helicase C-terminal domain and SNF2 N-terminal domains |
| | | | | | | | containing protein, similar to KIAA0308) [Homo sapiens] |
| 340 | 3 | 63 | 3 | 191 | | | |
| 341 | 1 | 112 | 1639 | 1974 | | | |
| 342 | 3 | 427 | 2097 | 3377 | g2599502 | 0 | protocadherin 68 [Homo sapiens] |
| 342 | 3 | 427 | 2097 | 3377 | g7243181 | 4.00E−49 | KIAA1400 protein [Homo sapiens] |
| 342 | 3 | 427 | 2097 | 3377 | g4099551 | 5.00E−48 | OL-protocadherin [Mus musculus] |
| 343 | 2 | 144 | 635 | 1066 | g10436424 | 1.00E−10 | unnamed protein product [Homo sapiens] |
| 344 | 2 | 97 | 557 | 847 | | | |
| 345 | 3 | 75 | 675 | 899 | g2587027 | 4.00E−13 | HERV-E envelope glycoprotein [Homo sapiens] |
| 345 | 3 | 75 | 675 | 899 | g2587024 | 4.00E−13 | HERV-E envelope glycoprotein [Homo sapiens] |
| 345 | 3 | 75 | 675 | 899 | g1049232 | 2.00E−10 | HERV-E envelope protein [Human endogenous retrovirus] |
| 346 | 3 | 135 | 399 | 803 | g9368839 | 2.00E−71 | hypothetical protein [Homo sapiens] |
| 346 | 3 | 135 | 399 | 803 | g2739452 | 6.00E−58 | ribosomal protein L23A [Homo sapiens] |
| 346 | 3 | 135 | 399 | 803 | g1399086 | 6.00E−58 | ribosomal protein L23a [Homo sapiens] |
| 347 | 2 | 55 | 179 | 343 | | | |
| 348 | 2 | 129 | 425 | 811 | g11493463 | 2.00E−22 | PRO2852 [Homo sapiens] |
| 348 | 2 | 129 | 425 | 811 | g9280152 | 5.00E−22 | unnamed portein product [Macaca fascicularis] |
| 348 | 2 | 129 | 425 | 811 | g10437485 | 5.00E−21 | unnamed protein product [Homo sapiens] |
| 349 | 2 | 291 | 122 | 994 | g673417 | 1.00E−152 | class II antigen [Homo sapiens] |
| 349 | 2 | 291 | 122 | 994 | g703089 | 1.00E−152 | MHC class II DP3-alpha [Homo sapiens] |
| 349 | 2 | 291 | 122 | 994 | g758100 | 1.00E−137 | SB classII histocompatibility antigen alpha- chain [Homo sapiens] |
| 350 | 1 | 517 | 1 | 1551 | g402843 | 1.00E−144 | cytochrome P450 2B-Bx = phenobarbital-inducible [rabbits, kidney, Peptide, 491 |
| | | | | | | | aa] [Oryctolagus cuniculus] |
| 350 | 1 | 517 | 1 | 1551 | g404777 | 1.00E−144 | cytochrome P-450 2B-Bx [Oryctolagus cuniculus] |
| 350 | 1 | 517 | 1 | 1551 | g164959 | 1.00E−142 | cytochrome P-450 [Oryctolagus cuniculus] |
| 351 | 1 | 232 | 1300 | 1995 | | | |
| 352 | 1 | 220 | 67 | 726 | g11863734 | 2.00E−80 | dJ857M17.2 (novel protein similar to cytochrome c oxidase subunit IV |
| | | | | | | | (COX4)) [Homo sapiens] |
| 352 | 1 | 220 | 67 | 726 | g8809758 | 9.00E−42 | cytochrome c oxidase subunit IV isoform 2 precursor [Thunnus obesus] |
| 352 | 1 | 220 | 67 | 726 | g2809498 | 3.00E−41 | cytochrome c oxidase subunit IV [Gorilla gorilla] |
| 353 | 1 | 95 | 1 | 285 | | | |
| 354 | 2 | 331 | 2 | 994 | g11229985 | 1.00E−176 | unnamed protein product [Homo sapiens] |
| 354 | 2 | 331 | 2 | 994 | g11229992 | 6.00E−57 | unnamed protein product [Mus musculus] |
| 354 | 2 | 331 | 2 | 994 | g30095 | 6.00E−49 | collagen subunit (alpha-1 (X)) 3 [Homo sapiens] |
| 355 | 3 | 93 | 54 | 332 | g11177164 | 4.00E−12 | polydom protein [Mus musculus] |
| 355 | 3 | 93 | 54 | 332 | g391669 | 4.00E−07 | hikaru genki type4 product precursor [Drosophila melanogaster] |
| 355 | 3 | 93 | 54 | 332 | g391667 | 4.00E−07 | hikaru genki type3 product precursor [Drosophila melanogaster] |
| 356 | 1 | 112 | 1 | 336 | | | |
| 357 | 3 | 73 | 192 | 410 | | | |
| 358 | 1 | 239 | 181 | 897 | g4582324 | 1.00E−129 | dJ708F5.1 (PUTATIVE novel Collagen alpha 1 LIKE protein) [Homo sapiens] |
| 358 | 1 | 239 | 181 | 897 | g1732121 | 4.00E−36 | cartilage matrix protein [Homo sapiens] |
| 358 | 1 | 239 | 181 | 897 | g180654 | 2.00E−35 | cartilage matrix protein [Homo sapiens] |
| 359 | 1 | 528 | 4 | 1587 | g1903218 | 0 | type II intermediate filament of hair keratin [Homo sapiens] |
| 359 | 1 | 528 | 4 | 1587 | g7161771 | 0 | keratin [Homo sapiens] |
| 359 | 1 | 528 | 4 | 1587 | g4103156 | 0 | hair keratin basic 5; keratin Hb5 [Mus musculus] |
| 360 | 2 | 157 | 161 | 631 | g11034725 | 2.00E−64 | hNBL4 [Homo sapiens] |
| 360 | 2 | 157 | 161 | 631 | g466548 | 3.00E−63 | NBL4 [Mus musculus] |
| 360 | 2 | 157 | 161 | 631 | g2822458 | 5.00E−54 | band 4.1-like protein 4 [Danio rerio] |
| 361 | 3 | 65 | 54 | 248 | g3724141 | 6.00E−08 | myosin I [Rattus norvegicus] |
| 361 | 3 | 65 | 54 | 248 | g3882175 | 6.00E−08 | KIAA0727 protein [Homo sapiens] |
| 362 | 3 | 517 | 3 | 1553 | g6855339 | 1.00E−120 | dJ111C20.1 (similar to Chlamydomonas radial spoke protein 3) [Homo |
| | | | | | | | sapiens] |
| 362 | 3 | 517 | 3 | 1553 | g18218 | 1.00E−75 | spoke protein [Chlamydomonas reinhardtii] |
| 362 | 3 | 517 | 3 | 1553 | g7295323 | 9.00E−47 | CG10099 gene product [Drosophila melanogaster] |
| 363 | 2 | 60 | 314 | 493 | | | |
| 364 | 1 | 239 | 127 | 843 | g1813638 | 9.00E−53 | PF20 [Chlamydomonas reinhardtii] |
| 364 | 1 | 239 | 127 | 843 | g3983133 | 2.00E−47 | pf20 homolog [Trypanosoma brucei] |
| 364 | 1 | 239 | 127 | 843 | g607003 | 1.00E−37 | beta transducin-like protein [Podospora anserina] |
| 365 | 1 | 160 | 1 | 480 | | | |
| 366 | 3 | 757 | 3 | 2273 | g8896164 | 0 | kinesin-like protein GAKIN [Homo sapiens] |
| 366 | 3 | 757 | 3 | 2273 | g10697238 | 0 | KIF13A [Mus musculus] |
| 366 | 3 | 757 | 3 | 2273 | g11761613 | 0 | kinesin-like protein RBKIN2 [Homo sapiens] |
| 367 | 3 | 162 | 3 | 488 | g11231085 | 1.00E−56 | hypothetical protein [Macaca fascicularis] |
| 367 | 3 | 162 | 3 | 488 | g7385113 | 2.00E−18 | ankyrin 1 [Bos taurus] |
| 367 | 3 | 162 | 3 | 488 | g747710 | 2.00E−18 | alt, ankyrin (variant 2.2) [Homo sapiens] |
| 368 | 2 | 635 | 308 | 2212 | g1353782 | 0 | dystrophin-related protein 2 [Homo sapiens] |
| 368 | 2 | 635 | 308 | 2212 | g11066165 | 0 | dystrophin-related protein 2 A-form splice variant [Rattus norvegicus] |
| 368 | 2 | 635 | 308 | 2212 | g11066167 | 0 | dystrophin-related protein 2 B-form splice variant [Rattus norvegicus] |
| 369 | 3 | 433 | 999 | 2297 | g190752 | 0 | pemphigus vulgaris antigen [Homo sapiens] |
| 369 | 3 | 433 | 999 | 2297 | g2290200 | 1.00E−176 | desmoglein 3 [Mus musculus] |
| 369 | 3 | 433 | 999 | 2297 | g416178 | 2.00E−58 | desmoglein 2 [Homo sapiens] |
| 370 | 3 | 531 | 3 | 1595 | g28969 | 7.00E−71 | 64 Kd autoantigen [Homo sapiens] |
| 370 | 3 | 531 | 3 | 1595 | g6934240 | 8.00E−61 | tropomodulin 2 [Homo sapiens] |
| 370 | 3 | 531 | 3 | 1595 | g7288857 | 3.00E−60 | neural tropomodulin N-Tmod [Mus musculus] |
| 371 | 2 | 257 | 383 | 1153 | g1469868 | 1.00E−124 | The KIAA0143 gene product is related to a putative C.elegans gene encoded |
| | | | | | | | on cosmid C32D5. [Homo sapiens] |
| 371 | 2 | 257 | 383 | 1153 | g4589550 | 4.00E−82 | KIAA0953 protein [Homo sapiens] |
| 371 | 2 | 257 | 383 | 1153 | g7304005 | 1.00E−55 | cmp44E gene product [alt 1] [Drosophila melanogaster] |
| 372 | 1 | 242 | 139 | 864 | g387514 | 1.00E−123 | DM-20 protein [Mus musculus] |
| 372 | 1 | 242 | 139 | 864 | g190088 | 1.00E−123 | DM-20 [Homo sapiens] |
| 372 | 1 | 242 | 139 | 864 | g200409 | 1.00E−122 | proteolipid protein variant Dm-20 [Mus musculus] |
| 373 | 2 | 60 | 380 | 559 | | | |
| 374 | 1 | 157 | 22 | 492 | g7268562 | 1.00E−59 | ribosomal protein L32-like protein [Arabidopsis thaliana] |
| 374 | 1 | 157 | 22 | 492 | g5816996 | 1.00E−59 | ribosomal protein L32-like protein [Arabidopsis thaliana] |
| 374 | 1 | 157 | 22 | 492 | g10177580 | 7.00E−59 | ribosomal protein L32 [Arabidopsis thaliana] |
| 375 | 3 | 158 | 3 | 476 | g643074 | 4.00E−76 | putative 40S ribosomal protein s12 [Fragaria x ananassa] |
| 375 | 3 | 158 | 3 | 476 | g6716785 | 1.00E−75 | 40s ribosomal protein S23 [Euphorbia esula] |
| 375 | 3 | 158 | 3 | 476 | g7413571 | 6.00E−75 | putative protein [Arabidopsis thaliana] |
| 376 | 2 | 238 | 14 | 727 | g10799832 | 1.00E−93 | ribosomal protein L11-like [Nicotiana tabacum] |
| 376 | 2 | 238 | 14 | 727 | g7630065 | 4.00E−93 | ribosomal protein L11-like [Arabidopsis thaliana] |
| 376 | 2 | 238 | 14 | 727 | g11908058 | 4.00E−93 | ribosomal protein L11, cytosolic [Arabidopsis thaliana] |
| 377 | 3 | 102 | 3 | 308 | g57131 | 7.00E−41 | ribosomal protein S26 [Rattus norvegicus] |
| 377 | 3 | 102 | 3 | 308 | g296452 | 7.00E−41 | ribosomal protein S26 [Homo sapiens] |
| 377 | 3 | 102 | 3 | 308 | g3335024 | 7.00E−41 | ribosomal protein S26 [Homo sapiens] |
| 378 | 1 | 102 | 316 | 621 | g6969165 | 6.00E−53 | dJ475N16.3 (novel protein similar to RPL7A (60S ribosomal protein L7A)) |
| | | | | | | | [Homo sapiens] |
| 378 | 1 | 102 | 316 | 621 | g6687301 | 2.00E−21 | 60S ribosomal protein L7 [Cyanophora paradoxa] |
| 378 | 1 | 102 | 316 | 621 | g200785 | 1.00E−20 | ribosomal protein L7 [Mus musculus] |
| 379 | 3 | 177 | 3 | 533 | g206736 | 1.00E−82 | ribosomal protein L7 [Rattus norvegicus] |
| 379 | 3 | 177 | 3 | 533 | g200785 | 2.00E−80 | ribosomal protein L7 [Mus musculus] |
| 379 | 3 | 177 | 3 | 533 | g554269 | 2.00E−80 | ribosomal protein L7 [Mus musculus] |
| 380 | 2 | 86 | 257 | 514 | g550025 | 2.00E−31 | ribosomal protein S10 [Homo sapiens] |
| 380 | 2 | 86 | 257 | 514 | g57127 | 3.00E−30 | ribosomal protein S10 (AA 1-165) [Rattus norvegicus] |
| 380 | 2 | 86 | 257 | 514 | g9581772 | 3.00E−29 | bA371L19.2 (similar to ribosomal protein S10) [Homo sapiens] |
| 381 | 1 | 97 | 286 | 576 | g36140 | 2.00E−31 | ribosomal protein L7 [Homo sapiens] |
| 381 | 1 | 97 | 286 | 576 | g307388 | 2.00E−31 | ribosomal protein L7 [Homo sapiens] |
| 381 | 1 | 97 | 286 | 576 | g1335288 | 2.00E−31 | ribosomal protein L7 [Homo sapiens] |
| 382 | 1 | 82 | 70 | 315 | g409074 | 2.00E−19 | HBp15/L22 [Sus scrofa] |
| 382 | 1 | 82 | 70 | 315 | g409072 | 2.00E−19 | HBp15/L22 [Mus musculus] |
| 382 | 1 | 82 | 70 | 315 | g409070 | 2.00E−19 | HBp15/L22 [Homo sapiens] |
| 383 | 1 | 180 | 46 | 585 | g4886269 | 2.00E−75 | putative ribosomal protein S14 [Arabidopsis thaliana] |
| 383 | 1 | 180 | 46 | 585 | g6006890 | 6.00E−75 | putative 40S ribosomal protein s14; 67401-66292 [Arabidopsis thaliana] |
| 383 | 1 | 180 | 46 | 585 | g4678226 | 3.00E−74 | 40S ribosomal protein S14 [Arabidopsis thaliana] |
| 384 | 3 | 118 | 21 | 374 | g643074 | 2.00E−49 | putative 40S ribosomal protein s12 [Fragaria x ananassa] |
| 384 | 3 | 118 | 21 | 374 | g6716785 | 6.00E−49 | 40s ribosomal protein S23 [Euphorbia esula] |
| 384 | 3 | 118 | 21 | 374 | g7413571 | 3.00E−48 | putative protein [Arabidopsis thaliana] |
| 385 | 2 | 164 | 2 | 493 | g643074 | 4.00E−76 | putative 40S ribosomal protein s12 [Fragaria x ananassa] |
| 385 | 2 | 164 | 2 | 493 | g6716785 | 1.00E−75 | 40s ribosomal protein S23 [Euphorbia esula] |
| 385 | 2 | 164 | 2 | 493 | g7413571 | 6.00E−75 | putative protein [Arabidopsis thaliana] |
| 386 | 3 | 101 | 3 | 305 | g36130 | 1.00E−22 | ribosomal protein L31 (AA 1-125) [Homo sapiens] |
| 386 | 3 | 101 | 3 | 305 | g1655596 | 1.00E−22 | ribosomal protein L31 [Homo sapiens] |
| 386 | 3 | 101 | 3 | 305 | g57115 | 1.00E−22 | ribosomal protein L31 (AA 1-125) [Rattus norvegicus] |
| 387 | 3 | 259 | 3 | 779 | g2331301 | 1.00E−122 | ribosomal protein S4 type I [Zea mays] |
| 387 | 3 | 259 | 3 | 779 | g2345154 | 1.00E−120 | ribsomal protein S4 [Zea mays] |
| 387 | 3 | 259 | 3 | 779 | g7546687 | 1.00E−116 | ribosomal protein S4 [Arabidopsis thaliana] |
| 388 | 2 | 184 | 2 | 553 | g2668748 | 1.00E−95 | ribosomal protein L17 [Zea mays] |
| 388 | 2 | 184 | 2 | 553 | g19104 | 8.00E−85 | ribosomal protein L17-2 [Hordeum vulgare] |
| 388 | 2 | 184 | 2 | 553 | g19102 | 1.00E−82 | ribosomal protein L17-1 [Hordeum vulgare] |
| 389 | 2 | 152 | 2 | 457 | g338447 | 5.00E−28 | RPS16 [Homo sapiens] |
| 389 | 2 | 152 | 2 | 457 | g57714 | 5.00E−28 | ribosomal protein S16 (AA 1-146) [Rattus rattus] |
| 389 | 2 | 152 | 2 | 457 | g200796 | 2.00E−27 | 16S ribosomal protein [Mus musculus] |
| 390 | 3 | 158 | 3 | 476 | g643074 | 4.00E−76 | putative 40S ribosomal protein s12 [Fragaria x ananassa] |
| 390 | 3 | 158 | 3 | 476 | g6716785 | 1.00E−75 | 40s ribosomal protein S23 [Euphorbia esula] |
| 390 | 3 | 158 | 3 | 476 | g7413571 | 6.00E−75 | putative protein [Arabidopsis thaliana] |
| 391 | 1 | 94 | 34 | 315 | | | |
| 392 | 3 | 83 | 303 | 551 | g57121 | 3.00E−18 | ribosomal protein L37 [Rattus norvegicus] |
| 392 | 3 | 83 | 303 | 551 | g292441 | 3.00E−18 | ribosomal protein L37 [Homo sapiens] |
| 392 | 3 | 83 | 303 | 551 | g1839334 | 3.00E−18 | ribosomal protein L37 {C2-C2 zinc-finger-like} [human, HeLa cells, Peptlde, 97 |
| | | | | | | | aa] [Homo sapiens] |
| 393 | 2 | 174 | 2 | 523 | g10433651 | 3.00E−80 | unnamed protein product [Homo sapiens] |
| 393 | 2 | 174 | 2 | 523 | g10434617 | 3.00E−80 | unnamed protein product [Homo sapiens] |
| 393 | 2 | 174 | 2 | 523 | g545998 | 6.00E−79 | tricarboxylate carrier [Rattus sp.] |
| 394 | 3 | 183 | 3 | 551 | | | |
| 395 | 1 | 399 | 1 | 1197 | g11907599 | 0 | protein kinase HIPK2 [Homo sapiens] |
| 395 | 1 | 399 | 1 | 1197 | g5815141 | 0 | nuclear body associated kinase 1b [Mus musculus] |
| 395 | 1 | 399 | 1 | 1197 | g5815139 | 0 | nuclear body associated kinase 1a [Mus musculus] |
| 396 | 1 | 301 | 109 | 1011 | g7688667 | 1.00E−161 | PC326 protein [Homo sapiens] |
| 396 | 1 | 301 | 109 | 1011 | g2734854 | 1.00E−08 | Mus musculus Dentin Matrix Protein 1 [] |
| 396 | 1 | 301 | 109 | 1011 | g6137020 | 1.00E−08 | dentin matrix protein-1 [Mus musculus] |
| 397 | 2 | 105 | 2 | 316 | g178281 | 1.00E−47 | AHNAK nucleoprotein [Homo sapiens] |
| 397 | 2 | 105 | 2 | 316 | g50675 | 2.00E−47 | desmoyokin [Mus musculus] |
| 397 | 2 | 105 | 2 | 316 | g897824 | 5.00E−47 | AHNAK gene product [Homo sapiens] |
| 398 | 1 | 153 | 202 | 660 | g183233 | 1.00E−34 | beta-glucuronidase precursor (EC 3.2.1.31) [Homo sapiens] |
| 398 | 1 | 153 | 202 | 660 | g3549609 | 2.00E−33 | beta-glucuronidase [Cercopithecus aethiops] |
| 398 | 1 | 153 | 202 | 660 | g4102553 | 3.00E−29 | mutant beta-glucuronidase [Felis catus] |
| 399 | 1 | 161 | 106 | 588 | g7022046 | 1.00E−36 | unnamed protein product [Homo sapiens] |
| 399 | 1 | 161 | 106 | 588 | g7670456 | 5.00E−34 | unnamed protein product [Mus musculus] |
| 399 | 1 | 161 | 106 | 588 | g8671586 | 1.00E−29 | ataxin 2-binding protein [Homo sapiens] |
| 400 | 1 | 153 | 205 | 663 | g183233 | 1.00E−34 | beta-glucuronidase precursor (EC 3.2.1.31) [Homo sapiens] |
| 400 | 1 | 153 | 205 | 663 | g3549609 | 2.00E−33 | beta-glucuronidase [Cercopithecus aethiops] |
| 400 | 1 | 153 | 205 | 663 | g4102553 | 3.00E−29 | mutant beta-glucuronidase [Felis catus] |
| 401 | 3 | 135 | 651 | 1055 | g414797 | 9.00E−58 | pyruvate dehydrogenase phosphatase [Bos taurus] |
| 401 | 3 | 135 | 651 | 1055 | g3298607 | 3.00E−56 | pyruvate dehydrogenase phosphatase isoenzyme 1 [Rattus norvegicus] |
| 401 | 3 | 135 | 651 | 1055 | g7688679 | 3.00E−53 | pyruvate dehydrogenase [Homo sapiens] |
| 402 | 3 | 129 | 30 | 416 | | | |
| 403 | 1 | 299 | 1 | 897 | g440878 | 1.00E−149 | onconeural ventral antigen-1 [Homo sapiens] |
| 403 | 1 | 299 | 1 | 897 | g7025507 | 1.00E−137 | ventral neuron-specific protein 1 NOVA1 [Mus musculus] |
| 403 | 1 | 299 | 1 | 897 | g2673961 | 9.00E−99 | astrocytic NOVA-like RNA-binding protein [Homo sapiens] |
| 404 | 1 | 142 | 1 | 426 | g4105111 | 1.00E−43 | dehydrin 6 [Hordeum vulgare] |
| 404 | 1 | 142 | 1 | 426 | g6017938 | 4.00E−43 | dehydrin; DHN6 [Hordeum vulgare] |
| 404 | 1 | 142 | 1 | 426 | g5738195 | 1.00E−28 | abscisic acid response protein [Prunus dulcis] |
| 405 | 2 | 168 | 2 | 505 | g453189 | 9.00E−59 | acyl carrier protein [Zea mays] |
| 405 | 2 | 168 | 2 | 505 | g166971 | 4.00E−49 | acyl carrier protein III [Hordeum vulgare] |
| 405 | 2 | 168 | 2 | 505 | g166969 | 6.00E−41 | acyl carrier protein II [Hordeum vulgare] |
| 406 | 2 | 117 | 2 | 352 | g203923 | 1.00E−40 | diazepam binding inhibitor [Rattus norvegicus] |
| 406 | 2 | 117 | 2 | 352 | g1228089 | 1.00E−40 | multifunctional acyl-CoA-binding protein [Rattus norvegicus] |
| 406 | 2 | 117 | 2 | 352 | g203925 | 1.00E−40 | diazepam binding inhibitor [Rattus norvegicus] |
| 407 | 3 | 804 | 3 | 2414 | g10953883 | 0 | ubiquitin E3 ligase SMURF2 [Homo sapiens] |
| 407 | 3 | 804 | 3 | 2414 | g10047327 | 0 | KIAA1625 protein [Homo sapiens] |
| 407 | 3 | 804 | 3 | 2414 | g6446606 | 0 | E3 ubiquitin ligase SMURF1 [Homo sapiens] |
| 408 | 1 | 220 | 244 | 903 | g9622856 | 9.00E−24 | sorting nexin 15A [Homo sapiens] |
| 408 | 1 | 220 | 244 | 903 | g2529709 | 1.00E−23 | unknown [Homo sapiens] |
| 408 | 1 | 220 | 244 | 903 | g9622854 | 1.00E−23 | sorting nexin 15 [Homo sapiens] |
| 409 | 2 | 168 | 80 | 583 | g5823961 | 2.00E−87 | dJ20B11.1 (ortholog of rat RSEC5 (mammalian exocyst complex subunit)) |
| | | | | | | | [Homo sapiens] |
| 409 | 2 | 168 | 80 | 583 | g2827158 | 2.00E−84 | rsec5 [Rattus norvegicus] |
| 409 | 2 | 168 | 80 | 583 | g7295804 | 8.00E−29 | CG8843 gene product [Drosophila melanogaster] |
| 410 | 2 | 108 | 194 | 517 | g9963839 | 1.00E−50 | lipase [Homo sapiens] |
| 411 | 1 | 314 | 277 | 1218 | g3243240 | 4.00E−56 | syntaxin 11 [Homo sapiens] |
| 411 | 1 | 314 | 277 | 1218 | g4104685 | 1.00E−53 | syntaxin 11 [Homo sapiens] |
| 411 | 1 | 314 | 277 | 1218 | g3248918 | 8.00E−46 | syntaxin 11 [Homo sapiens] |
| 412 | 2 | 143 | 212 | 640 | g4512103 | 3.00E−57 | rab11 binding protein [Bos taurus] |
| 412 | 2 | 143 | 212 | 640 | g6049150 | 8.00E−43 | WD-containing protein [Rattus norvegicus] |
| 413 | 1 | 122 | 1 | 366 | | | |
| 414 | 2 | 86 | 623 | 880 | | | |
| 415 | 3 | 213 | 183 | 821 | | | |
| 416 | 1 | 263 | 40 | 828 | g9558701 | 3.00E−31 | gag [Homo sapiens] |
| 416 | 1 | 263 | 40 | 828 | g5802824 | 3.00E−31 | Gag-Pro-Pol protein [Homo sapiens] |
| 416 | 1 | 263 | 40 | 828 | g5802821 | 3.00E−31 | Gag-Pro-Pol protein [Homo sapiens] |
| 417 | 1 | 175 | 940 | 1464 | g246483 | 1.00E−63 | prohibitin [human, Peptide, 272 aa] [Homo sapiens] |
| 417 | 1 | 175 | 940 | 1464 | g206384 | 2.00E−63 | prohibitin [Rattus norvegicus] |
| 417 | 1 | 175 | 940 | 1464 | g541732 | 2.00E−63 | prohibitin or B-cell receptor associated protein (BAP) 32 [Mus musculus] |
| 418 | 2 | 272 | 167 | 982 | g505033 | 6.00E−75 | mitogen inducible gene mlg-2 [Homo sapiens] |
| 418 | 2 | 272 | 167 | 982 | g10727293 | 5.00E−33 | CG14991 gene product [alt 2] [Drosophila melanogaster] |
| 418 | 2 | 272 | 167 | 982 | g7292434 | 5.00E−33 | CG14991 gene product [alt 1] [Drosophila melanogaster] |
| 419 | 1 | 167 | 16 | 516 | g2587027 | 3.00E−34 | HERV-E envelope glycoprotein [Homo sapiens] |
| 419 | 1 | 167 | 16 | 516 | g2587024 | 3.00E−34 | HERV-E envelope glycoprotein [Homo sapiens] |
| 419 | 1 | 167 | 16 | 516 | g1049232 | 2.00E−31 | HERV-E envelope protein [Human endogenous retrovirus] |
| 420 | 2 | 59 | 227 | 403 | | | |
| 421 | 1 | 216 | 1 | 648 | g10504238 | 1.00E−101 | hepatocellular carcinoma-related putative tumor suppressor [Homo sapiens] |
| 421 | 1 | 216 | 1 | 648 | g7020759 | 7.00E−75 | unnamed protein product [Homo sapiens] |
| 421 | 1 | 216 | 1 | 648 | g3880143 | 1.00E−28 | contains similarity to Pfam domain: PF01585 (G-patch domain), Score = 67.0, E |
| | | | | | | | value = 1.3e−16, N = 1 [Caenorhabditis elegans] |
| 422 | 1 | 162 | 1 | 486 | g4982485 | 7.00E−55 | apoptosis related protein APR-3 [Homo sapiens] |
| 422 | 1 | 162 | 1 | 486 | g4689122 | 3.00E−49 | HSPC013 [Homo sapiens] |

TABLE 7

| | | | Parameter |
|---|---|---|---|
| Program | Description | Reference | Threshold |
| ABIFACTURA | A program that removes vector sequences and | Applied Biosystems, Foster City, CA. | |
| | masks ambiguous bases in nucleic acid sequences. | | |
| ABI/ | A Fast Data Finder useful in comparing and | Applied Biosystems, Foster City, CA; | Mismatch < |
| PARACEL | annotating amino acid or nucleic acid sequences. | Paracel Inc., Pasadena, CA. | 50% |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: |
| | sequence similarity search for amino acid and | 215: 403-410; Altschul, S. F. et al. (1997) | Probability |
| | nucleic acid sequences. BLAST includes five | Nucleic Acids Res. 25: 3389-3402. | value = 1.0E−8 |
| | functions: blastp, blastn, blastx, tblastn, and tblastx. | | or less Full |
| | | | Length |
| | | | sequences: |
| | | | Probability |
| | | | value = |
| | | | 1.0E−10 or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, | value = |
| | sequences of the same type. FASTA comprises as | W. R. (1990) Methods Enzymol. 183: 63-98; | 1.06E−6 |
| | least five functions: fasta, tfasta, fastx, tfastx, and | and Smith, T. F. and M. S. Waterman (1981) | Assembled |
| | ssearch. | Adv. Appl. Math. 2: 482-489. | ESTs: fasta |
| | | | Identity = 95% |
| | | | or greater and |
| | | | Match length = |
| | | | 200 bases or |
| | | | greater; fastx E |
| | | | value = 1.0E−8 |
| | | | or less Full |
| | | | Length |
| | | | sequences: |
| | | | fastx score = |
| | | | 100 or greater |
| BLIMPS | A BLocks IMProved Searcher that matches a | Henikoff, S. and J. G. Henikoff (1991) Nucleic | Probability |
| | sequence against those in BLOCKS, PRINTS, | Acids Res. 19: 6565-6572; Henikoff, J. G. and | value = 1.0E−3 |
| | DOMO, PRODOM, and PFAM databases to search | S. Henikoff (1996) Methods Enzymol. | or less |
| | for gene families, sequence homology, and structural | 266: 88-105; and Attwood, T. K. et al. (1997) J. | |
| | fingerprint regions. | Chem. Inf. Comput. Sci. 37: 417-424. | |
| HMMER | An algorithm for searching a query sequence against | Krogh, A. et al. (1994) J. Mol. Biol. | PFAM hits: |
| | hidden Markov model (HMM)-based databases of | 235: 1501-1531; Sonnhammer, E. L. L. et al. | Probability |
| | protein family consensus sequences, such as PFAM. | (1988) Nucleic Acids Res. 26: 320-322; | value = 1.0E−3 |
| | | Durbin, R. et al. (1998) Our World View, in a | or less |
| | | Nutshell, Cambridge Univ. Press, pp. 1-350. | Signal peptide |
| | | | hits: Score = 0 |
| | | | or greater |
| ProfileScan | An algorithm that searches for structural and sequence | Gribskov, M. et al. (1988) CABIOS 4: 61-66; | Normalized |
| | motifs in protein sequences that match sequence patterns | Gribskov, M. et al. (1989) Methods Enzymol. | quality score ≧ |
| | defined in Prosite. | 183: 146-159; Bairoch, A. et al. (1997) | GCG-specified |
| | | Nucleic Acids Res. 25: 217-221. | “HIGH” value |
| | | | for that |
| | | | particular |
| | | | Prosite motif. |
| | | | Generally, |
| | | | score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = 120 or |
| | CrossMatch, programs based on efficient implementation | Appl. Math. 2: 482-489; Smith, T.F. and M.S. | greater; |
| | of the Smith-Waterman algorithm, useful in searching | Waterman (1981) J. Mol. Biol. 147: 195-197; | Match length = |
| | sequence homology and assembling DNA sequences. | and Green, P., University of Washington, | 56 or greater |
| | | Seattle, WA. | |
| Consed | A graphical tool for viewing and editing Phrap assemblies. | Gordon, D. et al. (1998) Genome Res. 8: 195-202. | |
| SPScan | A weight matrix analysis program that scans protein | Nielson, H. et al. (1997) Protein Engineering | Score = 3.5 or |
| | sequences for the presence of secretory signal peptides. | 10: 1-6; Claverie, J.M. and S. Audic (1997) | greater |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. on Intelligent Systems for Mol. Biol., | |
| | and determine orientation. | Glasgow et al., eds., The Am. Assoc. for Artificial | |
| | | Intelligence Press, Menlo Park, CA, pp. 175-182. | |
| Motifs | A program that searches amino acid sequences for patterns | Bairoch, A. et al. (1997) Nucleic Acids | |
| | that matched those defined in Prosite. | Res. 25: 217-221; | |
| | | Wisconsin Package Program Manual, version 9, page | |
| | | M51-59, Genetics Computer Group, Madison, WI. | |

What is claimed is:

1. An isolated polynucleotide comprising a polynucleotide sequence selected from the group consisting of:
a) a polynucleotide sequence selected from the group consisting of SEQ ID NO:1-211,
b) a naturally occurring polynucleotide sequence having at least 90% sequence identity to a polynucleotide sequence selected from the group consisting of SEQ ID NO:1-211,
c) a polynucleotide sequence complementary to a),
d) a polynucleotide sequence complementary to b), and
e) an RNA equivalent of a) through d).

2. An isolated polynucleotide of claim 1, comprising a polynucleotide sequence selected from the group consisting of SEQ ID NO:1-211.

3. An isolated polynucleotide comprising at least 60 contiguous nucleotides of a polynucleotide of claim 1.

4. A composition for the detection of expression of diagnostic and therapeutic polynucleotides comprising at least one of the polynucleotides of claim 1 and a detectable label.

5. A method for detecting a target polynucleotide in a sample, said target polynucleotide having a sequence of a polynucleotide of claim 1, the method comprising:
a) amplifying said target polynucleotide or fragment thereof using polymerase chain reaction amplification, and
b) detecting presence or absence of said amplified target polynucleotide or fragment thereof, and, optionally, if preset the amount thereof.

6. A method for detecting a target polynucleotide in a sample, said target polynucleotide comprising a sequence of a polynucleotide of claim 1, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

7. A method of claim 5, wherein the probe comprises at least 30 contiguous nucleotides.

8. A method of claim 5, wherein the probe comprises at least 60 contiguous nucleotides.

9. A recombinant polynucleotide comprising a promoter sequence operably linked to a polynucleotide of claim 1.

10. A cell transformed with a recombinant polynucleotide of claim 9.

11. A transgenic organism comprising a recombinant polynucleotide of claim 9.

12. A method for producing a diagnostic and therapeutic polypeptide, the method comprising:
a) culturing a cell under conditions suitable for expression of the diagnostic and therapeutic polypeptide, wherein said cell is transformed with a recombinant polynucleotide of claim 9, and
b) recovering the diagnostic and therapeutic polypeptide so expressed.

13. A purified diagnostic and therapeutic polypeptide (DITHP) encoded by at least one of the polynucleotides of claim 2.

14. An isolated antibody which specifically binds to a diagnostic and therapeutic polypeptide of claim 13.

15. A method of identifying a test compound which specifically binds to the diagnostic and therapeutic polypeptide of claim 13, the method comprising the steps of:
a) providing a test compound;
b) combining the diagnostic and therapeutic polypeptide with the test compound for a sufficient time and under suitable conditions for binding; and
c) detecting binding of the diagnostic and therapeutic polypeptide to the test compound, thereby identifying the test compound which specifically binds the diagnostic and therapeutic polypeptide.

16. A microarray wherein at least one element of the microarray is a polynucleotide of claim 3.

17. A method for generating a transcript image of a sample which contains polynucleotides, the method comprising the steps of:
a) labeling the polynucleotides of the sample,
b) contacting the elements of the microarray of claim 16 with the labeled polynucleotides of the sample under conditions suitable for the formation of a hybridization complex, and
c) quantifying the expression of the polynucleotides in the sample.

18. A method for screening a compound for effectiveness in altering expression of a target polynucleotide, wherein said target polynucleotide comprises a polynucleotide sequence of claim 1, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

19. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 1 under conditions whereby a specific hybridization complex is formed between said probe and a target polynucleotide in the biological sample, said target polynucleotide comprising a polynucleotide sequence of a polynucleotide of claim 1 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.

20. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 1.

21. An array of claim 20, wherein said first oligonucleotide or polynucleotide sequence is completely complementary to at least 30 contiguous nucleotides of said target polynucleotide.

22. An array of claim 20, wherein said first oligonucleotide or polynucleotide sequence is completely complementary to at least 60 contiguous nucleotides of said target polynucleotide

23. An array of claim 20, which is a microarray.

24. An array of claim 20, further comprising said target polynucleotide hybridized to said first oligonucleotide or polynucleotide.

25. An array of claim 20, wherein a linker joins at least one of said nucleotide molecules to said solid substrate.

26. An array of claim 20, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.

27. An isolated polypeptide comprising an amino acid sequence selected from the group consisting of:
a) an amino acid sequence selected from the group consisting of SEQ ID NO:212-422,
b) a naturally occurring amino acid sequence having at least 90% sequence identity to an amino acid sequence selected from the group consisting of SEQ ID NO:212-422,
c) a biologically active fragment of an amino acid sequence selected from the group consisting of SEQ ID NO:212-422, and
d) an immunogenic fragment of an amino acid sequence selected from the group consisting of SEQ ID NO:212-422.
